(19) United States
(12) Patent Application Publication
(10) Pub. No.: US 2026/0028336 A1
(43) Pub. Date: Jan. 29, 2026

(54) CBL-B INHIBITORS AND METHODS OF USE THEREOF

(71) Applicant: Arcus Biosciences, Inc., Hayward, CA (US)

(72) Inventors: Joel Worley Beatty, San Mateo, CA (US); Balint Gal, Hayward, CA (US); Srikanth Kumar Gangam, Fremont, CA (US); Clayton Hardman, San Francisco, CA (US); Manmohan Reddy Leleti, Dublin, CA (US); Kenneth Victor Lawson, San Francisco, CA (US); Dongdong Liu, Fremont, CA (US); Artur Karenovich Mailyan, Hayward, CA (US); Masa Podunavac, Hayward, CA (US); Tzu-Yu Yu, Hayward, CA (US); Kai Yu, Hayward, CA (US); Xianglin Yin, Hayward, CA (US)

(73) Assignee: Arcus Biosciences, Inc., Hayward, CA (US)

(21) Appl. No.: 18/996,301

(22) PCT Filed: Jul. 18, 2023
(86) PCT No.: PCT/US2023/028034

§ 371 Date: Jan. 17, 2025

Related U.S. Application Data
(60) Provisional application No. 63/390,862, filed on Jul. 20, 2022.
(60) Provisional application No. 63/457,985, filed on Apr. 7, 2023.

(51) Int. Cl. C07D 471/04; A61K 31/4365; A61K 31/437; A61K 31/438; A61K 31/439; A61K 31/4545; A61K 31/496; A61K 31/4995; A61K 31/5025; A61K 31/519; A61K 31/5377; A61K 31/5386; C07D 487/04; C07D 495/04; C07D 519/00

(52) U.S. Cl. CPC C07D 471/04; A61K 31/4365; A61K 31/437; A61K 31/438; A61K 31/439; A61K 31/4545; A61K 31/496; A61K 31/4995; A61K 31/5025; A61K 31/519; A61K 31/5377; A61K 31/5386; C07D 487/04; C07D 495/04; C07D 519/00

(57) ABSTRACT

Disclosed herein are compounds that are Cbl-b inhibitors having a structure according to Formula (II), and compositions containing those compounds. Methods of preparing the compounds, and methods of using the compounds for the treatment of diseases, disorders, or conditions are also described.

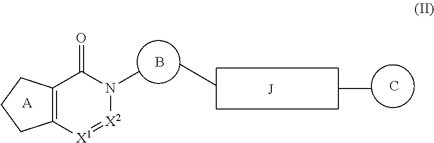

### CROSS-REFERENCE TO RELATED APPLICATIONS

This application claims the benefit of priority to U.S. Provisional Patent Application No. 63/390,862, filed on Jul. 20, 2022, and U.S. Provisional Patent Application No. 63/457,985, filed on Apr. 7, 2023, the entire content of each of which is incorporated by reference herein.

### BACKGROUND

The following discussion is provided to aid the reader in understanding the disclosure and is not admitted to describe or constitute prior art thereto.

Crucial to the proper regulation of immune responses is a balance between immune activating and inhibitory signals. One cellular mechanism that regulates diverse aspects of the immune system is ubiquitination. Ubiquitination involves covalent conjugation of monoubiquitin or polyubiquitin chains onto amino acid residues of target proteins. Protein ubiquitination can alter the activity and/or stability of a molecule, and in some instances also alter localization of the molecule into different cellular compartments. The ubiquitination process is catalyzed by sequential actions of ubiquitin-activating (E1), ubiquitin-conjugating (E2) and ubiquitin-ligating (E3) enzymes. The process of protein ubiquitination is counteracted by deubiquitinases (DUBs), a large family of proteases that cleaves ubiquitin chains. Mammalian cells express more than 600 E3 ligases and about 100 DUBs, which display substrate specificities and regulate specific cellular functions.

An increasing number of E3 ligases and DUBs have been identified as important regulators of immune responses. For example, small-molecule inhibitors that are antagonists of the IAP family of E3 ligases, including cIAP1, cIAP2, and X-linked IAP (XIAP), have been developed as small-molecule mimetics of the endogenous IAP inhibitor Smac. Small molecule inhibitors have also been developed against MDM2, an E3 ligase that promotes tumor growth and progression by mediating ubiquitin-dependent degradation of the tumor suppressor p53 and p53-independent functions. Casitas B-lineage lymphoma (Cbl) proteins, a family of E3 ubiquitin ligases, have been previously identified as potential targets; and so has VHL E3 complex, which mediates ubiquitin-dependent degradation of HIF1α and controls metabolic activities and effector function of T cells. Small molecule inhibitors for several DUBs have also been developed, and some of them have been shown to inhibit tumor growth in animal models.

While these potential targets provide exciting opportunities for the immunotherapy field, there remains a need in the art for effective inhibitors.

### SUMMARY

Casitas B-lineage lymphoma (Cbl) proteins are a family of E3 ubiquitin ligases. The mammalian Cbl family contains three homologs—c-Cbl, Cbl-b, and Cbl-3. Cbl-b and c-Cbl share some structural similarities but may have distinct physiological functions.

In one aspect, the present disclosure relates to compounds represented by Formula I:

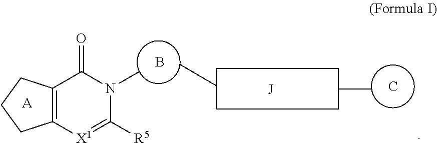

In one aspect, this disclosure is directed to a compound having a structure according to Formula II:

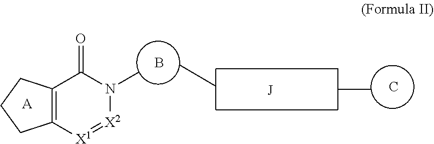

or a pharmaceutically acceptable salt thereof;
wherein:
ring A is selected from the group consisting of:

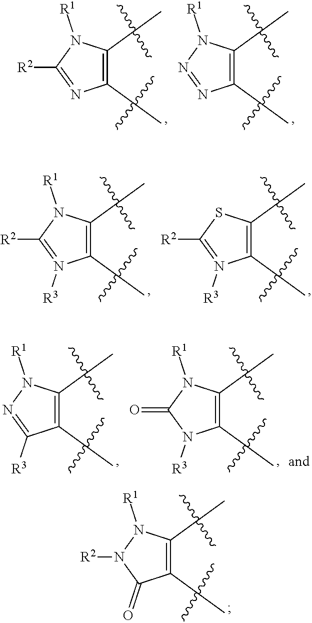

R¹, when present, is —H or C₁-C₃ alkyl;
R², when present, is H; —CN; —NR^{2a}R^{2b}, wherein R^{2a} and R^{2b} are independently selected from H and C₁-C₆ alkyl; —S(O)₂(C₁-C₃ alkyl); —S(O)(NR^{2c})(C₁-C₃ alkyl); —NR^{2c}—S(O)₂(C₁-C₃ alkyl); —S(O)₂—N(R^{2c})₂; 5- to 6-membered heteroaryl containing 1-3 ring heteroatoms independently selected from N, S, and O, and the 5- or 6-membered heteroaryl is substituted with 0-3 C₁-C₃ alkyl; 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl; unsubstituted C₁-C₃ alkyl; or C₁-C₃ alkyl substituted with (i) —OR^{2c}, (ii) —C(O)NR^{a}R^{b}, (iii) —NR^{a}R^{b}, (iv) a 4- to 8-membered heterocycloalkyl ring having 1-2 ring heteroatoms independently selected from N, S, and O, and the 4- to 8-membered heterocycloalkyl ring is substituted with 0-3 substituents independently selected from halo, —OH, C₁-C₃ alkyl, C₁-C₃ haloalkyl, and C₁-C₃ hydroxyalkyl, or (v) 5- to 6-membered heteroaryl containing 1-3 ring heteroatoms independently selected from N, S, and O; wherein R², is H or C₁-C₃ alkyl; and R^{a} and R^{b} are independently H, C₁-C₃ alkyl, phenyl, —(C₁-C₃ alkylene)-O—(C₁-C₃ alkyl), C₃-C₆ cycloalkyl, —(C₁-C₃ alkylene)-(C₃-C₆ cycloalkyl), —S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-O—(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2d}, when present, is independently halo, —OH, C₁-C₃ alkyl, C₁-C₃ hydroxyalkyl, or C₁-C₃ haloalkyl;
R³, when present, is H; —CN; C₁-C₃ alkyl; C₁-C₃ alkylene-NR^{c}R^{d}; C₁-C₃ haloalkyl; C₁-C₃ hydroxyalkyl; C₃-C₆ cycloalkyl; —S(O)₂(C₁-C₃ alkyl); —COR^{c}; —COOR^{c}; —CONR^{c}R^{d}; or 5-membered heteroaryl containing 1-3 ring heteroatoms independently selected from N, O, and S, and the 5-membered heteroaryl is substituted with 0-3 substituents independently selected from halo, OH, CN, C₁-C₃ alkyl, C₁-C₃ haloalkyl, C₁-C₃ hydroxyalkyl, —C(O)NR^{c}R^{d}, —NR^{c}R^{d}, —COR^{c}, —COOR^{c}, —S(O)₂(C₁-C₃ alkyl), —NR^{c}—S(O)₂(C₁-C₃ alkyl), and —S(O)₂—NR^{c}R^{d}; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl;
X¹ is CR⁴ or N;
X² is CR⁵ or N;
R⁴, when present, is H; —CN; halogen; C₁-C₃ alkyl; C₁-C₃ haloalkyl; C₁-C₃ hydroxyalkyl; C₂-C₃ alkenyl; C₃-C₄ cycloalkyl; —S(O)₂(C₁-C₃ alkyl); —C(O)OH; or 5- or 6-membered heteroaryl having 1 to 4 ring heteroatoms independently selected from N, S, and O, and the heteroaryl is substituted with 0-3 C₁-C₃ alkyl;
R⁵ is H or C₁-C₃ alkyl;
ring B is phenylene, or 5- to 6-membered heteroarylene having 1-3 ring heteroatoms independently selected from N, S, and O;
J is —(CR^{g}R^{h})ₙ—Y¹— or —Y¹—(CR^{g}R^{h})ₙ—; wherein n is 1 or 2; Y¹ is absent, CH₂, CHF, CF₂, O, S, S(O), or S(O)₂; and each R^{g} and each R^{h} is independently H, halo, —OH, C₁-C₃ alkyl, C₁-C₃ haloalkyl, C₁-C₃ alkoxy or C₃-C₄ cycloalkyl; or R^{g} and R^{h} together with the carbon atom to which they are attached form a 3- to 4-membered heterocycle ring having 1 ring heteroatom selected from N, O, and S, a C₃-C₄ cycloalkyl ring, or a C₆-C₈ spirocyclic ring, where each cycloalkyl ring or spirocyclic ring is substituted with 0-3 R^{j}; and each R^{j} is independently selected from —CN, —OH, halogen, C₁-C₃ alkyl, C₁-C₃ alkoxy, and C₁-C₃ haloalkoxy; and
ring C is 5- to 6-membered heteroaryl containing 1-3 ring heteroatoms independently selected from N, S, and O, and the 5- to 6-membered heteroaryl is substituted with 0-3 substituents independently selected from C₁-C₃ alkyl, C₁-C₃ haloalkyl, and C₃-C₄ cycloalkyl.

In another aspect, this disclosure is directed to methods of inhibiting Cbl-b in a subject comprising administering to the subject an effective amount of a compound described herein.

In another aspect, this disclosure is directed to methods of increasing immune cell activity in a subject comprising administering to the subject an effective amount of a compound described herein.

In yet another aspect, this disclosure provides methods for treating a disease, disorder, or condition mediated at least in part by Cbl-b in a subject, comprising administering to the subject a therapeutically effective amount of a compound described herein. Diseases, disorders, and conditions mediated by Cbl-b include cancer and cancer-related disorders.

Certain aspects of the present disclosure further comprise the administration of one or more additional therapeutic agents as set forth herein below.

### DETAILED DESCRIPTION OF THE DISCLOSURE

Before the present disclosure is further described, it is to be understood that the disclosure is not limited to the particular embodiments set forth herein, and it is also to be understood that the terminology used herein is for the purpose of describing particular embodiments only, and is not intended to be limiting.

### Definitions

Unless otherwise defined, all terms of art, notations and other scientific terms or terminology used herein are intended to have the meanings commonly understood by those of skill in the art to which this disclosure pertains.

The term “about” as used herein has its original meaning of approximately and is to provide literal support for the exact number that it precedes, as well as a number that is near to or approximately the number that the term precedes. In general, the term “about” refers to the usual error range for the respective value readily known to the skilled person in this technical field. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The term “alkyl”, by itself or as part of another substituent, means, unless otherwise stated, a saturated monovalent hydrocarbon radical, having, in some embodiments, one to eight (e.g., C₁-C₈ alkyl), or one to six (e.g., C₁-C₆ alkyl), or one to three (e.g., C₁-C₃ alkyl) carbon atoms, respectively. The term “alkyl” encompasses straight and branched-chain hydrocarbon groups. Examples of alkyl groups include, but are not limited to, methyl, ethyl, n-propyl, isopropyl, n-butyl, t-butyl, isobutyl, sec-butyl, isopentyl, tert-pentyl, n-pentyl, isohexyl, n-hexyl, n-heptyl, 4-isopropylheptane, n-octyl, and the like. In some embodiments, the alkyl groups are C₁-C₄ alkyl groups (e.g., methyl, ethyl, isopropyl, or t-butyl). In some embodiments, the alkyl groups are C₁-C₃ alkyl groups (e.g., methyl, ethyl, n-propyl, or iso-propyl).

The term “alkenyl”, as used herein, refers to a straight or branched monovalent hydrocarbon radical having, in some embodiments, two to eight carbon atoms (e.g., C₂-C₈ alkenyl), or two to six carbon atoms (e.g., C₂-C₆ alkenyl), or two to three carbon atoms (e.g., C₂-C₃ alkenyl), and having at least one carbon-carbon double bond. Examples of alkenyl groups include, but are not limited to, ethenyl, propenyl, isobutenyl, butadienyl and the like.

The term “alkylene” refers to a straight or branched, saturated, hydrocarbon radical having, in some embodiments, one to six (e.g., C₁-C₆ alkylene), one to four (e.g., C₁-C₄ alkylene), or one to two (e.g., C₁-C₂ alkylene) carbon atoms, and linking at least two other groups, i.e., a divalent hydrocarbon radical. When two moieties are linked to the alkylene they can be linked to the same carbon atom (i.e., geminal), or different carbon atoms of the alkylene group. For instance, a straight chain alkylene can be the bivalent radical of —(CH₂)ₙ—, where n is 1, 2, 3, 4, 5 or 6 (i.e., a C₁-C₆ alkylene). Representative alkylene groups include, but are not limited to, methylene, ethylene, propylene, isopropylene, butylene, isobutylene, secbutylene, pentylene, hexylene and the like. In some embodiments, the alkylene groups are C₁-C₂ alkylene groups (e.g., methylene, or ethylene).

As used herein, the term “alkoxy” refers to an alkyl group, as defined herein, that is attached to the remainder of the molecule via an oxygen atom (e.g., —O—C₁-C₁₂ alkyl, —O—C₁-C₈ alkyl, —O—C₁-C₆ alkyl, or —O—C₁-C₃ alkyl). Non-limiting examples of alkoxy groups include methoxy, ethoxy, n-propoxy, iso-propoxy, n-butoxy, sec-butoxy, n-pentoxy, n-hexoxy, and the like. In some embodiments, the alkoxy groups are C₁-C₃ alkoxy groups (e.g., methoxy, ethoxy, n-propoxy, or iso-propoxy).

The term “cycloalkyl” refers to a monocyclic, bicyclic or polycyclic hydrocarbon ring system having, in some embodiments, 3 to 14 carbon atoms (e.g., C₃-C₁₄ cycloalkyl), or 3 to 10 carbon atoms (e.g., C₃-C₁₀ cycloalkyl), or 3 to 8 carbon atoms (e.g., C₃-C₈ cycloalkyl), or 3 to 6 carbon atoms (e.g., C₃-C₆ cycloalkyl) or 5 to 6 carbon atoms (e.g., C₅-C₆ cycloalkyl). Cycloalkyl groups can be saturated or characterized by one or more points of unsaturation (i.e., carbon-carbon double and/or triple bonds), provided that the points of unsaturation do not result in an aromatic system. Examples of monocyclic cycloalkyl groups include, but are not limited to, cyclopropyl, cyclobutyl, cyclopentyl, cyclohexyl, cyclohexeneyl, cyclohexynyl, cycloheptyl, cyclohepteneyl, cycloheptadieneyl, cyclooctyl, cycloocteneyl, cyclooctadieneyl and the like. The rings of bicyclic and polycyclic cycloalkyl groups can be fused, bridged, or spirocyclic. Non-limiting examples of bicyclic, spirocyclic and polycyclic hydrocarbon groups include bicyclo[2.2.1]heptane, bicyclo[2.2.2]octane, adamantyl, indanyl, spiro[5.5]undecane, spiro[2.2]pentane, spiro[2.2]pentadiene, spiro[2.3]hexane, spiro[2.5]octane, spiro[2.2]pentadiene, and the like. In some embodiments, the cycloalkyl groups of the present disclosure are monocyclic C₃-C₆cycloalkyl moieties (e.g., cyclopropyl, cyclobutyl, cyclopentyl, or cyclohexyl), or C₆-C₈ spirocyclic moieties (e.g., spiro[2.3]hexane).

The term “heterocycloalkyl” refers to a non-aromatic monocyclic, bicyclic or polycyclic cycloalkyl ring having, in some embodiments, 3 to 14 members (e.g., 3- to 14-membered heterocycle), or 3 to 10 members (e.g., 3- to 10-membered heterocycle), or 3 to 8 members (e.g., 3- to 8-membered heterocycle), or 3 to 6 members (e.g., 3- to 6-membered heterocycle), or 5 to 6 members (e.g., 5- to 6-membered heterocycle), and having from one to five, one to four, one to three, one to two or one heteroatom or heteroatom groups independently selected from nitrogen (N), oxygen (O), sulfur (S), sulfoxide (S(O)), and sulfone (S(O)₂). Heterocycloalkyl groups are saturated or characterized by one or more points of unsaturation (e.g., one or more carbon-carbon double bonds, carbon-carbon triple bonds, carbon-nitrogen double bonds, and/or nitrogen-nitrogen double bonds), provided that the points of unsaturation do not result in an aromatic system. The rings of bicyclic and polycyclic heterocycloalkyl groups can be fused, bridged, or spirocyclic. Non-limiting examples of heterocycloalkyl groups include aziridine, oxirane, thiirane, pyrrolidine, imidazolidine, pyrazolidine, dioxolane, phthalimide, piperidine, 1,4-dioxane, morpholine, thiomorpholine, thiomorpholine-S-oxide, thiomorpholine-S,S-oxide, piperazine, 3,4,5,6-tetrahydropyridazine, tetrahydropyran, pyran, decahydroisoquinoline, 3-pyrroline, thiopyran, tetrahydrofuran, tetrahydrothiophene, tetrahydro-1,1-dioxido-2H-thiopyran, quinuclidine, 2-azabicyclo[4.1.0]heptane, 2-oxa-5-azabicyclo[2.2.1]heptane, 2-azabicyclo[2.2.1]heptane, 2,5-diazabicyclo[2.2.1]heptane, 6-oxa-3-azabicyclo[3.1.1]heptane, 3-oxa-6-azabicyclo[3.1.1]heptane, 2,5-diazabicyclo[2.2.1]heptane, 2,6-diazaspiro[3.3]heptane, 2-azaspiro[3.3]heptane, 1-oxaspiro[3.3]heptane, 5-azaspiro[2.4]heptane, 6-azaspiro[3.4]octane, 6-azaspiro[2.5]octane, 4-oxa-7-azaspiro[2.5]octane, 3-oxa-8-azabicyclo[3.2.1]octane, and the like. A heterocycloalkyl group can be attached to the remainder of the molecule through a ring carbon atom, or a ring heteroatom, when chemically permissible. In some embodiments, the heterocycloalkyl groups of the present disclosure are monocyclic 4- to 8-membered heterocycloalkyl moieties having one or two heteroatom or heteroatom groups independently selected from N, and O (e.g., azetidine, piperidine, piperazine, morpholine, pyrrolidine, imidazolidine, pyrazolidine, tetrahydrofuran, tetrahydropyran, tetrahydro-1,1-dioxido-2H-thiopyran, 2-azabicyclo[4.1.0]heptane, 2-oxa-5-azabicyclo[2.2.1]heptane, 2,5-diazabicyclo[2.2.1]heptane, 6-oxa-3-azabicyclo[3.1.1]heptane, 3-oxa-6-azabicyclo[3.1.1]heptane, 2,5-diazabicyclo[2.2.1]heptane, 5-azaspiro[2.4]heptane, 6-azaspiro[2.5]octane, 4-oxa-7-azaspiro[2.5]octane, and the like).

The term “aryl” refers to an aromatic ring system containing one ring, or two or three rings fused together, and having, in some embodiments, six to fourteen (i.e., C₆-C₁₄ aryl), or six to ten (i.e., C₆-C₁₀ aryl), or six (i.e., C₆ aryl) carbon atoms. Non-limiting examples of aryl groups include phenyl, naphthyl and anthracenyl. In some embodiments, aryl groups are phenyl.

The term “phenylene” refers to a divalent phenyl group.

The term “heteroaryl” refers to monocyclic or fused bicyclic aromatic groups (or rings) having, in some embodiments, from 5 to 14 (i.e., 5- to 14-membered heteroaryl), or from 5 to 10 (i.e., 5- to 10-membered heteroaryl), or from 5 to 6 (i.e., 5- to 6-membered heteroaryl) members (i.e., ring vertices), and containing from one to five, one to four, one to three, one to two or one heteroatom independently selected from nitrogen (N), oxygen (O), and sulfur (S). A heteroaryl group can be attached to the remainder of the molecule through a carbon atom or a heteroatom of the heteroaryl group, when chemically permissible. Non-limiting examples of heteroaryl groups include pyridyl, pyridazinyl, pyrazinyl, pyrimindinyl, triazinyl, purinyl, thienopyridinyl, thienopyrimidinyl, pyrazolopyrimidinyl, imidazopyridines, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. In some embodiments, the heteroaryl groups of the present disclosure are monocyclic 5- to 6-membered heteroaryl moieties having 1-3 heteroatoms independently selected from N, O, and S (e.g., pyridinyl, pyrimidinyl, pyridazinyl, triazolyl, imidazolyl, pyrazolyl, oxazolyl, oxadiazolyl, or thiazolyl).

The term “heteroarylene” refers to a divalent heteroaryl group as defined herein. Exemplary heteroarylene groups include, but are not limited to pyrazolylene, oxazolylene, oxadiazolylene, imidazolylene, triazolylene, thiazolylene, pyrrolylene, furanylene, thiophenylene, pyridylene, pyrimidinylene, pyridazinylene, and the like. In some embodiments, the heteroarylene is a 6-membered heteroarylene having 1-3 ring heteroatoms selected from N, O, and S. In some embodiments, the heteroarylene is a 6-membered heteroarylene having 1-2 ring N atoms (e.g., pyridylene, pyrimidylene, pyridazinylene). In some embodiments, the heteroarylene is pyridylene.

[symbol]

As used herein, a wavy line, “ [symbol] ”, that intersects a single, double or triple bond in any chemical structure depicted herein, represents that the point of attachment of the single, double, or triple bond to the remainder of the molecule is through either one of the atoms that make up the single, double or triple bond. Additionally, a bond extending from a substituent to the center of a ring (e.g., a phenyl ring) is meant to indicate attachment of that substituent to the ring at any of the available ring vertices, i.e., such that attachment of the substituent to the ring results in a chemically stable arrangement.

The term “halogen,” by itself or as part of another substituent, means, unless otherwise stated, a fluorine, chlorine, bromine, or iodine atom. Additionally, terms such as “haloalkyl,”, and “haloalkoxy” refer to alkyl groups and alkoxy groups, respectively, as defined herein, that are substituted with one or more halogen(s) (e.g., 1-3 halogen(s)). For example, the term “C₁-C₄ haloalkyl” is meant to include trifluoromethyl, difluoromethyl, 2,2,2-trifluoroethyl, 4-chlorobutyl, 3-bromopropyl, and the like. As another example, the term “C₁-C₃ haloalkoxy” is meant to include trifluoromethoxy, difluoromethoxy, 2,2,2-trifluoroethoxy, 2,2-difluoroethoxy, and the like.

The term “hydroxyalkyl” refers to an alkyl group, as defined herein, that is substituted with one or more hydroxyl groups (e.g., 1-3 hydroxyl groups). Exemplary hydroxyalkyl groups include methanol, ethanol, 1,2-propanediol, 1,2-hexanediol, glycerol, and the like.

The compounds of the present disclosure can be present in their neutral form, or as a pharmaceutically acceptable salt, isomer, polymorph or solvate thereof, and may be present in a crystalline form, amorphous form or mixtures thereof.

As referred to herein, “pharmaceutically acceptable salt” is meant to include salts of the compounds according to this disclosure that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal ofPharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

This disclosure also contemplates isomers of the compounds described herein (e.g., stereoisomers). For example, certain compounds of the present disclosure possess asymmetric carbon atoms (chiral centers); the racemates, diastereomers, and enantiomers of which are all intended to be encompassed within the scope of the present disclosure. Stereoisomeric forms may be defined, in terms of absolute stereochemistry, as (R) or (S), and/or depicted uses dashes and/or wedges. When a stereochemical depiction (e.g., using dashes, [symbol] , and/or wedges, [symbol] ) is shown in a chemical structure, or a stereochemical assignment (e.g., using (R) and (S) notation) is made in a chemical name, it is meant to indicate that the depicted isomer is present and substantially free of one or more other isomer(s) (e.g., enantiomers and diastereomers, when present). “Substantially free of” other isomer(s) indicates at least an 70/30 ratio of the indicated isomer to the other isomer(s), more preferably 80/20, 90/10, or 95/5 or more. In some embodiments, the indicated isomer will be present in an amount of at least 99%. A chemical bond to an asymmetric carbon that is depicted as a solid line ( [symbol] ) indicates that all possible stereoisomers (e.g., enantiomers, diastereomers, racemic mixtures, etc.) at that carbon atom are included. In such instances, the compound may be present as a racemic mixture, scalemic mixture, or a mixture of diastereomers.

The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere herein. For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics which can contribute to enhanced safety, tolerability or efficacy during treatment. All isotopic variations of the compounds of the present disclosure, whether radioactive or not, are intended to be encompassed within the scope of the present disclosure. In some embodiments, the compounds according to this disclosure are characterized by one or more deuterium atoms.

The terms “patient” or “subject” are used interchangeably to refer to a human or a non-human animal (e.g., a mammal).

The terms “treat”, “treating”, treatment” and the like refer to a course of action that eliminates, reduces, suppresses, mitigates, ameliorates, or prevents the worsening of, either temporarily or permanently, a disease, disorder or condition to which the term applies, or at least one of the symptoms associated therewith. Treatment includes alleviation of symptoms, diminishment of extent of disease, inhibiting (e.g., arresting the development or further development of the disease, disorder or condition or clinical symptoms association therewith) an active disease, delaying or slowing of disease progression, improving the quality of life, and/or prolonging survival of a subject as compared to expected survival if not receiving treatment or as compared to a published standard of care therapy for a particular disease.

The term “in need of treatment” as used herein refers to a judgment made by a physician or similar professional that a subject requires or will benefit from treatment. This judgment is made based on a variety of factors that are in the realm of the physician's expertise, which may include a positive diagnosis of a disease, disorder or condition.

The terms “prevent”, “preventing”, “prevention”, “prophylaxis” and the like refer to a course of action initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state. Prevention also refers to a course of action initiated in a subject after the subject has been treated for a disease, disorder, condition or a symptom associated therewith in order to prevent relapse of that disease, disorder, condition or symptom.

The term “in need of prevention” as used herein refers to a judgment made by a physician or other caregiver that a subject requires or will benefit from preventative care. This judgment is made based on a variety of factors that are in the realm of a physician's or caregiver's expertise.

“Substantially pure” indicates that a component (e.g., a compound according to this disclosure) makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the component of interest will make up greater than about 90%, or greater than about 95% of the total content of the composition.

Compounds that are selective may be particularly useful in the treatment of certain disorders or may offer a reduced likelihood of undesired side effects.

Compounds provided herein may have advantageous pharmacokinetic profiles including, for example, metabolic liabilities, hepatocyte stability, clearance, and inhibition against CYP.

### Compounds of the Disclosure

The present disclosure relates to compounds that inhibit the activity of Cbl-b.

In one aspect, this disclosure is directed to a compound having a structure according to Formula II:

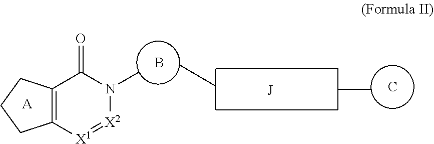

or a pharmaceutically acceptable salt thereof;
wherein:
ring A is selected from the group consisting of:

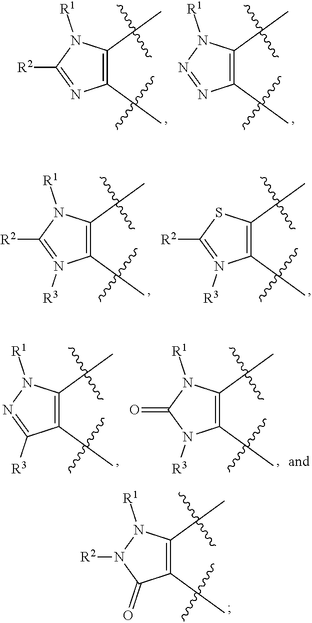

R¹, when present, is —H or C₁-C₃ alkyl;
R², when present, is H; —CN; —NR^{2a}R^{2b}, wherein R^{2a} and R^{2b} are independently selected from H and C₁-C₆ alkyl; —S(O)₂(C₁-C₃ alkyl); —S(O)(NR^{2c})(C₁-C₃ alkyl); —NR^{2c}—S(O)₂(C₁-C₃ alkyl); —S(O)₂—N(R^{2c})₂; 5- to 6-membered heteroaryl containing 1-3 ring heteroatoms independently selected from N, S, and O, and the 5- or 6-membered heteroaryl is substituted with 0-3 C₁-C₃ alkyl; 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl; unsubstituted C₁-C₃ alkyl; or C₁-C₃ alkyl substituted with (i) —OR^{2c}, (ii) —C(O)NR^{a}R^{b}, (iii) —NR^{a}R^{b}, (iv) a 4- to 8-membered heterocycloalkyl ring having 1-2 ring heteroatoms independently selected from N, S, and O, and the 4- to 8-membered heterocycloalkyl ring is substituted with 0-3 substituents independently selected from halo, —OH, C₁-C₃ alkyl, C₁-C₃ haloalkyl, and C₁-C₃ hydroxyalkyl, or (v) 5- to 6-membered heteroaryl containing 1-3 ring heteroatoms independently selected from N, S, and O; wherein R², is H or C₁-C₃ alkyl; and R^{a} and R are independently H, C₁-C₃ alkyl, phenyl, —(C₁-C₃ alkylene)-O—(C₁-C₃ alkyl), C₃-C₆ cycloalkyl, —(C₁-C₃ alkylene)-(C₃-C₆ cycloalkyl), —S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-O—(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2d}, when present, is independently halo, —OH, C₁-C₃ alkyl, C₁-C₃ hydroxyalkyl, or C₁-C₃ haloalkyl;
R³, when present, is H; —CN; C₁-C₃ alkyl; C₁-C₃ alkylene-NR^{c}R^{d}; C₁-C₃ haloalkyl; C₁-C₃ hydroxyalkyl; C₃-C₆ cycloalkyl; —S(O)₂(C₁-C₃ alkyl); —COR^{c}; —COOR^{c}; —CONR^{c}R^{d}; or 5-membered heteroaryl containing 1-3 ring heteroatoms independently selected from N, O, and S, and the 5-membered heteroaryl is substituted with 0-3 substituents independently selected from halo, OH, CN, C₁-C₃ alkyl, C₁-C₃ haloalkyl, C₁-C₃ hydroxyalkyl, —C(O)NR^{c}R^{d}, —NR^{c}R^{d}, —COR^{c}, —COOR^{c}, —S(O)₂(C₁-C₃ alkyl), —NR^{c}—S(O)₂(C₁-C₃ alkyl), and —S(O)₂—NR^{c}R^{d}; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl;
X¹ is CR⁴ or N;
X² is CR⁵ or N;
R⁴, when present, is H; —CN; halogen; C₁-C₃ alkyl; C₁-C₃ haloalkyl; C₁-C₃ hydroxyalkyl; C₂-C₃ alkenyl; C₃-C₄ cycloalkyl; —S(O)₂(C₁-C₃ alkyl); —C(O)OH; or 5- or 6-membered heteroaryl having 1 to 4 ring heteroatoms independently selected from N, S, and O, and the heteroaryl is substituted with 0-3 C₁-C₃ alkyl;
R⁵ is H or C₁-C₃ alkyl;
ring B is phenylene, or 5- to 6-membered heteroarylene having 1-3 ring heteroatoms independently selected from N, S, and O;
J is —(CR^{g}R^{h})ₙ—Y¹— or —Y¹—(CR^{g}R^{h})ₙ—; wherein n is 1 or 2; Y¹ is absent, CH₂, CHF, CF2, O, S, S(O), or S(O)₂; and each R^{g} and each R^{h} is independently H, halo, —OH, C₁-C₃ alkyl, C₁-C₃ haloalkyl, C₁-C₃ alkoxy or C₃-C₄ cycloalkyl; or R^{g} and R^{h} together with the carbon atom to which they are attached form a 3- to 4-membered heterocycle ring having 1 ring heteroatom selected from N, O, and S, a C₃-C₄ cycloalkyl ring, or a C₆-C₈ spirocyclic ring, where each cycloalkyl ring or spirocyclic ring is substituted with 0-3 R^{j}; and each R^{j} is independently selected from —CN, —OH, halogen, C₁-C₃ alkyl, C₁-C₃ alkoxy, and C₁-C₃ haloalkoxy; and
ring C is 5- to 6-membered heteroaryl containing 1-3 ring heteroatoms independently selected from N, S, and O, and the 5- to 6-membered heteroaryl is substituted with 0-3 substituents independently selected from C₁-C₃ alkyl, C₁-C₃ haloalkyl, and C₃-C₄ cycloalkyl.

In some embodiments, this disclosure is directed to a compound having a structure according to Formula II:

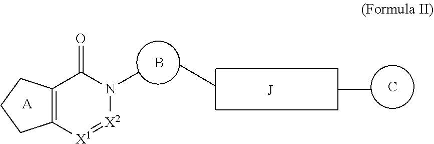

or a pharmaceutically acceptable salt thereof;
wherein:
ring A is selected from the group consisting of:

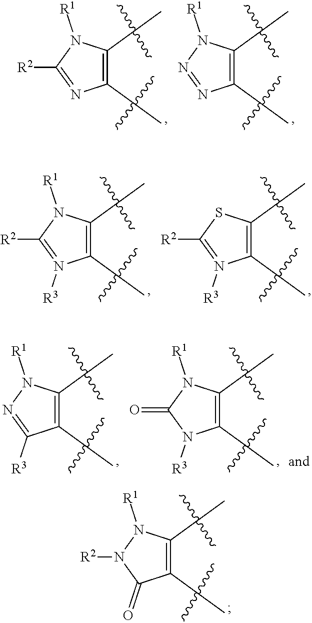

R¹, when present, is —H or C₁-C₃ alkyl;
R², when present, is H; —CN; —NR^{2a}R^{2b} wherein R^{2a} and R^{2b} are independently selected from H and C₁-C₆ alkyl; —S(O)₂(C₁-C₃ alkyl); —S(O)(NR^{2c})(C₁-C₃ alkyl); —NR^{2c}—S(O)₂(C₁-C₃ alkyl); —S(O)₂—N(R^{2c})₂; 5- to 6-membered heteroaryl containing 1-3 ring heteroatoms independently selected from N, S, and O, and the 5- or 6-membered heteroaryl is substituted with 0-3 C₁-C₃ alkyl; 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl; unsubstituted C₁-C₃ alkyl; or C₁-C₃ alkyl substituted with (i) —OR^{2c}, (ii) —C(O)NR^{a}R^{b}, (iii) —NR^{a}R^{b}, (iv) a 4- to 8-membered heterocycloalkyl ring having 1-2 ring heteroatoms independently selected from N, S, and O, and the 4- to 8-membered heterocycloalkyl ring is substituted with 0-3 substituents independently selected from halo, —OH, C₁-C₃ alkyl, C₁-C₃ haloalkyl, and C₁-C₃ hydroxyalkyl, or (v) 5- to 6-membered heteroaryl containing 1-3 ring heteroatoms independently selected from N, S, and O; wherein R^{2c} is H or C₁-C₃ alkyl; and R^{a} and R^{b} are independently H, C₁-C₃ alkyl, phenyl, —(C₁-C₃ alkylene)-O—(C₁-C₃ alkyl), C₃-C₆ cycloalkyl, —(C₁-C₃ alkylene)-(C₃-C₆ cycloalkyl), S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-O—(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2a}, when present, is independently halo, —OH, C₁-C₃ alkyl, C₁-C₃ hydroxyalkyl, or C₁-C₃ haloalkyl;
R³, when present, is H; —CN; C₁-C₃ alkyl; C₁-C₃ alkylene-NR^{c}R^{d}; C₁-C₃ haloalkyl; C₁-C₃ hydroxyalkyl; C₃-C₆ cycloalkyl; —S(O)₂(C₁-C₃ alkyl); —COR^{c}; —COOR^{c}; —CONR^{c}R^{d}; or 5-membered heteroaryl containing 1-3 ring heteroatoms independently selected from N, O, and S, and the 5-membered heteroaryl is substituted with 0-3 substituents independently selected from halo, OH, CN, C₁-C₃ alkyl, C₁-C₃ haloalkyl, C₁-C₃ hydroxyalkyl, —C(O)NR^{c}R^{d}, —NR^{c}R^{d}, —COR^{c}, —COOR^{c}, —S(O)₂(C₁-C₃ alkyl), —NR^{c}—S(O)₂(C₁-C₃ alkyl), and —S(O)₂—NR^{c}R^{d}; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl;
X¹ is CR⁴ or N;
X² is CR⁵ or N;
R⁴, when present, is H; —CN; halogen; C₁-C₃ alkyl; C₁-C₃ haloalkyl; C₁-C₃ hydroxyalkyl; C₂-C₃ alkenyl; C₃-C₄ cycloalkyl; —S(O)₂(C₁-C₃ alkyl); —C(O)OH; or 5- or 6-membered heteroaryl having 1 to 4 ring heteroatoms independently selected from N, S, and O, and the heteroaryl is substituted with 0-3 C₁-C₃ alkyl;
R⁵ is H or C₁-C₃ alkyl;
ring B is phenylene, or 5- to 6-membered heteroarylene having 1-3 ring heteroatoms independently selected from N, S, and O;
J is —(CR^{g}R^{h})ₙ—Y¹— or —Y¹—(CR^{g}R^{h})ₙ—; wherein n is 1 or 2; Y¹ is absent, CH₂, CHF, CF2, O, S, S(O), or S(O)₂; and each R^{g} and each R^{h} is independently H, C₁-C₃ alkyl, or C₃-C₄ cycloalkyl; or R^{g} and R^{h} together with the carbon atom to which they are attached form a 3- to 4-membered heterocycle ring having 1 ring heteroatom selected from N, O, and S, C₃-C₄ cycloalkyl ring, or a C₆-C₈ spirocyclic ring, where each cycloalkyl ring or spirocyclic ring is substituted with 0-3 R^{j}; and each R^{j} is independently selected from —CN, —OH, halogen, C₁-C₃ alkyl, C₁-C₃ alkoxy, and C₁-C₃ haloalkoxy; and
ring C is 5- to 6-membered heteroaryl containing 1-3 ring heteroatoms independently selected from N, S, and O, and the 5- to 6-membered heteroaryl is substituted with 0-3 substituents independently selected from C₁-C₃ alkyl, C₁-C₃ haloalkyl, and C₃-C₄ cycloalkyl.

In some embodiments, R², when present, is H; 5- to 6-membered heteroaryl containing 1-3 ring heteroatoms independently selected from N, S, and O, and the 5- or 6-membered heteroaryl is optionally substituted with 1-3 substituents independently selected from C₁-C₃ alkyl; 5- or 6-membered heterocycloalkyl ring optionally having 1-2 ring heteroatoms independently selected from N, S, and O, and the 5- or 6-membered heterocycloalkyl ring is optionally substituted with 1-3 substituents independently selected from C₁-C₃ alkyl; or C₁-C₃ alkyl optionally substituted with —OH, —NR^{a}R^{b}, or a 4- or 8-membered heterocycloalkyl ring optionally having 1-2 ring heteroatoms independently selected from N, S, and O, wherein the 4- or 8-membered heterocycloalkyl ring is optionally substituted with 1-3 substituents independently selected from —OH, C₁-C₃ alkyl, halo, C₁-C₃ haloalkyl, and C₁-C₃ hydroxyalkyl; and R^{a} and R^{b} are independently H, C₁-C₃ alkyl, phenyl, —(C₁-C₃ alkylene)-O—(C₁-C₃ alkyl), C₃-C₆ cycloalkyl, —(C₁-C₃ alkylene)-(C₃-C₆ cycloalkyl), S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-O—(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2d}, when present, is independently halo, —OH, C₁-C₃ alkyl, C₁-C₃ hydroxyalkyl, or C₁-C₃ haloalkyl.

In some embodiments, R² is H. In some embodiments, R² is —CN. In some embodiments, R² is —NR^{2a}R^{2b}, wherein R^{2a} and R^{2b} are independently selected from H and C₁-C₆ alkyl. In some embodiments, R² is —S(O)₂—C₁-C₃ alkyl. In some embodiments, R² is —S(O)(NR^{2c})—C₁-C₃ alkyl. In some embodiments, R² is —NR^{2c}S(O)₂—C₁-C₃ alkyl. In some embodiments, R² is —S(O)₂—N(R^{2c})₂. In some embodiments, R² is 5- to 6-membered heteroaryl containing 1-3 ring heteroatoms independently selected from N, S, and O, and the 5- or 6-membered heteroaryl is substituted with 0-3 C₁-C₃ alkyl. In some embodiments, R² is 5-membered heteroaryl containing 1-3 ring heteroatoms independently selected from N, S, and O, and the 5-membered heteroaryl is substituted with 0-3 C₁-C₃ alkyl. In some embodiments, R² is 5- or 6-membered heterocycloalkyl ring optionally having 1-2 ring heteroatoms independently selected from N, S, and O, and the 5- or 6-membered heterocycloalkyl ring is substituted with 0-3 C₁-C₃ alkyl. In some embodiments, R² is 6-membered heterocycloalkyl ring having 1-2 ring heteroatoms independently selected from N, S, and O, and the 6-membered heterocycloalkyl ring is substituted with 0-3 C₁-C₃ alkyl. In some embodiments, R² is a C₁-C₃ alkyl.

In some embodiments, R² is C₁-C₃ alkyl substituted with (i) —OR^{2c}, (ii) —C(O)NR^{a}R^{b}, (iii) —NR^{a}R^{b}, (iv) a 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl, or (v) 5- to 6-membered heteroaryl containing 1-3 ring heteroatoms independently selected from N, S, and O; wherein R^{2e} is H or C₁-C₃ alkyl; and R^{a} and R^{b} are independently H or C₁-C₃ alkyl. In some embodiments, R² is C₁-C₃ alkyl substituted with —OR^{2c}, wherein R^{2c} is H or C₁-C₃ alkyl. In some embodiments, R² is C₁-C₃ alkyl substituted with —OH. In some embodiments, R² is C₁-C₃ alkyl substituted with —C(O)NR^{a}R^{b}, wherein R^{a} and R^{b} are independently H or C₁-C₃ alkyl. In some embodiments, R² is C₁-C₃ alkyl substituted with —NR^{a}R^{b}, wherein R^{a} and R^{b} are independently H or C₁-C₃ alkyl. In some embodiments, R² is C₁-C₃ alkyl substituted with a 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl. In some embodiments, R² is C₁-C₃ alkyl substituted with a 5- to 6-membered heteroaryl containing 1-3 ring heteroatoms independently selected from N, S, and O.

In some embodiments, R² is C₁-C₃ alkyl substituted with (i) —OR^{2c}, (ii) —C(O)NR^{a}R^{b}, (iii) —NR^{a}R^{b}, (iv) a 4- or 8-membered heterocycloalkyl ring having 1-2 ring heteroatoms independently selected from N, S, and O, and the 4- or 8-membered heterocycloalkyl ring is substituted with 0-3 substituents independently selected from halo, —OH, C₁-C₃ alkyl, C₁-C₃ haloalkyl, and C₁-C₃ hydroxyalkyl or (v) 5- to 6-membered heteroaryl containing 1-3 ring heteroatoms independently selected from N, S, and O; wherein R^{2c} is H or C₁-C₃ alkyl; and R^{a} and R^{b} are independently H, C₁-C₃ alkyl, phenyl, —(C₁-C₃ alkylene)-O—(C₁-C₃ alkyl), C₃-C₆ cycloalkyl, —(C₁-C₃ alkylene)-(C₃—C₆ cycloalkyl), S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-O—(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2d}, when present, is independently halo, —OH, C₁-C₃ alkyl, C₁-C₃ hydroxyalkyl, or C₁-C₃ haloalkyl. In some embodiments, R² is C₁-C₃ alkyl substituted with —OR^{2c}, wherein R^{2c} is H or C₁-C₃ alkyl. In some embodiments, R² is C₁-C₃ alkyl substituted with —OH. In some embodiments, R² is C₁-C₃ alkyl substituted with —C(O)NR^{a}R^{b}, wherein R^{a} and R^{b} are independently H or C₁-C₃ alkyl. In some embodiments, R² is C₁-C₃ alkyl substituted with —NR^{a}R^{b}, wherein R^{a} and R^{b} are independently H, C₁-C₃ alkyl, phenyl, —(C₁-C₃ alkylene)-O—(C₁-C₃ alkyl), C₃-C₆ cycloalkyl, —(C₁-C₃ alkylene)-(C₃-C₆ cycloalkyl), S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-O—(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2d}, when present, is independently halo, —OH, C₁-C₃ alkyl, C₁-C₃ hydroxyalkyl, or C₁-C₃ haloalkyl. In some embodiments, R² is C₁-C₃ alkyl substituted with a 4- to 8-membered heterocycloalkyl ring having 1-2 ring heteroatoms independently selected from N, S, and O, and the 4- or 8-membered heterocycloalkyl ring is substituted with 0-3 substituents independently selected from halo, —OH, C₁-C₃ alkyl, C₁-C₃ haloalkyl, and C₁-C₃ hydroxyalkyl. In some embodiments, R² is C₁-C₃ alkyl substituted with a 5- to 6-membered heteroaryl containing 1-3 ring heteroatoms independently selected from N, S, and O.

In some embodiments, R², when present, is H, or C₁-C₃ alkyl substituted with —OH, —NR^{2a}R^{2b}, or 4- to 8-membered heterocycloalkyl ring having 1-2 ring heteroatoms independently selected from N, and O; wherein R^{2a} and R^{2b} are independently selected from H and C₁-C₆ alkyl; and the 4- to 8-membered heterocycloalkyl ring is substituted with 0-3 substituents independently selected from halo, and C₁-C₃ alkyl.

In some embodiments, R² is H; 5- to 6-membered heteroaryl containing 1-3 ring heteroatoms independently selected from N, S, and O, and the 5- or 6-membered heteroaryl is substituted with 0-3 C₁-C₃ alkyl; 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl; or C₁-C₃ alkyl substituted with —OH or a 5- or 6-membered heterocycloalkyl ring optionally having 1-2 ring heteroatoms independently selected from N, S, and O, wherein the 5- or 6-membered heterocycloalkyl ring is substituted with 0-3 C₁-C₃ alkyl.

In some embodiments, R² is H.

In some embodiments, R² is H, C₁-C₃ alkyl substituted with —OH, or —NR^{2a}R^{2b}, wherein R^{2a} and R^{2b} are independently selected from H and C₁-C₆ alkyl.

In some embodiments, R², when present, is C₁-C₃ alkyl optionally substituted with a 4- or 8-membered heterocycloalkyl ring optionally having 1-2 ring heteroatoms independently selected from N, S, and O, wherein the 5- or 6-membered heterocycloalkyl ring is optionally substituted with 1-3 substituents independently selected from —OH, C₁-C₃ alkyl, halo, C₁-C₃ haloalkyl, and C₁-C₃ hydroxyalkyl.

In some embodiments, R², when present, is 5-membered heteroaryl containing 1-3 ring heteroatoms independently selected from N, S, and O, and the 5-membered heteroaryl is optionally substituted with 1-3 substituents independently selected from C₁-C₃ alkyl.

In some embodiments, R², when present, is 6-membered heterocycloalkyl ring having 1 heteroatom independently selected from N, S, and O, and optionally having 1 additional ring heteroatom selected from N, S, and O, and the 6-membered heterocycloalkyl ring is optionally substituted with 1-3 substituents independently selected from C₁-C₃ alkyl.

In some embodiments, R², when present, is C₁-C₃ alkyl optionally substituted with —OH or —NR^{a}R^{b}; and R^{a} and R^{b} are each independently H, C₁-C₃ alkyl, phenyl, —(C₁-C₃ alkylene)-O—(C₁-C₃ alkyl), C₃-C₆ cycloalkyl, —(C₁-C₃ alkylene)-(C₃-C₆ cycloalkyl), S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-O—(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2d}, when present, is independently halo, —OH, C₁-C₃ alkyl, C₁-C₃ hydroxyalkyl, or C₁-C₃ haloalkyl.

In some embodiments, R², when present, is —H,

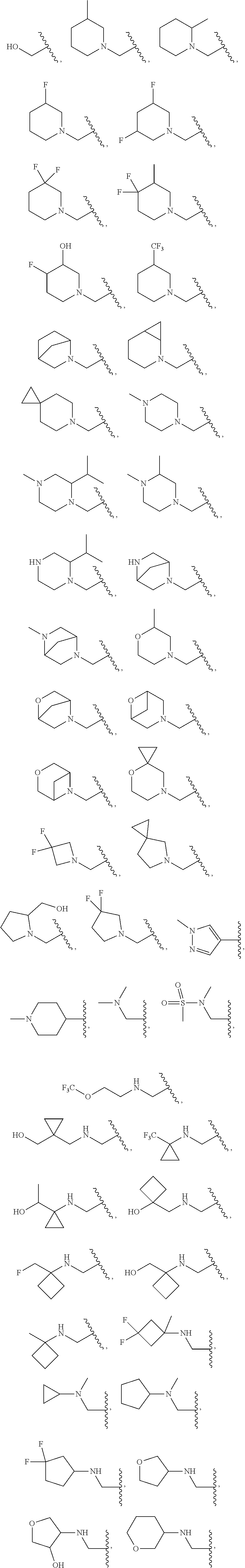

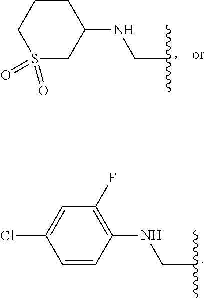 [SMILES: *CNC1CCCS(=O)(=O)C1 ; *CNc1ccc(Cl)cc1F]

In some embodiments, R² is

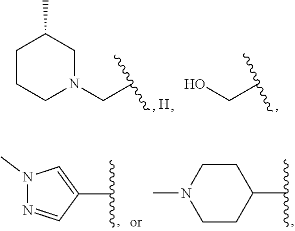

In some embodiments, R³ is H. In some embodiments, R³ is —CN. In some embodiments, R³ is C₁-C₃ alkyl. In some embodiments, R³ is C₁-C₃ alkylene-NRcRd; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl. In some embodiments, R³ is C₁-C₃ haloalkyl. In some embodiments, R³ is C₁-C₃ hydroxyalkyl. In some embodiments, R³ is C₃-C₆ cycloalkyl. In some embodiments, R³ is —S(O)₂(C₁-C₃ alkyl). In some embodiments, R³ is —COR^{c}, wherein R^{c} is H or C₁-C₃ alkyl. In some embodiments, R³ is —COOR^{c}, wherein R^{c} is H or C₁-C₃ alkyl. In some embodiments, R³ is —CONR^{c}R^{d}; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl. In some embodiments, R³ is a 5-membered heteroaryl containing 1-3 ring heteroatoms independently selected from N, O, and S, and the 5-membered heteroaryl is substituted with 0-3 substituents independently selected from halo, OH, CN, C₁-C₃ alkyl, C₁-C₃ haloalkyl, C₁-C₃ hydroxyalkyl, —C(O)NR^{c}R^{d}, —NR^{c}R^{d}, —COR^{c}, —COOR^{c}, —S(O)₂(C₁-C₃ alkyl), —NR^{c}—S(O)₂(C₁-C₃ alkyl), and —S(O)₂—NR^{c}R^{d}, wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl. In some embodiments, R³ is a 5-membered heteroaryl containing 1-3 ring heteroatoms independently selected from N, O, and S, and the 5-membered heteroaryl is substituted with 0-3 substituents independently selected from halo, OH, CN, C₁-C₃ alkyl, C₁-C₃ haloalkyl, C₁-C₃ hydroxyalkyl, —C(O)NR^{c}R^{d}, —NR^{c}R^{d}, —COR^{c}, —COOR^{c}, —S(O)₂(C₁-C₃ alkyl), —NR—S(O)₂(C₁-C₃ alkyl), and —S(O)₂—NR^{c}R^{d}, wherein R^{c} and R^{d} are independently H or C₁-C₃ alkyl.

In some embodiments, R³, when present, is H; —CN; C₁-C₃ alkyl; C₁-C₃ alkylene-NR^{c}R^{d}, C₁-C₃ haloalkyl; C₁-C₃ hydroxyalkyl; C₃-C₆ cycloalkyl; —S(O)₂(C₁-C₃ alkyl); —COR^{c}; —COOR^{c}; or 5-membered heteroaryl having 1-3 ring nitrogen atoms, and the heteroaryl ring is unsubstituted or substituted with C₁-C₃ alkyl; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is optionally substituted with 1-3 substituents independently selected from C₁-C₃ alkyl.

In some embodiments, R³ is H; —CN; C₁-C₃ alkyl; C₁-C₃ alkylene-NR^{c}R^{d}; C₁-C₃ haloalkyl; C₁-C₃ hydroxyalkyl; C₃-C₆ cycloalkyl; —S(O)₂(C₁-C₃ alkyl); —COR^{c}; —COOR^{c}; or 5-membered heteroaryl having 1-3 ring nitrogen atoms, and the 5-membered heteroaryl is substituted with 0-3 alkyl; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl. In some embodiments, R³ is H, —CN, —CH₃, —CF₃,

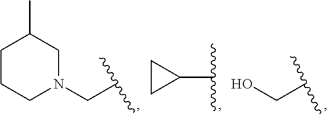

—CO₂H, —COCH₃, —CH(OH)CH₃, —SO₂CH₃,

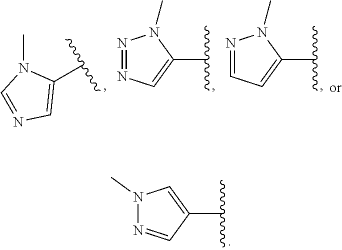

In some embodiments, R³ is H, —CN, —CH₃, —CF₃,

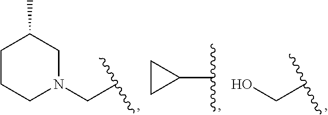

—CO₂H, —COCH₃, —CH(OH)CH₃, —S(O)₂CH₃, or

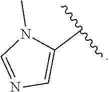 [SMILES: *c1cncn1C]

In some embodiments, R³ is H.

In some embodiments, R¹ is —H. In some embodiments, R¹ is C₁-C₃ alkyl.

In some embodiments, R¹ and R³ are H.

In some embodiments, ring A is

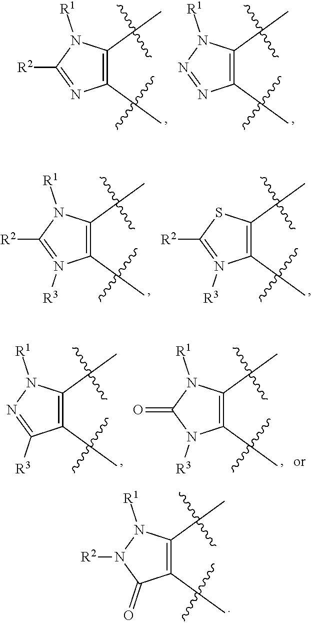

In some embodiments, ring A is

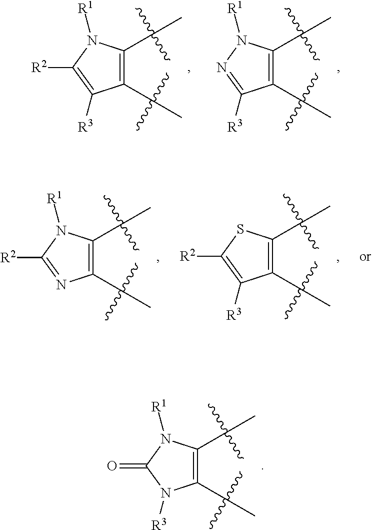

In some embodiments, ring A is

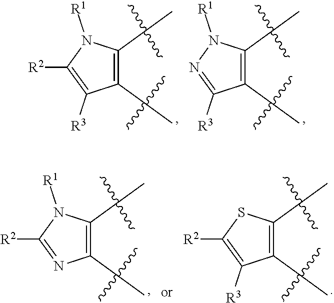

In some embodiments, ring A is

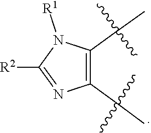

In some embodiments, ring A is

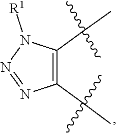

In some embodiments, ring A is

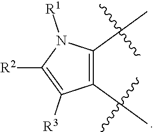

In some embodiments, ring A is

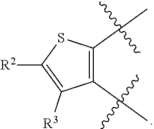

In some embodiments, ring A is

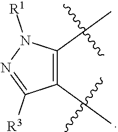

In some embodiments, ring A is

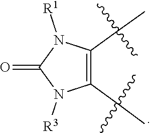

In some embodiments, ring A is

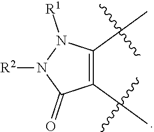

In some embodiments, ring A is

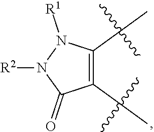

and R² is not CN or NR^{2a}R^{2b}.

In some embodiments, X¹ is CR⁴. In some embodiments, X¹ is N.

In some embodiments, R⁴ is H. In some embodiments, R⁴ is —CN. In some embodiments, R⁴ is halogen. In some embodiments, R⁴ is C₁-C₃ alkyl. In some embodiments, R⁴ is C₁-C₃ haloalkyl. In some embodiments, R⁴ is C₁-C₃ hydroxyalkyl. In some embodiments, R⁴ is C₂-C₃ alkenyl. In some embodiments, R⁴ is C₃-C₄ cycloalkyl. In some embodiments, R⁴ is —S(O)₂(C₁-C₃ alkyl). In some embodiments, R⁴ is —C(O)OH. In some embodiments, R⁴ is 5- or 6-membered heteroaryl having 1 to 4 ring heteroatoms independently selected from N, S, and O, and the heteroaryl is substituted with 0-3 C₁-C₃ alkyl.

In some embodiments, R⁴ is C₃-C₄ cycloalkyl, H, CN, C₁-C₃ alkyl, C₁-C₃ haloalkyl, C₁-C₃ hydroxyalkyl, —S(O)₂(C₁-C₃ alkyl), or —C(O)OH. In some embodiments R⁴ is H, CN, methyl, —CF₃, cyclopropyl, —S(O)₂CH₃, —C(O)OH, or —CH₂OH.

In some embodiments, R⁴ is C₃-C₄ cycloalkyl, H, C₁-C₃ alkyl, C₁-C₃ haloalkyl, or —CN. In some embodiments, R⁴ is cyclopropyl, H, methyl, —CF₃, or —CN.

In some embodiments, X² is N or CR⁵. In some embodiments, X² is N. In some embodiments, X² is CR⁵.

In some embodiments, R⁵ is H. In some embodiments, R⁵ is C₁-C₃ alkyl.

In some embodiments, R¹, R³, and R⁵ are H.

In some embodiments, ring B is phenylene. In some embodiments, ring B is 5- to 6-membered heteroarylene having 1-3 ring heteroatoms independently selected from N, S, and O. In some embodiments, ring B is pyridinylene. In some embodiments, ring B is phenylene or pyridinylene.

In some embodiments, J is —(CR^{g}R^{h})—Y¹—; wherein Y¹ is absent, CH₂, or S; and each R^{g} and each R^{h} is independently H, halo, —OH, C₁-C₃ alkyl, C₁-C₃ haloalkyl, C₁-C₃ alkoxy, or C₃-C₄ cycloalkyl; or R^{g} and R^{h} together with the carbon atom to which they are attached form a 3- to 4-membered heterocycle ring having 1 ring oxygen atom, C₃-C₄ cycloalkyl ring, or a C₆-C₈ spirocyclic ring, where each cycloalkyl ring or spirocyclic ring is optionally substituted with 1-3 R^{j}; and each R^{j} is independently —OH, halogen, C₁-C₃ alkyl, C₁-C₃ alkoxy, and C₁-C₃ haloalkoxy.

In some embodiments, J is —(CR^{g}R^{h})ₙ—Y¹— or —Y¹—(CR^{g}R^{h})ₙ—; wherein n is 1 or 2; Y¹ is absent, CH₂, CHF, CF₂, O, S, S(O), or S(O)₂; and each R^{g} and each R^{h} is independently H, C₁-C₃ alkyl, or C₃-C₄ cycloalkyl; or R^{g} and R^{h} together with the carbon atom to which they are attached form a 3- to 4-membered heterocycle ring having 1 ring heteroatom selected from N, O, and S, C₃-C₄ cycloalkyl ring, or a C₆-C₈ spirocyclic ring, where each cycloalkyl ring or spirocyclic ring is substituted with 0-3 R^{j}; and each R^{j} is independently selected from —CN, —OH, halogen, C₁-C₃ alkyl, C₁-C₃ alkoxy, and C₁-C₃ haloalkoxy. In some embodiments, J is —(CR^{g}R^{h})—Y¹—; wherein Y¹ is absent, CH₂, or S; and each R^{g} and each R^{h} is independently H, C₁-C₃ alkyl, or C₃-C₄ cycloalkyl; or R^{g} and R^{h} together with the carbon atom to which they are attached form a 3- to 4-membered heterocycle ring having 1 ring oxygen atom, C₃-C₄ cycloalkyl ring, or a C₆-C₈ spirocyclic ring, where each cycloalkyl ring or spirocyclic ring is optionally substituted with 1-3 R^{j}; and each R^{j} is independently —OH, halogen, C₁-C₃ alkyl, C₁-C₃ alkoxy, and C₁-C₃ haloalkoxy.

In some embodiments, J is —(CR^{g}R^{h})ₙ—Y¹—; wherein n is 1 or 2; Y¹ is absent, CH₂, CHF, CF₂, O, S, S(O), or S(O)₂; and each R^{g} and each R^{h} is independently H or C₁-C₃ alkyl; or R^{g} and R^{h} together with the carbon atom to which they are attached form a C₃-C₄ cycloalkyl ring or a C₆-C₈ spirocyclic ring, where each cycloalkyl ring or spirocyclic ring is substituted with 0-3 R^{d}; and each R^{j} is independently selected from —CN, halogen, C₁-C₃ alkyl, C₁-C₃ alkoxy, and C₁-C₃ haloalkoxy. In some embodiments, J is —(CR^{g}R^{h})—Y¹—; wherein Y¹ is absent or S; and each R^{g} and each R^{h} is independently H or C₁-C₃ alkyl; or R^{g} and R^{h} together with the carbon atom to which they are attached form a C₃-C₄ cycloalkyl ring or a C₆-C₈ spirocyclic ring, where each cycloalkyl ring or spirocyclic ring is substituted with 0-3 R^{j}; and each R^{j} is independently C₁-C₃ alkyl.

In some embodiments, J is —Y¹—(CR^{g}R^{h})ₙ—; wherein n is 1 or 2; Y¹ is absent, CH₂, CHF, CF₂, O, S, S(O), or S(O)₂; and each R^{g} and each R^{h} is independently H or C₁-C₃ alkyl; or R^{g} and R^{h} together with the carbon atom to which they are attached form a C₃-C₄ cycloalkyl ring or a C₆-C₈ spirocyclic ring, where each cycloalkyl ring or spirocyclic ring is substituted with 0-3 R^{j}; and each R^{j} is independently selected from —CN, halogen, C₁-C₃ alkyl, C₁-C₃ alkoxy, and C₁-C₃ haloalkoxy.

In some embodiments, J is —(CR^{g}R^{h})—, and R^{g} and R^{h} together with the carbon atom to which they are attached form a C₃-C₄ cycloalkyl ring or a C₆-C₈ spirocyclic ring, where each cycloalkyl ring or spirocyclic ring is optionally substituted with 1-3 R^{j}; and each R^{j} is independently selected from —OH, halogen, C₁-C₃ alkyl, C₁-C₃ alkoxy, and C₁-C₃ haloalkoxy. In some embodiments, J is —(CR^{g}R^{h})—, and R^{g} and R^{h} together with the carbon atom to which they are attached form a C₃-C₄ cycloalkyl ring or a C₆-C₈ spirocyclic ring, where each cycloalkyl ring or spirocyclic ring is substituted with 0-3 R^{j}; and each R^{j} is independently selected from —CN, halogen, C₁-C₃ alkyl, C₁-C₃ alkoxy, and C₁-C₃ haloalkoxy.

In some embodiments, J is —(CR^{g}R^{h})—, and R^{g} and R^{h} together with the carbon atom to which they are attached form cyclobutyl, and said cyclobutyl is substituted with 0-2 R^{j} independently selected from —OH, halogen, C₁-C₃ alkyl, C₁-C₃ alkoxy, and C₁-C₃ haloalkoxy. In some embodiments, J is —(CR^{g}R^{h})—, and R^{g} and R^{h} together with the carbon atoms to which they are attached form a spiro[2.3]hexane. In some embodiments, J is —Y¹—(CR^{g}R^{h})ₙ—; wherein n is 1; Y¹ is CH₂; and R^{g} and R^{h} together with the carbon atom to which they are attached form oxetane. In some embodiments, J is —Y¹—(CR^{g}R^{h})ₙ; n is 1, Y¹ is S; and R^{g} and R^{h} are independently H or C₁-C₃ alkyl.

In some embodiments, J is —(CR^{g}R^{h})ₙ—Y¹—; wherein Y¹ is absent, CH₂, or S; and each R^{g} and R^{h} is independently H, fluoro, —OH, —CH₃, —CF₃, —OCH₃, or cyclobutyl; or R^{g} and R^{h} together with the carbon atom to which they are attached form cyclobutyl, oxetanyl, or spiro[2.3]hexane, each of which is optionally substituted with 1-3 R^{j}; and each R^{j} is independently fluoro, —OH, —CH₃, —OCH₃, —OCH₂CH₃, or —OCF₂H.

In some embodiments, J is

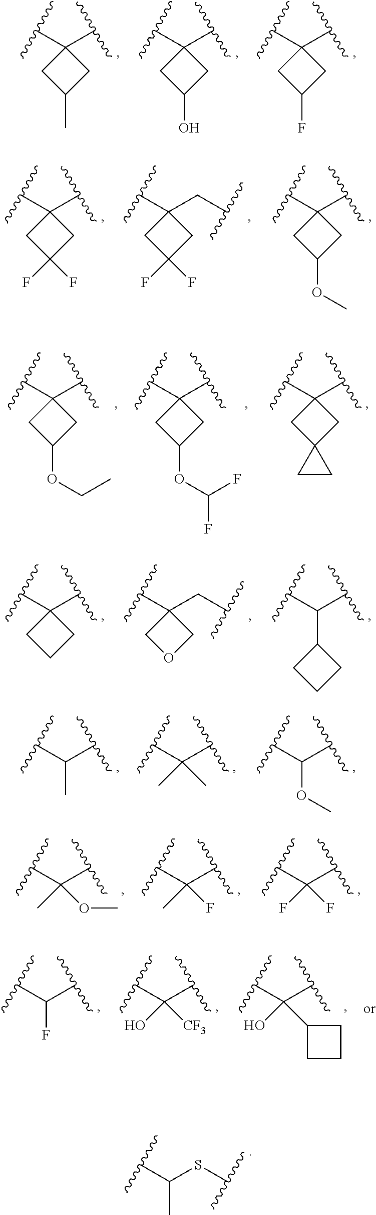

In some embodiments, J is

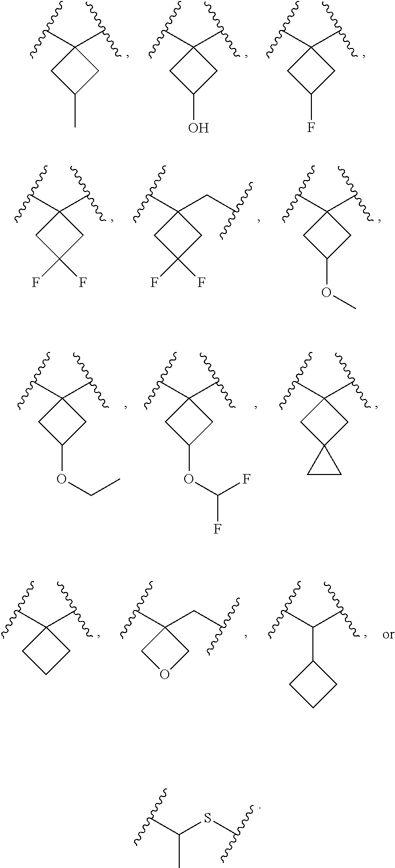

In some embodiments, J is

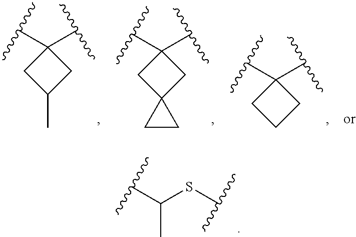

In some embodiments, J is

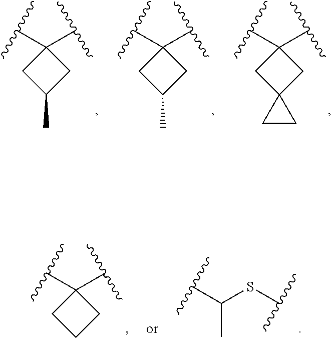

In some embodiments, ring C is 5- to 6-membered heteroaryl containing 1-3 ring heteroatoms independently selected from N, S, and O, and the 5- to 6-membered heteroaryl is substituted with 0-3 substituents independently selected from C₁-C₃ alkyl, C₁-C₃ haloalkyl, and C₃-C₄ cycloalkyl. In some embodiments, ring C is a 6-membered heteroaryl containing 1-3 ring heteroatoms independently selected from N, S, and O, and the 6-membered heteroaryl is substituted with 0-3 substituents independently selected from C₁-C₃ alkyl, C₁-C₃ haloalkyl, and C₃-C₄ cycloalkyl. In some embodiments, ring C is a 5-membered heteroaryl containing 1-3 ring heteroatoms independently selected from N, S, and O, and the 5-membered heteroaryl is substituted with 0-3 substituents independently selected from C₁-C₃ alkyl, C₁-C₃ haloalkyl, and C₃-C₄ cycloalkyl. In some embodiments, ring C is a 5-membered heteroaryl containing 1-3 ring heteroatoms independently selected from O and N, and the 5-membered heteroaryl is substituted with 0-3 substituents independently selected from C₁-C₃ alkyl, C₁-C₃ haloalkyl, and C₃-C₄ cycloalkyl. In some embodiments, ring C is imidazolyl, pyrazolyl, oxazolyl, oxadiazolyl, or triazolyl, each of which is substituted with 0-3 C₁-C₃ alkyl C₁-C₃ haloalkyl, and C₃-C₆ cycloalkyl.

In some embodiments, ring C is

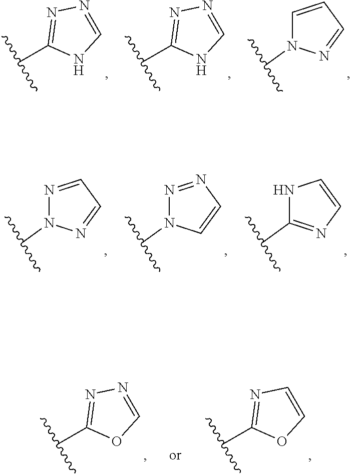

each of which is substituted with 0-3 substituents independently selected from C₁-C₃ alkyl, C₁-C₃ haloalkyl, and C₃-C₄ cycloalkyl. In some embodiments, ring C is substituted with 0-3 substituents independently selected from —CH₃, —CF₂H, —CF₃, —CH₂CF₂CH₂CF₃, —CH(CH₃)₂, and cyclopropyl. In some embodiments, ring C is

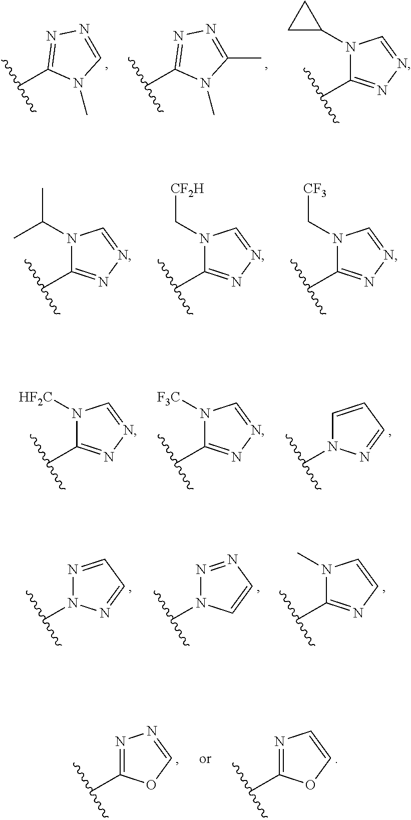

In some embodiments, ring C is 5-membered heteroaryl containing 1-3 ring heteroatoms independently selected from N, S, and O, and the 5-membered heteroaryl is substituted with 0-3 C₁-C₃ alkyl. In some embodiments, ring C is

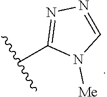 [SMILES: *c1nncn1C]

In one or more embodiments, the disclosure provides compounds having a structure according to Formula I:

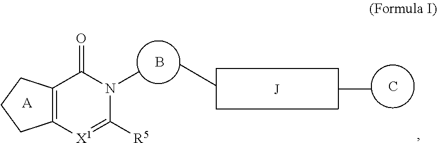

or a pharmaceutically acceptable salt thereof, wherein ring A, X¹, R⁵, B, J, and C have the meanings described herein.

In some embodiments, this disclosure provides a compound having a structure according to Formula I:

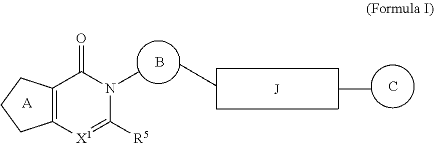

or a pharmaceutically acceptable salt thereof;
wherein:
ring A is selected from the group consisting of:

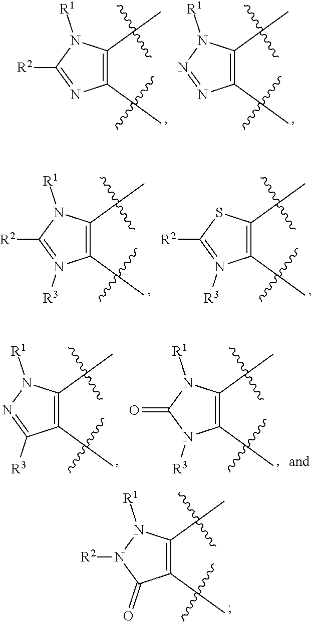

R¹, when present, is —H or C₁-C₃ alkyl;
R², when present, is H; —CN; —NR^{2a}R^{2b}, wherein R^{2a} and R^{2b} are independently selected from H and C₁-C₆ alkyl; —S(O)₂—C₁-C₃ alkyl; —S(O)(NR^{2c})—C₁-C₃ alkyl; —NR^{2c}—S(O)₂—C₁-C₃ alkyl; —S(O)₂—N(R^{2c})₂; 5- to 6-membered heteroaryl containing 1-3 ring heteroatoms independently selected from N, S, and O, and the 5- or 6-membered heteroaryl is substituted with 0-3 C₁-C₃ alkyl; 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl; unsubstituted C₁-C₃ alkyl; or C₁-C₃ alkyl substituted with (i) —OR^{2c}, (ii) —C(O)NR^{a}R^{b}, (iii) —NR^{a}R^{b}, (iv) a 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl, or (v) 5- to 6-membered heteroaryl containing 1-3 ring heteroatoms independently selected from N, S, and O; wherein R², is H or C₁-C₃ alkyl; and R^{a} and R^{b} are independently H or C₁-C₃ alkyl;
R³, when present, is H; —CN; C₁-C₃ alkyl; C₁-C₃ alkylene-NR^{c}R^{d}; C₁-C₃ haloalkyl; C₁-C₃ hydroxyalkyl; C₃-C₆ cycloalkyl; —S(O)₂(C₁-C₃ alkyl); —COR^{c}; —COOR^{c}; —CONR^{c}R^{d}; or 5-membered heteroaryl containing 1-3 ring heteroatoms independently selected from N, O, and S, and the 5-membered heteroaryl is substituted with 0-3 substituents independently selected from halo, OH, CN, C₁-C₃ alkyl, C₁-C₃ haloalkyl, C₁-C₃ hydroxyalkyl, —C(O)NR^{c}R^{d}, —NR^{c}R^{d}, —COR^{c}, —COOR^{c}, —S(O)₂(C₁-C₃ alkyl), —N—S(O)₂(C₁-C₃ alkyl), and —S(O)₂—NR^{c}R^{d}; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl;
X¹ is CR⁴ or N;
R⁴, when present, is H; —CN; halogen; C₁-C₃ alkyl; C₁-C₃ haloalkyl; C₂-C₃ alkenyl; C₃-C₄ cycloalkyl; 5- or 6-membered heteroaryl having 1 to 4 ring heteroatoms independently selected from N, S, and O, and the heteroaryl is substituted with 0-3 C₁-C₃ alkyl;
R⁵ is H or C₁-C₃ alkyl;
ring B is phenylene, or 5- to 6-membered heteroarylene having 1-3 ring heteroatoms independently selected from N, S, and O;
J is —(CR^{g}R^{h})ₙ—Y¹— or —Y¹—(CR^{g}R^{h})ₙ—; wherein n is 1 or 2; Y¹ is absent, CH₂, CHF, CF₂, O, S, S(O), or S(O)₂; and each R^{g} and each R^{h} is independently H or C₁-C₃ alkyl; or R^{g} and R^{h} together with the carbon atom to which they are attached form a C₃-C₄ cycloalkyl ring or a C₆-C₈ spirocyclic ring, where each cycloalkyl ring or spirocyclic ring is substituted with 0-3 R^{j}; and each R^{j} is independently selected from —CN, halogen, C₁-C₃ alkyl, C₁-C₃ alkoxy, and C₁-C₃ haloalkoxy; and ring C is 5- to 6-membered heteroaryl containing 1-3 ring heteroatoms independently selected from N, S, and O, and the 5- to 6-membered heteroaryl is substituted with 0-3 C₁-C₃ alkyl.

In some embodiments, R¹, when present, is H.

In some embodiments, R², when present, is H; 5- to 6-membered heteroaryl containing 1-3 ring heteroatoms independently selected from N, S, and O, and the 5- or 6-membered heteroaryl is substituted with 0-3 C₁-C₃ alkyl; 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl; or C₁-C₃ alkyl substituted with —OH or a 5- or 6-membered heterocycloalkyl ring having 1-2 ring heteroatoms independently selected from N, S, and O, wherein the 5- or 6-membered heterocycloalkyl ring is substituted with 0-3 C₁-C₃ alkyl.

In some embodiments, R², when present, is H,

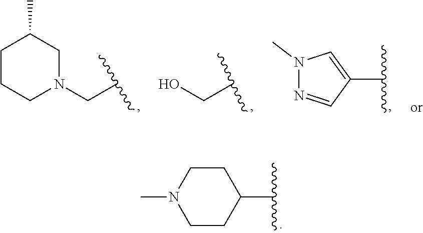

In some embodiments, R², when present, is H.

In some embodiments, R³, when present, is H; —CN; C₁-C₃ alkyl; C₁-C₃alkylene-NR^{c}R^{d}. C₁-C₃ haloalkyl; C₁-C₃ hydroxyalkyl; C₃-C₆ cycloalkyl; —S(O)₂(C₁-C₃ alkyl); —COR^{c}; —COOR^{c}; or 5-membered heteroaryl having 1-3 ring nitrogen atoms, and the 5-membered heteroaryl is substituted with 0-3 alkyl; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl.

In some embodiments, R³, when present, is H, —CN, —CH₃, —CF₃,

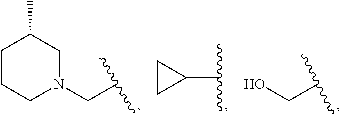

—CO₂H, —COCH₃, —CH(OH)CH₃, —S(O)₂CH₃, or

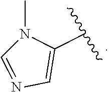 [SMILES: *c1cncn1C]

In some embodiments, R³, when present, is H.

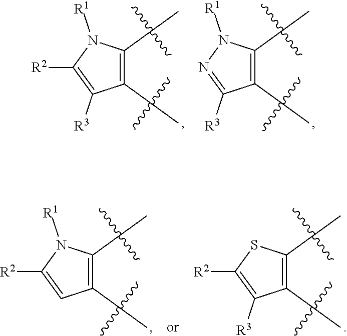

In some embodiments, ring A is

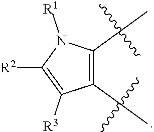

In some embodiments, ring A is

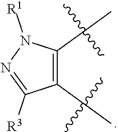

In some embodiments, ring A is

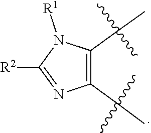

In some embodiments, ring A is

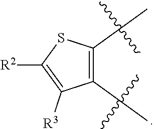

In some embodiments, X¹ is CR⁴.

In some embodiments, R⁴ is C₃-C₄ cycloalkyl, H, C₁-C₃ alkyl, C₁-C₃ haloalkyl, or —CN.

In some embodiments, R⁴ is cyclopropyl, H, methyl, —CF₃, or —CN.

In some embodiments, R⁵ is H.

In some embodiments, ring B is phenylene.

In some embodiments, J is —(CR^{g}R^{h})—Y¹—; wherein Y¹ is absent or S; and each R^{g} and each R^{h} is independently H or C₁-C₃ alkyl; or R^{g} and R^{h} together with the carbon atom to which they are attached form a C₃-C₄ cycloalkyl ring or a C₆-C₈ spirocyclic ring, where each cycloalkyl ring or spirocyclic ring is substituted with 0-3 R^{j}; and each R^{j} is independently C₁-C₃ alkyl.

In some embodiments, J is —(CR^{g}R^{h})—, and R^{g} and R^{h} together with the carbon atom to which they are attached form a C₃-C₄ cycloalkyl ring or a C₆-C₈ spirocyclic ring, where each cycloalkyl ring or spirocyclic ring is substituted with 0-3 R^{j}; and each R is independently selected from —CN, halogen, C₁-C₃ alkyl, C₁-C₃ alkoxy, and C₁-C₃ haloalkoxy.

In some embodiments, J is

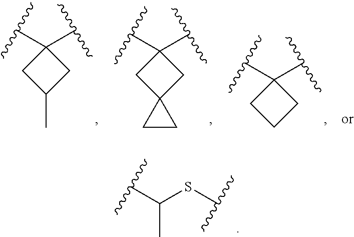

In one or more embodiments, the disclosure provides a compound having a structure selected from the group consisting of:

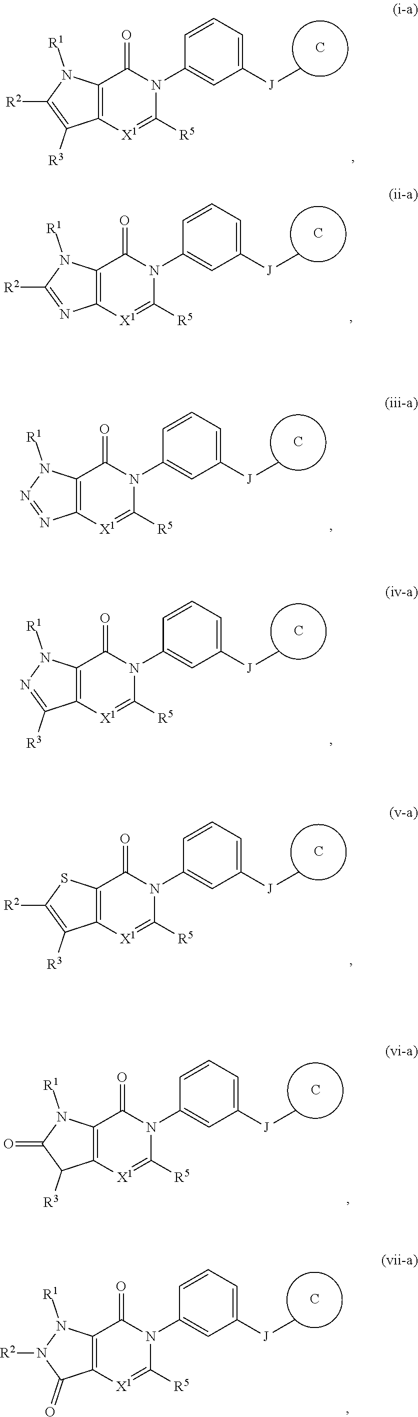

wherein R¹, R², R³, X¹, R⁵, ring C and J have the meanings provided above.

In one or more embodiments, the disclosure provides a compound having a structure selected from the group consisting of:

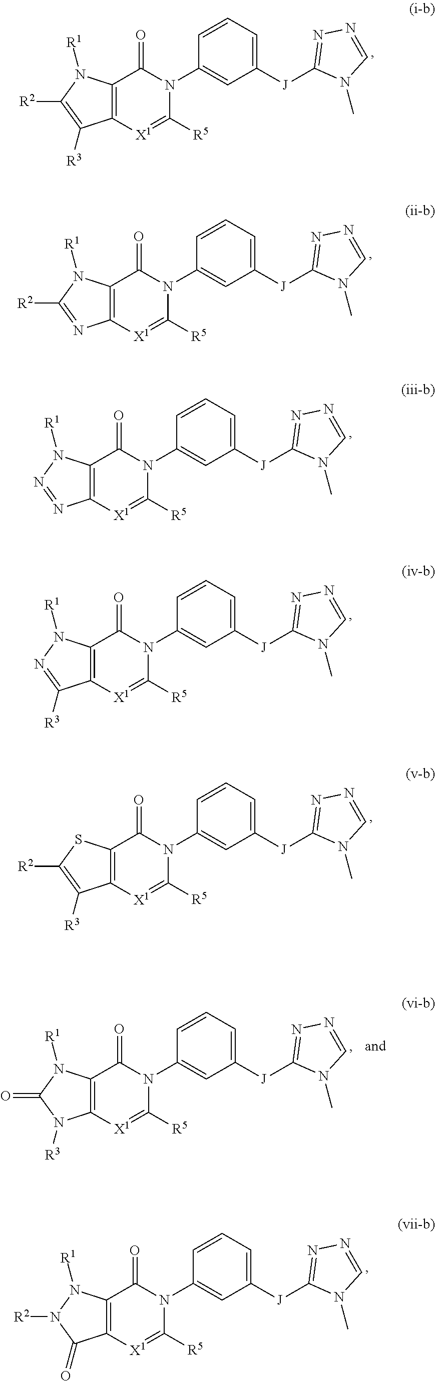

wherein R¹, R², R³, X¹, R⁵, and J have the meanings provided above.

In one or more embodiments, the compound, or pharmaceutically acceptable salt or solvate thereof, according to this disclosure is selected from the compounds provided in Table 1 or Table 2. In another embodiment, the compound, or pharmaceutically acceptable salt thereof, according to this disclosure is selected from the compounds provided in Table 1 or Table 2. In another embodiment, the compound is selected from the compounds provided in Table 1 or Table 2.

TABLE 1

| Example | Structure | Example | Structure |
|---|---|---|---|
| 1 | 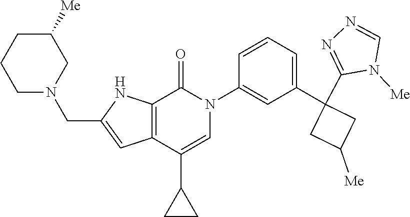 [SMILES: CC1CC(c2cccc(-n3cc(C4CC4)c4cc(CN5CCC[C@H](C)C5)[nH]c4c3=O)c2)(c2nncn2C)C1] | 2 | 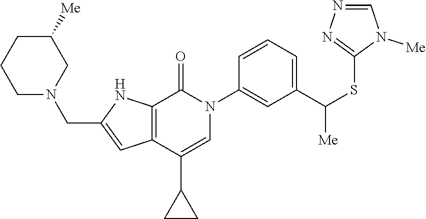 [SMILES: CC(Sc1nncn1C)c1cccc(-n2cc(C3CC3)c3cc(CN4CCC[C@H](C)C4)[nH]c3c2=O)c1] |
| 3 | 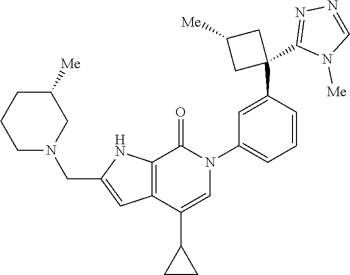 | 4 | 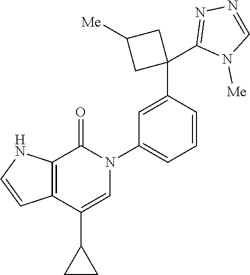 [SMILES: CC1CC(c2cccc(-n3cc(C4CC4)c4cc[nH]c4c3=O)c2)(c2nncn2C)C1] |
| 5 | 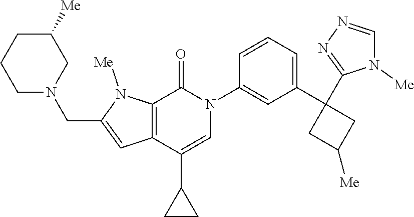 [SMILES: CC1CC(c2cccc(-n3cc(C4CC4)c4cc(CN5CCC[C@H](C)C5)n(C)c4c3=O)c2)(c2nncn2C)C1] | 6 | 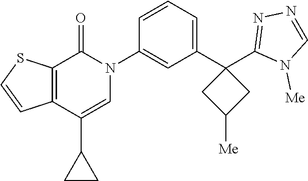 [SMILES: CC1CC(c2cccc(-n3cc(C4CC4)c4ccsc4c3=O)c2)(c2nncn2C)C1] |
| 7 | 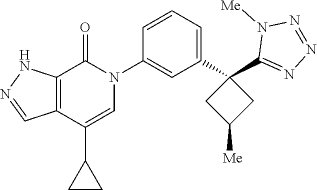 [SMILES: Cn1nnnc1[C@]1(c2cccc(-n3cc(C4CC4)c4cn[nH]c4c3=O)c2)C[C@H](C)C1] | 8 | 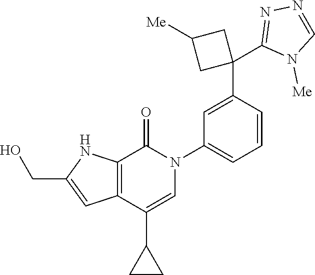 [SMILES: CC1CC(c2cccc(-n3cc(C4CC4)c4cc(CO)[nH]c4c3=O)c2)(c2nncn2C)C1] |
| 9 | 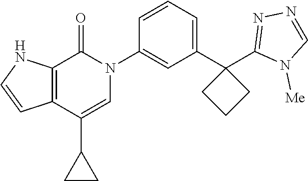 [SMILES: Cn1cnnc1C1(c2cccc(-n3cc(C4CC4)c4cc[nH]c4c3=O)c2)CCC1] | 10 | 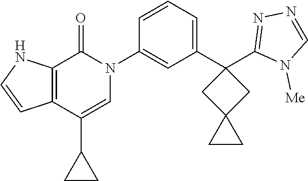 [SMILES: Cn1cnnc1C1(c2cccc(-n3cc(C4CC4)c4cc[nH]c4c3=O)c2)CC2(CC2)C1] |
| 11 | 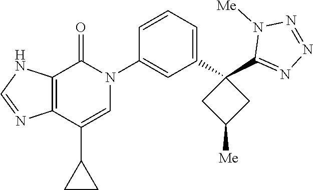 [SMILES: Cn1nnnc1[C@]1(c2cccc(-n3cc(C4CC4)c4nc[nH]c4c3=O)c2)C[C@H](C)C1] | 12 | 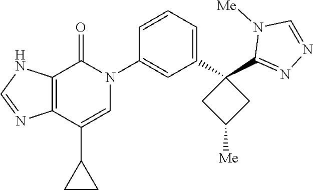 [SMILES: Cn1cnnc1[C@]1(c2cccc(-n3cc(C4CC4)c4nc[nH]c4c3=O)c2)C[C@@H](C)C1] |
| 13 | 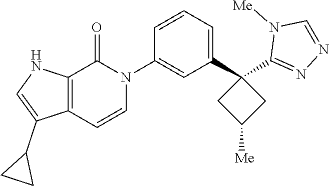 [SMILES: Cn1cnnc1[C@]1(c2cccc(-n3ccc4c(C5CC5)c[nH]c4c3=O)c2)C[C@H](C)C1] | 14 | 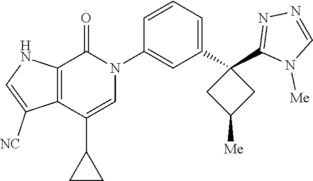 [SMILES: Cn1cnnc1[C@]1(c2cccc(-n3cc(C4CC4)c4c(C#N)c[nH]c4c3=O)c2)C[C@H](C)C1] |
| 15 | 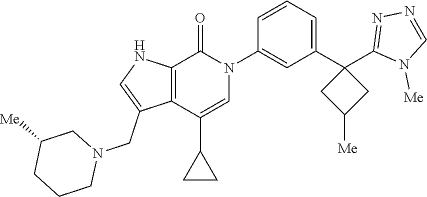 [SMILES: CC1CC(c2cccc(-n3cc(C4CC4)c4c(CN5CCC[C@H](C)C5)c[nH]c4c3=O)c2)(c2nncn2C)C1] | 16 | 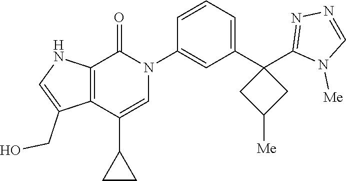 [SMILES: CC1CC(c2cccc(-n3cc(C4CC4)c4c(CO)c[nH]c4c3=O)c2)(c2nncn2C)C1] |
| 17 | 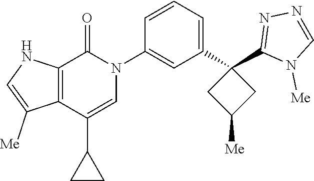 [SMILES: Cc1c[nH]c2c(=O)n(-c3cccc([C@]4(c5nncn5C)C[C@@H](C)C4)c3)cc(C3CC3)c12] | 18 | 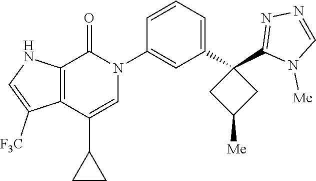 [SMILES: Cn1cnnc1[C@]1(c2cccc(-n3cc(C4CC4)c4c(C(F)(F)F)c[nH]c4c3=O)c2)C[C@H](C)C1] |
| 19 | 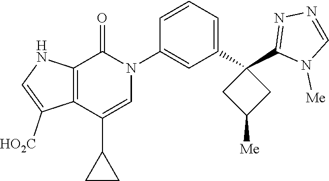 [SMILES: Cn1cnnc1[C@]1(c2cccc(-n3cc(C4CC4)c4c(C(=O)O)c[nH]c4c3=O)c2)C[C@H](C)C1] | 20 | 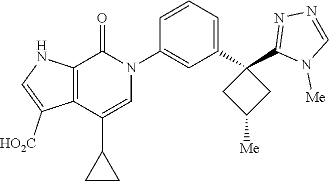 [SMILES: Cn1cnnc1[C@]1(c2cccc(-n3cc(C4CC4)c4c(C(=O)O)c[nH]c4c3=O)c2)C[C@@H](C)C1] |
| 21 | 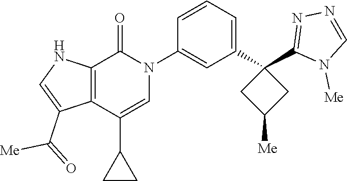 [SMILES: CC(=O)c1c[nH]c2c(=O)n(-c3cccc([C@]4(c5nncn5C)C[C@@H](C)C4)c3)cc(C3CC3)c12] | 22 | 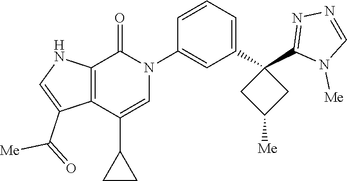 [SMILES: CC(=O)c1c[nH]c2c(=O)n(-c3cccc([C@]4(c5nncn5C)C[C@H](C)C4)c3)cc(C3CC3)c12] |
| 23 | 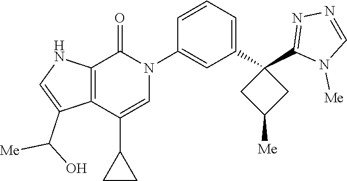 [SMILES: CC(O)c1c[nH]c2c(=O)n(-c3cccc([C@]4(c5nncn5C)C[C@@H](C)C4)c3)cc(C3CC3)c12] | 24 | 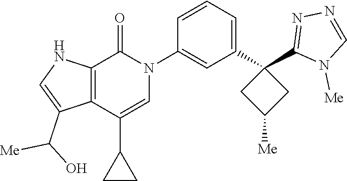 [SMILES: CC(O)c1c[nH]c2c(=O)n(-c3cccc([C@]4(c5nncn5C)C[C@H](C)C4)c3)cc(C3CC3)c12] |
| 25 | 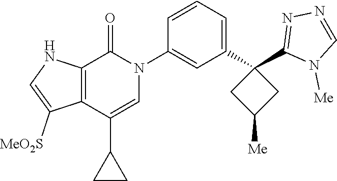 [SMILES: Cn1cnnc1[C@]1(c2cccc(-n3cc(C4CC4)c4c(S(C)(=O)=O)c[nH]c4c3=O)c2)C[C@H](C)C1] | 26 | 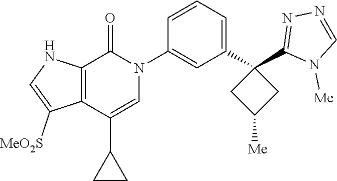 [SMILES: Cn1cnnc1[C@]1(c2cccc(-n3cc(C4CC4)c4c(S(C)(=O)=O)c[nH]c4c3=O)c2)C[C@@H](C)C1] |
| 27 | 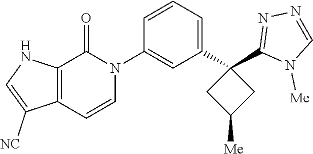 [SMILES: Cn1cnnc1[C@]1(c2cccc(-n3ccc4c(C#N)c[nH]c4c3=O)c2)C[C@H](C)C1] | 28 | 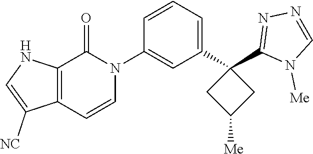 [SMILES: Cn1cnnc1[C@]1(c2cccc(-n3ccc4c(C#N)c[nH]c4c3=O)c2)C[C@@H](C)C1] |
| 29 | 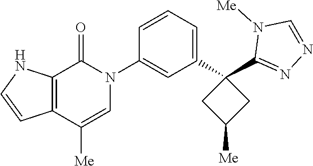 [SMILES: Cc1cn(-c2cccc([C@]3(c4nncn4C)C[C@@H](C)C3)c2)c(=O)c2[nH]ccc12] | 30 | 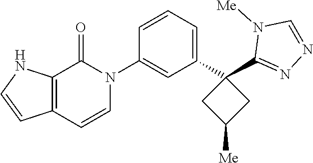 |
| 31 | 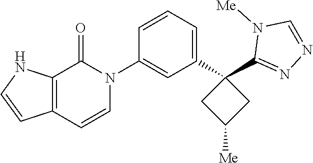 [SMILES: Cn1cnnc1[C@]1(c2cccc(-n3ccc4cc[nH]c4c3=O)c2)C[C@@H](C)C1] | 32 | 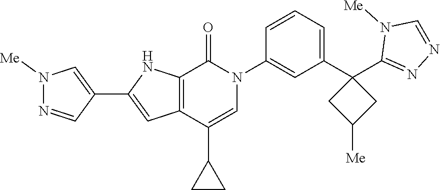 [SMILES: CC1CC(c2cccc(-n3cc(C4CC4)c4cc(-c5cnn(C)c5)[nH]c4c3=O)c2)(c2nncn2C)C1] |
| 33 | 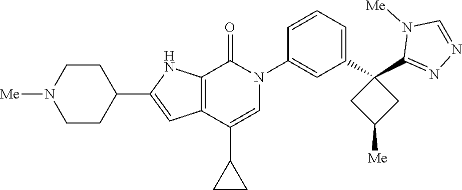 [SMILES: CN1CCC(c2cc3c(C4CC4)cn(-c4cccc([C@]5(c6nncn6C)C[C@@H](C)C5)c4)c(=O)c3[nH]2)CC1] | 34 | 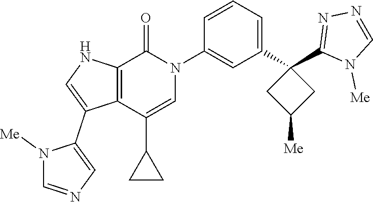 [SMILES: Cn1cncc1-c1c[nH]c2c(=O)n(-c3cccc([C@]4(c5nncn5C)C[C@@H](C)C4)c3)cc(C3CC3)c12] |
| 35 | 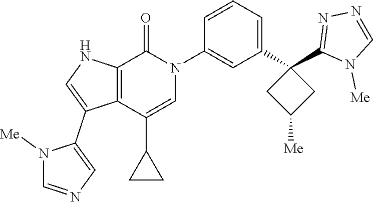 [SMILES: Cn1cncc1-c1c[nH]c2c(=O)n(-c3cccc([C@]4(c5nncn5C)C[C@H](C)C4)c3)cc(C3CC3)c12] | 36 | 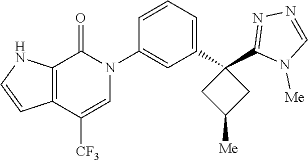 [SMILES: Cn1cnnc1[C@]1(c2cccc(-n3cc(C(F)(F)F)c4cc[nH]c4c3=O)c2)C[C@H](C)C1] |
| 37 | 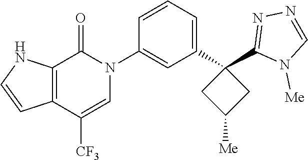 [SMILES: Cn1cnnc1[C@]1(c2cccc(-n3cc(C(F)(F)F)c4cc[nH]c4c3=O)c2)C[C@@H](C)C1] | 38 | 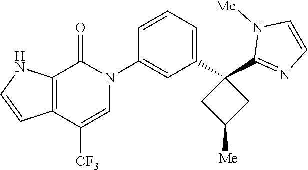 [SMILES: Cn1ccnc1[C@]1(c2cccc(-n3cc(C(F)(F)F)c4cc[nH]c4c3=O)c2)C[C@H](C)C1] |

TABLE 2

| Ex. No. | Structure | Ex. No. | Structure |
|---|---|---|---|
| 39 | 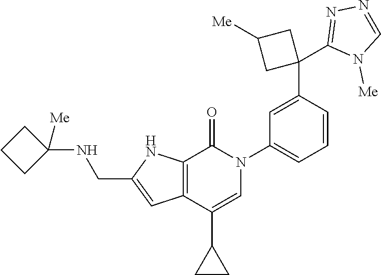 [SMILES: CC1CC(c2cccc(-n3cc(C4CC4)c4cc(CNC5(C)CCC5)[nH]c4c3=O)c2)(c2nncn2C)C1] | 40 | 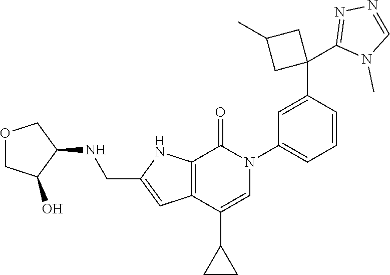 [SMILES: CC1CC(c2cccc(-n3cc(C4CC4)c4cc(CN[C@@H]5COC[C@@H]5O)[nH]c4c3=O)c2)(c2nncn2C)C1] |
| 41 | 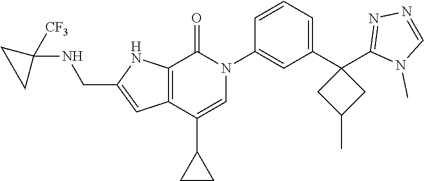 [SMILES: CC1CC(c2cccc(-n3cc(C4CC4)c4cc(CNC5(C(F)(F)F)CC5)[nH]c4c3=O)c2)(c2nncn2C)C1] | 42 | 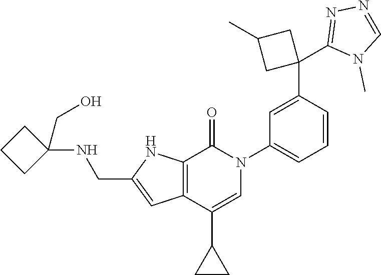 [SMILES: CC1CC(c2cccc(-n3cc(C4CC4)c4cc(CNC5(CO)CCC5)[nH]c4c3=O)c2)(c2nncn2C)C1] |
| 43 | 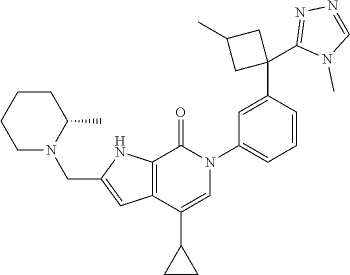 [SMILES: CC1CC(c2cccc(-n3cc(C4CC4)c4cc(CN5CCCC[C@@H]5C)[nH]c4c3=O)c2)(c2nncn2C)C1] | 44 | 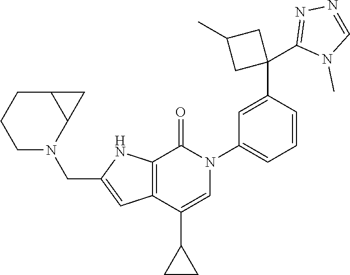 [SMILES: CC1CC(c2cccc(-n3cc(C4CC4)c4cc(CN5CCCC6CC65)[nH]c4c3=O)c2)(c2nncn2C)C1] |
| 45 | 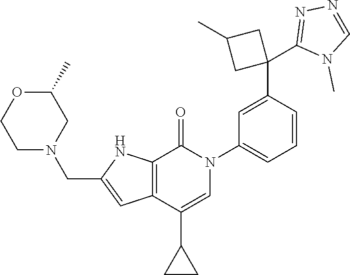 [SMILES: CC1CC(c2cccc(-n3cc(C4CC4)c4cc(CN5CCO[C@H](C)C5)[nH]c4c3=O)c2)(c2nncn2C)C1] | 46 | 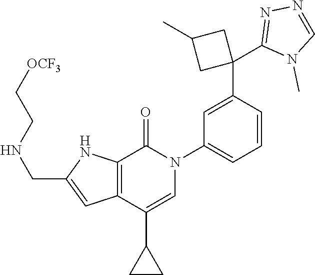 [SMILES: CC1CC(c2cccc(-n3cc(C4CC4)c4cc(CNCCOC(F)(F)F)[nH]c4c3=O)c2)(c2nncn2C)C1] |
| 47 | 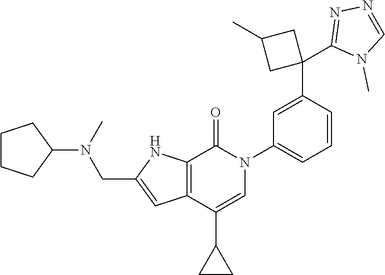 [SMILES: CC1CC(c2cccc(-n3cc(C4CC4)c4cc(CN(C)C5CCCC5)[nH]c4c3=O)c2)(c2nncn2C)C1] | 48 | 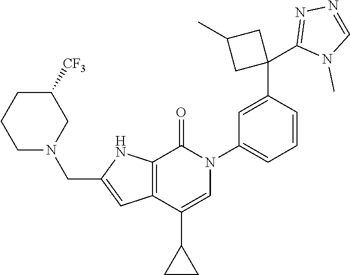 [SMILES: CC1CC(c2cccc(-n3cc(C4CC4)c4cc(CN5CCC[C@H](C(F)(F)F)C5)[nH]c4c3=O)c2)(c2nncn2C)C1] |
| 49 | 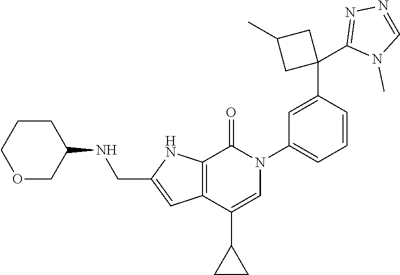 | 50 | 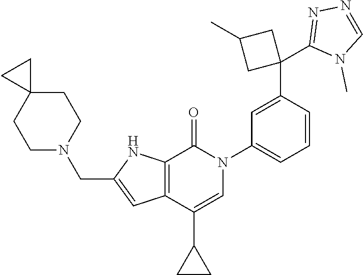 [SMILES: CC1CC(c2cccc(-n3cc(C4CC4)c4cc(CN5CCC6(CC5)CC6)[nH]c4c3=O)c2)(c2nncn2C)C1] |
| 51 | 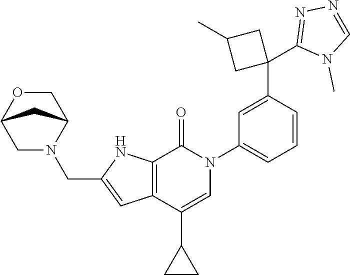 [SMILES: CC1CC(c2cccc(-n3cc(C4CC4)c4cc(CN5C[C@H]6C[C@@H]5CO6)[nH]c4c3=O)c2)(c2nncn2C)C1] | 52 | 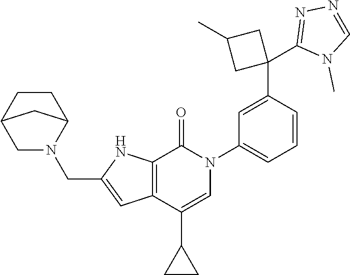 [SMILES: CC1CC(c2cccc(-n3cc(C4CC4)c4cc(CN5CC6CCC5C6)[nH]c4c3=O)c2)(c2nncn2C)C1] |
| 53 | 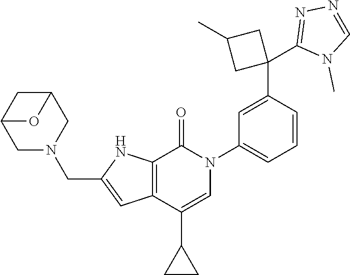 [SMILES: CC1CC(c2cccc(-n3cc(C4CC4)c4cc(CN5CC6CC(C5)O6)[nH]c4c3=O)c2)(c2nncn2C)C1] | 54 | 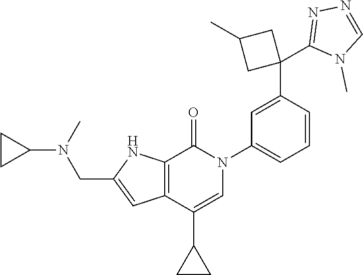 [SMILES: CC1CC(c2cccc(-n3cc(C4CC4)c4cc(CN(C)C5CC5)[nH]c4c3=O)c2)(c2nncn2C)C1] |
| 55 | 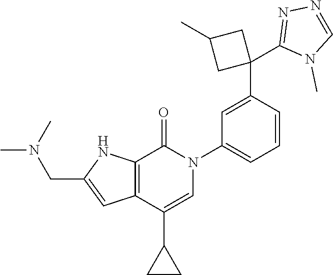 [SMILES: CC1CC(c2cccc(-n3cc(C4CC4)c4cc(CN(C)C)[nH]c4c3=O)c2)(c2nncn2C)C1] | 56 | 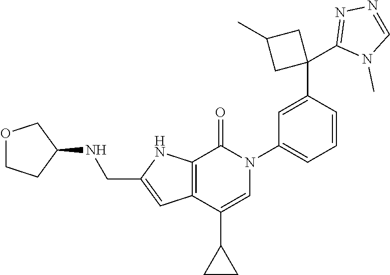 [SMILES: CC1CC(c2cccc(-n3cc(C4CC4)c4cc(CN[C@H]5CCOC5)[nH]c4c3=O)c2)(c2nncn2C)C1] |
| 57 | 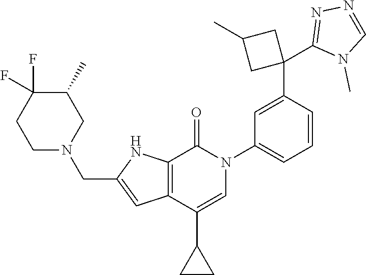 [SMILES: CC1CC(c2cccc(-n3cc(C4CC4)c4cc(CN5CCC(F)(F)[C@H](C)C5)[nH]c4c3=O)c2)(c2nncn2C)C1] | 58 | 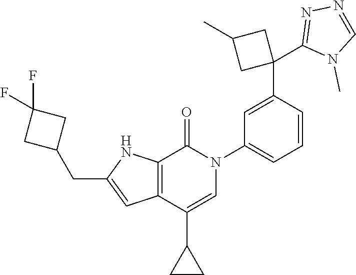 [SMILES: CC1CC(c2cccc(-n3cc(C4CC4)c4cc(CC5CC(F)(F)C5)[nH]c4c3=O)c2)(c2nncn2C)C1] |
| 59 | 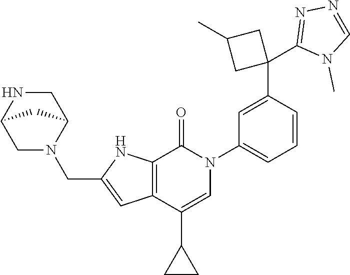 [SMILES: CC1CC(c2cccc(-n3cc(C4CC4)c4cc(CN5C[C@@H]6C[C@H]5CN6)[nH]c4c3=O)c2)(c2nncn2C)C1] | 60 | 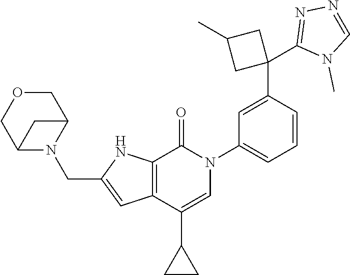 [SMILES: CC1CC(c2cccc(-n3cc(C4CC4)c4cc(CN5C6COCC5C6)[nH]c4c3=O)c2)(c2nncn2C)C1] |
| 61 | 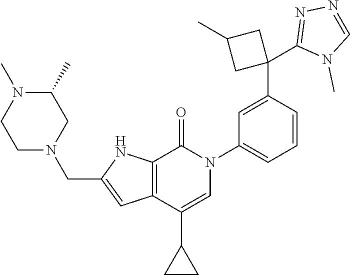 [SMILES: CC1CC(c2cccc(-n3cc(C4CC4)c4cc(CN5CCN(C)[C@H](C)C5)[nH]c4c3=O)c2)(c2nncn2C)C1] | 62 | 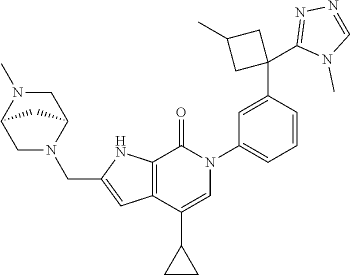 [SMILES: CC1CC(c2cccc(-n3cc(C4CC4)c4cc(CN5C[C@@H]6C[C@H]5CN6C)[nH]c4c3=O)c2)(c2nncn2C)C1] |
| 63 | 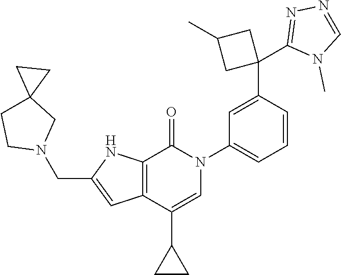 [SMILES: CC1CC(c2cccc(-n3cc(C4CC4)c4cc(CN5CCC6(CC6)C5)[nH]c4c3=O)c2)(c2nncn2C)C1] | 64 | 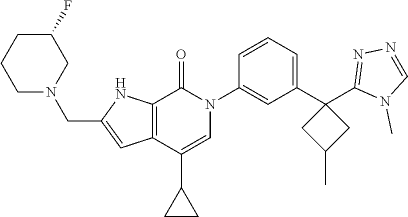 [SMILES: CC1CC(c2cccc(-n3cc(C4CC4)c4cc(CN5CCC[C@H](F)C5)[nH]c4c3=O)c2)(c2nncn2C)C1] |
| 65 | 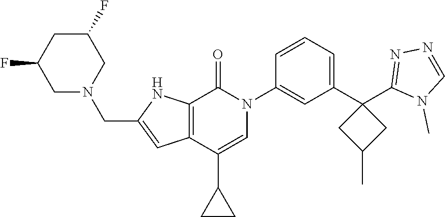 [SMILES: CC1CC(c2cccc(-n3cc(C4CC4)c4cc(CN5C[C@@H](F)C[C@H](F)C5)[nH]c4c3=O)c2)(c2nncn2C)C1] | 66 | 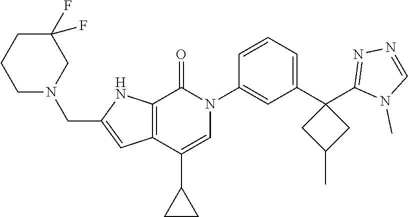 [SMILES: CC1CC(c2cccc(-n3cc(C4CC4)c4cc(CN5CCCC(F)(F)C5)[nH]c4c3=O)c2)(c2nncn2C)C1] |
| 67 | 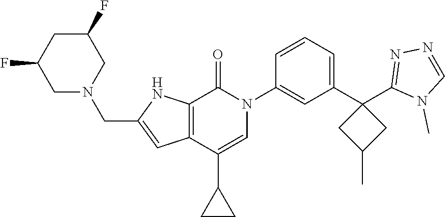 [SMILES: CC1CC(c2cccc(-n3cc(C4CC4)c4cc(CN5C[C@H](F)C[C@H](F)C5)[nH]c4c3=O)c2)(c2nncn2C)C1] | 68 | 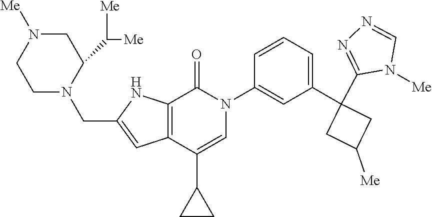 [SMILES: CC1CC(c2cccc(-n3cc(C4CC4)c4cc(CN5CCN(C)C[C@@H]5C(C)C)[nH]c4c3=O)c2)(c2nncn2C)C1] |
| 69 | 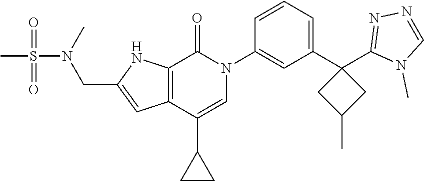 [SMILES: CC1CC(c2cccc(-n3cc(C4CC4)c4cc(CN(C)S(C)(=O)=O)[nH]c4c3=O)c2)(c2nncn2C)C1] | 70 | 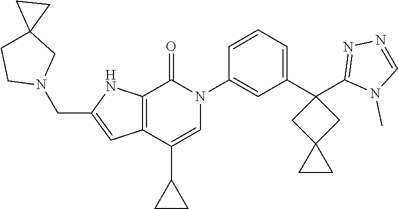 [SMILES: Cn1cnnc1C1(c2cccc(-n3cc(C4CC4)c4cc(CN5CCC6(CC6)C5)[nH]c4c3=O)c2)CC2(CC2)C1] |
| 71 | 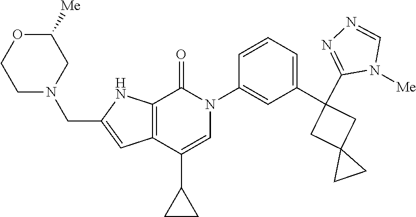 [SMILES: C[C@@H]1CN(Cc2cc3c(C4CC4)cn(-c4cccc(C5(c6nncn6C)CC6(CC6)C5)c4)c(=O)c3[nH]2)CCO1] | 72 | 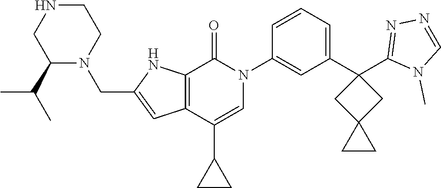 [SMILES: CC(C)[C@H]1CNCCN1Cc1cc2c(C3CC3)cn(-c3cccc(C4(c5nncn5C)CC5(CC5)C4)c3)c(=O)c2[nH]1] |
| 73 | 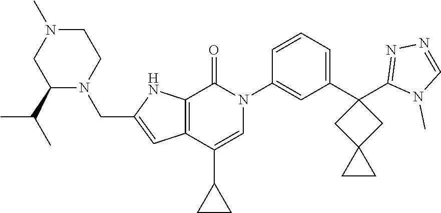 [SMILES: CC(C)[C@H]1CN(C)CCN1Cc1cc2c(C3CC3)cn(-c3cccc(C4(c5nncn5C)CC5(CC5)C4)c3)c(=O)c2[nH]1] | 74 | 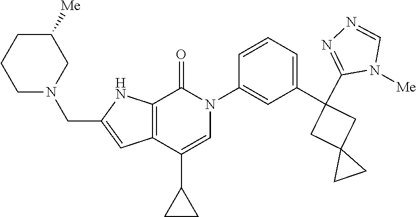 [SMILES: C[C@H]1CCCN(Cc2cc3c(C4CC4)cn(-c4cccc(C5(c6nncn6C)CC6(CC6)C5)c4)c(=O)c3[nH]2)C1] |
| 75 | 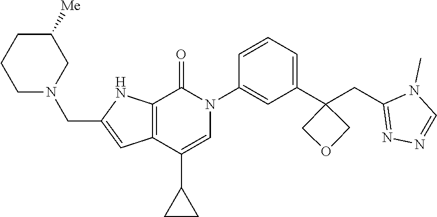 [SMILES: C[C@H]1CCCN(Cc2cc3c(C4CC4)cn(-c4cccc(C5(Cc6nncn6C)COC5)c4)c(=O)c3[nH]2)C1] | 76 | 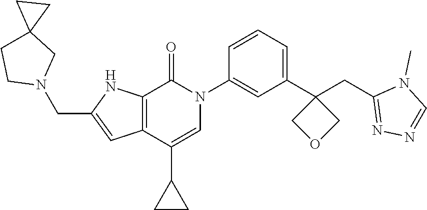 [SMILES: Cn1cnnc1CC1(c2cccc(-n3cc(C4CC4)c4cc(CN5CCC6(CC6)C5)[nH]c4c3=O)c2)COC1] |
| 77 | 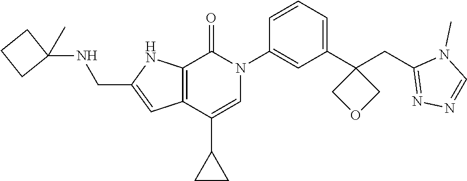 [SMILES: Cn1cnnc1CC1(c2cccc(-n3cc(C4CC4)c4cc(CNC5(C)CCC5)[nH]c4c3=O)c2)COC1] | 78 | 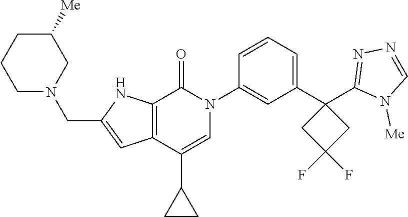 [SMILES: C[C@H]1CCCN(Cc2cc3c(C4CC4)cn(-c4cccc(C5(c6nncn6C)CC(F)(F)C5)c4)c(=O)c3[nH]2)C1] |
| 79 | 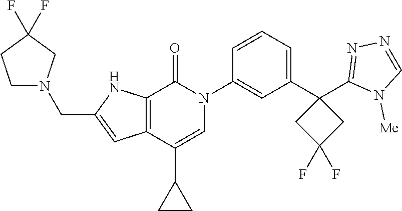 [SMILES: Cn1cnnc1C1(c2cccc(-n3cc(C4CC4)c4cc(CN5CCC(F)(F)C5)[nH]c4c3=O)c2)CC(F)(F)C1] | 80 | 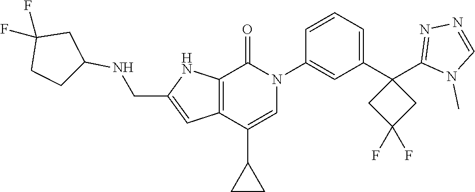 [SMILES: Cn1cnnc1C1(c2cccc(-n3cc(C4CC4)c4cc(CNC5CCC(F)(F)C5)[nH]c4c3=O)c2)CC(F)(F)C1] |
| 81 | 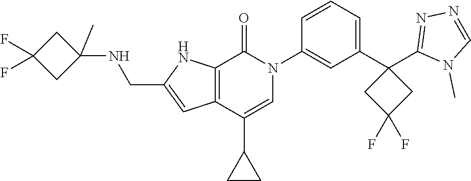 [SMILES: Cn1cnnc1C1(c2cccc(-n3cc(C4CC4)c4cc(CNC5(C)CC(F)(F)C5)[nH]c4c3=O)c2)CC(F)(F)C1] | 82 | 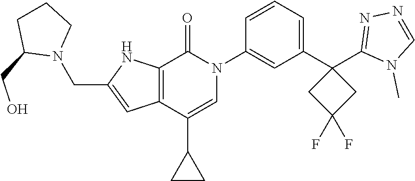 [SMILES: Cn1cnnc1C1(c2cccc(-n3cc(C4CC4)c4cc(CN5CCC[C@@H]5CO)[nH]c4c3=O)c2)CC(F)(F)C1] |
| 83 | 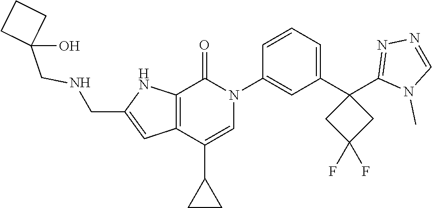 [SMILES: Cn1cnnc1C1(c2cccc(-n3cc(C4CC4)c4cc(CNCC5(O)CCC5)[nH]c4c3=O)c2)CC(F)(F)C1] | 84 | 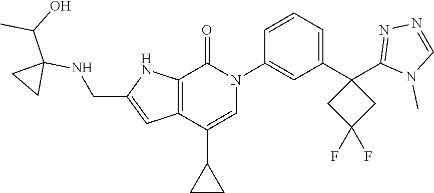 [SMILES: CC(O)C1(NCc2cc3c(C4CC4)cn(-c4cccc(C5(c6nncn6C)CC(F)(F)C5)c4)c(=O)c3[nH]2)CC1] |
| 85 | 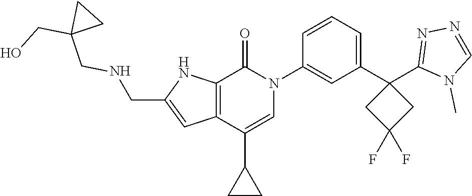 [SMILES: Cn1cnnc1C1(c2cccc(-n3cc(C4CC4)c4cc(CNCC5(CO)CC5)[nH]c4c3=O)c2)CC(F)(F)C1] | 86 | 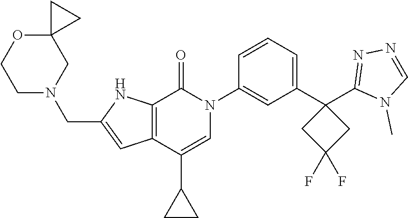 [SMILES: Cn1cnnc1C1(c2cccc(-n3cc(C4CC4)c4cc(CN5CCOC6(CC6)C5)[nH]c4c3=O)c2)CC(F)(F)C1] |
| 87 | 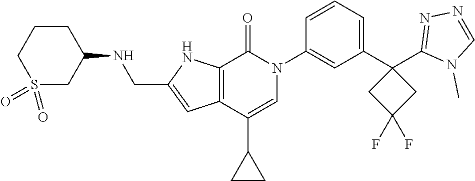 | 88 | 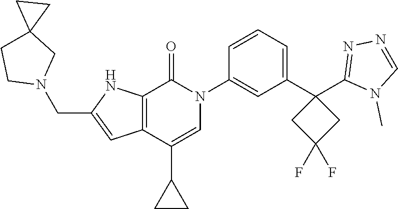 [SMILES: Cn1cnnc1C1(c2cccc(-n3cc(C4CC4)c4cc(CN5CCC6(CC6)C5)[nH]c4c3=O)c2)CC(F)(F)C1] |
| 89 | 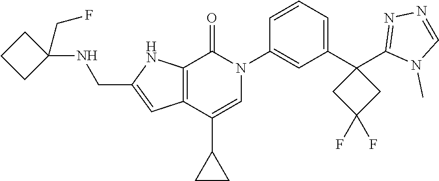 [SMILES: Cn1cnnc1C1(c2cccc(-n3cc(C4CC4)c4cc(CNC5(CF)CCC5)[nH]c4c3=O)c2)CC(F)(F)C1] | 90 | 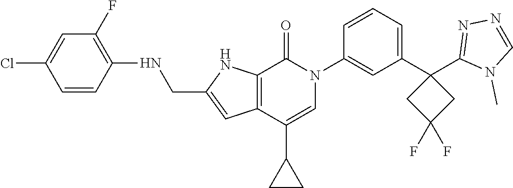 [SMILES: Cn1cnnc1C1(c2cccc(-n3cc(C4CC4)c4cc(CNc5ccc(Cl)cc5F)[nH]c4c3=O)c2)CC(F)(F)C1] |
| 91 | 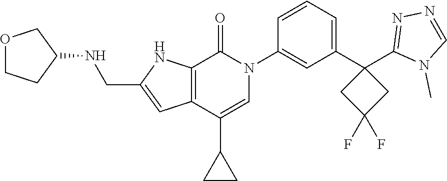 | 92 | 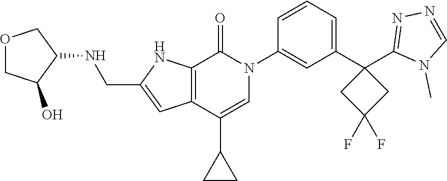 |
| 93 | 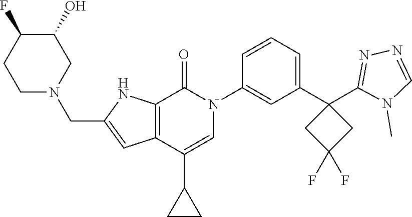 [SMILES: Cn1cnnc1C1(c2cccc(-n3cc(C4CC4)c4cc(CN5CC[C@@H](F)[C@H](O)C5)[nH]c4c3=O)c2)CC(F)(F)C1] | 94 | 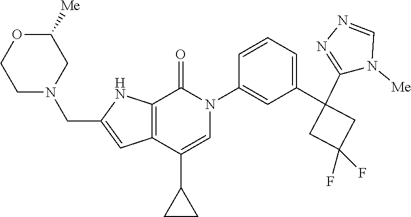 [SMILES: C[C@@H]1CN(Cc2cc3c(C4CC4)cn(-c4cccc(C5(c6nncn6C)CC(F)(F)C5)c4)c(=O)c3[nH]2)CCO1] |
| 95 | 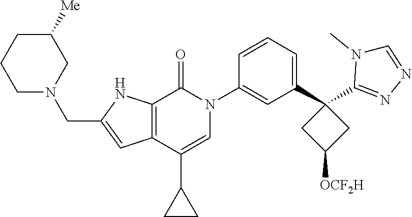 | 96 | 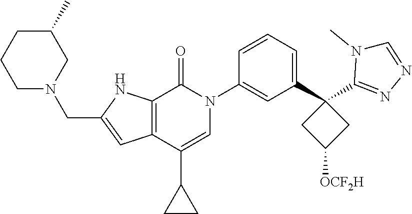 |
| 97 | 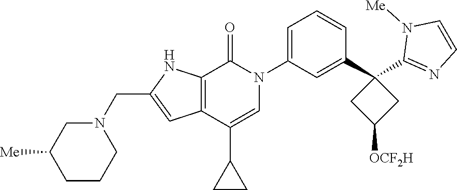 | 98 | 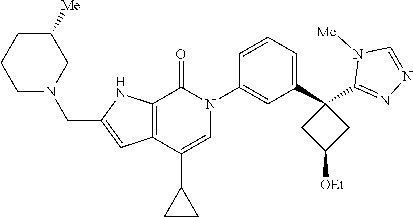 [SMILES: CCO[C@H]1C[C@](c2cccc(-n3cc(C4CC4)c4cc(CN5CCC[C@H](C)C5)[nH]c4c3=O)c2)(c2nncn2C)C1] |
| 99 | 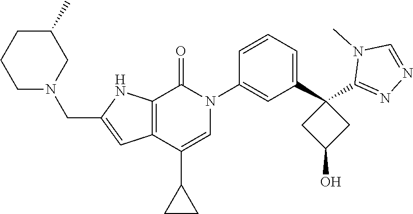 [SMILES: C[C@H]1CCCN(Cc2cc3c(C4CC4)cn(-c4cccc([C@]5(c6nncn6C)C[C@H](O)C5)c4)c(=O)c3[nH]2)C1] | 100 | 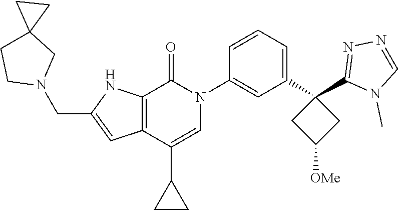 [SMILES: CO[C@H]1C[C@](c2cccc(-n3cc(C4CC4)c4cc(CN5CCC6(CC6)C5)[nH]c4c3=O)c2)(c2nncn2C)C1] |
| 101 | 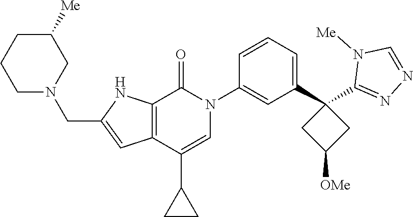 [SMILES: CO[C@H]1C[C@](c2cccc(-n3cc(C4CC4)c4cc(CN5CCC[C@H](C)C5)[nH]c4c3=O)c2)(c2nncn2C)C1] | 102 | 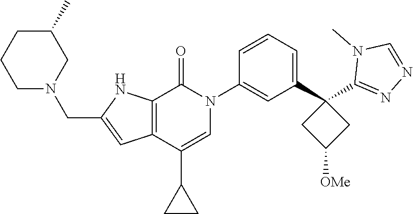 [SMILES: CO[C@H]1C[C@@](c2cccc(-n3cc(C4CC4)c4cc(CN5CCC[C@H](C)C5)[nH]c4c3=O)c2)(c2nncn2C)C1] |
| 103 | 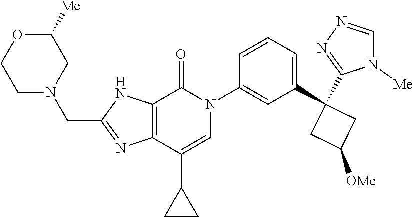 [SMILES: CO[C@H]1C[C@](c2cccc(-n3cc(C4CC4)c4nc(CN5CCO[C@H](C)C5)[nH]c4c3=O)c2)(c2nncn2C)C1] | 104 | 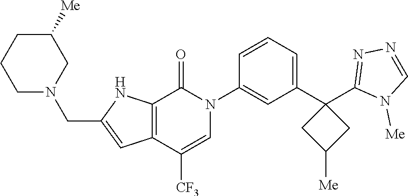 [SMILES: CC1CC(c2cccc(-n3cc(C(F)(F)F)c4cc(CN5CCC[C@H](C)C5)[nH]c4c3=O)c2)(c2nncn2C)C1] |
| 105 | 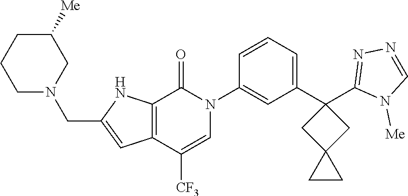 [SMILES: C[C@H]1CCCN(Cc2cc3c(C(F)(F)F)cn(-c4cccc(C5(c6nncn6C)CC6(CC6)C5)c4)c(=O)c3[nH]2)C1] | 106 | 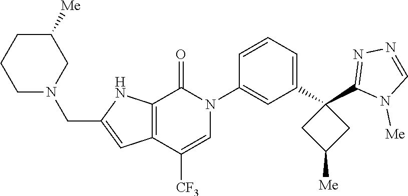 [SMILES: C[C@H]1CCCN(Cc2cc3c(C(F)(F)F)cn(-c4cccc([C@]5(c6nncn6C)C[C@@H](C)C5)c4)c(=O)c3[nH]2)C1] |
| 107 | 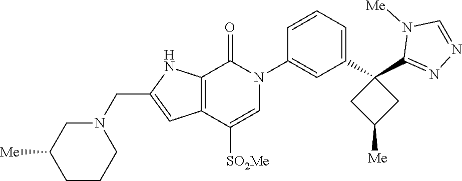 [SMILES: C[C@H]1CCCN(Cc2cc3c(S(C)(=O)=O)cn(-c4cccc([C@]5(c6nncn6C)C[C@@H](C)C5)c4)c(=O)c3[nH]2)C1] | 108 | 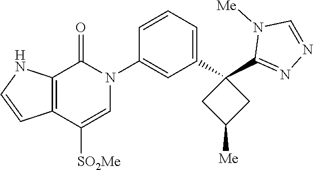 [SMILES: Cn1cnnc1[C@]1(c2cccc(-n3cc(S(C)(=O)=O)c4cc[nH]c4c3=O)c2)C[C@H](C)C1] |
| 109 | 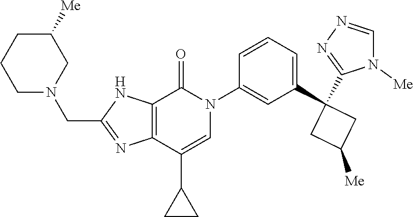 [SMILES: C[C@H]1CCCN(Cc2nc3c(C4CC4)cn(-c4cccc([C@]5(c6nncn6C)C[C@H](C)C5)c4)c(=O)c3[nH]2)C1] | 110 | 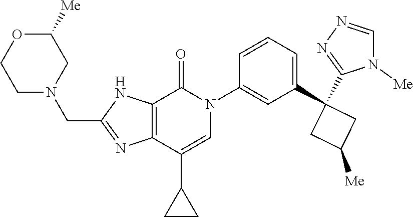 [SMILES: C[C@@H]1CN(Cc2nc3c(C4CC4)cn(-c4cccc([C@]5(c6nncn6C)C[C@H](C)C5)c4)c(=O)c3[nH]2)CCO1] |
| 111 | 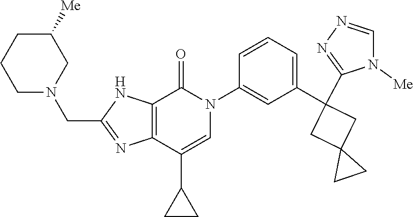 [SMILES: C[C@H]1CCCN(Cc2nc3c(C4CC4)cn(-c4cccc(C5(c6nncn6C)CC6(CC6)C5)c4)c(=O)c3[nH]2)C1] | 112 | 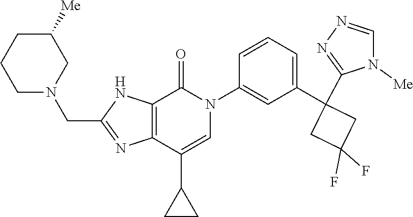 [SMILES: C[C@H]1CCCN(Cc2nc3c(C4CC4)cn(-c4cccc(C5(c6nncn6C)CC(F)(F)C5)c4)c(=O)c3[nH]2)C1] |
| 113 | 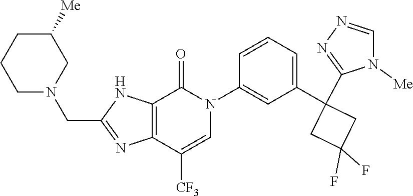 [SMILES: C[C@H]1CCCN(Cc2nc3c(C(F)(F)F)cn(-c4cccc(C5(c6nncn6C)CC(F)(F)C5)c4)c(=O)c3[nH]2)C1] | 114 | 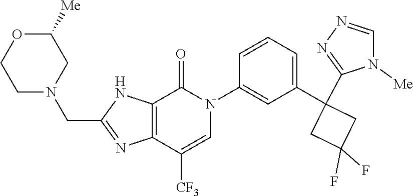 [SMILES: C[C@@H]1CN(Cc2nc3c(C(F)(F)F)cn(-c4cccc(C5(c6nncn6C)CC(F)(F)C5)c4)c(=O)c3[nH]2)CCO1] |
| 115 | 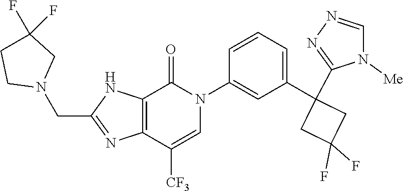 [SMILES: Cn1cnnc1C1(c2cccc(-n3cc(C(F)(F)F)c4nc(CN5CCC(F)(F)C5)[nH]c4c3=O)c2)CC(F)(F)C1] | 116 | 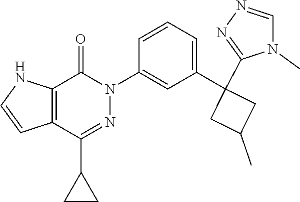 [SMILES: CC1CC(c2cccc(-n3nc(C4CC4)c4cc[nH]c4c3=O)c2)(c2nncn2C)C1] |
| 117 | 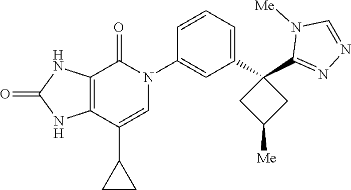 [SMILES: Cn1cnnc1[C@]1(c2cccc(-n3cc(C4CC4)c4[nH]c(=O)[nH]c4c3=O)c2)C[C@H](C)C1] | 118 | 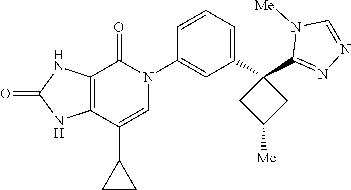 [SMILES: Cn1cnnc1[C@]1(c2cccc(-n3cc(C4CC4)c4[nH]c(=O)[nH]c4c3=O)c2)C[C@@H](C)C1] |
| 119 | 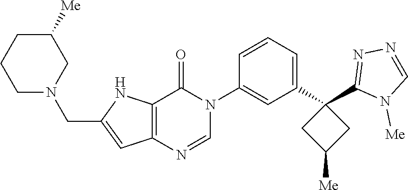 [SMILES: C[C@H]1CCCN(Cc2cc3ncn(-c4cccc([C@]5(c6nncn6C)C[C@@H](C)C5)c4)c(=O)c3[nH]2)C1] | 120 | 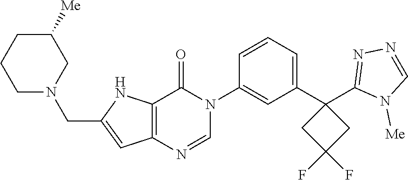 [SMILES: C[C@H]1CCCN(Cc2cc3ncn(-c4cccc(C5(c6nncn6C)CC(F)(F)C5)c4)c(=O)c3[nH]2)C1] |
| 121 | 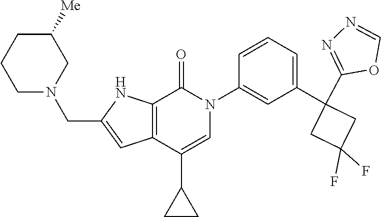 [SMILES: C[C@H]1CCCN(Cc2cc3c(C4CC4)cn(-c4cccc(C5(c6nnco6)CC(F)(F)C5)c4)c(=O)c3[nH]2)C1] | 122/123 | 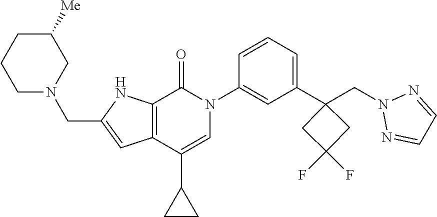 [SMILES: C[C@H]1CCCN(Cc2cc3c(C4CC4)cn(-c4cccc(C5(Cn6nccn6)CC(F)(F)C5)c4)c(=O)c3[nH]2)C1] |
| 122/123 | 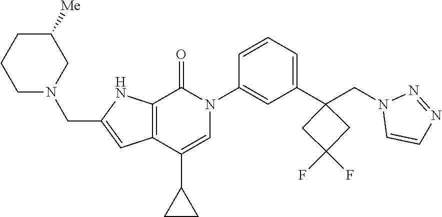 [SMILES: C[C@H]1CCCN(Cc2cc3c(C4CC4)cn(-c4cccc(C5(Cn6ccnn6)CC(F)(F)C5)c4)c(=O)c3[nH]2)C1] | 124 | 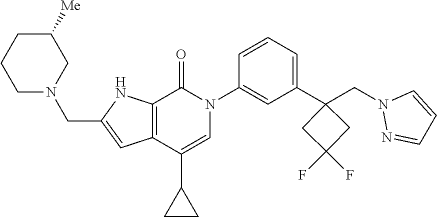 [SMILES: C[C@H]1CCCN(Cc2cc3c(C4CC4)cn(-c4cccc(C5(Cn6cccn6)CC(F)(F)C5)c4)c(=O)c3[nH]2)C1] |
| 125 | 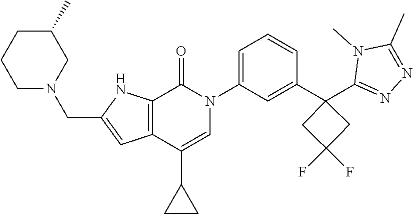 [SMILES: Cc1nnc(C2(c3cccc(-n4cc(C5CC5)c5cc(CN6CCC[C@H](C)C6)[nH]c5c4=O)c3)CC(F)(F)C2)n1C] | 126 | 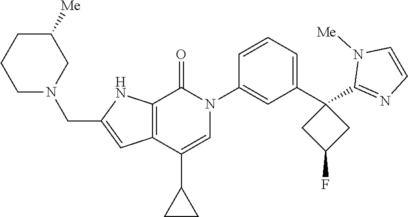 [SMILES: C[C@H]1CCCN(Cc2cc3c(C4CC4)cn(-c4cccc([C@]5(c6nccn6C)C[C@H](F)C5)c4)c(=O)c3[nH]2)C1] |
| 127 | 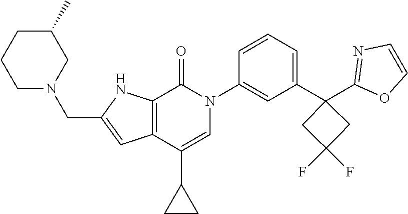 [SMILES: C[C@H]1CCCN(Cc2cc3c(C4CC4)cn(-c4cccc(C5(c6ncco6)CC(F)(F)C5)c4)c(=O)c3[nH]2)C1] | 128 | 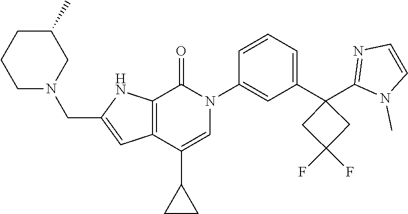 [SMILES: C[C@H]1CCCN(Cc2cc3c(C4CC4)cn(-c4cccc(C5(c6nccn6C)CC(F)(F)C5)c4)c(=O)c3[nH]2)C1] |
| 129 | 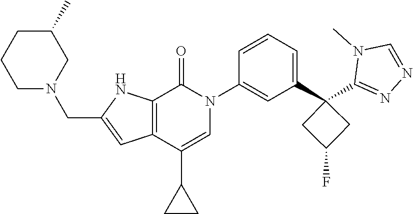 [SMILES: C[C@H]1CCCN(Cc2cc3c(C4CC4)cn(-c4cccc([C@]5(c6nncn6C)C[C@@H](F)C5)c4)c(=O)c3[nH]2)C1] | 130 | 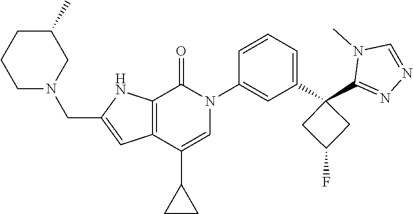 [SMILES: C[C@H]1CCCN(Cc2cc3c(C4CC4)cn(-c4cccc([C@]5(c6nncn6C)C[C@H](F)C5)c4)c(=O)c3[nH]2)C1] |
| 131 | 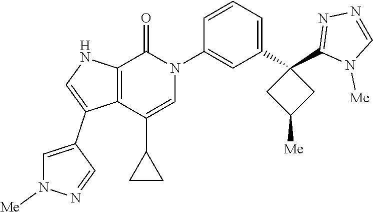 [SMILES: Cn1cc(-c2c[nH]c3c(=O)n(-c4cccc([C@]5(c6nncn6C)C[C@@H](C)C5)c4)cc(C4CC4)c23)cn1] | 132 | 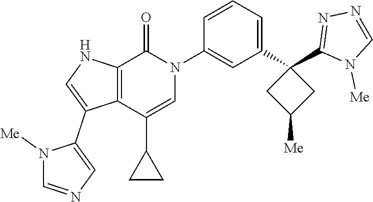 [SMILES: Cn1cncc1-c1c[nH]c2c(=O)n(-c3cccc([C@]4(c5nncn5C)C[C@@H](C)C4)c3)cc(C3CC3)c12] |
| 133 | 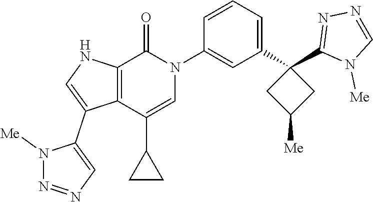 [SMILES: Cn1nncc1-c1c[nH]c2c(=O)n(-c3cccc([C@]4(c5nncn5C)C[C@@H](C)C4)c3)cc(C3CC3)c12] | 134 | 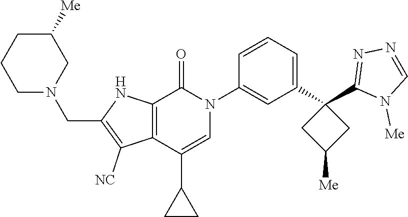 [SMILES: C[C@H]1CCCN(Cc2[nH]c3c(=O)n(-c4cccc([C@]5(c6nncn6C)C[C@@H](C)C5)c4)cc(C4CC4)c3c2C#N)C1] |
| 135 | 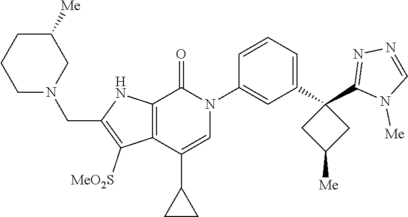 [SMILES: C[C@H]1CCCN(Cc2[nH]c3c(=O)n(-c4cccc([C@]5(c6nncn6C)C[C@@H](C)C5)c4)cc(C4CC4)c3c2S(C)(=O)=O)C1] | 136 | 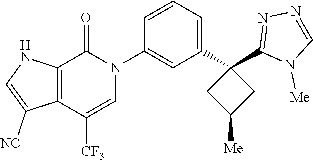 [SMILES: Cn1cnnc1[C@]1(c2cccc(-n3cc(C(F)(F)F)c4c(C#N)c[nH]c4c3=O)c2)C[C@H](C)C1] |
| 137 | 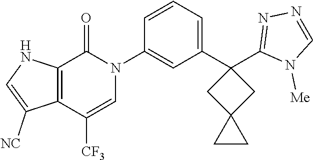 [SMILES: Cn1cnnc1C1(c2cccc(-n3cc(C(F)(F)F)c4c(C#N)c[nH]c4c3=O)c2)CC2(CC2)C1] | 138 | 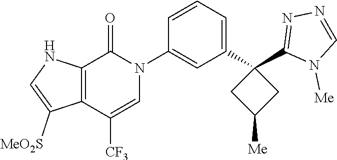 [SMILES: Cn1cnnc1[C@]1(c2cccc(-n3cc(C(F)(F)F)c4c(S(C)(=O)=O)c[nH]c4c3=O)c2)C[C@H](C)C1] |
| 139 | 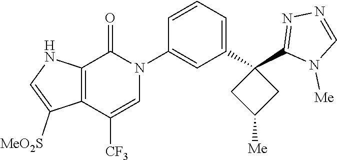 [SMILES: Cn1cnnc1[C@]1(c2cccc(-n3cc(C(F)(F)F)c4c(S(C)(=O)=O)c[nH]c4c3=O)c2)C[C@@H](C)C1] | 140 | 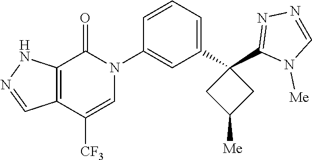 [SMILES: Cn1cnnc1[C@]1(c2cccc(-n3cc(C(F)(F)F)c4cn[nH]c4c3=O)c2)C[C@H](C)C1] |
| 141 | 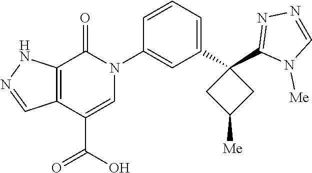 [SMILES: Cn1cnnc1[C@]1(c2cccc(-n3cc(C(=O)O)c4cn[nH]c4c3=O)c2)C[C@H](C)C1] | 142 | 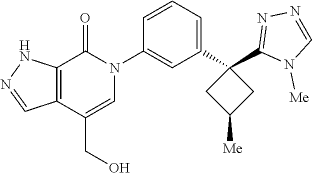 [SMILES: Cn1cnnc1[C@]1(c2cccc(-n3cc(CO)c4cn[nH]c4c3=O)c2)C[C@H](C)C1] |
| 143 | 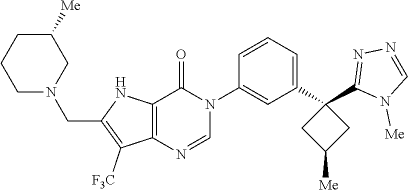 [SMILES: C[C@H]1CCCN(Cc2[nH]c3c(=O)n(-c4cccc([C@]5(c6nncn6C)C[C@@H](C)C5)c4)cnc3c2C(F)(F)F)C1] | 144 | 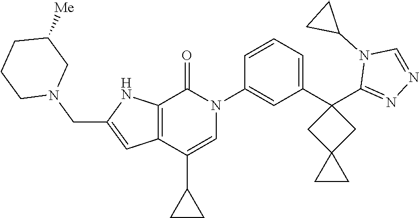 [SMILES: C[C@H]1CCCN(Cc2cc3c(C4CC4)cn(-c4cccc(C5(c6nncn6C6CC6)CC6(CC6)C5)c4)c(=O)c3[nH]2)C1] |
| 145 | 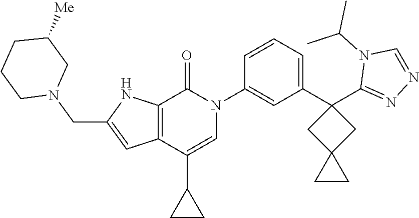 [SMILES: CC(C)n1cnnc1C1(c2cccc(-n3cc(C4CC4)c4cc(CN5CCC[C@H](C)C5)[nH]c4c3=O)c2)CC2(CC2)C1] | 146 | 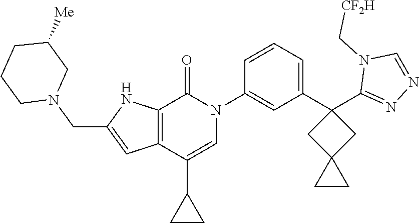 [SMILES: C[C@H]1CCCN(Cc2cc3c(C4CC4)cn(-c4cccc(C5(c6nncn6CC(F)F)CC6(CC6)C5)c4)c(=O)c3[nH]2)C1] |
| 147 | 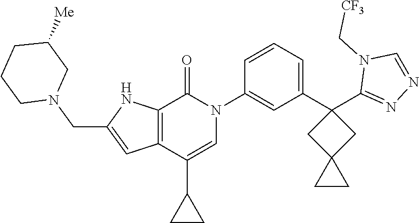 [SMILES: C[C@H]1CCCN(Cc2cc3c(C4CC4)cn(-c4cccc(C5(c6nncn6CC(F)(F)F)CC6(CC6)C5)c4)c(=O)c3[nH]2)C1] | 148 | 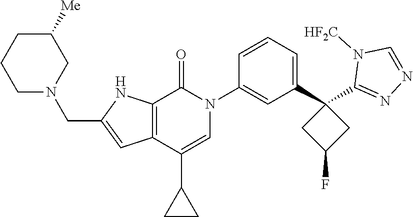 |
| 149 | 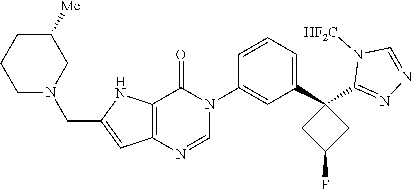 | 150 | 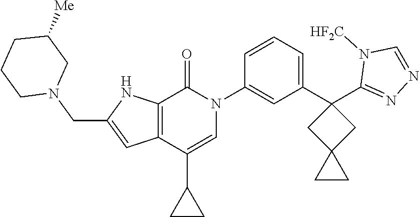 |
| 151 | 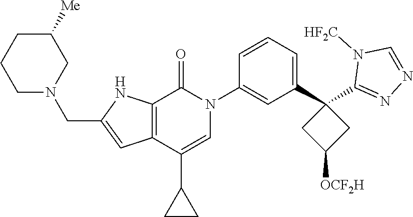 | 152 | 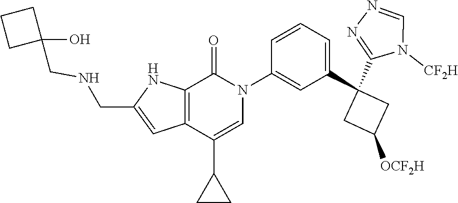 |
| 153 | 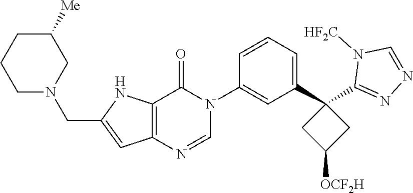 | 154 | 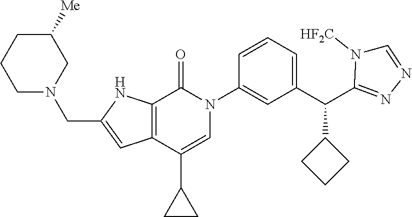 |
| 155 | 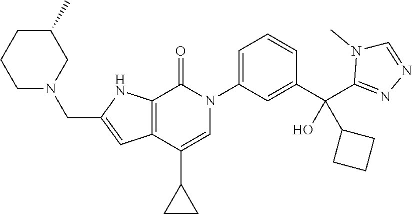 [SMILES: C[C@H]1CCCN(Cc2cc3c(C4CC4)cn(-c4cccc(C(O)(c5nncn5C)C5CCC5)c4)c(=O)c3[nH]2)C1] | 156 | 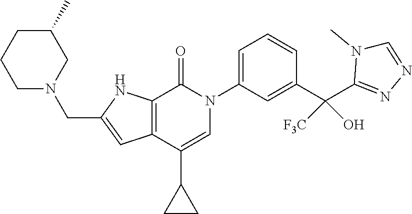 [SMILES: C[C@H]1CCCN(Cc2cc3c(C4CC4)cn(-c4cccc(C(O)(c5nncn5C)C(F)(F)F)c4)c(=O)c3[nH]2)C1] |
| 157 | 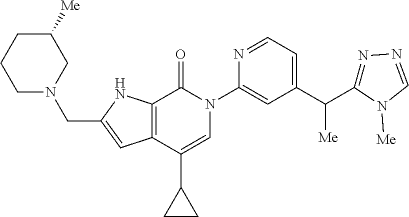 [SMILES: CC(c1ccnc(-n2cc(C3CC3)c3cc(CN4CCC[C@H](C)C4)[nH]c3c2=O)c1)c1nncn1C] | 158 | 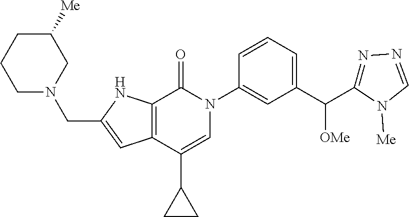 [SMILES: COC(c1cccc(-n2cc(C3CC3)c3cc(CN4CCC[C@H](C)C4)[nH]c3c2=O)c1)c1nncn1C] |
| 159 | 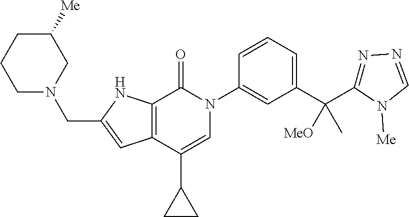 [SMILES: COC(C)(c1cccc(-n2cc(C3CC3)c3cc(CN4CCC[C@H](C)C4)[nH]c3c2=O)c1)c1nncn1C] | 160 | 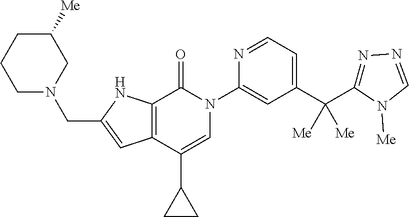 [SMILES: C[C@H]1CCCN(Cc2cc3c(C4CC4)cn(-c4cc(C(C)(C)c5nncn5C)ccn4)c(=O)c3[nH]2)C1] |
| 161 | 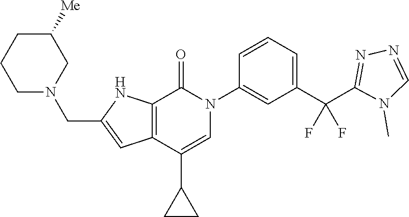 [SMILES: C[C@H]1CCCN(Cc2cc3c(C4CC4)cn(-c4cccc(C(F)(F)c5nncn5C)c4)c(=O)c3[nH]2)C1] | 162 | 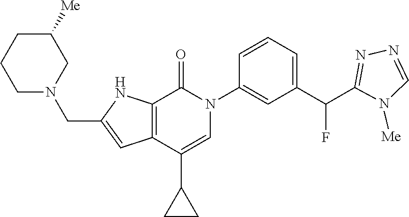 [SMILES: C[C@H]1CCCN(Cc2cc3c(C4CC4)cn(-c4cccc(C(F)c5nncn5C)c4)c(=O)c3[nH]2)C1] |
| 163 | 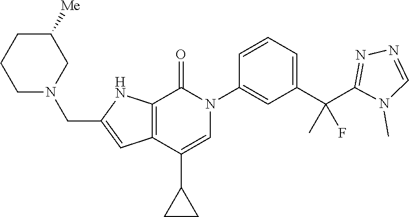 [SMILES: C[C@H]1CCCN(Cc2cc3c(C4CC4)cn(-c4cccc(C(C)(F)c5nncn5C)c4)c(=O)c3[nH]2)C1] | 164 | 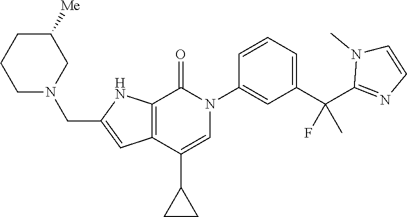 [SMILES: C[C@H]1CCCN(Cc2cc3c(C4CC4)cn(-c4cccc(C(C)(F)c5nccn5C)c4)c(=O)c3[nH]2)C1] |
| 165 | 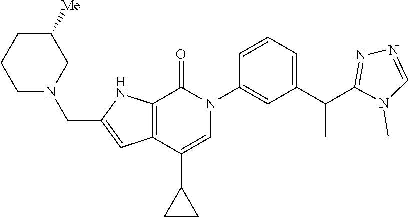 [SMILES: CC(c1cccc(-n2cc(C3CC3)c3cc(CN4CCC[C@H](C)C4)[nH]c3c2=O)c1)c1nncn1C] | | |

### Therapeutic and Prophylactic Uses

The present disclosure provides methods for using compounds described herein in the preparation of a medicament for inhibiting Cbl-b. As used herein, the terms “inhibit”, “inhibition” and the like refer to the ability of a compound to decrease the function or activity of a particular target, e.g., Cbl-b. The decrease is preferably at least 50% and may be, for example, at least about 55%, at least about 60%, at least about 65%, at least about 70%, at least about 75%, at least about 80%, at least about 85%, at least about 90%, or at least about 95%. The present disclosure also encompasses the use of the compounds described herein in the preparation of a medicament for the treatment or prevention of diseases, disorders, and/or conditions that would benefit from inhibition of Cbl-b. As one example, the present disclosure encompasses the use of the compounds described herein in the preparation of a medicament for the treatment of cancer. In another example, the present disclosure encompasses the use of the compounds described herein in the preparation of a medicament for the treatment of an infectious disease, optionally a viral infection. In some embodiments of the aforementioned methods, the compounds described herein are used in combination with at least one additional therapy, examples of which are set forth elsewhere herein.

Cbl-b is an E3 ubiquitin ligase that acts by ubiquitinating proteins leading to their degradation or altered subcellular localization. More specifically, Cbl-b acts by binding ubiquitin-conjugating enzyme (E2) loaded with ubiquitin and substrate to facilitate formation of an isopeptide bond between the C-terminal carboxyl of ubiquitin and the F-amino group of a substrate lysine side chain or free N-terminal amino group. Through this activity, Cbl-b functions, in one aspect, as a negative regulator of immune cell activation. For example, Cbl-b inhibits T cell activation through ubiquitination of intracellular signaling proteins, including but not limited to pTYR-containing proteins (e.g., ZAP-70, etc.), p85 regulatory subunit of phosphatidynlinositol 3 kinase (PI3K), PLCγ1, and PKC6. Cbl-b is also believed to negatively regulate cytokine-induced or target-induced NK cell cytotoxicity and cytokine production. Cbl-b has also been implicated in immunosuppressive signaling pathways, such as PD-1, CTLA-4, CD155, and TGF-β.

As demonstrated herein, the use of compounds described herein potently inhibits Cbl-b activity, resulting in increased immune cell activity. Diseases, disorders, and/or conditions that would benefit from Cbl-b inhibition may include those where greater immune cell (e.g., T cell, NK cell, etc.) activation is desired and/or there is limited immune cell stimulation, for example, due to low antigen density, poor quality neoantigen, high PD-L1 expression, or combinations thereof.

Accordingly, in some embodiments, the compounds described herein are administered to a subject in need thereof in an amount effective to inhibit Cbl-b activity. In one example, a measure of Cbl-b inhibition may be decreased ubiquitination of intracellular signaling proteins targeted by Cbl-b. Non-limiting examples of intracellular signaling proteins targeted by Cbl-b include pTYR-containing proteins (e.g., ZAP-70, etc.), p85 regulatory subunit of phosphatidynlinositol 3 kinase (PI3K), PLCγ1, and PKC6. Cbl-b activity may be assessed using primary immune cells (e.g., T cells, NK cells) obtained from a peripheral blood sample or a tissue sample (e.g., a tumor sample) that was obtained from the subject. Activity may be determined, for example, by comparison to a previous sample obtained from the subject (i.e., prior to administration of the compound) or by comparison to a reference value for a control group (e.g., standard of care, a placebo, etc.).

Alternatively or in addition, in some embodiments, the compounds described herein are administered to a subject in need thereof in an amount effective to increase immune cell expansion, proliferation, activation and/or activity, as compared to a suitable control (e.g., a subject receiving standard of care, a subject receiving not treatment or a placebo treatment, etc.). Immune cell expansion, proliferation, activation and activity may be assessed using cells obtained from a peripheral blood sample or a tissue sample (e.g., a tumor sample) that was obtained from the subject. Immune cell numbers in tissue or blood may be quantified (absolute numbers or relative numbers) by immunophenotyping, i.e., a process of using antibodies (or other antigen-specific reagent) to detect and quantify cell-associated antigens. Lymphoid cell markers may include but are not limited to CD3, CD4, CD8, CD16, CD25, CD39, CD45, CD56, CD103, CD127, and FOXP3. CD4 and CD8 can distinguish T cell with different effector functions (e.g., CD4+ T cells and CD8+ T cells). Co-expression of different cell markers can further distinguish sub-groups. For example, co-expression of CD39 and CD103 can differentiate tumor-specific T cells (CD8+CD39+CD103+ T cells) from bystander T cells in the tumor microenvironment (TME). For myeloid cells, suitable markers may include but are not limited to CD14, CD68, CD80, CD83, CD86, CD163, and CD206. Ki67 is a non-limiting example of a suitable marker of cell proliferation, such that an increase in Ki67 positive cells (e.g., CD8+ T cells, NK cells, etc.) as compared to a reference sample indicate cell proliferation. The term “activation” refers to the state of an immune cell that has been sufficiently primed to induce detectable effector functions (i.e., immune cell activity) upon stimulation. For example, T cells may be stimulated through the TCR/CD3 complex alone or with one or more secondary costimulatory signals. Non-limiting examples of measures of increased immune cell activity (i.e. effector function) may include increased expression, production and/or secretion of chemokines, pro-inflammatory cytokines and/or cytotoxic factors, increased cytotoxic activity, and increased gene expression and/or cell surface markers related to immune cell function and immune signaling. Examples of pro-inflammatory cytokines include, but are not limited to, IL-1a, IL-1b, IL-2, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of cytotoxic factors include, but are not limited to, granzyme A, granzyme B, soluble Fas ligand (sFasL), and perforin.

In some embodiments, the compounds described herein are administered to a subject in need thereof in an amount effective to increase T cell expansion, proliferation, activity, or any combination thereof. In certain embodiments, the T cells are CD8+ T cells, optionally tumor infiltrating CD8+ T cells and/or antigen experienced CD8+ T cells. In some embodiments, the T cells are CD8+CD39+CD103+ T cells. In embodiments directed to increased T cell activation and/or activity, measures of increased T cell activity may be increased T cell expression, production or secretion of chemokines, pro-inflammatory cytokines (e.g., IFNγ, TNF-α, IL-2, etc.) and/or cytotoxic factors (e.g. perforin, Granzyme B, etc.); increased pro-inflammatory cytokine levels in the tumor microenvironment; increased T cell receptor (TCR) signaling; increased glucose uptake; increased glycolysis; and increased killing of cancer cells. In some embodiments, the compounds described herein are administered to a subject in need thereof in an amount effective to increase activity, optionally wherein a measure of T cell activity is production and/or secretion of one or more pro-inflammatory cytokine, optionally wherein one or more pro-inflammatory cytokine is IFNγ, TNF-α, or IL-2.

In some embodiments, the compounds described herein are administered to a subject in need thereof in an amount effective to increase NK cell expansion, proliferation, activity, or any combination thereof. In embodiments directed to increased NK cell activity, measures of increased NK cell activity may be increased NK cell expression, production or secretion of chemokines, inflammatory cytokines (e.g., IFNγ, TNF-α, IL-2, etc.) and/or cytotoxic factors (e.g. perform, Granzyme B, etc.); increased inflammatory cytokine levels in the tumor microenvironment; and increased killing of cancer cells.

Alternatively or in addition, in some embodiments, the compounds described herein are administered to a subject in need thereof to treat and/or prevent cancer or a cancer-related disease, disorder or condition. In some embodiments, the compounds described herein are administered to a subject in need thereof to treat cancer, optionally in combination with at least one additional therapy, examples of which are set forth elsewhere herein.

Alternatively or in addition, in some embodiments, the compounds described herein are administered to a subject in need thereof to treat and/or prevent an infection. In some embodiments, the compounds described herein are administered to a subject in need thereof to treat and/or prevent a viral infection. In some embodiments, the viral infection is a disease caused by hepatitis C virus (HCV), human papilloma virus (HPV), cytomegalovirus (CMV), herpes simplex virus (HSV), Epstin-Barr virus (EBV), varicella zoster virus, coxsackie virus, human immunodeficiency virus (HIV), or lymphocytic choriomeningitis virus (LCMV).

Alternatively or in addition, in some embodiments, the compounds described herein are brought into contact with an immune cell or a plurality of immune cells, in vitro or ex vivo, in an amount effective to increase proliferation, activation or activity of the immune cell(s). In some embodiments, the immune cell(s) may be allogenic immune cell(s) collected from one or more subject. In some embodiments, the immune cell(s) may be autologous immune cell(s) collected from a subject in need of treatment. In certain embodiments, the cells may be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In various embodiments, the immune cells may be genetically modified to target the cells to a specific antigen and/or enhance the cells' anti-tumor effects (e.g., engineered T cell receptor (TCR) cellular therapies, chimeric antigen receptor (CAR) cellular therapies, etc.). In some embodiments, the immune cell(s) are then administered to a subject in need thereof to treat and/or prevent cancer or a cancer-related disease, disorder or condition. In some embodiments, the immune cells are administered to a subject in need thereof to treat cancer, optionally in combination with at least one additional therapy, examples of which are set forth elsewhere herein.

In one or more embodiments, the compounds described herein are useful in the treatment and/or prophylaxis of cancer (e.g., carcinomas, sarcomas, leukemias, lymphomas, myelomas, etc.). In certain embodiments, the cancer may be locally advanced and/or unresectable, metastatic, or at risk of becoming metastatic. Alternatively, or in addition, the cancer may be recurrent or no longer responding to a treatment, such as a standard of care treatment known to one of skill in the art. Exemplary types of cancer contemplated by this disclosure include cancer of the genitourinary tract (e.g., bladder, kidney, renal cell, penile, prostate, testicular, Von Hippel-Lindau disease, etc.), uterus, cervix, ovary, breast, gastrointestinal tract (e.g., esophagus, oropharynx, stomach, small or large intestines, colon, or rectum), bone, bone marrow, skin (e.g., melanoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), and the immune system (e.g., spleen or thymus).

In some embodiments, the compounds according to this disclosure are useful in the treatment and/or prophylaxis of hematological malignancies. Exemplary types of cancer affecting the hematopoietic system include leukemias, lymphomas and myelomas, including acute myeloid leukemia, adult T-cell leukemia, T-cell large granular lymphocyte leukemia, acute lymphoblastic leukemia, chronic lymphocytic leukemia, chronic myelogenous leukemia, acute monocytic leukemia, Hodgkin's and Non-Hodgkin's lymphoma, Diffuse large B Cell lymphoma, and multiple myeloma. In a specific embodiment, the compounds according to this disclosure are useful in the treatment of Diffuse large B Cell lymphoma, optionally Diffuse large B Cell lymphoma with Richter transformation.

In another embodiment, the compounds according to this disclosure are useful in the treatment and/or prophylaxis of solid tumors. The solid tumor may be, for example, ovarian cancer, endometrial cancer, breast cancer, lung cancer (small cell or non-small cell), colon cancer, prostate cancer, cervical cancer, biliary cancer, pancreatic cancer, gastric cancer, esophageal cancer, liver cancer (hepatocellular carcinoma), kidney cancer (renal cell carcinoma), head-and-neck tumors, mesothelioma, melanoma, sarcomas, central nervous system (CNS) hemangioblastomas, and brain tumors (e.g., gliomas, such as astrocytoma, oligodendroglioma and glioblastomas).

In another embodiment, the compounds according to this disclosure are useful in the treatment and/or prophylaxis of breast cancer, genitourinary cancer, gastrointestinal cancer, lung cancer, skin cancer, or a combination thereof.

In some embodiments, the compounds according to this disclosure are useful in the treatment of breast cancer. In further embodiments, the breast cancer is hormone receptor positive (e.g., ERa-positive breast cancer, PR-positive breast cancer, ERa-positive and PR-positive breast cancer), HER2 positive breast cancer, HER2 over-expressing breast cancer, or any combination thereof. In still further embodiments, the breast cancer is triple negative breast cancer.

In some embodiments, the compounds according to this disclosure are useful in the treatment of genitourinary cancer. In further embodiments, the genitourinary cancer is gynecologic cancer. In still further embodiments, the gynecologic cancer is cervical cancer, ovarian cancer, vaginal cancer, vulvar cancer, endometrial cancer, peritoneal cancer, or fallopian tube carcinoma.

In still further embodiments, the genitourinary cancer is urothelial cancer. In still further embodiments, the genitourinary cancer is prostate cancer, optionally castration-resistant prostate cancer. In further embodiments, the genitourinary cancer is bladder cancer. In still further embodiments, the genitourinary cancer is peritoneal cancer, optionally primary peritoneal cancer.

In some embodiments, the compounds according to this disclosure are useful in the treatment of head and neck cancer. In further embodiments, the head and neck cancer is head and neck squamous cell carcinoma.

In some embodiments, the compounds according to this disclosure are useful in the treatment of skin cancer. In further embodiments, the skin cancer is melanoma.

In some embodiments, the compounds according to this disclosure are useful in the treatment of lung cancer. In further embodiments, the lung cancer is mesothelioma or non-small cell lung cancer (NSCLC). In still further embodiments, the NSCLC is lung squamous cell carcinoma or lung adenocarcinoma.

In some embodiments, the compounds according to this disclosure are useful in the treatment of gastrointestinal cancer. In some embodiments, the gastrointestinal cancer is upper GI cancer, such as esophageal or gastric cancer. In further embodiments, the upper GI cancer is an adenocarcinoma, a squamous cell carcinoma, or any combination thereof. In still further embodiments, the upper GI cancer is esophageal adenocarcinoma (EAC), esophageal squamous cell carcinoma (ESCC), gastroesophageal junction adenocarcinoma (GEJ), gastric adenocarcinoma (also referred to herein as “gastric cancer”) or any combination thereof. In some embodiments, the gastrointestinal cancer is lower GI cancer. In further embodiments, the lower GI cancer is colorectal cancer.

In some embodiments, the compounds according to this disclosure are useful in the treatment of a neuroendocrine tumor. In further embodiments, the neuroendocrine tumor is pancreatic neuroendocrine tumor, pheochromocytoma, paraganglioma, or a tumor of the adrenal gland.

In some embodiments, the compounds according to this disclosure are useful in the treatment of brain cancer. In further embodiments, the brain cancer is a glioma. In still further embodiments, the glioma is an astrocytoma, an oligodendroglioma, or a glioblastoma.

In some embodiments, the compounds according to this disclosure are useful in the treatment of kidney cancer. In further embodiments, the kidney cancer is renal cell carcinoma. In still further embodiments, the renal cell carcinoma is clear cell renal carcinoma.

In some embodiments, the compounds according to this disclosure are useful in the treatment of pancreatic cancer. In further embodiments, the pancreatic cancer is pancreatic neuroendocrine tumor or pancreatic adenocarcinoma.

In the aforementioned embodiments, the methods of the present disclosure may be practiced in an adjuvant setting or neoadjuvant setting, optionally in the treatment of locally advanced, unresectable, or metastatic cancer. Alternatively or in addition, the methods described herein may be indicated as a first line, second line, third line, or greater line of treatment, optionally in the treatment of locally advanced, unresectable, or metastatic cancer.

The present disclosure also provides methods of treating or preventing other cancer-related diseases, disorders or conditions. The use of the term(s) cancer-related diseases, disorders and conditions is meant to refer broadly to conditions that are associated, directly or indirectly, with cancer and non-cancerous proliferative disease, and includes, e.g., angiogenesis, precancerous conditions such as dysplasia, and non-cancerous proliferative diseases disorders or conditions, such as benign proliferative breast disease and papillomas. For clarity, the term(s) cancer-related disease, disorder and condition do not include cancer per se.

In general, the disclosed methods for treating or preventing cancer, or a cancer-related disease, disorder or condition, in a subject in need thereof comprise administering to the subject a compound disclosed herein, or a pharmaceutically acceptable salt thereof. In some embodiments, the present disclosure provides methods for treating or preventing cancer, or a cancer-related disease, disorder or condition with a compound disclosed herein, or a pharmaceutically acceptable salt thereof, and at least one additional therapy, examples of which are set forth elsewhere herein.

In particular embodiments of the present disclosure, the compounds are used to increase or enhance an immune response to an antigen by providing adjuvant activity. In a particular embodiment, at least one antigen or vaccine is administered to a subject in combination with at least one compound of the present disclosure to prolong an immune response to the antigen or vaccine. Therapeutic compositions are also provided which include at least one antigenic agent or vaccine component, including, but not limited to, viruses, bacteria, and fungi, or portions thereof, proteins, peptides, tumor-specific antigens, and nucleic acid vaccines, in combination with at least one compound of the present disclosure.

In some instances, the methods according to this disclosure may be provided in selected patients, for example subjects identified as having in a relevant tissue or sample, e.g., detectable PD-L1 expression, high microsatellite instability, high tumor mutational burden, or any combination thereof. In some instances, the subject is identified as having an oncogene driven cancer that has a mutation in at least one gene associated with the cancer.

In some embodiments, patients are selected by assessing the expression of relevant biomarkers, e.g., PD-L1 expression, microsatellite instability markers, etc., in a relevant sample, such as a peripheral blood sample or a tumor biopsy, using immunohistochemistry, immunophenotyping, PCR-based amplification, RNA sequencing, or other clinically validated assay. In one embodiment, the disclosure provides a method of treating cancer in a patient having (i) detectable PD-L1 expression, (ii) elevated PD-L1 expression, (iii) variability in the size of one, two, or more microsatellite repeats compared to normal cells, or (iv) any combination of (i) to (iii) by administering a compound as described herein. In another embodiment, the disclosure provides a method of treating cancer in a patient having (i) detectable PD-L1 expression, (ii) elevated PD-L1 expression, (iii) variability in the size of one, two, or more microsatellite repeats compared to normal cells, or (iv) any combination of (i) to (iii) by administering a therapeutically effective amount of a compound as described herein. In still another embodiment, the disclosure provides a method of administering a therapeutically effective amount of a compound as described herein to an individual for the treatment of cancer based on a determination of the relative amount of PD-L1 expression. In yet another embodiment, the disclosure provides a method of administering a therapeutically effective amount of a compound described herein to an individual for the treatment of cancer, the method comprising measuring PD-L1 expression and/or microsatellite instability in a sample obtained from an individual, for example by immunohistochemistry, immunophenotyping, PCR-based amplification, or other clinically validated test, and administering a therapeutically effective amount of the compound to the individual whose sample contained detectable PD-L1 expression.

### Routes of Administration

In some embodiments, pharmaceutical compositions containing a compound according to this disclosure may be in a form suitable for oral administration. Oral administration may involve swallowing the formulation thereby allowing the compound to be absorbed into the bloodstream in the gastrointestinal tract. Alternatively, oral administration may involve buccal, lingual or sublingual administration, thereby allowing the compound to be absorbed into the blood stream through oral mucosa.

In another embodiment, the pharmaceutical compositions containing a compound according to this disclosure may be in a form suitable for parenteral administration. Forms of parenteral administration include, but are not limited to, intravenous, intraarterial, intramuscular, intradermal, intraperitoneal, intrathecal, intracisternal, intracerebral, intracerebroventricular, intraventricular, and subcutaneous. Pharmaceutical compositions suitable for parenteral administration may be formulated using suitable aqueous or non-aqueous carriers. Depot injections, which are generally administered subcutaneously or intramuscularly, may also be utilized to release the compounds disclosed herein over a defined period of time.

Other routes of administration are also contemplated by this disclosure, including, but not limited to, nasal, vaginal, intraocular, rectal, topical (e.g., transdermal), and inhalation.

Particular embodiments of the present disclosure contemplate oral administration or parenteral administration.

### Pharmaceutical Compositions

The compounds of the present disclosure may be in the form of compositions suitable for administration to a subject. In general, such compositions are pharmaceutical compositions comprising a compound according to this disclosure or a pharmaceutically acceptable salt thereof and one or more pharmaceutically acceptable excipients. In certain embodiments, the compound may be present in an effective amount. The pharmaceutical compositions may be used in the methods of the present disclosure; thus, for example, the pharmaceutical compositions comprising a compound according to this disclosure can be administered to a subject in order to practice the therapeutic and prophylactic methods and uses described herein.

The pharmaceutical compositions of the present disclosure can be formulated to be compatible with the intended method or route of administration. Routes of administration may include those known in the art. Exemplary routes of administration are oral and parenteral. Furthermore, the pharmaceutical compositions may be used in combination with one or more other therapies described herein in order to treat or prevent the diseases, disorders and conditions as contemplated by the present disclosure. In one embodiment, one or more other therapeutic agents contemplated by this disclosure are included in the same pharmaceutical composition that comprises the compound according to this disclosure. In another embodiment, the one or more other therapeutical agents are in a composition that is separate from the pharmaceutical composition comprising the compound according to this disclosure.

In one aspect, the compounds described herein may be administered orally. Oral administration may be via, for example, capsule or tablets. In making the pharmaceutical compositions that include the compound of Formula (I), or a pharmaceutically acceptable salt thereof, the tablet or capsule typically includes at least one pharmaceutically acceptable excipient. Non-limiting examples of pharmaceutically acceptable excipients include lactose, dextrose, sucrose, sorbitol, mannitol, starches, gun acacia, calcium phosphate, alginates, tragacanth, gelatin, calcium silicate, microcrystalline cellulose, polyvinylpyrrolidone, polyethylene glycol, cellulose, sterile water, syrup, and methyl cellulose Additional pharmaceutically acceptable excipients include lubricating agents such as talc, magnesium stearate, and mineral oil; wetting agents; emulsifying and suspending agents; preserving agents such as methyl and propylhydroxy-benzoates.

In another aspect, the compound of Formula (I), or a pharmaceutically acceptable salt thereof, may be administered parenterally, for example by intravenous injection. A pharmaceutical composition appropriate for parenteral administration may be formulated in solution for injection or may be reconstituted for injection in an appropriate system such as a physiological solution. Such solutions may include sterile water for injection, salts, buffers, and tonicity excipients in amounts appropriate to achieve isotonicity with the appropriate physiology.

The pharmaceutical compositions described herein may be stored in an appropriate sterile container or containers. In some embodiments, the container is designed to maintain stability for the pharmaceutical composition over a given period of time.

### Administering

In general, the disclosed methods comprise administering a compound described herein, or a composition thereof, in an effective amount to a subject in need thereof. An “effective amount” with reference to a Cbl-b inhibitor of the present disclosure means an amount of the compound that is sufficient to engage the target (e.g., by inhibiting the target) at a level that is indicative of the potency of the compound. For Cbl-b, target engagement can be determined by one or more biochemical or cellular assays resulting in an EC50, ED50, EC90, IC50, or similar value which can be used as one assessment of the potency of the compound. Assays for determining target engagement include, but are not limited to, those described in the Examples. The effective amount may be administered as a single quantity or as multiple, smaller quantities (e.g., as one tablet with “x” amount, as two tablets each with “x/2” amount, etc.).

In some embodiments, the disclosed methods comprise administering a therapeutically effective amount of a compound described herein to a subject in need thereof. As used herein, the phrase “therapeutically effective amount” with reference to compound disclosed herein means a dose regimen (i.e., amount and interval) of the compound that provides the specific pharmacological effect for which the compound is administered to a subject in need of such treatment. For prophylactic use, a therapeutically effective amount may be effective to eliminate or reduce the risk, lessen the severity, or delay the onset of the disease, including biochemical, histological and/or behavioral signs or symptoms of the disease. For treatment, a therapeutically effective amount may be effective to reduce, ameliorate, or eliminate one or more signs or symptoms associated with a disease, delay disease progression, prolong survival, decrease the dose of other medication(s) required to treat the disease, or a combination thereof. With respect to cancer specifically, a therapeutically effective amount may, for example, result in the killing of cancer cells, reduce cancer cell counts, reduce tumor burden, eliminate tumors or metastasis, or reduce metastatic spread. A therapeutically effective amount may vary based on, for example, one or more of the following: the age and weight of the subject, the subject's overall health, the stage of the subject's disease, the route of administration, and prior or concomitant treatments.

Administration may comprise one or more (e.g., one, two, or three or more) dosing cycles.

In certain embodiments, the compounds contemplated by the present disclosure may be administered (e.g., orally, parenterally, etc.) at about 0.01 mg/kg to about 50 mg/kg, or about 1 mg/kg to about 25 mg/kg, of subject's body weight per day, one or more times a day, a week, or a month, to obtain the desired effect. In some embodiments, once daily administration is contemplated. In some embodiments, a suitable weight-based dose of a compound contemplated by the present disclosure is used to determine a dose that is administered independent of a subject's body weight In certain embodiments, the compounds of the present disclosure are administered (e.g., orally, parenterally, etc.) at fixed dosage levels of about 1 mg to about 1000 mg, particularly 1, 3, 5, 10, 15, 20, 25, 50, 75, 100, 150, 200, 250, 300, 400, 500, 600, 750, 800, 900, or 1000 mg, one or more times a day, a week, or a month, to obtain the desired effect.

In certain embodiments, the compound is contained in a “unit dosage form”. The phrase “unit dosage form” refers to physically discrete units, each unit containing a predetermined amount of the compound, either alone or in combination with one or more additional agents, sufficient to produce the desired effect. It will be appreciated that the parameters of a unit dosage form will depend on the particular agent and the effect to be achieved.

### Combination Therapy

The present disclosure contemplates the use of compounds disclosed herein alone or in combination with one or more additional therapy. Each additional therapy can be a therapeutic agent or another treatment modality. In embodiments comprising one or more additional therapeutic agents, each agent may target a different, but complementary, mechanism of action. The additional therapeutic agents can be small chemical molecules; macromolecules such as proteins, antibodies, peptibodies, peptides, DNA, RNA or fragments of such macromolecules; or cellular or gene therapies. Non-limiting examples of additional treatment modalities include surgical resection of a tumor, bone marrow transplant, radiation therapy, and photodynamic therapy. The use of a compound disclosed herein in combination with one or more additional therapies may have a synergistic therapeutic or prophylactic effect on the underlying disease, disorder, or condition. In addition to or alternatively, the combination therapy may allow for a dose reduction of one or more of the therapies, thereby ameliorating, reducing or eliminating adverse effects associated with one or more of the agents.

In embodiments comprising one or more additional treatment modality, the compound can be administered before, after or during treatment with the additional treatment modality. In embodiments comprising one or more additional therapeutic agent, the therapeutic agents used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a patient's course of therapy. If the combination is formulated as separate compositions, in some embodiments, the separate compositions are provided together in a kit.

### Cancer Therapies

The present disclosure contemplates the use of the compounds described herein in combination with one or more additional therapies useful in the treatment of cancer.

In some embodiments, one or more of the additional therapies is an additional treatment modality. Exemplary treatment modalities include but are not limited to surgical resection of a tumor, bone marrow transplant, radiation therapy, and photodynamic therapy.

In some embodiments, one or more of the additional therapies is a therapeutic agent. Exemplary therapeutic agents include chemotherapeutic agents, radiopharmaceuticals, hormone therapies, epigenetic modulators, ATP-adenosine axis-targeting agents, targeted therapies, signal transduction inhibitors, RAS signaling inhibitors, PI3K inhibitors, arginase inhibitors, HIF inhibitors, AXL inhibitors, PAK4 inhibitors, immunotherapeutic agents, cellular therapies, gene therapies, immune checkpoint inhibitors, and agonists of stimulatory or co-stimulatory immune checkpoints.

In some embodiments, one or more of the additional therapeutic agents is a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pemetrexed, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab paclitaxel, and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; proteasome inhibitors such as bortezomib, carfilzomib and ixazomib; topoisomerase inhibitors such as irinotecan, topotecan, etoposide, mitoxantrone, teniposide; difluoromethylornithine (DMFO); retinoic acid; esperamicins; capecitabine; anthracyclines and pharmaceutically acceptable salts, acids or derivatives of any of the above. In certain embodiments, combination therapy comprises a chemotherapy regimen that includes one or more chemotherapeutic agents. In one embodiment, combination therapy comprises a chemotherapeutic regimen comprising one or more of FOLFOX (folinic acid, fluorouracil, and oxaliplatin), FOLFIRI (e.g., folinic acid, fluorouracil, and irinotecan), FOLFIRINOX ((folinic acid, fluorouracil, irinotecan, and oxaliplatin), a taxoid (e.g., docetaxel, paclitaxel, nab-paclitaxel, etc.), and/or gemcitabine.

In some embodiments, one or more of the additional therapeutic agents is a radiopharmaceutical. A radiopharmaceutical is a form of internal radiation therapy in which a source of radiation (i.e., one or more radionuclide) is put inside a subject's body. The radiation source can be in solid or liquid form. Non-limiting examples of radiopharmaceuticals include sodium iodide 1-131, radium-223 dichloride, lobenguane iodine-131, radioiodinated vesicles (e.g., saposin C-dioleoylphosphatidylserine (SapC-DOPS) nanovesicles), various forms of brachytherapy, and various forms of targeted radionuclides. Targeted radionuclides comprise a radionuclide associated (e.g., by covalent or ionic interactions) with a molecule (“a targeting agent”) that specifically binds to a target on a cell, typically a cancer cell or an immune cell. The targeting agent may be a small molecule, a saccharide (inclusive of oligosaccharides and polysaccharides), an antibody, a lipid, a protein, a peptide, a non-natural polymer, or an aptamer.

In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen (i.e., an antigen enriched but not specific to a cancer cell), a tumor-specific antigen (i.e., an antigen with minimal to no expression in normal tissue), or a neo-antigen (i.e., an antigen specific to the genome of a cancer cell generated by non-synonymous mutations in the tumor cell genome). Non-limiting examples of targeted radionuclides include radionuclides attached to: somatostatin or peptide analogs thereof (e.g., 177Lu-Dotatate, etc.); prostate specific membrane antigen or peptide analogs thereof (e.g., 177Lu-PSMA-617, 225Ac-PSMA-617, 177Lu-PSMA-I&T, 177Lu-MIP-1095, etc.); a receptor's cognate ligand, peptide derived from the ligand, or variants thereof (e.g., 188Re-labeled VEGF125-136 or variants thereof with higher affinity to VEGF receptor, etc.); antibodies targeting tumor antigens (e.g., 1311-tositumomab, 90Y-ibritumomab tiuxetan, CAM-H2-1131 (Precirix NV), 1131-omburtamab, etc.).

In some embodiments, one or more of the additional therapeutic agents is a hormone therapy. Hormone therapies act to regulate or inhibit hormonal action on tumors. Examples of hormone therapies include, but are not limited to: selective estrogen receptor degraders such as fulvestrant, giredestrant, SAR439859, RG6171, AZD9833, rintodestrant, ZN-c5, LSZl02, D-0502, LY3484356, SHR9549; selective estrogen receptor modulators such as tamoxifen, raloxifene, 4-hydroxytamoxifen, trioxifene, keoxifene, toremifene; aromatase inhibitors such as anastrozole, exemestane, letrozole and other aromatase inhibiting 4(5)-imidazoles; gonadotropin-releasing hormone agonists such as nafarelin, triptorelrin goserelin; gonadotropin-releasing hormone antagonists such as degarelix; antiandrogens such as abiraterone, enzalutamide, apalutamide, darolutamide, flutamide, nilutamide, bicalutamide, leuprolide; 5α-reductase inhibitors such as finasteride, dutasteride; and the like. In certain embodiments, combination therapy comprises administration of a hormone or related hormonal agent. In one embodiment, combination therapy comprises administration of enzalutamide.

In some embodiments, one or more of the additional therapeutic agents is an epigenetic modulator. An epigenetic modulator alters an epigenetic mechanism controlling gene expression, and may be, for example, an inhibitor or activator of an epigenetic enzyme. Non-limiting examples of epigenetic modulators include DNA methyltransferase (DNMT) inhibitors, hypomethylating agents, and histone deacetylase (HDAC) inhibitors. In one or more embodiments, the compounds according to this disclosure are combined with DNA methyltransferase (DNMT) inhibitors or hypomethylating agents. Exemplary DNMT inhibitors include decitabine, zebularine and azacitadine. In one or more embodiments, combinations of the compounds according to this disclosure with a histone deacetylase (HDAC) inhibitor is also contemplated. Exemplary HDAC inhibitors include vorinostat, givinostat, abexinostat, panobinostat, belinostat and trichostatin A.

In some embodiments, one or more of the additional therapeutic agents is an ATP-adenosine axis-targeting agent. ATP-adenosine axis-targeting agents alter signaling mediated by adenine nucleosides and nucleotides (e.g., adenosine, AMP, ADP, ATP), for example by modulating the level of adenosine or targeting adenosine receptors. Adenosine and ATP, acting at different classes of receptors, often have opposite effects on inflammation, cell proliferation and cell death. For instance, ATP and other adenine nucleotides have antitumor effects via activation of the PS2Y1 receptor subtype, while accumulation of adenosine in the tumor microenvironment has been shown to inhibit the antitumor function of various immune cells and to augment the immunosuppressive activity of myeloid and regulatory T cells by binding to cell surface adenosine receptors. In certain embodiments, an ATP-adenosine axis-targeting agent is an inhibitor of an ectonucleotidase involved in the conversion of ATP to adenosine or an antagonist of adenosine receptor. Ectonucleotidases involved in the conversion of ATP to adenosine include the ectonucleoside triphosphate diphosphohydrolase 1 (ENTPD1, also known as CD39 or Cluster of Differentiation 39) and the ecto-5′-nucleotidase (NT5E or 5NT, also known as CD73 or Cluster of Differentiation 73). Exemplary small molecule CD73 inhibitors include CB-708, ORIC-533, LY3475070 and quemliclustat. Exemplary anti-CD39 and anti-CD73 antibodies include ES002023, TTX-030, IPH-5201, SRF-617, CPI-006, oleclumab (MEDI9447), NZV930, IPH5301, GS-1423, uliledlimab (TJD5, TJ004309), AB598, and BMS-986179. In one embodiment, the present disclosure contemplates combination of the compounds described herein with a CD73 inhibitor such as those described in WO 2017/120508, WO 2018/067424, WO 2018/094148, and WO 2020/046813. In further embodiments, the CD73 inhibitor is quemliclustat (AB680), Adenosine can bind to and activate four different G-protein coupled receptors: A₁R, A_{2A}R, A_{2B}R, and A₃R. A₂R antagonists include etrumadenant, inupadenant, taminadenant, caffeine citrate, NUV-1182, TT-702, DZD-2269, INCB-106385, EVOEXS-21546, AZD-4635, imaradenant, RVU-330, ciforadenant, PBF-509, PBF-999, PBF-1129, and CS-3005. In some embodiments, the present disclosure contemplates the combination of the compounds described herein with an A2_{A}R antagonist, an A2_{B}R antagonist, or an antagonist of A2_{A}R and A2_{B}R. In some embodiments, the present disclosure contemplates the combination of the compounds described herein with the adenosine receptor antagonists described in WO 2018/136700, WO 2018/204661, WO 2018/213377, or WO 2020/023846. In one embodiment, the adenosine receptor antagonist is etrumadenant.

In some embodiments, one or more of the additional therapeutic agents is a targeted therapy. In one aspect, a targeted therapy may comprise a targeting agent and a drug. The drug may be a chemotherapeutic agent, a radionuclide, a hormone therapy, or another small molecule drug attached to a targeting agent. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen, a tumor-specific antigen, or a neo-antigen. In some embodiments, the targeted therapy is an antibody-drug conjugate comprising an antibody and a drug, wherein the antibody specifically binds to HER2, HER3, nectin-4, or Trop-2. Specific examples of a targeted therapy comprising an antibody and a drug include but are not limited to patritumab deruxtecan, sacituzumab govitecan-hziy, telisotuzumab vedotin, and trastuzumab deruxtecan. Specific examples include but are not limited to patritumab deruxtecan and telisotuzumab vedotin. In other aspects, a targeted therapy may inhibit or interfere with a specific protein that helps a tumor grow and/or spread. Non-limiting examples of such targeted therapies include signal transduction inhibitors, RAS signaling inhibitors, inhibitors of oncogenic transcription factors, activators of oncogenic transcription factor repressors, angiogenesis inhibitors, immunotherapeutic agents, ATP-adenosine axis-targeting agents, AXL inhibitors, PARP inhibitors, PAK4 inhibitors, PI3K inhibitors, HIF-2a inhibitors, CD39 inhibitors, CD73 inhibitors, A2R antagonists, TIGIT antagonists, and PD-1 antagonists. ATP-adenosine axis-targeting agents are described above, while other agents are described in further detail below.

In some embodiments, one or more of the additional therapeutic agents is a signal transduction inhibitor. Signal transduction inhibitors are agents that selectively inhibit one or more steps in a signaling pathway (e.g., tyrosine kinase inhibitors (TKIs), serine/threonine kinase inhibitors (STKs), etc.). Signal transduction inhibitors (STIs) contemplated by the present disclosure include but are not limited to: (i) BCR-ABL kinase inhibitors (e.g., imatinib); (ii) epidermal growth factor receptor tyrosine kinase inhibitors (EGFR TKIs), including small molecule inhibitors (e.g., CLN-081, gefitinib, erlotinib, afatinib, icotinib, and osimertinib), and anti-EGFR antibodies; (iii) inhibitors of the human epidermal growth factor (HER) family of transmembrane tyrosine kinases, e.g., HER-2/neu receptor inhibitors (e.g., trastuzumab) and HER-3 receptor inhibitors; (iv) vascular endothelial growth factor receptor (VEGFR) inhibitors including small molecule inhibitors (e.g., axitinib, sunitinib and sorafenib), VEGF kinase inhibitors (e.g., lenvatinib, cabozantinib, pazopanib, tivozanib, XL092, etc.) and anti-VEGF antibodies (e.g., bevacizumab); (v) inhibitors of AKT family kinases or the AKT pathway (e.g., rapamycin); (vi) inhibitors of serine/threonine-protein kinase B-Raf (BRAF), such as, for example, vemurafenib, dabrafenib and encorafenib; (vii) inhibitors of rearranged during transfection (RET), including, for example, selpercatinib and pralsetinib; (viii) tyrosine-protein kinase Met (MET) inhibitors (e.g., tepotinib, tivantinib, cabozantinib and crizotinib); (ix) anaplastic lymphoma kinase (ALK) inhibitors (e.g., ensartinib, ceritinib, lorlatinib, crizotinib, and brigatinib); (x) inhibitors of the RAS signaling pathway (e.g., inhibitors of KRAS, HRAS, RAF, MEK, ERK) as described elsewhere herein; (xi) FLT-3 inhibitors (e.g., gilteritinib); (xii) inhibitors of Trop-2, such as, for example, the antibody drug conjugate sacituzumab govitecan-hziy; (xiii) inhibitors of the JAK/STAT pathway, e.g., JAK inhibitors including tofacitinib and ruxolitinib, or STAT inhibitors such as napabucasin; (xiv) inhibitors of NF-xB; (xv) cell cycle kinase inhibitors (e.g., flavopiridol); (xvi) phosphatidyl inositol kinase (PI3K) inhibitors; (xix) protein kinase B (AKT) inhibitors (e.g., capivasertib, miransertib); (xx) platelet-derived growth factor receptor (PDGFR) inhibitors (e.g., imatinib, sunitinib, regorafenib, avapritinib, Lenvatinib, nintedanib, famitinib, ponatinib, axitinib, repretinib, etc.); and (xxi) insulin-like growth factor receptor (IGFR) inhibitors (e.g., erlotinib, afatinib, gefitinib, osimertinib, dacomitinib); (xxii) fibroblast growth factor receptor (FGFR) inhibitors (e.g., futibatinib, erdafitinib, pemigatinib); and (xxiii) receptor tyrosine kinase KIT inhibitors (e.g., imatinib, sorafenib, sunitinib, masitinib, repretinib, avapritinib). In one or more embodiments, the additional therapeutic agent comprises an inhibitor of EGFR, VEGFR, HER-2, HER-3, BRAF, RET, MET, ALK, RAS (e.g., KRAS, MEK, ERK), FLT-3, JAK, STAT, NF-xB, PI3K, AKT, FGFR, KIT, or any combinations thereof.

In some embodiments, one or more of the additional therapeutic agents is a RAS signaling inhibitor. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, BI1701963. Direct inhibitors of RAS mutants have also been explored, and generally target the KRAS-GTP complex or the KRAS-GDP complex. Exemplary direct RAS inhibitors under development include, but are not limited to, sotorasib (AMG510), adagrasib (MRTX849), mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments the one or more RAS signaling inhibitors directly inhibit RAS mutants.

In some embodiments one or more of the additional therapeutic agents is an inhibitor of a phosphatidylinositol 3-kinase (PI3K), particularly an inhibitor of the PI3Kγ isoform. PI3Kγ inhibitors can stimulate an anti-cancer immune response through the modulation of myeloid cells, such as by inhibiting suppressive myeloid cells, dampening immune-suppressive tumor-infiltrating macrophages or by stimulating macrophages and dendritic cells to make cytokines that contribute to effective T cell responses thereby decreasing cancer development and spread. Exemplary PI3Kγ inhibitors include copanlisib, duvelisib, AT-104, ZX-101, tenalisib, eganelisib, SF-1126, AZD3458, and pictilisib. In some embodiments, the compounds according to this disclosure are combined with one or more PI3Kγ inhibitors described in WO 2020/0247496A1.

In some embodiments, one or more of the additional therapeutic agents is an inhibitor of arginase. Arginase has been shown to be either responsible for or participate in inflammation-triggered immune dysfunction, tumor immune escape, immunosuppression and immunopathology of infectious disease. Exemplary arginase compounds include CB-1158 and OAT-1746. In some embodiments, the compounds according to this disclosure are combined with one or more arginase inhibitors described in WO/2019/173188 and WO 2020/102646.

In some embodiments, one or more of the additional therapeutic agents is an inhibitor of an oncogenic transcription factor or an activator of an oncogenic transcription factor repressor. Suitable agents may act at the expression level (e.g., RNAi, siRNA, etc.), through physical degradation, at the protein/protein level, at the protein/DNA level, or by binding in an activation/inhibition pocket. Non-limiting examples include inhibitors of one or more subunit of the MLL complex (e.g., HDAC, DOT1L, BRD4, Menin, LEDGF, WDR5, KDM4C (JMJD2C) and PRMT1), inhibitors of hypoxia-inducible factor (HIF) transcription factor, and the like.

In some embodiments, one or more of the additional therapeutic agents is an inhibitor of a hypoxia-inducible factor (HIF) transcription factor, particularly HIF-2a. Exemplary HIF-2a inhibitors include belzutifan, ARO-HIF2, PT-2385, AB521, NK-2152, DFF332, and those described in WO 2021113436, WO 2021188769, and WO 2023077046. In some embodiments, the compounds according to this disclosure are combined with one or more HIF-2a inhibitors described in WO 2021188769. In some embodiments, the HIF-2a inhibitor is AB521.

In some embodiments, one or more of the additional therapeutic agents is an inhibitor of anexelekto (AXL). The AXL signaling pathway is associated with tumor growth and metastasis, and is believed to mediate resistance to a variety of cancer therapies. There are a variety of AXL inhibitors under development that also inhibit other kinases in the TAM family (i.e., TYRO3, MERTK), as well as other receptor tyrosine kinases including MET, FLT3, RON and AURORA, among others. Exemplary multikinase inhibitors include sitravatinib, rebastinib, glesatinib, gilteritinib, merestinib, cabozantinib, foretinib, BMS777607, LY2801653, S49076, and RXDX-106. AXL specific inhibitors have also been developed, e.g., small molecule inhibitors including DS-1205, SGI-7079, SLC-391, dubermatinib, bemcentinib, DP3975, and AB801; anti-AXL antibodies such as ADCT-601; and antibody drug conjugates (ADCs) such as BA3011. Another strategy to inhibit AXL signaling involves targeting AXL's ligand, GAS6. For example, batiraxcept is under development as is a Fc fusion protein that binds the GAS6 ligand thereby inhibiting AXL signaling. In some embodiments, the compounds according to this disclosure are combined with one or more AXL inhibitors described in WO2022246177, WO2022246179, or PCT/US2023/069124. In some embodiments, the AXL inhibitor is AB801.

In some embodiments, one or more of the additional therapeutic agents is an inhibitor of p21-activated kinase 4 (PAK4). PAK4 overexpression has been shown across a variety of cancer types, notably including those resistant to PD-1 therapies. While no PAK4 inhibitors have been approved, some are in development, and exhibit dual PAK4/NAMPT inhibitor activity, e.g., ATG-019 and KPT-9274. In some embodiments, the compounds according to this disclosure are combined with a PAK4 selective inhibitor. In some embodiments, the compounds according to this disclosure are combined with a PAK4/NAMPT dual inhibitor, e.g., ATG-019 or KPT-9274.

In some embodiments, one or more of the additional therapeutic agents is (i) an agent that inhibits the enzyme poly (ADP-ribose) polymerase (e.g., olaparib, niraparib and rucaparib, etc.); (ii) an inhibitor of the Bcl-2 family of proteins (e.g., venetoclax, navitoclax, etc.); (iii) an inhibitor of MCL-1; (iv) an inhibitor of the CD47-SIRPa pathway (e.g., the anti-CD47 antibody, magrolimab, etc.); (v) an isocitrate dehydrogenase (IDH) inhibitor, e.g., IDH-1 or IDH-2 inhibitor (e.g., ivosidenib, enasidenib, etc.).

In some embodiments, one or more of the additional therapeutic agents is an immunotherapeutic agent. Immunotherapeutic agents treat a disease by stimulating or suppressing the immune system. Immunotherapeutic agents useful in the treatment of cancers typically elicit or amplify an immune response to cancer cells. Non-limiting examples of suitable immunotherapeutic agents include: immunomodulators; cellular immunotherapies; vaccines; gene therapies; ATP-adenosine axis-targeting agents; immune checkpoint modulators; and certain signal transduction inhibitors. ATP-adenosine axis-targeting agents and signal transduction inhibitors are described above. Immunomodulators, cellular immunotherapies, vaccines, gene therapies, and immune checkpoint modulators are described further below.

In some embodiments, one or more of the additional therapeutic agents is an immunotherapeutic agent, more specifically a cytokine or chemokine, such as, IL-1, IL-2, IL-12, IL-18, ELC/CCL19, SLC/CCL21, MCP-1, IL-4, TNF, IL-15, MDC, IFNα, IFNβ, IFNγ, M-CSF, IL-3, GM-CSF, IL-13, and anti-IL-10; bacterial lipopolysaccharides (LPS); an organic or inorganic adjuvant that activates antigen-presenting cells and promote the presentation of antigen epitopes on major histocompatibility complex molecules agonists including, but not limited to Toll-like receptor (TLR) agonists, antagonists of the mevalonate pathway, agonists of STING; indoleamine 2,3-dioxygenase 1 (IDO1) inhibitors and immune-stimulatory oligonucleotides, as well as other T cell adjuvants.

In some embodiments, one or more of the additional therapeutic agents is an immunotherapeutic agent, more specifically a cellular therapy. Cellular therapies are a form of treatment in which viable cells are administered to a subject. In certain embodiments, one or more of the additional therapeutic agents is a cellular immunotherapy that activates or suppresses the immune system. Cellular immunotherapies useful in the treatment of cancers typically elicit or amplify an immune response. The cells can be autologous or allogenic immune cells (e.g., monocytes, macrophages, dendritic cells, NK cells, T cells, etc.) collected from one or more subject. Alternatively, the cells can be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In some embodiments, such cells may be an expanded subset of cells with distinct effector functions and/or maturation markers (e.g., adaptive memory NK cells, tumor infiltrating lymphocytes, immature dendritic cells, monocyte-derived dendritic cells, plasmacytoid dendritic cells, conventional dendritic cells (sometimes referred to as classical dendritic cells), M1 macrophages, M2 macrophages, etc.), may be genetically modified to target the cells to a specific antigen and/or enhance the cells' anti-tumor effects (e.g., engineered T cell receptor (TCR) cellular therapies, chimeric antigen receptor (CAR) cellular therapies, lymph node homing of antigen-loaded dendritic cells, etc.), may be engineered to express of have increased expression of a tumor-associated antigen, or may be any combination thereof. Non-limiting types of cellular therapies include CAR-T cell therapy, CAR-NK cell therapy, TCR therapy, and dendritic cell vaccines. Exemplary cellular immunotherapies include sipuleucel-T, tisagenlecleucel, lisocabtagene maraleucel, idecabtagene vicleucel, brexucabtagene autoleucel, and axicabtagene ciloleucel, as well as CTX110, JCAR015, JCAR017, MB-CART19.1, MB-CART20.1, MB-CART2019.1, UniCAR02-T-CD123, BMCA-CAR-T, JNJ-68284528, BNT211, andNK-92/5.28.z.

In some embodiments, one or more of the additional therapeutic agents is an immunotherapeutic agent, more specifically a gene therapy. Gene therapies comprise recombinant nucleic acids administered to a subject or to a subject's cells ex vivo in order to modify the expression of an endogenous gene or to result in heterologous expression of a protein (e.g., small interfering RNA (siRNA) agents, double-stranded RNA (dsRNA) agents, micro RNA (miRNA) agents, viral or bacterial gene delivery, etc.), as well as gene editing therapies that may or may not comprise a nucleic acid component (e.g., meganucleases, zinc finger nucleases, TAL nucleases, CRISPR/Cas nucleases, etc.), oncolytic viruses, and the like. Non-limiting examples of gene therapies that may be useful in cancer treatment include Gendicine® (rAd-p53), Oncorine® (rAD5-H101), talimogene laherparepvec, Mx-dnGl, ARO-HIF2 (Arrowhead), quaratusugene ozeplasmid (Immunogene), CTX110 (CRISPR Therapeutics), CTX120 (CRISPR Therapeutics), and CTX130 (CRISPR Therapeutics).

In some embodiments, one or more of the additional therapeutic agents is an immunotherapeutic agent, more specifically an agent that modulates an immune checkpoint. Immune checkpoints are a set of inhibitory and stimulatory pathways that directly affect the function of immune cells (e.g., B cells, T cells, NK cells, etc.). Immune checkpoints engage when proteins on the surface of immune cells recognize and bind to their cognate ligands. The present invention contemplates the use of compounds described herein in combination with agonists of stimulatory or co-stimulatory pathways and/or antagonists of inhibitory pathways. Agonists of stimulatory or co-stimulatory pathways and antagonists of inhibitory pathways may have utility as agents to overcome distinct immune suppressive pathways within the tumor microenvironment, inhibit T regulatory cells, reverse/prevent T cell anergy or exhaustion, trigger innate immune activation and/or inflammation at tumor sites, or combinations thereof.

In some embodiments, one or more of the additional therapeutic agents is an immune checkpoint inhibitor. As used herein, the term “immune checkpoint inhibitor” refers to an antagonist of an inhibitory or co-inhibitory immune checkpoint. The terms “immune checkpoint inhibitor”, “checkpoint inhibitor” and “CPI” may be used herein interchangeably. Immune checkpoint inhibitors may antagonize an inhibitory or co-inhibitory immune checkpoint by interfering with receptor-ligand binding and/or altering receptor signaling. Examples of immune checkpoints (ligands and receptors), some of which are selectively upregulated in various types of cancer cells, that can be antagonized include PD-1 (programmed cell death protein 1); PD-L1 (PD1 ligand); BTLA (B and T lymphocyte attenuator); CTLA-4 (cytotoxic T-lymphocyte associated antigen 4); TIM-3 (T cell immunoglobulin and mucin domain containing protein 3); LAG-3 (lymphocyte activation gene 3); TIGIT (T cell immunoreceptor with Ig and ITIM domains); CD276 (B7-H3), PD-L2, Galectin 9, CEACAM-1, CD69, Galectin-1, CD113, GPR56, VISTA, 2B4, CD48, GARP, PD1H, LAIR1, TIM-1, and TIM-4, and Killer Inhibitory Receptors, which can be divided into two classes based on their structural features: i) killer cell immunoglobulin-like receptors (KIRs), and ii) C-type lectin receptors (members of the type II transmembrane receptor family). Also contemplated are other less well-defined immune checkpoints that have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). [See Pardoll, (April 2012) Nature Rev. Cancer 12:252-64].

In some embodiments, an immune checkpoint inhibitor is a CTLA-4 antagonist. In further embodiments, the CTLA-4 antagonist can be an antagonistic CTLA-4 antibody. Suitable antagonistic CTLA-4 antibodies include, for example, monospecific antibodies such as ipilimumab or tremelimumab, as well as bispecific antibodies such as MED15752 and KN046.

In some embodiments, an immune checkpoint inhibitor is a PD-1 antagonist. In further embodiments, the PD-1 antagonist can be an antagonistic PD-1 antibody, small molecule or peptide. Suitable antagonistic PD-1 antibodies include, for example, monospecific antibodies such as balstilimab, budigalimab, camrelizumab, cosibelimab, dostarlimab, cemiplimab, ezabenlimab, MEDI-0680 (AMP-514; WO2012/145493), nivolumab, pembrolizumab, pidilizumab (CT-011), pimivalimab, retifanlimab, sasanlimab, spartalizumab, sintilimab, tislelizumab, toripalimab, and zimberelimab; as well as bi-specific antibodies such as LY3434172. In still further embodiments, the PD-1 antagonist can be a recombinant protein composed of the extracellular domain of PD-L2 (B7-DC) fused to the Fc portion of IgGl (AMP-224). In certain embodiments, an immune checkpoint inhibitor is zimberelimab.

In some embodiments, an immune checkpoint inhibitor is a PD-L1 antagonist. In further embodiments, the PD-L1 antagonist can be an antagonistic PD-L1 antibody. Suitable antagonistic PD-L1 antibodies include, for example, monospecific antibodies such as avelumab, atezolizumab, durvalumab, BMS-936559, and envafolimab as well as bi-specific antibodies such as LY3434172 and KN046.

In some embodiments, an immune checkpoint inhibitor is a TIGIT antagonist. In further embodiments, the TIGIT antagonist can be an antagonistic TIGIT antibody. Suitable antagonistic anti-TIGIT antibodies include monospecific antibodies such as AGEN1327, AB308 (WO2021247591), BMS 986207, COM902, domvanalimab, EOS-448, etigilimab, IBI-929, JS006, M6223, ociperlimab, SEA-TGT, tiragolumab, vibostolimab; as well as bi-specific antibodies such as AGEN1777 and AZD2936. In certain embodiments, an immune checkpoint inhibitor is an antagonistic anti-TIGIT antibody disclosed in WO2017152088 or WO2021247591. In certain embodiments, an immune checkpoint inhibitor is domvanalimab or AB308.

In some embodiments, an immune checkpoint inhibitor is a LAG-3 antagonist. In further embodiments, the LAG-3 antagonist can be an antagonistic LAG-3 antibody. Suitable antagonistic LAG-3 antibodies include, for example, BMS-986016 (WO10/19570, WO14/08218), or IMP-731 or IMP-321 (WO08/132601, WO09/44273).

In certain embodiments, an immune checkpoint inhibitor is a B7-H3 antagonist. In further embodiments, the B7-H3 antagonist is an antagonistic B7-H3 antibody. Suitable antagonist B7-H3 antibodies include, for example, enoblituzumab (WO11/109400), omburtumab, DS-7300a, ABBV-155, and SHR-A1811.

In some embodiments, one or more of the additional therapeutic agents activates a stimulatory or co-stimulatory immune checkpoint. Examples of stimulatory or co-stimulatory immune checkpoints (ligands and receptors) include B7-1, B7-2, CD28, 4-1BB (CD137), 4-1BBL, ICOS, ICOS-L, OX40, OX40L, GITR, GITRL, CD70, CD27, CD40, DR3 and CD2.

In some embodiments, an agent that activates a stimulatory or co-stimulatory immune checkpoint is a CD137 (4-1BB) agonist. In further embodiments, the CD137 agonist can be an agonistic CD137 antibody. Suitable CD137 antibodies include, for example, urelumab and utomilumab (WO12/32433). In some embodiments, an agent that activates a stimulatory or co-stimulatory immune checkpoint is a GITR agonist. In further embodiments, the GITR agonist can be an agonistic GITR antibody. Suitable GITR antibodies include, for example, BMS-986153, BMS-986156, TRX-518 (WO06/105021, WO09/009116) and MK-4166 (WO11/028683). In some embodiments, an agent that activates a stimulatory or co-stimulatory immune checkpoint is an OX40 agonist. In further embodiments, the OX40 agonist can be an agonistic OX40 antibody. Suitable OX40 antibodies include, for example, MEDI-6383, MEDI-6469, MEDI-0562, PF-04518600, GSK3174998, BMS-986178, and MOXR0916. In some embodiments, an agent that activates a stimulatory or co-stimulatory immune checkpoint is a CD40 agonist. In further embodiments, the CD40 agonist can be an agonistic CD40 antibody. In some embodiments, an agent that activates a stimulatory or co-stimulatory immune checkpoint is a CD27 agonist. In further embodiments, the CD27 agonist can be an agonistic CD27 antibody. Suitable CD27 antibodies include, for example, varlilumab.

In some embodiments, one or more of the additional therapies is an immunotherapeutic agent, more specifically an intracellular signaling molecule that influences immune cell function. For example, one or more of the additional therapies may be an inhibitor of hematopoietic progenitor kinase 1 (HPK1). HPK1 is serine/threonine kinase that functions as a negative regulator of activation signals generated by the T cell antigen receptor. As another example, one or more of the additional therapies may be an inhibitor of diacylglycerol kinase (DGK). In some embodiments, the inhibitor is a small molecule. Non-limiting examples of small molecule HPK1 inhibitors in clinical development include NDI-101150, PRj1-3024, PF-07265028, GRC 54276, CFI-402411 and BGB-15025. Non-limiting examples of small molecule DGK inhibitors include ASP1570 and BAY2965501.

In some embodiments, one or more of the additional therapeutic agents is an agent that inhibits or depletes immune-suppressive immune cells. For example, to inhibit or deplete immunosuppressive macrophages or monocytes the agent may be CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264).

In some embodiments, each additional therapeutic agent can independently be a chemotherapeutic agent, a radiopharmaceutical, a hormone therapy, an epigenetic modulator, a targeted agent, an immunotherapeutic agent, a cellular therapy, or a gene therapy. For example, in one embodiment, the present disclosure contemplates the use of the compounds described herein in combination with one or more chemotherapeutic agent and optionally one or more additional therapeutic agents, wherein each additional therapeutic agent is independently a radiopharmaceutical, a hormone therapy, a targeted agent, an immunotherapeutic agent, a cellular therapy, or a gene therapy. In another embodiment, the present disclosure contemplates the use of the compounds described herein in combination with one or more chemotherapeutic agent and optionally one or more additional therapeutic agents, wherein each additional therapeutic agent is independently a targeted agent, an immunotherapeutic agent, or a cellular therapy. In another embodiment, the present disclosure contemplates the use of the compounds described herein in combination with one or more immunotherapeutic agents and optionally one or more additional therapeutic agent, wherein each additional therapeutic agent is independently a radiopharmaceutical, a hormone therapy, a targeted agent, a chemotherapeutic agent, a cellular therapy, or a gene therapy. In another embodiment, the present disclosure contemplates the use of the compounds described herein in combination with one or more immunotherapeutic agents and optionally one or more additional therapeutic agents, wherein each additional therapeutic agent is independently a chemotherapeutic agent, a targeted agent, or a cellular therapy. In another embodiment, the present disclosure contemplates the use of the compounds described herein in combination with one or more immune checkpoint inhibitors and/or one or more ATP-adenosine axis-targeting agents, and optionally one or more additional therapeutic agents, wherein each additional therapeutic agent is independently a chemotherapeutic agent, a targeted agent, an immunotherapeutic agent, or a cellular therapy. In further embodiments of the above (a) the targeted agent can be a PI3K inhibitor, an arginase inhibitor, a HIF2a inhibitor, an AXL inhibitor, or a PAK4 inhibitor; (b) the immunotherapeutic agent is an ATP-adenosine axis-targeting agent or an immune checkpoint inhibitor; (c) the ATP-adenosine axis-targeting agent is an A2_{A}R and/or A2_{B}R antagonist, a CD73 inhibitor, or a CD39 inhibitor; (d) the ATP-adenosine axis-targeting agent is etrumadenant, quemliclustat, or AB598; (e) the immunotherapeutic agent is an anti-PD-1 antagonist antibody or an anti-TIGIT antagonist antibody; (f) the immunotherapeutic agent is zimberelimab, domvanalimab, or AB308; or (g) any combination thereof. In still further embodiments of the above, the present disclosure contemplates the use of the compounds described herein in combination with domvanalimab, etrumadenant, quemliclustat, zimberelimab, A1B308, AB521, AB598, AB610, AB801 or any combination thereof.

In some embodiments, a compound according to this disclosure is administered with one or more than one additional therapy. In some embodiments, each additional therapy is independently selected from the groups consisting of immune checkpoint inhibitors, agents that target the extracellular production of adenosine, inhibitors of HIF (e.g., a HIF-2a inhibitor), tyrosine kinase inhibitors, chemotherapeutic agents, and radiation therapy. In further embodiments, of the above (a) the one or more immune checkpoint inhibitors antagonizes at least one of PD-1, PD-L1, BTLA, LAG-3, a B7 family member, TIM-3, TIGIT, or CTLA-4; (b) the one or more immune checkpoint inhibitors comprise an immune checkpoint inhibitor that antagonizes PD-1 or PD-L1; (c) the one or more immune checkpoint inhibitors are selected from the group consisting of avelumab, atezolizumab, balstilimab, budigalimab, camrelizumab, cosibelimab, dostarlimab, durvalumab, emiplimab, envafolimab ezabenlimab, nivolumab, pembrolizumab, pidilizumab, pimivalimab, retifanlimab, sasanlimab, spartalizumab, sintilimab, tislelizumab, toripalimab, and zimberelimab; (d) the one or more immune checkpoint inhibitors comprise zimberelimab; (e) the one or more immune checkpoint inhibitors comprise an immune checkpoint inhibitor that antagonizes TIGIT; (f) the immune checkpoint inhibitor that antagonizes TIGIT is selected from the group consisting of AB308, domvanalimab, etigilimab, ociperlimab, tiragolumab, and vibostolimab; (g) the one or more immune checkpoint inhibitors comprise domvanalimab or AB308; (h) the one or more agents that target the extracellular production of adenosine are selected from the group consisting of an A₂ₐR/A_{2b}R antagonist, a CD73 inhibitor, and a CD39 inhibitor; (i) the one or more agents that target the extracellular production of adenosine are selected from the group consisting of AB598, etrumadenant, inupadenant, taminadenant, caffeine citrate, imaradenant, ciforadenant, and quemliclustat; (j) the one or more agents that target the extracellular production of adenosine are AB598, etrumadenant and/or quemliclustat; (k) the one or more inhibitors of HIF-2a are selected from the group consisting of belzutifan, ARO-HIF2, PT-2385, AB521, NK-2152, and DFF332; (1) the inhibitor of HIF-2a is AB521; (m) the one or more chemotherapeutic agents comprises a platinum-based, taxoid-based, or anthracycline-based chemotherapeutic agent; and (n) the chemotherapeutic agent is selected from the group consisting of cisplatin, carboplatin, oxaliplatin, doxorubicin, docetaxel, and paclitaxel.

Selection of the additional therapeutic agent(s) may be informed by current standard of care for a particular cancer and/or mutational status of a subject's cancer and/or stage of disease. Detailed standard of care guidelines are published, for example, by National Comprehensive Cancer Network (NCCN). See, for instance, NCCN Colon Cancer v1.2022, NCCN Hepatobiliary Cancer v1.2022, NCCN Kidney Cancer, v3.2022, NCCN NSCLC v3.2022, NCCN Pancreatic Adenocarcinoma v1.2022, NCCN Esophageal and Esophagogastric Junction Cancers v2.2022, NCCN Gastric Cancer v2.2022, Cervical Cancer v1.2022, Ovarian Cancer/Fallopian Tube Cancer/Primary Peritoneal Cancer v1.2022.

### EXPERIMENTAL

The following examples are put forth so as to provide those of ordinary skill in the art with a complete disclosure and description of how to make and use the present disclosure, and are not intended to limit the scope of what the inventors regard as their invention. Efforts have been made to ensure accuracy with respect to numbers used (e.g., amounts, temperature, etc.), but some experimental errors and deviations should be accounted for.

All reactions were performed using a Teflon-coated magnetic stir bar at the indicated temperature and were conducted under an inert atmosphere when stated. Purchased starting materials and reagents were generally used as received. Reactions were monitored by TLC (silica gel 60 with fluorescence F254, visualized with a short wave/long wave UV lamp) and/or LCMS (AGILENT® 1100 or 1200 series LCMS with UV detection at 254 or 280 nm using a binary solvent system [0.1% formic acid in MeCN/0.1% formic acid in H₂O] using one of the following columns: AGILENT® Eclipse Plus C18 [3.5 μm, 4.6 mm i.d.×100 mm], WATERS™ XSelect HSS C18 [3.5 μm, 2.1 mm i.d.×75 mm]). Flash chromatography was conducted on silica gel using an automated system (COMBIFLASH® RF+ manufactured by Teledyne ISCO), with detection wavelengths of 254 and 280 nm, and optionally equipped with an evaporative light scattering detector. Reverse phase preparative HPLC was conducted on an AGILENT® 1260 or 1290 Infinity series HPLC. Samples were eluted using a binary solvent system (MeCN/H₂O with an acid modifier as needed—for example 0.1% TFA or 0.1% formic acid) with gradient elution on a Gemini C18 110 Å column (21.2 mm i.d. xx 250 mm) with variable wavelength detection. Final compounds obtained through preparative HPLC were concentrated through lyophilization. All assayed compounds were purified to >95% purity as determined by ¹H NMR or LCMS (AGILENT® 1100 or 1200 series LCMS with UV detection at 254 or 280 nm using a binary solvent system [0.10% formic acid in MeCN/0.1% formic acid in H₂O] using one of the following columns: AGILENT® Eclipse Plus C18 [3.5 μm, 4.6 mm i.d.×100 mm], WATERS™ XSelect HSS C18 [3.5 μm, 2.1 mm i.d.×75 mm]) 1H NMR spectra were recorded on a Varian 400 MHz NMR spectrometer equipped with an Oxford AS400 magnet or a BRUKER® AVANCE NEO 400 MHz NMR. Chemical shifts (6) are reported as parts per million (ppm) relative to residual undeuterated solvent as an internal reference. The abbreviations s, br s, d, t, q, dd, dt, ddd, and m stand for singlet, broad singlet, doublet, triplet, quartet, doublet of doublets, doublet of triplets, doublet of doublet of doublets, and multiplet, respectively.

Unless indicated otherwise, temperature is in degrees Celsius (° C.), and pressure is at or near atmospheric. Standard abbreviations are used, including the following: rt=room temperature; min(s)=minute(s); h=hour(s); mg=milligram; g=gram; kg=kilogram; μL=microliter; ml or mL=milliliter; M=molar; mol=mole; mmol=millimole; N=normality; sat.=saturated; aq.=aqueous; psi=pounds per square inch; calcd=calculated; conc.=concentrated; equiv.=equivalents; DCM=dichloromethane; THF=tetrahydrofuran; EtOAc=ethyl acetate; AcOH=acetic acid; HNO₃=nitric acid; MeCN and CH₃CN=acetonitrile; NMP=N-methyl-2-pyrrolidone; DMF=N,N-dimethylformamide; DMFDMA=N,N-dimethylformamide dimethyl acetal; DMSO=dimethyl sulfoxide; CCl₄=carbon tetrachloride; CHCl₃=chloroform; iPrOH=isopropyl alcohol; EtOH=ethanol; MeOH=methanol; H₂=hydrogen gas; N₂=nitrogen gas; H₂O₂=hydrogen peroxide; DBU=1,8-diazabicyclo[5.4.0]undec-7-ene; EDC=1-ethyl-3-(3-dimethylaminopropyl)carbodiimide; HATU=1-[bis(dimethylamino)methylene]-1H-1,2,3-triazolo[4,5-b]pyridinium 3-oxid hexafluorophosphate; CDI=1,1′-carbonyldiimidazole; NIS═N-iodosuccinimide; DHP=3,4-dihydropyran; DIPEA=N,N-diisopropylethylamine; DMEDA=N,N-dimethylethane-1,2-diamine; DMEA=dimethylethanolamine; DIBAL-H=diisobutylaluminium hydride; DMP=Dess-Martin periodinane; HOBt=hydroxybenzotriazole; EDC=1-ethyl-3-(3-dimethylaminopropyl)carbodiimide; pTsOH-H₂O=p-toluenesulfonic acid monohydrate; TsCl=4-toluenesulfonyl chloride; SEM-C₁₌₂-(trimethylsilyl)ethoxymethyl chloride; MeI=iodomethane; HOBt=hydroxybenzotriazole; Et₃N=triethylamine; HC(OMe)₃=trimethyl orthoformate; HCO₂H=formic acid; AgCO₃=silver carbonate; LAH=lithium aluminum hydride; LiBF₄=lithium tetrafluoroborate; NaBH(OAc)₃=sodium triacetoxyborohydride; NaOMe=sodium methoxide; NH₄Cl=ammonium chloride; NH₃=ammonia; NaBH₄=sodium borohydride; Na₂SO₄=sodium sulfate; NaHCO₃=sodium bicarbonate; NaH=sodium hydride; NaOH=sodium hydroxide; Na₂S₂O₃=sodium thiosulfate; NaIO₄=sodium periodate; NaClO₂=sodium chlorite; NaHSO₃=sodium bisulfite; NaH₂PO₄=monosodium phosphate; MsCl=methanesulfonyl chloride; MeNCS=methyl isothiocyanate; NBS═N-bromo succinimide; KOH=potassium hydroxide; KI=potassium iodide; K₂CO₃=potassium carbonate; K₃PO₄=potassium phosphate; K₂OsO₄·2H₂O=potassium osmate(VI) dihydrate; SnCl₂=tin dichloride; Zn(CN)₂=zinc cyanide; MgSO₄=magnesium sulfate; LiOH H₂O=lithium hydroxide monohydrate; LDA=lithium diisopropylamide; CuI=copper iodide; FeCl₃=iron trichloride; HCl=hydrochloric acid; TMSCl=trimethylsilyl chloride; TMSCHN₂=trimethylsilyldiazomethane; TMSCF₂Br-(bromodifluoromethyl)trimethylsilane; TMSCF₃=trifluoromethyltrimethylsilane; TMP=2,2,6,6-tetramethylpiperidine; TEA=triethylamine; TFA=trifluoroacetic acid; Deoxo-Fluor®=bis(2-methoxyethyl)aminosulfur trifluoride; Sphos=dicyclohexyl(2′,6′-dimethoxy[1,1′-biphenyl]-2-yl)phosphane; Pd(dppf)Cl₂=[1,1′-bis(diphenylphosphino)ferrocene]palladium(II) dichloride; Pd/C=palladium on carbon; Xphos Pd G3=(2-dicyclohexylphosphino-2′,4′,6′-triisopropyl-1,1′-biphenyl)[2-(2′-amino-1,1′-biphenyl)]palladium(II) methanesulfonate; Xphos=dicyclohexyl[2′,4′,6′-tris(propan-2-yl)[1,1′-biphenyl]-2-yl]phosphane; tBuXPhos Pd G3=[(2-Di-tert-butylphosphino-2′,4′,6′-triisopropyl-1,1′-biphenyl)-2-(2′-amino-1,1′-biphenyl)]palladium(II) methanesulfonate; Pd(OAc)₂=palladium(II) acetate; Pd(PPh₃)₄=tetrakis(triphenylphosphine)palladium(0); PCy₃=tricyclohexylphosphine; Zn(CN)₂=zinc cyanide; MHz=megahertz; Hz=hertz; ppm=parts per million; ESI MS=electrospray ionization mass spectrometry; LCMS=liquid chromatography-mass spectrometry; NMR=nuclear magnetic resonance; HPLC=high pressure liquid chromatography; prep-HPLC=preparative HPLC; RP-HPLC=reverse phase HPLC.

### General Procedure 1: Synthesis of 3-[1-(3-bromophenyl)-3-methylcyclobutyl]-4-methyl-1,2,4-triazole

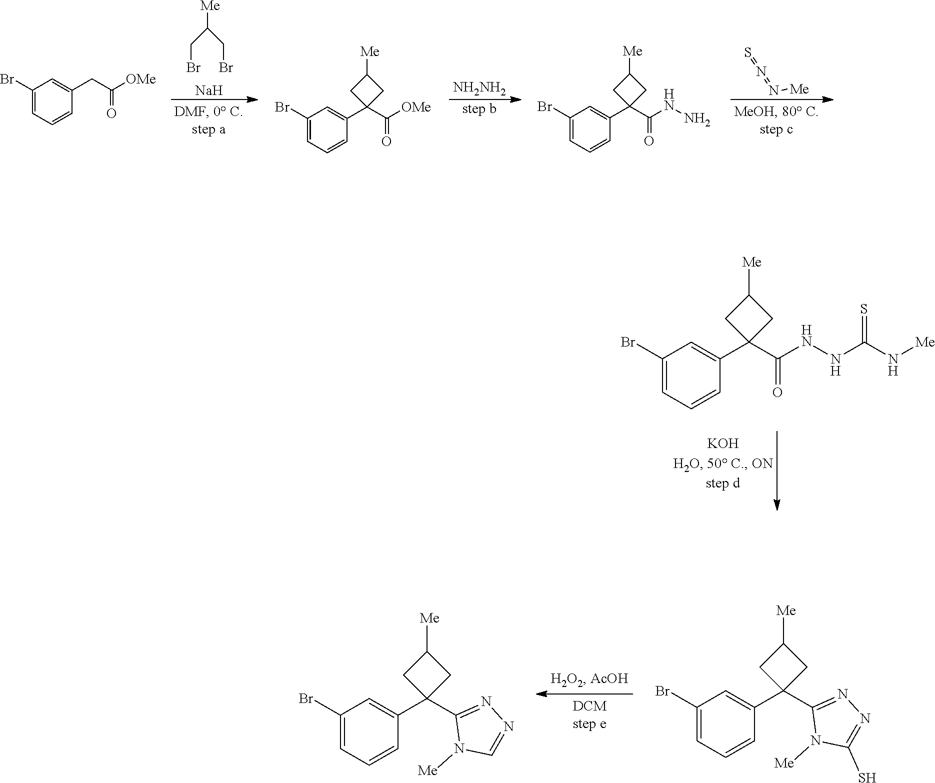

Step a: To a solution of methyl 2-(3-bromophenyl)acetate (10 g, 44.05 mmol, 1.0 equiv.) and 1,3-dibromo-2-methylpropane (9.4 g, 44.05 mmol, 1.0 equiv.) in DMF (200 mL, 0.2 M) was added sodium hydride (60% in paraffin oil) (2.16 g, 88.10 mmol, 2.0 equiv.) at 0° C. After stirring at room temperature for 16 h the reaction mixture was quenched with sat. aq. NH₄Cl solution. The organic phase was separated, and the aqueous phase was extracted with EtOAc, the combined organic phase was then washed with brine, dried over Na₂SO₄ and concentrated and the crude residue was purified by column chromatography (SiO₂, EtOAc in hexane, 0 to 20%) to give 1-(3-bromophenyl)-3-methylcyclobutane-1-carboxylate.

Step b: To a solution of the product from step a (8 g, 28.37 mmol, 1.0 equiv.) in ethanol (50 mL, 0.5 M) was added hydrazine hydrate (14 ml, 283.7 mmol, 10.0 equiv.) at rt. The mixture was stirred at 80° C. for about 16 h. After cooling down the solution to rt, the mixture was diluted with H₂O. The organic phase was separated, and the aqueous phase was extracted with EtOAc, the combined organic phase was then washed with brine, dried over Na₂SO₄ and concentrated to give 1-(3-bromophenyl)-3-methylcyclobutane-1-carbohydrazide.

Step c: To a solution of the product from step b (8 g, 28.37 mmol, 1.0 equiv.) in THE (250 mL, 0.1 M) was added methyl isothiocyanate (6.6 g, 85.11 mmol, 3.0 equiv.) at rt. The mixture was stirred at 80° C. for about 1 h. After cooling down the solution to rt, the mixture was diluted with H₂O. The organic phase was separated, and the aqueous phase was extracted with EtOAc, the combined organic phase was then washed with brine, dried over Na₂SO₄ and concentrated to give 1-[[1-(3-bromophenyl)-3-methylcyclobutanecarbonyl]amino]-3-methylthiourea.

Step d: To a solution of the product from step c (10 g, 28.17 mmol, 1.0 equiv.) in H₂O (100 mL, 0.2 M) was added KOH (7.9 g, 140.85 mmol, 5.0 equiv.) at rt. The mixture was stirred at 80° C. for about 1 h. After cooling down the solution to rt, the mixture was diluted with HCl (1 N, adjust the pH to 1-3). The mixture was filtered, and the solid cake was washed with H₂O to give 5-[1-(3-bromophenyl)-3-methylcyclobutyl]-4-methyl-1,2,4-triazole-3-thiol.

Step e: To a solution of the product from step d (9.3 g, 27.60 mmol, 1.0 equiv.) in methylene chloride (130 mL, 0.2 M) and acetic acid (18 mL) was added hydrogen peroxide (8 mL, 30%) at 0° C. The mixture was stirred at rt for about 2 h. The mixture was diluted with H₂O. The organic phase was separated, and the aqueous phase was extracted with DCM, the combined organic phase was then washed with brine, dried over Na₂SO₄ and concentrated and the crude residue was purified by column chromatography (SiO₂, MeOH in DCM, 0 to 10%) to give 3-[1-(3-bromophenyl)-3-methylcyclobutyl]-4-methyl-1,2,4-triazole as a 3:1 mixture of diastereomers.

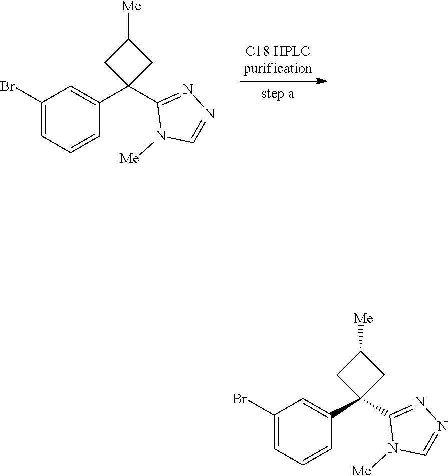 [SMILES: CC1CC(c2cccc(Br)c2)(c2nncn2C)C1 ; Cn1cnnc1[C@]1(c2cccc(Br)c2)C[C@H](C)C1]

### General Procedure 2: Synthesis of 3-((1s,3s)1-(3-bromophenyl)-3-methylcyclobutyl)-4-methyl-1,2,4-triazole

Step a: The cis/trans mixture of the product from general procedure 1 was further purified by preparative HPLC [column: Gemini 10 μm C18 110 Å; mobile phase: [water (HCO₂H, 0.1%)-MeCN]; B %: 20%-80%, 25 min] to give the target compound in >10:1 d.r. ¹H NMR (400 MHz, CDCl₃) δ 7.99 (s, 1H), 7.53 (t, J=1.8 Hz, 1H), 7.39 (dt, J=6.6, 2.1 Hz, 1H), 7.25-7.23 (m, 2H), 3.19 (s, 3H), 2.90-2.76 (m, 2H), 2.69-2.56 (m, 3H), 1.14 (d, J=5.5 Hz, 3H). ESI MS [M+H]⁺ for C₁₄H₁₇BrN, calcd 307.5, found 307.1.

### Example 1: 4-cyclopropyl-6-[3-[3-methyl-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

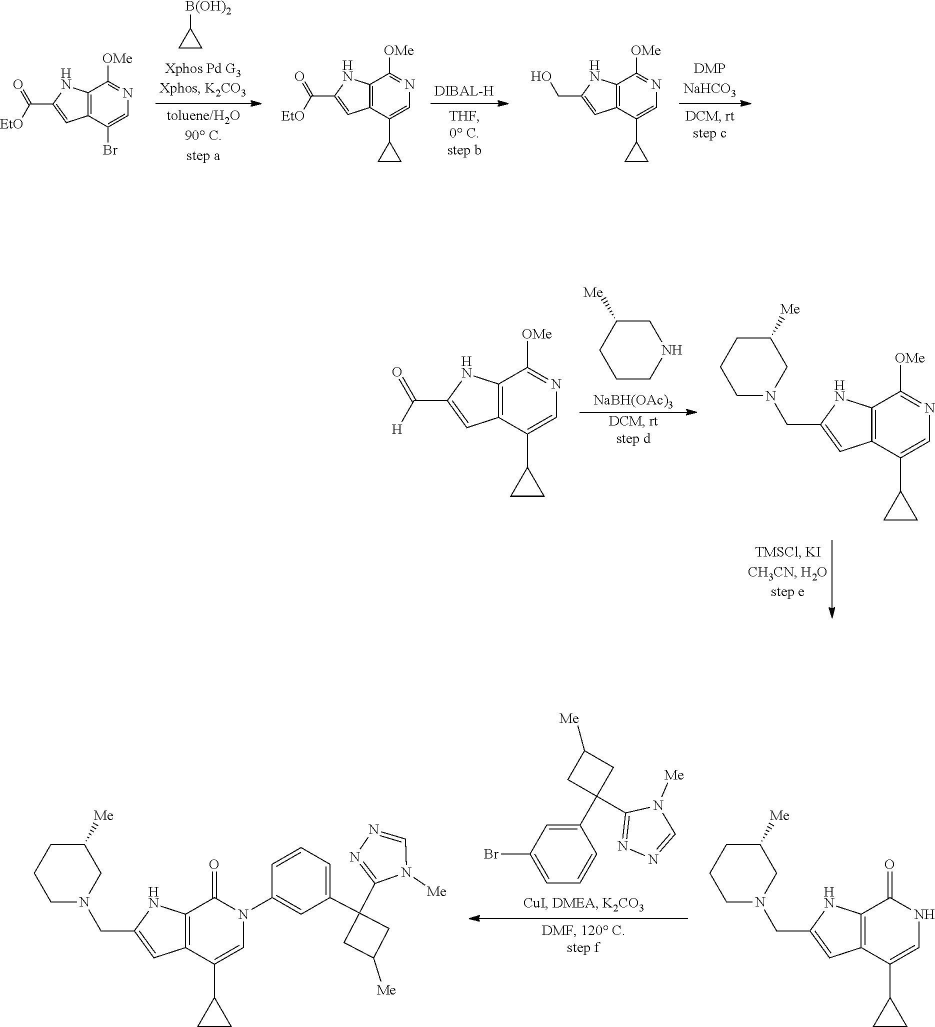

Step a: Ethyl 4-bromo-7-methoxy-1H-pyrrolo[2,3-c]pyridine-2-carboxylate (500 mg, 2.2026 mmol, 1.0 equiv.), Cyclopropylboronic acid (290 mg, 3.3039 mmol, 1.5 equiv.) and K₂CO₃ (920 mg, 6.0607 mmol, 3.0 equiv.) were dissolved in toluene/H₂O (12 mL/2 mL, 0.15 M). The mixture was purged for 2 mins under N₂. Then, Xphos Pd G3 (93 mg, 0.1101 mmol, 0.05 equiv.) and Xphos (52 mg, 0.1762 mmol, 0.08 equiv.) were added into the solution. The mixture was stirred at 90° C. for 12 h. After cooling down to rt, the reaction mixture was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexanes, 20 to 80%) to give ethyl 4-cyclopropyl-7-methoxy-1H-pyrrolo[2,3-c]pyridine-2-carboxylate.

Step b: The product of step a (540 mg, 2.08 mmol, 1.0 equiv.) was suspended in THF (10 mL, 0.2 M) followed by DIBAL-H (8.3 mL, 8.31 mmol, 4.0 equiv.) were added at 0° C. and the mixture was stirred at 0° C. for 2 h. 2 mL of 1 M aq. NaOH and 20 mL of H₂O were added, and the mixture was stirred at rt overnight. The organic phase was separated, and the aqueous layer was extracted with EtOAc. The combined organic phase was dried over Na₂SO₄, concentrated and the crude residue was purified by column chromatography (SiO₂, MeOH in DCM, 0 to 10%) to give (4-cyclopropyl-7-methoxy-1H-pyrrolo[2,3-c]pyridin-2-yl)methanol.

Step c: To the product of step b (450 mg, 2.06 mmol, 1.0 equiv.) in DCM (10 mL, 0.2 M) was added NaHCO₃ (692 mg, 8.25 mmol, 4.0 equiv.) and DMP (1.75 g, 4.12 mmol, 1.1 equiv.). The resulting mixture was stirred at rt for 3 h. The reaction was quenched with sat. aq. NH₄Cl solution, the organic phase was separated, and the aqueous layer was extracted with DCM. The combined organic phase was dried over Na₂SO₄, concentrated and the crude residue was purified by column chromatography (SiO₂, MeOH in DCM, 0 to 5%) to 4-cyclopropyl-7-methoxy-1H-pyrrolo[2,3-c]pyridine-2-carbaldehyde.

Step d: To the product of step c (100 mg, 0.46 mmol, 1.0 equiv.) in DCM (5 mL, 0.1 M) was added (S)-3-methylpiperidine hydrochloride (63 mg, 0.46 mmol, 1.0 equiv.) and DIPEA (0.16 mL, 0.93 mmol, 2.0 equiv.) and the mixture was stirred at rt for 10 mins. NaBH(OAc)₃ (150 mg, 0.69 mmol, 1.5 equiv.) was added and the mixture was stirred at rt for 12 h. The reaction was quenched with NaHCO₃ sat., the organic phase was separated, and the aqueous layer was extracted with EtOAc. The combined organic phase was dried over Na₂SO₄, concentrated and the crude residue was purified by column chromatography (SiO2, MeOH in DCM, 0 to 10%) to give 4-cyclopropyl-7-methoxy-2-[[(3S)-3-methylpiperidin-1-yl]methyl]-1H-pyrrolo[2,3-c]pyridine.

Step e: To a solution of the product from step d (122 mg, 0.4067 mmol, 1.0 equiv.) and KI (108 mg, 0.65 mmol, 1.6 equiv.) in MeCN (4 mL, 0.1 M) was added TMSCl (0.06 mL, 0.65 mmol, 1.6 equiv.) dropwise at rt. H₂O (0.1 ml) was added into the solution. The resulting mixture was stirred at 80° C. for 12 h. After cooling down the solution to rt, the mixture was quenched with H₂O. The organic phase was separated, and the aqueous phase was extracted with EtOAc, the combined organic phase was then washed with brine, dried over Na₂SO₄ and concentrated and the crude residue was purified by column chromatography (SiO₂, MeOH in DCM, 0 to 20%) to give 4-cyclopropyl-2-[[(3S)-3-methylpiperidin-1-yl]methyl]-1,6-dihydropyrrolo[2,3-c]pyridin-7-one.

Step f: To a solution of the product from step e (26 mg, 0.0911 mmol, 1.0 equiv.) and 3-[1-(3-bromophenyl)-3-methylcyclobutyl]-4-methyl-1,2,4-triazole (28 mg, 0.091 mmol, 1.0 equiv.) in DMF (2 mL, 0.05 M) was added CuI (3.5 mg, 0.018 mmol, 0.2 equiv.), DMEA (3.2 mg, 0.073 mmol, 0.8 equiv.) and K₂CO₃ (38 mg, 0.27 mmol, 3.0 equiv.). The resulting solution was stirred at 120° C. for 12 h. The reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 20%) to give a crude mixture. The resulting mixture was further was purified by preparative-HPLC to furnish the title compound as a white solid. ¹H NMR (400 MHz, CDCl₃) δ 9.62 (s, 1H), 7.99 (d, J=25.3 Hz, 1H), 7.48 (q, J=1.9 Hz, 1H), 7.43 (dd, J=12.2, 7.8 Hz, 1H), 7.36-7.30 (m, 1H), 7.23-7.17 (m, 1H), 6.73 (dd, J=3.9, 1.2 Hz, 1H), 6.38 (s, 1H), 3.61 (d, J=2.0 Hz, 2H), 3.30 (d, J=25.4 Hz, 3H), 3.17 (td, J=8.3, 2.7 Hz, 1H), 2.92-2.75 (m, 3H), 2.72-2.49 (m, 3H), 2.31 (td, J=9.3, 2.7 Hz, 1H), 1.99-1.84 (m, 2H), 1.70-1.50 (m, 5H), 1.13 (dd, J=6.1, 4.5 Hz, 3H), 0.93-0.80 (m, 5H), 0.70-0.57 (m, 2H). ESI MS [M+H]⁺ for C₃₁H₃₉N₆O, calcd 511.6, found 511.1.

### Example 2: 4-cyclopropyl-2-[[(3S)-3-methylpiperidin-1-yl]methyl]-6-[3-[1-[(4-methyl-1,2,4-triazol-3-yl)sulfanyl]ethyl]phenyl]-1H-pyrrolo[2,3-c]pyridin-7-one

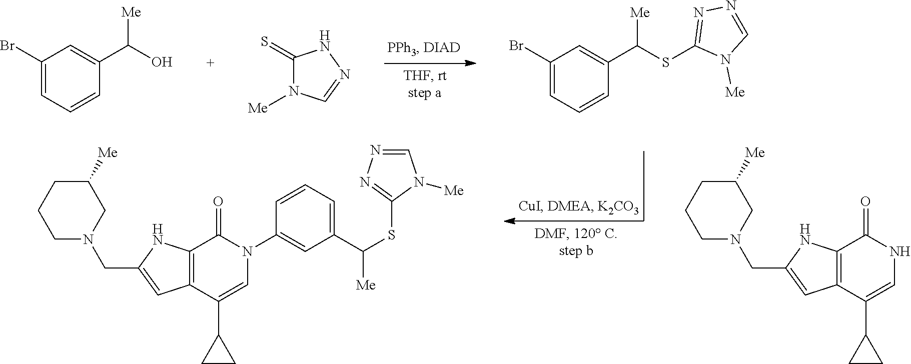 [SMILES: CC(O)c1cccc(Br)c1 ; Cn1cn[nH]c1=S ; CC(Sc1nncn1C)c1cccc(Br)c1 ; CC(Sc1nncn1C)c1cccc(-n2cc(C3CC3)c3cc(CN4CCC[C@H](C)C4)[nH]c3c2=O)c1 ; C[C@H]1CCCN(Cc2cc3c(C4CC4)c[nH]c(=O)c3[nH]2)C1]

Step a. To a solution of 1-(3-bromophenyl)ethanol (3.0 g, 14.85 mmol, 1.0 equiv.) and 4-methyl-1H-1,2,4-triazole-5-thione (2.05 g, 17.82 mmol, 1.2 equiv.) in THF (40 mL, 0.35 M) was added PPh₃ (7.80 g, 29.70 mmol, 2.0 equiv.), DIAD (6.01 g, 29.70 mmol, 2.0 equiv.). The resulting solution was stirred at rt for 12 h. The reaction was quenched with NaHCO₃ aq., the organic phase was separated, and the aqueous layer was extracted with EtOAc. The combined organic phase was dried over Na₂SO₄, concentrated and the crude residue was purified by column chromatography (MeOH in DCM, 0 to 10%) to give the title compound. ¹H NMR (400 MHz, CDCl₃) δ 8.09 (s, 1H), 7.42 (d, J=1.9 Hz, 1H), 7.38 (dt, J=7.2, 1.8 Hz, 1H), 7.19-7.10 (m, 2H), 4.74 (q, J=7.1 Hz, 1H), 3.29 (s, 3H), 1.77 (d, J=7.1 Hz, 3H). ESI MS [M+H]⁺ for C₁₁H₃BrN₃S, calcd 298.0, found 298.1.

Step b. To a solution of 4-cyclopropyl-2-[[(3S)-3-methylpiperidin-1-yl]methyl]-1,6-dihydropyrrolo[2,3-c]pyridin-7-one (21 mg, 0.074 mmol, 1.0 equiv.) and the product from step a (35 mg, 0.15 mmol, 2.0 equiv.) in DMF (2 mL, 0.05 M) was added CuI (3.0 mg, 0.01474 mmol, 0.2 equiv.), DMEA (5.2 mg, 0.059 mmol, 0.8 equiv.) and K₂CO₃ (30 mg, 0.22 mmol, 3.0 equiv.). The resulting solution was stirred at 120° C. for 12 h, the reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 20%) to give a crude mixture. The resulting mixture was further was purified by preparative HPLC to furnish the title compound. ¹H NMR (400 MHz, CDCl₃) δ 9.51 (s, 1H), 8.09 (s, 1H), 7.35 (t, J=7.8 Hz, 1H), 7.27 (m, 1H), 7.21 (d, J=7.7 Hz, 1H), 7.11 (s, 1H), 6.62 (d, J=1.2 Hz, 1H), 6.37 (s, 1H), 4.77-4.61 (m, 1H), 3.72-3.52 (m, 2H), 3.26 (d, J=29.6 Hz, 4H), 2.84-2.70 (m, 2H), 1.82 (d, J=7.1 Hz, 3H), 1.76-1.62 (m, 3H), 1.40-1.17 (m, 4H), 1.00-0.76 (m, 5H), 0.63 (q, J=5.4 Hz, 2H). ESI MS [M+H]⁺ for C₂₈H₃₅N₆₀S, calcd 503.6, found 503.1.

### Example 3: 4-cyclopropyl-6-[3-[3-methyl-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

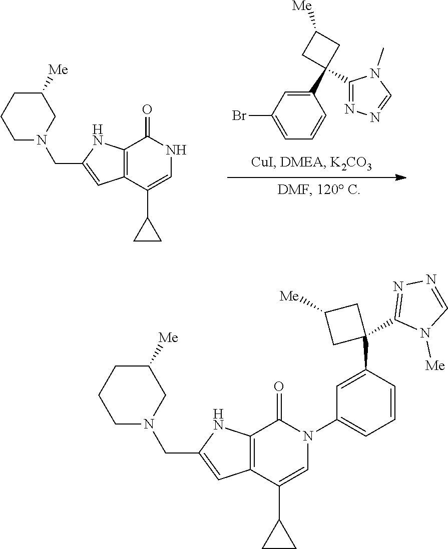

To a solution of 4-cyclopropyl-2-[[(3S)-3-methylpiperidin-1-yl]methyl]-1,6-dihydropyrrolo[2,3-c]pyridin-7-one (34 mg, 0.11 mmol, 1.0 equiv.) and 3-[1-(3-bromophenyl)-3-methylcyclobutyl]-4-methyl-1,2,4-triazole (55 mg, 0.17 mmol, 1.5 equiv.) in DMF (2 mL, 0.05 M) was added CuI (5.0 mg, 0.023 mmol, 0.2 equiv.), DMEA (4.3 mg, 0.095 mmol, 0.8 equiv.) and K₂CO₃ (50 mg, 0.4772 mmol, 3.0 equiv.). The resulting solution was stirred at 120° C. for 12 h, the reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 20%) to give a crude mixture. The resulting mixture was further was purified by preparative HPLC to furnish the title compound as a white solid. ¹H NMR (400 MHz, CDCl₃) δ 10.49 (s, 1H), 7.88 (s, 1H), 7.47-7.36 (m, 2H), 7.35-7.31 (m, 1H), 7.27 (dt, J=7.9, 1.3 Hz, 1H), 6.66 (d, J=1.2 Hz, 1H), 6.35 (d, J=1.5 Hz, 1H), 3.72 (d, J=2.0 Hz, 2H), 3.18 (s, 4H), 2.97-2.75 (m, 3H), 2.65-2.54 (m, 3H), 2.02 (td, J=10.9, 5.0 Hz, 1H), 1.85-1.55 (m, 7H), 1.06 (d, J=5.5 Hz, 3H), 0.78 (dd, J=5.4, 2.6 Hz, 5H), 0.60-0.48 (m, 2H). ESI MS [M+H]⁻ for C₃₁H₃₉N₆O, calcd 511.6, found 511.1.

### Example 4: 4-cyclopropyl-6-[3-[3-methyl-1-(4-methyl-1,2,4-triazol-3-yl)cyclobutyl]phenyl]-1H-pyrrolo[2,3-c]pyridin-7-one

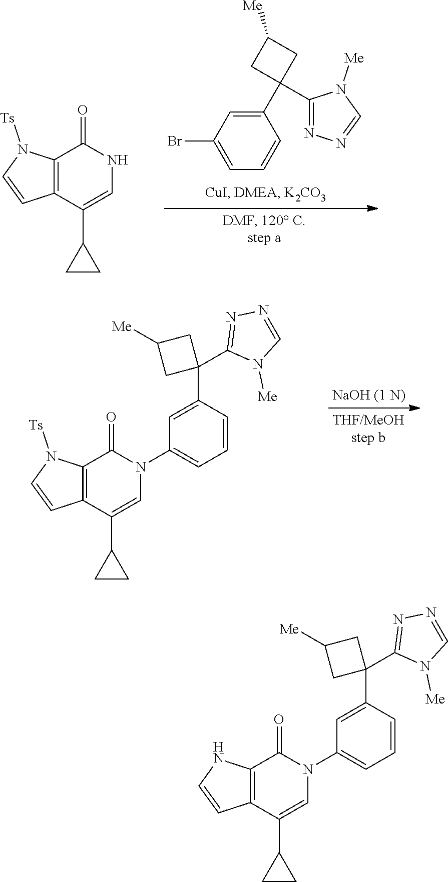

Step a: To a solution of 4-cyclopropyl-1-(4-methylphenyl)sulfonyl-6H-pyrrolo[2,3-c]pyridin-7-one (26 mg, 0.0911 mmol, 1.0 equiv.) and 3-[1-(3-bromophenyl)-3-methylcyclobutyl]-4-methyl-1,2,4-triazole (28 mg, 0.091 mmol, 1.0 equiv.) in DMF (2 mL, 0.05 M) was added CuI (3.5 mg, 0.018 mmol, 0.2 equiv.), DMEA (3.2 mg, 0.073 mmol, 0.8 equiv.) and K₂CO₃ (38 mg, 0.27 mmol, 3.0 equiv.). The resulting solution was stirred at 120° C. for 12 h. The reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 10%) to give 4-cyclopropyl-6-[3-[3-methyl-1-(4-methyl-1,2,4-triazol-3-yl)cyclobutyl]phenyl]-1-(4-methylphenyl)sulfonylpyrrolo[2,3-c]pyridin-7-one.

Step b: The product of step a (40 mg, 0.072 mmol, 1.0 equiv.) was suspended in THF/MeOH (4 mL/1 mL, 0.02 M) followed by NaOH (0.36 mL, 0.36 mmol, 5.0 equiv., 1 N) were added at rt and the mixture was stirred at rt for 2 h. The reaction was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, MeOH in DCM, 0 to 15%) to give a crude mixture. The resulting mixture was further was purified by preparative HPLC to furnish the title compound. ¹H NMR (400 MHz, CDCl₃) δ 10.19 (s, 1H), 7.96 (s, 1H), 7.48 (d, J=1.9 Hz, 1H), 7.46 (d, J=7.8 Hz, 1H), 7.36 (dt, J=8.0, 1.4 Hz, 1H), 7.29 (dd, J=2.0, 1.1 Hz, 1H), 7.25 (t, J=2.8 Hz, 1H), 6.76 (d, J=1.2 Hz, 1H), 6.58 (t, J=2.5 Hz, 1H), 3.26 (s, 3H), 2.89 (qd, J=6.6, 5.0, 2.4 Hz, 2H), 2.77-2.58 (m, 2H), 1.92 (dddd, J=13.6, 8.4, 5.3, 1.2 Hz, 1H), 1.14 (d, J=5.5 Hz, 3H), 0.96-0.82 (m, 2H), 0.74-0.59 (m, 2H). ESI MS [M+H]⁺ for C₂₄H₂₆N₅O, calcd 400.5, found 400.1.

### Example 5: 4-cyclopropyl-1-methyl-6-[3-[3-methyl-1-(4-methyl-1,2,4-triazol-3-yl)cyclobutyl]phenyl]-2-[[(3S)-3-methylpiperidin-1-yl]methyl]pyrrolo[2,3-c]pyridin-7-one

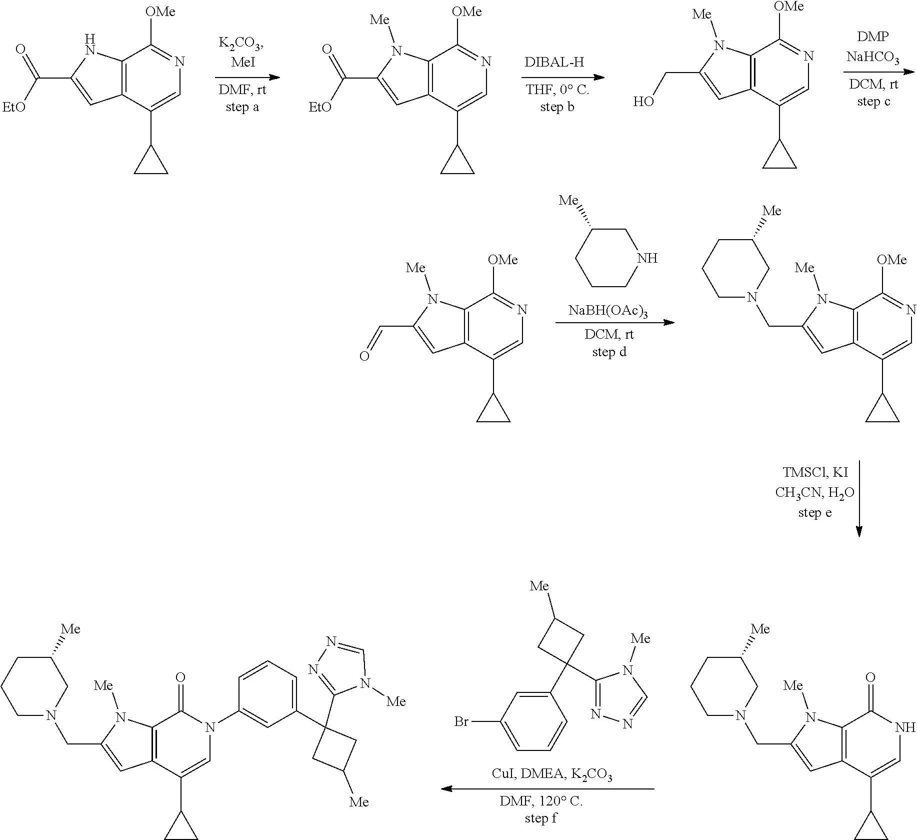

Step a. To a solution of ethyl 4-cyclopropyl-7-methoxy-1H-pyrrolo[2,3-c]pyridine-2-carboxylate (180 mg, 0.69 mmol, 1.0 equiv.) in DMF (3 mL, 0.2 M) was add K₂CO₃ (286 mg, 2.1 mmol, 3.0 equiv.) and MeI (298 mg, 2.1 mmol, 3.0 equiv.). The mixture was stirred at rt for 12 h. The reaction mixture was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, EtOAc in hexanes, 20 to 80%) to give ethyl 4-cyclopropyl-7-methoxy-1-methylpyrrolo[2,3-c]pyridine-2-carboxylate.

Step b: The product of step a (185 mg, 0.67 mmol, 1.0 equiv.) was suspended in THF (6 mL, 0.1 M) followed by DIBAL-H (2.7 mL, 2.68 mmol, 4.0 equiv.) were added at 0° C. and the mixture was stirred at 0° C. for 2 h. 2 mL of 1 M aq. NaOH and 20 mL of H₂O were added, and the mixture was stirred at rt overnight. The organic phase was separated, and the aqueous layer was extracted with EtOAc. The combined organic phase was dried over Na₂SO₄, concentrated and the crude residue was purified by column chromatography (SiO₂, MeOH in DCM, 0 to 10%) to give (4-cyclopropyl-7-methoxy-1-methylpyrrolo[2,3-c]pyridin-2-yl)methanol.

Step c: To the product of step b (170 mg, 0.73 mmol, 1.0 equiv.) in DCM (10 mL, 0.1 M) was added NaHCO₃ (308 mg, 3.66 mmol, 4.0 equiv.) and DMP (625 mg, 1.47 mmol, 2.0 equiv.). The resulting mixture was stirred at rt for 3 h. The reaction was quenched with sat. aq. NH₄Cl solution, the organic phase was separated, and the aqueous layer was extracted with DCM. The combined organic phase was dried over Na₂SO₄, concentrated and the crude residue was purified by column chromatography (SiO₂, MeOH in DCM, 0 to 5%) to 4-cyclopropyl-7-methoxy-1-methylpyrrolo[2,3-c]pyridine-2-carbaldehyde.

Step d: To the product of step c (140 mg, 0.6087 mmol, 1.0 equiv.) in DCM (5 mL, 0.1 M) was added (S)-3-methylpiperidine hydrochloride (83 mg, 0.61 mmol, 1.0 equiv.) and DIPEA (0.21 mL, 1.22 mmol, 2.0 equiv.) and the mixture was stirred at rt for 10 mins. NaBH(OAc)₃ (194 mg, 0.91 mmol, 1.5 equiv.) was added and the mixture was stirred at rt for 12 h. The reaction was quenched with NaHCO₃ sat., the organic phase was separated, and the aqueous layer was extracted with EtOAc. The combined organic phase was dried over Na₂SO₄, concentrated and the crude residue was purified by column chromatography (SiO₂, MeOH in DCM, 0 to 10%) to give 4-cyclopropyl-7-methoxy-1-methyl-2-[[(3S)-3-methylpiperidin-1-yl]methyl]pyrrolo[2,3-c]pyridine.

Step e: To a solution of the product from step d (123 mg, 0.39 mmol, 1.0 equiv.) and KI (104 mg, 0.63 mmol, 1.6 equiv.) in MeCN (4 mL, 0.1 M) was added TMSCl (0.06 mL, 0.65 mmol, 1.6 equiv.) dropwise at rt. H₂O (0.1 ml) was added into the solution. The resulting solution was stirred at 80° C. for 12 h. After cooling down the solution to rt, the mixture was quenched with H₂O. The organic phase was separated, and the aqueous phase was extracted with EtOAc, the combined organic phase was then washed with brine, dried over Na₂SO₄ and concentrated and the crude residue was purified by column chromatography (SiO₂, MeOH in DCM, 0 to 20%) to give 4-cyclopropyl-1-methyl-2-[[(3S)-3-methylpiperidin-1-yl]methyl]-6H-pyrrolo[2,3-c]pyridin-7-one.

Step f: To a solution of the product from step e (30 mg, 0.10 mmol, 1.0 equiv.) and 3-[1-(3-bromophenyl)-3-methylcyclobutyl]-4-methyl-1,2,4-triazole (46 mg, 0.15 mmol, 1.5 equiv.) in DMF (2 mL, 0.05 M) was added CuI (3.8 mg, 0.020 mmol, 0.2 equiv.), DMEA (7.1 mg, 0.080 mmol, 0.8 equiv.) and K₂CO₃ (364 mg, 0.30 mmol, 3.0 equiv.). The resulting solution was stirred at 120° C. for 12 h. The reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 10%) to give a crude mixture. The resulting mixture was further was purified by preparative HPLC to furnish the title compound as a white solid. ¹H NMR (400 MHz, CDCl₃) δ 1H NMR (400 MHz, CDCl₃) δ 7.96 (s, 1H), 7.43 (t, J=7.8 Hz, 1H), 7.38 (t, J=2.0 Hz, 1H), 7.33-7.27 (m, 2H), 6.65 (d, J=1.2 Hz, 1H), 6.37 (s, 1H), 4.19 (s, 3H), 3.56 (s, 2H), 3.24 (s, 3H), 2.94-2.76 (m, 3H), 2.73-2.59 (m, 3H), 2.02-1.79 (m, 2H), 1.73-1.45 (m, 6H), 1.14 (d, J=5.2 Hz, 3H), 0.96-0.79 (m, 7H), 0.59 (td, J=5.9, 4.2 Hz, 2H). ESI MS [M+H]⁺ for C₃₂H₄₁N₆O, calcd 525.7, found 525.1.

### Example 6: 4-cyclopropyl-6-{3-[3-methyl-1-(4-methyl-4H-1,2,4-triazol-3-yl)cyclobutyl]phenyl}-6H,7H-thieno[2,3-c]pyridin-7-one

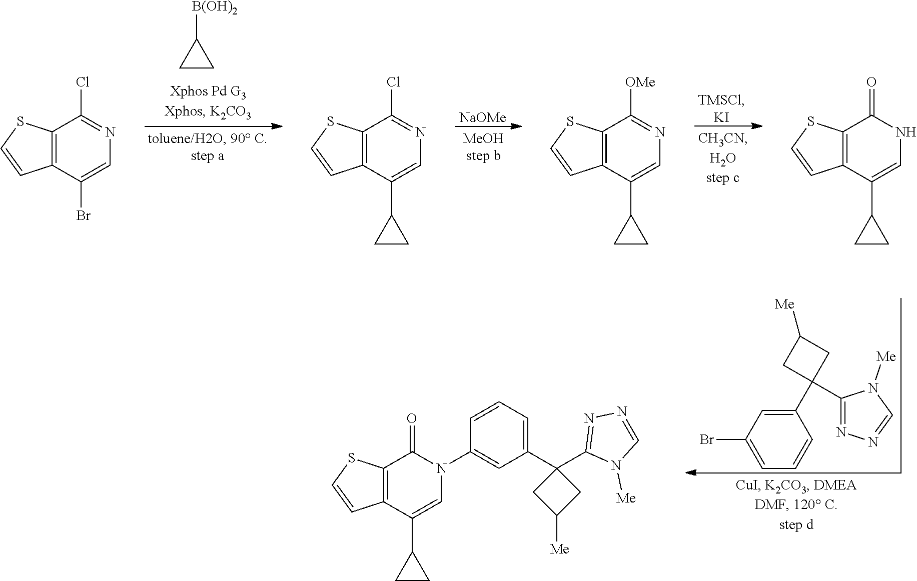

Step a: To a solution of 4-bromo-7-chlorothieno[2,3-c]pyridine (250 mg, 1.0 mmol, 1.0 equiv.), cyclopropylboronic acid (130 mg, 1.5 mmol, 1.5 equiv.) in toluene/H₂O (4 mL/1 mL, 0.2 M) was added K₂CO₃ (414 mg, 3.0 mmol, 3.0 equiv.). The mixture was purged for 2 mins under N₂. Then, Xphos Pd G3 (45 mg, 0.05 mmol, 0.05 equiv.) and Xphos (38 mg, 0.08 mmol, 0.08 equiv.) were added into the solution. The mixture was stirred at 90° C. for 12 h. After cooling down to rt, the reaction mixture was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexanes, 20 to 80%) to give 7-chloro-4-cyclopropylthieno[2,3-c]pyridine.

Step b: The product of step a (140 mg, 0.67 mmol, 1.0 equiv.) was suspended in MeOH (3 mL, 0.2 M) followed by NaOMe (55 mL, 1.0 mmol, 1.5 equiv.) were added at rt and the mixture was stirred at 80° C. for 2 h. After cooling down to rt, the reaction mixture was quenched with H₂O, the organic phase was separated, and the aqueous layer was extracted with EtOAc. The combined organic phase was dried over Na₂SO₄, concentrated and the crude residue was purified by column chromatography (SiO₂, MeOH in DCM, 0 to 10%) to give 4-cyclopropyl-7-methoxythieno[2,3-c]pyridine.

Step c: To a solution of the product from step b (83 mg, 0.40 mmol, 1.0 equiv.) and KI (108 mg, 0.65 mmol, 1.6 equiv.) in MeCN (3 mL, 0.1 M) was added TMSCl (0.065 mL, 0.65 mmol, 1.6 equiv.) dropwise at rt. H₂O (0.1 ml) was added into the solution. The resulting solution was stirred at 80° C. for 12 h. After cooling down the solution to rt, the mixture was quenched with H₂O. The organic phase was separated, and the aqueous phase was extracted with EtOAc, the combined organic phase was then washed with brine, dried over Na₂SO₄ and concentrated and the crude residue was purified by column chromatography (SiO₂, MeOH in DCM, 0 to 20%) to give 4-cyclopropyl-6H-thieno[2,3-c]pyridin-7-one.

Step d: To a solution of the product from step c (70 mg, 0.37 mmol, 1.0 equiv.) and 3-[1-(3-bromophenyl)-3-methylcyclobutyl]-4-methyl-1,2,4-triazole (168 mg, 0.055 mmol, 1.5 equiv.) in DMF (3 mL, 0.1 M) was added CuI (14 mg, 0.073 mmol, 0.2 equiv.), DMEA (26 mg, 0.29 mmol, 0.8 equiv.) and K₂CO₃ (151.7 mg, 1.10 mmol, 3.0 equiv.). The resulting solution was stirred at 120° C. for 12 h. The reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 10%) to give a crude mixture. The resulting mixture was further was purified by preparative HPLC to furnish the title compound as a white solid. ¹H NMR (400 MHz, CDCl₃) δ 8.00 (d, J=24.8 Hz, 1H), 7.78 (d, J=5.2 Hz, 1H), 7.53 (d, J=5.2 Hz, 1H), 7.50-7.42 (m, 2H), 7.39-7.31 (m, 1H), 7.26-7.20 (m, 1H), 7.00 (d, J=3.6 Hz, 1H), 3.29 (d, J=23.9 Hz, 3H), 2.88 (dt, J=10.2, 4.2 Hz, 2H), 2.74-2.54 (m, 2H), 2.31 (td, J=9.4, 2.8 Hz, 1H), 2.05-1.87 (m, 1H), 1.13 (t, J=5.4 Hz, 3H), 1.01-0.86 (m, 2H), 0.62 (q, J=5.3 Hz, 2H). ESI MS [M+H]⁺ for C₂₄H₂₅N₄₀S, calcd 417.5, found 417.1.

### Example 7: 4-cyclopropyl-6-{3-[(1r,3s)-3-methyl-1-(4-methyl-4H-1,2,4-triazol-3-yl)cyclobutyl]phenyl}-1H,6H,7H-pyrazolo[3,4-c]pyridin-7-one

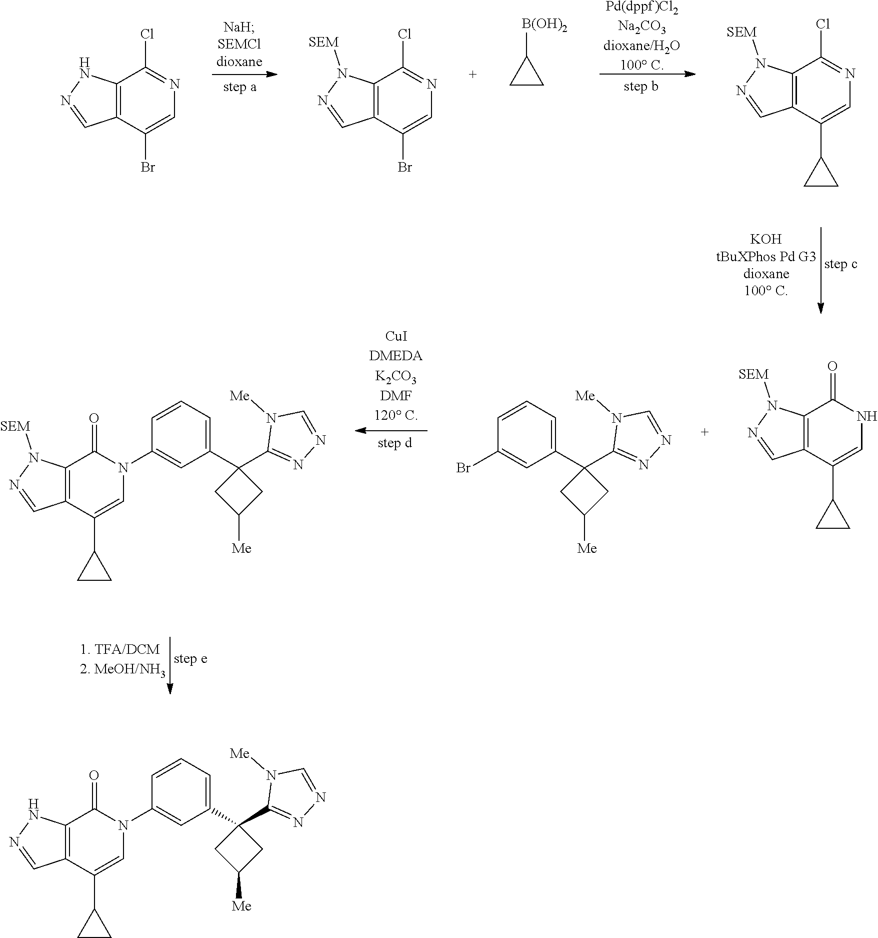

Step a: To a solution of 4-bromo-7-chloro-1H-pyrazolo[3,4-c]pyridine (1.00 g, 4.29 mmol, 1 equiv.) in dioxane (21 mL, 0.2M) was added NaH (206 mg, 60% in mineral oil, 5.15 mmol, 1.2 equiv.) at 0° C. and the mixture was stirred for 30 minutes before SEMCl (1.15 mL, 6.44 mmol, 1.5 equiv.) was added dropwise. The reaction mixture was taken out of the ice bath and left to stir at ambient temperature for one hour. The reaction was quenched by water, extracted three times with EtOAc and the combined organic layers were dried over Na₂SO₄, and concentrated. Flash column chromatography (SiO₂, 0 to 50% EtOAc/hexanes gradient) gave rise to 2-[(4-bromo-7-chloropyrazolo[3,4-c]pyridin-1-yl)methoxy]ethyl-trimethylsilane.

Step b: 2-[(4-bromo-7-chloropyrazolo[3,4-c]pyridin-1-yl)methoxy]ethyl-trimethylsilane (410 mg, 1.13 mmol), cyclopropylboronic acid (97 mg, 1.13 mmol, 1 equiv.), and Na₂CO₃ (240 mg, 2.26 mmol, 2 equiv.) were suspended water (1 mL), and dioxane (4 mL). The mixture was sparged with N₂ for 10 minutes, after which Pd(dppf)Cl₂ (83 mg, 0.113 mmol, 10%) was added and the mixture was heated to 100° C. for two hours. The reaction mixture was partitioned between EtOAc and water, the aqueous was extracted three times, and the combined organics were dried over Na₂SO₄ and concentrated. Flash column chromatography (SiO₂, 0 to 50% EtOAc/hexanes gradient) gave rise to 2-[(7-chloro-4-cyclopropylpyrazolo[3,4-c]pyridin-1-yl)methoxy]ethyl-trimethylsilane.

Step c: 2-[(7-chloro-4-cyclopropylpyrazolo[3,4-c]pyridin-1-yl)methoxy]ethyl-trimethylsilane (137 mg, 0.423 mmol, 1 equiv.) was dissolved in dioxane (4 mL). KOH (3M aq., 1.4 mL, 4.23 mmol, 10 equiv.) was added and the mixture was sparged with N₂ for 10 minutes, after which tBuXPhos Pd G3 (50 mg, 0.0635 mmol, 15%) was added and the mixture was heated to 100° C. for two hours. The reaction mixture was partitioned between EtOAc and saturated aqueous NH₄Cl, the aqueous was extracted three times, and the combined organics were dried over Na₂SO₄ and concentrated. The crude material was purified twice by flash column chromatography (first using 0 to 10% MeOH/DCM gradient, second using 0 to 100% EtOAc/DCM gradient) to give 4-cyclopropyl-1-(2-trimethylsilylethoxymethyl)-6H-pyrazolo[3,4-c]pyridin-7-one.

Step d: The reaction was performed in a similar fashion to example 1, step f.

Step e: 4-cyclopropyl-6-[3-[3-methyl-1-(4-methyl-1,2,4-triazol-3-yl)cyclobutyl]phenyl]-1-(2-trimethylsilylethoxymethyl)pyrazolo[3,4-c]pyridin-7-one (22 mg) was dissolved in dichloromethane (0.5 mL) and TFA (0.5 mL) was added. The mixture was stirred for one hour and then concentrated. To this crude material was added 7M NH₃ in methanol (1 mL) and the mixture was stirred for 16 hours. The reaction mixture was concentrated, and the crude product was purified by preparative HPLC (20% to 90% MeCN/water, 0.1% TFA) to afford the title compound. ¹H NMR (400 MHz, CDCl₃) δ 12.51 (br s, 1H), 8.02 (s, 1H), 7.97 (s, 1H), 7.50 (dd, J=8.5, 7.7 Hz, 1H), 7.47-7.42 (m, 2H), 7.29-7.27 (m, 1H), 6.78 (d, J=1.2 Hz, 1H), 3.30 (s, 3H), 2.94-2.84 (m, 2H), 2.75-2.63 (m, 3H), 1.98-1.88 (m, 1H), 1.14 (d, J=5.8 Hz, 3H), 0.98-0.91 (m, 2H), 0.75-0.68 (m, 2H). ESI MS [M+H]⁺ for C₂₃H₂₅N₆O, calcd 401.2, found 401.2.

### Example 8: 4-cyclopropyl-2-(hydroxymethyl)-6-[3-[3-methyl-1-(4-methyl-1,2,4-triazol-3-yl)cyclobutyl]phenyl]-1H-pyrrolo[2,3-c]pyridin-7-one

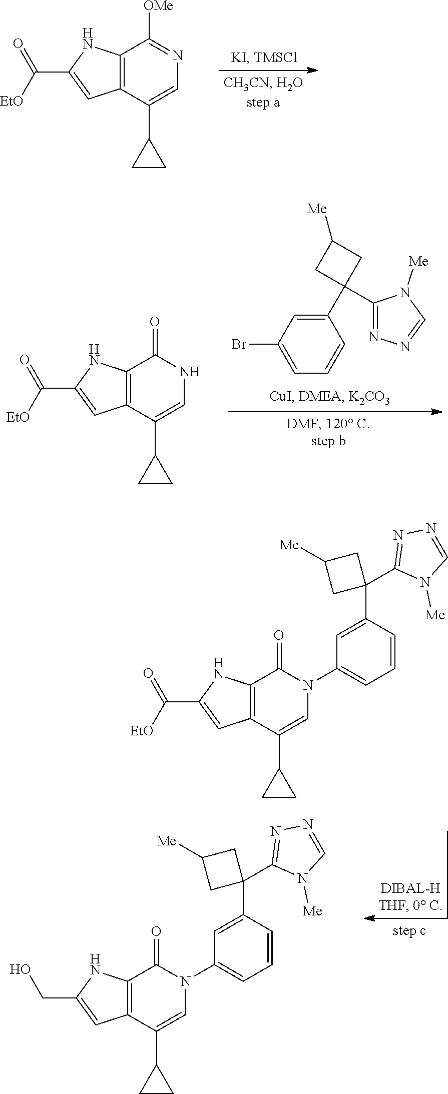

Step a: To a solution of ethyl 4-cyclopropyl-7-methoxy-1H-pyrrolo[2,3-c]pyridine-2-carboxylate (150 mg, 0.58 mmol, 1.0 equiv.) and KI (153 mg, 0.92 mmol, 1.6 equiv.) in MeCN (3 mL, 0.2 M) was added TMSCl (0.1 mL, 0.92 mmol, 1.6 equiv.) dropwise at rt. H₂O (0.1 ml) was added into the solution. The resulting solution was stirred at 80° C. for 12 h. After cooling down the solution to rt, the mixture was quenched with H₂O. The organic phase was separated, and the aqueous phase was extracted with EtOAc, the combined organic phase was then washed with brine, dried over Na₂SO₄ and concentrated and the crude residue was purified by column chromatography (SiO₂, MeOH in DCM, 0 to 20%) to give ethyl 4-cyclopropyl-7-oxo-1,6-dihydropyrrolo[2,3-c]pyridine-2-carboxylate.

Step b: To a solution of the product from step a (115 mg, 0.4675 mmol, 1.0 equiv.) and 3-[1-(3-bromophenyl)-3-methylcyclobutyl]-4-methyl-1,2,4-triazole (213 mg, 0.7012 mmol, 1.0 equiv.) in DMF (4 mL, 0.1 M) was added CuI (18.3 mg, 0.0935 mmol, 0.2 equiv.), DMEA (33.2 mg, 0.3740 mmol, 0.8 equiv.) and K₂CO₃ (193.5 mg, 1.4025 mmol, 3.0 equiv.). The resulting solution was stirred at 120° C. for 12 h. The reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 10%) to give ethyl 4-cyclopropyl-6-[3-[3-methyl-1-(4-methyl-1,2,4-triazol-3-yl)cyclobutyl]phenyl]-7-oxo-1H-pyrrolo[2,3-c]pyridine-2-carboxylate.

Step c: The product of step b (15 mg, 0.03185 mmol, 1.0 equiv.) was suspended in THF (3 mL, 0.01 M) followed by DIBAL-H (0.13 mL, 0.1274 mmol, 4.0 equiv.) were added at 0° C. and the mixture was stirred at 0° C. for 2 h. 2 mL of 1 M aq. NaOH and 20 mL of H₂O were added, and the mixture was stirred at rt overnight. The organic phase was separated, and the aqueous layer was extracted with EtOAc. The combined organic phase was dried over Na₂SO₄, concentrated and the crude residue was purified by column chromatography (SiO₂, MeOH in DCM, 0 to 20%) to give a crude mixture. The resulting mixture was further was purified by preparative HPLC to furnish the title compound as a white solid. ¹H NMR (400 MHz, CDCl₃) δ 12.40 (s, 1H), 8.02 (d, J=24.8 Hz, 1H), 7.50 (q, J=10.0, 8.9 Hz, 1H), 7.44-7.34 (m, 2H), 7.29 (s, 1H), 6.71 (d, J=5.2 Hz, 1H), 6.36 (d, J=2.0 Hz, 1H), 4.50 (s, 2H), 3.29 (d, J=14.9 Hz, 3H), 2.89 (d, J=8.7 Hz, 2H), 2.70 (d, J=7.0 Hz, 3H), 2.36 (t, J=10.4 Hz, 1H), 1.88 (td, J=8.4, 4.3 Hz, 1H), 1.22-1.07 (m, 3H), 0.91-0.76 (m, 3H), 0.65 (d, J=5.3 Hz, 2H). ESI MS [M+H]⁺ for C₂₅H₂₉N₅O₂, calcd 430.5, found 430.1.

### Example 9: 4-cyclopropyl-6-[3-[1-(4-methyl-1,2,4-triazol-3-yl)cyclobutyl]phenyl]-1H-pyrrolo[2,3-c]pyridin-7-one

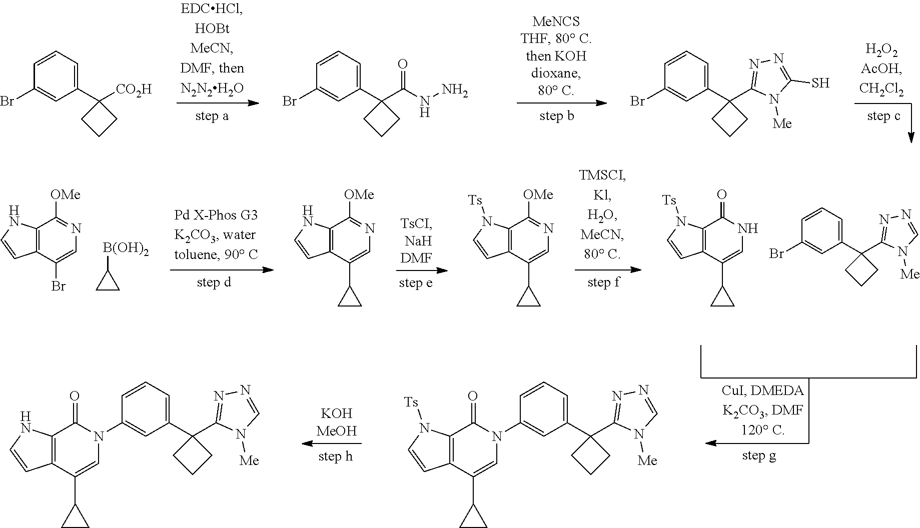

Step a: To a solution of 1-(3-bromophenyl)cyclobutane-1-carboxylic acid (2.5 g, 9.8 mmol) in acetonitrile (20 mL) and DMF (5 mL) was added HOBt (1.59 g, 11.8 mmol) and EDC HCl (2.25 g, 11.8 mmol). After 24 hours, the reaction was cooled to 0° C. and hydrazine hydrate (1.9 ml, 19.6 mmol) was added slowly. The reaction was stirred at room temperature for three hours, and upon completion was concentrated, diluted with water, and extracted with EtOAc to yield crude 1-(3-bromophenyl)cyclobutane-1-carbohydrazide.

Step b: Methyl isothiocyanate (843 μL, 12.4 mmol) was added to a solution of 1-(3-bromophenyl)cyclobutane-1-carbohydrazide (1.22 g, 4.1 mmol) in THF (40 mL) and the resulting solution was heated to 80° C. for one hour. Upon complete conversion of the starting hydrazide, aq. KOH (6N, 3.33 ml) was added, and the reaction was stirred at ambient temperature for 72 hours. Complete conversion was achieved by the addition of dioxane (40 ml) and refluxing for eight hours. On completion the reaction was concentrated, acidified with 1N HCl, and the crude product precipitated from the aqueous solution. This material was further purified by column chromatography (SiO₂, 0-10% MeOH/DCM) to afford 5-[1-(3-bromophenyl)cyclobutyl]-4-methyl-1,2,4-triazole-3-thiol as the desired product.

Step c: To a solution of 5-[1-(3-bromophenyl)cyclobutyl]-4-methyl-1,2,4-triazole-3-thiol (950 mg, 2.93 mmol) in dichloromethane (12 ml) at 0° C. was added AcOH (1.8 ml) and then hydrogen peroxide (30%, 930 μL, 8.2 mmol). The resulting solution was stirred for three hours. Upon completion, the reaction was diluted with water, extracted with dichloromethane. The organics were dried over Na₂SO₄ and concentrated to provide the desired 3-[1-(3-bromophenyl)cyclobutyl]-4-methyl-1,2,4-triazole.

Step d: Sodium hydride (60%, 548 mg, 13.7 mmol) was added carefully to a solution of 4-bromo-7-methoxy-1H-pyrrolo[2,3-c]pyridine (2.07 g, 9.1 mmol) in DMF (36.4 mL) at room temperature. Once gas evolution ceased, TsCl (2.6 g, 13.7 mmol) was added, and the reaction was stirred overnight. Product was precipitated from solution by pouring the reaction into water, and solid 4-bromo-7-methoxy-1-(4-methylphenyl)sulfonylpyrrolo[2,3-c]pyridine was isolated by filtration.

Step e: A solution of 4-bromo-7-methoxy-1-(4-methylphenyl)sulfonylpyrrolo[2,3-c]pyridine (3.4 g, 8.9 mmol), cyclopropylboronic acid (1.15 g, 13.4 mmol), and K₂CO₃ (2.46 g, 17.8 mmol) in toluene (29 mL) and H₂O (7 mL) was degassed with a stream of bubbling nitrogen for ten minutes. XPhos Pd G3 (373 mg, 0.44 mmol) was added, and the reaction was heated to 90° C. under a nitrogen atmosphere. Upon completion, the reaction was cooled to ambient temperature, filtered over CELITE®, washed with water, and the organics were concentrated to a crude mixture. This material was taken on to step f without further purification.

Step f: Crude 4-cyclopropyl-7-methoxy-1-(4-methylphenyl)sulfonylpyrrolo[2,3-c]pyridine product from step e was dissolved in MeCN (13 mL) and H₂O (13 mL). TMSCl (1.81 mL, 1.55 g, 14.3 mmol) and KI (2.28 g, 14.3 mmol) were added, and the reaction was heated to 80° C. Upon completion, the reaction was cooled to ambient temperature and partitioned between brine and dichloromethane. The organics were collected, dried over MgSO₄, and concentrated. Purification by column chromatography (SiO₂, 0-30% EtOAc/DCM) yielded the desired 4-cyclopropyl-1-(4-methylphenyl)sulfonyl-6H-pyrrolo[2,3-c]pyridin-7-one.

Step g: A solution of 4-cyclopropyl-1-(4-methylphenyl)sulfonyl-6H-pyrrolo[2,3-c]pyridin-7-one (100 mg, 0.3 mmol), 3-[1-(3-bromophenyl)cyclobutyl]-4-methyl-1,2,4-triazole (131.5 mg, 0.45 mmol), and K₂CO₃ (124 mg, 0.9 mmol) in DMF (3 mL) was degassed with a stream of bubbling nitrogen for ten minutes. CuI (12 mg, 0.06 mmol) and DMEDA (26 μL, 0.24 mmol) were added, and the reaction was heated to 120° C. overnight. On completion, the reaction was cooled to ambient temperature and poured into water. The resulting precipitate was collected and purified by column chromatography (SiO₂, 0-10% MeOH/DCM) to yield 4-cyclopropyl-1-(4-methylphenyl)sulfonyl-6-[3-[1-(4-methyl-1,2,4-triazol-3-yl)cyclobutyl]phenyl]pyrrolo[2,3-c]pyridin-7-one.

Step h: Aqueous KOH (6N, 77 μL, 0.46 mmol) was added to a solution of 4-cyclopropyl-1-(4-methylphenyl)sulfonyl-6-[3-[1-(4-methyl-1,2,4-triazol-3-yl)cyclobutyl]phenyl]pyrrolo[2,3-c]pyridin-7-one (125.4 mg, 0.23 mmol) in MeOH (1.15 mL) and stirred at room temperature. Upon completion the reaction was concentrated and purified by column chromatography (SiO₂, 0-10% MeOH/DCM) to yield the desired product, 4-cyclopropyl-6-[3-[1-(4-methyl-1,2,4-triazol-3-yl)cyclobutyl]phenyl]-1H-pyrrolo[2,3-c]pyridin-7-one. ¹H NMR (400 MHz, CDCl₃) δ 10.75 (s, 1H), 8.00 (s, 1H), 7.47-7.39 (m, 2H), 2.78-2.19 (m, 2H), 6.74-6.73 (m, 1H), 6.55-6.54 (m, 1H), 3.27 (m, 1H), 3.07-3.01 (m, 2H), 2.76-2.69 (m, 2H), 2.18-2.08 (m, 2H), 2.94-2.86 (m, 1H), 0.89-0.84 (m, 2H), 0.66-0.62 (m, 2H). ESI MS [M+H]⁺ for C₂₃H₂₃N₅O, calcd 386.2, found 386.1.

### Example 10: 4-cyclopropyl-6-[3-[5-(4-methyl-1,2,4-triazol-3-yl)spiro[2.3]hexan-5-yl]phenyl]-1H-pyrrolo[2,3-c]pyridin-7-one

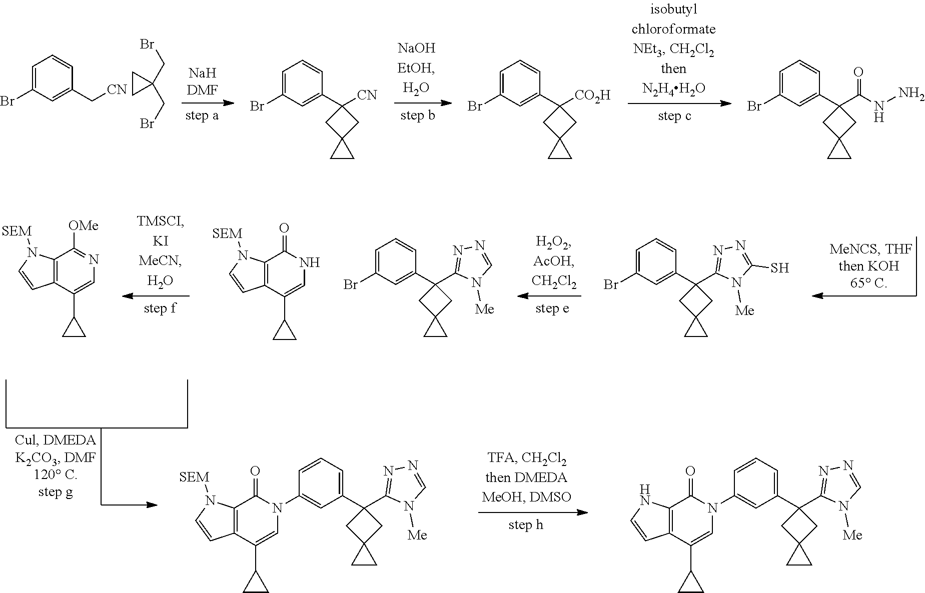

Step a: To a solution of 2-(3-bromophenyl)acetonitrile (8.62 g, 43.9 mmol) and 1,1-bis(bromomethyl)cyclopropane (10.0 g, 43.9 mmol) in DMF (44 mL) at 0° C. was added NaH, carefully and portionwise. The reaction was allowed to warm to ambient temperature, and was then heated to 65° C. overnight. Upon completion the reaction was cooled to ambient temperatures and poured into water. The crude mixture was extracted with ethyl acetate, the combined organics were washed with water, and the organics were dried, concentrated, and purified by column chromatography (SiO₂, 0-30% EtOAc/Hexanes) to yield the desired product 5-(3-bromophenyl)spiro[2.3]hexane-5-carbonitrile.

Step b: To a solution of 5-(3-bromophenyl)spiro[2.3]hexane-5-carbonitrile (6.75 g, 25.7 mmol) in EtOH (120 mL) was added NaOH (8N, 28 mL, 224 mmol), and the resulting solution was heated to 90° C. for 90 hours. On completion, the reaction was cooled to ambient temperature, concentrated to remove EtOH, and poured into 150 mL of 3 N aq. HCl. The product precipitated and was isolated by filtration to yield 5-(3-bromophenyl)spiro[2.3]hexane-5-carboxylic acid.

Step c: To a solution of 5-(3-bromophenyl)spiro[2.3]hexane-5-carboxylic acid (6.3 g, 22.4 mmol) and Et₃N (7.2 mL, 5.2 g, 52 mmol) in dichloromethane (224 mL) at 0° C. was added isobutyl chloroformate (3.3 mL, 3.46 g, 25 mmol). The resulting solution was allowed to warm to room temperature and was stirred for one hour before cooling to 0° C. and adding hydrazine hydrate (55%, 7.8 ml, 89.6 mmol). The reaction was complete after stirring at ambient temperature for 90 minutes. The solution was diluted with water and extracted with dichloromethane. The organics were washed with brine, dried over Na₂SO₄, and concentrated. The crude 5-(3-bromophenyl)spiro[2.3]hexane-5-carbohydrazide was taken on without further purification.

Step d: To a solution of 5-(3-bromophenyl)spiro[2.3]hexane-5-carbohydrazide (22.4 mmol) in THE (150 mL) was added methyl isothiocyanate (4.6 mL, 4.91 g, 67.2 mmol) and the reaction was heated to 65° C. for one hour. Upon completion, the reaction was cooled to ambient temperature and KOH (6N, 26 mL, 157 mmol) was added. The resulting solution was heated to 65° C. overnight. On completion, the reaction was cooled to ambient temperature, acidified with 3 N HCl, and extracted with ethyl acetate. The combined organics were washed with brine and dried over Na₂SO₄ before concentration to a crude mass. The crude 5-[5-(3-bromophenyl)spiro[2.3]hexan-5-yl]-4-methyl-1,2,4-triazole-3-thiol was taken on without further purification.

Step e: To a solution of 5-[5-(3-bromophenyl)spiro[2.3]hexan-5-yl]-4-methyl-1,2,4-triazole-3-thiol (22.4 mmol) in dichloromethane (112 mL) and AcOH (14.5 mL) at 0° C. was added H₂O₂ (30%, 6.6 g, 58.2 mmol) and stirred at ambient temperature overnight. On completion, the reaction pH was adjusted to >13 with 2 M aq. NaOH. The organics were collected, concentrated, and purified by column chromatography (SiO₂, 0-10% MeOH/DCM) to yield 3-[5-(3-bromophenyl)spiro[2.3]hexan-5-yl]-4-methyl-1,2,4-triazole.

Step f: To a solution of 2-[(4-cyclopropyl-7-methoxypyrrolo[2,3-c]pyridin-1-yl)methoxy]ethyl-trimethylsilane (1.29 g, 4.05 mmol) and TMSCl (821 μL, 697 mg, 6.5 mmol) in MeCN (5.8 mL) and H₂O (5.8 mL) was added KI (1.08 g, 6.5 mmol), and the reaction was stirred at ambient temperature overnight. Additional portions of TMSCl (821 μL, 697 mg, 6.5 mmol) and KI (1.08 g, 6.5 mmol) were added, and the reaction was complete after an additional 6 hours. Brine was added, the reaction was extracted with dichloromethane, and the combine organics were dried over Na₂SO₄, concentrated, and purified by flash chromatography (SiO₂, 0-40% EtOAc/DCM) to yield 4-cyclopropyl-1-(2-trimethylsilylethoxymethyl)-6H-pyrrolo[2,3-c]pyridin-7-one.

Step g: A solution of 4-cyclopropyl-1-(2-trimethylsilylethoxymethyl)-6H-pyrrolo[2,3-c]pyridin-7-one (80 mg, 0.16 mmol) and 3-[5-(3-bromophenyl)spiro[2.3]hexan-5-yl]-4-methyl-1,2,4-triazole (76 mg, 0.24 mmol) and K₂CO₃ (66.2 mg, 0.48 mmol) in DMF (1.6 ml) was degassed with a stream of bubbling nitrogen for ten minutes. CuI (6 mg, 0.03 mmol) and DMEDA (14 μL, 0.13 mmol) were added, and the reaction was heated to 120° C. overnight. On completion, the reaction was diluted with water and extracted with ethyl acetate. The combined organics were washed with water, brine, dried over Na₂SO₄, and concentrated. The crude material was purified by flash chromatography (SiO₂, 0-40% EtOAc/DCM) to yield the desired product 4-cyclopropyl-6-[3-[5-(4-methyl-1,2,4-triazol-3-yl)spiro[2.3]hexan-5-yl]phenyl]-1-(2-trimethylsilylethoxymethyl)pyrrolo[2,3-c]pyridin-7-one.

Step h: To a solution of 4-cyclopropyl-6-[3-[5-(4-methyl-1,2,4-triazol-3-yl)spiro[2.3]hexan-5-yl]phenyl]-1-(2-trimethylsilylethoxymethyl)pyrrolo[2,3-c]pyridin-7-one (106 mg, 0.2 mmol) in dichloromethane (1 mL) at ambient temperature was added TFA (1 mL), and the resulting solution was stirred at ambient temperature for three hours. Toluene was added, and the reaction was concentrated to a residue under reduced pressure. The reaction was taken up in methanol (0.5 mL) and DMSO (1.0 mL) and DMEDA (150 μL, 2.1 mmol), and the reaction was stirred overnight at ambient temperature. Upon completion, the reaction solution was purified directly by reverse phase HPLC (C18, 0-100% [0.1% TFA in MeCN]/[0.1% TFA in H₂O]) to yield 4-cyclopropyl-6-[3-[5-(4-methyl-1,2,4-triazol-3-yl)spiro[2.3]hexan-5-yl]phenyl]-1H-pyrrolo[2,3-c]pyridin-7-one. ¹H NMR (400 MHz, CDCl₃) δ 9.78 (s, 1H), 8.04 (s, 1H), 7.46 (dd, J=7.9, 7.9 Hz, 1H), 7.39-7.36 (m, 1H), 7.31 (dd, J=2.0, 2.0 Hz, 1H), 7.28-7.24 (m, 2H), 6.74 (d, J=1.2 Hz, 1H), 6.57 (dd, J=2.9, 2.2 Hz, 1H), 3.30-3.26 (m, 5H), 2.79-2.76 (m, 2H), 1.93-1.86 (m, 1H), 0.89-0.84 (m, 2H), 0.65-0.61 (m, 2H), 0.60-0.51 (m, 4H). ESI MS [M+H]⁺ for C₂₅H₂₅N₅O, calcd 412.2, found 412.2.

### Examples 11 and 12: 7-cyclopropyl-5-{3-[(1r,3s)-3-methyl-1-(4-methyl-4H-1,2,4-triazol-3-yl)cyclobutyl]phenyl}-3H,4H,5H-imidazo[4,5-c]pyridin-4-one and 7-cyclopropyl-5-{3-[(1s,3r)-3-methyl-1-(4-methyl-4H-1,2,4-triazol-3-yl)cyclobutyl]phenyl}-3H,4H,5H-imidazo[4,5-c]pyridin-4-one

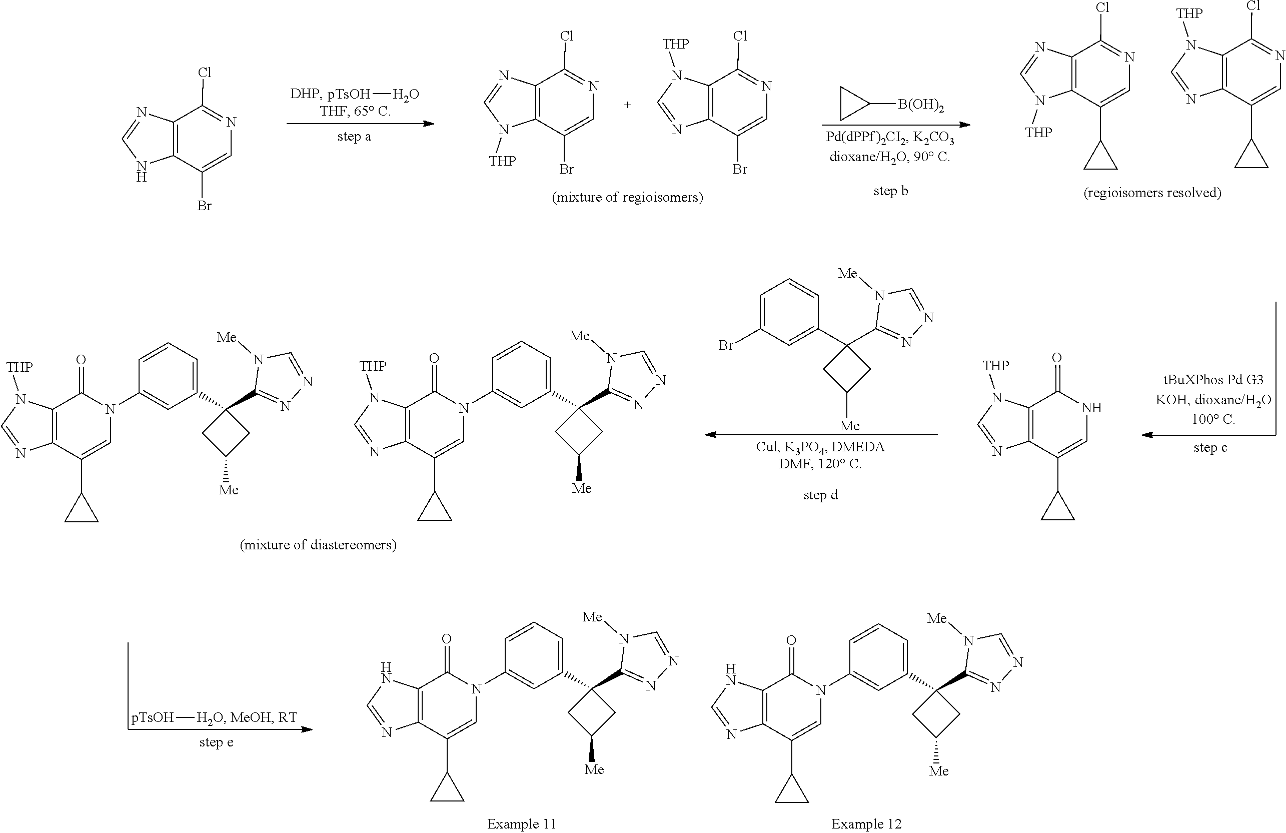

Step a: To a solution of 7-bromo-4-chloro-1H-imidazo[4,5-c]pyridine (577 mg, 2.48 mmol, 1.0 equiv.) in THF (13 mL, 0.2 M) was added DHP (1.8 mL, 19.9 mmol, 8.0 equiv.) followed by pTsOH-H₂O (48 mg, 0.25 mmol, 0.1 equiv.). The reaction mixture was heated to 65° C. and stirred for 1 hour at which point the reaction was quenched with saturated aqueous NaHCO₃ (100 mL) and extracted with EtOAc (2×50 mL). The combined organics were dried over Na₂SO₄, filtered, and concentrated under vacuum. The crude residue was purified via silica gel flash column chromatography (0 to 80% EtOAc/hexanes) to afford an inseparable ˜2:1 mixture of regioisomeric products.

Step b: A 100 mL round bottom flask was charged with the products from step a (680 mg, 2.15 mmol, 1.0 equiv.), cyclopropylboronic acid (222 mg, 2.58 mmol, 1.2 equiv.), and K₂CO₃ (890 mg, 6.44 mmol, 3.0 equiv.). The reagents were dissolved in 3:1 dioxane/water (22 mL, 0.1 M) and the reaction mixture was sparged with N₂ for 10 minutes. Pd(dppf)Cl₂ was added and the reaction was heated to 90° C. and stirred for 16 hours at which point it was quenched with half-saturated aqueous NaCl (100 mL) and extracted with EtOAc (2×50 mL). The combined organics were dried over Na₂SO₄, filtered, and concentrated under vacuum. The crude residue was purified via silica gel flash column chromatography (0 to 100% EtOAc/DCM) to afford two separable regioisomeric products.

Step c: To a solution of the product that eluted first in step b (74 mg, 0.27 mmol, 1.0 equiv.) in dioxane (2.7 mL, 0.1 M) was added 3M KOH (0.9 mL, 2.7 mmol, 10 equiv.). The reaction mixture was sparged with N₂ for 10 minutes and tBuXPhos Pd G3 (32 mg, 0.04 mmol, 0.15 equiv.) was added. The reaction was heated to 100° C. and stirred for 1 hour at which point it was quenched with saturated aqueous NH₄Cl (20 mL) and extracted with EtOAc (2×10 mL). The combined organics were dried over Na₂SO₄, filtered, and concentrated under vacuum. The crude residue was purified via silica gel flash column chromatography (0 to 20% MeOH/DCM).

Step d: To a solution of the product from step c (43 mg, 0.17 mmol, 1.0 equiv.) in DMF (3.5 mL, 0.05 M) was added 3-[1-(3-bromophenyl)-3-methylcyclobutyl]-4-methyl-1,2,4-triazole (77 mg, 0.25 mmol, 1.5 equiv.), and K₃PO₄ (106 mg, 0.50 mmol, 3.0 equiv.). The solution was stirred at room temperature for 10 minutes at which point CuI (6 mg, 0.03 mmol, 0.2 equiv.) and DMEDA (14 μL, 0.13 mmol, 0.8 equiv.) were added and the reaction was heated to 120° C. After stirring at 120° C. for 16 hours the reaction was quenched with half-saturated NaCl (20 mL) and extracted with EtOAc (2×10 mL). The combined organics were dried over Na₂SO₄, filtered, and concentrated under vacuum. The crude residue was purified via silica gel flash column chromatography (0 to 10% MeOH/DCM) to afford the product as an inseparable ˜3:1 mixture of diastereomers.

Step e: To a solution of the products from step d (25 mg, 0.05 mmol, 1.0 equiv.) in MeOH (1 mL, 0.05 M) was added pTsOH-H₂O (˜2 mg, ˜0.01 mmol, ˜0.2 equiv.). The reaction was stirred for 16 hours at room temperature at which point the reaction mixture was directly concentrated under vacuum and the crude residue was purified via reverse phase HPLC (10 to 40% MeCN/H₂O) to afford two separable diastereomeric products.

Example 11 (first eluting diastereomer, 8:1 dr): ¹H NMR (400 MHz, CDCl₃) δ 11.40 (s, 1H), 8.04 (s, 1H), 7.99 (s, 1H), 7.55-7.39 (m, 4H), 6.92 (s, 1H), 3.27 (s, 3H), 2.88 (d, J=7.5 Hz, 3H), 2.78-2.63 (m, 4H), 2.18-2.07 (m, 1H), 1.15 (d, J=5.4 Hz, 4H), 1.03-0.94 (m, 2H), 0.89-0.84 (m, 2H). ESI MS [M+H]⁺ for C₂₃H₂₄N₆O, calcd 401.2, found 401.3.

Example 12 (second eluting diastereomer, 7:1 dr): ¹H NMR (400 MHz, CDCl₃) δ 8.09 (s, 1H), 8.01 (s, 1H), 7.49 (t, J=7.6 Hz, 1H), 7.32 (d, J=7.9 Hz, 1H), 7.26-7.21 (m, 2H), 6.90 (s, 1H), 3.32 (s, 3H), 3.19 (t, J=9.0 Hz, 2H), 2.73-2.53 (m, 1H), 2.37-2.27 (m, 2H), 2.09 (td, J=8.4, 4.3 Hz, 1H), 1.14 (d, J=6.6 Hz, 4H), 1.02-0.93 (m, 2H), 0.87-0.79 (m, 2H). ESI MS [M+H]⁺ for C₂₃H₂₄N₆O, calcd 401.2, found 401.3.

### Example 13: 3-cyclopropyl-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

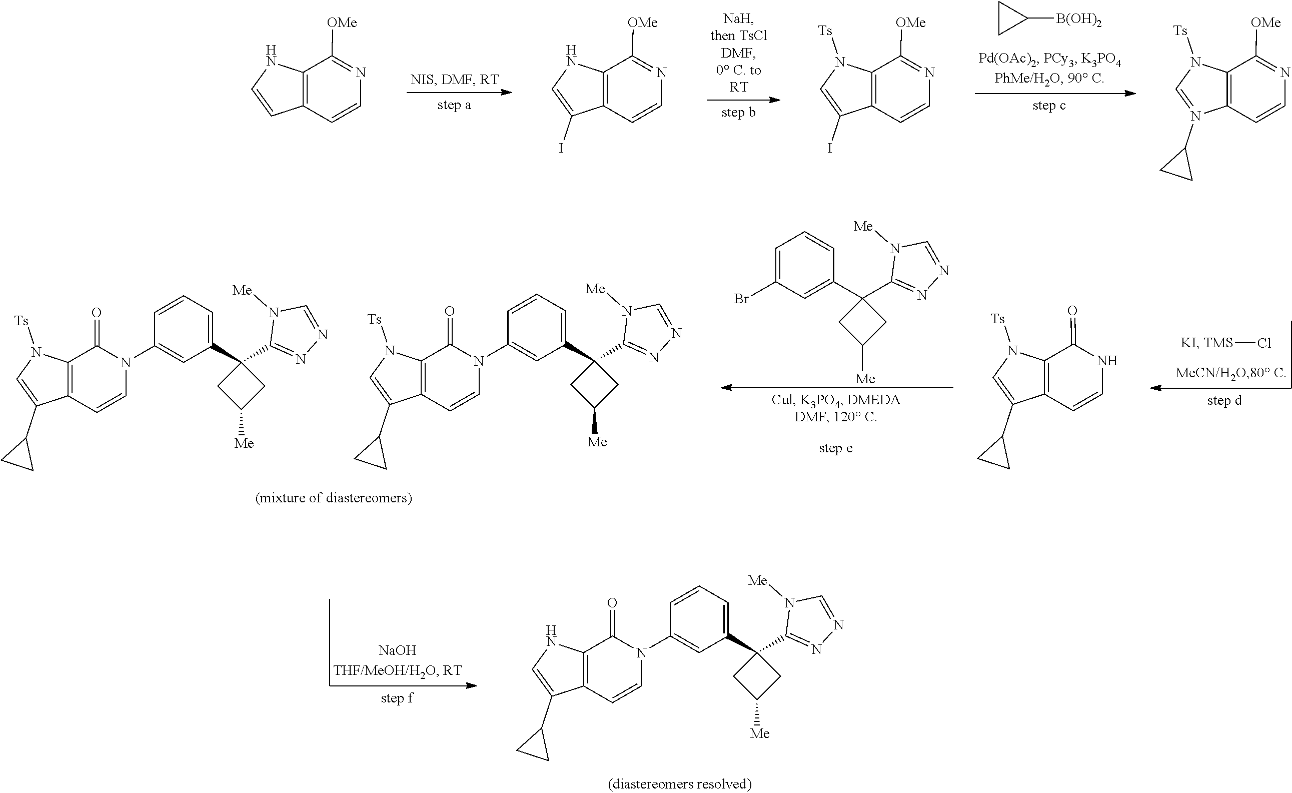

Step a: To a solution of 7-methoxy-1H-pyrrolo[2,3-c]pyridine (500 mg, 3.37 mmol, 1.0 equiv.) in DMF (7 mL, 0.5 M) was added NIS (835 mg, 3.71 mmol, 1.1 equiv.). The reaction was stirred for 20 minutes at room temperature at which point it was quenched with water (100 mL) and extracted with EtOAc (2×50 mL). The combined organics were washed with saturated aqueous NaCl (100 mL), followed by saturated aqueous Na₂S₂O₃ (100 mL), dried over Na₂SO₄, filtered, and concentrated under vacuum. The crude product was used in the subsequent step without further purification.

Step b: NaH (60% dispersion in mineral oil, 145 mg, 3.64 mmol, 1.2 equiv.) was suspended in DMF (3 mL) and the suspension was cooled to 0° C. 3-iodo-7-methoxy-1H-pyrrolo[2,3-c]pyridine (830 mg, 3.03 mmol, 1.0 equiv.) was added dropwise as a solution in DMF (3 mL, final concentration 0.5 M) and the reaction was stirred for 1 hour at 0° C. Ts-Cl (866 mg, 4.55 mmol, 1.5 equiv.) was added in a single portion and the reaction was allowed to warm to room temperature. After 3 hours the reaction was quenched with water (100 mL) and extracted with EtOAc (2×50 mL). The combined organics were washed with saturated aqueous NaCl, dried over MgSO₄, filtered, and concentrated under vacuum. The crude residue was purified via silica gel flash column chromatography (0 to 60% EtOAc/hexanes).

Step c: A 100 mL round bottomed flask was charged with 3-iodo-7-methoxy-1-(4-methylphenyl)sulfonylpyrrolo[2,3-c]pyridine (725 mg, 1.69 mmol, 1.0 equiv.), cyclopropylboronic acid (436 mg, 5.08 mmol, 3.0 equiv.), and K₃PO₄ (1.26 g, 5.93 mmol, 3.5 equiv.). The reagents were dissolved in 4:1 PhMe/H₂O (34 mL, 0.05 M) and the reaction mixture was sparged with N₂ for 10 minutes. PCy₃ (95 mg, 0.34 mmol, 0.2 equiv.) and Pd(OAc)₂ (38 mg, 0.17 mmol, 0.1 equiv.) were added to the reaction mixture and the reaction was heated to 90° C. and stirred for 16 hours. The reaction was quenched with half-saturated aqueous NaCl (100 mL) and extracted with DCM (2×50 mL). The combined organics were dried over Na₂SO₄, filtered, and concentrated. The crude residue was purified via silica gel flash column chromatography (0 to 40% EtOAc/hexanes).

Step d: To a solution of 3-cyclopropyl-7-methoxy-1-(4-methylphenyl)sulfonylpyrrolo[2,3-c]pyridine (111 mg, 0.32 mmol, 1.0 equiv.) in MeCN (2.2 mL, 0.15 M) was added KI (86 mg, 0.52 mmol, 1.6 equiv.), TMS-Cl (66 μL, 0.52 mmol, 1.6 equiv.), and H₂O (2 drops). The reaction was heated to 80° C. and stirred for 20 minutes at which point it was diluted with half-saturated aqueous NaCl (20 mL) and extracted with 3:1 CHCl₃/iPrOH (3×10 mL). The combined organics were dried over Na₂SO₄, filtered, and concentrated under vacuum. The crude product was used in the subsequent step without further purification.

Step e: An 8-dram vial was charged with 3-cyclopropyl-1-(4-methylphenyl)sulfonyl-6H-pyrrolo[2,3-c]pyridin-7-one (35 mg, 0.107 mmol, 1.0 equiv.) and 3-[1-(3-bromophenyl)-3-methylcyclobutyl]-4-methyl-1,2,4-triazole (48 mg, 0.160 mmol, 1.5 equiv.). The reagents were dissolved in DMF (1 mL, 0.1 M), K₂CO₃ (44 mg, 0.32 mmol, 3.0 equiv.) was added, and the reaction was stirred for 10 minutes at room temperature. CuI (20 mg, 0.107 mmol, 1.0 equiv.) was then added followed by DMEDA (23 μL, 0.214 mmol, 2.0 equiv.) and the reaction was heated to 120° C. After stirring for 1 hour at 120° C. the reaction was quenched with half-saturated aqueous NaCl (20 mL) and extracted with EtOAc (2×10 mL). The combined organics were dried over Na₂SO₄, filtered, and concentrated under vacuum. The crude residue was purified via silica gel flash column chromatography (0 to 20% MeOH/DCM) to afford the product as a mixture of diastereomers.

Step f: To a solution of 3-cyclopropyl-6-[3-[3-methyl-1-(4-methyl-1,2,4-triazol-3-yl)cyclobutyl]phenyl]-1-(4-methylphenyl)sulfonylpyrrolo[2,3-c]pyridin-7-one (71 mg, 0.13 mmol, 1.0 equiv.) in a 3:1 mixture of THF/MeOH (5 mL, 0.025 M) was added 1.0 M NaOH (0.64 mL, 0.64 mmol, 5.0 equiv.). The reaction was stirred for 3 hours at room temperature at which point the pH of the reaction mixture was adjusted to ˜6 with 1 M HCl and extracted with EtOAc (3×10 mL) The combined organics were dried over Na₂SO₄, filtered, and concentrated under vacuum. The crude residue was purified via reverse phase HPLC (10 to 100% MeCN/H₂O) to afford the title compound (first eluting diastereomer, 17:1 dr). ¹H NMR (400 MHz, CDCl₃) δ 9.51 (s, 1H), 7.97 (s, 1H), 7.51-7.43 (m, 2H), 7.33 (dd, J=23.0, 7.9 Hz, 2H), 7.02 (d, J=7.1 Hz, 1H), 6.96 (s, 1H), 6.72 (d, J=7.1 Hz, 1H), 3.26 (s, 3H), 2.94-2.82 (m, 2H), 2.73-2.61 (m, 3H), 1.91-1.81 (m, 1H), 1.14 (d, J=5.0 Hz, 3H), 0.90 (dd, J=8.0, 1.9 Hz, 2H), 0.62 (d, J=5.2 Hz, 2H). C₂₄H₂₅N₅O ESI MS [M+H]⁺ for C₂₄H₂₅N₅O, calcd 400.2, found 400.3.

### Example 14: 4-Cyclopropyl-7-oxo-6-{3-[(1r,3s)-3-methyl-1-(4-methyl-4H-1,2,4-triazol-3-yl)cyclobutyl]phenyl}-1H,6H,7H-pyrrolo[2,3-c]pyridine-3-carbonitrile

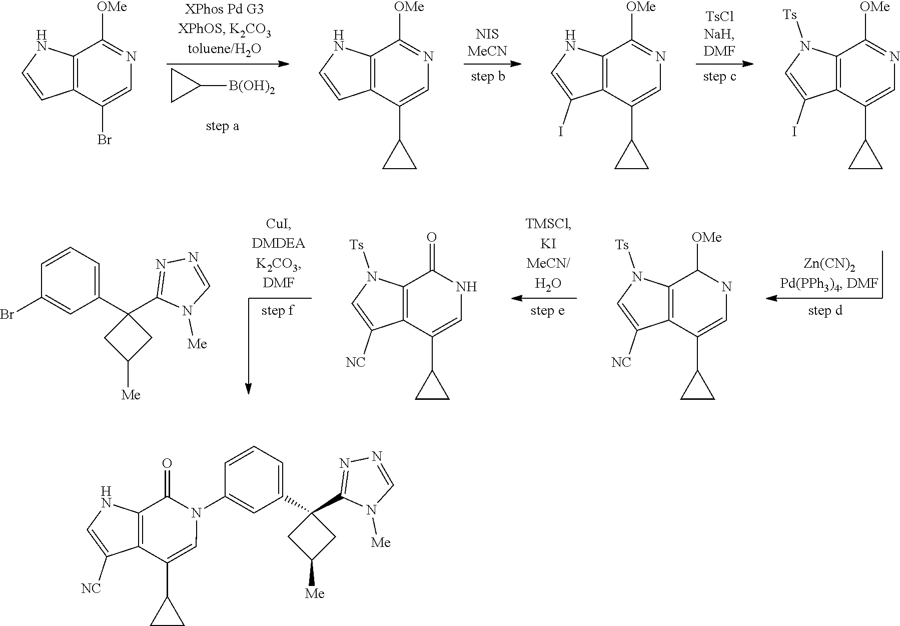

Step a: To a 500-mL round bottom flask was added 4-bromo-7-methoxy-1H-pyrrolo[2,3-c]pyridine (7.70 g, 33.9 mmol, 1.0 equiv.), cyclopropylboronic acid (3.50 g, 40.7 mmol, 1.2 equiv.), XPhos Pd G3 (0.847 g, 1.00 mmol, 3.0 mol %), XPhos (0.477 g, 1.00 mmol, 3.0 mol %), K₂CO₃ (11.7 g, 84.7 mmol, 2.5 equiv.), toluene (170 mL) and H₂O (34 mL). The resulting mixture was heated at reflux for overnight. After cooling down to room temperature, the organic phase was separated, and the aqueous layer was extracted with EtOAc. The combined organic phase was dried over Na₂SO₄, concentrated and the crude residue was purified by column chromatography (SiO₂, EtOAc in hexanes, 0 to 15%) to give 4-cyclopropyl-7-methoxy-1H-pyrrolo[2,3-c]pyridine.

Step b: To a solution of the product from step a (2.63 g, 14.0 mmol, 1.0 equiv.) in MeCN (47 mL) was added N-iodosuccinimide (3.30 g, 14.7 mmol, 1.05 equiv.) at once. The resulting mixture was stirred for 10 min when LCMS showed the completion of the iodation. The mixture was concentrated and the crude was purified by column chromatography (SiO₂, EtOAc in hexanes, 0 to 30%) to give 4-cyclopropyl-3-iodo-7-methoxy-1H-pyrrolo[2,3-c]pyridine.

Step c: To a solution of the product from step b (4.00 g, 12.7 mmol, 1.0 equiv.) in DMF (40 mL) was added NaH (60 wt % in mineral oil, 0.612 g, 15.3 mmol, 1.2 equiv.) at 0° C. The resulting mixture was stirred at this temperature for 10 min before the addition of 4-toluenesulfonyl chloride (2.91 g, 15.3 mmol, 1.2 equiv.). The reaction mixture was then raised to room temperature and stirred for overnight. The reaction was then quenched with water and diluted with EtOAc. The organic phase was separated and washed with water twice. The organic solution was then washed with brine, dried over Na₂SO₄ and concentrated. The crude residue was purified by column chromatography (SiO₂, EtOAc in hexanes, 0 to 15%) to give 4-cyclopropyl-3-iodo-7-methoxy-1-(4-methylphenyl)sulfonylpyrrolo[2,3-c]pyridine.

Step d: To a 40-mL vial was added the product from step c (137 mg, 0.29 mmol, 1.0 equiv.), Zn(CN)₂ (51.1 mg, 0.44 mmol, 1.5 equiv.), Pd(PPh₃)₄ (33.8 mg, 0.029 mmol, 10 mol %) and DMF (2.0 mL). The resulting mixture was heated under N₂ at 100° C. for 3 h when LCMS showed a completion of the cyanation. After cooling to room temperature, the reaction mixture was diluted with EtOAc and washed with water twice. The organic phase was then washed with brine, dried over Na₂SO₄ and concentrated. The crude residue was purified by column chromatography (SiO₂, EtOAc in hexanes, 0 to 20%) to give the nitrile product.

Step e: To a mixture of the product from step d (72.2 mg, 0.20 mmol, 1.0 equiv.) in MeCN/H₂O (4:1 v/v, 2.0 mL) was added TMSCl (34.8 mg, 0.32 mmol, 1.6 equiv.) and KI (53.1 mg, 0.32 mmol, 1.6 equiv.). The resulting mixture was heated at 80° C. for 2 h when LCMS showed a completion of the demethylation. After cooling to room temperature, the reaction mixture was concentrated to dryness. The crude residue was purified by column chromatography (SiO₂, MeOH in DCM, 0 to 10%) to give the desired product.

Step f: To a solution of the product from step e (52.2 mg, 0.15 mmol, 1.0 equiv.), 3-[1-(3-bromophenyl)-3-methylcyclobutyl]-4-methyl-1,2,4-triazole (diastereomeric mixture in 3:1 ratio, 68.2 mg, 0.22 mmol, 1.5 equiv.) in DMF (1.5 mL) was added CuI (5.7 mg, 0.030 mmol, 20 mol %), N,N′-dimethylethylenediamine (5.3 mg, 0.060 mmol, 40 mol %) and K₂CO₃ (58.6 mg, 0.45 mmol, 3.0 equiv.). The resulting mixture was heated at 110° C. for overnight when LCMS showed a full conversion to the desired product with a clean detosylation. After cooling to the room temperature, the reaction mixture was diluted with EtOAc and washed with water twice. The organic phase was then washed with brine, dried over Na₂SO₄ and concentrated. The crude residue was purified by column chromatography (SiO₂, MeOH in DCM, 0 to 10%) and further purified by HPLC to afford the title compound as a single diastereomer. ¹H NMR (400 MHz, CDCl₃) δ 8.23-7.97 (m, 1H), 7.73 (s, 1H), 7.58-7.44 (m, 3H), 7.38 (s, 1H), 7.26 (s, 1H), 6.86 (s, 1H), 3.28 (s, 3H), 2.94-2.81 (m, 2H), 2.78-2.59 (m, 3H), 2.16 (td, J=8.4, 4.3 Hz, 1H), 1.16 (d, J=5.3 Hz, 3H), 1.06-0.97 (m, 2H), 0.71-0.62 (m, 2H). ESI MS [M+H]⁺ for C₂₅H₂₄N₆O, calcd 425.2, found 425.2.

### Example 15: 4-Cyclopropyl-6-{3-[3-methyl-1-(4-methyl-4H-1,2,4-triazol-3-yl)cyclobutyl]phenyl}-3-{[(3S′)-3-methylpiperidin-1-yl]methyl}-1H,6H,7H-pyrrolo[2,3-c]pyridin-7-one

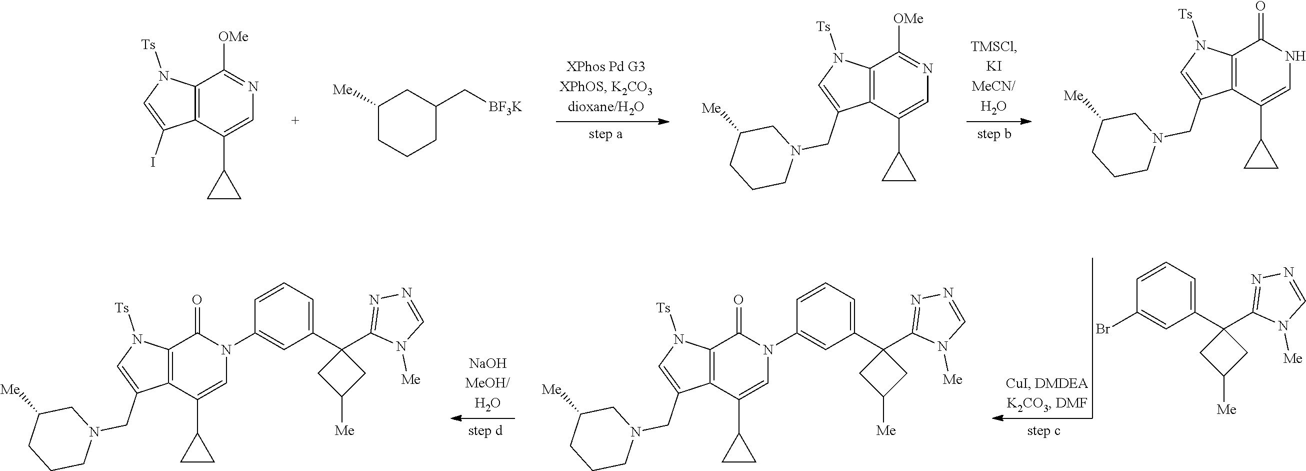

Step a: To a 40-mL vial was added 4-cyclopropyl-3-iodo-7-methoxy-1-(4-methylphenyl)sulfonylpyrrolo[2,3-c]pyridine (127 mg, 0.27 mmol, 1.0 equiv.), potassium trifluoro-{[(3S)-3-methylcyclohexyl]methyl}borate (59.4 mg, 0.27 mmol, 1.0 equiv.), XPhos Pd G3 (22.9 mg, 0.027 mmol, 10 mol %), XPhos (12.9 mg, 0.027 mmol, 10 mol %), K₂CO₃ (74.6 g, 0.54 mmol, 2.0 equiv.), dioxane (1.5 mL) and H₂O (0.50 mL). The resulting mixture was heated under N₂ at 90° C. for 3 h when LCMS showed a full consumption of the substrate iodide. After cooling to room temperature, the mixture was concentrated and the crude was purified by column chromatography (SiO₂, EtOAc in hexanes, 0 to 35%) to give the desired product.

Step b: To a mixture of the product from step a (59.4 mg, 0.14 mmol, 1.0 equiv.) in MeCN/H₂O (4.1 v/v, 2.0 mL) was added TMSCl (24.3 mg, 0.22 mmol, 1.6 equiv.) and KI (36.5 mg, 0.22 mmol, 1.6 equiv.). The resulting mixture was heated at 80° C. for 2 h when LCMS showed a completion of the demethylation. After cooling to the room temperature, the reaction mixture was concentrated to dryness. The crude residue was purified by column chromatography (SiO₂, MeOH in DCM, 0 to 10%) to give the desired product.

Step c: To a solution of the product from step b (39.2 mg, 0.089 mmol, 1.0 equiv.), 3-[1-(3-bromophenyl)-3-methylcyclobutyl]-4-methyl-1,2,4-triazole (diastereomeric mixture in 3:1 ratio, 41.0 mg, 0.13 mmol, 1.5 equiv.) in DMF (1.0 mL) was added CuI (3.4 mg, 0.018 mmol, 20 mol %), N,N′-dimethylethylenediamine (3.2 mg, 0.036 mmol, 40 mol %) and K₂CO₃ (36.9 mg, 0.27 mmol, 3.0 equiv.). The resulting mixture was heated at 110° C. for overnight. After cooling to room temperature, the reaction mixture was diluted with EtOAc and washed with water twice. The organic phase was then washed with brine, dried over Na₂SO₄ and concentrated. The crude residue was directly used in the next step.

Step d: To a solution of the crude product from step c in MeOH (2.0 mL) was added NaOH (1M in H₂O, 2.0 mL). The resulting mixture was stirred at room temperature for 3 h when LCMS showed a completed detosylation. The mixture was then diluted with EtOAc and washed with water once, and then washed with brine, dried over Na₂SO₄ and concentrated. The crude material was purified by HPLC to give the title compound as a mixture of diastereomers. ¹H NMR (400 MHz, CDCl₃) δ 9.82 (s, 1H), 8.03 (s, 0.2H), 7.97 (s, 0.8H), 7.51-7.15 (m, 5H), 6.72 (s, 1H), 4.07-3.79 (m, 2H), 3.32 (s, 0.6H), 3.26 (s, 2.4H), 3.23-2.60 (m, 7H), 2.42-2.23 (m, 2H), 2.09-1.93 (m, 1H), 1.87-1.49 (m, 4H), 1.21-1.09 (m, 3H), 1.02-0.76 (m, 6H), 0.70-0.56 (m, 2H). ESI MS [M+H]⁺ for C₃₁H₃₈N₆O, calcd 511.3, found 511.3.

### Example 16: 4-Cyclopropyl-3-(hydroxymethyl)-6-{3-[3-methyl-1-(4-methyl-4H-1,2,4-triazol-3-yl)cyclobutyl]phenyl}-1H,6H,7H-pyrrolo[2,3-c]pyridin-7-one

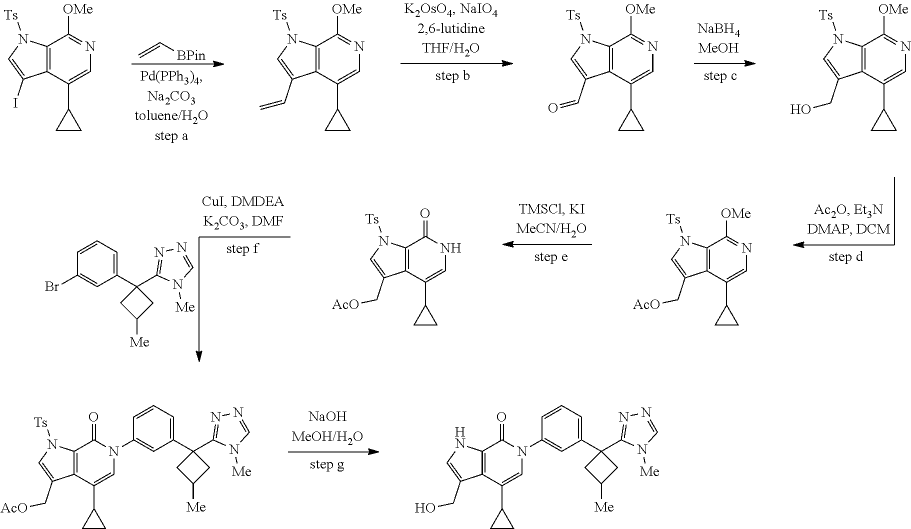

Step a: To a 40-mL vial was added 4-cyclopropyl-3-iodo-7-methoxy-1-(4-methylphenyl)sulfonylpyrrolo[2,3-c]pyridine (1.17 g, 2.5 mmol, 1.0 equiv.), vinylboronic acid pinacol ester (578 mg, 0.64 mL, 3.8 mmol, 1.5 equiv.), Pd(PPh₃)₄ (289 mg, 0.25 mmol, 10 mol %), Na₂CO₃ (1M in H₂O, 5.0 mL, 5.0 mmol, 2.0 equiv.) and toluene (10 mL). The resulting mixture was heated under N₂ at reflux for overnight. After cooling down to room temperature, the organic phase was separated, and the aqueous layer was extracted with EtOAc. The combined organic phase was dried over Na₂SO₄, concentrated and the crude residue was purified by column chromatography (SiO₂, EtOAc in hexanes, 0 to 20%) to give the vinylated product.

Step b: To a solution of the product from step a (0.81 g, 2.2 mmol, 1.0 equiv.) and 2,6-lutidine (0.47 g, 0.51 mL, 4.4 mmol, 2.0 equiv.) in THF/H₂O (1:1 v/v, 20 mL) was added K₂OsO₄·2H₂O (40 mg, 0.11 mmol, 5 mol %) and NaIO4 (1.88 g, 8.8 mmol, 4.0 equiv.). The resulting mixture was stirred at room temperature for 1.5 h. The mixture was quenched with saturated Na₂S₂O₃ (10 mL) and saturated NaHCO₃ aqueous solution (10 mL) and diluted with EtOAc. The organic layer was separated. The aqueous layer was extracted EtOAc twice. The combined organic solution was washed with brine, dried over Na₂SO₄ and concentrated. The crude residue was purified by column chromatography (SiO₂, EtOAc in hexanes, 0 to 30%) to give 4-cyclopropyl-7-methoxy-1-(4-methylphenyl)sulfonylpyrrolo[2,3-c]pyridine-3-carbaldehyde.

Step c: To a solution of the product from step b (300 mg, 0.81 mmol) in MeOH (5 mL) was added NaBH₄ (38 mg, 1.0 mmol, 1.2 equiv.). The resulting mixture was stirred at room temperature for 10 min before quenched with H₂O. The mixture was then extract with EtOAc twice. The combined organic solution was washed with brine, dried over Na₂SO₄ and concentrated. The crude product was directly used in the next step.

Step d: To a solution of the crude product from step c in DCM (4 mL) was added acetic anhydride (98 mg, 0.96 mmol), triethylamine (162 mg, 0.22 mL, 1.6 mmol) and 4-dimethylaminopyridine (10 mg, 0.081 mmol). The resulting mixture was stirred at room temperature for 30 min before quenched with H₂O. The mixture was then extract with DCM twice. The combined organic solution was washed with brine, dried over Na₂SO₄ and concentrated. The crude was directly used in the next step.

Step e: To a mixture of the crude product from step d in MeCN/H₂O (4:1 v/v, 5 mL) was added TMSCl (109 mg, 1.0 mmol) and KI (166 mg, 1.0 mmol). The resulting mixture was heated at 80° C. for 2 h when LCMS showed a completion of the demethylation. After cooling to room temperature, the reaction mixture was concentrated to dryness. The crude residue was purified by column chromatography (SiO₂, EtOAc in hexanes, 50 to 75%) to give the desired product.

Step f: To a solution of the product from step e (85.0 mg, 0.21 mmol, 1.0 equiv.), 3-[1-(3-bromophenyl)-3-methylcyclobutyl]-4-methyl-1,2,4-triazole (diastereomeric mixture in 3:1 ratio, 76.5 mg, 0.25 mmol, 1.2 equiv.) in DMF (2.0 mL) was added CuI (8.0 mg, 0.042 mmol, 20 mol %), N,N′-dimethylethylenediamine (7.4 mg, 0.084 mmol, 40 mol %) and K₂CO₃ (87.1 mg, 0.63 mmol, 3.0 equiv.). The resulting mixture was heated at 110° C. for overnight. After cooling to the room temperature, the reaction mixture was diluted with EtOAc and washed with water twice. The organic phase was then washed with brine, dried over Na₂SO₄ and concentrated. The crude was directly used in the next step.

Step g: To a solution of the crude product from step f in MeOH (2.0 mL) was added NaOH (1M in H₂O, 2.0 mL). The resulting mixture was stirred at room temperature for 3 h when LCMS showed a completed detosylation. The mixture was then diluted with EtOAc and washed with water once, and then washed with brine, dried over Na₂SO₄ and concentrated. The crude material was purified by HPLC to give the desired product as a mixture of diastereomers. ¹H NMR (400 MHz, DMSO-d₆) δ 11.91 (s, 1H), 8.36 (s, 0.2H), 8.28 (s, 0.8H), 7.55-7.12 (m, 5H), 6.72 (sz, 1H), 4.77 (s, 3H), 3.25 (s, 3H), 3.16-2.78 (m, 2H), 2.58-2.50 (m, 2H), 2.39-2.10 (m, 2H), 1.13-1.01 (m, 3H), 0.85-0.71 (m, 2H), 0.70-0.55 (m, 2H). ESI MS [M+H]⁺ for C₂₅H₂₇N₅O₂, calcd 430.2, found 430.2.

### Example 17: 4-Cyclopropyl-3-methyl-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

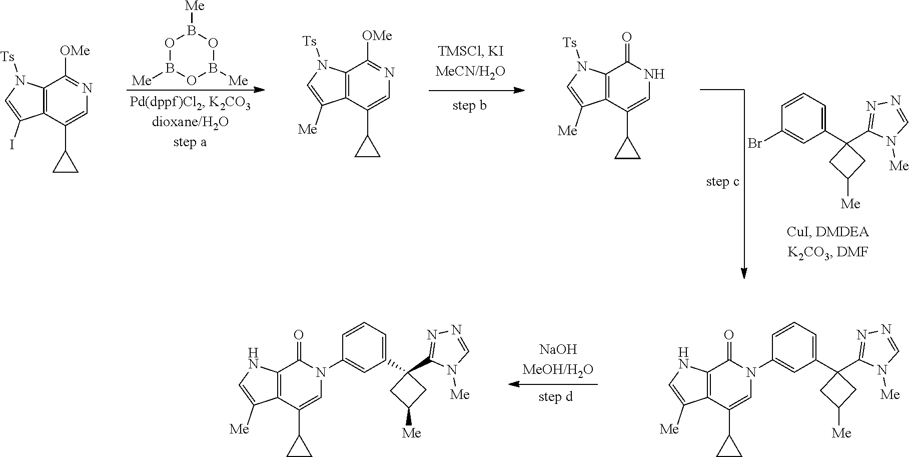

Step a: To a 40-mL vial was added 4-cyclopropyl-3-iodo-7-methoxy-1-(4-methylphenyl)sulfonylpyrrolo[2,3-c]pyridine (100 mg, 0.21 mmol, 1.0 equiv.), 2,4,6-trimethyl-1,3,5,2,4,6-trioxatriborinane (26.8 mg, 0.21 mmol, 1.0 equiv.), Pd(dppf)Cl₂ (14.6 mg, 0.021 mmol, 10 mol %), K₂CO₃ (58.0 g, 0.42 mmol, 2.0 equiv.), dioxane (1.5 mL) and H₂O (0.50 mL). The resulting mixture was heated under N₂ at 100° C. for overnight. After cooling to room temperature, the mixture was concentrated and the crude was directly used in the next step.

Step b: To a mixture of the crude product from step a in MeCN/H₂O (4:1 v/v, 2.0 mL) was added TMSCl (27.5 mg, 0.25 mmol) and KI (41.5 mg, 0.25 mmol). The resulting mixture was heated at 80° C. for 3 h when LCMS showed a completion of the demethylation. After cooling to the room temperature, the reaction mixture was concentrated to dryness. The crude residue was purified by column chromatography (SiO₂, EtOAc in hexanes, 50 to 75%) to give the desired product.

Step c: To a solution of the product from step b (42.7 mg, 0.12 mmol, 1.0 equiv.), 3-[1-(3-bromophenyl)-3-methylcyclobutyl]-4-methyl-1,2,4-triazole (diastereomeric mixture in 3:1 ratio, 57.3 mg, 0.19 mmol, 1.5 equiv.) in DMF (1.2 mL) was added CuI (4.6 mg, 0.024 mmol, 20 mol %), N,N′-dimethylethylenediamine (4.2 mg, 0.048 mmol, 40 mol %) and K₂CO₃ (46.9 mg, 0.36 mmol, 3.0 equiv.). The resulting mixture was heated at 110° C. for overnight. After cooling to room temperature, the reaction mixture was diluted with EtOAc and washed with water twice. The organic phase was then washed with brine, dried over Na₂SO₄ and concentrated. The crude residue was directly used in the next step.

Step d: To a solution of the crude product from step c in MeOH (2.0 mL) was added NaOH (1M in H₂O, 2.0 mL). The resulting mixture was stirred at room temperature for 3 h when LCMS showed a completed detosylation. The mixture was then diluted with EtOAc and washed with water once, and then washed with brine, dried over Na₂SO₄ and concentrated. The crude material was purified by WPLC to give the target molecule in 9:1 diastereomeric ratio. ¹H NMR (400 MHz, CDCl₃) δ 9.11 (s, 1H), 8.02 (s, 0.1H), 7.96 (s, 0.9H), 7.55-7.15 (m, 4H), 7.03 (s, 1H), 6.70 (s, 1H), 3.33 (s, 0.3H), 3.26 (s, 2.7H), 2.94-2.81 (m, 2H), 2.76-2.59 (m, 3H), 2.52 (s, 3H), 2.13-1.99 (m, 1H), 1.14 (d, J=5.1 Hz, 3H), 0.95-0.78 (m, 2H), 0.71-0.59 (m, 2H). ESI MS [M+H]⁺ for C₂₅H₂₇N₅O, calcd 414.2, found 414.2.

### Example 18: 4-Cyclopropyl-6-{3-[(1r,3s)-3-methyl-1-(4-methyl-4H-1,2,4-triazol-3-yl)cyclobutyl]phenyl}-3-(trifluoromethyl)-1H,6H,7H-pyrrolo[2,3-c]pyridin-7-one

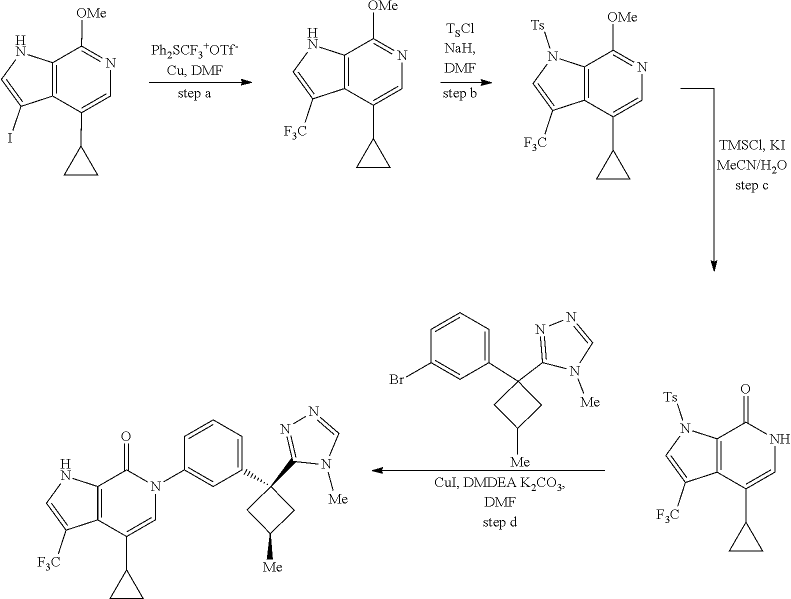

Step a: To a solution of 4-cyclopropyl-3-iodo-7-methoxy-1H-pyrrolo[2,3-c]pyridine (567 mg, 1.8 mmol, 1.0 equiv.) in DMF (6 mL) was added diphenyl(trifluoromethyl)sulfonium trifluoromethanesulfonate (2.18 g, 5.4 mmol, 3.0 equiv.) and copper (572 mg, 9.0 mmol, 5.0 equiv.). The resulting mixture was heated at 60° C. for overnight. After cooling to room temperature, the mixture was filter through Celite® and then diluted with EtOAc. The organic solution was washed with water twice and brine once, dried over Na₂SO₄ and concentrated. The crude residue was purified by column chromatography (SiO₂, EtOAc in hexanes, 0 to 25%) to give the trifluoromethylated product.

Step b: To a solution of the product from step a (262 mg, 1.0 mmol, 1.0 equiv.) in DMF (2 mL) was added NaH (60 wt % in mineral oil, 60 mg, 1.5 mmol, 1.5 equiv.) at 0° C. The resulting mixture was stirred at this temperature for 10 min before the addition of 4-toluenesulfonyl chloride (343 mg, 1.8 mmol, 1. equiv.). The reaction mixture was then raised to room temperature and stirred for overnight. The reaction was then quenched with water and diluted with EtOAc. The organic phase was separated and washed with water twice. The organic solution was then washed with brine, dried over Na₂SO₄ and concentrated. The crude residue was purified by column chromatography (SiO₂, EtOAc in hexanes, 0 to 30%) to give the tosylated product.

Step c: To a mixture of the product from step b (71.8 mg, 0.17 mmol) in MeCN/H₂O (4:1 v/v, 2 mL) was added TMSCl (38.0 mg, 0.35 mmol, 2.0 equiv.) and KI (58.1 mg, 0.35 mmol, 2.0 equiv.). The resulting mixture was heated at 80° C. for 2 h when LCMS showed a completion of the demethylation. After cooling to room temperature, the reaction mixture was concentrated to dryness. The crude residue was purified by column chromatography (SiO₂, MeOH in DCM, 0 to 10%) to give the desired product (57.1 mg, 85% yield).

Step d: To a solution of the product from step c (38.8 mg, 0.10 mmol, 1.0 equiv.), 3-[1-(3-bromophenyl)-3-methylcyclobutyl]-4-methyl-1,2,4-triazole (diastereomeric mixture in 3:1 ratio, 45.9 mg, 0.15 mmol, 1.5 equiv.) in DMF (1.0 mL) was added CuI (19.0 mg, 0.10 mmol, 1.0 equiv.), N,N′-dimethylethylenediamine (17.6 mg, 0.20 mmol, 2.0 equiv.) and K₂CO₃ (41.8 mg, 0.30 mmol, 3.0 equiv.). The resulting mixture was heated at 110° C. for 2 h when LCMS showed a full conversion to the desired product with a clean detosylation. After cooling to the room temperature, the reaction mixture was diluted with EtOAc and washed with water twice. The organic phase was then washed with brine, dried over Na₂SO₄ and concentrated. The crude residue was purified by HPLC to give the target molecule in 10:1 diastereomeric ratio. ¹H NMR (400 MHz, CDCl₃) δ 11.07 (s, 1H), 8.09 (d, J=7.8 Hz, 1H), 7.99 (s, 1H), 7.65 (s, 1H), 7.59-7.38 (m, 3H), 6.86 (s, 1H), 3.28 (s, 2.73H), 3.21 (s, 0.27H), 2.95-2.83 (m, 2H), 2.77-2.59 (m, 3H), 2.12-2.00 (m, 1H), 1.15 (d, J=5.3 Hz, 3H), 0.98-0.85 (m, 2H), 0.66-0.52 (m, 2H). ESI MS [M+H]⁺ for C₂₅H₂₄F3N₅O, calcd 468.2, found 468.2.

### Example 19 and 20: 4-Cyclopropyl-7-oxo-6-{3-[(1r,3s)-3-methyl-1-(4-methyl-4H-1,2,4-triazol-3-yl)cyclobutyl]phenyl}-1H,6H,7H-pyrrolo[2,3-c]pyridine-3-carboxylic acid and 4-Cyclopropyl-7-oxo-6-{3-[(1s,3r)-3-methyl-1-(4-methyl-4H-1,2,4-triazol-3-yl)cyclobutyl]phenyl}-1H,6H,7H-pyrrolo[2,3-c]pyridine-3-carboxylic acid

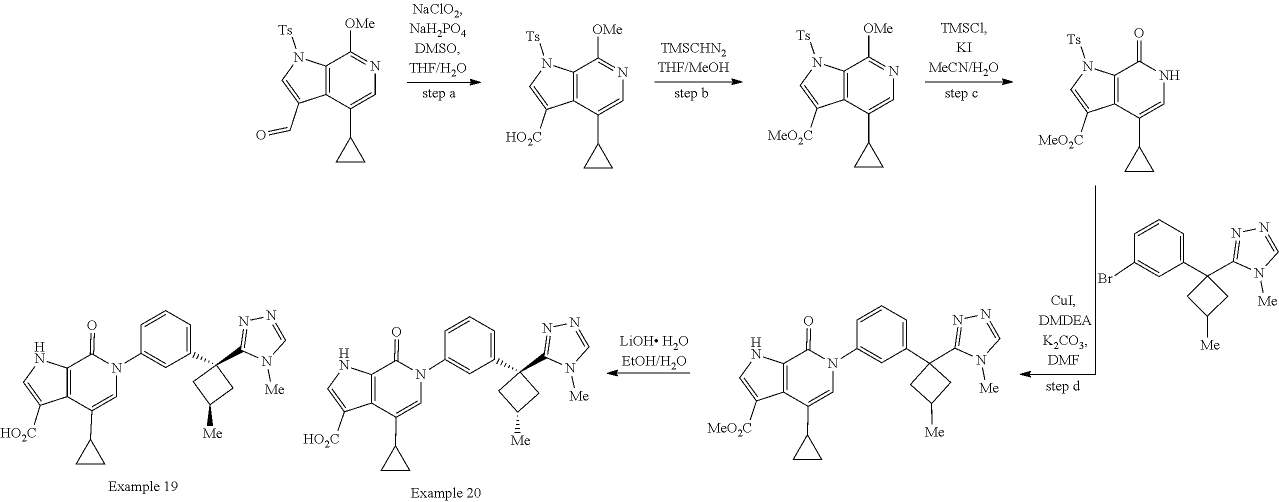

Step a: To a mixture of 4-cyclopropyl-7-methoxy-1-(4-methylphenyl)sulfonylpyrrolo[2,3-c]pyridine-3-carbaldehyde (74.8 mg, 0.20 mmol, 1.0 equiv.) in THF/H₂O (2:1 v/v, 2 mL) was added NaClO₂ (80% purity, 113 mg, 1.0 mmol, 5.0 equiv.), NaH₂PO₄ (120 mg, 1.0 mmol, 5.0 equiv.) and 5 drops of DMSO. The resulting mixture was stirred at room temperature for 1 h before quenched with saturated NaHSO₃ aqueous solution. The mixture was then acidified with 1M HCl aqueous solution to pH=1 and then extracted with EtOAc twice. The combined organic phase was then washed with brine, dried over Na₂SO₄ and concentrated. The crude was directly used in the next step.

Step b: To a solution of the crude product from step a in THF/MeOH (3:1 v/v, 4 mL) was added TMSCHN₂ (ca. 0.6 M in hexanes, 1 mL). The resulting solution was stirred at room temperature for 1 h before concentrated to afford the crude product.

Step c: To a mixture of the crude product from step b in MeCN/H₂O (4:1 v/v, 2 mL) was added TMSCl (43.5 mg, 0.40 mmol) and KI (66.4 mg, 0.40 mmol). The resulting mixture was heated at 80° C. for 2 h when LCMS showed a completion of the demethylation. After cooling to room temperature, the reaction mixture was concentrated to dryness. The crude residue was purified by column chromatography (SiO₂, MeOH in DCM, 0 to 10%) to give the desired product.

Step d: To a solution of the product from step c (75.3 mg, 0.20 mmol, 1.0 equiv.), 3-[1-(3-bromophenyl)-3-methylcyclobutyl]-4-methyl-1,2,4-triazole (diastereomeric mixture in 3:1 ratio, 91.9 mg, 0.30 mmol, 1.5 equiv.) in DMF (2.0 mL) was added CuI (38.1 mg, 0.20 mmol, 1.0 equiv.), N,N′-dimethylethylenediamine (35.3 mg, 0.40 mmol, 2.0 equiv.) and K₂CO₃ (82.9 mg, 0.60 mmol, 3.0 equiv.). The resulting mixture was heated at 110° C. for 2 h when LCMS showed the desired product with a clean detosylation. After cooling to room temperature, the reaction mixture was diluted with EtOAc and washed with water twice. The organic phase was then washed with brine, dried over Na₂SO₄ and concentrated. The crude residue was purified by column chromatography (SiO₂, MeOH in DCM, 0 to 10%) to give the desired product.

Step e: To a solution of the product from step d in EtOH/H₂O (1:1 v/v, 2 mL) was added LiOH H₂O (42.0 mg, 1.0 mmol). The resulting mixture was heated at 80° C. for 6 h when LCMS showed a completed hydrolysis. After cooling to room temperature, the reaction mixture was acidified with 1M HCl aqueous solution and extracted with EtOAc twice. The organic phase was then washed with brine, dried over Na₂SO₄ and concentrated. The crude residue was purified by HPLC to afford the title compounds.

Example 19: First eluting diastereomer (dr 10:1) ¹H NMR (400 MHz, CDCl₃) δ 12.07 (s, 1H), 8.15-7.94 (m, 2H), 7.66-7.44 (m, 3H), 7.32-7.24 (m, 1H), 6.86 (s, 1H), 3.36 (s, 0.26H), 3.30 (s, 2.74H), 2.98-2.83 (m, 2H), 2.79-2.59 (m, 4H), 1.15 (d, J=5.1 Hz, 3H), 0.95-0.80 (m, 2H), 0.64-0.43 (m, 2H). ESI MS [M+H]⁻ for C₂₅H₂₅N₅O₃, calcd 444.2, found 444.2.

Example 20: Second eluting diastereomer (dr 2:1)¹H NMR (400 MHz, CDCl₃) δ 11.75 (s, 1H), 8.18-7.99 (m, 2H), 7.74-7.43 (m, 3H), 7.37-7.31 (m, 1H), 6.89 (s, 1H), 3.38 (s, 2H), 3.33 (s, 1H), 3.29-2.84 (m, 2H), 2.80-2.53 (m, 3H), 2.44-2.31 (m, 1H), 1.17 (d, J=6.3 Hz, 3H), 0.98-0.86 (m, 2H), 0.69-0.55 (m, 2H). ESI MS [M+H]⁺ for C₂₅H₂₅N₅O₃, calcd 444.2, found 444.2.

### Example 21 and 22: 3-Acetyl-4-cyclopropyl-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one and 3-Acetyl-4-cyclopropyl-6-{3-[(1s,3r)-3-methyl-1-(4-methyl-4H-1,2,4-triazol-3-yl)cyclobutyl]phenyl}-1H,6H,7H-pyrrolo[2,3-c]pyridin-7-one

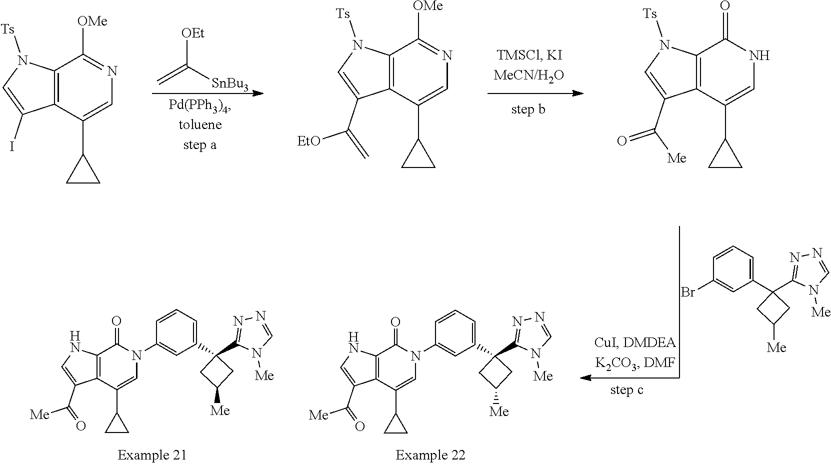

Step a: To a 40-mL vial was added 4-cyclopropyl-3-iodo-7-methoxy-1-(4-methylphenyl)sulfonylpyrrolo[2,3-c]pyridine (328 mg, 0.70 mmol, 1.0 equiv.), tributyl(1-ethoxyvinyl)tin (278 mg, 0.26 mL, 0.77 mmol, 1.1 equiv.), Pd(PPh₃)₄ (80.9 mg, 0.070 mmol, 10 mol %) and toluene (3.0 mL). The resulting mixture was heated under N₂ at reflux for overnight. After cooling to room temperature, the mixture was concentrated, and the crude was purified by column chromatography (SiO₂, EtOAc in hexanes, 0 to 15%) to give the desired product.

Step b: To a mixture of the crude product from step a (267 mg, 0.65 mmol, 1.0 equiv.) in MeCN/H₂O (4:1 v/v, 4 mL) was added TMSCl (140 mg, 0.16 mL, 1.3 mmol, 2.0 equiv.) and KI (216 mg, 1.3 mmol, 2.0 equiv.). The resulting mixture was heated at 80° C. for 2 h when LCMS showed a completion of the demethylation. After cooling to room temperature, the reaction mixture was concentrated to dryness. The crude residue was purified by column chromatography (SiO₂, MeOH in DCM, 0 to 10%) to give the desired product.

Step c: To a solution of the product from step b (100 mg, 0.27 mmol, 1.0 equiv.), 3-[1-(3-bromophenyl)-3-methylcyclobutyl]-4-methyl-1,2,4-triazole (diastereomeric mixture in 3:1 ratio, 122 mg, 0.40 mmol, 1.5 equiv.) in DMF (2.7 mL) was added CuI (51.4 mg, 0.27 mmol, 1.0 equiv.), N,N′-dimethylethylenediamine (47.6 mg, 0.54 mmol, 2.0 equiv.) and K₂CO₃ (113 mg, 0.81 mmol, 3.0 equiv.). The resulting mixture was heated at 110° C. for 4 h when LCMS showed the desired product with a clean detosylation. After cooling to the room temperature, the reaction mixture was diluted with EtOAc and washed with water twice. The organic phase was then washed with brine, dried over Na₂SO₄ and concentrated. The crude residue was purified by column chromatography (SiO₂, MeOH in DCM, 0 to 10%) and further purified by HPLC to afford the title compounds.

Example 21: First eluting diastereomer (dr 14:1) ¹H NMR (400 MHz, CDCl₃) δ 11.82 (s, 1H), 8.00 (s, 1H), 7.92 (s, 1H), 7.58-7.41 (m, 3H), 7.32-7.23 (m, 1H), 6.83 (s, 1H), 3.33 (s, 0.19H), 3.28 (s, 2.81H), 2.98-2.82 (m, 2H), 2.81-2.63 (m, 4H), 2.60 (s, 3H), 1.15 (d, J=5.1 Hz, 3H), 0.91-0.80 (m, 2H), 0.54-0.42 (m, 2H). ESI MS [M+H]⁺ for C₂₆H₂₇N₅O₂, calcd 442.2, found 442.2.

Example 22: Second eluting diastereomer (dr 2:1)¹H NMR (400 MHz, CDCl₃) δ 11.35 (s, 1H), 8.06 (s, 1H), 7.91 (s, 1H), 7.56-7.19 (m, 4H), 6.83 (s, 1H), 3.34 (s, 2H), 3.28 (s, 1H), 3.25-2.81 (m, 2H), 2.77-2.66 (m, 2H), 2.60 (s, 4H), 2.40-2.26 (m, 1H), 1.14 (d, J=6.3 Hz, 3H), 0.92-0.82 (m, 2H), 0.61-0.43 (m, 2H). ESI MS [M+H]⁺ for C₂₆H₂₇N₅O₂, calcd 442.2, found 442.2.

### Example 23 and 24: 4-Cyclopropyl-3-(1-hydroxyethyl)-6-{3-[(1R,3S)-3-methyl-1-(4-methyl-4H-1,2,4-triazol-3-yl)cyclobutyl]phenyl}-1H,6H,7H-pyrrolo[2,3-c]pyridin-7-one and 4-Cyclopropyl-3-(1-hydroxyethyl)-6-{3-[(1S,3R)-3-methyl-1-(4-methyl-4H-1,2,4-triazol-3-yl)cyclobutyl]phenyl}-1H,6H,7H-pyrrolo[2,3-c]pyridin-7-one

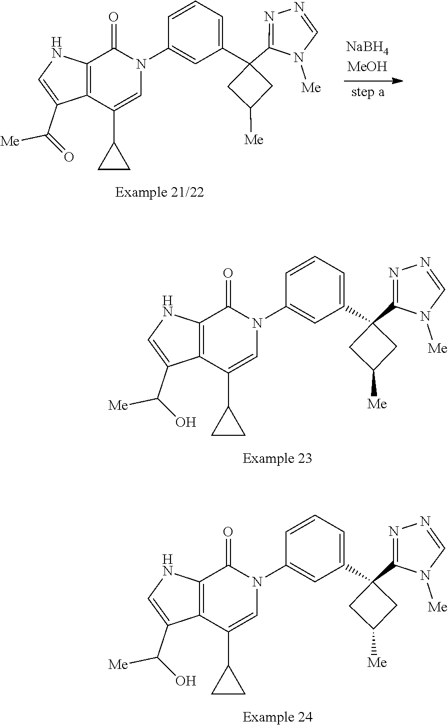 [SMILES: CC(=O)c1c[nH]c2c(=O)n(-c3cccc(C4(c5nncn5C)CC(C)C4)c3)cc(C3CC3)c12 ; CC(O)c1c[nH]c2c(=O)n(-c3cccc([C@]4(c5nncn5C)C[C@@H](C)C4)c3)cc(C3CC3)c12 ; CC(O)c1c[nH]c2c(=O)n(-c3cccc([C@]4(c5nncn5C)C[C@H](C)C4)c3)cc(C3CC3)c12]

Step a: To a solution of the mixture of Example 21 and 22 (ca. 3:1 ratio, 27.0 mg, 0.061 mmol, 1.0 equiv.) in MeOH (1.0 mL) was added NaBH₄ (6.8 mg, 0.18 mmol, 3.0 equiv.). The resulting mixture was stirred at room temperature for overnight before quenched with water. The mixture was then extracted with EtOAc twice. The organic phase was then washed with brine, dried over Na₂SO₄ and concentrated. The crude residue was purified by HPLC to afford the title compounds.

Example 23: First eluting diastereomer (dr 10:1) ¹H NMR (400 MHz, CDCl₃) δ 10.67 (s, 1H), 7.96 (s, 1H), 7.54-7.21 (m, 5H), 6.74 (s, 1H), 5.60 (q, J=6.4 Hz, 1H), 3.33 (s, 0.27H), 3.27 (s, 2.73H), 3.00-2.81 (m, 2H), 2.77-2.60 (m, 3H), 2.37-2.13 (m, 2H), 1.68 (d, J=6.4 Hz, 3H), 1.14 (d, J=5.0 Hz, 3H), 0.98-0.85 (m, 2H), 0.77-0.66 (m, 1H), 0.67-0.54 (m, 1H). ESI MS [M+H]⁺ for C₂₆H₂₉N₅O₂, calcd 444.2, found 444.2.

Example 24: Second eluting diastereomer (dr 3:1) ¹H NMR (400 MHz, CDCl₃) δ 10.43 (s, 1H), 8.03 (s, 0.73H), 7.97 (s, 0.27H), 7.53-7.16 (m, 5H), 6.75 (s, 1H), 5.60 (q, J=6.5 Hz, 1H), 3.34 (s, 2H), 3.28 (s, 1H), 3.24-2.80 (m, 2H), 2.77-2.52 (m, 2H), 2.41-2.17 (m, 3H), 1.68 (d, J=6.4 Hz, 3H), 1.13 (d, J=6.3 Hz, 3H), 1.00-0.86 (m, 2H), 0.80-0.68 (m, 1H), 0.66-0.55 (m, 1H). ESI MS [M+H]⁺ for C₂₆H₂₉N₅O₂, calcd 444.2, found 444.3.

### Example 25 and 26: 4-Cyclopropyl-3-methanesulfonyl-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one and 4-Cyclopropyl-3-methanesulfonyl-6-{3-[(1s,3r)-3-methyl-1-(4-methyl-4H-1,2,4-triazol-3-yl)cyclobutyl]phenyl}-1H,6H,7H-pyrrolo[2,3-c]pyridin-7-one

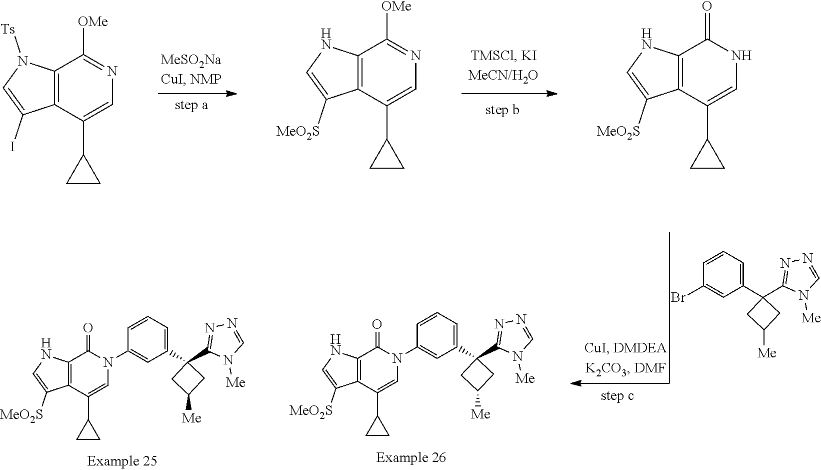 [SMILES: COc1ncc(C2CC2)c2c(I)cn(S(=O)(=O)c3ccc(C)cc3)c12 ; COc1ncc(C2CC2)c2c(S(C)(=O)=O)c[nH]c12 ; CS(=O)(=O)c1c[nH]c2c(=O)[nH]cc(C3CC3)c12 ; Cn1cnnc1[C@]1(c2cccc(-n3cc(C4CC4)c4c(S(C)(=O)=O)c[nH]c4c3=O)c2)C[C@H](C)C1 ; Cn1cnnc1[C@]1(c2cccc(-n3cc(C4CC4)c4c(S(C)(=O)=O)c[nH]c4c3=O)c2)C[C@@H](C)C1 ; CC1CC(c2cccc(Br)c2)(c2nncn2C)C1]

Step a: To a solution of 4-cyclopropyl-3-iodo-7-methoxy-1-(4-methylphenyl)sulfonylpyrrolo[2,3-c]pyridine (100 mg, 0.21 mmol, 1.0 equiv.) in NMP (2.0 mL) was added sodium methanesulfinate (85% purity, 132 mg, 1.1 mmol, 5.0 equiv.) and CuI (209 mg, 1.1 mmol, 5.0 equiv.). The resulting mixture was heated at 110° C. for overnight. After cooling to room temperature, the reaction mixture was diluted with EtOAc and washed with water twice. The organic phase was then washed with brine, dried over Na₂SO₄ and concentrated. The crude was purified by column chromatography (SiO₂, MeOH in DCM, 0 to 10%) to give the desired product.

Step b: To a mixture of the product from step a (148 mg, 0.22 mmol, 1.0 equiv.) in MeCN/H₂O (4:1 v/v, 2 mL) was added TMSCl (35.8 mg, 0.33 mmol, 1.5 equiv.) and KI (54.8 mg, 0.33 mmol, 1.5 equiv.). The resulting mixture was heated at 80° C. for 2 h when LCMS showed a completion of the demethylation. After cooling to room temperature, the reaction mixture was concentrated to dryness. The crude residue was purified by column chromatography (SiO₂, MeOH in DCM, 0 to 20%) to give the desired product.

Step c: To a solution of the product from step b (41.9 mg, 0.17 mmol, 1.0 equiv.), 3-[1-(3-bromophenyl)-3-methylcyclobutyl]-4-methyl-1,2,4-triazole (diastereomeric mixture in 3:1 ratio, 76.3 mg, 0.25 mmol, 1.5 equiv.) in DMF (1.7 mL) was added CuI (32.4 mg, 0.17 mmol, 1.0 equiv.), N,N′-dimethylethylenediamine (30.9 mg, 0.17 mmol, 2.0 equiv.) and K₂CO₃ (72.4 mg, 0.52 mmol, 3.0 equiv.). The resulting mixture was heated at 110° C. for 2 h when LCMS showed the desired product with a clean detosylation. After cooling to the room temperature, the reaction mixture was diluted with EtOAc and washed with water twice. The organic phase was then washed with brine, dried over Na₂SO₄ and concentrated. The crude residue was purified by column chromatography (SiO₂, MeOH in DCM, 0 to 20%) and further purified by HPLC to afford the title compounds.

Example 25: First eluting diastereomer (dr>20:1) ¹H NMR (400 MHz, CDCl₃) δ 12.65 (s, 1H), 8.07 (s, 1H), 7.81 (s, 1H), 7.63-7.42 (m, 3H), 7.37 (s, 1H), 6.81 (s, 1H), 3.34 (s, 3H), 3.31 (s, 3H), 2.94-2.82 (m, 2H), 2.77-2.57 (m, 4H), 1.16 (d, J=5.1 Hz, 3H), 1.03-0.89 (m, 2H), 0.76-0.63 (m, 2H). ESI MS [M+H]⁺ for C₂₅H₂₇N₅O₃S, calcd 478.2, found 478.2.

Example 26: Second eluting diastereomer (dr 3:1)¹H NMR (400 MHz, CDCl₃) δ 11.92 (s, 1H), 8.13-8.06 (m, 1H), 7.94-7.84 (m, 1H), 7.57-7.15 (m, 4H), 6.82 (s, 1H), 3.35 (s, 5H), 3.30 (s, 1H), 3.26-2.83 (m, 2H), 2.79-2.50 (m, 3H), 2.32 (t, J=10.3 Hz, 1H), 1.15 (d, J=6.3 Hz, 3H), 1.06-0.91 (m, 2H), 0.77-0.56 (m, 2H). ESI MS [M+H]⁺ for C₂₅H₂₇N₅O₃S, calcd 478.2, found 478.2.

Example 27 and 28: 7-Oxo-6-{3-[(1r,3s)-3-methyl-1-(4-methyl-4H-1,2,4-triazol-3-yl)cyclobutyl]phenyl}-1H,6H,7H-pyrrolo[2,3-c]pyridine-3-carbonitrile and 7-Oxo-6-{3-[(1s,3r)-3-methyl-1-(4-methyl-4H-1,2,4-triazol-3-yl)cyclobutyl]phenyl}-1H,6H,7H-pyrrolo[2,3-c]pyridine-3-carbonitrile

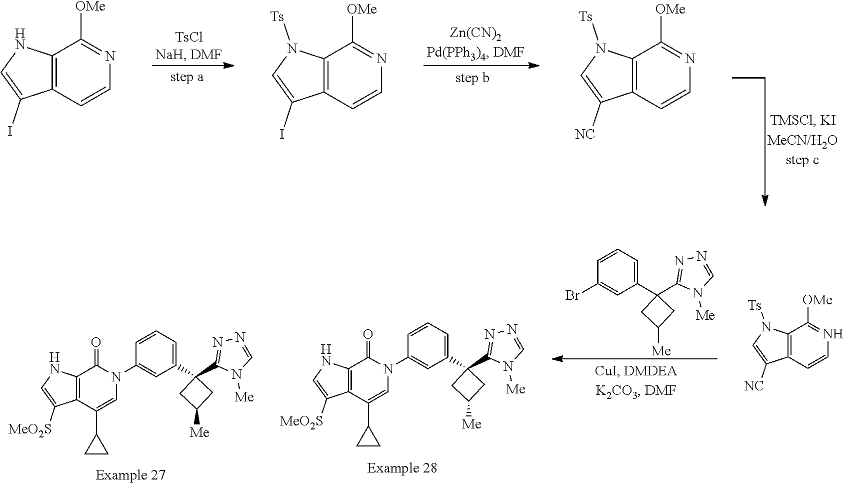

Step a: To a solution of 3-iodo-7-methoxy-1H-pyrrolo[2,3-c]pyridine (500 mg, 1.8 mmol, 1.0 equiv.) in DMF (3.6 mL) was added NaH (60 wt % in mineral oil, 80 mg, 2.0 mmol, 1.1 equiv.) at 0° C. The resulting mixture was stirred at this temperature for 10 min before the addition of 4-toluenesulfonyl chloride (381 mg, 2.0 mmol, 1.1 equiv.). The reaction mixture was then raised to room temperature and stirred for overnight. The reaction was then quenched with water and diluted with EtOAc. The organic phase was separated and washed with water twice. The organic solution was then washed with brine, dried over Na₂SO₄ and concentrated. The crude residue was purified by column chromatography (SiO₂, EtOAc in hexanes, 0 to 15%) to give the tosylated product.

Step b: To a solution of the product from step a (150 mg, 0.35 mmol, 1.0 equiv.) in DMF (2.0 mL) was added Zn(CN)₂ (62 mg, 0.53 mmol, 1.5 equiv.) and Pd(PPh₃)₄ (40 mg, 0.035 mmol, 10 mol %). The resulting mixture was heated under N₂ at 100° C. for 1.5 h when LCMS showed a completion of the cyanation. After cooling to room temperature, the reaction mixture was diluted with EtOAc and washed with water twice. The organic phase was then washed with brine, dried over Na₂SO₄ and concentrated. The crude was directly used in the next step.

Step c: To a mixture of the crude product from step b in MeCN/H₂O (4:1 v/v, 2 mL) was added TMSCl (60 mg, 0.55 mmol) and KI (91 mg, 0.55 mmol). The resulting mixture was heated at 80° C. for 2 h when LCMS showed a completion of the demethylation. After cooling to room temperature, the reaction mixture was concentrated to dryness. The crude residue was purified by column chromatography (SiO₂, MeOH in DCM, 0 to 10%) to give the desired product.

Step d: To a solution of the product from step c (63.0 mg, 0.20 mmol, 1.0 equiv.), 3-[1-(3-bromophenyl)-3-methylcyclobutyl]-4-methyl-1,2,4-triazole (diastereomeric mixture in 3:1 ratio, 61.2 mg, 0.20 mmol, 1.0 equiv.) in DMF (2.0 mL) was added CuI (38.1 mg, 0.20 mmol, 1.0 equiv.), N,N′-dimethylethylenediamine (35.3 mg, 0.40 mmol, 2.0 equiv.) and K₂CO₃ (82.9 mg, 0.60 mmol, 3.0 equiv.). The resulting mixture was heated at 110° C. for 2 h when LCMS showed a full conversion to the desired product with a clean detosylation. After cooling to the room temperature, the reaction mixture was diluted with EtOAc and washed with water twice. The organic phase was then washed with brine, dried over Na₂SO₄ and concentrated. The crude residue was purified by column chromatography (SiO₂, MeOH in DCM, 0 to 10%) and further purified by HPLC to afford the title compounds.

Example 27: First eluting diastereomer (dr 5:1) ¹H NMR (400 MHz, CD₃OD) δ 8.37 (s, 0.16H), 8.30 (s, 0.84H), 7.99 (s, 1H), 7.63-7.25 (m, 5H), 6.79 (d, J=7.1 Hz, 1H), 3.34 (s, 3H), 2.96 (dt, J=7.8, 4.3 Hz, 2H), 2.76-2.50 (m, 3H), 1.14 (d, J=6.0 Hz, 3H). ESI MS [M+H]⁺ for C₂₂H₂ON₆O, calcd 385.2, found 385.2.

Example 28: Second eluting diastereomer (dr 2:1)¹H NMR (400 MHz, CD₃OD) δ 8.37 (s, 0.66H), 8.30 (s, 0.34H), 8.00 (s, 1H), 7.60-7.22 (m, 5H), 6.78 (dd, J=7.2, 1.4 Hz, 1H), 3.40-3.32 (m, 3H), 3.20-2.86 (m, 2H), 2.70-2.29 (m, 3H), 1.14 (d, J=6.2 Hz, 3H). ESI MS [M+H]⁻ for C₂₂H₂ON₆O, calcd 385.2, found 385.2.

### Example 29: 4-methyl-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

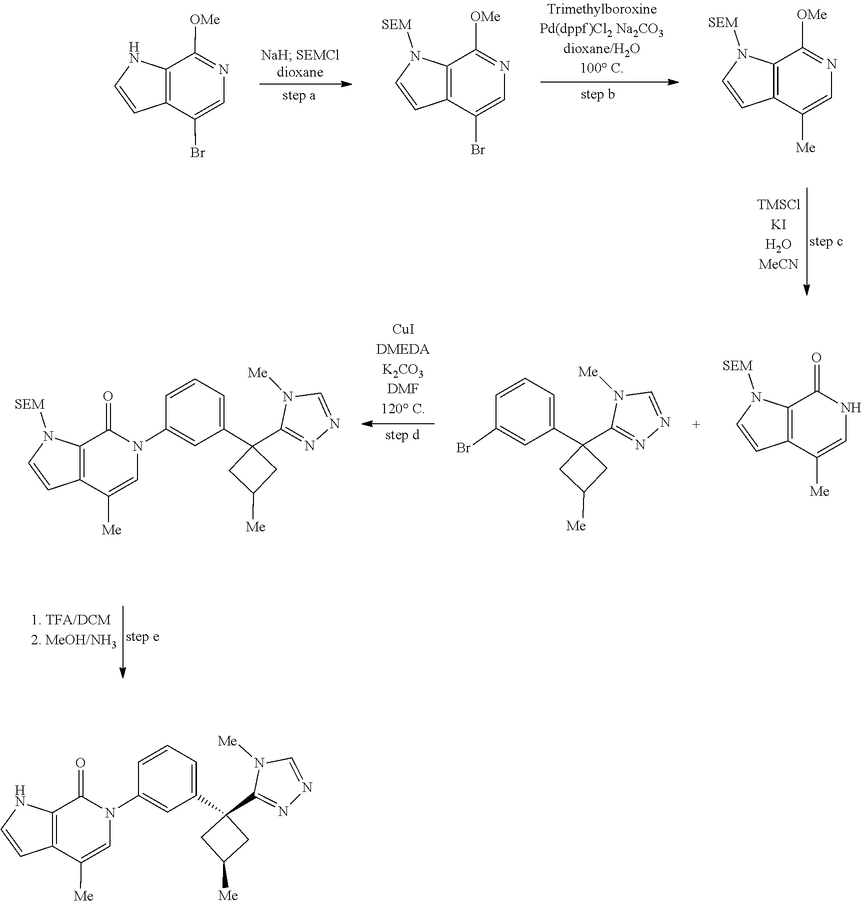

Step a: The desired product was prepared in a similar manner to Example 7, step a.

Step b: To the product of step a (357 mg, 1.0 mmol) was added trimethylboroxine (126 mg, 1.0 mmol, 1 equiv.), Na₂CO₃ (212 mg, 2.0 mmol, 2 equiv.), dioxane (4 mL), and water (1 mL). The mixture was sparged with N₂ for 10 minutes, after which Pd(dppf)Cl₂ (73 mg, 0.1 mmol, 10%) was added and the mixture was heated to 100° C. for 16 hours. The reaction mixture was partitioned between EtOAc and water, the aqueous was extracted three times, and the combined organics were dried over Na₂SO₄ and concentrated. Flash column chromatography (SiO₂, 0 to 50% EtOAc/hexanes gradient) gave rise to 2-[(7-methoxy-4-methylpyrrolo[2,3-c]pyridin-1-yl)methoxy]ethyl-trimethylsilane.

Step c: To a solution of 2-[(7-methoxy-4-methylpyrrolo[2,3-c]pyridin-1-yl)methoxy]ethyl-trimethylsilane (160 mg, 0.55 mmol) in MeCN (5.5 mL) was added KI (146 mg, 0.88 mmol, 1.6 equiv.), H₂O (100 DL), and TMSCl (112 QL, 0.88 mmol, 1.6 equiv.) and the mixture was stirred at room temperature for 16 hours. The reaction mixture was partitioned between EtOAc and water, the aqueous was extracted three times, and the combined organics were dried over Na₂SO₄ and concentrated. Flash column chromatography (SiO₂, 0 to 10% MeOH/DCM gradient) followed by a second column chromatography (SiO₂, 0 to 100% EtOAc/DCM) gave rise to 4-methyl-1-(2-trimethylsilylethoxymethyl)-6H-pyrrolo[2,3-c]pyridin-7-one.

Step d: The desired product was prepared in a similar manner to Example 1, step f.

Step e: The desired products were prepared in a similar manner to Example 7, step e. (dr 10:1)¹H NMR (400 MHz, CDCl₃) δ 9.83 (br s, 1H), 7.96 (s, 1H), 7.50-7.44 (m, 2H), 7.33 (dddd, J=17.3, 7.8, 2.0, 1.1 Hz, 2H), 7.27 (m, 1H), 6.80 (q, J=1.2 Hz, 1H), 6.44 (dd, J=2.9, 2.2 Hz, 1H), 3.26 (s, 3H), 2.94-2.82 (m, 2H), 2.75-2.63 (m, 3H), 2.28 (d, J=1.2 Hz, 3H), 1.14 (d, J=5.7 Hz, 3H). ESI MS [M+H]⁺ for C₂₂H₂₄N₅O, calcd 374.2, found 374.2.

### Example 30 and 31: 6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one and 6-{3-[(1s,3r)-3-methyl-1-(4-methyl-4H-1,2,4-triazol-3-yl)cyclobutyl]phenyl}-1H,6H,7H-pyrrolo[2,3-c]pyridin-7-one

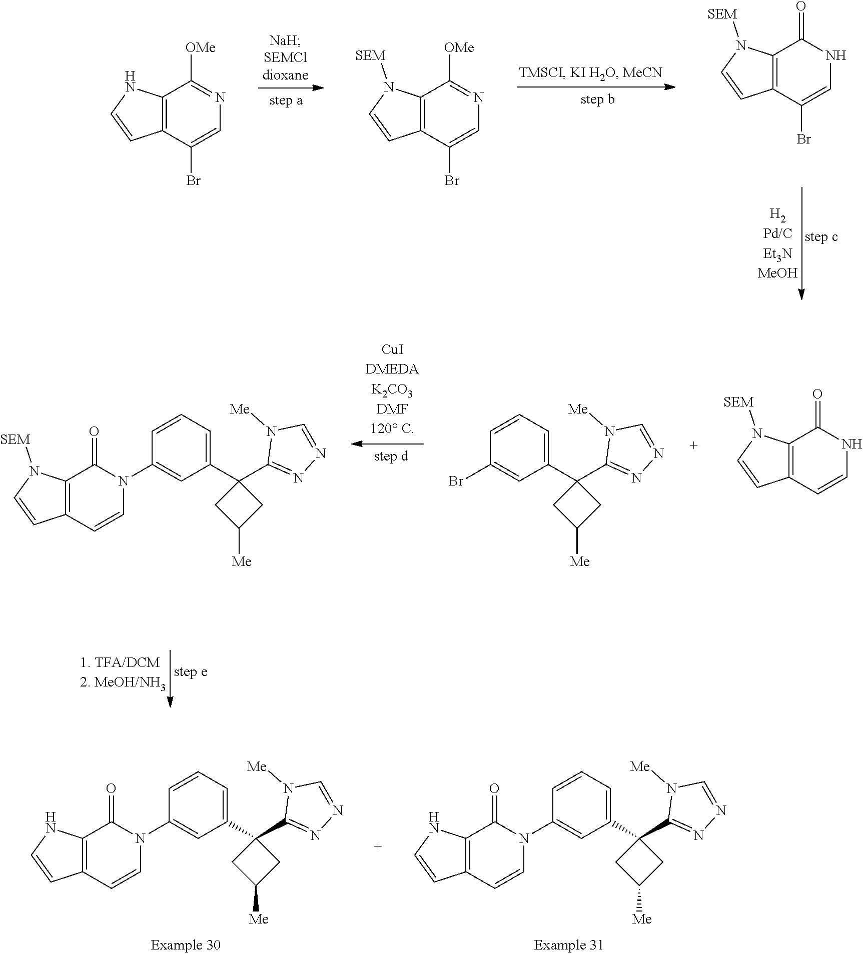

Step a: The desired product was prepared in a similar manner Example 7, step a.

Step b: The desired product was prepared in a similar manner to Example 29, step c.

Step c: 4-bromo-1-(2-trimethylsilylethoxymethyl)-6H-pyrrolo[2,3-c]pyridin-7-one (293 mg, 0.85 mmol) was dissolved in MeOH (20 mL), Pd/C (30 mg, 30% wt.), and Et₃N (120 mL, 0.85 mmol, 1 equiv.) were added and mixture was shaken in a Parr shaker apparatus under an atmosphere of H₂ (30 psi) for 16 hours. Upon completion, the reaction mixture was filtered through Celite®, washed with EtOAc, and concentrated. The crude material was purified by flash column chromatography (SiO₂, 0 to 10% MeOH/DCM) to yield 1-(2-trimethylsilylethoxymethyl)-6H-pyrrolo[2,3-c]pyridin-7-one.

Step d: The desired product was prepared in a similar manner to Example 1, step f.

Step e: The desired products were prepared in a similar manner to Example 7, step e.

Example 30: First eluting diastereomer (10:1 d.r.) ¹H NMR (400 MHz, CDCl₃δ 9.81 (s, 1H), 7.97 (d, J=0.4 Hz, 1H), 7.52-7.40 (m, 2H), 7.37 (ddd, J=7.9, 1.9, 1.1 Hz, 1H), 7.37-7.18 (m, 2H), 7.02 (d, J=7.2 Hz, 1H), 6.64 (dd, J=7.1, 0.7 Hz, 1H), 6.44 (dd, J=2.8, 2.2 Hz, 1H), 3.26 (s, 3H), 2.88 (td, J=7.3, 6.4, 3.5 Hz, 2H), 2.74-2.60 (m, 3H), 1.17-1.09 (m, 3H). ESI MS [M+H]⁺ for C₂₁H₂₂N₅O, calcd 360.2, found 360.2.

Example 31: Second eluting diastereomer (2.3:1 d.r.) ¹H NMR (400 MHz, CDCl₃) δ 9.56 (s, 1H), 8.03 (s, 0.7H), 7.97 (s, 0.3H), 7.52-7.43 (m, 1H), 7.47-7.33 (m, 1H), 7.35-7.24 (m, 2H), 7.26-7.19 (m, 1H), 7.01 (dd, J=7.2, 4.3 Hz, 1H), 6.64 (dd, J=7.1, 0.6 Hz, 1H), 6.45 (dd, J=2.9, 2.2 Hz, 1H), 3.33 (s, 2.1H), 3.27 (s, 0.9H), 3.23-3.13 (m, 1.3H), 2.88 (d, J=4.0 Hz, 0.8H), 2.68 (d, J=7.5 Hz, 0.9H), 2.64-2.53 (m, 0.7H), 2.31 (td, J=9.4, 2.6 Hz, 1.3H), 1.13 (m, 3H). ESI MS [M+H]⁺ for C₂₁H₂₂N₅O, calcd 360.2, found 360.2.

### Example 32: 4-cyclopropyl-6-{3-[3-methyl-1-(4-methyl-4H-1,2,4-triazol-3-yl)cyclobutyl]phenyl}-2-(1-methyl-1H-pyrazol-4-yl)-1H,6H,7H-pyrrolo[2,3-c]pyridin-7-one

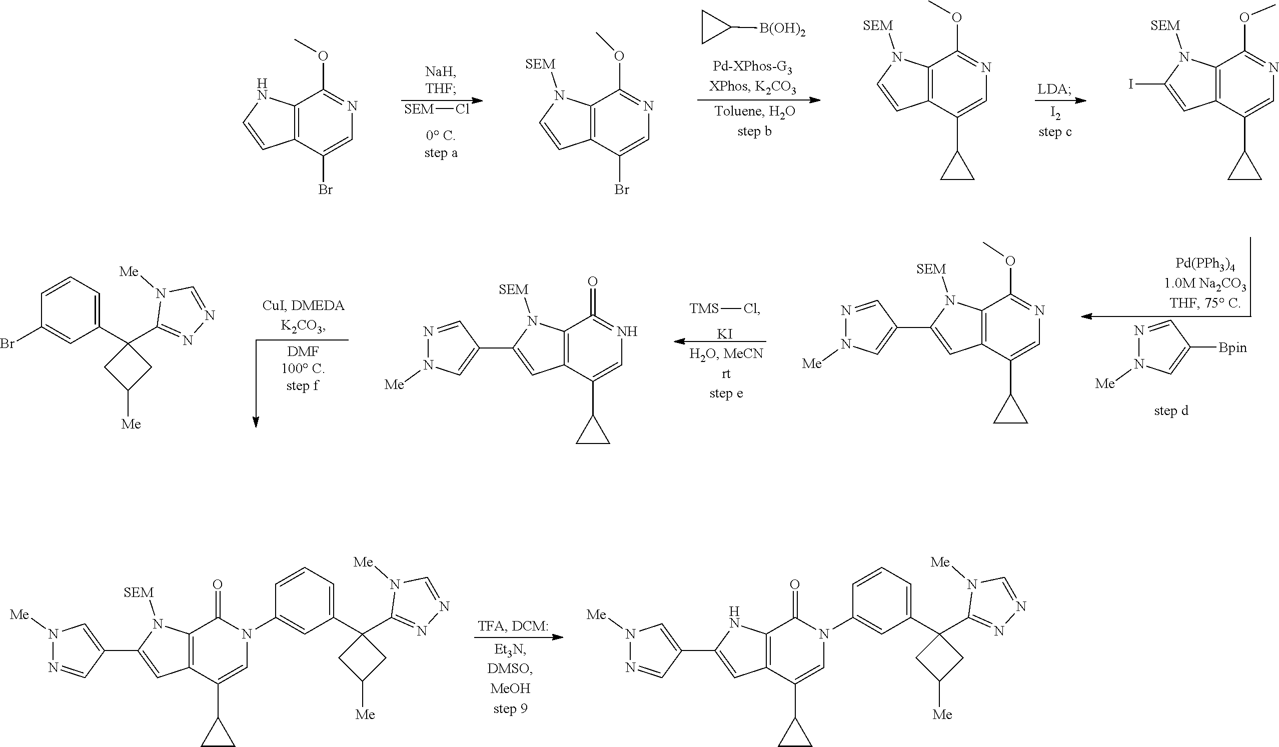

Step a: To a solution of 4-Bromo-7-methoxy-1H-pyrrolo[2,3-c]pyridine (2.0 g, 8.81 mmol) in THF (30 mL) at 0° C. was added NaH (60 wt % dispersion in oil, 405 mg, 10.13 mmol) in four portions over five minutes. The reaction was stirred at 0° C. for 30 minutes after the last portion was added, then SEM-Cl (1.79 mL, 10.13 mmol) was added. The reaction was stirred for two hours are 0° C. then slowly warmed to 10° C. at which point the reaction was complete. The reaction was quenched with water then diluted with EtOAc and water. The organics were washed with brine, dried over MgSO₄ and concentrated under reduced pressure. Column chromatograph (SiO₂, 0 to 20% EtOAc/Hex) afforded the desired product.

Step b: A suspension of the product from step a (1.75 g, 4.89 mmol), cyclopropylboronic acid (504 mg, 5.87 mmol), K₂CO₃ (2.03 g, 14.7 mmol), Xphos Pd G3 (203 mg, 0.24 mmol), XPhos (114 mg, 0.24 mmol) in degassed toluene (18.4 mL) and water (6.1 mL) was heated to 90° C. overnight. After cooling to room temperature, the reaction was diluted with EtOAc and washed with water and brine. The organics were dried with MgSO₄ and concentrated under reduced pressure. Column chromatograph (SiO₂, 0 to 15% EtOAc/DCM) afforded the desired product.

Step c: To a solution of the product from b (1.09 g, 3.42 mmol) in THF (17 mL) at −78° C. was added LDA (2.0M, 2.22 mL, 4.45 mmol). The mixture was stirred for 1 hour then iodine (1.74 g, 6.84 mmol) was added in a single portion. The mixture was stirred at −78° C. for four hours then quenched with saturated sodium thiosulfate. After warming to room temperature, the reaction was partitioned between ethyl acetate and water. The organics were washed with brine, dried over MgSO₄ and concentrated under reduced pressure. Column chromatograph (SiO₂, 0 to 30% EtOAc/Hex) afforded the desired product.

Step d: To a flask charged with the product from step c (150 mg, 0.34 mmol), 1-Methyl-4-(4,4,5,5-tetramethyl-1,3,2-dioxaborolan-2-yl)-1H-pyrazole (92 mg, 0.44 mmol, Pd(PPh₃)₄ (40 mg, 0.034 mmol) was added degassed THF (3.4 mL) and Na₂CO₃ (1.0M/H₂O, 1.4 mL). The mixture was heated to 80° C. overnight. After cooling to room temperature, the reaction was diluted with EtOAc and washed with water and brine. The organics were dried with MgSO₄ and concentrated under reduced pressure. Column chromatograph (SiO₂, 0 to 15% EtOAc/DCM) afforded the desired product.

Step e: To a flask charged with the product from step d (80 mg, 0.20 mmol) and KI (53 mg, 0.32 mmol) was added MeCN (2.0 mL) and water (1 drop) followed by TMS-Cl (0.04 mL, 0.32 mmol). After stirring overnight, the reaction was diluted with EtOAc and washed with water and brine. The organics were dried with MgSO₄ and concentrated under reduced pressure. Column chromatograph (SiO₂, 0 to 40% EtOAc/DCM) afforded the desired product.

Step f: A flask charged with the product from step e (62 mg, 0.16 mmol), 3-[1-(3-bromophenyl)-3-methylcyclobutyl]-4-methyl-1,2,4-triazole (59 mg, 0.19 mmol), CuI (6 mg, 0.032 mmol), and K₂CO₃ (66 mg, 0.48 mmol) was evacuated a backfilled with nitrogen three times then DMEDA (0.014 mL, 0.13 mmol) in degassed DMF (1.6 mL) was added. The mixture was heated to 100° C. overnight under nitrogen. After cooling to room temperature, the reaction was diluted with water. The sticky precipitate was isolated via decantation, dried under vacuum, the used without further purification.

Step h: To a solution of the product the f (110 mg, 0.18 mmol) in DCM (0.9 mL) was added TFA (0.9 mL). The mixture was stirred at room temperature to 30 minutes, then diluted with toluene and concentrated to dryness. The residue was reconstituted in DMSO (1 mL) and methanol (0.5 mL) then Et₃N (0.075 mL) was added, and the reaction stirred overnight. This mixture was purified directly by preparative HPLC (C18, gradient MeCN/H₂O) to afford the title compound as 3:1 mixture of diastereomers. ¹H NMR (400 MHz, CDCl₃) δ 10.88-10.67 (m, 1H), 7.95 (s, 0.3H), 7.88 (s, 0.7H), 7.73 (s, 1H), 7.68 (s, 1H), 7.57 (s, 1H), 7.53-7.44 (m, 1H), 7.40 (d, J=7.6 Hz, 1H), 7.32 (d, J=7.6 Hz, 1H), 3.85-3.68 (m, 3H), 3.18 (s, 2H), 2.92-2.78 (m, 2H), 2.71-2.47 (m, 3H), 2.27 (t, J=10.4 Hz, 0.3H), 2.01-1.84 (m, 1H), 1.10 (d, J=5.7 Hz, 3H), 0.95-0.86 (m, 2H), 0.78-0.57 (m, 2H). ESI MS [M+H]⁻ for C₂₈H₂₉N₇O, calcd 480.2, found 480.2.

### Example 33: 4-cyclopropyl-2-(1-methylpiperidin-4-yl)-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

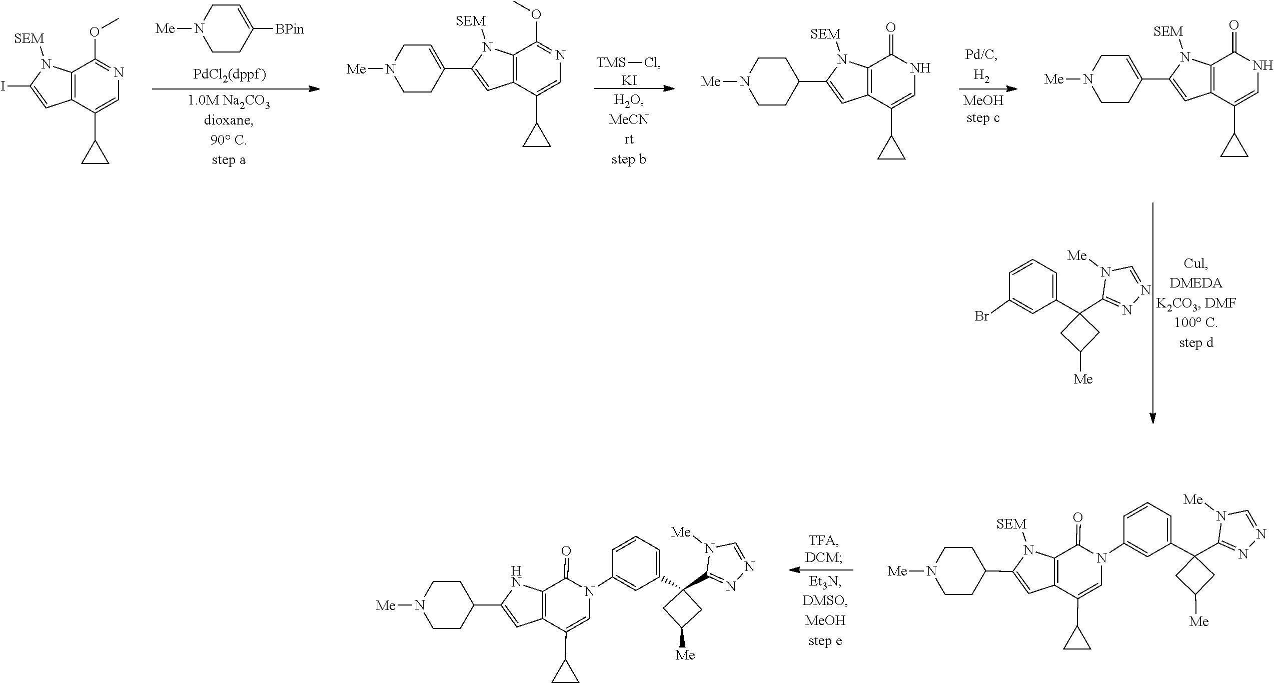

Step a: To a flask charged with 2-[(4-cyclopropyl-2-iodo-7-methoxypyrrolo[2,3-c]pyridin-1-yl)methoxy]ethyl-trimethylsilane (321 mg, 0.72 mmol), 1-Methyl-1,2,3,6-tetrahydropyridine-4-boronic acid pinacol ester (191 mg, 0.86 mmol), Pd(dppf)Cl₂ (53 mg, 0.072 mmol) was added dioxane (7.2 mL) and Na₂CO₃ (1.0M/H₂O, 2.9 mL, 2.9 mmol). The mixture was heated to 90° C. overnight. After cooling to room temperature, the reaction was diluted with EtOAc and washed with water and brine. The organics were dried with MgSO₄ and concentrated under reduced pressure. Column chromatography (SiO₂, 0 to 10% MeOH/DCM) afforded the desired product.

Step b: To a flask charged with the product from step a (282 mg, 0.68 mmol) and KI (181 mg, 1.09 mmol) was added MeCN (6.8 mL) and water (0.012 mL) followed by TMS-Cl (0.14 mL, 1.09 mmol). After stirring overnight, the reaction was diluted with EtOAc and washed with 1.0M Na₂CO₃ and brine. The organics were dried with MgSO₄ and concentrated under reduced pressure. HPLC purification (C18, gradient MeCN/H₂O with 0.1% HCO₂H) afforded the desired product.

Step c: To a solution of the product from step b (67 mg, 0.17 mmol) in MeOH (3.4 mL) under nitrogen in a was added Pd/C (10 wt %-Pd, 35 mg). The vessel was pressured to 50 psi with hydrogen in a shaker vessel and mixed for 4 hours. Subsequently, the mixture was filtered through Celite® and washed with MeOH and EtOAc. The filtrate was concentrated and used without further purification.

Steps d-e: The title compound was completed in a similar manner to Example 31, steps f-g. ¹H NMR (400 MHz, DMSO-d₆) δ 12.04 (s, 1H), 8.46 (s, 1H), 7.57-7.45 (m, 1H), 7.41-7.33 (m, 3H), 7.31-7.19 (m, 2H), 6.81 (s, 1H), 6.26 (s, 1H), 3.53 (d, J=12.4 Hz, 2H), 3.12 (dt, J=35.5, 11.4 Hz, 4H), 3.01-2.78 (m, 6H), 2.34-2.20 (m, 2H), 1.98-1.73 (m, 3H), 1.07 (d, J=5.0 Hz, 4H), 0.88-0.74 (m, 3H), 0.65 (q, J=6.1, 5.6 Hz, 3H). ESI MS [M+H]⁺ for C₃₀H36N₆₀, calcd 497.3, found 497.3.

### Example 34 and 35: 4-Cyclopropyl-3-(1-methyl-1H-imidazol-5-yl)-6-{3-[(1R,3S)-3-methyl-1-(4-methyl-4H-1,2,4-triazol-3-yl)cyclobutyl]phenyl}-1H,6H,7H-pyrrolo[2,3-c]pyridin-7-one and 4-Cyclopropyl-3-(1-methyl-1H-imidazol-5-yl)-6-{3-[(1S,3R)-3-methyl-1-(4-methyl-4H-1,2,4-triazol-3-yl)cyclobutyl]phenyl}-1H,6H,7H-pyrrolo[2,3-c]pyridin-7-one

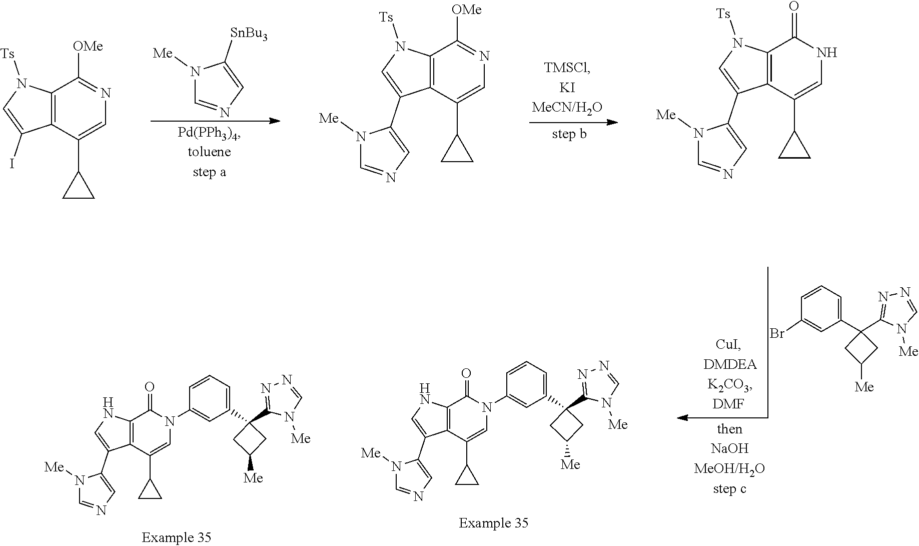

Step a: To a solution of 4-cyclopropyl-3-iodo-7-methoxy-1-(4-methylphenyl)sulfonylpyrrolo[2,3-c]pyridine (234 mg, 0.50 mmol, 1.0 equiv.) in toluene (2.5 mL) was added 1-methyl-5-tributylstannyl-1H-imidazole (204 mg, 0.55 mmol, 1.1 equiv.) and Pd(PPh₃)₄ (57.8 mg, 0.050 mmol, 10 mol %). The resulting mixture was heated at 110° C. for overnight. After cooling to room temperature, the reaction mixture was concentrated. The crude material was directly used in the next step.

Step b: To a mixture of the crude product from step a in MeCN/H₂O (4:1 v/v, 2.5 mL) was added TMSCl (109 mg, 1.0 mmol) and KI (166 mg, 1.0 mmol). The resulting mixture was heated at 80° C. for 2 h when LCMS showed a completion of the demethylation. After cooling to room temperature, the reaction mixture was concentrated to dryness. The crude residue was purified by column chromatography (SiO₂, MeOH in DCM, 0 to 10%) to give the desired product.

Step c: To a solution of the product from step b (40.8 mg, 0.10 mmol, 1.0 equiv.), 3-[1-(3-bromophenyl)-3-methylcyclobutyl]-4-methyl-1,2,4-triazole (diastereomeric mixture in 3:1 ratio, 45.9 mg, 0.15 mmol, 1.5 equiv.) in DMF (1.0 mL) was added CuI (19.0 mg, 0.10 mmol, 1.0 equiv.), N,N′-dimethylethylenediamine (17.6 mg, 0.20 mmol, 2.0 equiv.) and K₂CO₃ (41.5 mg, 0.30 mmol, 3.0 equiv.). The resulting mixture was heated at 110° C. for 2 h when LCMS showed a full conversion to the product with tosyl protecting group. After cooling to the room temperature, the reaction mixture was diluted with EtOAc and washed with water twice. The organic phase was then washed with brine, dried over Na₂SO₄ and concentrated. The crude residue was then dissolved in THF (2 mL) followed by the addition of 2 mL of 1M NaOH aqueous solution. The resulting mixture was stirring at room temperature overnight with a clean detosylation showed by LCMS, and then extracted with EtOAc twice. The combined organic phase was washed with brine, dried over Na₂SO₄ and concentrated. The residue was then purified by column chromatography (SiO₂, MeOH in DCM, 0 to 10%) and further purified by HPLC to afford the title compounds.

Example 34: First eluting diastereomer (10:1 d.r.) ¹H NMR (400 MHz, CDCl₃) δ 10.77 (s, 1H), 7.96 (s, 1H), 7.61 (s, 1H), 7.55-7.41 (m, 2H), 7.39 (s, 1H), 7.32-7.23 (m, 2H), 7.10 (s, 1H), 6.68 (s, 1H), 3.50 (s, 3H), 3.27 (s, 3H), 2.93-2.81 (m, 2H), 2.76-2.62 (m, 3H), 1.48 (p, J=7.7 Hz, 1H), 1.14 (d, J=4.8 Hz, 3H), 0.53-0.44 (m, 2H), 0.44-0.35 (m, 2H).

Example 35: First eluting diastereomer (2.3:1 d.r.) ¹H NMR (400 MHz, CDCl₃) δ 10.55 (s, 1H), 8.03 (s, 0.72H), 7.97 (s, 0.28H), 7.61 (s, 1H), 7.53-7.19 (m, 5H), 7.10 (s, 1H), 6.68 (s, 1H), 3.51 (s, 3H), 3.32 (s, 2.14H), 3.27 (s, 0.86H), 3.23-2.27 (m, 5H), 1.48 (p, J=7.4 Hz, 1H), 1.13 (d, J=6.5 Hz, 3H), 0.54-0.44 (m, 2H), 0.46-0.33 (m, 2H).

### Example 36 and 37: 6-{3-[(1R,3S)-3-Methyl-1-(4-methyl-4H-1,2,4-triazol-3-yl)cyclobutyl]phenyl}-4-(trifluoromethyl)-1H,6H,7H-pyrrolo[2,3-c]pyridin-7-one and 6-{3-[(1S,3R)-3-Methyl-1-(4-methyl-4H-1,2,4-triazol-3-yl)cyclobutyl]phenyl}-4-(trifluoromethyl)-1H,6H,7H-pyrrolo[2,3-c]pyridin-7-one

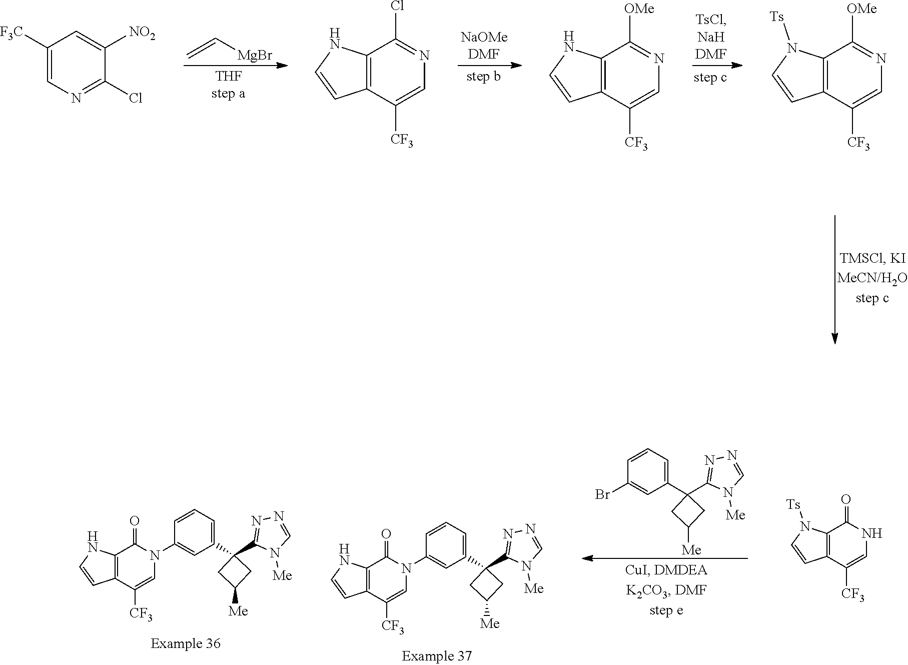

Step a: To a solution of 2-chloro-3-nitro-5-(trifluoromethyl)pyridine (5.3 g, 23 mmol, 1.0 equiv.) in THF (50 mL) was added vinylmagnesium bromide (1M in THF, 76 mL, 76 mmol, 3.3 equiv.) at −78° C. over 30 min. The resulting mixture was stirred at this temperature for another 30 min before quenched with saturated NH₄Cl aqueous solution. The mixture was then extracted with EtOAc twice. The combined organic phase was washed with brine, dried over Na₂SO₄ and concentrated. The residue was then purified by column chromatography (SiO₂, EtOAc in hexanes, 0 to 25%) to give the desired product.

Step b: To a solution of the product from step a (297 mg, 1.3 mmol, 1.0 equiv.) in DMF (6.5 mL) was added NaOMe (792 mg, 13 mmol, 10 equiv.). The resulting mixture was heated at 130° C. for 1 h. After cooling back to room temperature, the mixture was diluted with EtOAc and washed sequentially with water twice and bine once. The organic phase was then dried over Na₂SO₄ and concentrated. The residue was then purified by column chromatography (SiO₂, EtOAc in hexanes, 0 to 20%) to give the desired product.

Step c: To a solution of the product from step b (225 mg, 1.0 mmol, 1.0 equiv.) in DMF (3.0 mL) was added NaH (60 wt % in mineral oil, 80.0 mg, 2.0 mmol, 2.0 equiv.) at 0° C. The resulting mixture was stirred at this temperature for 10 min before the addition of 4-toluenesulfonyl chloride (286 mg, 1.5 mmol, 1.5 equiv.). The reaction mixture was then raised to room temperature and stirred for overnight. The reaction was then quenched with water and diluted with EtOAc. The organic phase was separated and washed with water twice. The organic solution was then washed with brine, dried over Na₂SO₄ and concentrated. The crude residue was purified by column chromatography (SiO₂, EtOAc in hexanes, 0 to 15%) to give the desired product.

Step d: To a mixture of the product from step c (320 mg, 0.86 mmol, 1.0 equiv.) in MeCN/H₂O (4:1 v/v, 5.0 mL) was added TMSCl (141 mg, 0.16 mL, 1.3 mmol, 1.5 equiv.) and KI (216 mg, 1.3 mmol, 1.5 equiv.). The resulting mixture was heated at 80° C. for 2 h when LCMS showed a completion of the demethylation. After cooling to room temperature, the reaction mixture was concentrated to dryness. The crude residue was purified by column chromatography (SiO₂, MeOH in DCM, 0 to 10%) to give the desired product.

Step e: To a solution of the product from step d (107 mg, 0.30 mmol, 1.0 equiv.), 3-[1-(3-bromophenyl)-3-methylcyclobutyl]-4-methyl-1,2,4-triazole (diastereomeric mixture in 3:1 ratio, 138 mg, 0.45 mmol, 1.5 equiv.) in DMF (3.0 mL) was added CuI (57.1 mg, 0.30 mmol, 1.0 equiv.), N,N′-dimethylethylenediamine (52.9 mg, 0.60 mmol, 2.0 equiv.) and K₂CO₃ (124 mg, 0.90 mmol, 3.0 equiv.). The resulting mixture was heated at 110° C. for 2 h when LCMS showed a full conversion to the desired product with a clean detosylation. After cooling to the room temperature, the reaction mixture was diluted with EtOAc and washed with water twice. The organic phase was then washed with brine, dried over Na₂SO₄ and concentrated. The crude residue was purified by column chromatography (SiO₂, MeOH in DCM, 0 to 10%) and further purified by HPLC to afford the title compounds.

Example 35: First eluting diastereomer (10:1 d.r.) ¹H NMR (400 MHz, CDCl₃) δ 10.13 (s, 1H), 7.99 (s, 1H), 7.59-7.41 (m, 4H), 7.39-7.32 (m, 1H), 7.32-7.25 (m, 1H), 6.60 (s, 1H), 3.27 (s, 2.73H), 3.21 (s, 0.27H), 2.95-2.84 (m, 2H), 2.78-2.63 (m, 3H), 1.15 (d, J=5.1 Hz, 3H).

Example 36: First eluting diastereomer (2.5:1 d.r.) ¹H NMR (400 MHz, CDCl₃) δ 10.43 (s, 1H), 8.06 (s, 0.73H), 8.00 (s, 0.27H), 7.58-7.28 (m, 6H), 6.59 (s, 1H), 3.32 (s, 2.1H), 3.27 (s, 0.9H), 3.24-2.26 (m, 5H), 1.14 (d, J=6.4 Hz, 3H).

### Example 38: 7-oxo-6-{3-[(1r,3s)-3-methyl-1-(4-methyl-4H-1,2,4-triazol-3-yl)cyclobutyl]phenyl}-1H,6H,7H-pyrrolo[2,3-c]pyridine-4-carbonitrile

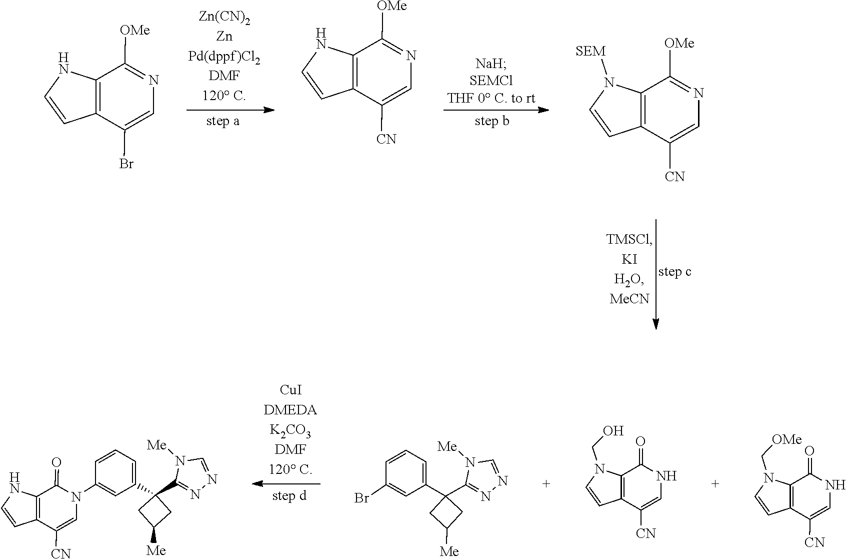

Step a: The desired product was prepared in a similar manner to Example 27 step b.

Step b: The desired product was prepared in a similar manner to Example 7, step a.

Step c: The desired product was prepared in a similar manner to Example 29, step c.

Step d: The desired product was prepared in a similar manner to Example 33, step c to afford the title compound as a 2.3:1 mixture of diastereomers. ¹H NMR (400 MHz, CDCl₃) δ 10.91 (s, 0.3H), 10.75 (d, J=19.3 Hz, 0.7H), 8.01 (d, J=2.4 Hz, 1H), 7.62-7.50 (m, 1H), 7.54-7.43 (m, 2H), 7.47-7.20 (m, 3H), 6.69-6.58 (m, 1H), 3.36-3.14 (m, 3H), 2.93-2.81 (m, 2H), 2.69 (dt, J=10.3, 8.0 Hz, 2.8H), 2.32 (td, J=9.3, 2.7 Hz, 0.2H), 1.18-1.09 (m, 3H). ESI MS [M+H]⁻ for C₂₂H₂₁N₆O, calcd 385.2, found 385.2.

### Example 39: 4-cyclopropyl-6-{3-[3-methyl-1-(4-methyl-4H-1,2,4-triazol-3-yl)cyclobutyl]phenyl}-2-{[(1-methylcyclobutyl)amino]methyl}-1H,6H,7H-pyrrolo[2,3-c]pyridin-7-one

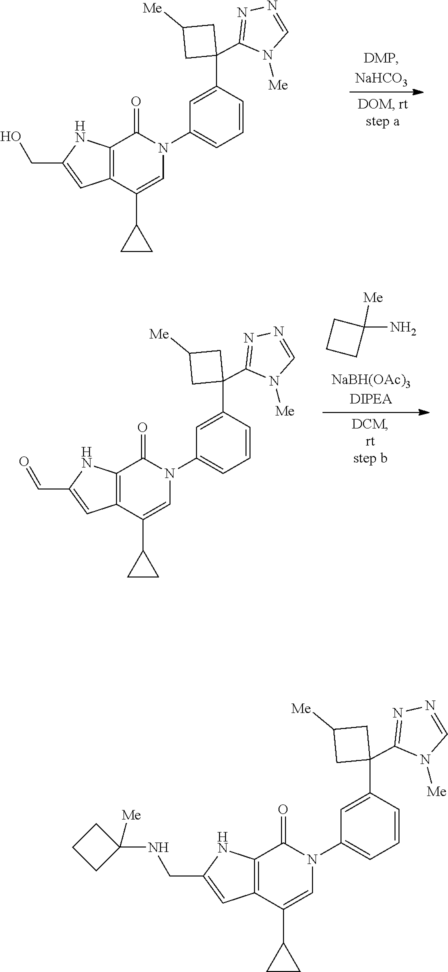

Step a: To 4-cyclopropyl-2-(hydroxymethyl)-6-{3-[3-methyl-1-(4-methyl-4H-1,2,4-triazol-3-yl)cyclobutyl]phenyl}-1H,6H,7H-pyrrolo[2,3-c]pyridin-7-one (172 mg, 0.4009 mmol, 1.0 equiv.) from Example 8 in DCM (4 mL, 0.1 M) was added NaHCO₃ (168 mg, 2.0045 mmol, 5.0 equiv.) and DMP (510 mg, 1.2027 mmol, 3.0 equiv.). The resulting mixture was stirred at rt for 3 h. The reaction was quenched with sat. aq. NH₄Cl solution, the organic phase was separated, and the aqueous layer was extracted with DCM. The combined organic phase was dried over Na₂SO₄, concentrated and the crude residue was purified by column chromatography (SiO₂, MeOH in DCM, 0 to 5%) to afford 4-cyclopropyl-6-[3-[3-methyl-1-(4-methyl-1,2,4-triazol-3-yl)cyclobutyl]phenyl]-7-oxo-1H-pyrrolo[2,3-c]pyridine-2-carbaldehyde.

Step b: To the product of step a (40 mg, 0.09367 mmol, 1.0 equiv.) in DCM (3 mL, 0.03 M) was added 1-methylcyclobutan-1-amine hydrochloride (23 mg, 0.1873 mmol, 2.0 equiv.) and DIPEA (0.05 mL, 0.2342 mmol, 2.5 equiv.) and the mixture was stirred at rt for 10 mins. NaBH(OAc)₃ (52 mg, 0.2342 mmol, 2.5 equiv.) was added and the mixture was stirred at rt for 12 h. The reaction was quenched with NaHCO₃ sat., the organic phase was separated, and the aqueous layer was extracted with EtOAc. The combined organic phase was dried over Na₂SO₄, concentrated and the crude residue was purified by column chromatography (SiO₂, MeOH in DCM, 0 to 25%) to give a crude mixture. The resulting mixture was further was purified by prep-HPLC to furnish the title compound as a white solid. ¹H NMR (400 MHz, CDCl₃) δ 7.97 (s, 1H), 7.52 (dd, J=8.4, 7.3 Hz, 1H), 7.43-7.34 (m, 2H), 7.30-7.26 (m, 1H), 6.65 (d, J=1.2 Hz, 1H), 6.43 (s, 1H), 3.98 (s, 2H), 3.26 (s, 3H), 2.96-2.80 (m, 2H), 2.72-2.59 (m, 3H), 2.17 (d, J=10.3 Hz, 2H), 1.91-1.72 (m, 5H), 1.40 (s, 3H), 1.14 (d, J=5.7 Hz, 3H), 0.86-0.78 (m, 2H), 0.62-0.52 (m, 2H). ESI MS [M+H]⁺ for C₃₀H₃₆N₆O, calcd 497.6, found 497.1.

### Example 40: 4-cyclopropyl-2-({[(3R,4R)-4-hydroxyoxolan-3-yl]amino}methyl)-6-{3-[3-methyl-1-(4-methyl-4H-1,2,4-triazol-3-yl)cyclobutyl]phenyl}-1H,6H,7H-pyrrolo[2,3-c]pyridin-7-one

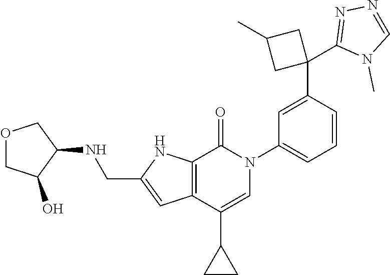 [SMILES: CC1CC(c2cccc(-n3cc(C4CC4)c4cc(CN[C@@H]5COC[C@@H]5O)[nH]c4c3=O)c2)(c2nncn2C)C1]

The title compound was prepared in a similar fashion to that described for Example 39 from (3R,4R)-4-aminotetrahydrofuran-3-ol. ¹H NMR (400 MHz, CDCl₃) δ 12.02 (s, 1H), 7.99 (s, 1H), 7.59-7.47 (m, 2H), 7.32 (dd, J=6.5, 1.9 Hz, 2H), 6.69 (d, J=1.2 Hz, 1H), 6.39 (s, 1H), 3.98-3.86 (m, 2H), 3.83-3.65 (m, 4H), 3.29 (s, 3H), 3.13 (t, J=3.6 Hz, 2H), 2.97-2.82 (m, 2H), 2.77-2.62 (m, 3H), 2.2-1.80 (m, 3H), 1.15 (d, J=5.2 Hz, 3H), 0.86 (dt, J=9.4, 3.1 Hz, 2H), 0.67-0.58 (m, 2H). ESI MS [M+H]⁻ for C₂₉H₃₅N₆O₃, calcd 515.6, found 515.1.

### Example 41: 4-cyclopropyl-6-{3-[(1r,3s)-3-methyl-1-(4-methyl-4H-1,2,4-triazol-3-yl)cyclobutyl]phenyl}-2-({[1-(trifluoromethyl)cyclopropyl]amino}methyl)-1H,6H,7H-pyrrolo[2,3-c]pyridin-7-one

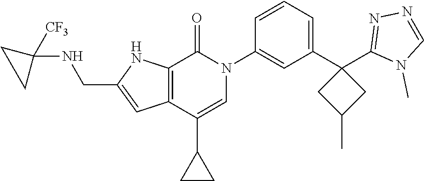 [SMILES: CC1CC(c2cccc(-n3cc(C4CC4)c4cc(CNC5(C(F)(F)F)CC5)[nH]c4c3=O)c2)(c2nncn2C)C1]

The title compound was prepared in a similar fashion to that described for Example 39 from 1-(trifluoromethyl)cyclopropanamine hydrochloride and 3-[1-(3-bromophenyl)-3-methylcyclobutyl]-4-methyl-1,2,4-triazole from General Procedure 1. ¹H NMR (400 MHz, CDCl₃) δ 9.62 (s, 1H), 7.96 (s, 1H), 7.49-7.43 (m, 2H), 7.38-7.33 (m, 1H), 7.24 (dd, J=1.9, 1.2 Hz, 1H), 6.75 (d, J=1.2 Hz, 1H), 6.39 (d, J=2.2 Hz, 1H), 4.18 (s, 2H), 3.27 (s, 3H), 2.87 (t, J=5.3 Hz, 2H), 2.75-2.61 (m, 3H), 1.94-1.81 (m, 1H), 1.14 (d, J=5.8 Hz, 3H), 1.09-0.99 (m, 2H), 0.92 (d, J=4.8 Hz, 2H), 0.90-0.76 (m, 2H), 0.70-0.56 (m, 2H). ESI MS [M+H]⁺ for C₂₉H₃₁F3N₆O, calcd 537.6, found 537.1.

### Example 42: 4-cyclopropyl-2-({[1-(hydroxymethyl)cyclobutyl]amino}methyl)-6-{3-[3-methyl-1-(4-methyl-4H-1,2,4-triazol-3-yl)cyclobutyl]phenyl}-1H,6H,7H-pyrrolo[2,3-c]pyridin-7-one

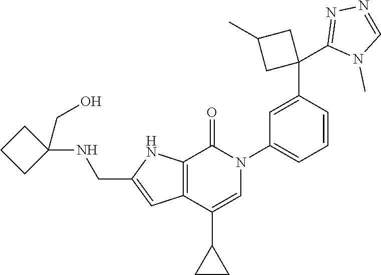 [SMILES: CC1CC(c2cccc(-n3cc(C4CC4)c4cc(CNC5(CO)CCC5)[nH]c4c3=O)c2)(c2nncn2C)C1]

The title compound was prepared in a similar fashion to that described for Example 39 from (1-aminocyclobutyl)methanol hydrochloride. ¹H NMR (400 MHz, CDCl₃) δ 8.04 (m, 1H), 7.51 (m, 7.7 Hz, 2H), 7.31 (s, 1H), 7.20 (m, 1H), 6.62 (d, J=1.2 Hz, 1H), 6.35 (d, J=16.4 Hz, 1H), 4.07 (d, J=13.2 Hz, 2H), 3.76 (d, J=6.4 Hz, 2H), 3.31 (d, J=16.7 Hz, 3H), 3.19 (td, J=8.2, 2.7 Hz, 1H), 2.91-2.84 (m, 2H), 2.69 (d, J=6.6 Hz, 2H), 2.33 (dd, J=11.9, 9.3 Hz, 1H), 2.23 (s, 3H), 2.05-1.94 (m, 2H), 1.87 (t, J=12.2 Hz, 1H), 1.82-1.71 (m, 2H), 1.14 (t, J=6.0 Hz, 3H), 0.87-0.73 (m, 2H), 0.54 (d, J=5.5 Hz, 2H). ESI MS [M+H]⁻ for C₃₀H₃₇N₆O₂, calcd 513.6, found 513.1.

### Example 43: 4-cyclopropyl-6-{3-[3-methyl-1-(4-methyl-4H-1,2,4-triazol-3-yl)cyclobutyl]phenyl}-2-{[(2S′)-2-methylpiperidin-1-yl]methyl}-1H,6H,7H-pyrrolo[2,3-c]pyridin-7-one

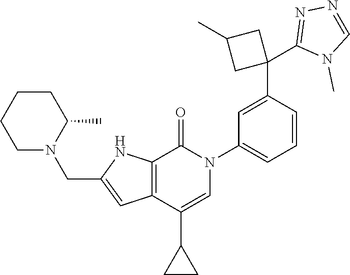 [SMILES: CC1CC(c2cccc(-n3cc(C4CC4)c4cc(CN5CCCC[C@@H]5C)[nH]c4c3=O)c2)(c2nncn2C)C1]

The title compound was prepared in a similar fashion to that described for Example 39 from (2S)-2-methylpiperidine hydrochloride. ¹H NMR (400 MHz, CDCl₃) δ 10.86 (s, 1H), 8.00 (d, J=25.5 Hz, 1H), 7.44 (d, J=7.5 Hz, 2H), 7.35 (d, J=7.9 Hz, 1H), 7.20 (d, J=8.1 Hz, 1H), 6.74 (s, 1H), 6.43 (s, 1H), 4.30 (d, J=14.3 Hz, 1H), 3.61 (d, J=14.3 Hz, 1H), 3.30 (d, J=25.3 Hz, 3H), 2.89 (d, J=11.2 Hz, 3H), 2.67 (d, J=5.3 Hz, 3H), 2.32-2.11 (m, 3H), 1.87 (h, J=6.3, 5.2 Hz, 1H), 1.77 (d, J=14.2 Hz, 1H), 1.68-1.61 (m, 2H), 1.55 (t, J=10.3 Hz, 1H), 1.40 (d, J=8.3 Hz, 1H), 1.27 (d, J=6.4 Hz, 3H), 1.14 (d, J=4.7 Hz, 3H), 0.86 (d, J=8.1 Hz, 2H), 0.63 (d, J=5.2 Hz, 2H). ESI MS [M+H]⁺ for C₃₁H₃₉N₆O, calcd 511.6, found 511.1.

### Example 44: 2-({2-azabicyclo[4.1.0]heptan-2-yl}methyl)-4-cyclopropyl-6-{3-[3-methyl-1-(4-methyl-4H-1,2,4-triazol-3-yl)cyclobutyl]phenyl}-1H,6H,7H-pyrrolo[2,3-c]pyridin-7-one

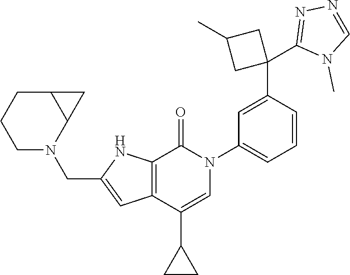 [SMILES: CC1CC(c2cccc(-n3cc(C4CC4)c4cc(CN5CCCC6CC65)[nH]c4c3=O)c2)(c2nncn2C)C1]

The title compound was prepared in a similar fashion to that described for Example 39 from 2-azabicyclo[4.1.0]heptane hydrochloride. H NMR (400 MHz, CDCl₃) δ 9.56 (s, 1H), 7.96 (s, 1H), 7.52-7.39 (m, 3H), 7.34 (d, J=7.9 Hz, 1H), 6.74 (s, 1H), 6.41 (s, 1H), 4.05-3.64 (m, 2H), 3.27 (s, 3H), 2.88 (s, 2H), 2.67 (d, J=7.7 Hz, 3H), 2.51 (d, J=8.9 Hz, 1H), 2.40-2.22 (m, 1H), 2.18 (d, J=18.6 Hz, 2H), 2.04-1.95 (m, 1H), 1.87 (d, J=8.4 Hz, 2H), 1.44 (d, J=16.1 Hz, 1H), 1.14 (d, J=5.0 Hz, 3H), 1.05 (t, J=7.9 Hz, 1H), 0.87 (d, J=8.2 Hz, 2H), 0.65 (d, J=5.4 Hz, 2H), 0.44-0.28 (m, 2H). ESI MS [M+H]⁻ for C₃₁H₃₇N₆O, calcd 509.6, found 509.1.

### Example 45: 4-cyclopropyl-6-{3-[3-methyl-1-(4-methyl-4H-1,2,4-triazol-3-yl)cyclobutyl]phenyl}-2-{[(2R)-2-methylmorpholin-4-yl]methyl}-1H,6H,7H-pyrrolo[2,3-c]pyridin-7-one

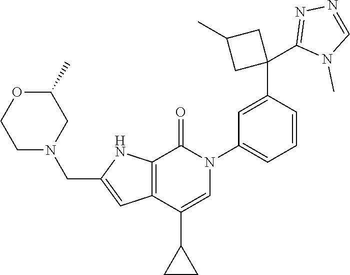 [SMILES: CC1CC(c2cccc(-n3cc(C4CC4)c4cc(CN5CCO[C@H](C)C5)[nH]c4c3=O)c2)(c2nncn2C)C1]

The title compound was prepared in a similar fashion to that described for Example 39 from (R)-2-methylmorpholine. ¹H NMR (400 MHz, CDCl₃) δ 9.71-9.51 (m, 1H), 8.00 (d, J=25.5 Hz, 1H), 7.54-7.33 (m, 3H), 7.21 (d, J=7.6 Hz, 1H), 6.74 (s, 1H), 6.41 (s, 1H), 3.85 (dd, J=10.9, 2.6 Hz, 1H), 3.76-3.57 (m, 4H), 3.30 (d, J=24.7 Hz, 3H), 3.23-3.13 (m, 1H), 2.95-2.83 (m, 2H), 2.70 (m, 4H), 2.38-2.16 (m, 1H), 1.99-1.83 (m, 2H), 1.13 (d, J=6.2 Hz, 6H), 0.87 (d, J=8.0 Hz, 2H), 0.64 (d, J=5.3 Hz, 2H). ESI MS [M+H]⁺ for C₃₀H₃₆N₆O₂, calcd 513.6, found 513.1.

### Example 46: 4-cyclopropyl-6-{3-[3-methyl-1-(4-methyl-4H-1,2,4-triazol-3-yl)cyclobutyl]phenyl}-2-({[2-(trifluoromethoxy)ethyl]amino}methyl)-1H,6H,7H-pyrrolo[2,3-c]pyridin-7-one

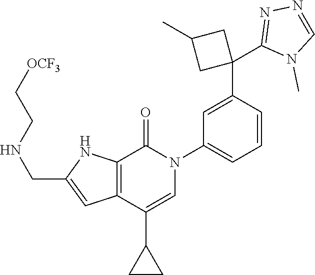 [SMILES: CC1CC(c2cccc(-n3cc(C4CC4)c4cc(CNCCOC(F)(F)F)[nH]c4c3=O)c2)(c2nncn2C)C1]

The title compound was prepared in a similar fashion to that described for Example 39 from 2-(trifluoromethoxy)ethanamine hydrochloride. ¹H NMR (400 MHz, CDCl₃) δ 10.03 (s, 1H), 7.96 (s, 1H), 7.49-7.41 (m, 2H), 7.38 (ddd, J=7.9, 1.9, 1.1 Hz, 1H), 7.27 (dd, J=2.1, 1.0 Hz, 1H), 6.74 (d, J=1.2 Hz, 1H), 6.41 (s, 1H), 4.15-4.01 (m, 2H), 3.99 (d, J=0.8 Hz, 2H), 3.30 (d, J=24.2 Hz, 3H), 3.21-3.10 (m, 1H), 2.89 (dt, J=10.8, 5.7 Hz, 4H), 2.68 (d, J=7.7 Hz, 3H), 1.92-1.83 (m, 1H), 1.24-1.05 (m, 3H), 0.98-0.80 (m, 2H), 0.70-0.55 (m, 2H). ESI MS [M+H]⁺ for C₂₈H₃₂N₆O₂, calcd 541.6, found 541.1.

### Example 47: 2-{[cyclopentyl(methyl)amino]methyl}-4-cyclopropyl-6-{3-[3-methyl-1-(4-methyl-4H-1,2,4-triazol-3-yl)cyclobutyl]phenyl}-1H,6H,7H-pyrrolo[2,3-c]pyridin-7-one

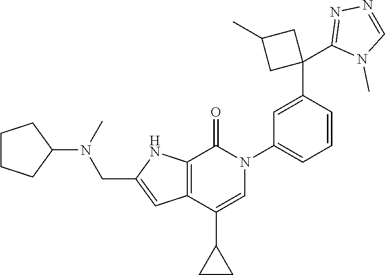 [SMILES: CC1CC(c2cccc(-n3cc(C4CC4)c4cc(CN(C)C5CCCC5)[nH]c4c3=O)c2)(c2nncn2C)C1]

The title compound was prepared in a similar fashion to that described for Example 39 from N-methylcyclopentanamine. ¹H NMR (400 MHz, CDCl₃) δ 9.64 (s, 1H), 7.99 (d, J=25.2 Hz, 1H), 7.53-7.39 (m, 2H), 7.34 (d, J=8.7 Hz, 1H), 7.22 (s, 1H), 6.74 (s, 1H), 6.37 (s, 1H), 3.70 (s, 2H), 3.31 (d, J=26.0 Hz, 3H), 2.88 (s, 2H), 2.80 (t, J=8.0 Hz, 1H), 2.67 (d, J=7.7 Hz, 2H), 2.20 (s, 3H), 1.87 (m, 4H), 1.71 (s, 2H), 1.48 (m, 4H), 1.14 (d, J=4.8 Hz, 3H), 0.87 (d, J=8.3 Hz, 2H), 0.65 (d, J=5.3 Hz, 2H). ESI MS [M+H]⁺ for C₃₁H₃₉N₆O, calcd 511.6, found 511.1.

### Example 48: 4-cyclopropyl-6-{3-[3-methyl-1-(4-methyl-4H-1,2,4-triazol-3-yl)cyclobutyl]phenyl}-2-{[(3S′)-3-(trifluoromethyl)piperidin-1-yl]methyl}-1H,6H,7H-pyrrolo[2,3-c]pyridin-7-one

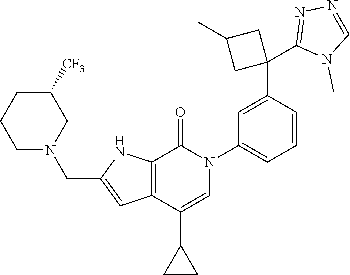 [SMILES: CC1CC(c2cccc(-n3cc(C4CC4)c4cc(CN5CCC[C@H](C(F)(F)F)C5)[nH]c4c3=O)c2)(c2nncn2C)C1]

The title compound was prepared in a similar fashion to that described for Example 39 from (R)-2-methylmorpholine. ¹H NMR (400 MHz, CDCl₃) δ 9.68 (s, 1H), 8.00 (d, J=25.2 Hz, 1H), 7.45 (d, J=8.3 Hz, 2H), 7.36 (d, J=7.9 Hz, 1H), 7.22 (d, J=8.0 Hz, 1H), 6.75 (s, 1H), 6.40 (s, 1H), 3.68 (s, 2H), 3.30 (d, J=24.7 Hz, 3H), 3.05 (d, J=10.7 Hz, 1H), 2.87 (d, J=10.9 Hz, 3H), 2.68 (t, J=7.6 Hz, 3H), 2.33 (d, J=21.2 Hz, 1H), 2.12-1.94 (m, 3H), 1.91-1.84 (m, 1H), 1.74 (s, 1H), 1.59 (d, J=12.7 Hz, 1H), 1.37-1.21 (m, 1H), 1.14 (d, J=4.4 Hz, 3H), 0.87 (d, J=8.0 Hz, 2H), 0.65 (d, J=5.3 Hz, 2H). ESI MS [M+H]⁺ for C₃₂H₃₆F23N₆O, calcd 565.6, found 565.1.

### Example 49: 4-cyclopropyl-6-{3-[3-methyl-1-(4-methyl-4H-1,2,4-triazol-3-yl)cyclobutyl]phenyl}-2-{[(oxan-3-yl)amino]methyl}-1H,6H,7H-pyrrolo[2,3-c]pyridin-7-one

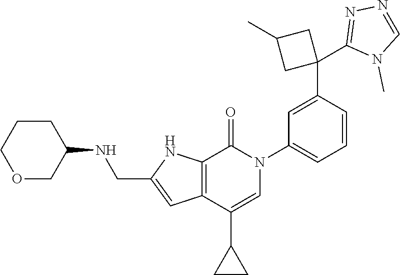

The title compound was prepared in a similar fashion to that described for Example 39 from (R)-tetrahydro-2H-pyran-3-amine hydrochloride. ¹H NMR (400 MHz, CDCl₃) δ 8.00 (d, J=24.7 Hz, 1H), 7.56-7.45 (m, 1H), 7.43 (d, J=2.0 Hz, 1H), 7.39 (d, J=8.0 Hz, 1H), 7.26-7.18 (m, 1H), 6.72 (d, J=1.2 Hz, 1H), 6.42 (s, 1H), 4.13-4.00 (m, 2H), 3.85 (dd, J=11.2, 3.5 Hz, 1H), 3.74 (dd, J=10.1, 5.7 Hz, 1H), 3.51-3.40 (m, 1H), 3.31 (d, J=23.5 Hz, 4H), 2.89 (d, J=4.0 Hz, 2H), 2.76-2.61 (m, 4H), 1.89-1.80 (m, 3H), 1.73 (dd, J=12.3, 6.5 Hz, 1H), 1.63-1.42 (m, 2H), 1.13 (dd, J=6.2, 4.4 Hz, 3H), 0.90-0.80 (m, 2H), 0.61 (q, J=5.1 Hz, 2H). ESI MS [M+H]⁺ for C₃₀H₃₇N₆O₂, calcd 513.6, found 513.1.

### Example 50: 2-({6-azaspiro[2.5]octan-6-yl}methyl)-4-cyclopropyl-6-{3-[3-methyl-1-(4-methyl-4H-1,2,4-triazol-3-yl)cyclobutyl]phenyl}-1H,6H,7H-pyrrolo[2,3-c]pyridin-7-one

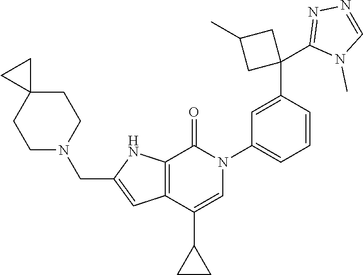 [SMILES: CC1CC(c2cccc(-n3cc(C4CC4)c4cc(CN5CCC6(CC5)CC6)[nH]c4c3=O)c2)(c2nncn2C)C1]

The title compound was prepared in a similar fashion to that described for Example 39 from 6-azaspiro[2.5]octane. ¹H NMR (400 MHz, CDCl₃) δ 10.07 (s, 1H), 7.99 (d, J=25.4 Hz, 1H), 7.51-7.39 (m, 2H), 7.35 (d, J=7.9 Hz, 1H), 7.21 (d, J=8.3 Hz, 1H), 6.74 (s, 1H), 6.41 (s, 1H), 3.75 (s, 2H), 3.30 (d, J=25.5 Hz, 3H), 2.92-2.82 (m, 2H), 2.67 (d, J=7.5 Hz, 2H), 2.58 (m, 3H), 1.91 (m, 4H), 1.45 (m, 4H), 1.14 (d, J=4.7 Hz, 3H), 0.87 (d, J=8.1 Hz, 2H), 0.64 (d, J=5.3 Hz, 2H), 0.30 (s, 4H). ESI MS [M+H]⁺ for C₃₂H₃₉N₆O, calcd 523.6, found 523.1.

### Example 51: 4-cyclopropyl-6-{3-[3-methyl-1-(4-methyl-4H-1,2,4-triazol-3-yl)cyclobutyl]phenyl}-2-{[(1R,4R)-2-oxa-5-azabicyclo[2.2.1]heptan-5-yl]methyl}-1H,6H,7H-pyrrolo[2,3-c]pyridin-7-one

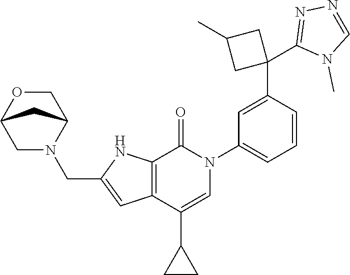 [SMILES: CC1CC(c2cccc(-n3cc(C4CC4)c4cc(CN5C[C@H]6C[C@@H]5CO6)[nH]c4c3=O)c2)(c2nncn2C)C1]

The title compound was prepared in a similar fashion to that described for Example 39 from (R)-tetrahydro-2H-pyran-3-amine hydrochloride. H NMR (400 MHz, CDCl₃) δ 9.64 (s, 1H), 7.99 (d, J=25.4 Hz, 1H), 7.45 (d, J=7.8 Hz, 2H), 7.36 (d, J=7.9 Hz, 1H), 7.21 (d, J=8.0 Hz, 1H), 6.74 (s, 1H), 6.38 (s, 1H), 4.44 (s, 1H), 4.08 (d, J=7.9 Hz, 1H), 3.92 (q, J=14.3 Hz, 2H), 3.67 (d, J=7.9 Hz, 1H), 3.50 (s, 1H), 3.30 (d, J=25.4 Hz, 3H), 2.89 (dd, J=12.7, 6.9 Hz, 3H), 2.66 (dt, J=20.2, 9.7 Hz, 3H), 1.89 (q, J=7.9, 6.0 Hz, 2H), 1.78 (d, J=9.9 Hz, 1H), 1.13 (t, J=5.0 Hz, 3H), 0.87 (d, J=8.2 Hz, 2H), 0.64 (d, J=5.3 Hz, 2H). ESI MS [M+H]⁺ for C₃₀H35N₆₀₂, calcd 511.6, found 511.1.

### Example 52: 2-({2-azabicyclo[2.2.1]heptan-2-yl}methyl)-4-cyclopropyl-6-{3-[3-methyl-1-(4-methyl-4H-1,2,4-triazol-3-yl)cyclobutyl]phenyl}-1H,6H,7H-pyrrolo[2,3-c]pyridin-7-one

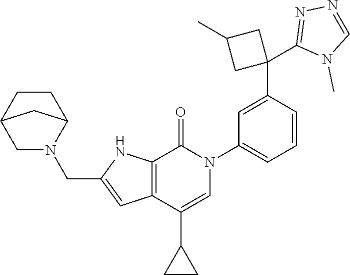 [SMILES: CC1CC(c2cccc(-n3cc(C4CC4)c4cc(CN5CC6CCC5C6)[nH]c4c3=O)c2)(c2nncn2C)C1]

The title compound was prepared in a similar fashion to that described for Example 39 from 2-azabicyclo[2.2.1]heptane. H NMR (400 MHz, CDCl₃) δ 12.32 (s, 1H), 8.01 (d, J=25.6 Hz, 1H), 7.43 (dd, J=14.3, 6.6 Hz, 2H), 7.34 (d, J=8.0 Hz, 1H), 7.20 (d, J=8.0 Hz, 1H), 6.74 (s, 1H), 6.52 (s, 1H), 4.22 (q, J=13.6 Hz, 2H), 3.77 (s, 1H), 3.29 (d, J=24.9 Hz, 3H), 3.03 (s, 2H), 2.87 (s, 2H), 2.67 (d, J=8.7 Hz, 4H), 2.16 (d, J=11.0 Hz, 1H), 2.05-1.95 (m, 2H), 1.85 (d, J=5.8 Hz, 1H), 1.72 (dd, J=25.1, 13.7 Hz, 2H), 1.62 (d, J=11.5 Hz, 2H), 1.14 (d, J=4.8 Hz, 3H), 0.86 (d, J=8.2 Hz, 2H), 0.63 (d, J=5.3 Hz, 2H). ESI MS [M+H]⁺ for C₃₁H₃₇N₆O, calcd 509.6, found 509.1.

### Example 53: 4-cyclopropyl-6-{3-[3-methyl-1-(4-methyl-4H-1,2,4-triazol-3-yl)cyclobutyl]phenyl}-2-({6-oxa-3-azabicyclo[3.1.1]heptan-3-yl}methyl)-1H,6H,7H-pyrrolo[2,3-c]pyridin-7-one

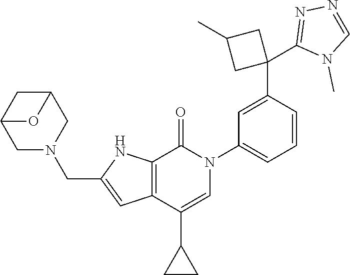 [SMILES: CC1CC(c2cccc(-n3cc(C4CC4)c4cc(CN5CC6CC(C5)O6)[nH]c4c3=O)c2)(c2nncn2C)C1]

The title compound was prepared in a similar fashion to that described for Example 39 from 6-oxa-3-azabicyclo[3.1.1]heptane. ¹H NMR (400 MHz, CDCl₃) δ 9.70 (s, 1H), 7.96 (s, 1H), 7.45 (d, J=8.1 Hz, 2H), 7.37 (d, J=8.0 Hz, 1H), 7.26 (s, 1H), 6.74 (s, 1H), 6.45 (s, 1H), 4.51 (d, J=6.2 Hz, 2H), 3.92 (s, 2H), 3.30 (d, J=25.0 Hz, 3H), 3.10 (d, J=11.4 Hz, 2H), 3.03 (q, J=6.8 Hz, 1H), 2.87 (d, J=11.1 Hz, 3H), 2.68 (d, J=7.5 Hz, 2H), 2.42 (d, J=8.0 Hz, 1H), 1.94-1.82 (m, 2H), 1.14 (d, J=4.6 Hz, 3H), 0.88 (d, J=8.0 Hz, 2H), 0.65 (d, J=5.3 Hz, 2H). ESI MS [M+H]⁺ for C₃₀H₃₄N₆O₂, calcd 511.6, found 511.1.

### Example 54: 4-cyclopropyl-2-{I[cyclopropyl(methyl)amino]methyl}-6-{3-[3-methyl-1-(4-methyl-4H-1,2,4-triazol-3-yl)cyclobutyl]phenyl}-1H,6H,7H-pyrrolo[2,3-c]pyridin-7-one

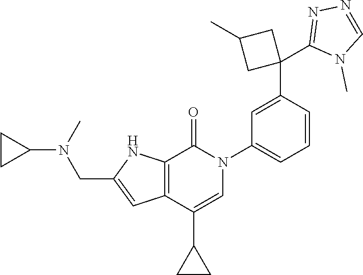 [SMILES: CC1CC(c2cccc(-n3cc(C4CC4)c4cc(CN(C)C5CC5)[nH]c4c3=O)c2)(c2nncn2C)C1]

The title compound was prepared in a similar fashion to that described for Example 39 from N-methylcyclopropanamine hydrochloride. ¹H NMR (400 MHz, CDCl₃) δ 8.00 (d, J=25.6 Hz, 1H), 7.51-7.41 (m, 2H), 7.40-7.32 (m, 1H), 7.25-7.18 (m, 1H), 6.74 (d, J=1.2 Hz, 1H), 6.51-6.40 (m, 1H), 3.91 (s, 2H), 3.30 (d, J=24.1 Hz, 3H), 2.92-2.81 (m, 2H), 2.72-2.62 (m, 2H), 2.37 (s, 3H), 1.89 (m, 2H), 1.27-1.08 (m, 3H), 0.93-0.80 (m, 2H), 0.72-0.61 (m, 2H), 0.59-0.51 (m, 4H). ESI MS [M+H]⁺ for C₂₉H₃₅N₆O, calcd 483.6, found 483.1.

### Example 55: 4-cyclopropyl-2-[(dimethylamino)methyl]-6-{3-[3-methyl-1-(4-methyl-4H-1,2,4-triazol-3-yl)cyclobutyl]phenyl}-1H,6H,7H-pyrrolo[2,3-c]pyridin-7-one

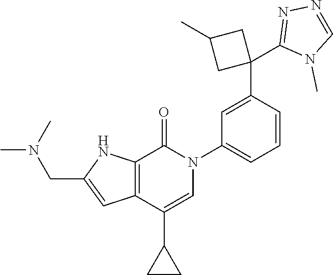 [SMILES: CC1CC(c2cccc(-n3cc(C4CC4)c4cc(CN(C)C)[nH]c4c3=O)c2)(c2nncn2C)C1]

The title compound was prepared in a similar fashion to that described for Example 39 from dimethylamine hydrochloride. ¹H NMR (400 MHz, CDCl₃) δ 11.00 (s, 1H), 8.02 (d, J=25.9 Hz, 1H), 7.48 (q, J=6.8, 5.9 Hz, 1H), 7.44-7.37 (m, 2H), 7.30-7.21 (m, 1H), 6.73 (s, 1H), 6.54 (s, 1H), 3.88 (s, 2H), 3.31 (d, J=21.5 Hz, 3H), 2.96-2.85 (m, 2H), 2.67 (d, J=7.8 Hz, 3H), 2.44 (s, 7H), 1.87 (dd, J=9.4, 4.4 Hz, 1H), 1.13 (t, J=5.0 Hz, 3H), 0.87 (d, J=8.0 Hz, 2H), 0.63 (d, J=5.3 Hz, 2H). ESI MS [M+H]⁺ for C₂₇H₃₂N₆O, calcd 457.6, found 457.1.

### Example 56: 4-cyclopropyl-2-({[(3S)-oxolan-3-yl]amino}methyl)-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

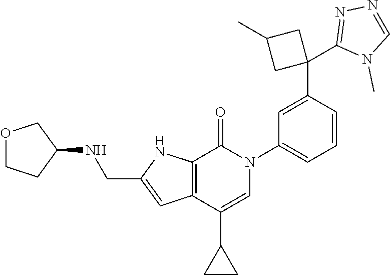 [SMILES: CC1CC(c2cccc(-n3cc(C4CC4)c4cc(CN[C@H]5CCOC5)[nH]c4c3=O)c2)(c2nncn2C)C1]

The title compound was prepared in a similar fashion to that described for Example 39 from (S)-tetrahydrofuran-3-amine hydrochloride. ¹H NMR (400 MHz, Methanol-d4) δ 8.40 (d, J=30.2 Hz, 1H), 7.71-7.48 (m, 2H), 7.44-7.18 (m, 2H), 6.89 (dd, J=4.8, 1.2 Hz, 1H), 6.85 (s, 1H), 4.45 (s, 2H), 4.14-3.96 (m, 3H), 3.86 (dd, J=10.7, 5.4 Hz, 1H), 3.78 (q, J=8.3 Hz, 1H), 3.39 (d, J=8.7 Hz, 3H), 3.04-2.95 (m, 2H), 2.64 (dq, J=19.4, 9.3, 8.8 Hz, 2H), 2.55-2.37 (m, 2H), 2.17-2.04 (m, 1H), 2.03-1.91 (m, 1H), 1.39 (dd, J=6.7, 3.3 Hz, 1H), 1.17 (d, J=6.2 Hz, 3H), 1.04-0.87 (m, 2H), 0.74-0.64 (m, 2H). ESI MS [M+H]⁺ for C₂₉H₃₄N₆O₂, calcd 499.6, found 499.1.

### Example 57: 4-cyclopropyl-2-{[(3R)-4,4-difluoro-3-methylpiperidin-1-yl]methyl}-6-{3-[3-methyl-1-(4-methyl-4H-1,2,4-triazol-3-yl)cyclobutyl]phenyl}-1H,6H,7H-pyrrolo[2,3-c]pyridin-7-one

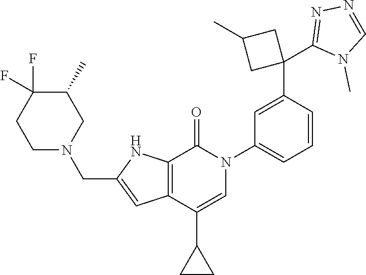 [SMILES: CC1CC(c2cccc(-n3cc(C4CC4)c4cc(CN5CCC(F)(F)[C@H](C)C5)[nH]c4c3=O)c2)(c2nncn2C)C1]

The title compound was prepared in a similar fashion to that described for Example 39 from (R)-4,4-difluoro-3-methylpiperidine hydrochloride. ¹H NMR (400 MHz, CDCl₃) δ 9.65 (s, 1H), 8.00 (d, J=25.5 Hz, 1H), 7.49-7.40 (m, 2H), 7.38 (d, J=7.9 Hz, 1H), 7.24 (d, J=10.2 Hz, 1H), 6.74 (d, J=3.6 Hz, 1H), 6.41 (s, 1H), 3.68 (s, 2H), 3.30 (d, J=24.4 Hz, 3H), 2.93-2.78 (m, 2H), 2.71 (dd, J=22.8, 8.1 Hz, 3H), 2.35 (dt, J=22.5, 10.6 Hz, 2H), 2.15-2.01 (m, 3H), 1.91-1.64 (m, 2H), 1.13 (t, J=5.2 Hz, 3H), 1.00 (d, J=5.6 Hz, 3H), 0.88 (d, J=8.1 Hz, 2H), 0.64 (d, J=5.3 Hz, 2H). ESI MS [M+H]⁺ for C₃₁H₃₇F2N₆O, calcd 547.6, found 547.1.

### Example 58: 4-cyclopropyl-2-[(3,3-difluoroazetidin-1-yl)methyl]-6-{3-[3-methyl-1-(4-methyl-4H-1,2,4-triazol-3-yl)cyclobutyl]phenyl}-1H,6H,7H-pyrrolo[2,3-c]pyridin-7-one

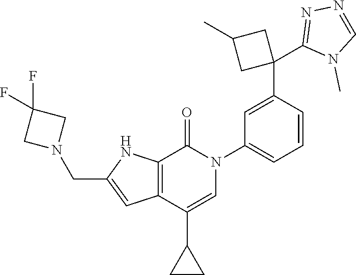 [SMILES: CC1CC(c2cccc(-n3cc(C4CC4)c4cc(CN5CC(F)(F)C5)[nH]c4c3=O)c2)(c2nncn2C)C1]

The title compound was prepared in a similar fashion to that described for Example 39 from 3,3-difluoroazetidine hydrochloride. ¹H NMR (400 MHz, CDCl₃) δ 9.91 (s, 1H), 7.99 (d, J=25.5 Hz, 1H), 7.45 (dd, J=16.4, 8.7 Hz, 2H), 7.38 (d, J=8.0 Hz, 1H), 7.22 (d, J=7.8 Hz, 1H), 6.75 (s, 1H), 6.42 (s, 1H), 3.86 (s, 2H), 3.64 (t, J=12.0 Hz, 3H), 3.30 (d, J=24.5 Hz, 3H), 2.88 (s, 2H), 2.68 (d, J=7.7 Hz, 3H), 1.86 (dt, J=8.4, 5.3 Hz, 1H), 1.14 (d, J=4.7 Hz, 3H), 0.87 (d, J=8.1 Hz, 2H), 0.64 (d, J=5.3 Hz, 2H). ESI MS [M+H]⁺ for C₂₈H₃₁F₂N₆O, calcd 505.6, found 505.1.

### Example 59: 4-cyclopropyl-2-{[(1S,4S)-2,5-diazabicyclo[2.2.1]heptan-2-yl]methyl}-6-{3-[3-methyl-1-(4-methyl-4H-1,2,4-triazol-3-yl)cyclobutyl]phenyl}-1H,6H,7H-pyrrolo[2,3-c]pyridin-7-one

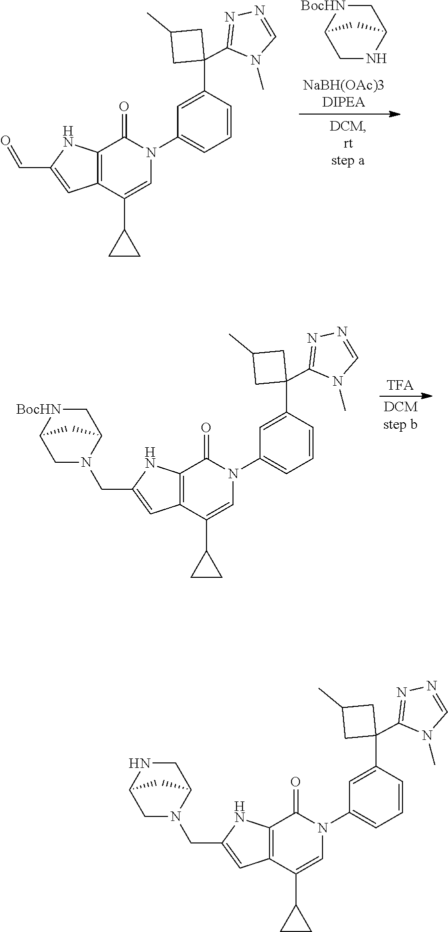

Step a: The product of step a was prepared in a similar fashion to that described for Example 39 from (1S,4S)-2-boc-2,5-diazabicyclo[2.2.1]heptane.

Step b: The product of step a (28 mg, 0.04684 mmol, 1.0 equiv.) was suspended in DCM (3 mL, 0.015 M) followed by TFA (1.0 mL, excess) at rt and the mixture was stirred for 1 h. 2 mL of 1 M aq. NaOH and 20 mL of H₂O were added, and the mixture was stirred at rt for 0.5 h. The organic phase was separated, and the aqueous layer was extracted with EtOAc. The combined organic phase was dried over Na₂SO₄, concentrated and the crude residue was purified by column chromatography (SiO₂, MeOH in DCM, 0 to 20%) to give a crude mixture. The resulting mixture was further was purified by prep-HPLC to furnish the title compound. H NMR (400 MHz, CDCl₃) δ 10.52 (s, 1H), 7.98 (s, 1H), 7.45 (q, J=9.0, 8.1 Hz, 2H), 7.39 (d, J=7.0 Hz, 2H), 6.73 (s, 1H), 6.37 (s, 1H), 4.06 (s, 1H), 3.98 (d, J=14.1 Hz, 1H), 3.85 (d, J=14.1 Hz, 1H), 3.58-3.47 (m, 2H), 3.26 (s, 3H), 3.11 (d, J=10.9 Hz, 1H), 3.01 (d, J=10.9 Hz, 1H), 2.88 (d, J=10.0 Hz, 4H), 2.66 (d, J=7.3 Hz, 3H), 2.01 (d, J=10.7 Hz, 1H), 1.88 (dd, J=22.6, 8.8 Hz, 2H), 1.19-1.04 (m, 3H), 0.86 (d, J=8.2 Hz, 2H), 0.63 (d, J=5.3 Hz, 2H). ESI MS [M+H]⁺ for C₃₀H₃₅N₇O, calcd 510.7, found 510.1.

### Example 60: 4-cyclopropyl-6-{3-[3-methyl-1-(4-methyl-4H-1,2,4-triazol-3-yl)cyclobutyl]phenyl}-2-{[(1R,5S)-3-oxa-6-azabicyclo[3.1.1]heptan-6-yl]methyl}-1H,6H,7H-pyrrolo[2,3-c]pyridin-7-one

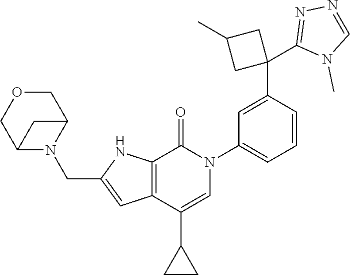 [SMILES: CC1CC(c2cccc(-n3cc(C4CC4)c4cc(CN5C6COCC5C6)[nH]c4c3=O)c2)(c2nncn2C)C1]

The title compound was prepared in a similar fashion to that described for Example 39 from 3-oxa-6-azabicyclo[3.1.1]heptane hydrochloride. ¹H NMR (400 MHz, CDCl₃) δ 9.62 (s, 1H), 7.99 (d, J=25.6 Hz, 1H), 7.44 (d, J=8.6 Hz, 2H), 7.35 (d, J=8.0 Hz, 1H), 7.21 (d, J=7.6 Hz, 1H), 6.73 (s, 1H), 6.40 (s, 1H), 4.27 (d, J=10.9 Hz, 2H), 4.02 (s, 2H), 3.77 (d, J=10.9 Hz, 2H), 3.52 (d, J=6.0 Hz, 2H), 3.30 (d, J=24.8 Hz, 3H), 2.86 (d, J=8.6 Hz, 2H), 2.67 (d, J=7.7 Hz, 4H), 1.87 (d, J=8.4 Hz, 2H), 1.14 (d, J=4.8 Hz, 3H), 0.87 (d, J=8.1 Hz, 2H), 0.64 (d, J=5.5 Hz, 2H). ESI MS [M+H]⁺ for C₃₀H34N₆O2, calcd 511.7, found 511.1.

### Example 61: 4-cyclopropyl-2-{[(3R)-3,4-dimethylpiperazin-1-yl]methyl}-6-{3-[3-methyl-1-(4-methyl-4H-1,2,4-triazol-3-yl)cyclobutyl]phenyl}-1H,6H,7H-pyrrolo[2,3-c]pyridin-7-one

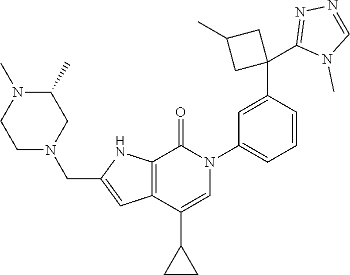 [SMILES: CC1CC(c2cccc(-n3cc(C4CC4)c4cc(CN5CCN(C)[C@H](C)C5)[nH]c4c3=O)c2)(c2nncn2C)C1]

The title compound was prepared in a similar fashion to that described for Example 39 from (R)-1,2-dimethylpiperazine. ¹H NMR (400 MHz, CDCl₃) δ 9.61 (s, 1H), 8.00 (d, J=25.5 Hz, 1H), 7.45 (d, J=8.4 Hz, 2H), 7.37 (d, J=8.0 Hz, 1H), 7.22 (s, 1H), 6.74 (s, 1H), 6.41 (s, 1H), 3.67 (s, 2H), 3.30 (d, J=24.6 Hz, 3H), 2.96 (s, 1H), 2.88 (d, J=3.6 Hz, 1H), 2.78 (m, 2H), 2.68 (m, 3H), 2.48 (s, 2H), 2.41 (s, 3H), 2.31 (t, J=10.3 Hz, 1H), 2.11 (d, J=26.0 Hz, 1H), 1.87 (d, J=6.4 Hz, 1H), 1.14 (d, J=4.5 Hz, 6H), 0.95-0.80 (m, 2H), 0.64 (d, J=5.3 Hz, 2H). ESI MS [M+H]⁺ for C₃₁H₃₉N₇O, calcd 526.7, found 526.1.

### Example 62: 4-cyclopropyl-2-{[(3R)-3,4-dimethylpiperazin-1-yl]methyl}-6-{3-[3-methyl-1-(4-methyl-4H-1,2,4-triazol-3-yl)cyclobutyl]phenyl}-1H,6H,7H-pyrrolo[2,3-c]pyridin-7-one

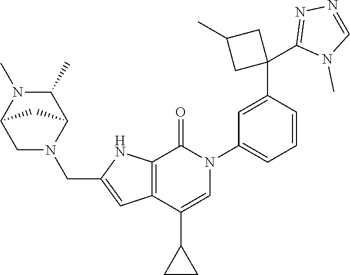 [SMILES: CC1CC(c2cccc(-n3cc(C4CC4)c4cc(CN5C[C@@H]6C[C@H]5[C@@H](C)N6C)[nH]c4c3=O)c2)(c2nncn2C)C1]

The title compound was prepared in a similar fashion to that described for Example 39 from (1S,4S)-2-methyl-2,5-diazabicyclo[2.2.1]heptane. ¹H NMR (400 MHz, CDCl₃δ 9.70 (s, 1H), 7.99 (d, J=25.4 Hz, 1H), 7.44 (d, J=8.9 Hz, 2H), 7.35 (d, J=7.9 Hz, 1H), 7.21 (d, J=8.2 Hz, 1H), 6.74 (s, 1H), 6.36 (s, 1H), 3.96-3.77 (m, 2H), 3.32 (d, J=9.4 Hz, 3H), 3.27 (s, 3H), 2.89 (dd, J=28.5, 11.4 Hz, 4H), 2.69 (dd, J=17.0, 8.4 Hz, 5H), 2.44 (s, 3H), 1.86 (d, J=6.8 Hz, 1H), 1.14 (d, J=4.7 Hz, 3H), 0.86 (d, J=8.2 Hz, 2H), 0.64 (d, J=5.4 Hz, 2H). ESI MS [M+H]⁻ for C₃₁H₃₈N₇O, calcd 524.7, found 524.1.

### Example 63: 2-({5-azaspiro[2.4]heptan-5-yl}methyl)-4-cyclopropyl-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

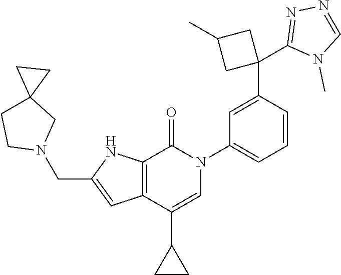 [SMILES: CC1CC(c2cccc(-n3cc(C4CC4)c4cc(CN5CCC6(CC6)C5)[nH]c4c3=O)c2)(c2nncn2C)C1]

The title compound was prepared in a similar fashion to that described for Example 39 from (1S,4S)-2-methyl-2,5-diazabicyclo[2.2.1]heptane. H NMR (400 MHz, CDCl₃) δ 8.01 (s, 1H), 7.56-7.43 (m, 2H), 7.39 (t, J=2.0 Hz, 1H), 7.28 (ddd, J=7.7, 2.0, 1.2 Hz, 1H), 6.71 (d, J=1.2 Hz, 1H), 6.65 (s, 1H), 4.17 (s, 2H), 3.31 (s, 3H), 3.13 (s, 2H), 2.88 (d, J=7.0 Hz, 4H), 2.72-2.60 (m, 2H), 1.94-1.82 (m, 3H), 1.22-0.98 (m, 3H), 0.92-0.76 (m, 2H), 0.62 (dt, J=3.5, 1.7 Hz, 7H). ESI MS [M+H]⁺ for C₃₁H₃₇N₆O, calcd 509.7, found 509.1.

### Example 64: 4-cyclopropyl-2-{[(3S)-3-fluoropiperidin-1-yl]methyl}-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

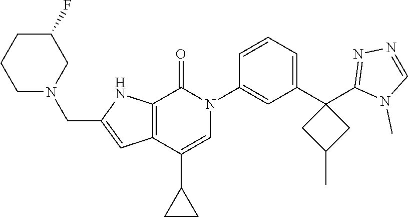 [SMILES: CC1CC(c2cccc(-n3cc(C4CC4)c4cc(CN5CCC[C@H](F)C5)[nH]c4c3=O)c2)(c2nncn2C)C1]

The title compound was prepared in a similar fashion to that described for Example 39 from (3S)-3-fluoropiperidine. ¹H NMR (400 MHz, CDCl₃) δ 7.99 (s, 1H), 7.54-7.43 (m, 1H), 7.44-7.34 (m, 2H), 7.27 (s, 1H), 6.75 (d, J=1.2 Hz, 1H), 6.54-6.41 (m, 1H), 4.71 (d, J=47.6 Hz, 1H), 3.87 (s, 3H), 3.31 (d, J=23.2 Hz, 4H), 2.93-2.82 (m, 1H), 2.78-2.56 (m, 4H), 2.50 (s, 2H), 1.88 (dtd, J=12.8, 9.0, 7.3, 4.4 Hz, 3H), 1.66 (d, J=21.9 Hz, 2H), 1.13 (dd, J=6.2, 4.3 Hz, 3H), 0.94-0.81 (m, 2H), 0.76-0.58 (m, 2H). ESI MS [M+H]⁺ for C₃₀H₃₆FN₆O, calcd 515.6, found 515.1.

### Example 65: 4-cyclopropyl-2-{[(3S,5S)-3,5-difluoropiperidin-1-yl]methyl}-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

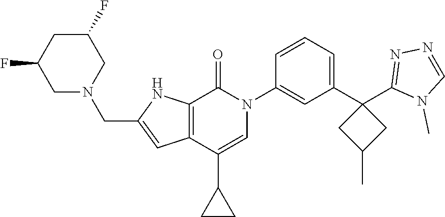 [SMILES: CC1CC(c2cccc(-n3cc(C4CC4)c4cc(CN5C[C@@H](F)C[C@H](F)C5)[nH]c4c3=O)c2)(c2nncn2C)C1]

The title compound was prepared in a similar fashion to that described for Example 39 from (3S,5S)-3,5-difluoropiperidine hydrochloride. ¹H NMR (400 MHz, CDCl₃) δ 9.92 (s, 1H), 7.98 (s, 1H), 7.50-7.43 (m, 2H), 7.37 (dt, J=8.4, 1.3 Hz, 1H), 7.28 (dd, J=2.0, 1.0 Hz, 1H), 6.75 (d, J=1.2 Hz, 1H), 6.42 (d, J=2.1 Hz, 1H), 4.95 (dd, J=6.3, 3.4 Hz, 1H), 4.83 (dd, J=6.2, 3.4 Hz, 1H), 3.91-3.68 (m, 2H), 3.28 (s, 3H), 2.94-2.84 (m, 2H), 2.80-2.55 (m, 8H), 2.07 (ddt, J=23.1, 14.0, 4.2 Hz, 2H), 1.95-1.83 (m, 1H), 1.14 (d, J=5.4 Hz, 3H), 0.92-0.81 (m, 2H), 0.72-0.56 (m, 2H). ESI MS [M+H]⁺ for C₃₀H₃₅F₂N₆O, calcd 533.6, found 533.1.

### Example 66: 4-cyclopropyl-2-[(3,3-difluoropiperidin-1-yl)methyl]-6-{3-[(1r)-3-methyl-1-(4-methyl-4H-1,2,4-triazol-3-yl)cyclobutyl]phenyl}-1H,6H,7H-pyrrolo[2,3-c]pyridin-7-one

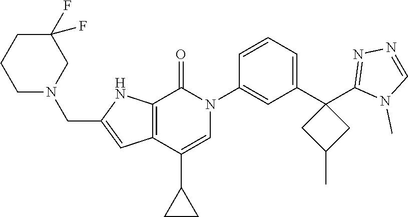 [SMILES: CC1CC(c2cccc(-n3cc(C4CC4)c4cc(CN5CCCC(F)(F)C5)[nH]c4c3=O)c2)(c2nncn2C)C1]

The title compound was prepared in a similar fashion to that described for Example 39 from 3,3-difluoropiperidine hydrochloride. ¹H NMR (400 MHz, CDCl₃) δ 9.53 (s, 1H), 7.96 (s, 1H), 7.55-7.41 (m, 2H), 7.36 (dt, J=8.1, 1.3 Hz, 1H), 7.27 (d, J=1.2 Hz, 1H), 6.75 (d, J=1.2 Hz, 1H), 6.41 (d, J=2.2 Hz, 1H), 3.73 (s, 2H), 3.27 (s, 3H), 2.95-2.82 (m, 1H), 2.70 (dd, J=15.3, 8.4 Hz, 4H), 2.48 (d, J=5.6 Hz, 2H), 1.97-1.83 (m, 3H), 1.78 (d, J=6.2 Hz, 3H), 1.14 (d, J=5.7 Hz, 3H), 0.98-0.82 (m, 2H), 0.69-0.61 (m, 2H). ESI MS [M+H]⁺ for C₃₀H₃₅F₅N₆₀, calcd 533.65, found 533.1. ESI MS [M+H]⁺ for C₃₀H₃₅F₂N₆O, calcd 533.6, found 533.1.

### Example 67: 4-cyclopropyl-2-{[(3R,5S)-3,5-difluoropiperidin-1-yl]methyl}-6-{3-[(1r)-3-methyl-1-(4-methyl-4H-1,2,4-triazol-3-yl)cyclobutyl]phenyl}-1H,6H,7H-pyrrolo[2,3-c]pyridin-7-one

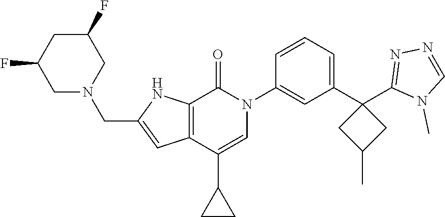 [SMILES: CC1CC(c2cccc(-n3cc(C4CC4)c4cc(CN5C[C@H](F)C[C@H](F)C5)[nH]c4c3=O)c2)(c2nncn2C)C1]

The title compound was prepared in a similar fashion to that described for Example 39 from (3R,5S)-3,5-difluoropiperidine hydrochloride. ¹H NMR (400 MHz, CDCl₃) δ 9.86 (s, 1H), 8.01 (s, 1H), 7.57-7.34 (m, 3H), 7.27 (s, 1H), 6.76 (d, J=1.2 Hz, 1H), 6.45 (d, J=2.1 Hz, 1H), 4.78-4.69 (m, 1H), 4.64-4.55 (m, 1H), 3.85 (s, 2H), 3.42-3.15 (m, 3H), 3.02-2.84 (m, 3H), 2.76-2.62 (m, 3H), 2.54 (dt, J=11.3, 7.2 Hz, 2H), 2.36 (td, J=18.4, 13.5 Hz, 1H), 1.92-1.81 (m, 1H), 1.64 (p, J=7.7 Hz, 3H), 1.14 (d, J=5.5 Hz, 3H), 0.91-0.83 (m, 2H), 0.67-0.61 (m, 2H). ESI MS [M+H]⁺ for C₃₀H₃₅F₂N₆O, calcd 533.6, found 533.1.

### Example 68: 4-cyclopropyl-2-{[(2S)-4-methyl-2-(propan-2-yl)piperazin-1-yl]methyl}-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

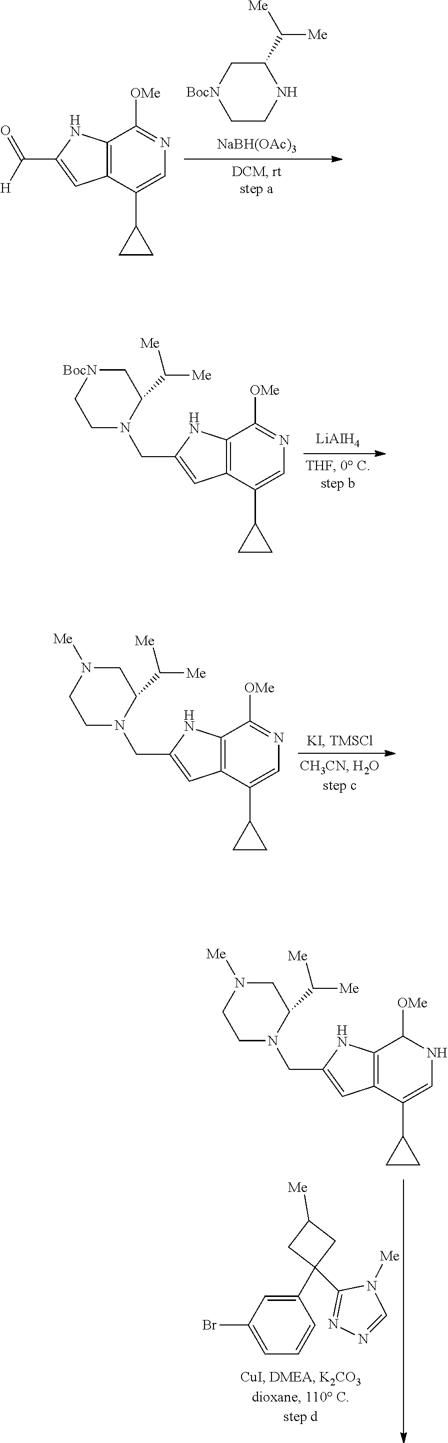

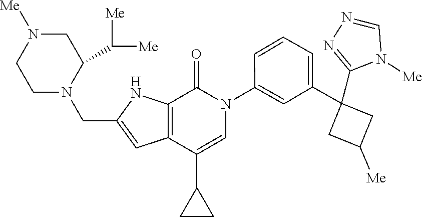 [SMILES: CC1CC(c2cccc(-n3cc(C4CC4)c4cc(CN5CCN(C)C[C@@H]5C(C)C)[nH]c4c3=O)c2)(c2nncn2C)C1]

Step a: To the solution of 4-cyclopropyl-7-methoxy-1H-pyrrolo[2,3-c]pyridine-2-carbaldehyde (220 mg, 1.0 mmol, 1.0 equiv.) in DCM (5 mL, 0.2 M) was added (S)-1-Boc-3-isopropylpiperazine (342 mg, 1.5 mmol, 1.5 equiv.) and DIPEA (0.35 mL, 2.0 mmol, 2.0 equiv.) and the mixture was stirred at rt for 10 mins. NaBH(OAc)₃ (444 mg, 2.0 mmol, 2.0 equiv.) was added and the mixture was stirred at rt for 12 h. The reaction was quenched with NaHCO₃ sat., the organic phase was separated, and the aqueous layer was extracted with EtOAc. The combined organic phase was dried over Na₂SO₄, concentrated and the crude residue was purified by column chromatography (SiO₂, MeOH in DCM, 0 to 15%) to give tert-butyl (3S)-4-[(4-cyclopropyl-7-methoxy-1H-pyrrolo[2,3-c]pyridin-2-yl)methyl]-3-propan-2-ylpiperazine-1-carboxylate.

Step b: To solution of the product of step a (126 mg, 0.2940 mmol, 1.0 equiv.) in THF (5 mL, 0.06 M) was added lithium aluminum hydride solution (1.2 ml, 1.1760 mmol, 4.0 equiv., 1.0 M) slowly at 0° C. The resulting mixture was stirred at 0° C. for 2 h. The reaction was quenched with sat. aq. NH₄Cl solution, the organic phase was separated, and the aqueous layer was extracted with EtOAc. The combined organic phase was dried over Na₂SO₄ and concentrated to afford a crude residue. The crude product 4-cyclopropyl-7-methoxy-2-[[(2S)-4-methyl-2-propan-2-ylpiperazin-1-yl]methyl]-1H-pyrrolo[2,3-c]pyridine was used for the next step without purification.

Step c: To a solution of the product from step b (44 mg, 0.1283 mmol, 1.0 equiv.) and KI (34 mg, 0.2052 mmol, 1.6 equiv.) in CH₃CN (3 mL, 0.04 M) was added TMSCl (22 mg, 0.2052 mmol, 1.6 equiv.) dropwise at rt. H₂O (0.01 ml) was added into the solution. The resulting mixture was stirred at rt for 12 h. The mixture was then quenched with H₂O. The organic phase was separated, and the aqueous phase was extracted with EtOAc, the combined organic phase was then washed with brine, dried over Na₂SO₄ and concentrated and the crude residue was purified by column chromatography (SiO₂, MeOH in DCM, 0 to 20%) to give 4-cyclopropyl-2-[[(2S)-4-methyl-2-propan-2-ylpiperazin-1-yl]methyl]-1,6-dihydropyrrolo[2,3-c]pyridin-7-one.

Step d: The title compound was prepared in a similar fashion to that described for Example 70 from 3-((1s,3s)1-(3-bromophenyl)-3-methylcyclobutyl)-4-methyl-1,2,4-triazole from General Procedure 2. ¹H NMR (400 MHz, CDCl₃) δ 9.48 (s, 1H), 7.96 (s, 1H), 7.51-7.42 (m, 2H), 7.35 (dt, J=8.2, 1.2 Hz, 1H), 7.26-7.23 (m, 1H), 6.75 (d, J=1.2 Hz, 1H), 6.40 (d, J=2.1 Hz, 1H), 4.22 (d, J=14.4 Hz, 1H), 3.31 (d, J=14.4 Hz, 1H), 3.27 (s, 3H), 2.92-2.75 (m, 4H), 2.68 (d, J=7.1 Hz, 2H), 2.35 (s, 5H), 2.28-2.00 (m, 3H), 1.91-1.82 (m, 2H), 1.14 (d, J=5.5 Hz, 3H), 0.99 (d, J=6.9 Hz, 3H), 0.94 (d, J=6.9 Hz, 3H), 0.89-0.82 (m, 2H), 0.70-0.61 (m, 2H). ESI MS [M+H]⁺ for C₃₃H₄₄N₇O, calcd 554.8, found 554.1.

### Example 69: N-[(4-cyclopropyl-7-oxo-6-{3-[(1r,3s)-3-methyl-1-(4-methyl-4H-1,2,4-triazol-3-yl)cyclobutyl]phenyl}-1H,6H,7H-pyrrolo[2,3-c]pyridin-2-yl)methyl]-N-methylmethanesulfonamide

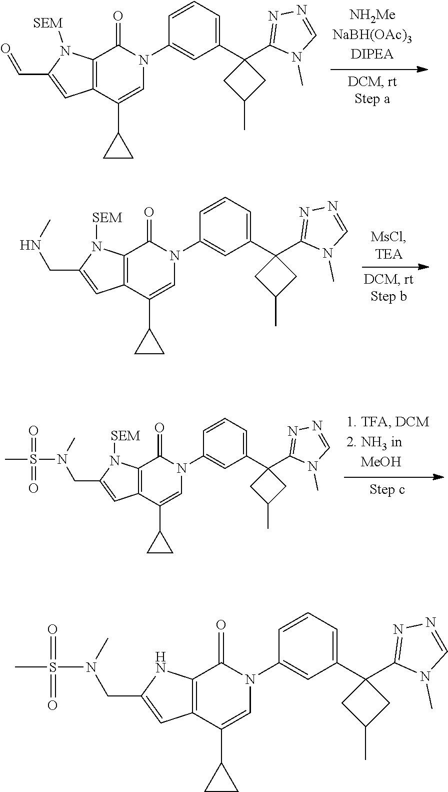 [SMILES: CC1CC(c2cccc(-n3cc(C4CC4)c4cc(C=O)n(COCC[Si](C)(C)C)c4c3=O)c2)(c2nncn2C)C1 ; "NH2MeNaBH(OAc)3DIPEA" ; CNCc1cc2c(C3CC3)cn(-c3cccc(C4(c5nncn5C)CC(C)C4)c3)c(=O)c2n1COCC[Si](C)(C)C ; CC1CC(c2cccc(-n3cc(C4CC4)c4cc(CN(C)S(C)(=O)=O)n(COCC[Si](C)(C)C)c4c3=O)c2)(c2nncn2C)C1 ; CC1CC(c2cccc(-n3cc(C4CC4)c4cc(CN(C)S(C)(=O)=O)[nH]c4c3=O)c2)(c2nncn2C)C1]

Step a: The product of step a 4-cyclopropyl-6-[3-[3,3-difluoro-1-(4-methyl-1,2,4-triazol-3-yl)cyclobutyl]phenyl]-2-[(3,3-difluoropyrrolidin-1-yl)methyl]-1-(2-trimethylsilylethoxymethyl)pyrrolo[2,3-c]pyridin-7-one was prepared in a similar fashion to that described for Example 39 from methylamine solution.

Step b: To the product of step a (50 mg, 0.08741 mmol, 1.0 equiv.) in DCM (3 mL, 0.03 M) was added MsCl (11 mg, 0.09615 mmol, 1.1 equiv.) and TEA (0.04 mL, 0.2622 mmol, 3.0 equiv.) and the mixture was stirred at rt for 2 h. The reaction was quenched with NaHCO₃ sat., the organic phase was separated, and the aqueous layer was extracted with EtOAc. The combined organic phase was dried over Na₂SO₄, concentrated and the crude residue was purified by column chromatography (SiO₂, MeOH in DCM, 0 to 15%) to give N-[[4-cyclopropyl-6-[3-[3-methyl-1-(4-methyl-1,2,4-triazol-3-yl)cyclobutyl]phenyl]-7-oxo-1-(2-trimethylsilylethoxymethyl)pyrrolo[2,3-c]pyridin-2-yl]methyl]-N-methylmethanesulfonamide.

Step c: To a solution of the crude product from step b in DCM (3 ml, 0.04 M) was add TFA (1 ml, excess). The resulting solution was stirred at rt for 1 h. The solvent was removed and NH₃ in MeOH (3 ml, excess, 7 N) was added and stirred for 30 min. The solvent was removed, and the resulting residue was further was purified by prep-HPLC to furnish the title compound. ¹H NMR (400 MHz, CDCl₃) δ 10.70 (s, 1H), 8.06 (s, 1H), 7.50-7.36 (m, 2H), 7.33-7.27 (m, 1H), 7.23 (d, J=1.4 Hz, 1H), 6.73 (d, J=1.3 Hz, 1H), 6.52 (d, J=2.2 Hz, 1H), 4.36 (s, 2H), 3.35 (s, 3H), 3.20 (td, J=8.5, 2.7 Hz, 2H), 2.75 (d, J=8.7 Hz, 6H), 2.58 (q, J=7.9 Hz, 1H), 2.32 (td, J=9.3, 2.7 Hz, 2H), 1.88 (dddd, J=9.2, 7.7, 5.6, 4.4 Hz, 1H), 1.13 (d, J=6.6 Hz, 3H), 0.96-0.82 (m, 2H), 0.72-0.59 (m, 2H). ESI MS [M+H]⁺ for C₂₇H₃₃N₆O₃S, calcd 521.6, found 521.1.

### Example 70: 2-({5-azaspiro[2.4]heptan-5-yl}methyl)-4-cyclopropyl-6-{3-[5-(4-methyl-4H-1,2,4-triazol-3-yl)spiro[2.3]hexan-5-yl]phenyl}-1H,6H,7H-pyrrolo[2,3-c]pyridin-7-one

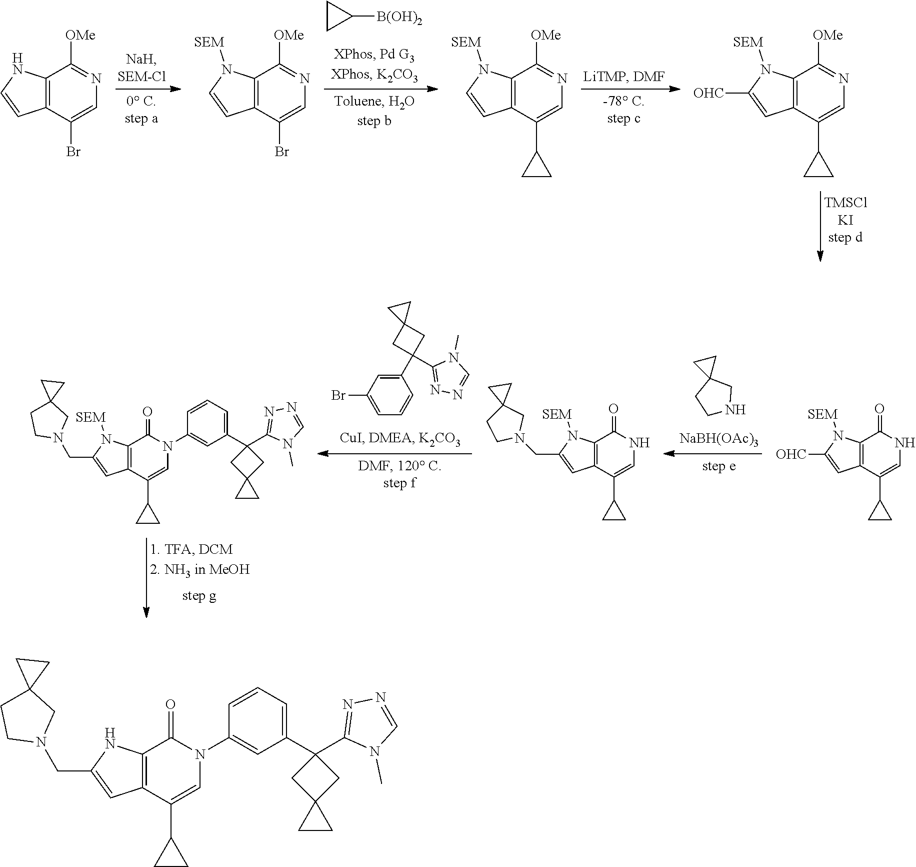

Step a: To a solution of 4-bromo-7-methoxy-1H-pyrrolo[2,3-c]pyridine (8.0, 35.24 mmol, 1.0 equiv.) in THE (100 ml, 0.3 M) was add NaH (2.54 g, 105.72 mmol, 3.0 equiv.) and SEMCl (6.46 g, 38.76 mmol, 1.1 equiv.) at 0° C. The resulting mixture was stirred at rt for 2 h. The reaction mixture was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexanes, 20 to 80%) to give 2-[(4-bromo-7-methoxypyrrolo[2,3-c]pyridin-1-yl)methoxy]ethyl-trimethylsilane.

Step b: The product of step a (6.60 g, 18.44 mmol, 1.0 equiv.), cyclopropylboronic acid (2.0 g, 23.04 mmol, 1.25 equiv.) and K₂CO₃ (7.60 g, 55.31 mmol, 3.0 equiv.) were dissolved in toluene/H₂O (60 mL/12 ml, 0.25 M). The mixture was purged for 2 mins under N₂. Then, Xphos Pd G3 (780 mg, 0.9218 mmol, 0.05 equiv.) and Xphos (703 mg, 1.4749 mmol, 0.08 equiv.) were added into the solution. The mixture was stirred at 90° C. for 12 h. After cooling down to rt, the reaction mixture was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexanes, 20 to 80%) to give 2-[(4-cyclopropyl-7-methoxypyrrolo[2,3-c]pyridin-1-yl)methoxy]ethyl-trimethylsilane.

Step c: To solution of 2,2,6,6-tetramethylpiperidine (1.53 ml, 8.9826 mmol, 1.6 equiv.) in THE (50 mL, 0.18 M) was added n-butyllithium solution (3.6 ml, 8.9826 mmol, 1.6 equiv., 2.5 M) slowly at −78° C. The resulting mixture was stirred at −78° C. for 5 min. To the resulting mixture was added the product of step b (1.7881 g, 5.6141 mmol, 1.0 equiv.) at −78° C. The resulting mixture was stirred at −78° C. for 1 h. Then, DMF (0.8 ml, 10.1054 mmol, 1.8 equiv.) was added to the mixture and stirred for 30 min at −78° C. The reaction was quenched with sat. aq. NH₄Cl solution, the organic phase was separated, and the aqueous layer was extracted with EtOAc. The combined organic phase was dried over Na₂SO₄ and concentrated to afford the crude residue. The crude product 2-[(4-cyclopropyl-7-methoxypyrrolo[2,3-c]pyridin-1-yl)methoxy]ethyl-trimethylsilane was used for the next step without purification.

Step d: To a solution of the product from step c (1.95 g, 5.6 mmol, 1.0 equiv.) and KI (1.50 g, 8.96 mmol, 1.6 equiv.) in CH₃CN (50 mL, 0.1 M) was added TMSCl (977 mg, 8.96 mmol, 1.6 equiv.) dropwise at rt. H₂O (0.1 ml) was added into the solution. The resulting mixture was stirred at rt for 12 h, then quenched with H₂O. The organic phase was separated, and the aqueous phase was extracted with EtOAc, the combined organic phase was then washed with brine, dried over Na₂SO₄ and concentrated and the crude residue was purified by column chromatography (SiO₂, MeOH in DCM, 0 to 10%) to give 4-cyclopropyl-7-oxo-1-(2-trimethylsilylethoxymethyl)-6H-pyrrolo[2,3-c]pyridine-2-carbaldehyde.

Step e: To the product of step d (128 mg, 0.3849 mmol, 1.0 equiv.) in DCM (5 mL, 0.08 M) was added 5-azaspiro[2.4]heptane (103 mg, 0.7680 mmol, 2.0 equiv.) and DIPEA (0.17 mL, 0.9623. mmol, 2.5 equiv.) and the mixture was stirred at rt for 10 mins. NaBH(OAc)₃ (214 mg, 0.9623 mmol, 2.5 equiv.) was added and the mixture was stirred at rt for 12 h. The reaction was quenched with NaHCO₃ sat., the organic phase was separated, and the aqueous layer was extracted with EtOAc. The combined organic phase was dried over Na₂SO₄, concentrated and the crude residue was purified by column chromatography (SiO₂, MeOH in DCM, 0 to 15%) to give 2-(5-azaspiro[2.4]heptan-5-ylmethyl)-4-cyclopropyl-1-(2-trimethylsilylethoxymethyl)-6H-pyrrolo[2,3-c]pyridin-7-one.

Step f: To a solution of the product from step e (50 mg, 0.1277 mmol, 1.0 equiv.) and 3-[5-(3-bromophenyl)spiro[2.3]hexan-5-yl]-4-methyl-1,2,4-triazole (28 mg, 0.0911 mmol, 1.0 equiv.) from Example 10, step e, in dioxane (5 mL, 0.025 M) was added CuI (24 mg, 0.1277 mmol, 1.0 equiv.), DMEA (45 mg, 0.5108 mmol, 4.0 equiv.) and K₂CO₃ (53 mg, 0.3821 mmol, 3.0 equiv.). The resulting solution was stirred at 110° C. for 2 h. The reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 20%) to give a crude mixture. The crude product 2-(5-azaspiro[2.4]heptan-5-ylmethyl)-4-cyclopropyl-6-[3-[5-(4-methyl-1,2,4-triazol-3-yl)spiro[2.3]hexan-5-yl]phenyl]-1-(2-trimethylsilylethoxymethyl)pyrrolo[2,3-c]pyridin-7-one was used for the next step without purification.

Step g: To a solution of the crude product from step f in DCM (3 ml, 0.04 M) was add TFA (1 ml, excess). The resulting solution was stirred at rt for 1 h. The solvent was removed and NH₃ in MeOH (3 ml, excess, 7 N) was added and stirred for 30 min. The solvent was removed, and the resulting residue was further was purified by prep-HPLC to furnish the title compound. ¹H NMR (400 MHz, CDCl₃) δ 11.57 (s, 1H), 8.08 (s, 1H), 7.54-7.42 (m, 2H), 7.31-7.27 (m, 1H), 7.19 (t, J=1.9 Hz, 1H), 6.73 (d, J=1.3 Hz, 1H), 6.57 (s, 1H), 4.25 (s, 2H), 3.30 (m, 6H), 2.98 (s, 2H), 2.86-2.71 (m, 2H), 1.99 (t, J=7.3 Hz, 2H), 1.88-1.80 (m, 1H), 0.92-0.81 (m, 2H), 0.69 (s, 5H), 0.63-0.51 (m, 6H). ESI MS [M+H]⁺ for C₃₂H₃₇N₆O, calcd 521.7, found 521.1.

### Example 71: 4-cyclopropyl-2-[[(2R)-2-methylmorpholin-4-yl]methyl]-6-[3-[5-(4-methyl-1,2,4-triazol-3-yl)spiro[2.3]hexan-5-yl]phenyl]-1H-pyrrolo[2,3-c]pyridin-7-one

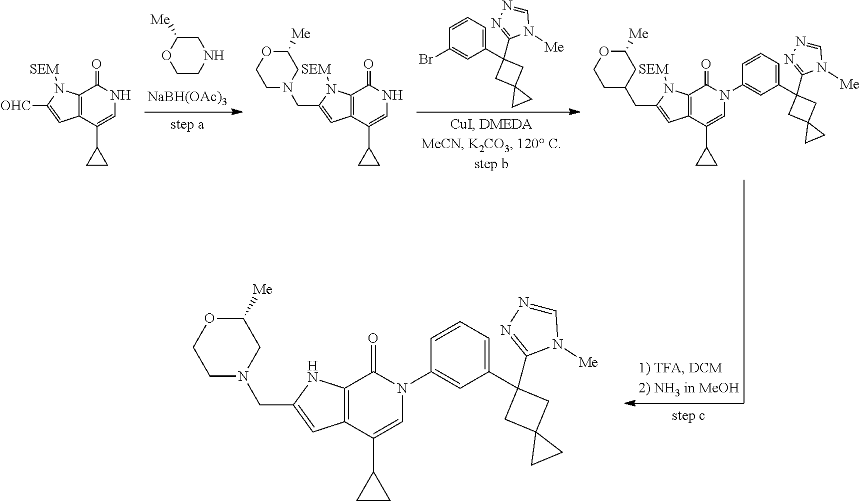

Step a: To a solution of 4-cyclopropyl-7-oxo-1-(2-trimethylsilylethoxymethyl)-6H-pyrrolo[2,3-c]pyridine-2-carbaldehyde (22.2 mg, 0.067 mmol) in 0.5 mL DCM, (2R)-2-methyl-morpholine (8.1 mg, 0.08 mmol) was added. The reaction was stirred at room temperature for 30 mins and NaBH(OAc)₃ (21 mg, 0.1 mmol) was added, and stirred for another 1 hour. Water was added and the solution was extracted by DCM. The combined organic phase was dried over Na₂SO₄, concentrated and the crude residue was purified by column chromatography (SiO₂, 0-20% MeOH/DCM) to give the desired product.

Step b: To a solution of the product from step a (52 mg, 0.12 mmol), 3-[5-(3-bromophenyl)spiro[2.3]hex-5-yl]-4-methyl-4H-1,2,4-triazole (59 mg, 0.19 mmol), and DMEDA (16 μL, 0.15 mmol) in 1.5 mL MeCN, K₂CO₃ (51.3 mg, 0.38 mmol) was added in one portion. Nitrogen was bubbled into the solution for 10 mins followed by adding CuI (24 mg, 0.12 mmol). The reaction was stirred in a sealed vial at 100° C. for 2 hours. After cooling to room temperature, saturated NH₄Cl was added, and the solution was extracted by DCM. The combined organic phase was dried over Na₂SO₄, concentrated and the crude residue was purified by column chromatography (SiO₂, 0-10% MeOH/DCM) to give the desired product. The product was used for the next step directly.

Step c: To a solution of the product from step b in 1 mL DCM, 1 mL TFA was added dropwise, and the reaction was stirred for 1 hour and concentrated under vacuum. The crude product was dissolved in 1 mL NH₃ in MeOH and stir for another 1 hour at room temperature before the crude product was concentrated and the crude residue was purified by prep-HPLC. ¹H NMR (400 MHz, CDCl₃) δ 9.80 (s, 1H), 8.06 (s, 1H), 7.54-7.39 (m, 2H), 7.31-7.26 (m, 2H), 6.74 (s, 1H), 6.42 (s, 1H), 3.85 (d, J=11.3 Hz, 1H), 3.76-3.56 (m, 4H), 3.35-3.22 (m, 5H), 2.79 (d, J=11.6 Hz, 2H), 2.70 (dd, J=16.9, 11.4 Hz, 2H), 2.23 (dd, J=12.4, 9.4 Hz, 2H), 1.89 (t, J=10.6 Hz, 2H), 1.13 (d, J=6.1 Hz, 3H), 0.86 (dd, J=8.0, 5.7 Hz, 2H), 0.64 (d, J=5.5 Hz, 2H), 0.57 (p, J=7.5, 6.7 Hz, 4H). ESI MS [M+H]⁺ for C₃₁H₃₆N₆O₂, calcd 525.3, found 525.4.

### Example 72: 4-cyclopropyl-6-{3-[5-(4-methyl-4H-1,2,4-triazol-3-yl)spiro[2.3]hexan-5-yl]phenyl}-2-{[(2S)-2-(propan-2-yl)piperazin-1-yl]methyl}-1H,6H,7H-pyrrolo[2,3-c]pyridin-7-one

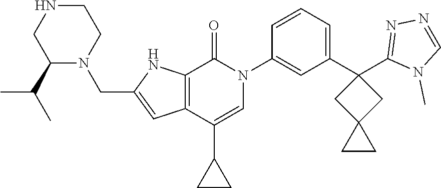 [SMILES: CC(C)[C@H]1CNCCN1Cc1cc2c(C3CC3)cn(-c3cccc(C4(c5nncn5C)CC5(CC5)C4)c3)c(=O)c2[nH]1]

The title compound was prepared in a similar fashion to that described for Example 71 from (S)-1-Boc-3-isopropylpiperazine and 3-[5-(3-bromophenyl)spiro[2.3]hexan-5-yl]-4-methyl-1,2,4-triazole. ¹HNMR (400 MHz, CDCl₃) δ 10.10 (s, 1H), 8.09 (s, 1H), 7.57-7.33 (m, 2H), 7.23 (dt, J=3.9, 1.7 Hz, 2H), 6.73 (d, J=1.2 Hz, 1H), 6.41 (s, 1H), 4.21 (d, J=14.4 Hz, 1H), 3.43 (d, J=14.3 Hz, 1H), 3.32 (s, 4H), 3.28 (d, J=11.1 Hz, 2H), 3.20 (d, J=12.2 Hz, 1H), 3.09 (d, J=12.4 Hz, 1H), 2.90 (dt, J=32.0, 12.4 Hz, 2H), 2.49 (dd, J=26.8, 12.4 Hz, 3H), 2.33 (dd, J=12.0, 6.4 Hz, 1H), 1.87 (tt, J=9.1, 4.5 Hz, 1H), 0.97 (dd, J=10.8, 6.8 Hz, 7H), 0.89-0.82 (m, 2H), 0.69-0.48 (m, 6H). ESI MS [M+H]⁺ for C₃₃H₄₂N₇O, calcd 552.8, found 552.1.

### Example 73: 4-cyclopropyl-2-{[(2S)-4-methyl-2-(propan-2-yl)piperazin-1-yl]methyl}-6-{3-[5-(4-methyl-4H-1,2,4-triazol-3-yl)spiro[2.3]hexan-5-yl]phenyl}-1H,6H,7H-pyrrolo[2,3-c]pyridin-7-one

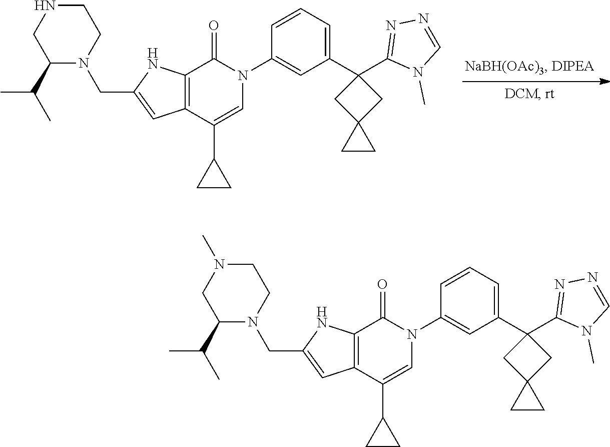 [SMILES: CC(C)[C@H]1CNCCN1Cc1cc2c(C3CC3)cn(-c3cccc(C4(c5nncn5C)CC5(CC5)C4)c3)c(=O)c2[nH]1 ; CC(C)[C@H]1CN(C)CCN1Cc1cc2c(C3CC3)cn(-c3cccc(C4(c5nncn5C)CC5(CC5)C4)c3)c(=O)c2[nH]1]

To a solution of 4-cyclopropyl-6-{3-[5-(4-methyl-4H-1,2,4-triazol-3-yl)spiro[2.3]hexan-5-yl]phenyl}-2-{[(2S)-2-(propan-2-yl)piperazin-1-yl]methyl}-1H,6H,7H-pyrrolo[2,3-c]pyridin-7-one (Example 73) (71 mg, 0.1286 mmol, 1.0 equiv.) in DCM (3 mL, 0.04 M) was added DIPEA (0.06 ml, 0.3215 mmol, 2.5 equiv.) and NaBH(OAc)₃ (72 mg, 0.1286 mmol, 1.0 equiv.). The resulting solution was stirred for 2 h. The reaction was quenched with NaHCO₃ sat., the organic phase was separated, and the aqueous layer was extracted with EtOAc. The combined organic phase was dried over Na₂SO₄, concentrated and the crude residue was purified by column chromatography (SiO₂, MeOH in DCM, 0 to 15%) to give the crude mixture. The crude mixture was further was purified by prep-HPLC to furnish the title compound. ¹H NMR (400 MHz, CDCl₃) δ 9.63 (s, 1H), 8.06 (s, 1H), 7.49 (t, J=7.8 Hz, 1H), 7.43 (dt, J=8.1, 1.4 Hz, 1H), 7.28 (t, J=1.9 Hz, 1H), 7.24 (d, J=1.6 Hz, OH), 6.74 (d, J=1.2 Hz, 1H), 6.41 (d, J=2.0 Hz, 1H), 4.23 (d, J=14.3 Hz, 1H), 3.42-3.17 (m, 7H), 3.00 (d, J=11.2 Hz, 1H), 2.91 (d, J=11.1 Hz, 1H), 2.79 (dd, J=10.7, 3.9 Hz, 3H), 2.56-2.48 (m, 1H), 2.45 (s, 3H), 2.39-2.13 (m, 3H), 1.92-1.81 (m, 1H), 1.00 (d, J=6.9 Hz, 4H), 0.95 (d, J=6.9 Hz, 3H), 0.91-0.84 (m, 2H), 0.72-0.51 (m, 7H). ESI MS [M+H]⁺ for C₃₄H₄₄N₇O, calcd 566.8, found 566.1.

### Example 74: 4-cyclopropyl-2-[[(3S)-3-methylpiperidin-1-yl]methyl]-6-[3-[5-(4-methyl-1,2,4-triazol-3-yl)spiro[2.3]hexan-5-yl]phenyl]-1H-pyrrolo[2,3-c]pyridin-7-one

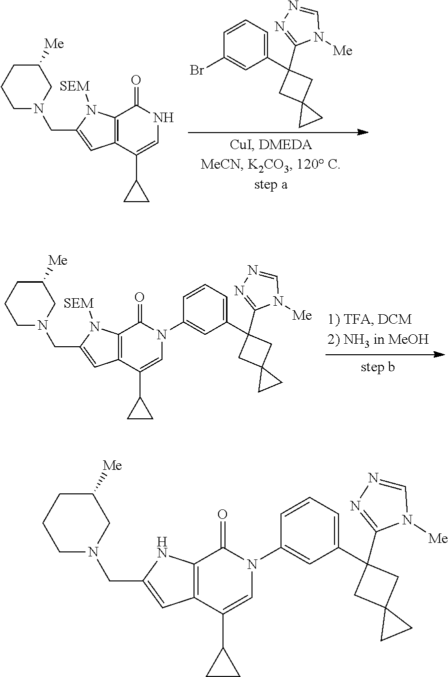 [SMILES: C[C@H]1CCCN(Cc2cc3c(C4CC4)c[nH]c(=O)c3n2COCC[Si](C)(C)C)C1 ; Cn1cnnc1C1(c2cccc(Br)c2)CC2(CC2)C1 ; C[C@H]1CCCN(Cc2cc3c(C4CC4)cn(-c4cccc(C5(c6nncn6C)CC6(CC6)C5)c4)c(=O)c3n2COCC[Si](C)(C)C)C1 ; C[C@H]1CCCN(Cc2cc3c(C4CC4)cn(-c4cccc(C5(c6nncn6C)CC6(CC6)C5)c4)c(=O)c3[nH]2)C1]

Step a: To a solution of 4-cyclopropyl-2-[[(3S)-3-methylpiperidin-1-yl]methyl]-1-(2-trimethylsilylethoxymethyl)-6H-pyrrolo[2,3-c]pyridin-7-one (62.3 mg, 0.15 mmol), 3-[5-(3-bromophenyl)spiro[2.3]hex-5-yl]-4-methyl-4H-1,2,4-triazole (71.3 mg, 0.22 mmol), and DMEDA (20 μL, 0.18 mmol) in 1 mL MeCN, and K₂CO₃ (62 mg, 0.45 mmol) was added in one portion. Nitrogen was bubbled into the solution for 10 mins followed by adding CuI (28.5 mg, 0.15 mmol). The reaction was stirred in sealed vial at 100° C. for 2 hours. After cooling to room temperature, saturated NH₄Cl was added, and the solution was extracted by DCM. The combined organic phase was dried over Na₂SO₄, concentrated and the crude residue was purified by column chromatography (SiO₂, 0-10% MeOH/DCM) to give the desired product. The product was used for the next step directly.

Step b: To a solution of the product from step a in 2 mL DCM, 2 mL TFA were added dropwise, and the reaction was stirred for 1 hour and concentrated under vacuum. The crude product was dissolved in 2 mL NH₃ in MeOH and stir for another 1 hour at room temperature before the crude product was concentrated and the crude residue was purified by prep-HPLC. ¹H NMR (400 MHz, CDCl₃) δ 10.98 (s, 1H), 8.07 (s, 1H), 7.48 (t, J=7.5 Hz, 1H), 7.42 (d, J=7.1 Hz, 2H), 7.28 (s, 1H), 6.73 (s, 1H), 6.46 (s, 1H), 3.86 (s, 2H), 3.32 (s, 3H), 3.29 (d, J=12.0 Hz, 2H), 3.01 (dd, J=26.3, 9.4 Hz, 2H), 2.78 (d, J=11.3 Hz, 2H), 2.14 (td, J=11.2, 3.8 Hz, 1H), 1.94-1.60 (m, 6H), 0.96-0.81 (m, 6H), 0.66-0.51 (m, J=24.4, 11.8, 6.4 Hz, 6H). ESI MS [M+H]⁺ for C₃₂H₃₈N₆O₃, calcd 523.3, found 523.4.

### Example 75: 4-cyclopropyl-2-[[(3S)-3-methylpiperidin-1-yl]methyl]-6-[3-[3-[(4-methyl-1,2,4-triazol-3-yl)methyl]oxetan-3-yl]phenyl]-1H-pyrrolo[2,3-c]pyridin-7-one

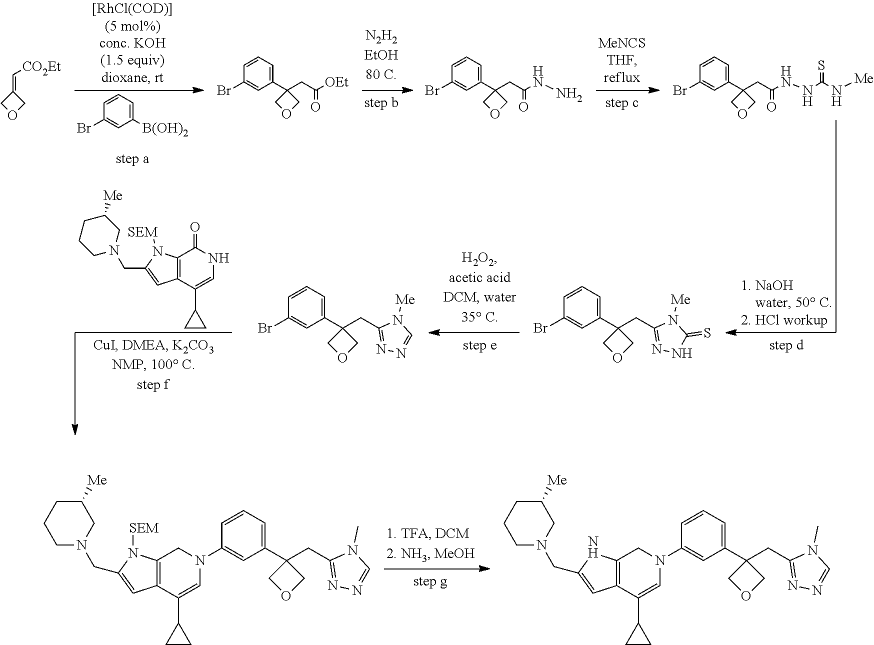

Step a: To a 500-mL 3-neck round bottom flask was added dioxane (18.0 mL) followed by saturated KOH (aq.) (3.6 mL) and ethyl 2-(oxetan-3-ylidene)acetate (4.0 g, 28.0 mmol). A solution of (3-bromophenyl)boronic acid (7.3 g, 36.4 mmol) in dioxane (20.0 mL) was added dropwise. The internal temperature was kept between 15-20° C. by cooling with an iced water bath. After stirring overnight, the precipitate was removed by filtration. The filtrate was concentrated to dryness. Purification by column chromatography (SiO₂, 0-45% EtOAc/hexanes) afforded the desired product.

Step b: The product from step a (7.3 g, 24.4 mmol) was dissolved in ethanol (40.6 mL, 0.6 M), hydrazine hydrate was added (3.8 mL, 61.0 mmol, 2.5 equiv.), and the reaction was heated to 80° C. for 16 hours. After cooling to room temperature, the reaction was concentrated to dryness and diluted with water and ethyl acetate. The organic phase was separated, and the aqueous layer was extracted with EtOAc. The organic layers were combined, dried over MgSO₄, and concentrated to afford the crude product.

Step c: To a suspension of the crude product in THF (32.7 mL) was added MeNCS (3.8 g, 52.3 mmol). The resulting mixture was stirred and refluxed for 1 hour. The reaction was concentrated onto Celite® and purified directly by flash chromatography (SiO₂, 0-50% MeOH/DCM) to furnish the product.

Step d: A suspension of the product from step c (3.1 g, 8.6 mmol) in 1 M NaOH (34.5 mL) was heated at 50° C. for one hour. After the completion of the reaction, conc. HCl was added to adjust the pH to 1. The resulting white precipite was collected by filtration to yield the desired product.

Step e: To a solution of the product from step d (3.2 g, 9.4 mmol) in dichloromethane (40 mL) was added a solution of H₂O₂ (30% purity, 3.2 mL) in AcOH (0.8 mL). The reaction mixture was stirred at 35° C. for 16 hours. After the reaction was complete, sat. Na₂CO₃ (aq) was added carefully until the pH=10. The mixture was then extracted with dichloromethane. The combined organic phase was dried over MgSO₄, and concentrated to give the desired product.

Step f: To a solution of the product from step e (166.4 mg, 0.54 mmol) and 4-cyclopropyl-2-[[(3S)-3-methylpiperidin-1-yl]methyl]-1-(2-trimethylsilylethoxymethyl)-6H-pyrrolo[2,3-c]pyridin-7-one (150 mg, 0.49 mmol) in NMP (4.9 mL, 0.1 M) was added CuI (93.3 mg, 0.49 mmol), DMEA (86.4 mg, 0.98 mmol) and K₂CO₃ (138.2 mg, 1.47 mmol). The resulting solution was stirred at 100° C. for 2 h. The reaction was quenched with H₂O and extracted with EtOAc. The combined organic phase was dried over MgSO₄, concentrated and the crude residue was purified by flash column chromatography (SiO₂, MeOH in DCM, 0-10% MeOH/DCM) to furnish the title compound.

Step g: To a solution of the product from step f (120.0 mg, 0.226 mmol) in dichloromethane (1.1 mL) was added TFA (1.1 mL) and stirred for 1.5 hours at room temperature, then concentrated to dryness under reduced pressure. To this crude residue was added 7.0 M NH₃ in methanol (1.0 mL) and the mixture was stirred for 1.5 hours. The reaction mixture was concentrated, and the crude product was purified by prep-HPLC (20% to 90% MeCN/water, 0.1% TFA) to afford the title compound. ¹H NMR (400 MHz, CDCl₃) δ 9.86 (s, 1H), 7.86 (s, 1H), 7.32 (t, J=7.8 Hz, 1H), 7.19 (d, J=8.2 Hz, 1H), 6.76 (s, 1H), 6.71 (d, J=7.8 Hz, 1H), 6.59 (s, 1H), 6.35 (s, 1H), 5.09 (d, J=6.1 Hz, 2H), 4.99 (d, J=6.1 Hz, 2H), 3.52 (s, 2H), 3.44 (s, 2H), 2.81 (s, 3H), 1.94 (d, J=13.5 Hz, 1H), 1.82 (td, J=8.4, 4.3 Hz, 1H), 1.64 (dd, J=28.5, 13.4 Hz, 5H), 1.22 (s, 2H), 0.81 (t, J=8.1 Hz, 6H), 0.59 (d, J=5.3 Hz, 2H).ESI MS [M+H]⁺ for C₃₀H₃₇N₆₀₂, calcd 513.3, found 513.3.

### Example 76: 2-({5-azaspiro[2.4]heptan-5-yl}methyl)-4-cyclopropyl-6-(3-{3-[(4-methyl-4H-1,2,4-triazol-3-yl)methyl]oxetan-3-yl}phenyl)-1H,6H,7H-pyrrolo[2,3-c]pyridin-7-one

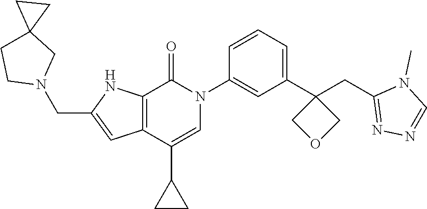 [SMILES: Cn1cnnc1CC1(c2cccc(-n3cc(C4CC4)c4cc(CN5CCC6(CC6)C5)[nH]c4c3=O)c2)COC1]

The title compound was prepared in a similar fashion to that described for Example 75. ¹H NMR (400 MHz, CDCl₃) δ 11.94 (s, 1H), 7.94 (s, 1H), 7.38 (t, J=7.8 Hz, 1H), 7.20 (ddd, J=7.9, 2.1, 1.0 Hz, 1H), 6.89 (dt, J=7.8, 1.3 Hz, 1H), 6.63 (dd, J=3.7, 1.6 Hz, 2H), 6.58 (s, 1H), 5.11 (d, J=6.1 Hz, 2H), 5.01 (d, J=6.2 Hz, 2H), 4.41 (s, 2H), 3.54 (m, 3H), 3.15 (s, 2H), 2.89 (s, 3H), 2.07 (t, J=7.3 Hz, 2H), 1.88-1.79 (m, 2H), 0.91-0.81 (m, 2H), 0.76 (s, 4H), 0.65-0.53 (m, 2H). ESI MS [M+H]⁺ for C₃₀H₃₄N₆O₂, calcd 511.7, found 511.1.

### Example 77: 4-cyclopropyl-6-{3-[5-(4-methyl-4H-1,2,4-triazol-3-yl)spiro[2.3]hexan-5-yl]phenyl}-2-{[(1-methylcyclobutyl)amino]methyl}-1H,6H,7H-pyrrolo[2,3-c]pyridin-7-one

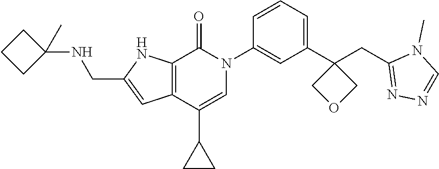 [SMILES: Cn1cnnc1CC1(c2cccc(-n3cc(C4CC4)c4cc(CNC5(C)CCC5)[nH]c4c3=O)c2)COC1]

The title compound was prepared in a similar fashion to that described for Example 75 from 1-methylcyclobutanamine hydrochloride. ¹H NMR (400 MHz, CDCl₃δ 8.49 (s, 1H), 8.07 (s, 1H), 7.52 (t, J=7.8 Hz, 1H), 7.48-7.43 (m, 1H), 7.30-7.26 (m, 1H), 7.21 (t, J=2.0 Hz, 1H), 6.61 (d, J=1.2 Hz, 1H), 6.33 (s, 1H), 4.00 (s, 2H), 3.28 (d, J=11.5 Hz, 5H), 2.78 (d, J=12.6 Hz, 2H), 2.29 (q, J=9.9, 9.3 Hz, 2H), 1.92-1.83 (m, 1H), 1.81-1.72 (m, 2H), 1.45 (s, 3H), 0.89-0.79 (m, 2H), 0.57 (dtd, J=9.3, 6.4, 5.8, 3.7 Hz, 6H). ESI MS [M+H]⁺ for C₃₁H₃₇N₆O, calcd 509.7, found 509.1.

### Example 78: 4-cyclopropyl-6-[3-[3,3-difluoro-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

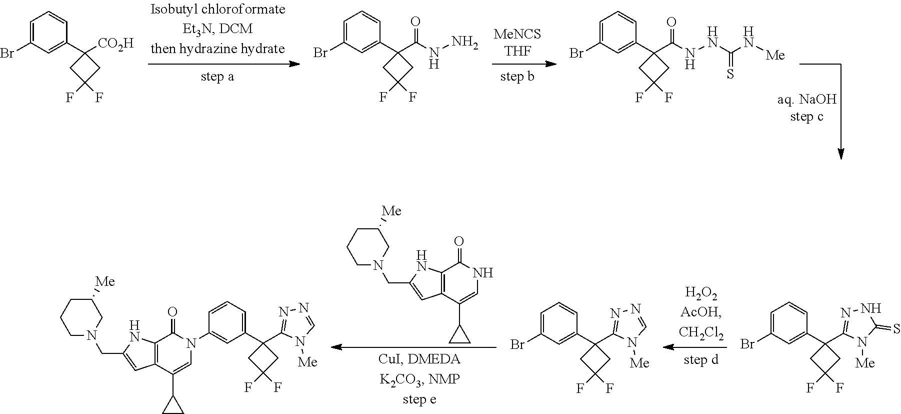

Step a: Triethylamine (1.1 mL, 7.9 mmol) was added to a solution of 1-(3-bromophenyl)-3,3-difluorocyclobutane-1-carboxylic acid (1.0 g, 3.4 mmol) in dichloromethane (35.0 mL). The resulting solution was placed in 100 mL single neck round bottom flask equipped with magnetic stirring bar and drying tube. The reaction was cooled to 0° C. before isobutyl chloroformate (0.49 mL, 3.8 mmol) was added dropwise over 1 min. The reaction mixture was allowed to warm and stirred for 1 h at 23° C. The brownish solution was cooled back to 0° C. and hydrazine hydrate (0.8 mL, 13.8 mmol, 55% purity) was added in one portion. The reaction mixture was allowed to warm to 23° C. and stirred for 20 min. Then it was diluted with dichloromethane (20.0 mL) and poured into aq. sat. sodium bicarbonate solution (20.0 mL). The organic phase was separated, and the aqueous phase was additionally extracted with dichloromethane (2×15.0 mL). The combined organic extract was dried over sodium sulfate, and all volatiles were removed under reduced pressure. The crude product obtained upon concentration was used directly for step b.

Step b: The acylhydrazine obtained in step a (3.4 mmol) was dissolved in THF and methyl isothiocyanate (0.7 mL, 10.3 mmol) was added. The resulting mixture was maintained at 65° C. for 1 h. Then it was cooled to room temperature, and all volatiles were removed under reduced pressure. The crude product was purified by column chromatography (SiO₂, dichloromethane/EtOAc gradient) to produce the desired thiosemicarbazide.

Step c: Thiosemicarbazide (1.2 g, 3.3 mmol) from step b was dissolved in 16 ml of 1M aq. NaOH and the resulting clear solution was stirred at 70° C. for 30 min. The reaction mixture was cooled to room temperature, acidified with 1M aq. hydrochloric acid until pH˜3, and the formed product was extracted with EtOAc (3×25.0 mL). The combined organic extract was dried over sodium sulfate and concentrated to dryness to produce the cyclized product that was directly used for the next step.

Step d: The cyclized product of step c (3.3 mmol) was dissolved in a mixture of dichloromethane (14.5 mL) and acetic acid (1.8 mL) and placed in a 100 mL single neck round bottom flask equipped with magnetic stirring bar. The reaction mixture was cooled to 0° C., and hydrogen peroxide (0.9 mL, 8.5 mmol, 30% aq. solution) was added. The resulting biphasic mixture was stirred for 20 min, then cooling bath was removed, and the reaction mixture was maintained at room temperature for an additional 1 h. The mixture was diluted with dichloromethane (30.0 mL) and 1 M aq. NaOH (30.0 mL). The organic phase was separated, and the aqueous phase was additionally extracted with dichloromethane (2×20.0 mL). The combined organic extract was washed with 1M aq. NaOH (40.0 mL), water (40.0 mL) and brine (40.0 mL), dried over sodium sulfate and concentrated to dryness under reduced pressure to produce the desired 1,3,4-triazole.

Step e: The bromide obtained in step d (138.0 mg, 0.42 mmol) was combined with 4-cyclopropyl-2-[[(3S)-3-methylpiperidin-1-yl]methyl]-1,6-dihydropyrrolo[2,3-c]pyridin-7-one (100 mg, 0.35 mmol), 1,2-dimethylethylenediamine (70 uL, 0.7 mmol) and potassium carbonate (145.0 mg, 1.1 mmol) in dry NMP (3.5 mL). This mixture was placed in 40 mL screw cap vial equipped with magnetic stirring bar, degassed under vacuum, and backfilled with nitrogen (repeated 3 times). Copper (I) iodide (67.0 mg, 0.35 mmol) was added, the mixture was degassed again using previous method and heated at 100° C. for 1 h. Then the mixture was cooled to ambient temperature, diluted with aq. sat. NH₄Cl (10.0 mL), EtOAc (25.0 mL) and water (20.0 mL). The organic phase was separated, and the aqueous phase was additionally extracted with ethyl acetate (2×20.0 mL). The combined organic extract was washed with water (3×30.0 mL) dried over sodium sulfate, filtered, and concentrated to dryness. The crude product was purified by reversed phase column chromatography (C18-modified SiO₂, water/CH₃CN gradient containing with 0.1% formic acid) to produce the desired coupling product. ¹H NMR (400 MHz, CDCl₃) δ 9.56 (s, 1H), 8.05 (s, 1H), 7.49 (t, J=7.8 Hz, 1H), 7.36 (s, 1H), 7.30 (d, J=7.8 Hz, 2H), 6.72 (s, 1H), 6.38 (s, 1H), 3.82-3.68 (m, 2H), 3.65-3.55 (m, 2H), 3.41-3.26 (m, 5H), 2.91-2.63 (m, 2H), 2.01-1.83 (m, 2H), 1.75-1.46 (m, 5H), 0.96-0.77 (m, 6H), 0.64 (d, J=5.4 Hz, 2H).

### Example 79: 4-cyclopropyl-6-{3-[3,3-difluoro-1-(4-methyl-4H-1,2,4-triazol-3-yl)cyclobutyl]phenyl}-2-[(3,3-difluoropyrrolidin-1-yl)methyl]-1H,6H,7H-pyrrolo[2,3-c]pyridin-7-one

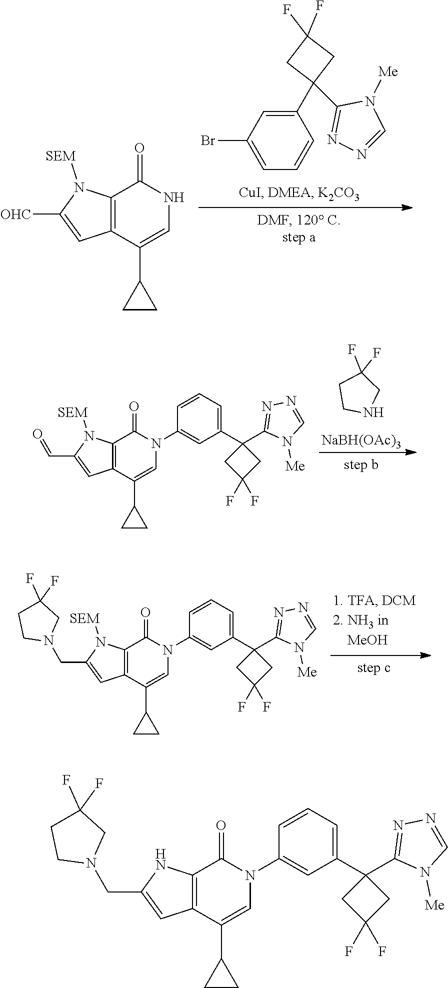

Step a: To a solution of 4-cyclopropyl-7-oxo-1-(2-trimethylsilylethoxymethyl)-6H-pyrrolo[2,3-c]pyridine-2-carbaldehyde (470 mg, 1.4184 mmol, 1.0 equiv.) and 3-[1-(3-bromophenyl)-3,3-difluorocyclobutyl]-4-methyl-1,2,4-triazole (700 mg, 2.1276 mmol, 1.0 equiv in dioxane (15 mL, 0.1M) was added CuI (270 mg, 1.4184 mmol, 1.0 equiv.), DMEA (500 mg, 5.6736 mmol, 4.0 equiv.) and K₂CO₃ (590 mg, 4.2552 mmol, 3.0 equiv.). The resulting solution was stirred at 110° C. for 2 h. The reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 20%) to give 4-cyclopropyl-6-[3-[3,3-difluoro-1-(4-methyl-1,2,4-triazol-3-yl)cyclobutyl]phenyl]-7-oxo-1-(2-trimethylsilylethoxymethyl)pyrrolo[2,3-c]pyridine-2-carbaldehyde.

Step b: To the product of step a (36 mg, 0.06207 mmol, 1.0 equiv.) in DCM (5 mL, 0.02 M) was added 3,3-difluoropyrrolidine hydrochloride (14 mg, 0.1241 mmol, 2.0 equiv.) and DIPEA (0.03 mL, 0.1552 mmol, 2.5 equiv.) and the mixture was stirred at rt for 10 mins. NaBH(OAc)₃ (35 mg, 0.1552 mmol, 2.5 equiv.) was added and the mixture was stirred at rt for 12 h. The reaction was quenched with NaHCO₃ sat., the organic phase was separated, and the aqueous layer was extracted with EtOAc. The combined organic phase was dried over Na₂SO₄, concentrated and the crude residue was purified by column chromatography (SiO₂, MeOH in DCM, 0 to 15%) to give 4-cyclopropyl-6-[3-[3,3-difluoro-1-(4-methyl-1,2,4-triazol-3-yl)cyclobutyl]phenyl]-2-[(3,3-difluoropyrrolidin-1-yl)methyl]-1-(2-trimethylsilylethoxymethyl)pyrrolo[2,3-c]pyridin-7-one.

Step c: To a solution of the crude product from step b in DCM (3 ml, 0.04 M) was add TFA (1 ml, excess). The resulting solution was stirred at rt for 1 h. The solvent was removed and NH₃ in MeOH (3 ml, excess, 7 N) was added and stirred for 30 min. The solvent was removed, and the resulting residue was further was purified by prep-HPLC to furnish the title compound. ¹H NMR (400 MHz, CDCl₃) δ 10.32 (s, 1H), 8.09 (s, 1H), 7.52 (t, J=8.0 Hz, 1H), 7.39-7.34 (m, 1H), 7.30 (dd, J=7.4, 1.2 Hz, 2H), 6.74 (d, J=1.3 Hz, 1H), 6.48 (d, J=2.1 Hz, 1H), 3.90 (s, 2H), 3.76 (q, J=13.3 Hz, 2H), 3.37 (s, 5H), 3.06 (t, J=12.9 Hz, 2H), 2.91 (t, J=7.0 Hz, 2H), 2.34 (dq, J=14.3, 7.2 Hz, 2H), 1.88 (dddd, J=9.7, 6.5, 5.3, 2.6 Hz, 1H), 0.95-0.82 (m, 2H), 0.69-0.57 (m, 2H). ESI MS [M+H]⁺ for C₂₈H₂₉F₄N₆O, calcd 541.6, found 541.1.

### Example 80: 4-cyclopropyl-6-{3-[3,3-difluoro-1-(4-methyl-4H-1,2,4-triazol-3-yl)cyclobutyl]phenyl}-2-{[(3,3-difluorocyclopentyl)amino]methyl}-1H,6H,7H-pyrrolo[2,3-c]pyridin-7-one

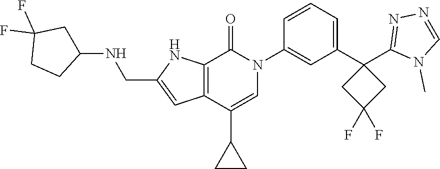 [SMILES: Cn1cnnc1C1(c2cccc(-n3cc(C4CC4)c4cc(CNC5CCC(F)(F)C5)[nH]c4c3=O)c2)CC(F)(F)C1]

The title compound was prepared in a similar fashion to that described for Example 79 from 3,3-difluorocyclopentanamine and 3-[1-(3-bromophenyl)-3,3-difluorocyclobutyl]-4-methyl-1,2,4-triazole. ¹H NMR (400 MHz, CDCl₃) δ 8.09 (s, 1H), 7.70-7.50 (m, 1H), 7.45 (d, J=8.0 Hz, 1H), 7.28 (s, 1H), 7.22 (s, 1H), 6.59 (s, 1H), 6.34 (s, 1H), 4.24 (d, J=13.8 Hz, 1H), 4.07 (d, J=13.6 Hz, 1H), 3.78 (d, J=14.0 Hz, 2H), 3.62 (s, 1H), 3.38 (s, 4H), 2.40 (s, 1H), 2.25 (s, 3H), 1.73 (m, 2H), 1.26 (m, 1H), 0.93-0.79 (m, 2H), 0.55 (q, J=5.1 Hz, 2H). ESI MS [M+H]⁺ for C₂₉H₃₁F₄N₆O, calcd 555.6, found 555.1.

### Example 81: 4-cyclopropyl-6-{3-[3,3-difluoro-1-(4-methyl-4H-1,2,4-triazol-3-yl)cyclobutyl]phenyl}-2-{[(3,3-difluoro-1-methylcyclobutyl)amino]methyl}-1H,6H,7H-pyrrolo[2,3-c]pyridin-7-one

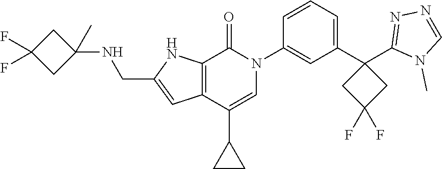 [SMILES: Cn1cnnc1C1(c2cccc(-n3cc(C4CC4)c4cc(CNC5(C)CC(F)(F)C5)[nH]c4c3=O)c2)CC(F)(F)C1]

The title compound was prepared in a similar fashion to that described for Example 79 from 3,3-difluoro-1-methylcyclobutanamine hydrochloride and 3-[1-(3-bromophenyl)-3,3-difluorocyclobutyl]-4-methyl-1,2,4-triazole. ¹H NMR (400 MHz, CDCl₃) δ 8.10 (s, 1H), 7.68-7.52 (m, 1H), 7.42 (d, J=8.0 Hz, 1H), 7.36 (d, J=7.8 Hz, 1H), 7.13 (s, 1H), 6.50 (s, 1H), 6.23 (s, 1H), 4.03 (s, 2H), 3.78 (q, J=13.2 Hz, 2H), 3.43-3.34 (m, 2H), 3.31 (s, 3H), 3.24-3.05 (m, 2H), 2.51 (d, J=13.9 Hz, 2H), 1.88-1.67 (m, 4H), 0.86 (d, J=8.4 Hz, 2H), 0.52 (d, J=5.4 Hz, 2H).

ESI MS [M+H]⁺ for C₂₉H₃₁F₄N₆O, calcd 555.6, found 555.1.

### Example 82: 4-cyclopropyl-6-{3-[3,3-difluoro-1-(4-methyl-4H-1,2,4-triazol-3-yl)cyclobutyl]phenyl}-2-{[(2R)-2-(hydroxymethyl)pyrrolidin-1-yl]methyl}-1H,6H,7H-pyrrolo[2,3-c]pyridin-7-one

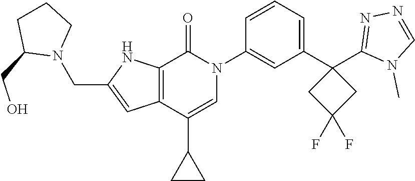 [SMILES: Cn1cnnc1C1(c2cccc(-n3cc(C4CC4)c4cc(CN5CCC[C@@H]5CO)[nH]c4c3=O)c2)CC(F)(F)C1]

The title compound was prepared in a similar fashion to that described for Example 79 from (−)-2-pyrrolidinemethanol and 3-[1-(3-bromophenyl)-3,3-difluorocyclobutyl]-4-methyl-1,2,4-triazole. ¹H NMR (400 MHz, CDCl₃) δ 11.67 (s, 1H), 8.11 (s, 1H), 7.51 (m, 2H), 7.37 (d, J=7.9 Hz, 1H), 7.31-7.27 (m, 1H), 6.73 (d, J=1.2 Hz, 1H), 6.67 (s, 1H), 4.68 (d, J=13.7 Hz, 1H), 4.38 (d, J=13.8 Hz, 1H), 4.00-3.81 (m, 2H), 3.75 (q, J=13.0 Hz, 2H), 3.56 (s, 2H), 3.37 (s, 5H), 3.07 (s, 1H), 2.22-1.96 (m, 5H), 1.85 (tt, J=9.1, 4.7 Hz, 1H), 0.95-0.81 (m, 2H), 0.70-0.56 (m, 2H). ESI MS [M+H]⁺ for C₂₉H₃₃F₂N₆O₂, calcd 535.6, found 535.1.

### Example 83: 4-cyclopropyl-6-{3-[3,3-difluoro-1-(4-methyl-4H-1,2,4-triazol-3-yl)cyclobutyl]phenyl}-2-({[(1-hydroxycyclobutyl)methyl]amino}methyl)-1H,6H,7H-pyrrolo[2,3-c]pyridin-7-one

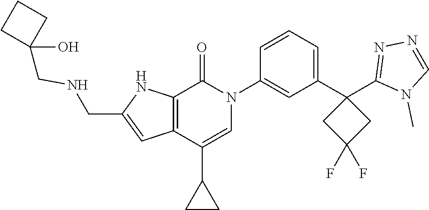 [SMILES: Cn1cnnc1C1(c2cccc(-n3cc(C4CC4)c4cc(CNCC5(O)CCC5)[nH]c4c3=O)c2)CC(F)(F)C1]

The title compound was prepared in a similar fashion to that described for Example 79 from 1-(aminomethyl)cyclobutanol and 3-[1-(3-bromophenyl)-3,3-difluorocyclobutyl]-4-methyl-1,2,4-triazole. ¹H NMR (400 MHz, CDCl₃) δ 8.13 (s, 1H), 7.54 (t, J=7.8 Hz, 1H), 7.39 (dd, J=7.9, 1.6 Hz, 1H), 7.28 (t, J=2.0 Hz, 1H), 7.25-7.22 (m, 1H), 6.65 (d, J=1.2 Hz, 1H), 6.62 (s, 1H), 4.44 (s, 2H), 3.75 (q, J=13.1 Hz, 2H), 3.36 (m, 5H), 3.15 (s, 2H), 2.10 (dd, J=21.9, 11.0 Hz, 4H), 1.80 (tt, J=8.6, 4.5 Hz, 1H), 1.70 (q, J=10.2 Hz, 1H), 1.57-1.43 (m, 1H), 0.93-0.81 (m, 2H), 0.64-0.51 (m, 2H). ESI MS [M+H]⁺ for C₂₉H₃₃F₂N₆O₂, calcd 535.6, found 535.1.

### Example 84: 4-cyclopropyl-6-{3-[3,3-difluoro-1-(4-methyl-4H-1,2,4-triazol-3-yl)cyclobutyl]phenyl}-2-({[1-(1-hydroxyethyl)cyclopropyl]amino}methyl)-1H,6H,7H-pyrrolo[2,3-c]pyridin-7-one

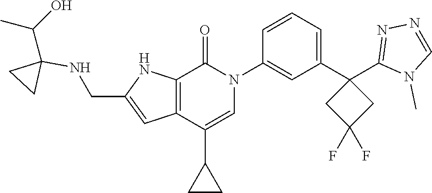 [SMILES: CC(O)C1(NCc2cc3c(C4CC4)cn(-c4cccc(C5(c6nncn6C)CC(F)(F)C5)c4)c(=O)c3[nH]2)CC1]

The title compound was prepared in a similar fashion to that described for Example 79 from 1-amino-α-methylcyclopropanemethanol and 3-[1-(3-bromophenyl)-3,3-difluorocyclobutyl]-4-methyl-1,2,4-triazole. ¹HNMR (400 MHz, CDCl₃) δ 8.18 (s, 1H), 7.64 (t, J=7.9 Hz, 1H), 7.48-7.38 (m, 1H), 7.28 (s, 1H), 7.21 (s, 1H), 6.57 (s, 1H), 6.54 (s, 1H), 4.57 (d, J=14.0 Hz, 1H), 4.38 (d, J=13.9 Hz, 1H), 4.13 (s, 1H), 3.77 (m, 2H), 3.42 (s, 5H), 1.74 (dd, J=8.4, 5.2 Hz, 1H), 1.21 (d, J=28.2 Hz, 2H), 1.10 (d, J=6.5 Hz, 3H), 0.95-0.72 (m, 4H), 0.48 (d, J=5.4 Hz, 2H). ESI MS [M+H]⁺ for C₂₉H₃₃F₂N₆O₂, calcd 535.6, found 535.1.

### Example 85: 4-cyclopropyl-6-{3-[3,3-difluoro-1-(4-methyl-4H-1,2,4-triazol-3-yl)cyclobutyl]phenyl}-2-[({[1-(hydroxymethyl)cyclopropyl]methyl}amino)methyl]-1H,6H,7H-pyrrolo[2,3-c]pyridin-7-one

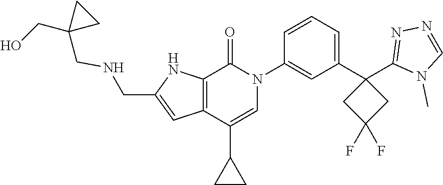 [SMILES: Cn1cnnc1C1(c2cccc(-n3cc(C4CC4)c4cc(CNCC5(CO)CC5)[nH]c4c3=O)c2)CC(F)(F)C1]

The title compound was prepared in a similar fashion to that described for Example 79 from 1-(aminomethyl)cyclopropanemethanol and 3-[1-(3-bromophenyl)-3,3-difluorocyclobutyl]-4-methyl-1,2,4-triazole. ¹H NMR (400 MHz, CDCl₃) δ 8.12 (s, 1H), 7.72-7.51 (m, 1H), 7.42-7.35 (m, 1H), 7.27 (d, J=1.3 Hz, 2H), 6.62 (d, J=1.5 Hz, 2H), 4.44 (s, 2H), 3.76 (q, J=13.1 Hz, 2H), 3.46 (s, 2H), 3.38 (s, 5H), 3.09 (s, 2H), 1.85-1.72 (m, 1H), 0.93-0.80 (m, 2H), 0.67 (s, 2H), 0.60-0.49 (m, 4H). ESI MS [M+H]⁻ for C₂₉H₃₃F₂N₆O₂, calcd 547.6, found 547.1.

### Example 86: 4-cyclopropyl-6-{3-[3,3-difluoro-1-(4-methyl-4H-1,2,4-triazol-3-yl)cyclobutyl]phenyl}-2-({4-oxa-7-azaspiro[2.5]octan-7-yl}methyl)-1H,6H,7H-pyrrolo[2,3-c]pyridin-7-one

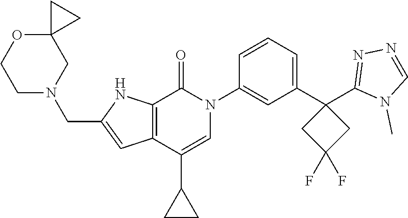 [SMILES: Cn1cnnc1C1(c2cccc(-n3cc(C4CC4)c4cc(CN5CCOC6(CC6)C5)[nH]c4c3=O)c2)CC(F)(F)C1]

The title compound was prepared in a similar fashion to that described for Example 79 from 4-oxa-7-azaspiro[2.5]octane hydrochloride and 3-[1-(3-bromophenyl)-3,3-difluorocyclobutyl]-4-methyl-1,2,4-triazole. ¹H NMR (400 MHz, CDCl₃) δ 8.10 (s, 1H), 7.50 (q, J=7.7 Hz, 2H), 7.42-7.35 (m, 1H), 7.28 (t, J=1.4 Hz, 1H), 6.73 (d, J=1.3 Hz, 1H), 6.64 (s, 1H), 4.26 (s, 2H), 3.99 (s, 2H), 3.76 (q, J=13.3 Hz, 2H), 3.38 (s, 5H), 2.95 (m, 4H), 1.93-1.81 (m, 1H), 1.80-1.64 (m, 2H), 0.98 (s, 2H), 0.92-0.83 (m, 2H), 0.70-0.57 (m, 2H). ESI MS [M+H]⁺ for C₃₀H₃₃F₂N₆O2, calcd 547.6, found 547.1.

### Example 87: (3R)-3-{[(4-cyclopropyl-6-{3-[3,3-difluoro-1-(4-methyl-4H-1,2,4-triazol-3-yl)cyclobutyl]phenyl}-7-oxo-1H,6H,7H-pyrrolo[2,3-c]pyridin-2-yl)methyl]amino}-1λ⁶-thiane-1,1-dione

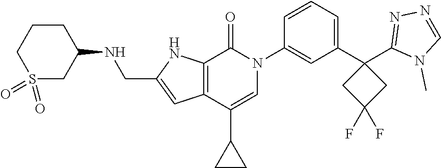

The title compound was prepared in a similar fashion to that described for Example 79 from (R)-3-aminotetrahydro-2H-thiopyran 1,1-dioxide and 3-[1-(3-bromophenyl)-3,3-difluorocyclobutyl]-4-methyl-1,2,4-triazole. ¹H NMR (400 MHz, Methanol-d4) δ 8.29 (s, 1H), 7.48 (t, J=7.8 Hz, 1H), 7.35-7.21 (m, 3H), 6.75 (d, J=1.2 Hz, 1H), 6.55 (s, 1H), 4.07 (s, 2H), 3.68-3.53 (m, 2H), 3.46-3.35 (m, 3H), 3.33 (s, 3H), 3.05-2.88 (m, 5H), 2.11 (d, J=16.1 Hz, 2H), 1.94-1.78 (m, 1H), 1.44 (d, J=12.5 Hz, 1H), 0.89-0.74 (m, 2H), 0.67-0.47 (m, 2H). ESI MS [M+H]⁺ for C₂₉H₃₂F₂N₆O₃S, calcd 583.6, found 583.1.

### Example 88: 2-({5-azaspiro[2.4]heptan-5-yl}methyl)-4-cyclopropyl-6-{3-[3,3-difluoro-1-(4-methyl-4H-1,2,4-triazol-3-yl)cyclobutyl]phenyl}-1H,6H,7H-pyrrolo[2,3-c]pyridin-7-one

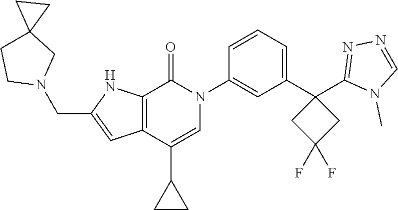 [SMILES: Cn1cnnc1C1(c2cccc(-n3cc(C4CC4)c4cc(CN5CCC6(CC6)C5)[nH]c4c3=O)c2)CC(F)(F)C1]

The title compound was prepared in a similar fashion to that described for Example 79 from 3-[1-(3-bromophenyl)-3,3-difluorocyclobutyl]-4-methyl-1,2,4-triazole. ¹H NMR (400 MHz, CDCl₃) δ 10.40 (s, 1H), 8.06 (s, 1H), 7.52-7.44 (m, 1H), 7.35-7.28 (m, 3H), 6.72 (d, J=1.3 Hz, 1H), 6.45 (s, 1H), 3.98 (s, 2H), 3.76 (q, J=13.3, 12.9 Hz, 2H), 3.37 (s, 4H), 2.98 (d, J=7.3 Hz, 2H), 2.72 (s, 2H), 1.98-1.83 (m, 3H), 0.91-0.80 (m, 2H), 0.63 (d, J=8.5 Hz, 6H). ESI MS [M+H]⁺ for C₃₀H₃₂F₂N₆O, calcd 531.6, found 531.1.

### Example 89: 4-cyclopropyl-6-{3-[3,3-difluoro-1-(4-methyl-4H-1,2,4-triazol-3-yl)cyclobutyl]phenyl}-2-({[1-(fluoromethyl)cyclobutyl]amino}methyl)-1H,6H,7H-pyrrolo[2,3-c]pyridin-7-one

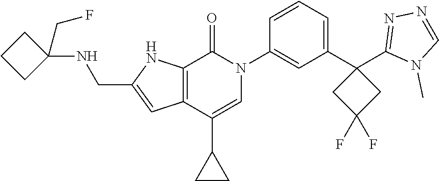 [SMILES: Cn1cnnc1C1(c2cccc(-n3cc(C4CC4)c4cc(CNC5(CF)CCC5)[nH]c4c3=O)c2)CC(F)(F)C1]

The title compound was prepared in a similar fashion to that described for Example 79 from 1-(fluoromethyl)cyclobutan-1-amine hydrochloride and 3-[1-(3-bromophenyl)-3,3-difluorocyclobutyl]-4-methyl-1,2,4-triazole. ¹H NMR (400 MHz, CDCl₃) δ 8.06 (s, 1H), 7.51 (t, J=8.0 Hz, 1H), 7.39-7.29 (m, 3H), 6.71-6.66 (m, 1H), 6.35 (s, 1H), 4.61 (s, 1H), 4.49 (s, 1H), 3.99 (s, 2H), 3.76 (q, J=12.8 Hz, 2H), 3.36 (s, 5H), 2.16 (s, 1H), 2.04-1.71 (m, 7H), 0.96-0.80 (m, 2H), 0.66-0.53 (m, 2H). ESI MS [M+H]⁺ for C₂₉H₃₂F₃N₆O, calcd 537.6, found 537.1.

### Example 90: 2-{[(4-chloro-2-fluorophenyl)amino]methyl}-4-cyclopropyl-6-{3-[3,3-difluoro-1-(4-methyl-4H-1,2,4-triazol-3-yl)cyclobutyl]phenyl}-1H,6H,7H-pyrrolo[2,3-c]pyridin-7-one

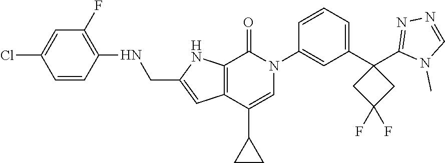 [SMILES: Cn1cnnc1C1(c2cccc(-n3cc(C4CC4)c4cc(CNc5ccc(Cl)cc5F)[nH]c4c3=O)c2)CC(F)(F)C1]

The title compound was prepared in a similar fashion to that described for Example 79 from 4-chloro-2-fluoroaniline and 3-[1-(3-bromophenyl)-3,3-difluorocyclobutyl]-4-methyl-1,2,4-triazole. ¹H NMR (400 MHz, CDCl₃) δ 10.84 (s, 1H), 8.02 (s, 1H), 7.54-7.40 (m, 1H), 7.37-7.31 (m, 1H), 7.32-7.27 (m, 2H), 6.94 (dd, J=11.2, 2.3 Hz, 1H), 6.92-6.86 (m, 1H), 6.71 (d, J=1.2 Hz, 1H), 6.56 (t, J=8.9 Hz, 1H), 6.48 (d, J=2.2 Hz, 1H), 4.68 (s, 1H), 4.41 (s, 2H), 3.83-3.66 (m, 3H), 3.47-3.30 (m, 2H), 3.27 (s, 3H), 1.89 (qd, J=8.9, 5.5 Hz, 1H), 1.06-0.83 (m, 2H), 0.70-0.57 (m, 2H). ESI MS [M+H]⁻ for C₃₀H₂₇ClF₃N₆O, calcd 580.1, found 580.1.

### Example 91: 4-cyclopropyl-6-{3-[3,3-difluoro-1-(4-methyl-4H-1,2,4-triazol-3-yl)cyclobutyl]phenyl}-2-{[(oxolan-3-yl)amino]methyl}-1H,6H,7H-pyrrolo[2,3-c]pyridin-7-one

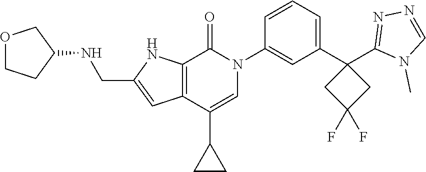

The title compound was prepared in a similar fashion to that described for Example 79 from (R)-3-aminotetrahydrofuran hydrochloride and 3-[1-(3-bromophenyl)-3,3-difluorocyclobutyl]-4-methyl-1,2,4-triazole. ¹H NMR (400 MHz, CDCl₃) δ 8.09 (s, 1H), 7.65-7.51 (m, 1H), 7.43 (d, J=8.0 Hz, 1H), 7.27 (s, 2H), 6.67 (s, 1H), 6.38 (s, 1H), 4.38 (s, 1H), 4.13 (d, J=5.6 Hz, 2H), 4.01 (ddd, J=22.9, 9.7, 5.9 Hz, 2H), 3.84-3.66 (m, 4H), 3.61 (dd, J=9.7, 4.0 Hz, 1H), 3.39 (s, 3H), 3.02-2.86 (m, 2H), 1.79 (m, 2H), 0.96-0.80 (m, 2H), 0.59 (q, J=5.3 Hz, 2H). ESI MS [M+H]⁺ for C₂₈H₃₁F₂N₆O₂, calcd 521.6, found 521.1.

### Example 92: 4-cyclopropyl-6-{3-[3,3-difluoro-1-(4-methyl-4H-1,2,4-triazol-3-yl)cyclobutyl]phenyl}-2-{[(oxolan-3-yl)amino]methyl}-1H,6H,7H-pyrrolo[2,3-c]pyridin-7-one

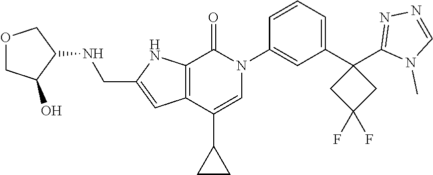

The title compound was prepared in a similar fashion to that described for Example 79 from (3R,4S)-4-aminotetrahydro-3-furanol hydrochloride and 3-[1-(3-bromophenyl)-3,3-difluorocyclobutyl]-4-methyl-1,2,4-triazole. ¹H NMR (400 MHz, CDCl₃) δ 12.68 (s, 1H), 8.16 (s, 1H), 7.74-7.45 (m, 2H), 7.21 (d, J=10.4 Hz, 2H), 6.59 (d, J=1.2 Hz, 1H), 6.32 (s, 1H), 4.68 (s, 1H), 4.27 (q, J=13.7 Hz, 2H), 4.02 (dt, J=34.3, 8.5 Hz, 2H), 3.93-3.69 (m, 3H), 3.65-3.49 (m, 2H), 3.42 (s, 3H), 3.03-2.82 (m, 2H), 1.71 (s, 2H), 0.83 (dd, J=8.3, 1.8 Hz, 2H), 0.53 (q, J=5.3 Hz, 2H). ESI MS [M+H]⁺ for C₂₈H₃₁F₂N₆O₃, calcd 537.6, found 537.1.

### Example 93: 4-cyclopropyl-6-{3-[3,3-difluoro-1-(4-methyl-4H-1,2,4-triazol-3-yl)cyclobutyl]phenyl}-2-{[(3R,4R)-4-fluoro-3-hydroxypiperidin-1-yl]methyl}-1H,6H,7H-pyrrolo[2,3-c]pyridin-7-one

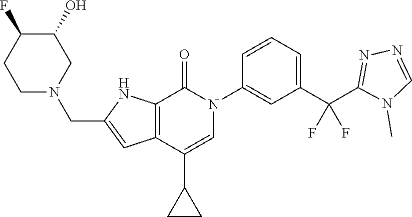 [SMILES: Cn1cnnc1C(F)(F)c1cccc(-n2cc(C3CC3)c3cc(CN4CC[C@@H](F)[C@H](O)C4)[nH]c3c2=O)c1]

The title compound was prepared in a similar fashion to that described for Example 79 from (3R,4R)-4-fluoropiperidin-3-ol hydrochloride and 3-[1-(3-bromophenyl)-3,3-difluorocyclobutyl]-4-methyl-1,2,4-triazole. ¹H NMR (400 MHz, CDCl₃) δ 11.66 (s, 1H), 8.25 (s, 1H), 7.63-7.50 (m, 1H), 7.42 (d, J=7.9 Hz, 1H), 7.28 (d, J=1.3 Hz, 1H), 7.17 (t, J=2.0 Hz, 1H), 6.75 (d, J=1.3 Hz, 1H), 6.67 (d, J=2.0 Hz, 1H), 4.38 (q, J=13.7 Hz, 2H), 4.16 (s, 1H), 3.73 (dt, J=26.8, 13.3 Hz, 2H), 3.38 (m, 4H), 3.20-2.99 (m, 2H), 2.62-2.41 (m, 2H), 2.08 (s, 1H), 1.85 (d, J=7.5 Hz, 2H), 1.25 (d, J=5.7 Hz, 1H), 0.96-0.78 (m, 2H), 0.62 (td, J=5.9, 4.3 Hz, 2H). ESI MS [M+H]⁺ for C₂₉H₃₂F₃N₆O₂, calcd 553.6, found 553.1.

### Example 94: 4-cyclopropyl-6-[3-[3,3-difluoro-1-(4-methyl-1,2,4-triazol-3-yl)cyclobutyl]phenyl]-2-[[(2R)-2-methylmorpholin-4-yl]methyl]-1H-pyrrolo[2,3-c]pyridin-7-one

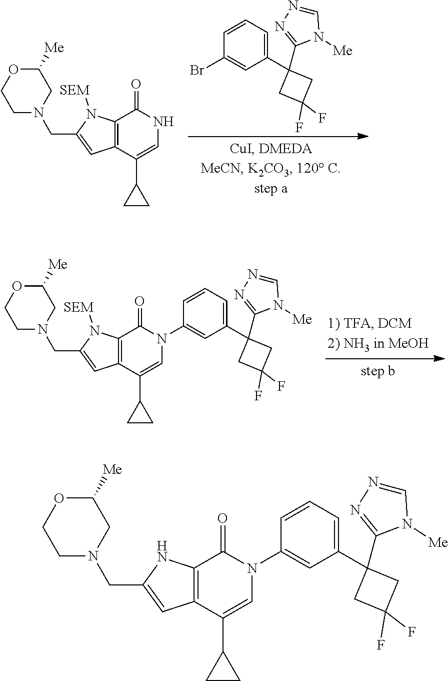 [SMILES: C[C@@H]1CN(Cc2cc3c(C4CC4)c[nH]c(=O)c3n2COCC[Si](C)(C)C)CCO1 ; Cn1cnnc1C1(c2cccc(Br)c2)CC(F)(F)C1 ; C[C@@H]1CN(Cc2cc3c(C4CC4)cn(-c4cccc(C5(c6nncn6C)CC(F)(F)C5)c4)c(=O)c3n2COCC[Si](C)(C)C)CCO1 ; C[C@@H]1CN(Cc2cc3c(C4CC4)cn(-c4cccc(C5(c6nncn6C)CC(F)(F)C5)c4)c(=O)c3[nH]2)CCO1]

The title compound was prepared in a similar fashion to that described for Example 79 from (R)-3-methylmorpholine and 3-[1-(3-bromophenyl)-3,3-difluorocyclobutyl]-4-methyl-1,2,4-triazole. ¹H NMR (400 MHz, CDCl₃) δ 9.81 (s, 1H), 8.06 (s, 1H), 7.53-7.47 (m, 1H), 7.36-7.28 (m, 3H), 6.73 (d, J=1.2 Hz, 1H), 6.42 (d, J=2.2 Hz, 1H), 3.88-3.80 (m, 1H), 3.80-3.73 (m, 2H), 3.73-3.58 (m, 4H), 3.42-3.27 (m, 5H), 2.70 (ddt, J=15.9, 11.2, 2.0 Hz, 2H), 2.23 (td, J=11.3, 3.3 Hz, 1H), 1.94-1.85 (m, 2H), 1.12 (d, J=6.3 Hz, 3H), 0.93-0.83 (m, 2H), 0.68-0.59 (m, 2H). ESI MS [M+H]⁺ for C₂₉H₃₂F₂N₆O₂, calcd 535.3, found 535.3.

### Example 95: trans-4-cyclopropyl-6-[3-[3-(difluoromethoxy)-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

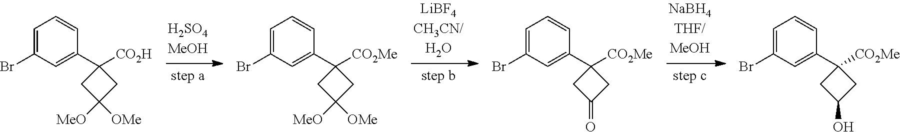 [SMILES: COC1(OC)CC(C(=O)O)(c2cccc(Br)c2)C1 ; COC(=O)C1(c2cccc(Br)c2)CC(OC)(OC)C1 ; COC(=O)C1(c2cccc(Br)c2)CC(=O)C1 ; COC(=O)[C@]1(c2cccc(Br)c2)C[C@@H](O)C1]

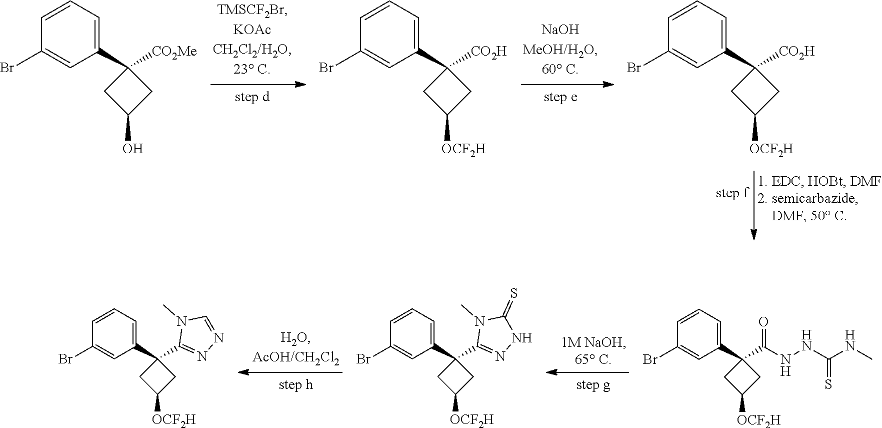

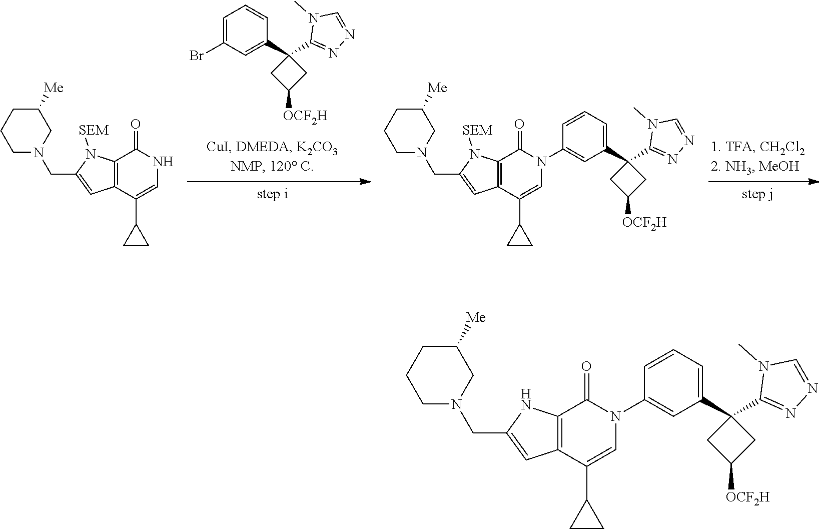

Step a: 1-(3-Bromophenyl)-3,3-dimethoxycyclobutane-1-carboxylic acid (6.2 g, 20.0 mmol) was dissolved in MeOH (100.0 mL), concentrated sulfuric acid (3.0 mL) was added, and the reaction mixture was refluxed for 1 h. The resulting solution was cooled to room temperature and concentrated to ˜10.0 mL under reduced pressure. The concentrate was diluted with water (100.0 mL) and ethyl acetate (100.0 mL), the organic phase was separated, and the aqueous phase was additionally extracted with EtOAc (2×30.0 mL). The combined organic extract was washed with aq. sat. sodium bicarbonate (100.0 mL) and brine (100.0 mL), dried over sodium sulfate and concentrated to dryness under reduced pressure to provide the corresponding ester.

Step b: Ester from step a and LiBF4 (2.0 g, 21.9 mmol) were dissolved in acetonitrile (63 mL) containing water (1.30 ml), and the solution was stirred at 60° C. for 1 h. The reaction was cooled to room temperature and diluted with water (150 mL) and ethyl acetate (100 mL). The organic phase was separated, additionally washed with water (100 mL) and brine (50 mL), dried over sodium sulfate and concentrated to dryness under reduced pressure to yield the corresponding 7-ketoester.

Step c: γ-Ketoester from step b (0.5 g, 1.8 mmol) was dissolved in a mixture of THF (9.0 mL) and MeOH (9.0 mL). The reaction mixture was cooled to −78° C. and a solution of NaBH₄ (134.0 mg, 3.5 mmol) in MeOH (2.0 mL) was added dropwise over 1 min. The resulting solution was maintained at −78° C. for 2 h. Once TLC analysis indicated the reaction completion it was diluted with ethyl acetate (30.0 mL) and 1M aq. HCl (15.0 mL). The organic phase was separated, and the aqueous phase was extracted with EtOAc (2×15.0 mL). The combined organic extract was washed with brine (30.0 mL), dried over sodium sulfate and concentrated to dryness under reduced pressure. The crude product was purified by column chromatography (SiO₂, hexanes/EtOAc gradient) to produce the desired trans-methyl 1-(3-bromophenyl)-3-hydroxycyclobutane-1-carboxylate (dr=20:1).

Step d: TMSCF₂Br (2.60 mL, 16.7 mmol) and KOAc (1.60 g, 16.7 mmol) were added to a solution of trans-methyl 1-(3-bromophenyl)-3-hydroxycyclobutane-1-carboxylate (0.95 g, 3.33 mmol) in CH₂Cl₂ (1.50 mL) and water (1.50 mL). The reaction was stirred for 2 h at 23° C. The mixture was next diluted with CH₂Cl₂ (7 mL), and the organic layer was washed with water (2×10 mL). The combined organic phase was dried over Na₂SO₄, concentrated and the dry residue (1.0 g) was used in the next step without further purification.

Step e: To a solution of methyl ester from step d (1.0 g) in MeOH (8.50 mL) was added aq. 2M NaOH solution (8.50 mL, 5.0 mmol) at ambient temperature. The resulting mixture was stirred for 2 h at 60° C. Once TLC analysis indicated complete consumption of the starting material, methanol from the reaction was evaporated, residue was acidified to pH=1 with 1M HCl and extracted with EtOAc (2×5 mL). The combined organic phases were washed with brine, dried over Na₂SO₄, and concentrated in vacuo to give the final product as colorless oil.

Step f: The product from step e (0.94 g, 2.93 mmol) was dissolved in DMF (15 mL), and EDC (0.84 g, 4.40 mmol) followed by HOBt (0.67 g, 4.40 mmol) were added. The reaction was stirred for 1 h at 23° C. The mixture was diluted with EtOAc (20 mL), and washed with water (3×20 mL). Organic layer was dried over Na₂SO₄ and evaporated to dryness. The dry residue was dissolved in DMF (15 mL), and 4-methyl-3-thiosemicarbazide (0.37, 3.50 mmol) was added. The reaction was stirred for 15 h at 50° C. After completion, reaction mixture was cooled to rt and dissolved with EtOAc (20 mL). The organic phase was washed with water (3×15 mL), dried over Na₂SO₄, concentrated and the dry residue was used in the next step without further purification.

Step g: The product from step f was treated with aq. 1M NaOH solution (15 mL) for 30 min at 65° C. The reaction mixture was cooled to rt, acidified to pH=1 with 1M HCl, and extracted with EtOAc (2×15 mL). The combined organic phase was washed with brine, dried over Na₂SO₄, concentrated and the crude residue was purified by column chromatography (SiO₂, EtOAc in hexanes, 0 to 100%) to give triazole thiol.

Step h: Acetic acid (1.0 mL) and hydrogen peroxide (0.50 mL) were added to a solution of the product from step g (0.70 g, 1.79 mmol) in CH₂Cl₂ (8.0 mL) at 0° C. The reaction was warmed up to room temperature and stirred for 16 h. The mixture was carefully basified to pH=14 with aq. 1 M NaOH and extracted with CH₂C₁₂ (2×8 mL). The combined organic phase was dried over Na₂SO₄, concentrated and the crude residue was used in the next step without further purification.

Step i: A solution of amide (0.10 g, 0.24 mmol), triazole from step h (0.13 g, 0.36 mmol) and K₂CO₃ (0.10 g, 0.72 mmol) in NMP (2.40 ml) was degassed with a stream of bubbling nitrogen for ten minutes. CuI (46 mg, 0.24 mmol) and DMEDA (48 μL, 0.48 mmol) were added, and the reaction was heated for 2 h at 100° C. The reaction mixture was cooled down, quenched with aq. NH₃Cl and extracted with EtOAc (2×7 mL). The combined organic phase was washed with water (2×5 mL), brine, dried over Na₂SO₄, and concentrated to dryness. The crude material was purified by flash chromatography (SiO₂, EtOAc in Hex 0 to 60%, then MeOH in CH₂Cl₂ 0 to 10%) to give pyrrolopyridone as white foam.

Step j: Pyrrolopyridone from step i (94 mg, 0.14 mmol) was dissolved in CH₂Cl₂ (0.75 mL) and TFA (0.75 mL) was added. The mixture was stirred for 1 h and then concentrated. To this crude material was added 7M NH₃ in methanol (3.0 mL) and the mixture was stirred for 30 min. The reaction mixture was concentrated, and the crude product was purified by prep-HPLC (20% to 90% MeCN/water, 0.1% TFA) to afford the title compound. ¹H NMR (400 MHz, CDCl₃) δ 9.72 (s, 1H), 8.05 (s, 1H), 7.46 (t, J=7.8 Hz, 1H), 7.35-7.17 (m, 3H), 6.72 (d, J=1.3 Hz, 1H), 6.38 (s, 1H), 6.16 (t, J=74.3 Hz, 1H), 4.80 (p, J=7.5 Hz, 1H), 3.60 (d, J=1.8 Hz, 2H), 3.46 (tt, J=9.8, 2.6 Hz, 2H), 3.32 (s, 3H), 2.91-2.70 (m, 4H), 1.98-1.80 (m, 2H), 1.74-1.47 (m, 5H), 0.94-0.78 (m, 6H), 0.69-0.58 (m, 2H). ESI MS [M+H]⁺ for C₃₁H₃₆F₂N₆O₂, calcd 563.3, found 563.3.

### Example 96: cis-4-cyclopropyl-6-[3-[3-(difluoromethoxy)-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

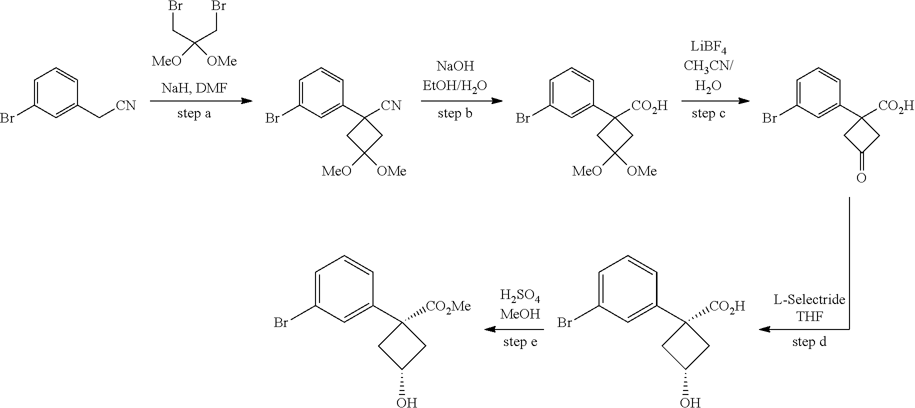

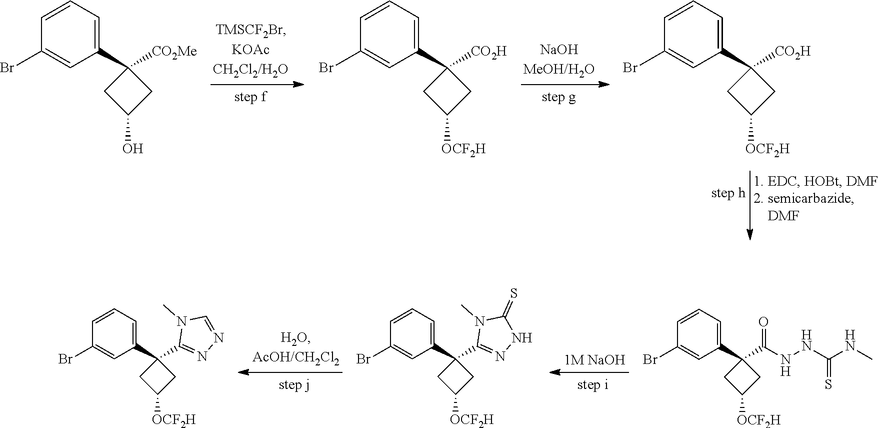

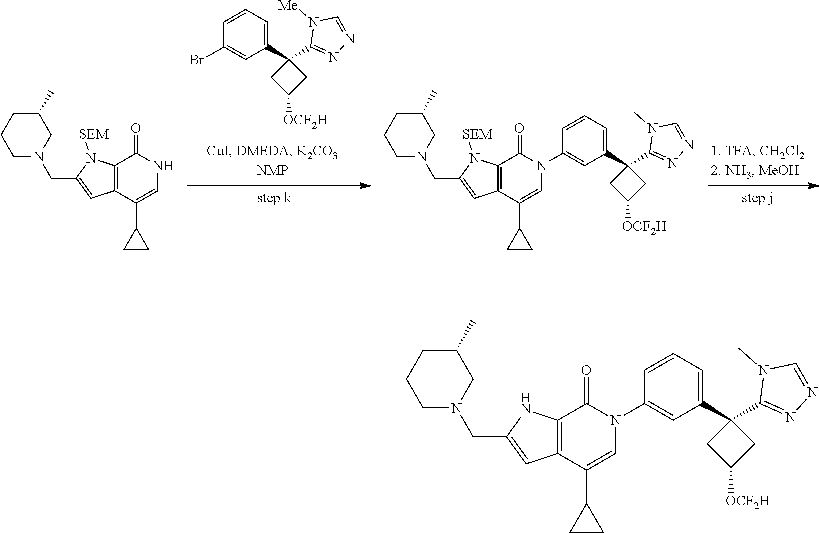

Step a: Sodium hydride (2.3 g, 57.0 mmol) was suspended in dry DMF (50.0 mL), and the resulting mixture was placed in 100 mL round bottom flask equipped with a magnetic stirring bar and reflux condenser with a drying tube. 2-(3-Bromophenyl)acetonitrile (5.0 g, 26.0 mmol) was added to the reaction mixture dropwise over 20 min. Once the addition was complete the cooling bath was removed, and the suspension was heated at 65° C. overnight. The resulting solution was carefully poured in aq. sat. NH₄Cl (100 mL). The biphasic mixture was diluted with ethyl acetate (100.0 mL) and water (100.0 mL). The organic phase was separated, and the aqueous phase was additionally extracted with ethyl acetate (2×50.0 mL). The combined organic extract was washed with water (2×70.0 mL) and brine (100 mL), dried over sodium sulfate and concentrated to dryness under reduced pressure. The crude product was purified by column chromatography (SiO₂, hexanes/EtOAc gradient) to produce the desired nitrile.

Step b: The nitrile from step a (3.5 g, 12.0 mmol) was refluxed overnight in a mixture of ethanol (20.0 mL) and water (20.0 mL) containing NaOH (4.8 g, 0.12 mol). The mixture was cooled to room temperature and EtOH was removed under reduced pressure. The resulting aqueous solution was diluted with water (50.0 mL), extracted with diethyl ether (3×50.0 mL) and acidified with 1M aq. hydrochloric acid to pH˜1. The product was extracted with ethyl acetate (3×50.0 mL). The combined organic extract was dried over sodium sulfate and concentrated to dryness under reduced pressure to provide the corresponding carboxylic acid.

Step c: A mixture of acid from step b (1.0 g, 3.2 mmol) and LiBF4 (0.33 g, 3.5 mmol) was dissolved in acetonitrile (10.0 mL) containing water (0.2 ml), and the solution was stirred at 60° C. for 1 h. The reaction was cooled to room temperature and diluted with water (30.0 mL) and ethyl acetate (20.0 mL). The organic phase was separated, additionally washed with water (30.0 mL) and brine (15.0 mL), dried over sodium sulfate and concentrated to dryness under reduced pressure to yield the corresponding γ-ketoacid.

Step d: A solution of γ-ketoacid from step c (0.5 g, 1.9 mmol) in THF (3 mL) was added to a solution of L-selectride (4.2 ml, 4.1 mmol, 1M solution) in THE preheated to 50° C. The reaction mixture was maintained at 50° C. for 2 h before it was cooled to room temperature. The reaction mixture was diluted with EtOAc (30.0 mL) and washed with 1M aq. hydrochloric acid (15.0 mL) and brine (15.0 mL). The organic phase was dried over sodium sulfate and concentrated to dryness to produce the desired γ-hydroxyacid as cis-isomer (dr=20:1).

Step e: γ-Hydroxyacid from step d (1.0 g, 3.7 mmol) was dissolved in MeOH (18.0 mL), concentrated sulfuric acid (0.1 mL) was added, and the reaction mixture was refluxed for 1 h. The resulting solution was cooled to room temperature and concentrated to ˜3.0 mL under reduced pressure. The concentrate was diluted with water (25.0 mL) and ethyl acetate (25.0 mL), the organic phase was separated, and the aqueous phase was additionally extracted with EtOAc (2×10.0 mL). The combined organic extract was washed with aq. sat. sodium bicarbonate (10.0 mL) and brine (10.0 mL), dried over sodium sulfate and concentrated to dryness under reduced pressure to provide cis-methyl 1-(3-bromophenyl)-3-hydroxycyclobutane-1-carboxylate.

Step f: TMSCF₂Br (2.60 mL, 16.7 mmol) and KOAc (1.60 g, 16.7 mmol) were added to the solution of trans-methyl 1-(3-bromophenyl)-3-hydroxycyclobutane-1-carboxylate (0.95 g, 3.33 mmol) in CH₂Cl₂ (1.50 mL) and water (1.50 mL). The reaction was stirred for 2 h at 23° C. The mixture was diluted with CH₂Cl₂ (7 mL) and the organic layer was washed with water (2×10 mL). The combined organic phase was dried over Na₂SO₄, concentrated and the dry residue (1.0 g) was used in the next step without further purification.

Step g: To a solution of methyl ester from step f (1.0 g) in MeOH (8.50 mL) was added aq. 2M NaOH solution (8.50 mL, 5.0 mmol) at ambient temperature. The resulting mixture was stirred for 2 h at 60° C. Once TLC analysis indicated complete consumption of the starting material, methanol from the reaction was evaporated, residue was acidified to pH=1 with 1M HCl and extracted with EtOAc (2×5 mL). The combined organic phases were washed with brine, dried over Na₂SO₄, and concentrated in vacuo to give the final product acid as colorless oil.

Step h: Product from step g (0.94 g, 2.93 mmol) was dissolved in DMF (15 mL), and EDC (0.84 g, 4.40 mmol) followed by HOBt (0.67 g, 4.40 mmol) were added. The reaction was stirred for 1 h at 23° C. The mixture was diluted with EtOAc (20 mL) and washed with water (3×20 mL). The organic layer was dried over Na₂SO₄ and evaporated to dryness. The dry residue was dissolved in DMF (15 mL), and 4-methyl-3-thiosemicarbazide (0.37, 3.50 mmol) was added. The reaction was stirred for 15 h at 50° C. After completion, the reaction mixture was cooled to rt and diluted with EtOAc (20 mL) and water (20 mL). The organic phase was washed with water (3×15 mL), dried over Na₂SO₄, concentrated and the dry residue was used in the next step without further purification.

Step i: The product from step h was treated with aq. 1M NaOH solution (15 mL) for 30 min at 65° C. The reaction mixture was cooled to rt, acidified to pH=1 with 1M HCl, and extracted with EtOAc (2×15 mL). The combined organic phase was washed with brine, dried over Na₂SO₄, concentrated and the crude residue was purified by column chromatography (SiO₂, EtOAc in hexanes, 0 to 100%) to give the desired triazole thiol.

Step j: Acetic acid (1.0 mL) and hydrogen peroxide (0.50 mL) were added to a solution of the product from step i (0.70 g, 1.79 mmol) in CH₂Cl₂ (8.0 mL) at 0° C. The reaction was warmed up to room temperature and let to stir for 16 h. The mixture was carefully basified to pH=14 with aq. 1 M NaOH and extracted with CH₂Cl₂ (2×8 mL). The combined organic phase was dried over Na₂SO₄, concentrated and the crude residue was used in the next step without further purification.

Step k: A solution of amide (0.10 g, 0.24 mmol), triazole from step j (0.13 g, 0.36 mmol) and K₂CO₃ (0.10 g, 0.72 mmol) in NMP (2.40 ml) was degassed with a stream of bubbling nitrogen for ten minutes. CuI (46 mg, 0.24 mmol) and DMEDA (48 μL, 0.48 mmol) were added, and the reaction was heated for 2 h at 100° C. The reaction mixture was cooled down, quenched with aq. NH₃Cl and extracted with EtOAc (2×7 mL). The combined organic phase was washed with water (2×5 mL), brine, dried over Na₂SO₄, and concentrated to dryness. The crude material was purified by flash chromatography (SiO₂, EtOAc in Hex 0 to 60%, then MeOH in CH₂Cl₂ 0 to 10%) to give pyrrolopyridone as a white foam.

Step 1: Pyrrolopyridone from step k (94 mg, 0.14 mmol) was dissolved in CH₂Cl₂ (0.75 mL) and TFA (0.75 mL) was added. The mixture was stirred for 1 h and then concentrated. To this crude material was added 7M NH₃ in methanol (3.0 mL) and the mixture was stirred for 30 min. The reaction mixture was concentrated, and the crude product was purified by prep-HPLC (20% to 90% MeCN/water, 0.1% TFA) to afford the title compound. ¹H NMR (400 MHz, CDCl₃) δ 9.82 (s, 1H), 7.97 (s, 1H), 7.52-7.42 (m, 2H), 7.31 (dddd, J=11.9, 7.7, 2.0, 1.0 Hz, 2H), 6.72 (d, J=1.3 Hz, 1H), 6.38 (s, 1H), 6.17 (t, J=73.8 Hz, 1H), 4.87 (p, J=7.7 Hz, 1H), 3.62-3.56 (m, 2H), 3.30 (s, 3H), 3.21 (tt, J=7.3, 1.8 Hz, 4H), 2.83-2.66 (m, 2H), 1.97-1.82 (m, 2H), 1.76-1.43 (m, 5H), 0.94-0.74 (m, 6H), 0.69-0.56 (m, 2H). ESI MS [M+H]⁺ for C₃₁H₃₆F₂N₆O₂, calcd 563.3, found 563.3.

### Example 97: 4-cyclopropyl-6-[3-[3-(difluoromethoxy)-1-(1-methylimidazol-2-yl)cyclobutyl]phenyl]-2-[[(3S)-3-methylpiperidin-1-yl]methyl]-1H-pyrrolo[2,3-c]pyridin-7-one

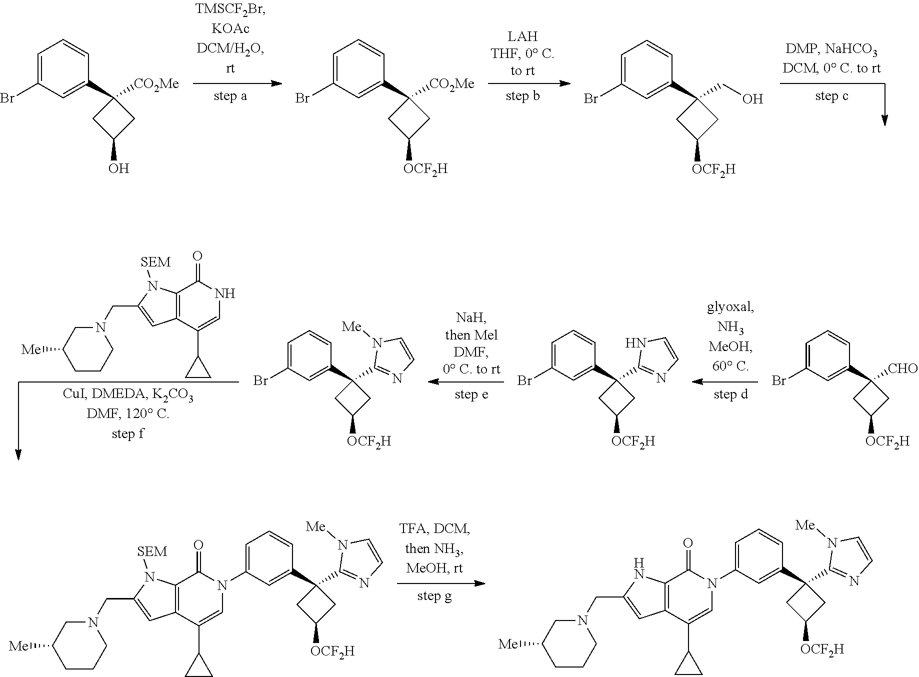

Step a: To a solution of trans-methyl 1-(3-bromophenyl)-3-hydroxycyclobutane-1-carboxylate (5.0 g, 17.5 mmol, 1.0 equiv.) in a 1:1 mixture of DCM/water (35 mL) was added TMSCF₂Br (13.7 mL, 88 mmol, 5.0 equiv.) and KOAc (8.63 g, 88 mmol, 4.0 equiv.). The reaction mixture was vigorously stirred at room temperature for 16 hours at which point it was partitioned between DCM (500 mL) and a ˜1:1 mixture of saturated aqueous NaCl/water (500 mL). The organics were dried over Na₂SO₄, filtered, and concentrated under vacuum. The crude residue was purified via silica gel flash column chromatography (0 to 100% EtOAc/hexanes) to afford the desired product and recovered starting material.

Step b: To a solution of the product from Step a (340 mg, 1.01 mmol, 1.0 equiv.) in THF (10 mL) at 0° C. was added LAH (2.0 M in THF, 1 mL, 2.0 mmol, 2.0 equiv.). The reaction was warmed to room temperature as the ice bath expired and allowed to stir for 2 hours at which point it was complete by TLC. The reaction mixture was cooled to 0° C. and 0.5 mL of 1.0 M NaOH was added dropwise followed by 2 mL of water. The reaction mixture was warmed to rt and stirred for 15 minutes. MgSO₄ was added and the slurry was stirred vigorously for an additional 15 minutes at rt. The solution was filtered and concentrated under vacuum and the crude residue was used directly in the subsequent step without further purification.

Step c: To a solution of the product from Step b (330 mg, 1.07 mmol, 1.0 equiv.) in DCM (10 mL) was added NaHCO₃ (180 mg, 2.14 mmol, 2.0 equiv.). The reaction mixture was cooled to 0° C., DMP (547 mg, 1.29 mmol, 1.2 equiv.) was added, and the reaction was stirred at 0° C. for 30 minutes, at which point it was warmed to room temperature and allowed to stir for an additional 1.5 hours. The reaction was quenched with saturated aqueous NaHCO₃ (20 mL) followed by saturated aqueous Na₂S₂O₃ (20 mL). The reaction mixture was partitioned between DCM (100 mL) and water (100 mL) and the organics were dried over Na₂SO₄, filtered, and concentrated under vacuum. The crude residue was used directly in the subsequent step without further purification.

Step d: To a solution of the product from Step c (245 mg, 0.80 mmol, 1.0 equiv.) in MeOH (4 mL) in a microwave vial was added glyoxal (260 uL, 5.6 mmol, 7.0 equiv.) and NH₃ (7.0 M in MeOH, 1.1 mL, 8.0 mmol, 10.0 equiv.). The vial was sealed and the reaction mixture was stirred at 60° C. for 2.5 hours. The reaction mixture was partitioned between EtOAc (50 mL) and water (50 mL) and the organics were dried over dried over Na₂SO₄, filtered, and concentrated under vacuum. The crude residue was purified via silica gel flash column chromatography (0 to 100% EtOAc/hexanes) to afford the desired product.

Step e: To a 40 mL vial was added NaH (9 mg, 0.40 mmol, 5.0 equiv.) followed by DMF (1 mL). The resulting suspension was cooled to 0° C. and the product from Step d (27 mg, 0.08 mmol, 1.0 equiv.) was added to the reaction mixture dropwise as a solution in DMF (1 mL). The reaction was stirred for 20 minutes at 0° C. at which point MeI (35 uL, 0.56 mmol, 7.0 equiv.) was added in a single portion. The reaction mixture was stirred for an additional 1 hour at rt at which point it was quenched with a ˜1:1 mixture of saturated aqueous NaCl/water (15 mL) and extracted with EtOAc (2×10 mL). The combined organics were dried over Na₂SO₄, filtered, and concentrated under vacuum. The crude residue was purified via silica gel flash column chromatography (0 to 100% EtOAc/hexanes) to afford the desired product.

Step f: To a solution of the product from Step e (20 mg, 0.06 mmol, 1.0 equiv.), 4-cyclopropyl-2-[[(3,S)-3-methylpiperidin-1-yl]methyl]-1-(2-trimethylsilylethoxymethyl)-6H-pyrrolo[2,3-c]pyridin-7-one (25 mg, 0.06 mmol, 1.0 equiv.), and K₂CO₃ (25 mg, 0.18 mmol, 3.0 equiv.) in DMF (1.2 mL) was added CuI (11 mg, 0.06 mmol, 1.0 equiv.) followed by DMEDA (13 uL, 0.12 mmol, 2.0 equiv.). The reaction mixture was heated to 120° C. and stirred for 2 hours at which point it was quenched with a ˜1:1 mixture of saturated aqueous NaCi/water (15 mL) and extracted with EtOAc (2×10 mL). The combined organics were dried over Na₂SO₄, filtered, and concentrated under vacuum. The crude residue was purified via silica gel flash column chromatography (0 to 20% MeOH/DCM) to afford the desired product.

Step g: A solution of the product from Step f (35 mg, 0.05 mmol, 1.0 equiv.) in a 1:1 mixture of TFA/DCM (1 mL) was stirred at room temperature for 1.5 hours. Toluene (5 mL) was added, and the reaction mixture was directly concentrated under vacuum. The crude residue was dissolved in 7N NH₃ in MeOH (1 mL) stirred for 20 minutes room temperature at which point it was directly concentrated under vacuum and the crude residue was purified via RP-HPLC (5 to 45% MeCN/H₂O) to afford the desired product. ¹H NMR (400 MHz, DMSO-d₆) δ 11.94 (s, 1H), 7.46 (t, J=7.8 Hz, 1H), 7.25 (d, J=8.1 Hz, 1H), 7.19-7.11 (m, 2H), 7.08 (d, J=1.2 Hz, 1H), 6.83 (d, J=1.2 Hz, 1H), 6.77 (d, J=1.1 Hz, 1H), 6.68 (t, J=75.7 Hz, 1H), 6.31 (d, J=2.1 Hz, 1H), 4.58-4.49 (m, 1H), 3.57 (s, 2H), 3.41-3.34 (m, 2H), 3.28 (s, 3H), 2.82-2.73 (m, 2H), 2.72-2.65 (m, 2H), 1.94-1.82 (m, 2H), 1.69-1.52 (m, 4H), 0.85-0.75 (m, 5H), 0.69-0.60 (m, 2H). ESI MS [M+H]⁺ for C₃₂H₃₇F₂N₅O₂, calcd 562.3, found 562.3.

### Example 98: trans-4-cyclopropyl-6-[3-[3-ethoxy-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

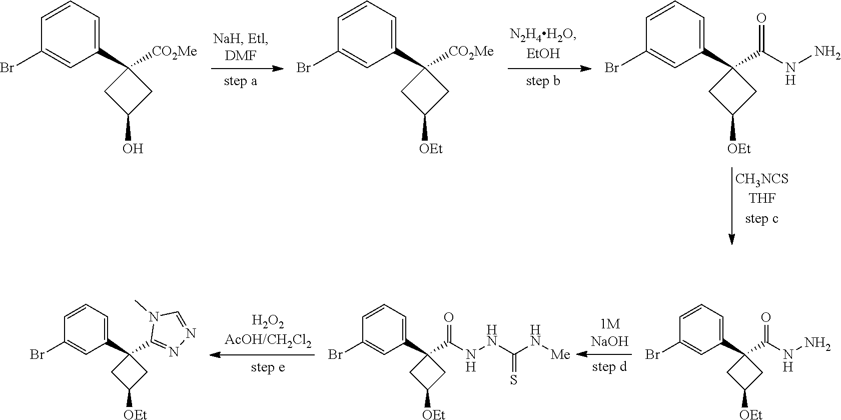 [SMILES: COC(=O)[C@]1(c2cccc(Br)c2)C[C@@H](O)C1 ; CCO[C@H]1C[C@@](C(=O)OC)(c2cccc(Br)c2)C1 ; CCO[C@H]1C[C@@](C(=O)NN)(c2cccc(Br)c2)C1 ; CCO[C@H]1C[C@](c2cccc(Br)c2)(c2nncn2C)C1 ; CCO[C@H]1C[C@@](C(=O)NNC(=S)NC)(c2cccc(Br)c2)C1 ; CCO[C@H]1C[C@@](C(=O)NN)(c2cccc(Br)c2)C1]

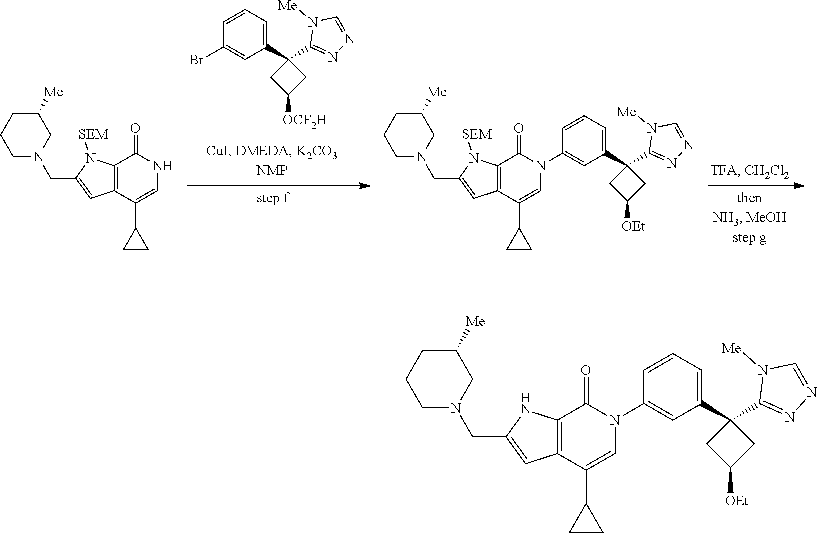

Step a: Ethyl iodide (0.34 mL, 4.25 mmol) was added to a suspension of trans-methyl 1-(3-bromophenyl)-3-hydroxycyclobutane-1-carboxylate (1.10 g, 3.86 mmol) and NaH (0.17 g, 4.25 mmol) in DMF (7.70 mL) at 0° C. The reaction was warmed up to room temperature, stirred for 2 h, then quenched with methanol, and diluted with EtOAc. The mixture was washed with water (3×5 mL), then brine, and the combined organic phase was dried over Na₂SO₄ and concentrated to dryness. The crude material was purified by flash chromatography (SiO₂, EtOAc in Hex 0 to 30%) to give mixture of methyl and ethyl ester as pale-yellow oil.

Step b: To a solution of product from step a (0.76 g) in EtOH (5.0 mL) was added hydrazine hydrate (1.20 mL, 24.0 mmol) at ambient temperature. The resulting mixture was stirred for 16 h at 80° C. The mixture was cooled down to room temperature and evaporated in vacuo. The resulting residue was diluted with water and extracted with EtOAc (3×5 mL). The combined organic phases were washed with brine, dried over Na₂SO₄, concentrated and the dry residue was used in the next step without further purification.

Step c: The product from step b was dissolved in THF (16 mL), and methylisothiocyanate (0.5 mL, 7.2 mmol) was added. The reaction was stirred for 2 h at 65° C. Upon completion, the reaction was concentrated, and the resulting residue was washed with MTBE and white solid (0.80 g) was filtered.

Step d: The product from step c (0.80 g) was treated with aq. 1M NaOH solution (12 mL) for 30 min at 65° C. The reaction mixture was cooled down, acidified to pH=1 with 1M HCl, and extracted with EtOAc (2×15 mL). The combined organic phase was washed with brine, dried over Na₂SO₄, concentrated and the crude residue was used in the next step.

Step e: Acetic acid (1.5 mL) and hydrogen peroxide (0.70 mL) were added to a solution of the product from step d in CH₂Cl₂ (12 mL) at 0° C. The reaction was warmed up to room temperature and stirred for 16 h. The mixture was carefully basified to pH=14 with aq. 1 M NaOH and extracted with CH₂Cl₂ (2×8 mL). The combined organic phase was dried over Na₂SO₄, concentrated and the crude residue was used in the next step without further purification.

Step f: A solution of amide (0.10 g, 0.24 mmol), triazole from step e (0.12 g, 0.36 mmol) and K₂CO₃ (0.10 g, 0.72 mmol) in NMP (2.40 ml) was degassed with a stream of bubbling nitrogen for ten minutes. CuI (46 mg, 0.24 mmol) and DMEDA (48 μL, 0.48 mmol) were added, and the reaction was heated for 2 h at 100° C. The reaction mixture was cooled down, quenched with aq. NH₃Cl, and extracted with EtOAc (2×7 mL). The combined organic phase was washed with water (2×5 mL), brine, dried over Na₂SO₄, and concentrated to dryness. The crude material was purified by reversed-phase C-18 modified flash chromatography (SiO₂, MeCN, 0.1% TFA/water, 0.1% TFA 0% to 100%) to give pyrrolopyridone that was used in the next step.

Step g: Pyrrolopyridone from step f (0.14 g) was dissolved in CH₂Cl₂ (1.20 mL) and TFA (1.20 mL) was added. The mixture was stirred for 1 h and then concentrated. To this crude material was added 7M NH₃ in methanol (3.0 mL) and the mixture was stirred for 30 min. The reaction mixture was concentrated, and the crude product was purified by prep-HPLC (20% to 90% MeCN/water, 0.1% TFA) to afford the title compound. ¹H NMR (400 MHz, CDCl₃) δ 10.20 (s, 1H), 8.04 (s, 1H), 7.44 (t, J=7.8 Hz, 1H), 7.30-7.19 (m, 2H), 6.71 (d, J=1.2 Hz, 1H), 6.41 (s, 1H), 4.14 (p, J=7.3 Hz, 1H), 3.73 (s, 2H), 3.44 (q, J=7.0 Hz, 2H), 3.38 (ddd, J=9.4, 6.9, 2.9 Hz, 2H), 3.32 (s, 3H), 2.90 (dd, J=21.5, 9.4 Hz, 2H), 2.64 (td, J=8.1, 2.8 Hz, 2H), 2.40 (s, 1H), 2.05 (dd, J=15.2, 10.1 Hz, 1H), 1.93-1.82 (m, 1H), 1.75 (d, J=7.7 Hz, 3H), 1.67 (d, J=7.7 Hz, 2H), 1.19 (t, J=7.0 Hz, 3H), 0.96-0.79 (m, 6H), 0.69-0.56 (m, 2H). ESI MS [M+H]⁺ for C₃₂H₄₀N₆O₂, calcd 541.3, found 541.3.

### Example 99: trans-4-cyclopropyl-6-[3-[3-hydroxy-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

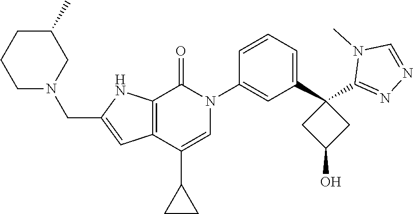 [SMILES: C[C@H]1CCCN(Cc2cc3c(C4CC4)cn(-c4cccc([C@]5(c6nncn6C)C[C@H](O)C5)c4)c(=O)c3[nH]2)C1]

The title compound was prepared in a similar fashion to Example 98 using TBSCl and imidazole in DMF in step a. ¹H NMR (400 MHz, CDCl₃) δ 10.20 (s, 1H), 8.04 (s, 1H), 7.44 (t, J=7.8 Hz, 1H), 7.30-7.19 (m, 2H), 6.71 (d, J=1.2 Hz, 1H), 6.41 (s, 1H), 4.14 (p, J=7.3 Hz, 1H), 3.73 (s, 2H), 3.44 (q, J=7.0 Hz, 2H), 3.32 (s, 3H), 2.90 (dd, J=21.5, 9.4 Hz, 2H), 2.64 (td, J=8.1, 2.8 Hz, 2H), 2.40 (s, 1H), 2.05 (dd, J=15.2, 10.1 Hz, 1H), 1.93-1.82 (m, 1H), 1.75 (d, J=7.7 Hz, 3H), 1.67 (d, J=7.7 Hz, 2H), 0.96-0.79 (m, 6H), 0.69-0.56 (m, 2H). ESI MS [M+H]⁺ for C₃₀H₃₆N₆O₂, calcd 513.3, found 513.3.

### Example 100: 2-({5-azaspiro[2.4]heptan-5-yl}methyl)-4-cyclopropyl-6-{3-[3,3-difluoro-1-(4-methyl-4H-1,2,4-triazol-3-yl)cyclobutyl]phenyl}-1H,6H,7H-pyrrolo[2,3-c]pyridin-7-one

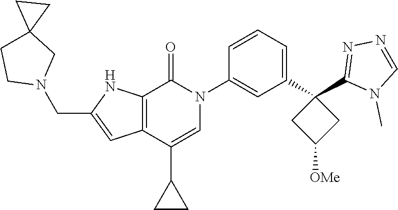 [SMILES: CO[C@H]1C[C@](c2cccc(-n3cc(C4CC4)c4cc(CN5CCC6(CC6)C5)[nH]c4c3=O)c2)(c2nncn2C)C1]

The title compound was prepared in a similar fashion to that described for Example 98 from 5-azaspiro[2.4]heptane and 3-[1-(3-bromophenyl)-3-methoxycyclobutyl]-4-methyl-1,2,4-triazole. ¹H NMR (400 MHz, CDCl₃) δ 10.80 (s, 1H), 8.05 (s, 1H), 7.45 (t, J=7.9 Hz, 1H), 7.25-7.16 (m, 3H), 6.71 (d, J=1.3 Hz, 1H), 6.49 (s, 1H), 4.25-3.96 (m, 3H), 3.39 (ddd, J=9.5, 6.9, 2.8 Hz, 1H), 3.32 (s, 3H), 3.27 (s, 3H), 3.11 (s, 2H), 2.83 (s, 2H), 2.62 (ddd, J=9.8, 7.6, 2.8 Hz, 2H), 1.95 (t, J=7.1 Hz, 2H), 1.91-1.81 (m, 1H), 0.94-0.79 (m, 2H), 0.72-0.54 (m, 7H). ESI MS [M+H]⁺ for C₃₁H37N₆O₂, calcd 525.7, found 525.1.

### Example 101: trans-4-cyclopropyl-6-[3-[3-methoxy-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

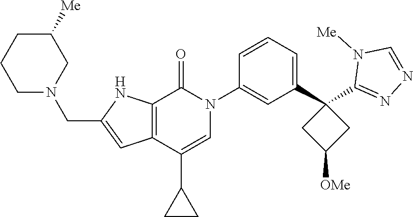 [SMILES: CO[C@H]1C[C@](c2cccc(-n3cc(C4CC4)c4cc(CN5CCC[C@H](C)C5)[nH]c4c3=O)c2)(c2nncn2C)C1]

The title compound was prepared in a similar fashion to Example 98 using methyl iodide in step a. ¹H NMR (400 MHz, CDCl₃) δ 9.60 (s, 1H), 8.03 (s, 1H), 7.44 (t, J=7.8 Hz, 1H), 7.33-7.18 (m, 2H), 6.71 (d, J=1.2 Hz, 1H), 6.37 (d, J=1.9 Hz, 1H), 4.07 (p, J=7.3 Hz, 1H), 3.58 (d, J=1.9 Hz, 2H), 3.46-3.36 (m, 2H), 3.32 (s, 3H), 3.27 (s, 3H), 2.84-2.72 (m, 2H), 2.62 (ddt, J=12.3, 7.4, 2.4 Hz, 2H), 1.99-1.82 (m, 3H), 1.74-1.47 (m, 5H), 0.96-0.77 (m, 6H), 0.69-0.60 (m, 2H). ESI MS [M+H]⁺ for C₃₁H₃₈N₆O₂, calcd 527.3, found 527.3.

### Example 102: cis-4-cyclopropyl-6-[3-[3-methoxy-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

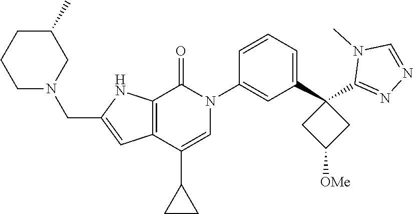 [SMILES: CO[C@H]1C[C@@](c2cccc(-n3cc(C4CC4)c4cc(CN5CCC[C@H](C)C5)[nH]c4c3=O)c2)(c2nncn2C)C1]

The title compound was prepared in a similar fashion to Example 98 using cis-methyl 1-(3-bromophenyl)-3-hydroxycyclobutane-1-carboxylate and methyl iodide in step a. ¹H NMR (400 MHz, CDCl₃) δ 9.66 (s, 1H), 7.96 (s, 1H), 7.51-7.41 (m, 2H), 7.37-7.24 (m, 2H), 6.72 (d, J=1.3 Hz, 1H), 6.40-6.33 (m, 1H), 4.14 (p, J=7.4 Hz, 1H), 3.59 (d, J=2.0 Hz, 2H), 3.31 (s, 3H), 3.25 (s, 3H), 3.17-3.05 (m, 2H), 3.00 (ddd, J=10.1, 6.4, 2.4 Hz, 2H), 2.85-2.71 (m, 2H), 2.07-1.81 (m, 3H), 1.75-1.46 (m, 5H), 0.96-0.77 (m, 6H), 0.69-0.59 (m, 2H).ESI MS [M+H]⁻ for C₃₁H₃₈N₆O₂, calcd 527.3, found 527.3.

### Example 103: 4-cyclopropyl-6-[3-[3-methoxy-1-(4-methyl-1,2,4-triazol-3-yl)cyclobutyl]phenyl]-2-[[(2R)-2-methylmorpholin-4-yl]methyl]-1H-pyrrolo[2,3-c]pyridin-7-one

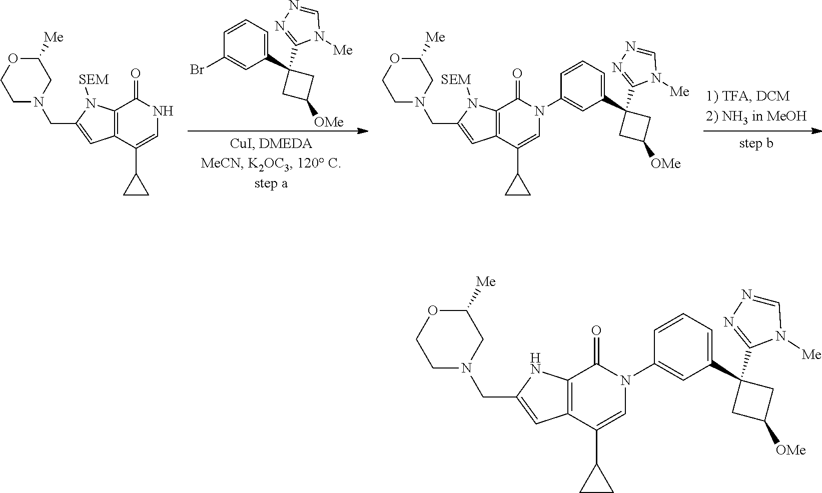 [SMILES: C[C@@H]1CN(Cc2cc3c(C4CC4)c[nH]c(=O)c3n2COCC[Si](C)(C)C)CCO1 ; CO[C@H]1C[C@](c2cccc(Br)c2)(c2nncn2C)C1 ; CO[C@H]1C[C@](c2cccc(-n3cc(C4CC4)c4cc(CN5CCO[C@H](C)C5)n(COCC[Si](C)(C)C)c4c3=O)c2)(c2nncn2C)C1 ; CO[C@H]1C[C@](c2cccc(-n3cc(C4CC4)c4cc(CN5CCO[C@H](C)C5)[nH]c4c3=O)c2)(c2nncn2C)C1]

Step a: To a solution of 4-cyclopropyl-2-[[(2R)-2-methylmorpholin-4-yl]methyl]-1-(2-trimethylsilylethoxymethyl)-6H-pyrrolo[2,3-c]pyridin-7-one (41.7 mg, 0.1 mmol), 3-[1-(3-bromophenyl)-3,3-difluorocyclobutyl]-4-methyl-4H-1,2,4-triazole (38.5 mg, 0.12 mmol), and DMEDA (13 μL, 0.12 mmol) in 1 mL MeCN, K₂CO₃ (41.4 mg, 0.3 mmol) was added in one portion. Nitrogen was bubbled into the solution for 10 mins followed by adding CuI (19 mg, 0.1 mmol). The reaction was stirred in sealed vial at 100° C. for 2 hours. After cooling to room temperature, saturated NH₄Cl was added, and the solution was extracted by DCM. The combined organic phase was dried over Na₂SO₄, concentrated and the crude residue was purified by column chromatography (SiO₂, 0-10% MeOH/DCM) to give the desired product. And product was used for the next step directly.

Step b: To a solution of the product from step a (60 mg, 0.09 mmol) in 2 mL DCM was added 2 mL TFA dropwise, and the reaction was stirred for 1 hour and concentrated under vacuum. The crude product was dissolved in 2 mL NH₃ in MeOH and stir for another 1 hour at room temperature before the crude product was concentrated and the crude residue was purified by prep-HPLC. 1H NMR (400 MHz, CDCl₃) δ 9.70 (s, 1H), 8.05 (s, 1H), 7.49-7.40 (m, 1H), 7.25-7.20 (m, 3H), 6.72 (d, J=1.3 Hz, 1H), 6.41 (d, J=2.0 Hz, 1H), 4.07 (p, J=7.2 Hz, 1H), 3.85 (ddd, J=11.4, 3.3, 1.4 Hz, 1H), 3.70-3.55 (m, 4H), 3.38 (ddd, J=11.9, 6.1, 2.6 Hz, 2H), 3.32 (s, 3H), 3.27 (s, 3H), 2.75-2.67 (m, 2H), 2.67-2.57 (m, 2H), 2.22 (td, J=11.3, 3.3 Hz, 1H), 1.93-1.83 (m, 2H), 1.12 (d, J=6.3 Hz, 3H), 0.92-0.84 (m, 2H), 0.70-0.59 (m, 2H). ESI MS [M+H]⁺ for C₃₀H₃₆N₆O₃, calcd 529.3, found 529.3.

### Example 104: 6-{3-[3-methyl-1-(4-methyl-4H-1,2,4-triazol-3-yl)cyclobutyl]phenyl}-2-{[(3S)-3-methylpiperidin-1-yl]methyl}-4-(trifluoromethyl)-1H,6H,7H-pyrrolo[2,3-c]pyridin-7-one

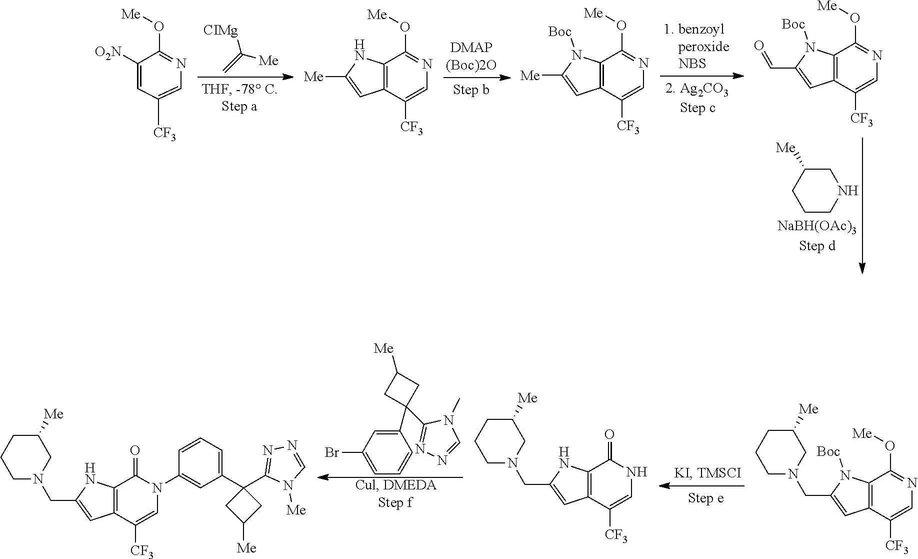

Step a: To a solution of 2-methoxy-3-nitro-5-(trifluoromethyl)pyridine (2.5 g, 11.25 mmol, 1.0 equiv.) in THF (25 mL, 0.45 M) was added isopropenylmagnesium bromide solution (66 ml, 33.76 mmol, 3.0 equiv.) at −78° C. The resulting solution was allowed to warm to rt and stirred for 3 h. The reaction was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexane, 0 to 15%) to give 7-methoxy-2-methyl-4-(trifluoromethyl)-1H-pyrrolo[2,3-c]pyridine.

Step b: To a solution of the product of step a (1.0 g, 4.3442 mmol, 1.0 equiv.) in THF (20 mL, 0.2 M) was added DMAP (53 mg, 0.4344 mmol, 0.1 equiv.), and di-tert-butyl dicarbonate (1050 mg, 4.7786 mmol, 1.1 equiv.) at 0° C. The resulting solution was stirred for 2 h at 0° C. The reaction was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexane, 0 to 15%) to give tert-butyl 7-methoxy-2-methyl-4-(trifluoromethyl)pyrrolo[2,3-c]pyridine-1-carboxylate.

Step c: To a solution of 2-methoxy-3-nitro-5-(trifluoromethyl)pyridine (940 mg, 2.8458 mmol, 1.0 equiv.) in CCl₄ (10 mL, 0.3 M) was added benzoyl peroxide (1010 mg, 5.6916 mmol, 2.0 equiv.), NBS (940 mg, 2.8458 mmol, 1.0 equiv.). The resulting solution was moved to a pre heated pad (80° C.) immediately and stirred for 2 h. The reaction was quenched with H₂O, the organic phase was separated, and the aqueous layer was extracted with EtOAc. The combined organic phase was dried over Na₂SO₄, concentrated. The crude residue was dissolved in acetone and AgCO₃ (1010 mg, 5.6916 mmol, 2.0 equiv.) was added. The resulting mixture was stirred for 12 h. The reaction was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexane, 0 to 15%) to give tert-butyl 2-formyl-7-methoxy-4-(trifluoromethyl)pyrrolo[2,3-c]pyridine-1-carboxylate.

Step d: To the product of step c (40 mg, 0.1638 mmol, 1.0 equiv.) in DCM (3 mL, 0.05 M) was added (S)-3-methylpiperidine hydrochloride (45 mg, 0.3276 mmol, 2.0 equiv.) and DIPEA (0.05 mL, 0.3276 mmol, 2.0 equiv.) and the mixture was stirred at rt for 10 mins. NaBH(OAc)₃ (73 mg, 0.3276 mmol, 2.0 equiv.) was added and the mixture was stirred at rt for 12 h. The reaction was quenched with NaHCO₃ sat., the organic phase was separated, and the aqueous layer was extracted with EtOAc. The combined organic phase was dried over Na₂SO₄, concentrated and the crude residue was purified by column chromatography (SiO₂, MeOH in DCM, 0 to 15%) to give tert-butyl 7-methoxy-2-[[(3S)-3-methylpiperidin-1-yl]methyl]-4-(trifluoromethyl)pyrrolo[2,3-c]pyridine-1-carboxylate.

Step e: To a solution of the product from step d (44 mg, 0.1283 mmol, 1.0 equiv.) and KI (34 mg, 0.2052 mmol, 1.6 equiv.) in CH₃CN (3 mL, 0.04 M) was added TMSCl (22 mg, 0.2052 mmol, 1.6 equiv.) dropwise at rt. H₂O (0.01 ml) was added into the solution. The resulting mixture was stirred at rt for 12 h. The mixture was then quenched with H₂O. The organic phase was separated, and the aqueous phase was extracted with EtOAc, the combined organic phase was then washed with brine, dried over Na₂SO₄, concentrated, and the crude residue was purified by column chromatography (SiO₂, MeOH in DCM, 0 to 20%) to give 2-[[(3S)-3-methylpiperidin-1-yl]methyl]-4-(trifluoromethyl)-1,6-dihydropyrrolo[2,3-c]pyridin-7-one.

Step f: To a solution of the product of step e (35 mg, 0.1117 mmol, 1.0 equiv.) and 3-[1-(3-bromophenyl)-3-methylcyclobutyl]-4-methyl-1,2,4-triazole (52 mg, 0.1676 mmol, 1.5 equiv.) in dioxane (3 mL, 0.03M) was added CuI (21 mg, 0.1117 mmol, 1.0 equiv.), DMEDA (40 mg, 0.4468 mmol, 4.0 equiv.) and K₂CO₃ (46 mg, 0.3351 mmol, 3.0 equiv.). The resulting solution was stirred at 110° C. for 2 h. The reaction was quenched with H₂O, the organic phase was separated, and the aqueous layer was extracted with EtOAc. The combined organic phase was dried over Na₂SO₄, concentrated and the crude residue was purified by column chromatography (MeOH in DCM, 0 to 20%) to give the crude mixture. The mixture was further was purified by prep-HPLC to furnish the title compound. ¹H NMR (400 MHz, CDCl₃) δ 10.43 (s, 1H), 8.01 (d, J=24.8 Hz, 1H), 7.53-7.46 (m, 2H), 7.41 (d, J=8.0 Hz, 2H), 7.29 (d, J=12.7 Hz, 1H), 6.40 (s, 1H), 3.74 (s, 2H), 3.29 (d, J=22.6 Hz, 3H), 2.89 (d, J=12.6 Hz, 4H), 2.77-2.57 (m, 2H), 2.09 (s, 1H), 1.84-1.58 (m, 4H), 1.14 (d, J=4.8 Hz, 3H), 0.87 (d, J=5.3 Hz, 4H). ESI MS [M+H]⁺ for C₂₉H₃₄F₃N₆O, calcd 539.6, found 539.1.

### Example 105: 4-cyclopropyl-2-{[(3S,5S)-3,5-difluoropiperidin-1-yl]methyl}-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

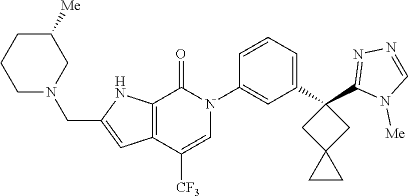

The title compound was prepared in a similar fashion to that described for Example 104. ¹H NMR (400 MHz, CDCl₃) δ 8.07 (s, 1H), 7.58-7.37 (m, 3H), 7.28 (m, 2H), 6.42 (s, 1H), 3.81 (s, 2H), 3.32 (m, 5H), 3.09-2.86 (m, 2H), 2.86-2.66 (m, 3H), 2.16 (s, 1H), 1.79 (d, J=14.9 Hz, 4H), 0.89 (d, J=5.8 Hz, 4H), 0.65-0.49 (m, 4H). ESI MS [M+H]⁺ for C₃₀H₃₄F₃N₆O, calcd 551.6, found 551.1.

### Example 106: 4-cyclopropyl-2-{[(3S,5S)-3,5-difluoropiperidin-1-yl]methyl}-6-{3-[(1r,3s)-3-methyl-1-(4-methyl-4H-1,2,4-triazol-3-yl)cyclobutyl]phenyl}-1H,6H,7H-pyrrolo[2,3-c]pyridin-7-one

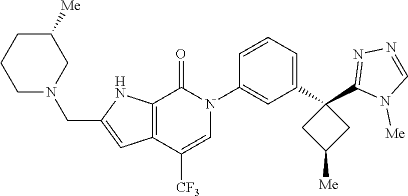 [SMILES: C[C@H]1CCCN(Cc2cc3c(C(F)(F)F)cn(-c4cccc([C@]5(c6nncn6C)C[C@@H](C)C5)c4)c(=O)c3[nH]2)C1]

The title compound was prepared in a similar fashion to that described for Example 104. ¹H NMR (400 MHz, CDCl₃) δ 9.76 (s, 1H), 7.97 (s, 1H), 7.56-7.46 (m, 2H), 7.44-7.36 (m, 2H), 7.28 (dd, J=2.1, 1.2 Hz, 1H), 6.42-6.31 (m, 1H), 3.70-3.54 (m, 2H), 3.27 (s, 3H), 2.95-2.83 (m, 2H), 2.81-2.61 (m, 4H), 1.96 (td, J=11.3, 3.0 Hz, 1H), 1.71-1.51 (m, 5H), 1.18-1.00 (m, 3H), 1.03-0.79 (m, 4H). ESI MS [M+H]⁻ for C₂₉H₃₄F₃N₆O, calcd 539.6, found 539.1.

### Example 107: 6-[3-[3-methyl-1-(4-methyl-1,2,4-triazol-3-yl)cyclobutyl]phenyl]-2-[[(3S)-3-methylpiperidin-1-yl]methyl]-4-methylsulfonyl-1H-pyrrolo[2,3-c]pyridin-7-one

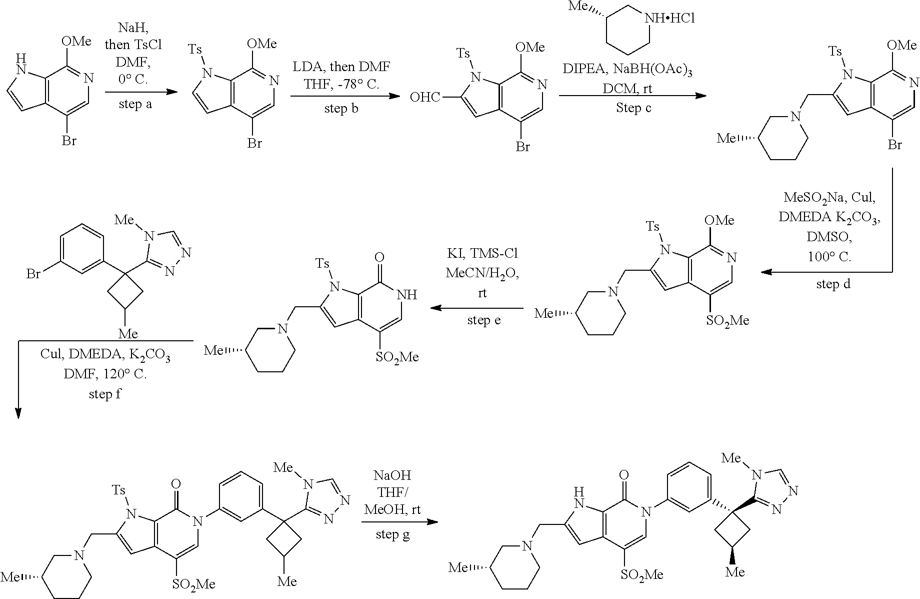

Step a: To a 40 mL vial was added NaH (170 mg, 4.23 mmol, 1.2 equiv.) followed by DMF (7 mL). The resulting suspension was cooled to 0° C. and 4-bromo-7-methoxy-1H-pyrrolo[2,3-c]pyridine (800 mg, 3.52 mmol, 1.0 equiv.) was added to the reaction mixture dropwise as a solution in DMF (3 mL). The reaction was stirred for 1 hour at 0° C. at which point p-TsCl (1.00 g, 5.28 mmol, 1.5 equiv.) was added in a single portion. The reaction mixture was stirred for an additional 45 minutes at 0° C. at which point it was poured into a ˜1:1 mixture of saturated aqueous NaCl/water (100 mL) and extracted with EtOAc (2×50 mL). The combined organics were dried over Na₂SO₄, filtered, and concentrated under vacuum. The crude residue was purified via silica gel flash column chromatography (0 to 50% EtOAc/hexanes) to afford the desired product.

Step b: To a solution of the product from Step a (256 mg, 0.67 mmol, 1.0 equiv.) in THF (4.5 mL) at −78° C. was added LDA (2.0 M in THF, 0.40 mL, 0.80 mmol, 1.2 equiv.). The reaction was stirred for 1.5 hours at −78° C. at which point DMF (80 uL, 1.00 mmol, 1.5 equiv.) was added as a solution in THF (1 mL). The reaction was stirred for an additional 30 minutes at −78° C., at which point it was quenched with water (5 mL) and warmed to room temperature. The mixture was partitioned between saturated aqueous NaCl (50 mL) and EtOAc (50 mL) and the organics were dried over Na₂SO₄, filtered, and concentrated under vacuum. The crude residue was purified via silica gel flash column chromatography (0 to 35% EtOAc/hexanes) to afford the desired product.

Step c: To a solution of the product from Step b (118 mg, 0.29 mmol, 1.0 equiv.) and (3S)-3-methylpiperidine hydrochloride (79 mg, 0.58 mmol, 2.0 equiv.) in DCM (3 mL) was added DIPEA (125 uL, 0.72 mmol, 2.5 equiv.). Sodium triacetoxyborohydride (160 mg, 0.72 mmol, 2.5 equiv.) was added in a single portion and the reaction was stirred at room temperature for 15 minutes at which point it was quenched with water (15 mL) and extracted with DCM (2×10 mL). The combined organics were dried over Na₂SO₄, filtered, and concentrated under vacuum. The crude residue was purified via silica gel flash column chromatography (0 to 50% EtOAc/hexanes) to afford the desired product.

Step d: To a solution of the product from Step c (111 mg, 0.23 mmol, 1.0 equiv.), sodium methanesulfinate (81 mg, 0.68 mmol, 3.0 equiv.), and K₂CO₃ (94 mg, 0.68 mmol, 3.0 equiv.) in DMSO (2.3 mL) was added CuI (44 mg, 0.23 mmol, 1.0 equiv.) followed by DMEDA (50 uL, 0.46 mmol, 2.0 equiv.). The reaction mixture was heated to 100° C. and stirred for 1 hour at which point it was quenched with a ˜1:1 mixture of saturated aqueous NaCl/water (15 mL) and extracted with EtOAc (2×10 mL). The combined organics were dried over Na₂SO₄, filtered, and concentrated under vacuum. The crude residue was purified via silica gel flash column chromatography (0 to 60% EtOAc/hexanes) to afford the desired product.

Step e: To a solution of the product from Step d (45 mg, 0.09 mmol, 1.0 equiv.) and KI (24 mg, 0.15 mmol, 1.6 equiv.) in MeCN (1 mL) was added water (5 uL, 0.28 mmol, 3.0 equiv.). TMS-Cl (20 uL, 0.15 mmol, 1.6 equiv.) was added and the reaction mixture was stirred at room temperature for 30 minutes. LCMS indicated partial conversion of the starting material. A second set of reagents was added to the reaction mixture, and it was stirred for an additional 15 minutes at room temperature at which point LCMS indicated full conversion. The reaction was quenched with a ˜1:1 mixture of saturated aqueous NaCl/water (15 mL) and extracted with EtOAc (2×10 mL). The combined organics were dried over Na₂SO₄, filtered, and concentrated under vacuum. The crude residue was used directly in the subsequent step without further purification.

Step f: To a solution of the product from Step e (44 mg, 0.09 mmol, 1.0 equiv.), 3-[1-(3-bromophenyl)-3-methylcyclobutyl]-4-methyl-1,2,4-triazole (34 mg, 0.11 mmol, 1.2 equiv.), and K₂CO₃ (39 mg, 0.28 mmol, 3.0 equiv.) in DMF (1 mL) was added CuI (17 mg, 0.09 mmol, 1.0 equiv.) followed by DMEDA (19 uL, 0.18 mmol, 2.0 equiv.). The reaction mixture was heated to 120° C. and stirred for 1.5 hours at which point it was quenched with a ˜1:1 mixture of saturated aqueous NaCl/water (15 mL) and extracted with EtOAc (2×10 mL). The combined organics were dried over Na₂SO₄, filtered, and concentrated under vacuum. The crude residue was purified via silica gel flash column chromatography (0 to 20% MeOH/DCM) to afford the desired product as a mixture of diastereomers.

Step g: To a solution of the product from Step f (27 mg, 0.04 mmol, 1.0 equiv.) in a 3:1 mixture of THF/MeOH (1.6 mL) was added NaOH (1.0 M in water, 0.2 mL, 0.2 mmol, 5.0 equiv.). The reaction mixture was stirred at room temperature for 2 hours at which point it was quenched with a ˜1:1 mixture of saturated aqueous NaCl/water (15 mL) and extracted with EtOAc (2×10 mL). The combined organics were dried over Na₂SO₄, filtered, and concentrated under vacuum. The crude residue was purified via RP-HPLC (10 to 45% MeCN/H₂O) to afford the desired product as a single diastereomer (first-eluting diastereomer off the HPLC). ¹H NMR (400 MHz, CDCl₃) δ 7.98 (s, 1H), 7.80 (s, 1H), 7.54-7.44 (m, 3H), 7.41 (t, J=2.1 Hz, 1H), 6.55 (s, 1H), 3.62 (d, J=2.1 Hz, 2H), 3.26 (s, 3H), 3.18 (s, 3H), 2.96-2.83 (m, 2H), 2.82-2.65 (m, 3H), 1.98 (t, J=11.6 Hz, 1H), 1.78-1.46 (m, 7H), 1.15 (d, J=5.6 Hz, 3H), 0.85 (d, J=5.8 Hz, 3H). ESI MS [M+H]⁺ for C₂₉H₃₆N₆O₃S, calcd 549.3, found 549.3.

### Example 108: 6-[3-[3-methyl-1-(4-methyl-1,2,4-triazol-3-yl)cyclobutyl]phenyl]-4-methylsulfonyl-1H-pyrrolo[2,3-c]pyridin-7-one

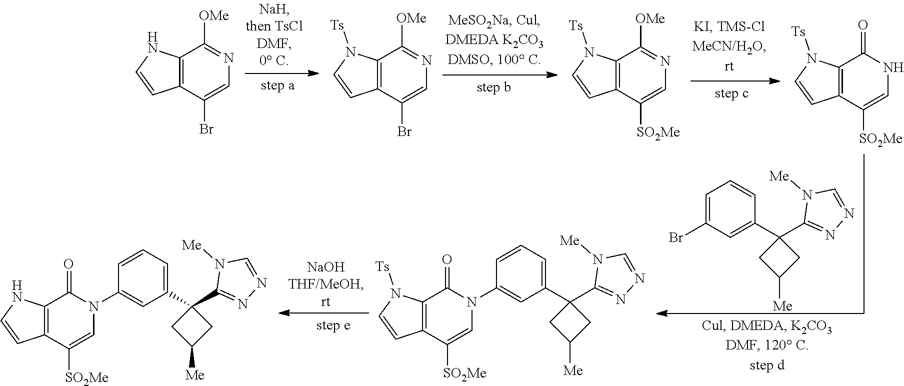

Step a: To a 40 mL vial was added NaH (75 mg, 1.85 mmol, 1.2 equiv.) followed by DMF (3 mL). The resulting suspension was cooled to 0° C. and 4-bromo-7-methoxy-1H-pyrrolo[2,3-c]pyridine (350 mg, 1.54 mmol, 1.0 equiv.) was added to the reaction mixture dropwise as a solution in DMF (1.5 mL). The reaction was stirred for 1 hour at 0° C. at which point p-TsCl (441 mg, 2.31 mmol, 1.5 equiv.) was added in a single portion. The reaction mixture was stirred for an additional 45 minutes at 0° C. at which point it was poured into a ˜1:1 mixture of saturated aqueous NaCl/water (50 mL) and extracted with EtOAc (2×30 mL). The combined organics were dried over Na₂SO₄, filtered, and concentrated under vacuum. The crude residue was purified via silica gel flash column chromatography (0 to 50% EtOAc/hexanes) to afford the desired product.

Step b: To a solution of the product from Step a (298 mg, 0.78 mmol, 1.0 equiv.), sodium methanesulfinate (140 mg, 1.17 mmol, 1.5 equiv.), and K₂CO₃ (323 mg, 2.34 mmol, 3.0 equiv.) in DMSO (8 mL) was added CuI (149 mg, 0.78 mmol, 1.0 equiv.) followed by DMEDA (170 uL, 1.56 mmol, 2.0 equiv.). The reaction mixture was heated to 100° C. and stirred for 1.5 hours at which point it was quenched with a ˜1:1 mixture of saturated aqueous NaCl/water (100 mL) and extracted with EtOAc (2×50 mL). The combined organics were dried over Na₂SO₄, filtered, and concentrated under vacuum. The crude residue was purified via silica gel flash column chromatography (0 to 100% EtOAc/hexanes) to afford the desired product.

Step c: To a solution of the product from Step b (38 mg, 0.10 mmol, 1.0 equiv.) and KI (27 mg, 0.16 mmol, 1.6 equiv.) in MeCN (1 mL) was added water (5 uL, 0.30 mmol, 3.0 equiv.). TMS-Cl (20 uL, 0.16 mmol, 1.6 equiv.) was added and the reaction mixture was stirred at room temperature for 1 hour. The reaction was quenched with a ˜1:1 mixture of saturated aqueous NaCl/water (15 mL) and extracted with a 3:1 mixture of CHCl₃/iPrOH (2×10 mL). The combined organics were dried over Na₂SO₄, filtered, and concentrated under vacuum. The crude residue was used directly in the subsequent step without further purification.

Step d: To a solution of the product from Step c (40 mg, 0.11 mmol, 1.0 equiv.), 3-[1-(3-bromophenyl)-3-methylcyclobutyl]-4-methyl-1,2,4-triazole (50 mg, 0.16 mmol, 1.5 equiv.), and K₂CO₃ (46 mg, 0.33 mmol, 3.0 equiv.) in DMF (1 mL) was added CuI (21 mg, 0.11 mmol, 1.0 equiv.) followed by DMEDA (24 uL, 0.22 mmol, 2.0 equiv.). The reaction mixture was heated to 120° C. and stirred for 3 hours at which point it was quenched with a ˜1:1 mixture of saturated aqueous NaCl/water (15 mL) and extracted with EtOAc (2×10 mL). The combined organics were dried over Na₂SO₄, filtered, and concentrated under vacuum. The crude residue was purified via silica gel flash column chromatography (0 to 20% MeOH/DCM) to afford the desired product as a mixture of diastereomers.

Step e: To a solution of the product from Step f (25 mg, 0.04 mmol, 1.0 equiv.) in a 3:1 mixture of THF/MeOH (1.6 mL) was added NaOH (1.0 M in water, 0.2 mL, 0.2 mmol, 5.0 equiv.). The reaction mixture was stirred at room temperature for 1 hour at which point it was quenched with a ˜1:1 mixture of saturated aqueous NaCl/water (15 mL) and extracted with EtOAc (2×10 mL). The combined organics were dried over Na₂SO₄, filtered, and concentrated under vacuum. The crude residue was purified via RP-HPLC (10 to 45% MeCN/H₂O) to afford the desired product as a single diastereomer (first-eluting diastereomer off the HPLC). ¹H NMR (400 MHz, CDCl₃) δ 9.96 (s, 1H), 7.99 (s, 1H), 7.84 (s, 1H), 7.57-7.46 (m, 2H), 7.44-7.38 (m, 2H), 7.28 (dt, J=7.7, 1.7 Hz, 1H), 6.79 (t, J=2.6 Hz, 1H), 3.26 (s, 3H), 3.20 (s, 3H), 2.95-2.82 (m, 2H), 2.76-2.64 (m, 3H), 1.15 (d, J=5.4 Hz, 3H). ESI MS [M+H]⁺ for C₂₂H₂₃N₅O₃S, calcd 438.2, found 438.2.

### Example 109: 7-cyclopropyl-5-[3-[3-methyl-1-(4-methyl-1,2,4-triazol-3-yl)cyclobutyl]phenyl]-2-[[(3S)-3-methylpiperidin-1-yl]methyl]-3H-imidazo[4,5-c]pyridin-4-one

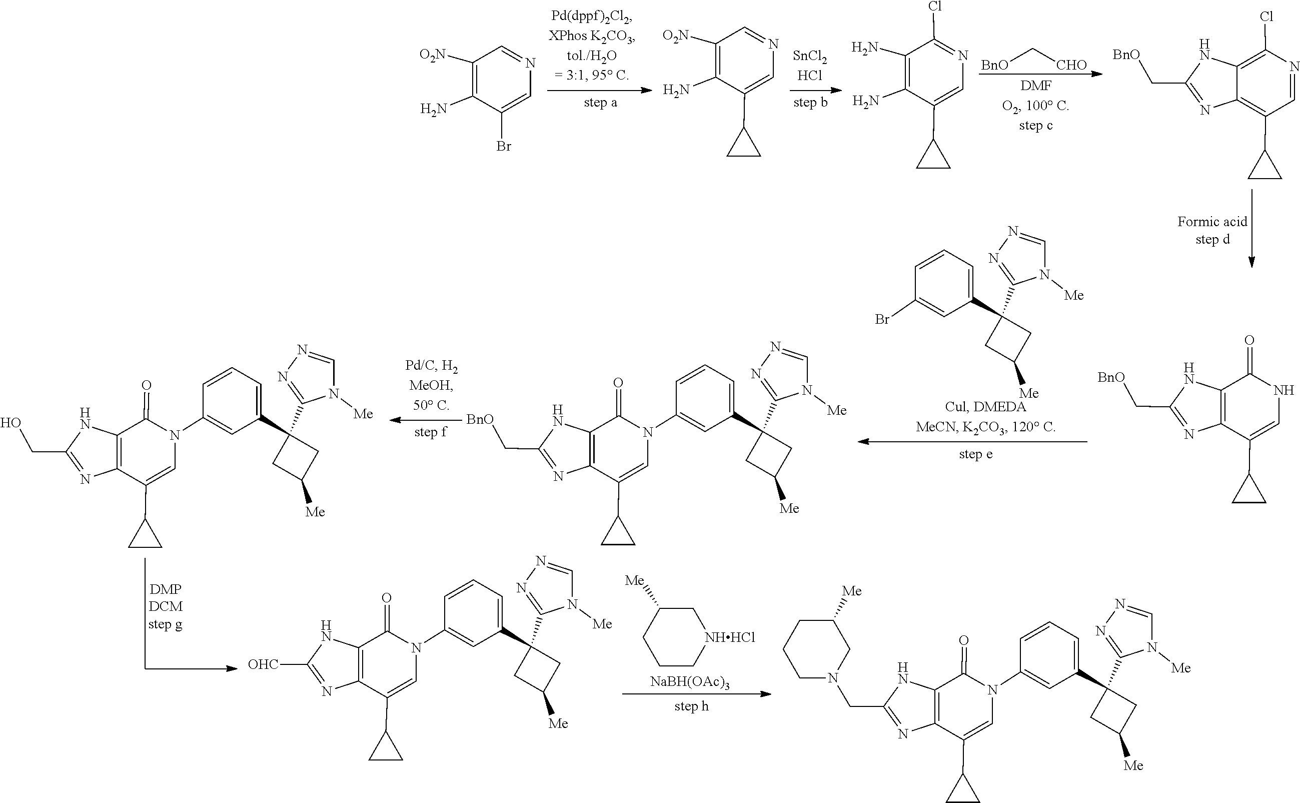

Step a: 3-Bromo-5-nitro-4-pyridinamine (218 mg, 1.0 mmol), cyclopropyl-boronic acid (129 mg, 1.5 mmol), Xphos (38.0 mg, 0.08 mmol) and K₂CO₃ (414 mg, 3.0 mmol) were dissolved in a mixture of 8 mL toluene and 2 mL water. Nitrogen was bubbled in for 10 mins followed by adding Pd(dppf)Cl₂ (36.6 mg, 0.05 mmol) and the mixture was stirred at 95° C. overnight. After cooling to room temperature, the reaction was concentrated to dryness. Purification by column chromatography (SiO₂, 0-80% EtOAc/hexanes) afforded the desired product.

Step b: To the solution of the product from step a (156 mg, 0.87 mmol) in concentrated HCl (3 mL) was heated to 90° C. and SnCl₂ (658 mg, 3.5 mmol) in 1.5 mL concentrated HCl was added dropwise. After completion of adding the SnCl₂ solution, the reaction was sealed and heated to 130° C. for 3 hours. After cooling to room temperature, the reaction was neutralized to pH=7 by adding 2M NaOH. The solution was extracted with CHCl₃:iPrOH=3:1 and the combined organic phase was dried over Na₂SO₄, concentrated and the crude residue was purified by column chromatography (SiO₂, 40-100% EtOAc/hexanes) to give the desired product.

Step c: To a solution of the product from step b (920 mg, 5.0 mmol) in DMF (10 mL, 0.5 M) was added 2-phenylmethoxy-acetaldehyde (750 mg, 5.0 mmol). Oxygen was bubbled into the resulting mixture for 10 mins and the reaction was stirred overnight at 100° C. After cooling to room temperature, the reaction was concentrated to dryness, and used for the next step directly.

Step d: The product from step c was dissolved in 5 mL formic acid and the reaction was stirred at 90° C. overnight. After cooling to room temperature, the reaction was concentrated to dryness. Purification by column chromatography (SiO₂, 0-10% MeOH/DCM) afforded the desired product.

Step e: To a solution of the product from step d (25.1 mg, 0.085 mmol), 3-[1-(3-bromophenyl)-3-methylcyclobutyl]-4-methyl-4H-1,2,4-triazole (26.0 mg, 0.085 mmol) and DMEDA (17.6 μL, 0.17 mmol) in MeCN, K₂CO₃ (35.2 mg, 0.26 mmol) was added in one portion. Nitrogen was bubbled into the solution for 10 mins followed by addition of CuI (16.2 mg, 0.085 mmol). The reaction was stirred in sealed vial at 100° C. for 2 hours. After cooling to room temperature, saturated NH₄Cl was added, and the solution was extracted with DCM. The combined organic phase was dried over Na₂SO₄, concentrated and the crude residue was purified by column chromatography (SiO₂, 0-10% MeOH/DCM) to give the desired product.

Step f: To a solution of the product from step e (207 mg, 0.4 mmol) in 5 mL MeOH was added 40 mg Pd/C under nitrogen. Hydrogen was bubbled in for 10 mins and the reaction was stirred at 50° C. under hydrogen atmosphere overnight. After cooling to room temperature, the solution was filtered through Celite® and dried under vacuum. The crude product was carried out for the next step directly without purification. ¹H NMR (400 MHz, CDCl₃) δ 8.11-7.92 (m, 1H), 7.55-7.42 (m, 2H), 7.34 (s, 1H), 7.26-7.06 (m, 1H), 6.83 (s, 1H), 4.81 (s, 2H), 3.36-3.21 (s, 3H), 3.17 (t, J=9.1 Hz, 1H), 2.88 (s, 2H), 2.67 (d, J=6.3 Hz, 3H), 2.32 (t, J=10.1 Hz, 1H), 2.02 (s, 1H), 1.14 (d, J=4.3 Hz, 3H), 0.88 (s, 2H), 0.70 (s, 2H). ESIMS [M+H]⁺ for C₂₄H₂₆N₆O₂, calcd 431.2, found 431.2.

Step g: The crude residue (25.2 mg, 0.057 mmol) from step f was dissolved in DCM (5 mL), and DMP (36 mg, 0.086 mmol) and NaHCO₃ (14.4 mg, 0.17 mmol) were added. The reaction was stirred at room temperature overnight. The reaction was quenched with 2M Na₂S₂O₃ and saturated NaHCO₃. The solution was extracted with DCM, and the combined organic phase was dried over Na₂SO₄, concentrated and the crude residue was used for the next step directly without purification.

Step h: The crude product from step g was dissolved in 5 mL DCM, and (3s)-3-methyl-piperidine hydrochloride (9.2 mg, 0.067 mmol) and Et₃N (16 μL, 0.11 mmol) were added. The reaction was stirred at room temperature for 30 mins and NaBH(OAc)₃ (18 mg, 0.086 mmol) was added, and stirred for another 1 hour. Water was added and the solution was extracted by DCM. The combined organic phase was dried over Na₂SO₄, concentrated and the crude residue was purified by prep-HPLC. ¹H NMR (400 MHz, CDCl₃) δ 7.96 (s, 1H), 7.58-7.33 (m, 4H), 7.23 (d, J=10.2 Hz, 1H), 6.86 (s, 1H), 3.80 (s, 2H), 3.27 (s, 3H), 2.96-2.75 (m, 3H), 2.68 (d, J=8.1 Hz, 2H), 2.20-1.99 (m, 2H), 1.83 (t, J=10.6 Hz, 2H), 1.78-1.54 (m, 5H), 1.14 (d, J=4.9 Hz, 3H), 0.97 (d, J=8.0 Hz, 3H), 0.87 (d, J=6.3 Hz, 3H), 0.74 (s, 2H). ESI MS [M+H]⁺ for C₃₀H₃₇N₇O, calcd 511.3, found 511.4.

### Example 110: 7-cyclopropyl-5-[3-[3-methyl-1-(4-methyl-1,2,4-triazol-3-yl)cyclobutyl]phenyl]-2-[[(2R)-2-methylmorpholin-4-yl]methyl]-3H-imidazo[4,5-c]pyridin-4-one

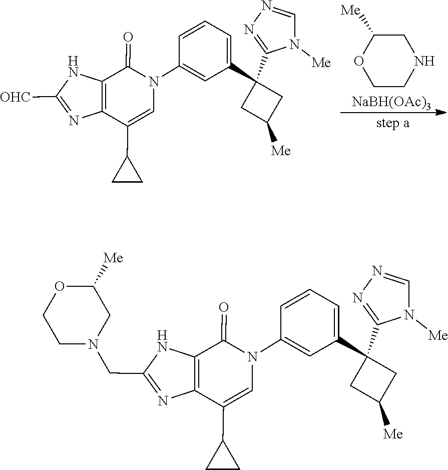

Step a: The crude product from Example 109 step g (1.0 equv.) was dissolved in 3 mL DCM, and (2R)-2-methyl-morpholine (12 mg, 0.12 mmol, 1.2 equv.) was added. The reaction was stirred at room temperature for 30 mins and NaBH(OAc)₃ (31.8 mg, 0.15 mmol, 1.5 equv.) was added, and stirred for another 1 hour. Water was added and the solution was extracted with DCM. The combined organic phase was dried over Na₂SO₄, concentrated and the crude residue was purified by prep-HPLC. 1H NMR (400 MHz, CDCl₃) δ 7.98 (s, 1H), 7.54-7.36 (m, 3H), 7.26-7.18 (m, 1H), 6.85 (s, 1H), 3.91-3.75 (m, 3H), 3.73-3.58 (m, 2H), 3.27 (s, 3H), 2.94-2.82 (m, 2H), 2.77-2.60 (m, 4H), 2.41-2.27 (m, 2H), 2.09 (s, 1H), 2.03 (t, J=10.6 Hz, 2H), 1.14 (t, J=5.7 Hz, 6H), 1.03-0.91 (m, 2H), 0.76 (d, J=5.4 Hz, 2H). ESI MS [M+H]⁺ for C₂₉H₃₅N₇O₂, calcd 514.3, found 514.4.

### Example 111: 7-cyclopropyl-2-[[(3S)-3-methylpiperidin-1-yl]methyl]-5-[3-[5-(4-methyl-1,2,4-triazol-3-yl)spiro[2.3]hexan-5-yl]phenyl]-3H-imidazo[4,5-c]pyridin-4-one

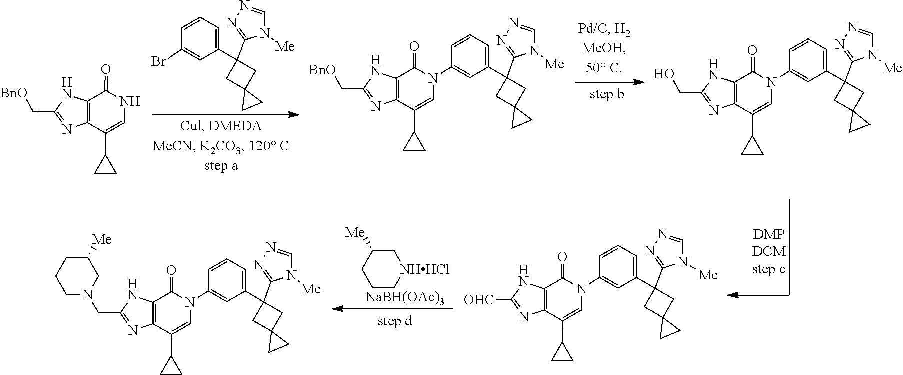

Step a: To a solution of 7-cyclopropyl-2-(phenylmethoxymethyl)-3,5-dihydroimidazo[4,5-c]pyridin-4-one (295 mg, 1.0 mmol), 3-[5-(3-bromophenyl)spiro[2.3]hex-5-yl]-4-methyl-4H-1,2,4-triazole (380 mg, 1.2 mmol), DMEDA (130 μL, 1.2 mmol) in 5 mL MeCN, and K₂CO₃ (414 mg, 3.0 mmol) were added in one portion. Nitrogen was bubbled into the solution for 10 mins followed by adding CuI (190 mg, 1.0 mmol). The reaction was stirred in sealed vial at 100° C. for 2 hours. After cooling to room temperature, saturated NH₄Cl was added, and the solution was extracted with DCM. The combined organic phase was dried over Na₂SO₄, concentrated and the crude residue was purified by column chromatography (SiO₂, 0-10% MeOH/DCM) to give the desired product.

Step b: To a solution of the product from step a (70 mg g, 0.13 mmol) in 2 mL MeOH was added 14 mg Pd/C under nitrogen. Hydrogen was bubbled in for 10 mins and the reaction was stirred at 60° C. under hydrogen atmosphere overnight. After cooling to room temperature, the solution was filtered through Celite® and dried under vacuum. The crude product was carried out for the next step directly without purification.

Step c: The crude residue (59.6 mg, 0.13 mmol) from step b was dissolved in DCM (1 mL), and DMP (82.7 mg, 0.20 mmol) and NaHCO₃ (32.8 mg, 0.39 mmol) were added. The reaction was stirred at room temperature overnight. The reaction was quenched with 2M Na₂S₂O₃ and saturated NaHCO₃. The solution was extracted with DCM, and the combined organic phase was dried over Na₂SO₄, concentrated and the crude residue was used for the next step directly without purification.

Step d: The crude product from step c (19.8 mg, 0.045 mmol) was dissolved in 1 mL DCM, and (3S)-3-methyl-piperidine hydrochloride (7.3 mg, 0.054 mmol) and Et₃N (19 ptL, 0.14 mmol) were added. The reaction was stirred at room temperature for 30 mins and NaBH(OAc)₃ (18 mg, 0.086 mmol) was added, and stir for another 1 hour. Water was added and the solution was extracted with DCM. The combined organic phase was dried over Na₂SO₄, concentrated and the crude residue was purified by prep-HPLC. ¹H NMR (400 MHz, CDCl₃) δ 8.06 (s, 1H), 7.58-7.37 (m, 2H), 7.25-7.16 (m, 2H), 6.86 (s, 1H), 3.85 (s, 2H), 3.37-3.24 (m, 5H), 2.87 (d, J=15.1 Hz, 2H), 2.82-2.74 (m, 2H), 2.23-1.97 (m, 3H), 1.87 (s, 1H), 1.80-1.54 (m, 4H), 1.01-0.91 (m, 3H), 0.87 (d, J=6.4 Hz, 3H), 0.72 (d, J=5.4 Hz, 2H), 0.65-0.49 (m, 4H). ESI MS [M+H]⁺ for C₃₁H₃₇N₇O₂, calcd 524.3, found 524.4.

### Example 112: 7-cyclopropyl-5-[3-[3,3-difluoro-1-(4-methyl-1,2,4-triazol-3-yl)cyclobutyl]phenyl]-2-[[(3S)-3-methylpiperidin-1-yl]methyl]-3H-imidazo[4,5-c]pyridin-4-one

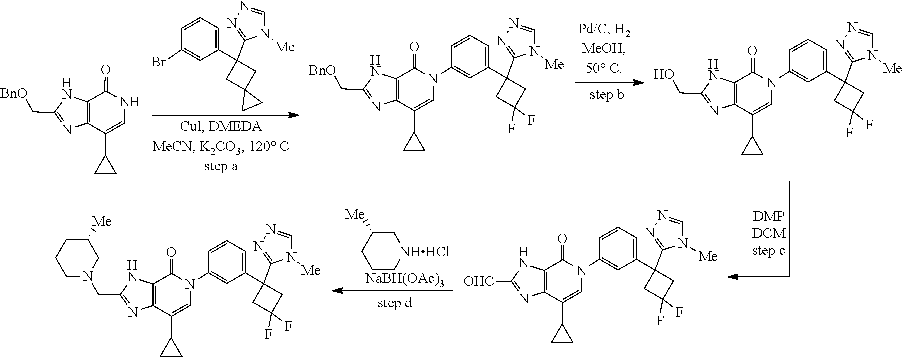

Step a: To a solution of 7-cyclopropyl-2-(phenylmethoxymethyl)-3,5-dihydroimidazo[4,5-c]pyridin-4-one (8.8 mg, 0.03 mmol), 3-[1-(3-bromophenyl)-3,3-difluorocyclobutyl]-4-methyl-4H-1,2,4-triazole (14.7 mg, 0.045 mmol), DMEDA (10 μL, 0.09 mmol) in 0.5 mL MeCN, and K₂CO₃ (12.4 mg, 0.09 mmol) were added in one portion. Nitrogen was bubbled into the solution for 10 mins followed by the addition of CuI (5.7 mg, 0.03 mmol). The reaction was stirred in a sealed vial at 100° C. for 2 hours. After cooling to room temperature, saturated NH₄Cl was added, and the solution was extracted with DCM. The combined organic phase was dried over Na₂SO₄, concentrated and the crude residue was purified by column chromatography (SiO₂, 0-10% MeOH/DCM) to give the desired product.

Step b: To a solution of the product from step a in 1 mL MeOH was added 3 mg Pd/C under nitrogen. Hydrogen was bubbled in for 10 mins and the reaction was stirred at 60° C. under hydrogen atmosphere overnight. After cooling to room temperature, the solution was filtered through Celite® and dried under vacuum. The crude product was carried used in the next step directly without purification.

Step c: The crude residue from step b was dissolved in DCM (1 mL), and DMP (15 mg, 0.036 mmol) and NaHCO₃ (7.6 mg, 0.09 mmol) were added. The reaction was stirred at room temperature overnight. The reaction was quenched with 2M Na₂S₂O₃ and saturated NaHCO₃. The solution was extracted with DCM, and the combined organic phase was dried over Na₂SO₄, concentrated and the crude residue was used in the next step directly without purification.

Step d: The crude product from step c was dissolved in 1 mL DCM, and (3S)-3-methyl-piperidine hydrochloride (4.9 mg, 0.036 mmol) and Et₃N (13 μL, 0.09 mmol) were added. The reaction was stirred at room temperature for 30 mins and NaBH(OAc)₃ (9.5 mg, 0.045 mmol) was added, and stir for another 1 hour. Water was added and the solution was extracted with DCM. The combined organic phase was dried over Na₂SO₄, concentrated and the crude residue was purified by prep-HPLC. ¹H NMR (400 MHz, CDCl₃) δ 8.06 (s, 1H), 7.52 (t, J=7.9 Hz, 1H), 7.36 (ddd, J=8.0, 2.0, 1.0 Hz, 1H), 7.30 (dt, J=5.9, 1.8 Hz, 2H), 6.86 (s, 1H), 3.86 (s, 2H), 3.77 (q, J=13.3 Hz, 2H), 3.45-3.28 (m, 5H), 2.92-2.79 (m, 2H), 2.22-2.12 (m, 2H), 2.09-2.00 (m, 2H), 1.94-1.82 (m, 2H), 1.81-1.64 (m, 2H), 1.03-0.93 (m, 3H), 0.88 (d, J=6.4 Hz, 3H), 0.73 (d, J=5.4 Hz, 2H). ESI MS [M+H]⁺ for C₂₉H₃₃F₂N₇O, calcd 534.3, found 534.4.

### Example 113: 5-[3-[3,3-difluoro-1-(4-methyl-1,2,4-triazol-3-yl)cyclobutyl]phenyl]-2-[[(3S)-3-methylpiperidin-1-yl]methyl]-7-(trifluoromethyl)-3H-imidazo[4,5-c]pyridin-4-one

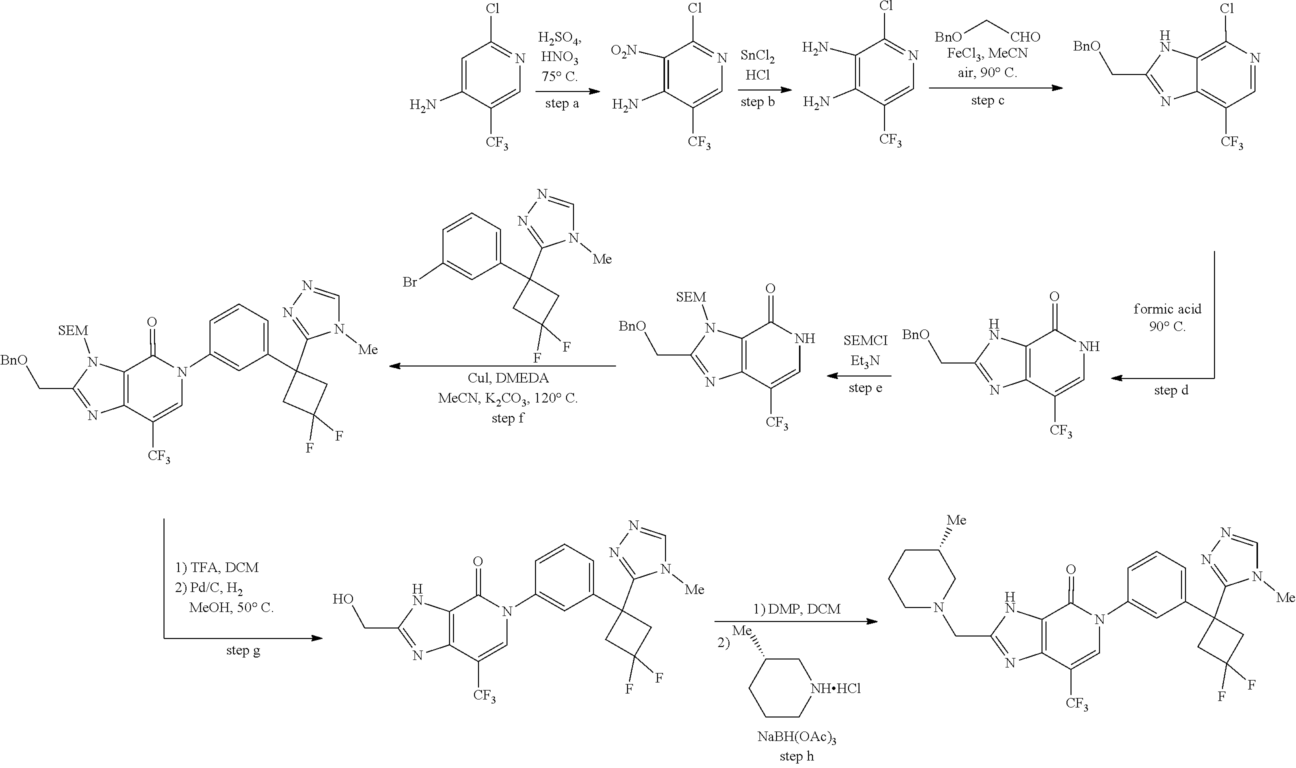

Step a: 2-Chloro-5-(trifluoromethyl)-4-pyridinamine (1.0 g, 5.1 mmol) was dissolved in 6 mL H₂SO₄ and 2 mL HNO₃ was added dropwise to the solution. The reaction was heated to 75° C. and was stirred at this temperature for 4 hours. After cooling to room temperature, the solution was poured onto ice and the solution was neutralized using 2M NaOH. The product was extracted by CHCl₃:iPrOH=3:1 and the combined organic phase was dried over Na₂SO₄, concentrated and the crude residue was purified by column chromatography (SiO₂, 0-10% EtOAc/hexanes) to afford the desired product.

Step b: To the solution of the product from step a (92 mg, 0.38 mmol) in concentrated HCl (2 mL), SnCl₂ (289 mg, 1.5 mmol) in 1 mL concentrated HCl was added dropwise. After complete addition of the SnCl₂ solution, the reaction was sealed and heated to 50° C. for 1 hours. After cooling to room temperature, the reaction was neutralized to pH=7 by adding 2M NaOH. The solution was extracted with DCM:iPrOH=3:1 and the combined organic phase was dried over Na₂SO₄, concentrated, and the crude residue was purified by column chromatography (SiO₂, 0-40% EtOAc/hexanes) to give the desired product.

Step c: To a solution of the product from step b (260 mg, 1.7 mmol) in MeCN (10 mL, 0.17 M), 2-phenylmethoxy-acetaldehyde (300 mg, 2.0 mmol) was added, and the reaction was stirred at 50° C. overnight. FeCl₃ (275 mg, 1.7 mmol) was added and air was bubbled into the resulting mixture for 10 mins. The reaction was stirred at 75° C. overnight. After cooling to room temperature, the reaction was concentrated to dryness, and was purified by column chromatography (SiO₂, 0-60% EtOAc/hexanes) to give the desired product.

Step d: The product from step c (183 mg, 0.57 mmol) was dissolved in 5 mL formic acid and the reaction was stirred at 90° C. overnight. After cooling to room temperature, the reaction was concentrated to dryness. Purification by column chromatography (SiO₂, 0-10% MeOH/DCM) afforded the desired product.

Step e: To a solution of the product from step d (44 mg, 0.14 mmol) in 2 mL DMF, Et₃N (58 μL, 0.42 mmol) was added followed by the addition of SEMCl (30 μL, 0.17 mmol), and the reaction was stirred at 70° C. for 1 hour. After cooling to room temperature, the reaction was quenched with water and extracted by EtOAc. The combined organic phase was dried over Na₂SO₄, concentrated and the crude residue was purified by column chromatography (SiO₂, 0-40% EtOAc/hexanes) to give the desired product.

Step f: To a solution of the product from step e (45 mg, 0.1 mmol), 3-[1-(3-bromophenyl)-3,3-difluorocyclobutyl]-4-methyl-4H-1,2,4-triazole (65.4 mg, 0.2 mmol), DMEDA (17.6 μL, 0.2 mmol) in 2 mL MeCN, and K₂CO₃ (41.4 mg, 0.3 mmol) was added in one portion. Nitrogen was bubbled into the solution for 10 mins followed by adding CuI (19 mg, 0.1 mmol). The reaction was stirred in sealed vial at 100° C. for 2 hours. After cooling to room temperature, saturated NH₄Cl was added, and the solution was extracted with DCM. The combined organic phase was dried over Na₂SO₄, concentrated, and the crude residue was purified by column chromatography (SiO₂, 0-10% MeOH/DCM) to give the desired product.

Step g: To a solution of the product from step f (17 mg, 0.024 mmol) in 1 mL DCM, 1 mL of TFA was added dropwise. The reaction was stirred for 1 hour and concentrated under vacuum. The crude product was dissolved in 1 mL NH₃ in MeOH and stirred for another 1 hour at room temperature before the crude mixture was concentrated. The crude product was dissolved in 2 mL MeOH and 3.4 mg Pd/C was added under nitrogen. Hydrogen was bubbled in the reaction mixture for 10 mins and the reaction was stirred at room temperature under hydrogen atmosphere overnight. After cooling to room temperature, the solution was filtered through Celite® and dried under vacuum. The crude product was carried used in the next step directly without purification.

Step h: The crude residue from step g was dissolved in DCM (0.5 mL), and DMP (15.3 mg, 0.036 mmol) and NaHCO₃ (6.0 mg, 0.72 mmol) were added. The reaction was stirred at room temperature for 1 hour. The reaction was quenched with 2M Na₂S₂O₃ and saturated NaHCO₃. The solution was extracted with DCM, and the combined organic phase was dried over Na₂SO₄, concentrated, and the crude residue was used in the next step directly without purification. The crude product was dissolved in 1 mL DCM, and (3S)-3-methyl-piperidine hydrochloride (3.9 mg, 0.029 mmol) and Et₃N (8 μL, 0.058 mmol) were added. The reaction was stirred at room temperature for 30 mins and NaBH(OAc)₃ (7.6 mg, 0.036 mmol) was added, and stirred for another 1 hour. Water was added and the solution was extracted with DCM. The combined organic phase was dried over Na₂SO₄, concentrated, and the crude residue was purified by prep-HPLC. ¹H NMR (400 MHz, CDCl₃) δ 8.07 (s, 1H), 7.63-7.54 (m, 2H), 7.46-7.40 in, 1H), 7.34-7.28 (m, 2H), 3.90 (s, 2H), 3.78 (q, J=13.2 Hz, 2H), 3.43-7.28 (m, 5H), 2.92-2.78 (m, 3H), 2.29-2.12 (m, 2H), 1.93 (t, J=10.8 Hz, 1H), 1.82-1.59 (m, 4H), 0.89 (d, J=6.4 Hz, 3H). ESI MS [M+H]⁺ for C₂₇H₂₈F₅N₇O, calcd 562.3, found 562.3.

### Example 114: 5-[3-[3,3-difluoro-1-(4-methyl-1,2,4-triazol-3-yl)cyclobutyl]phenyl]-2-[[(2R)-2-methylmorpholin-4-yl]methyl]-7-(trifluoromethyl)-3H-imidazo[4,5-c]pyridin-4-one

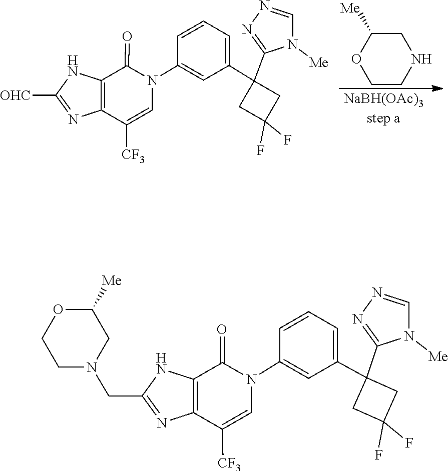

Step a: 5-[3-[3,3-Difluoro-1-(4-methyl-1,2,4-triazol-3-yl)cyclobutyl]phenyl]-4-oxo-7-(trifluoromethyl)-3H-imidazo[4,5-c]pyridine-2-carbaldehyde (14.3 mg, 0.03 mmol, obtained according to example 113, step 1 of step h) was dissolved in 0.5 mL DCM, and (2R)-2-methyl-morpholine (3.6 mg, 0.036 mmol) was added. The reaction was stirred at room temperature for 30 mins and NaBH(OAc)₃ (9.5 mg, 0.045 mmol) was added, and stirred for another 1 hour. Water was added and the solution was extracted with DCM. The combined organic phase was dried over Na₂SO₄, concentrated, and the crude residue was purified by prep-HPLC. ¹H NMR (400 MHz, CDCl₃) δ 8.08 (s, 1H), 7.62-7.55 (m, 2H), 7.48-7.43 (m, 1H), 7.34-7.27 (m, 2H), 3.96-3.83 (m, 2H), 3.81-3.60 (m, 4H), 3.45-3.26 (m, 5H), 2.77-2.65 (m, 2H), 2.41 (td, J=11.4, 3.2 Hz, 1H), 2.09 (t, J=10.6 Hz, 1H), 1.66 (s, 2H), 1.16 (d, J=6.3 Hz, 3H). ESI MS [M+H]⁺ for C₂₆H₂₆F₅N₇O, calcd 564.3, found 564.3.

### Example 115: 5-[3-[3,3-difluoro-1-(4-methyl-1,2,4-triazol-3-yl)cyclobutyl]phenyl]-2-[(3,3-difluoropyrrolidin-1-yl)methyl]-7-(trifluoromethyl)-3H-imidazo[4,5-c]pyridin-4-one

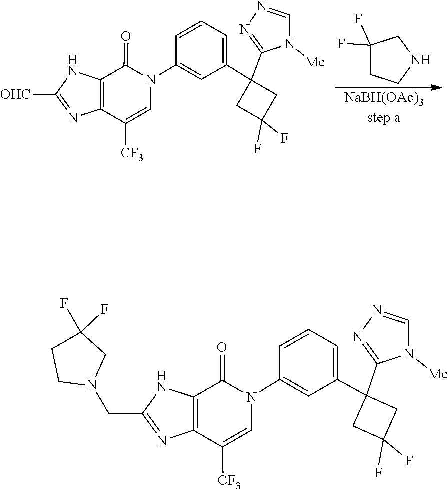

Step a: 5-[3-[3,3-difluoro-1-(4-methyl-1,2,4-triazol-3-yl)cyclobutyl]phenyl]-4-oxo-7-(trifluoromethyl)-3H-imidazo[4,5-c]pyridine-2-carbaldehyde (14.3 mg, 0.03 mmol, obtained according to example 113, step 1 of step h) was dissolved in 0.5 mL DCM, and 3,3-difluoro-pyrrolidine (3.8 mg, 0.036 mmol) was added. The reaction was stirred at room temperature for 30 mins and NaBH(OAc)₃ (9.5 mg, 0.045 mmol) was added, and stirred for another 1 hour. Water was added and the solution was extracted with DCM. The combined organic phase was dried over Na₂SO₄, concentrated, and the crude residue was purified by prep-HPLC. 1H NMR (400 MHz, CDCl₃) δ 8.10 (d, J=2.1 Hz, 1H), 7.64-7.55 (m, 2H), 7.51-7.42 (m, 2H), 7.34-7.28 (m, 1H), 4.05 (s, 2H), 3.86-3.68 (m, 2H), 3.37 (s, 5H), 3.22 (s, 1H), 3.09 (t, J=12.8 Hz, 1H), 2.93 (t, J=7.0 Hz, 2H), 2.37 (tt, J=14.4, 7.0 Hz, 2H), 1.88 (s, 1H). ESI MS [M+H]⁺ for C₂₅H₂₂F₇N₇₀, calcd 570.2, found 570.2.

### Example 116: 4-cyclopropyl-6-{3-[3-methyl-1-(4-methyl-4H-1,2,4-triazol-3-yl)cyclobutyl]phenyl}-1H,6H,7H-pyrrolo[2,3-d]pyridazin-7-one

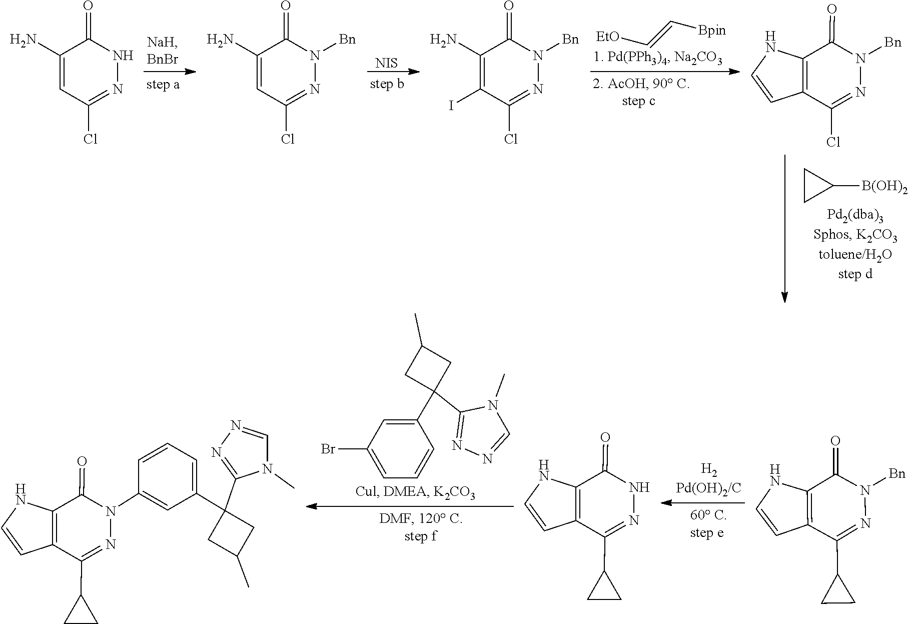

Step a: To a solution of 5-amino-3-chloro-1H-pyridazin-6-one (500 mg, 3.4350 mmol, 1.0 equiv.) in THE (10 ml, 0.3 M) was added NaH (124 mg, 5.1525 mmol, 1.5 equiv.) and BnBr (0.41 ml, 3.4350 mmol, 1.0 equiv.) at 0° C. The resulting mixture was stirred at rt for 2 h. The reaction mixture was quenched with H₂O, the organic phase was separated, and the aqueous layer was extracted with EtOAc. The combined organic phase was dried over Na₂SO₄, concentrated, and the crude residue was purified by column chromatography (SiO₂, EtOAc in hexanes, 20 to 80%) to give 4-amino-2-benzyl-6-chloropyridazin-3-one.

Step b: To a solution of the product from step a (380 mg, 1.479 mmol, 1.0 equiv.) in CH₃CN (5 ml, 0.3 M) was added N-iodosuccinimide (478 mg, 2.2169 mmol, 1.5 equiv.). The resulting mixture was heated to reflux overnight. After cooling down to rt, the reaction mixture was quenched with H₂O, the organic phase was separated, and the aqueous layer was extracted with EtOAc. The combined organic phase was dried over Na₂SO₄, concentrated, and the crude residue was purified by column chromatography (SiO₂, EtOAc in hexanes, 20 to 80%) to give 4-amino-2-benzyl-6-chloro-5-iodopyridazin-3-one.

Step c: To solution of the product from step b (326 mg, 0.9055 mmol, 1.0 equiv.) and (E)-2-(2-ethoxyvinyl)-4,4,5,5-tetramethyl-1,3,2-dioxaborolane in toluene/EtOH/H₂O (4 mL/1 ml/lml) was added Na₂CO₃ (101 mg, 1.8111 mmol, 2.0 equiv.) at rt. To the resulting mixture was added Pd(PPh₃)₄ (152 mg, 0.04528 mmol, 5% equiv.). The resulting mixture was stirred at 90° C. for 12 h. The reaction was quenched with sat. aq. NH₄Cl solution, the organic phase was separated, and the aqueous layer was extracted with EtOAc. The combined organic phase was dried over Na₂SO₄ and concentrated. The crude residue was dissolved in AcOH (10 ml) and stirred at 90° C. for 12 h. After cooling to rt, the reaction mixture was quenched with H₂O, the organic phase was separated, and the aqueous layer was extracted with EtOAc. The combined organic phase was dried over Na₂SO₄, concentrated, and the crude residue was purified by column chromatography (SiO₂, EtOAc in hexanes, 20 to 80%) to give 6-benzyl-4-cyclopropyl-1H-pyrrolo[2,3-d]pyridazin-7-one.

Step d: The product of step c (45 mg, 0.1731 mmol, 1.0 equiv.), cyclopropylboronic acid (23 mg, 0.2596 mmol, 1.5 equiv.) and K₂PO₄ (110 mg, 0.5193 mmol, 3.0 equiv.) were dissolved in toluene/H₂O (4 mL/1 mL, 0.03 M). The mixture was purged for 2 mins under N₂. Then, Pd₂(dba)₃ (8 mg, 0.008655 mmol, 0.05 equiv.) and Sphos (7 mg, 0.01731 mmol, 0.1 equiv.) were added into the solution. The mixture was stirred at 110° C. for 12 h. After cooling to rt, the reaction mixture was quenched with H₂O, the organic phase was separated, and the aqueous layer was extracted with EtOAc. The combined organic phase was dried over Na₂SO₄, concentrated, and the crude residue was purified by column chromatography (SiO₂, EtOAc in hexanes, 20 to 80%) to afford 6-benzyl-4-cyclopropyl-1H-pyrrolo[2,3-d]210yridazine-7-one.

Step e: To a solution of the product from step d (45 mg, 0.1698 mmol, 1.0 equiv.) in AcOH (3 mL, 0.05 M) was added Pd(OH)₂/C (12 mg, 0.01698 mmol, 0.1 equiv., 20% wt) at rt under H₂ atmosphere. The resulting mixture was stirred at 60° C. for 2 h. The organic phase was filtered, and the organic phase was combined and concentrated and the crude residue was purified by column chromatography (SiO₂, MeOH in DCM, 0 to 10%) to give 4-cyclopropyl-1,6-dihydropyrrolo[2,3-d]pyridazin-7-one.

Step f: To a solution of the product from step e (13 mg, 0.07386 mmol, 1.0 equiv.) and 3-[1-(3-bromophenyl)-3-methylcyclobutyl]-4-methyl-1,2,4-triazole (34 mg, 0.1108 mmol, 1.5 equiv.) in dioxane (5 mL, 0.015 M) was added CuI (14 mg, 0.07386 mmol, 1.0 equiv.), DMEDA (26 mg, 0.2954 mmol, 4.0 equiv.) and K₂CO₃ (31 mg, 0.2216 mmol, 3.0 equiv.). The resulting solution was stirred at 110° C. for 2 h. The reaction was quenched with H₂O, the organic phase was separated, and the aqueous layer was extracted with EtOAc. The combined organic phase was dried over Na₂SO₄, concentrated, and the crude residue was purified by column chromatography (MeOH in DCM, 0 to 20%) to give a crude mixture. The resulting residue was further was purified by prep-HPLC to furnish the title compound. ¹H NMR (400 MHz, CDCl₃) δ 10.02 (s, 1H), 7.96 (s, 1H), 7.71 (s, 1H), 7.53 (d, J=7.7 Hz, 1H), 7.43 (t, J=7.9 Hz, 1H), 7.32 (d, J=14.0 Hz, 2H), 6.64 (s, 1H), 3.26 (s, 3H), 2.89 (s, 2H), 2.67 (d, J=6.3 Hz, 2H), 2.18 (s, 1H), 1.17-1.09 (m, 5H), 1.01 (d, J=7.9 Hz, 3H). ESI MS [M+H]⁺ for C₂₃H₂₅N₆O, calcd 401.5, found 401.1.

### Example 117 and 118: 7-cyclopropyl-5-[3-[cis-3-methyl-1-(4-methyl-1,2,4-triazol-3-yl)cyclobutyl]phenyl]-1,3-dihydroimidazo[4,5-c]pyridine-2,4-dione and 7-cyclopropyl-5-[3-[trans-3-methyl-1-(4-methyl-1,2,4-triazol-3-yl)cyclobutyl]phenyl]-1,3-dihydroimidazo[4,5-c]pyridine-2,4-dione

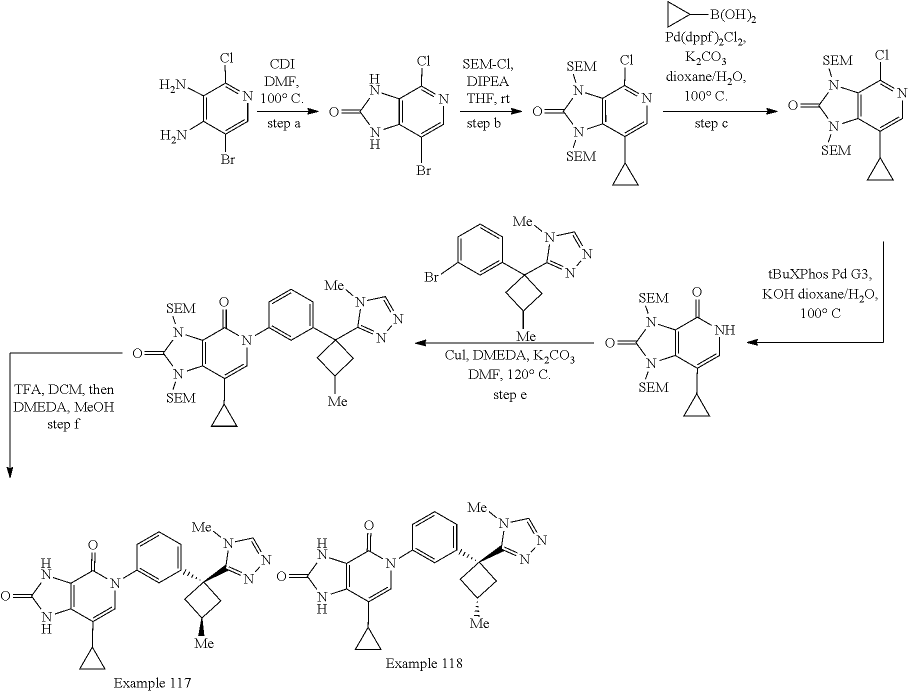

Step a: To a solution of 5-bromo-2-chloropyridine-3,4-diamine (320 mg, 1.44 mmol, 1.0 equiv.) in DMF (2.9 mL) was added CDI (245 mg, 1.51 mmol, 1.05 equiv.). The reaction mixture was heated to 100° C. and stirred for 16 hours at which point it was cooled to room temperature and diluted with water (30 mL). The precipitate was collected via filtration and dried under vacuum.

Step b: To a solution of the product from Step a (200 mg, 0.81 mmol, 1.0 equiv.) in THF (8 mL) was added DIPEA (0.56 mL, 3.22 mmol, 4.0 equiv.) followed by SEM-Cl (0.57 mL, 3.22 mL, 4.0 equiv.). The reaction mixture was stirred for 16 hours at room temperature at which point it was quenched with water (100 mL) and extracted with EtOAc (2×60 mL). The combined organics were washed with saturated aqueous NaCl, dried over Na₂SO₄, filtered, and concentrated under vacuum. The crude residue was purified via silica gel flash column chromatography (0 to 40% EtOAc/hexanes) to afford the desired product.

Step c: A solution of the product from Step b (150 mg, 0.39 mmol, 1.0 equiv.), cyclopropylboronic acid (38 mg, 0.44 mmol, 1.5 equiv.), K₂CO₃ (122 mg, 0.88 mmol, 3.0 equiv.) in a 3:1 mixture of dioxane/water (3 mL) was sparged with N₂ for 10 minutes. Pd(dppf)₂Cl₂ (22 mg, 0.03 mmol, 0.1 equiv.) was added and the reaction was heated to 100° C. and stirred for 1 hour. The reaction was quenched with a ˜1:1 mixture of saturated aqueous NaCl/water (30 mL) and extracted with EtOAc (2×20 mL). The combined organics were dried over Na₂SO₄, filtered, and concentrated under vacuum. The crude residue was purified via silica gel flash column chromatography (0 to 40% EtOAc/hexanes) to afford the desired product.

Step d: To a solution of the product from Step c (110 mg, 0.23 mmol, 1.0 equiv.) in dioxane (2.3 mL) was added 3M aqueous KOH (0.8 mL, 2.3 mmol, 10.0 equiv.). The reaction mixture was sparged with N₂ for 10 minutes and tBuXPhos Pd G3 (40 mg, 0.05 mmol, 0.2 equiv.) was added. The reaction was heated to 100° C. and stirred for 30 minutes at which point it was quenched with a ˜1:1 mixture of saturated aqueous NaCl/water (20 mL) and extracted with a 3:1 mixture of CHC13/iPrOH (2×15 mL). The combined organics were dried over Na₂SO₄, filtered, and concentrated under vacuum. The crude residue was purified via silica gel flash column chromatography (0 to 100% EtOAc/DCM) to afford the desired product.

Step e: To a solution of the product from Step d (50 mg, 0.11 mmol, 1.0 equiv.), 3-[1-(3-bromophenyl)-3-methylcyclobutyl]-4-methyl-1,2,4-triazole (52 mg, 0.17 mmol, 1.5 equiv.), and K₂CO₃ (46 mg, 0.33 mmol, 3.0 equiv.) in DMF (1.5 mL) was added CuI (21 mg, 0.11 mmol, 1.0 equiv.) followed by DMEDA (24 uL, 0.22 mmol, 2.0 equiv.). The reaction mixture was heated to 120° C. and stirred for 1 hour at which point it was quenched with a ˜1:1 mixture of saturated aqueous NaCl/water (15 mL) and extracted with EtOAc (2×10 mL). The combined organics were dried over Na₂SO₄, filtered, and concentrated under vacuum. The crude residue was purified via silica gel flash column chromatography (0 to 20% MeOH/DCM) to afford the desired product as a mixture of diastereomers.

Step f: A solution of the product from Step e (50 mg, 0.07 mmol, 1.0 equiv.) in a 1:1 mixture of TFA/DCM (1 mL) was stirred at room temperature for 1 hour. Toluene (5 mL) was added and the reaction mixture was directly concentrated under vacuum. The crude residue was dissolved in MeOH (1 mL) and DMEDA (0.25 mL) was added. The reaction mixture was heated to 45° C. and stirred for 30 minutes at which point it was directly concentrated under vacuum and the crude residue was purified via RP-HPLC (10 to 45% MeCN/H₂O) to afford two separable diastereomeric products.

Example 117 (first-eluting diastereomer, ˜20:1 dr): ¹H NMR (400 MHz, CDCl₃) δ 9.22 (br s, 1H), 8.42 (br s, 1H), 7.97 (s, 1H), 7.56-7.40 (m, 2H), 7.34 (s, 1H), 7.25-7.18 (m, 1H), 6.93 (s, 1H), 3.25 (s, 3H), 2.88 (d, J=9.6 Hz, 2H), 2.76-2.60 (m, 3H), 1.78-1.66 (m, 1H), 1.14 (d, J=5.4 Hz, 3H), 0.88 (d, J=8.0 Hz, 2H), 0.58 (d, J=5.4 Hz, 2H). ESI MS [M+H]⁺ for C₂₃H₂₄N₆O₂, calcd 417.2, found 417.2.

Example 118 (second-eluting diastereomer, ˜2.5:1 dr): ¹H NMAIR (400 MHz, CDCl₃) δ 8.03 (s, 1H), 7.49-7.41 (m, 1H), 7.31 (t, J=7.9 Hz, 1H), 7.24-7.15 (m, 2H), 6.94 (s, 1H), 3.31 (s, 3H), 3.19 (t, J=9.3 Hz, 1H), 2.93-2.52 (m, 3H), 2.30 (t, J=10.1 Hz, 1H), 1.76-1.65 (m, 1H), 1.13 (d, J=6.5 Hz, 3H), 0.90 (d, J=8.1 Hz, 2H), 0.59 (d, J=5.4 Hz, 2H). ESI MS [M+H]⁺ for C₂₃H₂₄N₆O₂, calcd 417.2, found 417.2.

### Example 119: 6—{[(3S)-3-Methylpiperidin-1-yl]methyl}-3-{3-[(1R,3S)-3-methyl-1-(4-methyl-4H-1,2,4-triazol-3-yl)cyclobutyl]phenyl}-3H,4H,5H-pyrrolo[3,2-d]pyrimidin-4-one

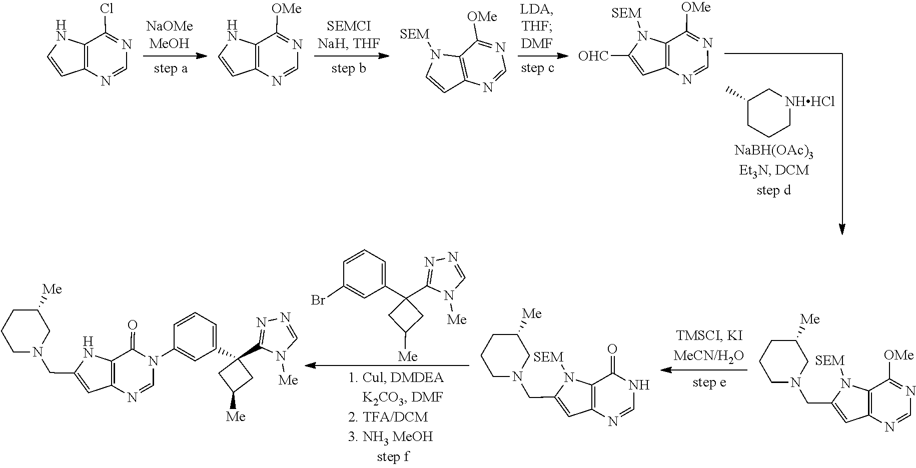

Step a: To a solution of 4-chloro-5H-pyrrolo[3,2-d]pyrimidine (1.00 g, 6.5 mmol, 1.0 equiv.) in methanol (20 mL) was added sodium methoxide (540 mg, 10 mmol, 1.5 equiv.). The resulting mixture was heated at 60° C. overnight, and then concentrated on Celite®. The residue was purified by column chromatography (SiO₂, MeOH in DCM, 0 to 10%) to give the product.

Step b: To a solution of the product from step a (500 mg, 3.4 mmol, 1.0 equiv.) in THE (7 mL) was added NaH (60 wt % in mineral oil, 148 mg, 3.7 mmol, 1.1 equiv.) at 0° C. The resulting mixture was stirred at this temperature for 10 min before the addition of 2-(trimethylsilyl)ethoxymethyl chloride (737 mg, 0.78 mL, 4.4 mmol, 1.3 equiv.). The reaction mixture was then raised to room temperature and stirred overnight, before quenched with water and diluted with EtOAc. The organic phase was separated, washed with brine, dried over Na₂SO₄, and concentrated. The crude residue was purified by column chromatography (SiO₂, EtOAc in hexanes, 0 to 40%) to give the desired product.

Step c: To a solution of the product from step b (279 mg, 1.0 mmol, 1.0 equiv.) in THE (4 mL) was added lithium diisopropylamide (2M in THF/heptane/ethylbenzene, 0.55 mL, 1.1 mmol, 1.1 equiv.) at −78° C. The resulting solution was stirred at this temperature for another 30 min, and DMF (0.54 mL, 512 mg, 7.0 mmol, 5.0 equiv.) was added. After another 30-min stirring at −78° C., the reaction mixture was quenched with saturated NH₄Cl aqueous solution and warmed to room temperature. The organic phase was separated, and the aqueous phase was extracted with EtOAc twice. The combined organic solution was then washed with brine, dried over Na₂SO₄, and concentrated. The crude product was directly applied in the next step.

Step d: To a solution of the crude product from step c (˜1.0 mmol, 1.0 equiv.) in DCM (5 mL) was added (S)-3-methylpiperidine hydrochloride (203 mg, 1.5 mmol, 1.5 equiv.) and Et₃N (0.28 mL, 202 mg, 2.0 mmol, 2.0 equiv.). The resulting mixture was stirred at room temperature for 30 min before adding NaBH(OAc)₃ (424 mg, 2.0 mmol, 2.0 equiv.). The reaction mixture was then stirred at room temperature for another 45 min before being quenched with H₂O. The organic phase was separated, and the aqueous layer was extracted with DCM twice. The combined organic phase was dried over Na₂SO₄, concentrated, and the crude residue was purified by column chromatography (SiO₂, MeOH in DCM, 0 to 10%) to give 2-[[4-methoxy-6-[[(3S)-3-methylpiperidin-1-yl]methyl]pyrrolo[3,2-d]pyrimidin-5-yl]methoxy]ethyl-trimethylsilane.

Step e: To a mixture of the product from step d (358 mg, 0.92 mmol, 1.0 equiv.) in MeCN/H₂O (4.1 v/v, 4 mL) was added TMSCl (0.19 mL, 160 mg, 1.5 mmol, 1.6 equiv.) and KI (244 mg, 1.5 mmol, 1.6 equiv.). The resulting mixture was stirred at room temperature overnight when LCMS showed a completion of the demethylation. The reaction mixture was concentrated to dryness, and the crude product was purified by column chromatography (SiO₂, MeOH in DCM, 0 to 10%) to give 6-[[(3S)-3-methylpiperidin-1-yl]methyl]-5-(2-trimethylsilylethoxymethyl)-3H-pyrrolo[3,2-d]pyrimidin-4-one.

Step f: To a solution of the product from step e (75.3 mg, 0.20 mmol, 1.0 equiv.) and 3-[1-(3-bromophenyl)-3-methylcyclobutyl]-4-methyl-1,2,4-triazole (diastereomeric mixture in 3:1 ratio, 73.5 mg, 0.24 mmol, 1.2 equiv.) in DMF (2 mL) was added CuI (38.1 mg, 0.20 mmol, 1.0 equiv.), N,N′-dimethylethylenediamine (26.4 mg, 0.30 mmol, 1.5 equiv.), and K₂CO₃ (55.3 mg, 0.40 mmol, 2.0 equiv.). The resulting mixture was heated at 110° C. for 6 h. After cooling to room temperature, the reaction mixture was diluted with EtOAc and washed with water twice. The organic phase was then washed with brine, dried over Na₂SO₄ and concentrated. The residue was then treated with TFA/DCM (v/v 1:10, 4 mL) at room temperature for 4 h, and then concentrated. The residue was then treated with 7M NH₃ in methanol (4 mL) for 10 min, followed by the concentration. The crude residue was then purified by HPLC to afford Example 119 as a diastereomeric mixture in 4:1 ratio. ¹H NMR (400 MHz, DMSO-d₆) δ 12.13 (s, 1H), 8.37 (s, 0.2H), 8.29 (s, 0.8H), 8.05 (s, 0.8H), 8.04 (s, 0.2H), 7.60-7.28 (m, 4H), 6.29 (s, 1H), 3.57 (d, J=2.4 Hz, 2H), 3.29 (s, 0.6H), 3.24 (s, 2.4H), 3.12 (dd, J=10.2, 6.5 Hz, 0.4H), 2.93-2.66 (m, 4H), 2.54 (d, J=6.6 Hz, 2H), 2.37-2.23 (m, 0.6H), 1.88 (td, J=11.4, 2.7 Hz, 1H), 1.72-1.38 (m, 5H), 1.13-1.04 (m, 3H), 0.89-0.73 (m, 4H). ESI MS [M+H]⁺ for C₂₇H₃₃N₇O, calcd 472.3, found 472.3.

### Example 120: 3-{3-[3,3-Difluoro-1-(4-methyl-4H-1,2,4-triazol-3-yl)cyclobutyl]phenyl}-6-{[(3S)-3-methylpiperidin-1-yl]methyl}-3H,4H,5H-pyrrolo[3,2-d]pyrimidin-4-one

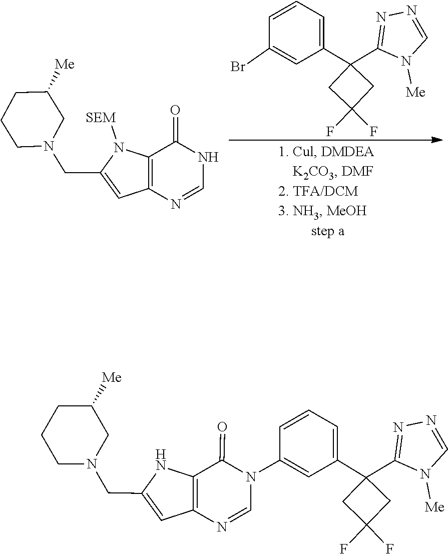 [SMILES: C[C@H]1CCCN(Cc2cc3nc[nH]c(=O)c3n2COCC[Si](C)(C)C)C1 ; Cn1cnnc1C1(c2cccc(Br)c2)CC(F)(F)C1 ; C[C@H]1CCCN(Cc2cc3ncn(-c4cccc(C5(c6nncn6C)CC(F)(F)C5)c4)c(=O)c3[nH]2)C1]

Step a: To a solution of 6-[[(3S)-3-methylpiperidin-1-yl]methyl]-5-(2-trimethylsilylethoxymethyl)-3H-pyrrolo[3,2-d]pyrimidin-4-one (81.7 mg, 0.22 mmol, 1.0 equiv.), and 3-[1-(3-bromophenyl)-3,3-difluorocyclobutyl]-4-methyl-1,2,4-triazole (85.3 mg, 0.26 mmol, 1.2 equiv.) in DMF (2 mL) was added CuI (41.9 mg, 0.22 mmol, 1.0 equiv.), N,N′-dimethylethylenediamine (29.1 mg, 0.33 mmol, 1.5 equiv.) and K₂CO₃ (60.8 mg, 0.44 mmol, 2.0 equiv.). The resulting mixture was heated at 110° C. for 6 h. After cooling to room temperature, the reaction mixture was diluted with EtOAc and washed with water twice. The organic phase was then washed with brine, dried over Na₂SO₄ and concentrated. The residue was then treated with TFA/DCM (v/v 1:10, 4 mL) at room temperature for 4 h, and then concentrated. The residue was then treated with 7M NH₃ in methanol (4 mL) for 10 min, followed by the concentration. The crude residue was then purified by HPLC to afford the title compound. ¹H NMR (400 MHz, CDCl₃) δ 11.30 (s, 1H), 8.09 (s, 1H), 7.92 (s, 1H), 7.59-7.53 (m, 1H), 7.46-7.38 (m, 1H), 7.34-7.27 (m, 2H), 6.48 (s, 1H), 4.11-3.90 (m, 2H), 3.77 (q, J=13.4 Hz, 2H), 3.45-3.27 (m, 5H), 3.11 (dd, J=40.2, 11.3 Hz, 2H), 2.39-2.27 (m, 1H), 2.12-1.71 (m, 5H), 1.01 (dt, J=11.8, 6.2 Hz, 1H), 0.91 (d, J=6.3 Hz, 3H). ESI MS [M+H]⁺ for C₂₆H₂₉F₂N₇O, calcd 494.2, found 494.2.

### Example 121: 4-cyclopropyl-6-[3-[3,3-difluoro-1-(1,3,4-oxadiazol-2-yl)cyclobutyl]phenyl]-2-[[(3S)-3-methylpiperidin-1-yl]methyl]-1H-pyrrolo[2,3-c]pyridin-7-one

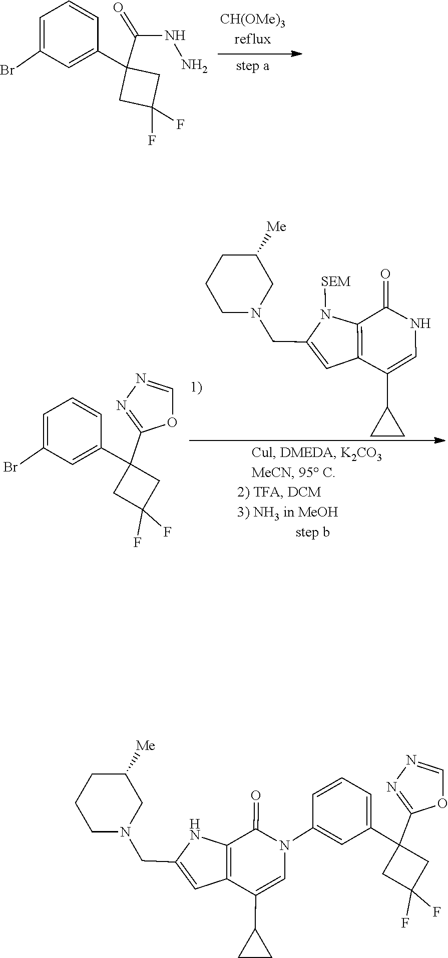 [SMILES: NNC(=O)C1(c2cccc(Br)c2)CC(F)(F)C1 ; FC1(F)CC(c2cccc(Br)c2)(c2nnco2)C1 ; C[C@H]1CCCN(Cc2cc3c(C4CC4)c[nH]c(=O)c3n2COCC[Si](C)(C)C)C1 ; C[C@H]1CCCN(Cc2cc3c(C4CC4)cn(-c4cccc(C5(c6nnco6)CC(F)(F)C5)c4)c(=O)c3[nH]2)C1]

Step a: 1-(3-bromophenyl)-3,3-difluorocyclobutane-1-carbohydrazide (30 mg, 0.1 mmol) was dissolved in 1 mL HC(OMe)₃, and pTSA (1.9 mg, 0.01 mmol) was added. The reaction was heated under reflux for 3 hours. After cooling to room temperature, the reaction was concentrated, and the crude residue was purified by column chromatography (SiO₂, 0-60% EtOAc/hexanes) to give the desired product.

Step b: To a solution of the product from step a (94.2 mg, 0.3 mmol), 4-cyclopropyl-2-[[(3S)-3-methylpiperidin-1-yl]methyl]-1-(2-trimethylsilylethoxymethyl)-6H-pyrrolo[2,3-c]pyridin-7-one (83 mg, 0.2 mmol), DMEDA (43 μL, 0.2 mmol) in 2 mL MeCN, and K₂CO₃ (83 mg, 0.6 mmol) were added in one portion. Nitrogen was bubbled into the solution for 10 mins followed by the addition of CuI (38 mg, 0.2 mmol). The reaction was stirred in sealed vial at 100° C. for 2 hours. After cooling to room temperature, saturated NH₄Cl was added, and the solution was extracted with DCM. The combined organic phase was dried over Na₂SO₄, concentrated and the crude residue was purified by column chromatography (SiO₂, 0-50% EtOAc/hexanes) to give the desired product. The product was used in the next step directly.

To a solution of crude product in 2 mL DCM, 2 mL TFA was added dropwise, and the reaction was stirred for 1 hour and then concentrated under vacuum. The crude product was dissolved in 2 mL NH₃ in MeOH and stir for another 1 hour at room temperature before the crude product was concentrated. The crude residue was purified by prep-HPLC. ¹H NMR (400 MHz, CDCl₃) δ 10.21 (s, 1H), 8.35 (s, 1H), 7.51 (t, J=7.8 Hz, 1H), 7.43 (t, J=2.0 Hz, 1H), 7.41-7.32 (m, 2H), 6.70 (d, J=1.2 Hz, 1H), 6.41 (d, J=1.5 Hz, 1H), 3.78-3.62 (m, 4H), 3.41 (dtd, J=14.7, 11.9, 3.1 Hz, 2H), 2.84 (dd, J=18.6, 10.4 Hz, 2H), 2.04-1.94 (m, 1H), 1.88 (dddd, J=10.5, 8.3, 5.3, 1.2 Hz, 1H), 1.79-1.57 (m, 5H), 0.94-0.79 (m, 6H), 0.68-0.60 (m, 2H). ESI MS [M+H]⁺ for C₂₉H₃₁F₂N₅O₂, calcd 520.2, found 520.2.

### Example 122 and 123: 4-cyclopropyl-6-[3-[3,3-difluoro-1-(triazol-2-ylmethyl)cyclobutyl]phenyl]-2-[[(3S)-3-methylpiperidin-1-yl]methyl]-1H-pyrrolo[2,3-c]pyridin-7-one and 4-cyclopropyl-6-[3-[3,3-difluoro-1-(triazol-1-ylmethyl)cyclobutyl]phenyl]-2-[[(3S)-3-methylpiperidin-1-yl]methyl]-1H-pyrrolo[2,3-c]pyridin-7-one

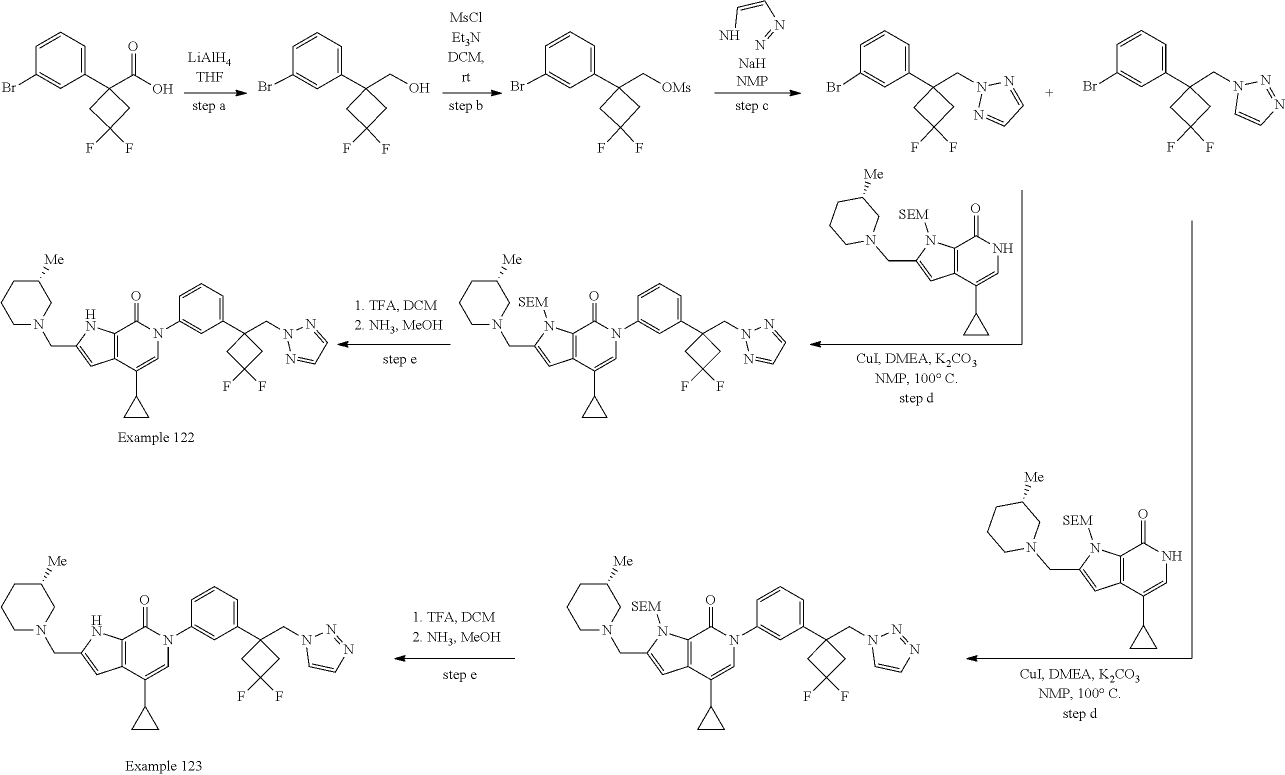

Step a: To a solution of 1-(3-bromophenyl)-3,3-difluorocyclobutane-1-carboxylic acid (4.9 g, 17 mmol) in THF (85.0 mL) was added lithium aluminum hydride (2.0 M in THF solution, 4.3 mL) slowly at 0° C. The reaction was stirred and allowed to warm to room temperature. After 2 hours, when NMR showed complete conversion, the reaction was cooled to 0° C. and worked up using the Fieser method. The reaction was quenched with water (0.65 mL), followed by the addition of 15% NaOH aq (1.94 mL), then water (1.94 mL). The resulting mixture was warmed to room temperature and stirred for 15 minutes. Anhydrous MgSO₄ was then added to the mixture and stirred for another 15 minutes. The precipitate was removed by filtration. The filtrate was concentrated to afforded the crude product.

Step b: To a solution of the crude product from step a in DCM (80.5 mL) was added triethyl amine (3.3 g, 114.6 mmol) and methansulfonyl chloride (3.69 g, 32.2 mmol). The resulting mixture was stirred at room temperature for 2 hours. The reaction mixture was concentrated to dryness. The crude residue was purified by column chromatography (SiO₂, EtOAc/hexanes, 0 to 30%) to give the desired product.

Step c: 1,2,3-triazole (117.0 mg, 1.51 mmol) was dissolved in anhydride NMP (2.8 mL) and cooled to 0° C. NaH (60.5 mg, 1.51 mmol, 60% in mineral oil) was added to the mixture and stirred at 0° C. for 1 h. A solution of the product from step b in NMP (5.6 mL) was added to the mixture. The reaction mixture was stirred and heated at 90° C. overnight. After the reaction was complete as judged by NMR, the reaction was cooled to room temperature and poured into water and extracted with EtOAc. The combined organic layers were washed with brine and dried over MgSO₄. Concentration under reduced pressure and purification by reversed phase flash chromatography (SiO₂, 0-100% water/acetonitrile containing 1% formic acid) separated and afforded both regioisomers.

Step d: The desired compounds were prepared in a similar fashion to that described in step f for Example 75.

Step e: The title compounds were prepared in a similar fashion to that described in step g for Example 75.

Example 122: ¹H NMR (400 MHz, CDCl₃) δ 9.99 (s, 1H), 7.55 (s, 2H), 7.40 (t, J=7.8 Hz, 1H), 7.30 (ddd, J=7.9, 2.1, 1.1 Hz, 2H), 7.00 (ddd, J=7.8, 1.9, 1.1 Hz, 1H), 6.89 (t, J=1.9 Hz, 1H), 6.54 (d, J=1.2 Hz, 1H), 6.38 (s, 1H), 4.76 (s, 2H), 3.60 (d, J=2.0 Hz, 2H), 3.40-3.25 (m, 2H), 3.03 (td, J=15.3, 12.7 Hz, 2H), 2.78 (t, J=12.7 Hz, 2H), 1.97-1.82 (m, 2H), 1.76-1.48 (m, 4H), 0.95-0.79 (m, 6H), 0.68-0.59 (m, 2H). ¹⁹F NMR (376 MHz, CDCl₃) δ −84.81 (d, J=198.5 Hz), −91.63 (d, J=198.1 Hz). ESI MS [M+H]⁺ for C₃₀H₃₅F₂N₆O, calcd 533.3, found 533.3.

Example 123: ¹H NMR (400 MHz, CDCl₃) δ 7.48 (s, 1H), 7.40 (t, J=7.7 Hz, 1H), 7.25 (s, 1H), 6.89 (d, J=14.9 Hz, 3H), 6.80 (s, 1H), 6.59 (s, 1H), 5.82 (s, 2H), 4.71 (s, 2H), 4.31 (s, 1H), 4.10 (q, J=7.1 Hz, 1H), 3.41 (s, 1H), 3.32 (s, 1H), 3.21 (dd, J=14.0, 5.4 Hz, 2H), 3.07-2.91 (m, 2H), 2.02 (s, 2H), 1.86 (s, 2H), 1.28-1.19 (m, 3H), 0.92 (t, J=7.5 Hz, 5H), 0.61 (d, J=5.0 Hz, 2H). 1⁹F NMR (376 MHz, CDCl₃) δ −84.41 (d, J=199.6 Hz), −91.40 (d, J=199.4 Hz).

ESI MS [M+H]⁺ for C₃₀H₃₅F₂N₆O, calcd 533.3, found 533.3.

### Example 124: 4-cyclopropyl-6-[3-[3,3-difluoro-1-(triazol-2-ylmethyl)cyclobutyl]phenyl]-2-[[(3S)-3-methylpiperidin-1-yl]methyl]-1H-pyrrolo[2,3-c]pyridin-7-one

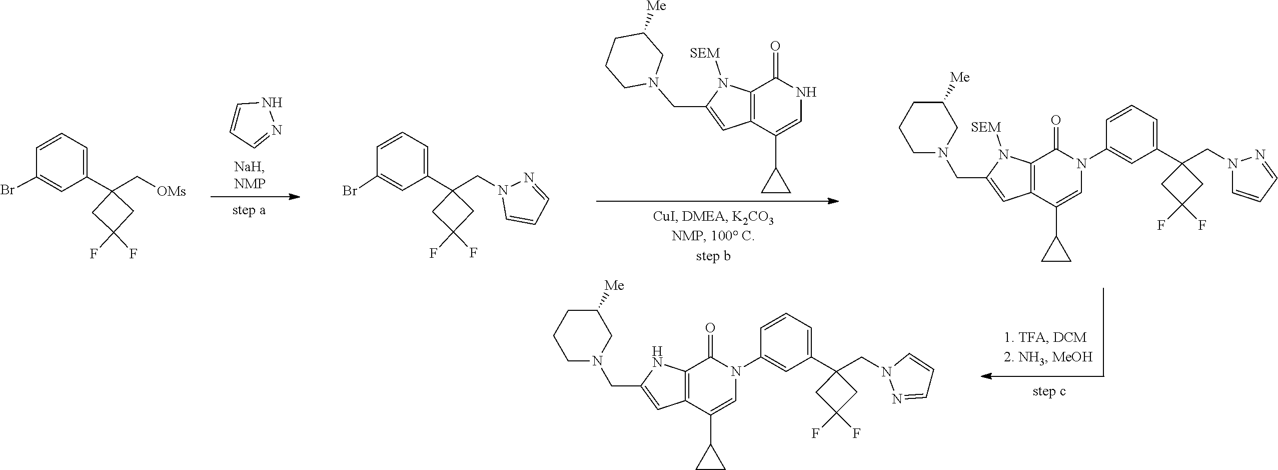 [SMILES: CS(=O)(=O)OCC1(c2cccc(Br)c2)CC(F)(F)C1 ; c1cn[nH]c1 ; FC1(F)CC(Cn2cccn2)(c2cccc(Br)c2)C1 ; C[C@H]1CCCN(Cc2cc3c(C4CC4)c[nH]c(=O)c3n2COCC[Si](C)(C)C)C1 ; C[C@H]1CCCN(Cc2cc3c(C4CC4)cn(-c4cccc(C5(Cn6cccn6)CC(F)(F)C5)c4)c(=O)c3n2COCC[Si](C)(C)C)C1 ; C[C@H]1CCCN(Cc2cc3c(C4CC4)cn(-c4cccc(C5(Cn6cccn6)CC(F)(F)C5)c4)c(=O)c3[nH]2)C1]

Step a: The desired compound was prepared in a similar fashion to that described in step c for Example 122.

Step b: The desired compound was prepared in a similar fashion to that described in step f for Example 75.

Step c: The title compound was prepared in a similar fashion to that described in step g for Example 75. ¹H NMR (400 MHz, CDCl₃) δ 10.27 (s, 1H), 7.49 (s, 1H), 7.38 (t, J=7.8 Hz, 1H), 7.31 (d, J=7.9 Hz, 1H), 6.93-6.84 (m, 2H), 6.74 (d, J=2.3 Hz, 1H), 6.55 (s, 1H), 6.37 (s, 1H), 6.08 (s, 1H), 4.45 (s, 2H), 3.64-3.55 (m, 2H), 3.25 (td, J=14.2, 6.0 Hz, 2H), 3.01 (d, J=14.0 Hz, 1H), 2.94 (d, J=14.3 Hz, 1H), 2.74 (t, J=12.7 Hz, 2H), 1.88 (dq, J=9.8, 5.0 Hz, 2H), 1.73-1.49 (m, 6H), 1.26 (s, 1H), 0.84 (dd, J=33.4, 7.0 Hz, 7H), 0.65 (t, J=5.2 Hz, 2H). ¹⁹F NMR (376 MHz, CDCl₃) δ −84.43 (d, J=197.8 Hz), −91.08 (d, J=197.9 Hz). ESI MS [M+H]⁺ for C₃₁H₃₆F₂N5O, calcd 532.3, found 532.3.

### Example 125: 4-cyclopropyl-6-[3-[1-(4,5-dimethyl-1,2,4-triazol-3-yl)-3,3-difluorocyclobutyl]phenyl]-2-[[(3S)-3-methylpiperidin-1-yl]methyl]-1H-pyrrolo[2,3-c]pyridin-7-one

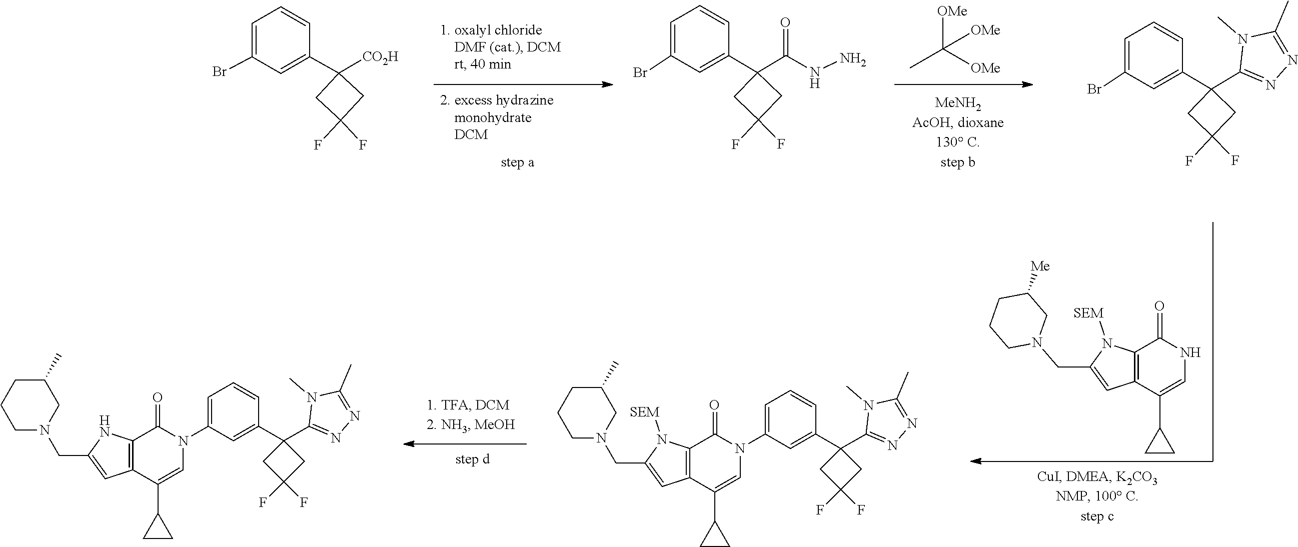

Step a: To a solution of 1-(3-bromophenyl)-3,3-difluorocyclobutane-1-carboxylic acid (1.0 g, 3.43 mmol) in oxalylchloride (2.1 g, 17.18 mmol) was added 1 drop of DMF under nitrogen. The reaction mixture was stirred at room temperature for 40 min. The volatiles were evaporated under reduced pressure. The dried residue was diluted with anhydrous DCM (6.8 mL) and hydrazine hydrate (859.0 mg, 17.18 mmol) was added slowly at 0° C. The resulting mixture was allowed to stir at room temperature overnight. The reaction mixture was partitioned between EtOAc and water, the aqueous phase was extracted three times with EtOAc. The combined organic layers were washed with saturated NaHCO₃ (aq) and brine, and dried over MgSO₄. Concentration under reduced pressure and purification by reversed phase flash chromatography (SiO₂, 0-100% water/acetonitrile containing 1% formic acid) furnished the desired compound.

Step b: To a solution of the product from step a (276.7 mg, 0.907 mmol) in dioxane (2 mL) was added 1,1,1-trimethoxyethane (163.4 mg, 1.36 mmol) and stirred for 1 hour at 45° C. To the above solution was added methylamine hydrochloric acid (91.83 mg, 1.36 mmol) and AcOH (0.1 mL) and stirred at 130° C. overnight. After cooling the solution to room temperature, the reaction mixture was partitioned between EtOAc and water, the aqueous phase was extracted three times with EtOAc. The combined organic layers were dried over MgSO₄. Concentration under reduced pressure and purification by reversed phase flash chromatography (SiO₂, 0-100% water/acetonitrile containing 1% formic acid) furnished the desired compound.

Step c: The desired compound was prepared in a similar fashion to that described in step f for Example 75.

Step d: The title compound was prepared in a similar fashion to that described in step g for Example 75. ¹H NMR (400 MHz, CDCl₃) δ 9.85 (s, 1H), 7.48 (t, J=7.9 Hz, 1H), 7.39-7.28 (m, 3H), 6.72 (d, J=1.2 Hz, 1H), 6.39 (s, 1H), 3.73 (tdd, J=12.6, 10.8, 2.7 Hz, 2H), 3.61 (d, J=1.6 Hz, 2H), 3.33 (tdd, J=13.5, 9.2, 2.7 Hz, 2H), 3.22 (s, 3H), 2.85-2.74 (m, 2H), 2.38 (s, 3H), 2.00-1.82 (m, 2H), 1.75-1.50 (m, 5H), 0.96-0.80 (m, 7H), 0.71-0.60 (m, 2H). ¹⁹F NMR (376 MHz, CDCl₃) δ −87.30 (d, J=196.9 Hz), −88.68 (d, J=196.9 Hz). ESI MS [M+H]⁺ for C₃₁H₃₇F₂N₆O, calcd 547.3, found 547.3

### Example 126: 4-cyclopropyl-6-(3-((1r,3S)-3-fluoro-1-(1-methyl-1H-imidazol-2-yl)cyclobutyl)phenyl)-2-(((S)-3-methylpiperidin-1-yl)methyl)-1,6-dihydro-7H-pyrrolo[2,3-c]pyridin-7-one

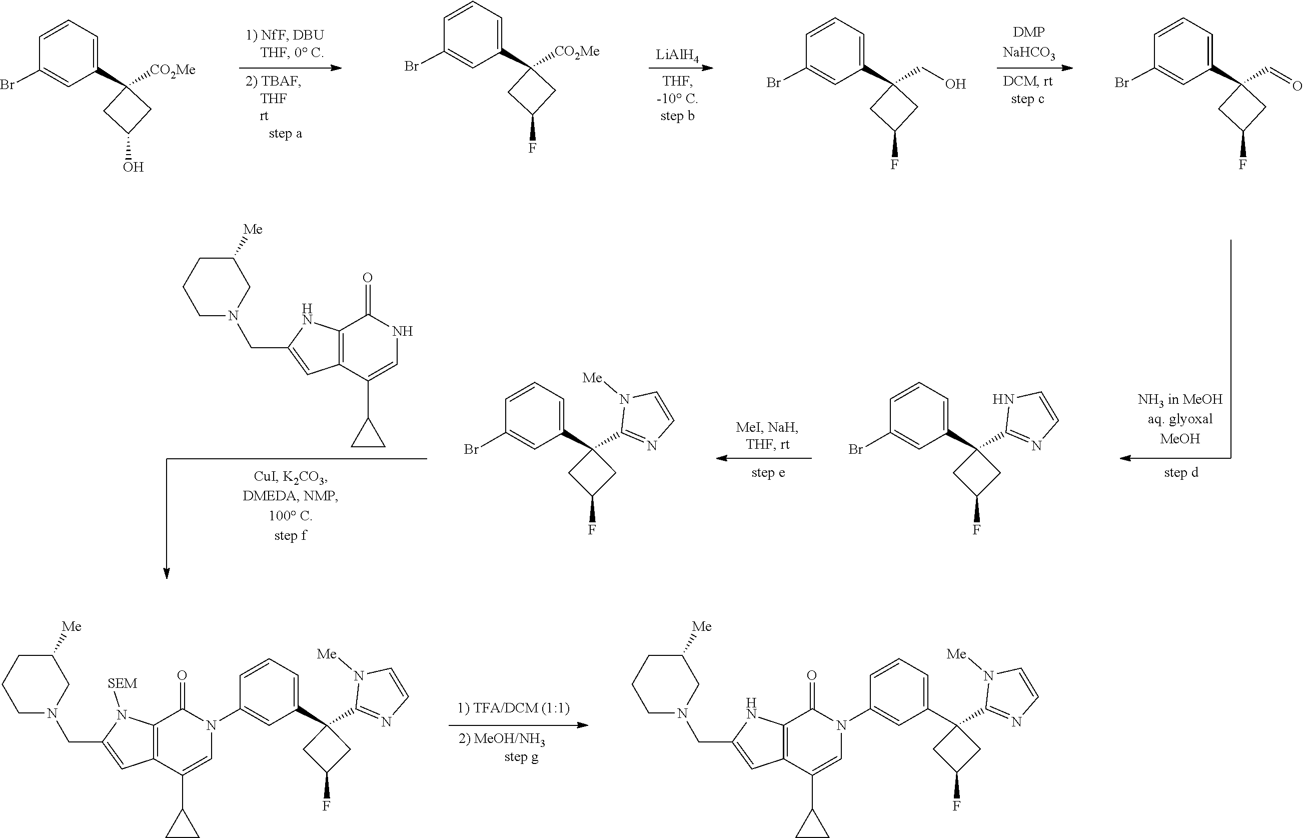

Step a: To a solution of methyl (1s,3s)-1-(3-bromophenyl)-3-hydroxycyclobutane-1-carboxylate (5.68 g, 20 mmol, 1.0 equiv.) in THF (200 mL, 0.1 M), Nonafluorobutanesulfonyl fluoride (NfF) (9.06 g, 30 mmol, 1.5 equiv.) was added at 0° C. After 5 minutes, DBU (3.34 g, 21 mmol, 1.1 equiv.) was added to the reaction mixture and stirred for 12 h at rt. Then, the reaction mixture was quenched with sat. aq. NaHCO₃ and extracted with DCM. The combined organic phase was dried over Na₂SO₄, and concentrated to dryness. The crude residue was dissolved in THF (200 mL, 0.2 M) and TBAF was added to the reaction mixture. After 1 h, the reaction mixture was quenched with sat. aq. NH₄Cl and extracted with DCM. The combined organic phase was dried over Na₂SO₄, concentrated and the crude residue was purified by column chromatography (SiO₂, EtOAc in Hexane, 0 to 40%) to give methyl (1r,3r)-1-(3-bromophenyl)-3-fluorocyclobutane-1-carboxylate as the desired product.

Step b: To a solution of a methyl (1r,3r)-1-(3-bromophenyl)-3-fluorocyclobutane-1-carboxylate (3.2 g, 11.3 mmol, 1.0 equiv.) in THE (55 mL, 0.2 M), lithium aluminum hydride (11.3 mL, 22.6 mmol, 2.0 equiv.) was added at −10° C. and the mixture was stirred for 0.5 h. At the same temperature, the reaction mixture was diluted with diethyl ether and slowly quenched by adding 0.8 mL of water. Then, 0.8 mL of 15% NaOH and 2.4 mL of H₂O were added to the reaction mixture and stirred at 0° C. for 15 min. The suspension was dried over MgSO₄, filtered, concentrated and the crude residue purified by column chromatography (SiO₂, ethyl acetate in hexane, 0 to 50%) to give ((1r,3r)-1-(3-bromophenyl)-3-fluorocyclobutyl)methanol.

Step c: To the product of step b (2.9 g, 11.24 mmol, 1.0 equiv.) in DCM (56 mL, 0.2 M) was added NaHCO₃ (1.88 g, 22.48 mmol, 2.0 equiv.) and DMP (5.71 g, 13.48 mmol, 1.2 equiv.) at 0° C. The resulting mixture was stirred at rt for 1 h. The reaction was quenched with sat. aq. Na₂S₂O₃ solution, the organic phase was separated, and the aqueous layer was extracted with DCM. The combined organic phase was dried over Na₂SO₄, concentrated, and the crude residue was purified by column chromatography (SiO₂, EtOAc in hexane, 0 to 40%) to afford (1r,3r)-1-(3-bromophenyl)-3-fluorocyclobutane-1-carbaldehyde.

Step d: To a solution of the product from step c (2.9 g, 11.32 mmol, 1.0 equiv.) in MeOH (56 mL, 0.2 M) was added aq. glyoxal (9.04 mL, 79.2 mmol, 7.0 equiv.) and NH₃ in MeOH (16.17 mL, 113.2 mmol, 7.0 equiv.). The resulting mixture was stirred at rt for 48 h. After 48 h, another portion of aq. glyoxal (9.04 mL, 79.2 mmol, 7.0 equiv.) and NH₃ in MeOH (16.17 mL, 113.2 mmol, 7.0 equiv.) were added. After 48 h, the reaction mixture was concentrated to dryness, dissolved in EtOAc, and washed with water and brine. The organic phase was dried over Na₂SO₄, concentrated and the crude residue was purified by column chromatography (SiO₂, EtOAc in hexane, 0 to 60%) to give the product 2-((1r,3r)-1-(3-bromophenyl)-3-fluorocyclobutyl)-1H-imidazole.

Step e: To a solution of the product from step d (250 mg, 0.84 mmol, 1.0 equiv.) in THF (4.2 mL, 0.2 M) was added NaH (170 mg, 60% in mineral oil, 4.2 mmol, 5.0 equiv.) at 0° C. After 20 minutes, MeI (0.84 g, 5.92 mmol, 7.0 equiv.) was added to the reaction mixture and stirred for 1.5 h. On completion, the reaction mixture was quenched with water, and the aqueous layer was extracted with EtOAc. The combined organic phase was dried over Na₂SO₄, concentrated and the crude residue was purified by column chromatography (SiO₂, EtOAc in hexanes, 10 to 70%) to give 2-((1r,3r)-1-(3-bromophenyl)-3-fluorocyclobutyl)-1-methyl-1H-imidazole.

Step f: The reaction was performed in a similar fashion to Example 18, step d.

Step g: The desired product was prepared in a similar manner to Example 7, step e. ¹H NMR (400 MHz, Methanol-d4) δ 8.46 (s, 1H), 7.51 (t, J=7.9 Hz, 1H), 7.29-7.21 (m, 2H), 7.21-7.17 (m, 1H), 7.02 (d, J=1.4 Hz, 1H), 6.91 (d, J=1.4 Hz, 1H), 6.81 (d, J=1.1 Hz, 1H), 6.69 (s, 1H), 5.13-4.89 (m, 1H), 4.15-4.04 (m, 2H), 3.46-3.34 (m, 2H), 3.31 (s, 3H), 3.25-3.08 (m, 2H), 2.95-2.78 (m, 2H), 2.53-2.40 (m, 1H), 2.24-2.10 (m, 1H), 2.00-1.87 (m, 1H), 1.87-1.62 (m, 4H), 1.10-0.97 (m, 1H), 0.93 (d, J=6.4 Hz, 3H), 0.92-0.86 (m, 2H), 0.69-0.61 (m, 2H).

### Example 127: 4-cyclopropyl-6-[3-[3,3-difluoro-1-(1,3-oxazol-2-yl)cyclobutyl]phenyl]-2-[[(3S)-3-methylpiperidin-1-yl]methyl]-1H-pyrrolo[2,3-c]pyridin-7-one

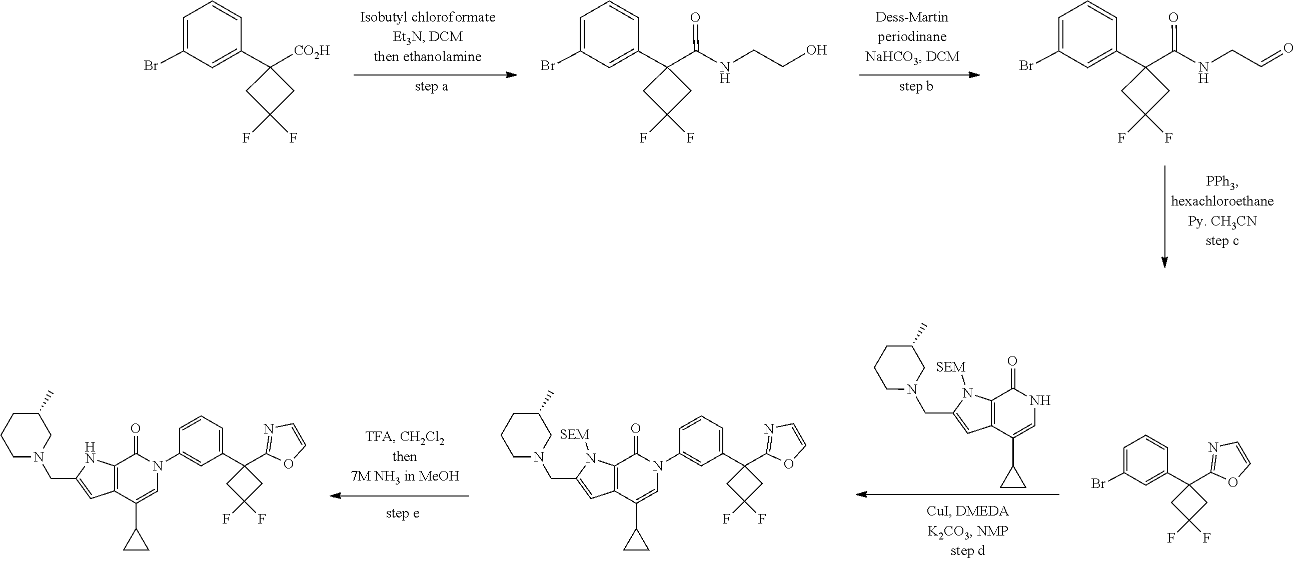

Step a: Triethylamine (0.6 mL, 4.3 mmol) was added to a solution 1-(3-bromophenyl)-3,3-difluorocyclobutane-1-carboxylic acid (0.5 g, 1.7 mmol) in dichloromethane (8.6 mL). The resulting solution was placed in 40 mL screw cap vial equipped with magnetic stirring bar and nitrogen balloon attached through a rubber septum. The reaction was cooled to 0° C. before isobutyl chloroformate (0.25 mL, 1.9 mmol) was added dropwise over 1 min. The reaction mixture was allowed to warm and stirred for 1 h at 23° C. The brownish solution was cooled back to 0° C. and ethanolamine (0.41 mL, 6.7 mmol) was added dropwise over 2 min. The reaction mixture was stirred at 23° C. for 30 min, diluted with dichloromethane (20.0 mL) and poured into aq. sat. sodium bicarbonate solution (20.0 mL). The organic phase was separated, and the aqueous phase was additionally extracted with dichloromethane (2×15.0 mL). The combined organic extract was dried over sodium sulfate, and all volatiles were removed under reduced pressure. The crude product obtained upon concentration was used directly for step b.

Step b: The crude product from step a (1.7 mmol) was dissolved in dichloromethane (8.6 mL), and the solution was placed in 40 mL screw cap vial equipped with magnetic stirring bar. The reaction mixture was cooled to 0° C., and sodium bicarbonate (0.70 g, 8.6 mmol) was added followed by Dess-Martin periodinane (0.90 g, 2.1 mmol). The cooling bath was removed, and the reaction mixture was stirred at 23° C. for 1 h. The resulting clear solution was diluted with dichloromethane (10.0 mL) and aq. sat. Na₂S₂O₃ (10.0 mL). The obtained biphasic mixture was vigorously stirred for 30 min. The organic phase was separated, and the aqueous phase was additionally extracted with dichloromethane (2×15.0 mL). The combined organic extract was dried over sodium sulfate, filtered, and concentrated to dryness. The crude product was purified by column chromatography (SiO₂, hexanes/EtOAc gradient) to produce the desired aldehyde.

Step c: Hexachloroethane (0.43 g, 1.8 mmol) was added to a solution of triphenylphosphine (0.47 g, 1.8 mmol) in acetonitrile (8.0 mL). The aldehyde obtained in step b (0.30 g, 0.9 mmol) was added directly to the aforementioned mixture as a solution in acetonitrile (4.0 mL). The reaction mixture was stirred at 0° C. for 15 min. Once precipitate formation was observed, pyridine (0.30 mL, 3.6 mmol) was added, and the resulting reaction mixture was left to stir at room temperature overnight. The next day the reaction was diluted with ethyl acetate (20.0 mL) and brine (10.0 mL). The organic phase was separated, and the aqueous phase was additionally extracted with ethyl acetate (2×15.0 mL). The combined organic extract was dried over sodium sulfate, filtered, and concentrated to dryness. The crude product was purified by column chromatography (SiO₂, hexanes/EtOAc gradient) to produce the desired oxazole-containing product.

Step d: The bromide obtained in step c (68.0 mg, 0.22 mmol) was combined with 4-cyclopropyl-2-[[(3S)-3-methylpiperidin-1-yl]methyl]-1-(2-trimethylsilylethoxymethyl)-6H-pyrrolo[2,3-c]pyridin-7-one (62 mg, 0.22 mmol), 1,2-dimethylethylenediamine (44 uL, 0.44 mmol) and potassium carbonate (91.0 mg, 0.66 mmol) in dry NMP (2.2 mL). This mixture was placed in 40 mL screw cap vial equipped with magnetic stirring bar, degassed under vacuum and backfilled with nitrogen (repeated 3 times). Copper (I) iodide (42.0 mg, 0.22 mmol) was added, the mixture was degassed again using previous method and heated at 100° C. for 2 h. Then the mixture was cooled to ambient temperature, diluted with aq. sat. NH₄Cl (5.0 mL), EtOAc (15.0 mL) and water (10.0 mL). The organic phase was separated, and the aqueous phase was extracted with ethyl acetate (2×15.0 mL). The combined organic extract was washed with water (3×30.0 mL) dried over sodium sulfate, filtered, and concentrated to dryness. The crude product was purified by reversed phase column chromatography (C18-modified SiO₂, water/CH₃CN gradient containing with 0.1% formic acid) to produce the desired coupling product.

Step e: The product of step d (60.0 mg, 0.1 mmol) was dissolved in dichloromethane (0.5 mL). The solution was cooled to 0° C., trifluoroacetic acid (0.5 mL) was added, and the reaction allowed to stir at 23° C. for 1 h. The resulting brownish mixture was concentrated to dryness under reduced pressure, and the dry residue was redissolved in 4 ml of ammonia in MeOH (7 M solution). After 1 h of stirring the solution was concentrated and directly fractionated by reversed phase column chromatography (C18-modified SiO₂, water/CH₃CN gradient containing with 0.1% formic acid) to produce the title product. ¹H NMR (400 MHz, CDCl₃) δ 10.07 (br. s, 1H), 7.58 (d, J=0.8 Hz, 1H), 7.53-7.44 (m, 1H), 7.39 (t, J=1.9 Hz, 1H), 7.35 (d, J=8.3 Hz, 2H), 7.06 (d, J=0.9 Hz, 1H), 6.70 (d, J=1.2 Hz, 1H), 6.38 (s, 1H), 3.75-3.51 (m, 4H), 3.44-3.23 (m, 2H), 2.76 (t, J=11.7 Hz, 2H), 1.95-1.81 (m, 2H), 1.75-1.46 (m, 5H), 0.94-0.75 (m, 6H), 0.78-0.57 (m, 2H). ¹⁹F NMR (376 MHz, CDCl₃) δ −85.39 (d, J=196.4 Hz), −93.11 (d, J=196.4 Hz).

### Example 128: 4-cyclopropyl-6-[3-[3,3-difluoro-1-(1-methylimidazol-2-yl)cyclobutyl]phenyl]-2-[[(3S)-3-methylpiperidin-1-yl]methyl]-1H-pyrrolo[2,3-c]pyridin-7-one

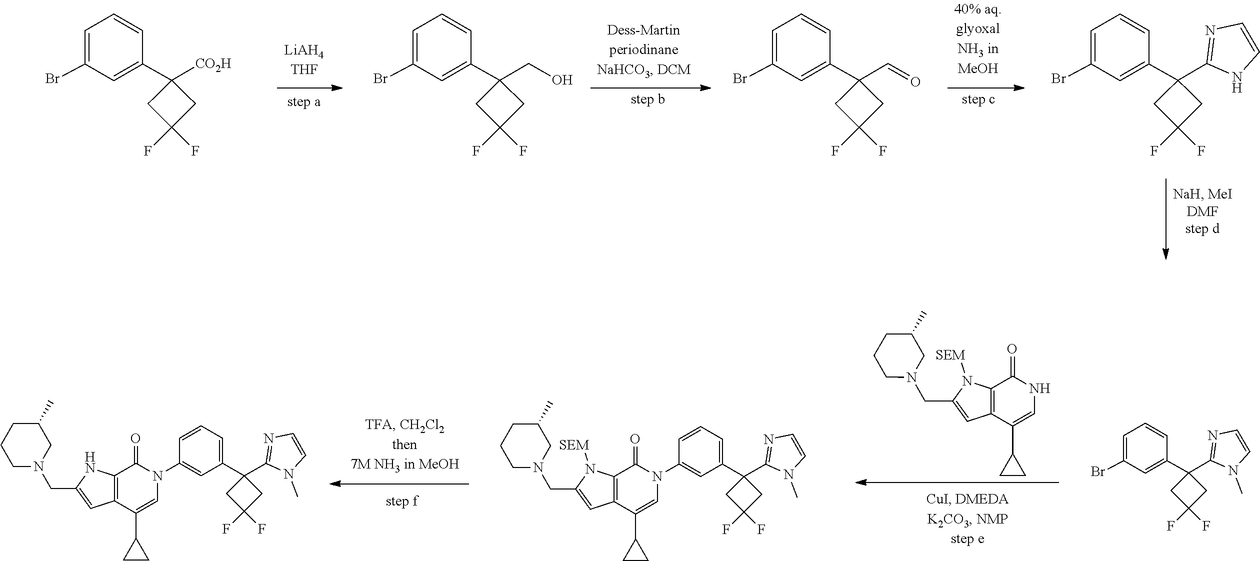

Step a: A solution of 1-(3-bromophenyl)-3,3-difluorocyclobutane-1-carboxylic acid (0.60 g, 2.1 mmol) in dry tetrahydrofuran (10.0 mL) was cooled to 0° C. under nitrogen. Then LiAlH₄ (1.05 mL, 2.1 mmol, 2 M solution in THF) was added dropwise over 10 min. The resulting colorless solution was maintained at 0° C. before it was quenched using the Fieser workup protocol. Upon removal of inorganic precipitate the resulting solution was concentrated under reduced pressure to produce the corresponding alcohol.

Step b: The crude product from step a (2.1 mmol) was dissolved in dichloromethane (9.0 mL), and the solution was placed in 40 mL screw cap vial equipped with magnetic stirring bar. The reaction mixture was cooled to 0° C., and sodium bicarbonate (0.60 g, 7.2 mmol) was added followed by Dess-Martin periodinane (0.92 g, 2.1 mmol). The cooling bath was removed, and the reaction mixture was stirred at 23° C. for 1 h. The resulting clear solution was diluted with dichloromethane (10.0 mL) and aq. sat. Na₂S₂O₃ (10.0 mL). The obtained biphasic mixture was vigorously stirred for 30 min. The organic phase was separated, and the aqueous phase was additionally extracted with dichloromethane (2×15.0 mL). The combined organic extract was dried over sodium sulfate, filtered, and concentrated to dryness. The crude product was purified by column chromatography (SiO₂, hexanes/EtOAc gradient) to produce the desired aldehyde.

Step c: The aldehyde from step b (0.27 g, 1.0 mmol) was combined with aq. glyoxal (0.84 mL, 6.0 mmol, 40 wt % solution), ammonia in MeOH (0.4 mL, 3.0 mmol, 7 M solution) and additional MeOH (2.0 mL) at room temperature. The resulting mixture was placed in 40 mL screw cap vial equipped with magnetic stirring bar and stirred at room temperature for 24 h. The resulting mixture was diluted with water (10 mL) and extracted with EtOAc (3×10 mL). The combined organic extract was dried over sodium sulfate, filtered, and concentrated to dryness. The crude product was purified by column chromatography (SiO₂, hexanes/EtOAc gradient) to produce the desired imidazole.

Step d: A solution of imidazole from step c (50.0 mg, 0.16 mmol) in tetrahydrofuran (1.6 mL) was cooled to 0° C., and sodium hydride (20.0 mg, 0.8 mmol) was added in one portion. The reaction mixture was stirred for 5 min, then MeI (70 uL, 1.1 mmol) was added via syringe. The resulting mixture was allowed to warm and stirred at room temperature for 2 h. The reaction was quenched by careful addition of aq. sat. NH₄Cl (1.0 mL), then diluted with water (10.0 mL) and EtOAc (10.0 mL). The organic phase was separated, and the aqueous phase was additionally extracted with EtOAc (2×5.0 mL). The combined organic extract was dried over sodium sulfate, and all volatiles were removed under reduced pressure. The crude product was purified by column chromatography (SiO₂, hexanes/EtOAc gradient) to produce the desired N-methylimidazole.

Step e: The bromide obtained in step d (40.0 mg, 0.12 mmol) was combined with 4-cyclopropyl-2-[[(3S)-3-methylpiperidin-1-yl]methyl]-1-(2-trimethylsilylethoxymethyl)-6H-pyrrolo[2,3-c]pyridin-7-one (35 mg, 0.12 mmol), 1,2-dimethylethylenediamine (24 uL, 0.24 mmol) and potassium carbonate (51.0 mg, 0.37 mmol) in dry NMP (1.2 mL). This mixture was placed in 40 mL screw cap vial equipped with magnetic stirring bar, degassed under vacuum and backfilled with nitrogen (repeated 3 times). Copper (I) iodide (23.0 mg, 0.12 mmol) was added, the mixture was degassed again using the previous method and heated at 100° C. for 1 h. Then the mixture was cooled to ambient temperature, and diluted with aq. sat. NH₄Cl (5.0 mL), EtOAc (15.0 mL) and water (10.0 mL). The organic phase was separated, and the aqueous phase was extracted with ethyl acetate (2×15.0 mL). The combined organic extract was washed with water (3×10.0 mL) dried over sodium sulfate, filtered, and concentrated to dryness. The crude product was purified by reversed phase column chromatography (C18-modified SiO₂, water/CH₃CN gradient containing with 0.1% formic acid) to produce the desired coupling product.

Step f: The product of step e (32.0 mg, 0.05 mmol) was dissolved in dichloromethane (0.5 mL). The solution was cooled to 0° C., trifluoroacetic acid (0.5 mL) was added, and the reaction allowed to stir at 23° C. for 1 h. The resulting brownish mixture was concentrated to dryness under reduced pressure, and the dry residue was redissolved in 4 ml of ammonia in MeOH (7 M solution). After 1 h of stirring the solution was concentrated and directly fractionated by reversed phase column chromatography (C18-modified SiO₂, water/CH₃CN gradient containing with 0.1% formic acid) to produce the title product. ¹H NMR (400 MHz, CDCl₃) δ 9.81 (br. s, 1H), 7.44 (t, J=8.2 Hz, 1H), 7.31-7.18 (m, 3H), 6.98 (d, J=1.2 Hz, 1H), 6.83 (d, J=1.4 Hz, 1H), 6.70 (d, J=1.2 Hz, 1H), 6.39 (s, 1H), 3.84-3.43 (m, 4H), 3.36-3.12 (m, 5H), 2.03-1.91 (m, 1H), 1.92-1.79 (m, 1H), 1.76-1.54 (m, 5H), 1.00-0.74 (m, 6H), 0.69-0.54 (m, 2H). ¹⁹F NMR (376 MHz, CDCl₃) δ −87.23 (d, J=196.2 Hz), −89.04 (d, J=196.3 Hz).

### Example 129: cis-4-cyclopropyl-6-[3-[3-fluoro-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

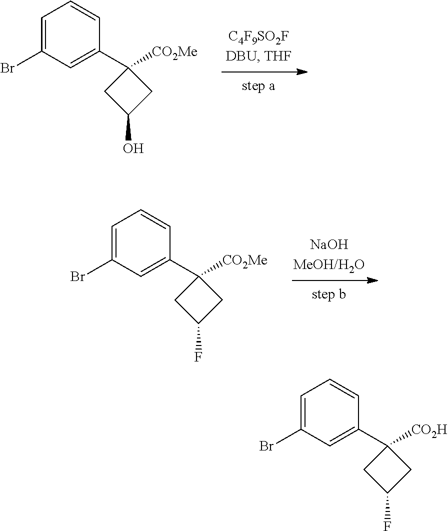 [SMILES: COC(=O)[C@]1(c2cccc(Br)c2)C[C@@H](O)C1 ; COC(=O)[C@]1(c2cccc(Br)c2)C[C@H](F)C1 ; O=C(O)[C@]1(c2cccc(Br)c2)C[C@H](F)C1]

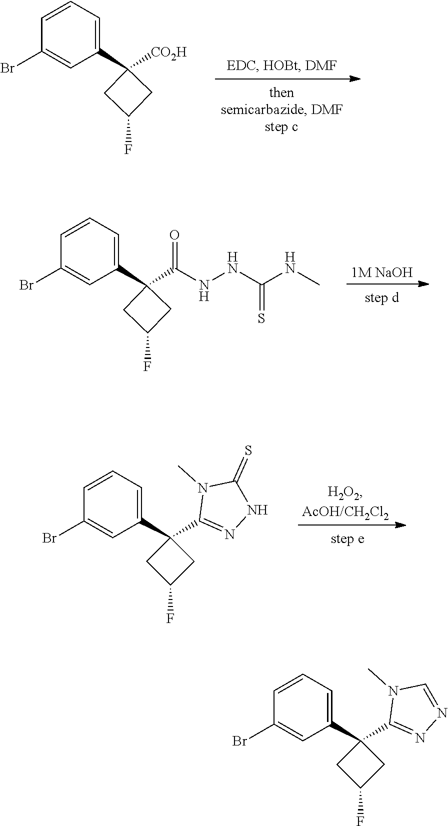

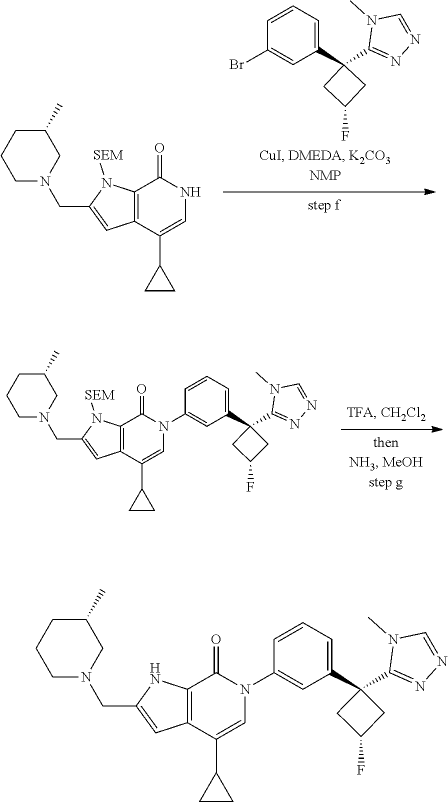 [SMILES: C[C@H]1CCCN(Cc2cc3c(C4CC4)c[nH]c(=O)c3n2COCC[Si](C)(C)C)C1 ; Cn1cnnc1[C@]1(c2cccc(Br)c2)C[C@H](F)C1 ; C[C@H]1CCCN(Cc2cc3c(C4CC4)cn(-c4cccc([C@]5(c6nncn6C)C[C@@H](F)C5)c4)c(=O)c3n2COCC[Si](C)(C)C)C1 ; C[C@H]1CCCN(Cc2cc3c(C4CC4)cn(-c4cccc([C@]5(c6nncn6C)C[C@@H](F)C5)c4)c(=O)c3[nH]2)C1]

Step a: To a solution of trans-methyl 1-(3-bromophenyl)-3-hydroxycyclobutane-1-carboxylate (5.68 g, 20 mmol) in THF (200 mL, 0.1 M), nonafluorobutanesulfonyl fluoride (NfF) (9.06 g, 30 mmol) was added at 0° C. After 5 minutes, DBU (3.34 g, 21 mmol) was added to the reaction mixture and stirred for 12 h at rt. Then, the reaction mixture was quenched with sat. aq. NaHCO₃ and extracted with DCM. The combined organic phase was dried over Na₂SO₄, concentrated to dryness. The crude residue was dissolved in THF (200 mL, 0.2 M) and TBAF was added to the reaction mixture. After 1 h, the reaction mixture was quenched with sat. aq. NH₄Cl and extracted with DCM. The combined organic phase was dried over Na₂SO₄, concentrated and the crude residue was purified by column chromatography (SiO₂, EtOAc in Hexane, 0 to 40%) to give the desired γ-fluoroester.

Step b: A mixture of γ-fluoroester from step a (0.8 g, 2.8 mmol) and sodium hydroxide (0.56 g, 14.1 mmol) in MeOH (7.0 mL) and water (7.0 mL) was maintained at 70° C. for 1 h. The reaction was cooled to room temperature, acidified with 1 M aq. HCl to pH˜1, and the product was extracted with ethyl acetate (3×15.0 mL). The combined organic extract was dried over sodium sulfate and concentrated to dryness to yield the desired γ-fluoroacid.

Step c: The product from step b (1.35 g) was dissolved in DMF (21 mL), and EDC (1.20 g, 6.30 mmol) followed by HOBt (0.96 g, 6.30 mmol) were added. The reaction was stirred for 1 h at 23° C. The mixture was next diluted with EtOAc (20 mL) and washed with water (3×20 mL). The organic layer was dried over Na₂SO₄ and evaporated to dryness. The dry residue was redissolved in DMF (21 mL), and 4-methyl-3-thiosemicarbazide (0.53 g, 5.0 mmol) was added. The reaction was stirred for 15 h at 50° C. After completion, the reaction mixture was cooled to rt and dissolved with EtOAc (20 mL). Organic phase was washed with water (3×15 mL), dried over Na₂SO₄, and concentrated to dryness. The dry residue was used in the next step without further purification.

Step d: The product from step c was treated with aq. 1 M NaOH solution (21 mL) for 30 min at 65° C. The reaction mixture was cooled down, acidified to pH=1 with 1 M HCl, and extracted with EtOAc (2×15 mL). The combined organic phase was washed with brine, dried over Na₂SO₄, concentrated under reduced pressure. The crude residue was used in the next step without further purification.

Step e: Acetic acid (2.20 mL) and hydrogen peroxide (1.10 mL) were added to a solution of the product from step d (1.33 g) in CH₂Cl₂ (17 mL) at 0° C. The reaction was warmed up to room temperature and stirred for 16 h. The mixture was carefully basified to pH=14 with aq. 1 M NaOH and extracted with CH₂Cl₂ (2×10 mL). The combined organic phase was dried over Na₂SO₄ and concentrated to dryness. The crude material was purified by flash chromatography (SiO₂, EtOAc in Hex 0 to 60%, then MeOH in CH₂Cl₂ 0 to 10%) to give triazole thiol as white foam.

Step f: A solution of amide (0.15 g, 0.36 mmol), triazole from step e (0.13 g, 0.36 mmol) and K₂CO₃ (0.15 g, 1.10 mmol) in NMP (3.60 ml) was degassed with a stream of bubbling nitrogen for ten minutes. CuI (69 mg, 0.36 mmol) and DMEDA (71 μL, 0.72 mmol) were added, and the reaction was heated for 2 h at 100° C. The reaction mixture was cooled to rt, quenched with aq. NH₃Cl and extracted with EtOAc (2×7 mL). The combined organic phase was washed with water (2×5 mL), then brine, dried over Na₂SO₄, and concentrated to dryness. The crude material was purified by flash chromatography (SiO₂, EtOAc in Hex 0 to 100%) to give pyrrolopyridone as white foam.

Step g: Pyrrolopyridone from step f (95 mg, 0.15 mmol) was dissolved in CH₂Cl₂ (0.75 mL) and TFA (0.75 mL) was added. The mixture was stirred for 1 h and then concentrated. To this crude material was added 7 M NH₃ in methanol (3.0 mL) and the mixture was stirred for 30 min. The reaction mixture was concentrated, and the crude product was purified by prep-HPLC (20% to 90% MeCN/water, 0.1% TFA) to afford the title compound. ¹H NMR (400 MHz, CDCl₃) δ 10.04 (s, 1H), 7.99 (s, 1H), 7.46 (t, J=7.9 Hz, 1H), 7.40 (t, J=2.0 Hz, 1H), 7.31-7.26 (m, 2H), 6.70 (d, J=1.3 Hz, 1H), 6.39 (s, 1H), 5.42-5.10 (m, 1H), 3.62 (s, 2H), 3.39-3.14 (m, 7H), 2.79 (dd, J=16.6, 9.4 Hz, 2H), 2.00-1.80 (m, 2H), 1.63 (dddd, J=22.8, 19.4, 15.3, 7.5 Hz, 5H), 0.92-0.75 (m, 6H), 0.67-0.55 (m, 2H). ESI MS [M+H]⁺ for C₃₀H₃₅FN₆O, calcd 515.3, found 515.3.

### Example 130: trans-4-cyclopropyl-6-[3-[3-fluoro-1-(4-methyl-1,2,4-triazol-3-yl)cyclobutyl]phenyl]-2-[[(3S)-3-methylpiperidin-1-yl]methyl]-1H-pyrrolo[2,3-c]pyridin-7-one

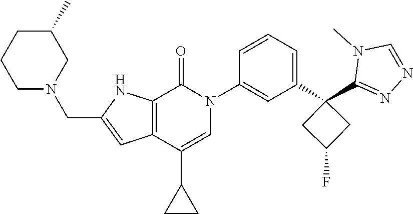 [SMILES: C[C@H]1CCCN(Cc2cc3c(C4CC4)cn(-c4cccc([C@]5(c6nncn6C)C[C@H](F)C5)c4)c(=O)c3[nH]2)C1]

The title compound was prepared in a similar fashion to Example 129 using cis-methyl 1-(3-bromophenyl)-3-fluorocyclobutane-1-carboxylate in step a. ¹H NMR (400 MHz, CDCl₃) δ 9.63 (s, 1H), 8.03 (s, 1H), 7.46 (t, J=7.9 Hz, 1H), 7.39-7.19 (m, 3H), 6.71 (d, J=1.3 Hz, 1H), 6.40 (s, 1H), 5.18 (dq, J=55.7, 6.8 Hz, 1H), 3.70 (s, 2H), 3.55-3.41 (m, 2H), 3.31 (s, 3H), 2.91 (dddd, J=22.1, 10.2, 6.8, 2.8 Hz, 3H), 2.04 (s, 2H), 1.87 (dddd, J=9.6, 8.3, 4.2, 1.2 Hz, 2H), 1.81-1.53 (m, 4H), 0.97-0.78 (m, 6H), 0.70-0.59 (m, 2H). ESI MS [M+H]⁺ for C₃₀H₃₅FN₆O, calcd 515.3, found 515.3.

### Example 131: 4-Cyclopropyl-3-(1-methyl-1H-pyrazol-4-yl)-6-{3-[(1R,3S)-3-methyl-1-(4-methyl-4H-1,2,4-triazol-3-yl)cyclobutyl]phenyl}-1H,6H,7H-pyrrolo[2,3-c]pyridin-7-one

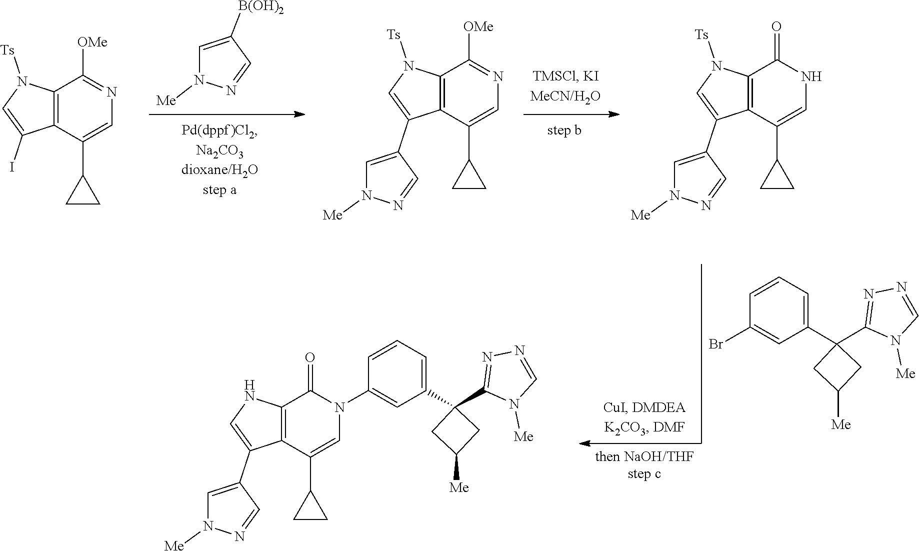 [SMILES: COc1ncc(C2CC2)c2c(I)cn(S(=O)(=O)c3ccc(C)cc3)c12 ; Cn1cc(B(O)O)cn1 ; COc1ncc(C2CC2)c2c(-c3cnn(C)c3)cn(S(=O)(=O)c3ccc(C)cc3)c12 ; Cc1ccc(S(=O)(=O)n2cc(-c3cnn(C)c3)c3c(C4CC4)c[nH]c(=O)c32)cc1 ; Cn1cc(-c2c[nH]c3c(=O)n(-c4cccc([C@]5(c6nncn6C)C[C@@H](C)C5)c4)cc(C4CC4)c23)cn1 ; CC1CC(c2cccc(Br)c2)(c2nncn2C)C1]

Step a: To a 40-mL vial was added 4-cyclopropyl-3-iodo-7-methoxy-1-(4-methylphenyl)sulfonylpyrrolo[2,3-c]pyridine (234 mg, 0.50 mmol, 1.0 equiv.), (1-methylpyrazol-4-yl)boronic acid (75.6 mg, 0.60 mmol, 1.2 equiv.), Pd(dppf)Cl₂ (36.6 mg, 0.050 mmol, 10 mol %), Na₂CO₃ (1 M in H₂O, 1.0 mL, 1.0 mmol, 2.0 equiv.) and dioxane (2.5 mL). The resulting mixture was heated under N₂ at 100° C. for 1.5 h. After cooling to room temperature, the organic phase was separated, and the aqueous layer was extracted with EtOAc. The combined organic phase was dried over Na₂SO₄, concentrated and the crude residue was purified by column chromatography (SiO₂, EtOAc in hexanes, 0 to 50%) to give the product.

Step b: To a mixture of the product from step a (137 mg, 0.32 mmol, 1.0 equiv.) in MeCN/H₂O (4:1 v/v, 2 mL) was added TMSCl (70.0 mg, 0.65 mmol, 2.0 equiv.) and KI (108 mg, 0.65 mmol, 2.0 equiv.). The resulting mixture was heated at 80° C. for 3 h when LCMS showed a completion of the demethylation. After cooling to room temperature, the reaction mixture was concentrated to dryness, and the crude product was purified by column chromatography (SiO₂, MeOH in DCM, 0 to 10%) to give the product.

Step c: To a solution of the product from step b (40.8 mg, 0.10 mmol, 1.0 equiv.), and 3-[1-(3-bromophenyl)-3-methylcyclobutyl]-4-methyl-1,2,4-triazole (diastereomeric mixture in 3:1 ratio, 45.9 mg, 0.15 mmol, 1.5 equiv.) in DMF (1.5 mL) was added CuI (19.0 mg, 0.10 mmol, 1.0 equiv.), N,N′-dimethylethylenediamine (17.6 mg, 0.20 mmol, 2.0 equiv.) and K₂CO₃ (41.5 mg, 0.30 mmol, 3.0 equiv.). The resulting mixture was heated at 110° C. for 3 h. After cooling to room temperature, the reaction mixture was diluted with EtOAc and washed with water twice. The organic phase was then washed with brine, dried over Na₂SO₄ and concentrated. The crude material was then dissolved in THF (2 mL) and treated with 1 M NaOH aqueous solution (2 mL) at 65° C. for overnight. After LCMS showed a full conversion of hydrolysis, the reaction mixture was diluted with H₂O and extracted with EtOAc twice, and then concentrated. The crude residue was purified by HPLC to afford the title compound as a diastereomeric mixture in 3:1 ratio. ¹H NMR (400 MHz, CDCl₃) δ 10.19 (s, 1H), 8.21 (s, 0.24H), 8.15 (s, 0.76H), 7.59 (s, 1H), 7.52-7.35 (m, 4H), 7.33-7.21 (m, 1H), 7.21-7.09 (m, 1H), 6.70 (s, 1H), 3.96 (s, 3H), 3.34 (s, 0.7H), 3.28 (s, 2.3H), 3.22-3.13 (m, 0.5H), 2.89 (q, J=6.6 Hz, 1.5H), 2.73-2.61 (m, 2.3H), 2.37-2.27 (m, 0.7H), 1.76 (ddd, J=14.0, 8.2, 5.7 Hz, 1H), 1.19-1.09 (m, 3H), 0.65-0.54 (m, 2H), 0.55-0.45 (m, 2H). ESI MS [M+H]⁺ for C₂₈H₂₉N₇O, calcd 480.3, found 480.2.

### Example 132: 4-Cyclopropyl-3-(1-methyl-1H-pyrazol-5-yl)-6-{3-[(1R,3S)-3-methyl-1-(4-methyl-4H-1,2,4-triazol-3-yl)cyclobutyl]phenyl}-1H,6H,7H-pyrrolo[2,3-c]pyridin-7-one

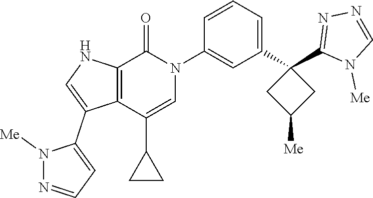 [SMILES: Cn1nccc1-c1c[nH]c2c(=O)n(-c3cccc([C@]4(c5nncn5C)C[C@@H](C)C4)c3)cc(C3CC3)c12]

The title compound in 4:1 diastereomeric ratio was prepared in a similar fashion to that described for Example 131 from 4-cyclopropyl-3-iodo-7-methoxy-1-(4-methylphenyl)sulfonylpyrrolo[2,3-c]pyridine and (2-methylpyrazol-3-yl)boronic acid. 1H NMR (400 MHz, CDCl₃) δ 10.54 (s, 1H), 8.03 (s, 0.23H), 7.97 (s, 0.77H), 7.60-7.38 (m, 4H), 7.36-7.21 (m, 2H), 6.70 (s, 1H), 6.33 (s, 1H), 3.74 (s, 3H), 3.32 (s, 0.6H), 3.27 (s, 2.4H), 3.23-3.13 (m, 0.4H), 3.00-2.80 (m, 1.6H), 2.76-2.58 (m, 2.6H), 2.37-2.26 (m, 0.4H), 1.45 (td, J=7.9, 3.6 Hz, 1H), 1.20-1.07 (m, 3H), 0.54-0.41 (m, 2H), 0.43-0.33 (m, 2H). ESI MS [M+H]⁺ for C₂₈H₂₉N₇O, calcd 480.3, found 480.2.

### Example 133: 4-Cyclopropyl-3-(1-methyl-1H-1,2,3-triazol-5-yl)-6-{3-[(1R,3S)-3-methyl-1-(4-methyl-4H-1,2,4-triazol-3-yl)cyclobutyl]phenyl}-1H,6H,7H-pyrrolo[2,3-c]pyridin-7-one

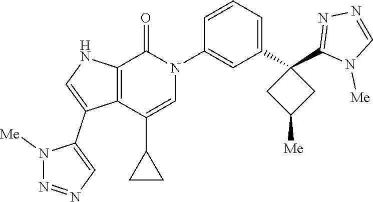 [SMILES: Cn1nncc1-c1c[nH]c2c(=O)n(-c3cccc([C@]4(c5nncn5C)C[C@@H](C)C4)c3)cc(C3CC3)c12]

The title compound in 6:1 diastereomeric ratio was prepared in a similar fashion to that described for Example 131 from 4-cyclopropyl-3-iodo-7-methoxy-1-(4-methylphenyl)sulfonylpyrrolo[2,3-c]pyridine and (3-methyltriazol-4-yl)boronic acid. ¹H NMR (400 MHz, CDCl₃) δ 11.35 (s, 1H), 8.05 (s, 0.16H), 7.98 (s, 0.84H), 7.74 (s, 1H), 7.56-7.43 (m, 2H), 7.41-7.22 (m, 3H), 6.73 (s, 1H), 3.94 (s, 3H), 3.32 (s, 0.4H), 3.27 (s, 2.6H), 3.19 (td, J=8.9, 8.4, 4.4 Hz, 0.3H), 2.96-2.80 (m, 1.7H), 2.77-2.56 (m, 2.7H), 2.32 (t, J=10.0 Hz, 0.3H), 1.36 (td, J=8.2, 4.2 Hz, 1H), 1.14 (d, J=4.7 Hz, 3H), 0.49-0.40 (m, 2H), 0.42-0.33 (m, 2H).

ESI MS [M+H]⁺ for C₂₇H₂₈N₈O, calcd 481.2, found 481.2.

### Example 134: 4-Cyclopropyl-2-{[(3S)-3-methylpiperidin-1-yl]methyl}-7-oxo-6-{3-[(1R,3S)-3-methyl-1-(4-methyl-4H-1,2,4-triazol-3-yl)cyclobutyl]phenyl}-1H,6H,7H-pyrrolo[2,3-c]pyridine-3-carbonitrile

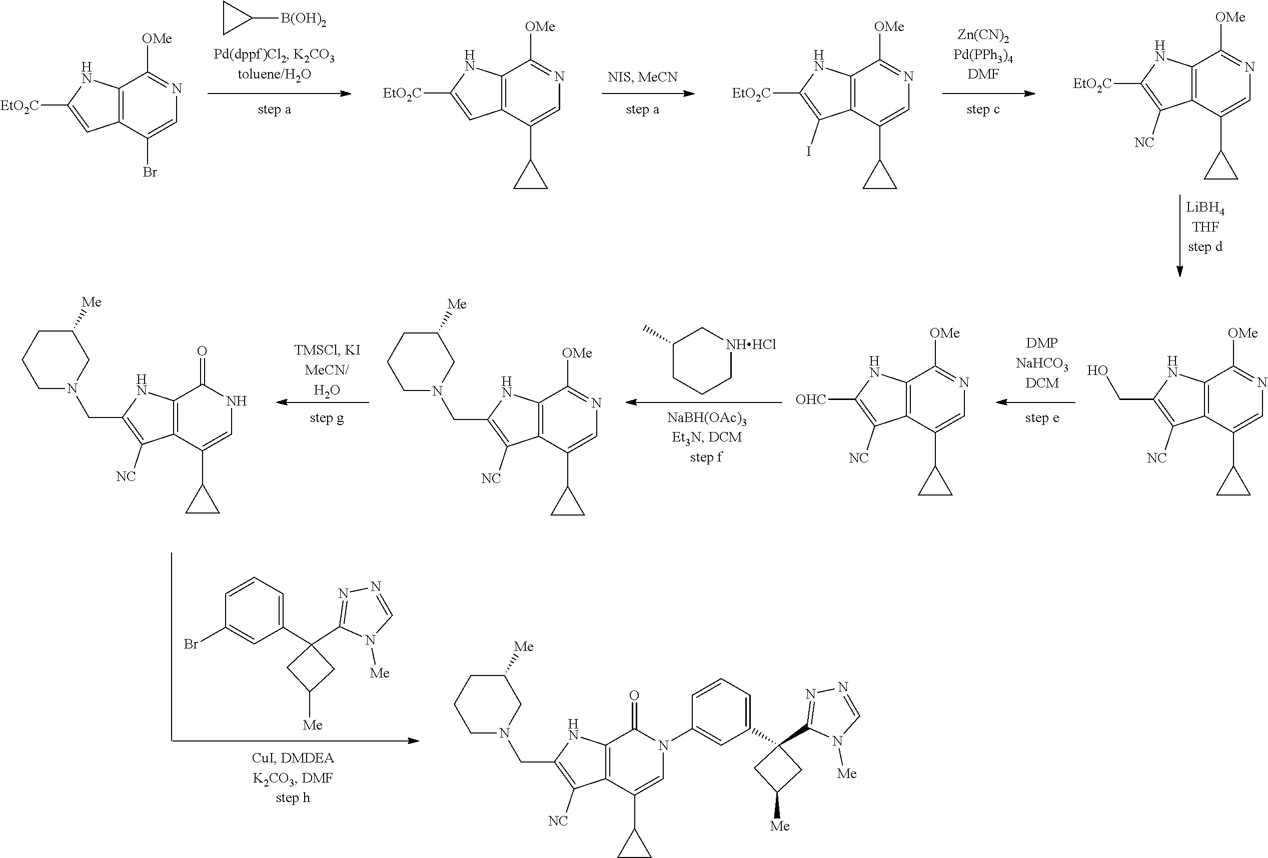

Step a: To a 100-mL round bottom flask under N₂ was added ethyl 4-bromo-7-methoxy-1H-pyrrolo[2,3-c]pyridine-2-carboxylate (1.00 g, 3.4 mmol, 1.0 equiv.), cyclopropylboronic acid (0.584 g, 6.8 mmol, 2.0 equiv.), Pd(dppf)Cl₂ (0.249 g, 0.34 mmol, 10 mol %), K₂CO₃ (1.38 g, 10 mmol, 3.0 equiv.), toluene (10 mL) and H₂O (5 mL). The resulting mixture was heated under reflux for 1 h. After cooling to room temperature, the organic phase was separated, and the aqueous layer was extracted with EtOAc. The combined organic phase was dried over Na₂SO₄, concentrated and the crude residue was purified by column chromatography (SiO₂, EtOAc in hexanes, 0 to 20%) to give the product.

Step b: To a solution of the product from step a (260 mg, 1.0 mmol, 1.0 equiv.) in MeCN (4 mL) was added N-iodosuccinimide (225 mg, 1.1 mmol, 1.1 equiv.). The resulting mixture was stirred for 30 min when LCMS showed the completion of the iodation. The mixture was concentrated, and the crude product was purified by column chromatography (SiO₂, EtOAc in hexanes, 0 to 30%) to give the product ethyl 4-cyclopropyl-3-iodo-7-methoxy-1H-pyrrolo[2,3-c]pyridine-2-carboxylate.

Step c: To a solution of the product from step b (251 mg, 0.65 mmol, 1.0 equiv.) in DMF (2.5 mL) under N₂ was added Zn(CN)₂ (114 mg, 0.97 mmol, 1.5 equiv.) and Pd(PPh₃)₄ (68.6 mg, 0.065 mmol, 10 mol %). The resulting mixture was heated at 110° C. and stirred overnight. After cooling to room temperature, the reaction mixture was diluted with EtOAc, filtered through Celite®, and then washed with H₂O twice. The resulting organic solution was then concentrated, and the crude residue was purified by column chromatography (SiO₂, EtOAc in hexanes, 0 to 30%) to give the product.

Step d: To a solution of the product from step c (62 mg, 0.22 mmol, 1.0 equiv.) in THE (3 mL) was added LiBH₄ (30 mg, 1.4 mmol, 6 equiv.). The resulting mixture was heated at 60° C., stirred overnight, and then quenched with H₂O at room temperature. The organic phase was separated, and the aqueous layer was extracted with EtOAc. The combined organic phase was dried over Na₂SO₄, concentrated, and the crude product was directly applied in the next step.

Step e: To a solution of the product from step d (50.0 mg, 0.21 mmol, 1.0 equiv.) in DCM (2 mL) was added DMP (131 mg, 0.32 mmol, 1.5 equiv.) and NaHCO₃ (35.3 mg, 0.42 mmol, 2.0 equiv.). The resulting mixture was stirred at room temperature for 30 min and quenched with saturated Na₂S₂O₃ and NaHCO₃ aqueous solution. The organic phase was separated, and the aqueous layer was extracted with DCM. The combined organic phase was dried over Na₂SO₄, concentrated and the crude residue was purified by column chromatography (SiO₂, EtOAc in hexanes, 0 to 50%) to give the product.

Step f: To a solution of the product from step e (38.4 mg, 0.16 mmol, 1.0 equiv.) in DCM (1.6 mL) was added (S)-3-methylpiperidine hydrochloride (32.6 mg, 0.24 mmol, 1.5 equiv.) and Et₃N (32.4 mg, 0.32 mmol, 2.0 equiv.). The resulting mixture was stirred at room temperature for 30 min before adding NaBH(OAc)₃ (67.8 mg, 0.32 mmol, 2.0 equiv.). The reaction mixture was then stirred at room temperature for another 30 min, then quenched with H₂O. The organic phase was separated, and the aqueous layer was extracted with DCM twice. The combined organic phase was dried over Na₂SO₄, concentrated, and the crude residue was purified by column chromatography (SiO₂, MeOH in DCM, 0 to 10%) to give the product.

Step g: To a mixture of the product from step f (39.0 mg, 0.12 mmol, 1.0 equiv.) in MeCN/H₂O (4:1 v/v, 2 mL) was added TMSCl (35.0 mg, 0.32 mmol, 2.7 equiv.) and KI (53.1 mg, 0.32 mmol, 2.7 equiv.). The resulting mixture was heated at 80° C. for 1 h when LCMS showed a completion of the demethylation. After cooling to room temperature, the reaction mixture was concentrated to dryness, and the crude product was directly applied in the next step.

Step h: To a solution of the crude product from step g (˜0.12 mmol), and 3-[1-(3-bromophenyl)-3-methylcyclobutyl]-4-methyl-1,2,4-triazole (diastereomeric mixture in 3:1 ratio, 73.5 mg, 0.24 mmol, 2.0 equiv.) in DMF (1.5 mL) was added CuI (22.9 mg, 0.12 mmol, 1.0 equiv.), N,N′-dimethylethylenediamine (21.2 mg, 0.24 mmol, 2.0 equiv.) and K₂CO₃ (49.8 mg, 0.36 mmol, 3.0 equiv.). The resulting mixture was heated at 110° C. for 2 h. After cooling to room temperature, the reaction mixture was diluted with EtOAc and washed with water twice. The organic phase was then washed with brine, dried over Na₂SO₄ and concentrated. The crude residue was purified by HPLC to afford the title compound as a diastereomeric mixture in 5:1 ratio. ¹H NMR (400 MHz, DMSO-d₆) δ 13.10 (br.s, 1H), 8.37 (s, 0.18H), 8.29 (s, 0.84H), 7.55-7.16 (m, 4H), 7.02-6.96 (m, 1H), 3.71 (s, 2H), 3.26 (s, 3H), 3.17-3.08 (m, 0.69H), 2.94-2.82 (m, 1.78H), 2.84-2.72 (m, 2.22H), 2.38-2.21 (m, 0.78H), 2.12-1.90 (m, 2H), 1.77-1.39 (m, 5H), 1.13-1.04 (m, 3H), 0.92-0.77 (m, 6H), 0.76-0.64 (m, 2H). ESI MS [M+H]⁺ for C₃₂H₃₇N₇O, calcd 536.3, found 536.3.

### Example 135: 4-Cyclopropyl-3-methanesulfonyl-2-{[(3S)-3-methylpiperidin-1-yl]methyl}-6-{3-[(1R,3S)-3-methyl-1-(4-methyl-4H-1,2,4-triazol-3-yl)cyclobutyl]phenyl}-1H,6H,7H-pyrrolo[2,3-c]pyridin-7-one

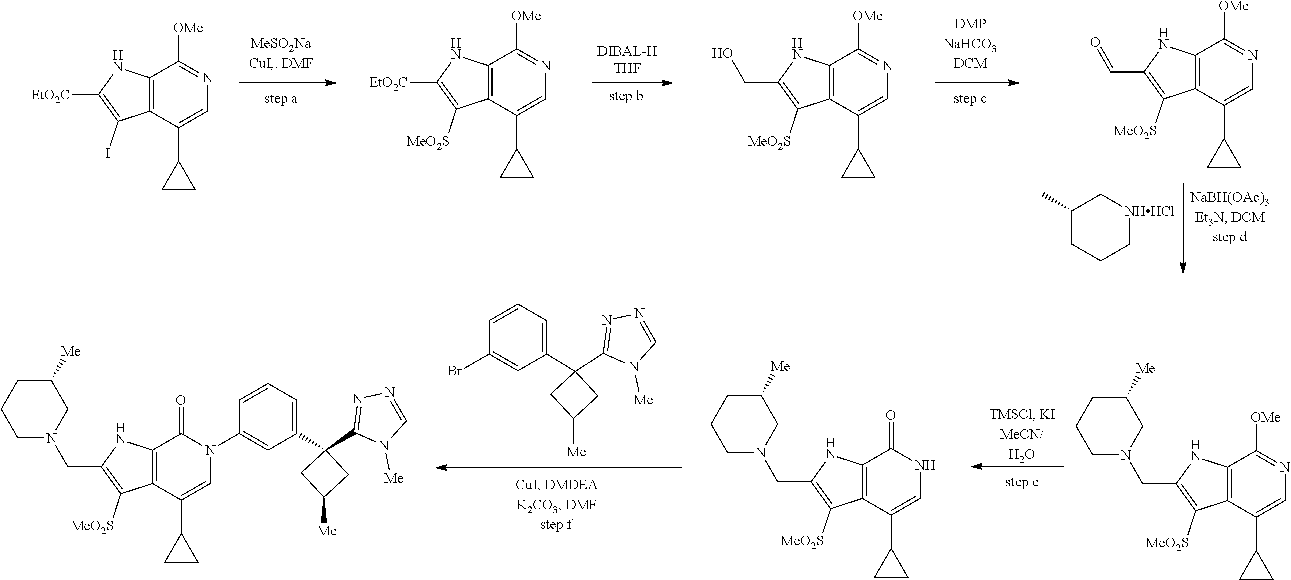

Step a: To a solution of ethyl 4-cyclopropyl-3-iodo-7-methoxy-1H-pyrrolo[2,3-c]pyridine-2-carboxylate (150 mg, 0.39 mmol, 1.0 equiv.) in DMF (3 mL) was added CuI (370 mg, 1.9 mmol, 5.0 equiv.) and sodium methanesulfinate (85% purity, 240 mg, 1.9 mmol, 5.0 equiv.). The resulting mixture was heated at 110° C. for 1 h and then cooled to room temperature. The mixture was diluted with EtOAc and filtered through Celite®. The organic solution was washed with H₂O twice, dried over Na₂SO₄, concentrated, and the resulting crude residue was purified by column chromatography (SiO₂, EtOAc in hexanes, 0 to 50%) to give the product.

Step b: To a solution of the product from step a (65.1 mg, 0.19 mmol, 1.0 equiv.) in THE (1 mL) was added DIBAL-H (1M in toluene, 0.77 mL, 0.77 mmol, 4.0 equiv.) at 0° C. The resulting solution was stirred at room temperature before being quenched with 1 M NaOH aqueous solution. The organic phase was separated, and the aqueous layer was extracted with EtOAc three times. The combined organic phase was dried over Na₂SO₄, concentrated and the crude product was directly applied in the next step.

Step c: To a solution of the product from step b (˜0.19 mmol) in DCM/THF (v/v 1:1, 4 mL) was added DMP (160 mg, 0.40 mmol, 2.0 equiv.) and NaHCO₃ (34.5 mg, 0.40 mmol, 2.0 equiv.). The resulting mixture was stirred at room temperature for 1 h and quenched with saturated Na₂S₂O₃ and NaHCO₃ aqueous solution. The organic phase was separated, and the aqueous layer was extracted with DCM twice. The combined organic phase was dried over Na₂SO₄, concentrated and the crude residue was purified by column chromatography (SiO₂, EtOAc in hexanes, 0 to 50%) to give the product.

Step d: To a solution of the product from step c (100 mg, 0.34 mmol, 1.0 equiv.) in DCM (3.4 mL) was added (S)-3-methylpiperidine hydrochloride (69.1 mg, 0.51 mmol, 1.5 equiv.) and Et₃N (68.8 mg, 0.69 mmol, 2.0 equiv.). The resulting mixture was stirred at room temperature for 30 min before adding NaBH(OAc)₃ (144 mg, 0.68 mmol, 2.0 equiv.). The reaction mixture was then stirred at room temperature for another 30 min before being quenched with H₂O. The organic phase was separated, and the aqueous layer was extracted with DCM twice. The combined organic phase was dried over Na₂SO₄, concentrated and the crude product was directly applied in the next step.

Step e: To a mixture of the product from step d (˜0.34 mmol) in MeCN/H₂O (4:1 v/v, 2 mL) was added TMSCl (55.0 mg, 0.51 mmol, 1.5 equiv.) and KI (85.0 mg, 0.51 mmol, 1.5 equiv.). The resulting mixture was heated at 80° C. for 1 h when LCMS showed a completion of the demethylation. After cooling to room temperature, the reaction mixture was concentrated to dryness, and the crude product was purified by column chromatography (SiO₂, MeOH in DCM, 0 to 10%) to give the product.

Step f: To a solution of the crude product from step e (72.7 mmol, 0.20 mmol, 1.0 equiv.), and 3-[1-(3-bromophenyl)-3-methylcyclobutyl]-4-methyl-1,2,4-triazole (diastereomeric mixture in 3:1 ratio, 91.9 mg, 0.30 mmol, 1.5 equiv.) in DMF (2 mL) was added CuI (38.1 mg, 0.20 mmol, 1.0 equiv.), N,N′-dimethylethylenediamine (35.3 mg, 0.40 mmol, 2.0 equiv.) and K₂CO₃ (82.9 mg, 0.60 mmol, 3.0 equiv.). The resulting mixture was heated at 110° C. for 3 h. After cooling to room temperature, the reaction mixture was diluted with EtOAc and washed with water twice. The organic phase was then washed with brine, dried over Na₂SO₄ and concentrated. The crude residue was purified by HPLC to afford the title compound as a diastereomeric mixture in 3:1 ratio. ¹H NMR (400 MHz, DMSO-d₆) δ 13.14 (br.s, 1H), 8.36 (s, 0.23H), 8.29 (s, 0.77H), 7.59-7.16 (m, 4H), 6.96-6.89 (m, 1H), 3.83 (s, 2H), 3.38 (s, 3H), 3.26 (s, 3H), 3.17-3.08 (m, 0.61H), 2.94-2.77 (m, 1.65H), 2.76-2.65 (m, 2H), 2.62-2.52 (m, 2H), 2.33-2.19 (m, 1H), 1.95 (t, J=11.2 Hz, 1H), 1.72-1.28 (m, 5H), 1.13-0.99 (m, 3H), 0.93-0.73 (m, 6H), 0.72-0.59 (m, 2H). ESI MS [M+H]⁺ for C₃₂H₄₀N₆O₃S, calcd 589.3, found 589.3.

### Example 136: 7-Oxo-6-{3-[(1R,3S)-3-methyl-1-(4-methyl-4H-1,2,4-triazol-3-yl)cyclobutyl]phenyl}-4-(trifluoromethyl)-1H,6H,7H-pyrrolo[2,3-c]pyridine-3-carbonitrile

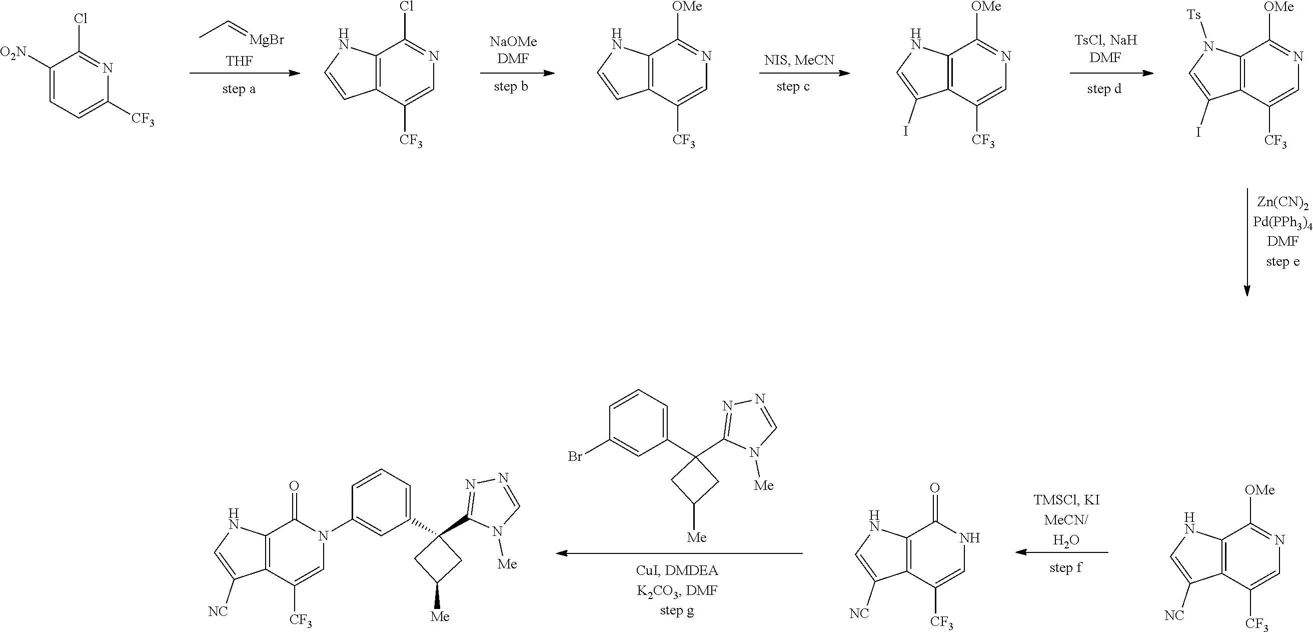

Step a: To a solution of 2-chloro-3-nitro-6-(trifluoromethyl)pyridine (1.00 g, 4.4 mmol, 1.0 equiv.) in THF (15 mL) was added vinylmagnesium bromide (1M in THF, 15 mL, 15 mmol, 3.3 equiv.) at −78° C. over 15 min. The resulting mixture was stirred at this temperature for another 30 min before being quenched with saturated NH₄Cl aqueous solution. The organic phase was separated, and the aqueous layer was extracted with EtOAc twice. The combined organic phase was dried over Na₂SO₄, concentrated, and the crude product was purified by column chromatography (SiO₂, EtOAc in hexanes, 0 to 25%) to give the product.

Step b: To a solution of the product from step a (297 mg, 1.3 mmol, 1.0 equiv.) in DMF (6.5 mL) was added sodium methoxide (702 mg, 13 mmol, 10 equiv.). The resulting mixture was heated at 130° C. for 1 h before being cooled to room temperature and diluted with EtOAc. The organic phase was washed with H₂O twice and brine and then dried over Na₂SO₄, concentrated, and the crude product was purified by column chromatography (SiO₂, EtOAc in hexanes, 0 to 20%) to give the product.

Step c: To a solution of the product from step b (607 mg, 2.8 mmol, 1.0 equiv.) in MeCN (15 mL) was added N-iodosuccinimide (758 mg, 3.4 mmol, 1.2 equiv.) at once. The resulting mixture was stirred for 10 min when LCMS showed the completion of the iodation. The mixture was concentrated, and the crude product was purified by column chromatography (SiO₂, DCM in MeOH, 0 to 10%) to give the product (465 mg, 49% yield).

Step d: To a solution of the product from step c (465 mg, 1.4 mmol, 1.0 equiv.) in DMF (5 mL) was added NaH (60 wt % in mineral oil, 80.0 mg, 2.0 mmol, 1.5 equiv.) at 0° C. The resulting mixture was stirred at this temperature for 10 min before the addition of 4-toluenesulfonyl chloride (381 mg, 2.0 mmol, 1.5 equiv.). The reaction mixture was then raised to room temperature and stirred overnight. The reaction was then quenched with water and diluted with EtOAc. The organic phase was separated and washed with water twice. The organic solution was then washed with brine, dried over Na₂SO₄ and concentrated. The crude residue was purified by column chromatography (SiO₂, EtOAc in hexanes, 0 to 15%) to give 3-iodo-7-methoxy-1-(4-methylphenyl)sulfonyl-4-(trifluoromethyl)pyrrolo[2,3-c]pyridine.

Step e: To a solution of the product from step d (100 mg, 0.20 mmol, 1.0 equiv.) in DMF (2 mL) under N₂ was added Zn(CN)₂ (35.2 mg, 0.30 mmol, 1.5 equiv.) and Pd(PPh₃)₄ (21.1 mg, 0.020 mmol, 10 mol %). The resulting mixture was heated at 110° C. and stirred overnight. After cooling down to room temperature, the reaction mixture was diluted with EtOAc and filtered through Celite®, and then washed with H₂O twice. The resulting organic solution was then concentrated, and the crude residue was purified by column chromatography (SiO₂, MeOH in DCM, 0 to 10%) to give the product.

Step f: To a mixture of the product from step e (62.0 mg, 0.26 mmol, 1.0 equiv.) in MeCN/H₂O (4:1 v/v, 2 mL) was added TMSCl (41.9 mg, 0.39 mmol, 1.5 equiv.) and KI (64.7 mg, 0.39 mmol, 1.5 equiv.). The resulting mixture was heated at 80° C. for 1 h when LCMS showed a completion of the demethylation. After cooling to room temperature, the reaction mixture was concentrated to dryness, and the crude product was purified by column chromatography (SiO₂, MeOH in DCM, 0 to 10%) to afford 7-oxo-4-(trifluoromethyl)-1,6-dihydropyrrolo[2,3-c]pyridine-3-carbonitrile.

Step g: To a solution of the crude product from step f (30.2 mmol, 0.13 mmol, 1.0 equiv.), and 3-[1-(3-bromophenyl)-3-methylcyclobutyl]-4-methyl-1,2,4-triazole (diastereomeric mixture in 3:1 ratio, 61.2 mg, 0.20 mmol, 1.5 equiv.) in DMF (1.3 mL) was added CuI (24.8 mg, 0.13 mmol, 1.0 equiv.), N,N′-dimethylethylenediamine (22.9 mg, 0.26 mmol, 2.0 equiv.) and K₂CO₃ (55.3 mg, 0.40 mmol, 3.0 equiv.). The resulting mixture was heated at 110° C. for 3 h. After cooling to room temperature, the reaction mixture was diluted with EtOAc and washed with water twice. The organic phase was then washed with brine, dried over Na₂SO₄ and concentrated. The crude residue was purified by HPLC to afford the title compound as a diastereomeric mixture in 2:1 ratio.

¹H NMR (400 MHz, CDCl₃) δ 13.09 (s, 1H), 8.15 (s, 0.67H), 8.03 (s, 0.33H), 7.89 (s, 0.62H), 7.62-7.48 (m, 2.3H), 7.42-7.15 (m, 3H), 3.27 (s, 2H), 3.19 (s, 1H), 2.99-2.58 (m, 5H), 1.21-1.09 (m, 3H). ESI MS [M+H]⁺ for C₂₃H₁₉F₃N₆O, calcd 453.2, found 453.2.

### Example 137: 6-{3-[5-(4-Methyl-4H-1,2,4-triazol-3-yl)spiro[2.3]hexan-5-yl]phenyl}-7-oxo-4-(trifluoromethyl)-1H,6H,7H-pyrrolo[2,3-c]pyridine-3-carbonitrile

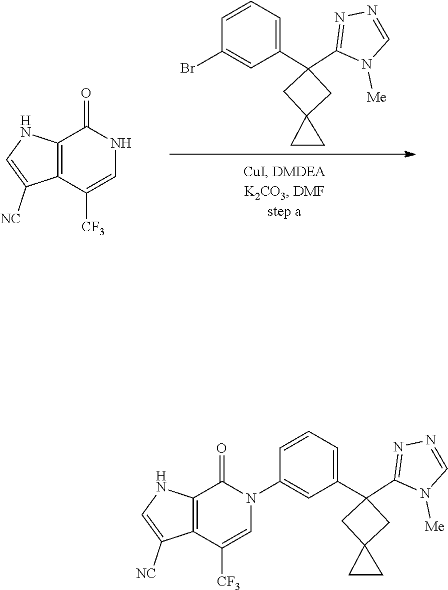 [SMILES: N#Cc1c[nH]c2c(=O)[nH]cc(C(F)(F)F)c12 ; Cn1cnnc1C1(c2cccc(Br)c2)CC2(CC2)C1 ; Cn1cnnc1C1(c2cccc(-n3cc(C(F)(F)F)c4c(C#N)c[nH]c4c3=O)c2)CC2(CC2)C1]

Step a: To a solution of 7-oxo-4-(trifluoromethyl)-1,6-dihydropyrrolo[2,3-c]pyridine-3-carbonitrile (17.6 mg., 0.077 mmol), and 3-[5-(3-bromophenyl)spiro[2.3]hexan-5-yl]-4-methyl-1,2,4-triazole (37.0 mg, 0.12 mmol, 1.5 equiv.) in DMF (1 mL) was added CuI (14.7 mg, 0.077 mmol, 1.0 equiv.), N,N′-dimethylethylenediamine (13.2 mg, 0.15 mmol, 2.0 equiv.) and K₂CO₃ (31.9 mg, 0.23 mmol, 3.0 equiv.). The resulting mixture was heated at 110° C. for 1.5 h. After cooling to room temperature, the reaction mixture was diluted with EtOAc and washed with water twice. The organic phase was then washed with brine, dried over Na₂SO₄ and concentrated. The crude residue was purified by HPLC to afford the title compound. ¹H NMR (400 MHz, Methanol-d4) δ 8.29 (s, 1H), 8.04 (d, J=1.6 Hz, 1H), 7.75 (s, 1H), 7.50 (t, J=7.8 Hz, 1H), 7.40 (s, 1H), 7.36 (d, J=8.1 Hz, 1H), 7.29 (d, J=7.9 Hz, 1H), 3.28 (s, 3H), 3.11 (d, J=11.7 Hz, 2H), 2.77 (d, J=11.6 Hz, 2H), 0.58-0.36 (m, 4H). ESI MS [M+H]⁺ for C₂₄H₁₉F₃N₆O₃, calcd 465.2, found 465.2.

### Example 138 and 139: 3-Methanesulfonyl-6-{3-[(1R,3S)-3-methyl-1-(4-methyl-4H-1,2,4-triazol-3-yl)cyclobutyl]phenyl}-4-(trifluoromethyl)-1H,6H,7H-pyrrolo[2,3-c]pyridin-7-one and 3-Methanesulfonyl-6-{3-[(1S,3R)-3-methyl-1-(4-methyl-4H-1,2,4-triazol-3-yl)cyclobutyl]phenyl}-4-(trifluoromethyl)-1H,6H,7H-pyrrolo[2,3-c]pyridin-7-one

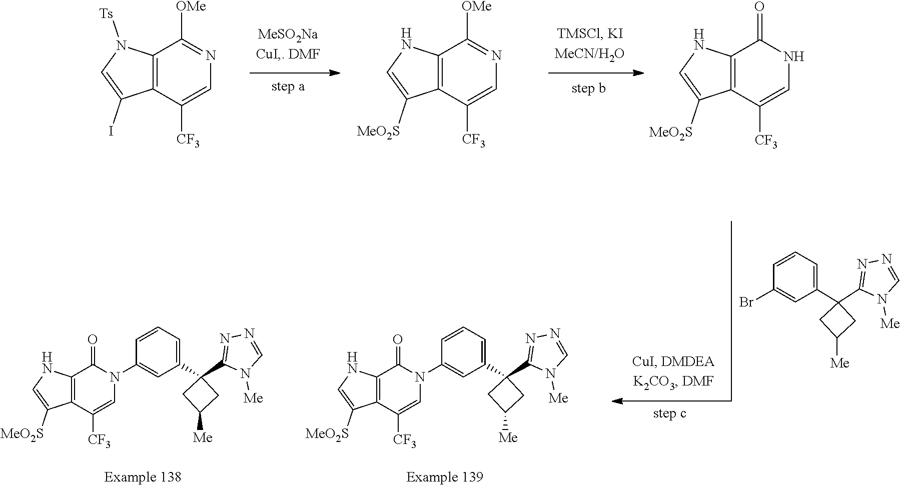 [SMILES: COc1ncc(C(F)(F)F)c2c(I)cn(S(=O)(=O)c3ccc(C)cc3)c12 ; COc1ncc(C(F)(F)F)c2c(S(C)(=O)=O)c[nH]c12 ; CS(=O)(=O)c1c[nH]c2c(=O)[nH]cc(C(F)(F)F)c12 ; Cn1cnnc1[C@]1(c2cccc(-n3cc(C(F)(F)F)c4c(S(C)(=O)=O)c[nH]c4c3=O)c2)C[C@H](C)C1 ; Cn1cnnc1[C@]1(c2cccc(-n3cc(C(F)(F)F)c4c(S(C)(=O)=O)c[nH]c4c3=O)c2)C[C@@H](C)C1 ; CC1CC(c2cccc(Br)c2)(c2nncn2C)C1]

Step a: To a solution of 3-iodo-7-methoxy-1-(4-methylphenyl)sulfonyl-4-(trifluoromethyl)pyrrolo[2,3-c]pyridine (100 mg, 0.20 mmol, 1.0 equiv.) in DMF (2 mL) was added CuI (190 mg, 1.0 mmol, 5.0 equiv.) and sodium methanesulfinate (85% purity, 120 mg, 1.0 mmol, 5.0 equiv.). The resulting mixture was heated at 110° C. for 1 h and then cooled to room temperature. The mixture was diluted with EtOAc and filtered through Celite®. The organic solution was washed with H₂O twice, dried over Na₂SO₄, concentrated, and the resulting crude residue purified by column chromatography (SiO₂, MeOH in DCM, 0 to 10%) to give the product.

Step b: To a mixture of the product from step a (39.4 mg, 0.13 mmol, 1.0 equiv.) in MeCN/H₂O (4:1 v/v, 2 mL) was added TMSCl (27.2 mg, 0.25 mmol, 1.9 equiv.) and KI (41.5 mg, 0.25 mmol, 1.9 equiv.). The resulting mixture was heated at 80° C. for 1 h when LCMS showed a completion of the demethylation. After cooling to room temperature, the reaction mixture was concentrated to dryness, and the crude product was purified by column chromatography (SiO₂, MeOH in DCM, 0 to 10%) to give the product.

Step c: To a solution of the crude product from step b (33.2 mmol, 0.12 mmol, 1.0 equiv.), and 3-[1-(3-bromophenyl)-3-methylcyclobutyl]-4-methyl-1,2,4-triazole (diastereomeric mixture in 3:1 ratio, 55.1 mg, 0.18 mmol, 1.5 equiv.) in DMF (1.2 mL) was added CuI (22.9 mg, 0.12 mmol, 1.0 equiv.), N,N′-dimethylethylenediamine (21.2 mg, 0.24 mmol, 2.0 equiv.) and K₂CO₃ (49.8 mg, 0.36 mmol, 3.0 equiv.). The resulting mixture was heated at 110° C. for 3 h. After cooling to room temperature, the reaction mixture was diluted with EtOAc and washed with water twice. The organic phase was then washed with brine, dried over Na₂SO₄ and concentrated. The crude residue was purified by HPLC to afford the title compounds. Example 138 as a diastereomeric mixture in 5:1 ratio. ¹H NMR (400 MHz, CDCl₃) δ 12.08 (s, 1H), 8.21-7.86 (m, 2H), 7.70-7.53 (m, 3H), 7.40 (s, 1H), 7.35-7.16 (m, 2H), 3.30 (s, 3H), 3.21 (s, 3H), 3.18 (s, 0.6H), 3.17-2.25 (m, 5H), 1.23-1.04 (m, 3H). ESI MS [M+H]⁺ for C₂₃H₂₂F₃N₅O₃S, calcd 506.1, found 506.1. Example 139 as a diastereomeric mixture in 3:1 ratio. ¹H NMR (400 MHz, CDCl₃) δ 11.39 (s, 1H), 8.19-7.90 (m, 2H), 7.79-7.37 (m, 3H), 7.27-7.19 (m, 2H), 3.33 (s, 2H), 3.31-3.15 (m, 6H), 3.00-2.17 (m, 4H), 1.15 (d, J=6.4 Hz, 3H). ESI MS [M+H]⁺ for C₂₃H₂₂F₃N₅O₃S, calcd 506.1, found 506.1.

### Example 140: 6-{3-[(1R,3S)-3-Methyl-1-(4-methyl-4H-1,2,4-triazol-3-yl)cyclobutyl]phenyl}-4-(trifluoromethyl)-1H,6H,7H-pyrazolo[3,4-c]pyridin-7-one

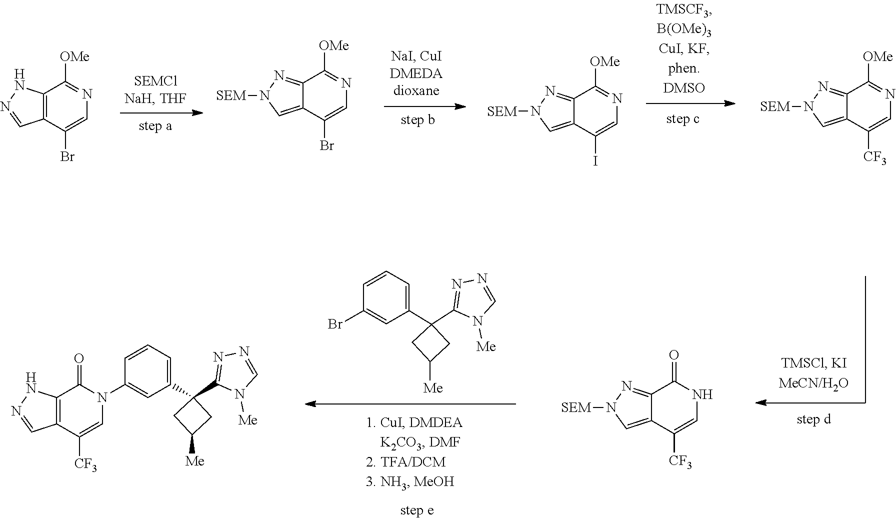

Step a: To a solution of 4-bromo-7-methoxy-1H-pyrazolo[3,4-c]pyridine (1.68 g, 7.4 mmol, 1.0 equiv.) in THF (30 mL) was added NaH (60 wt % in mineral oil, 0.440 g, 11.0 mmol, 1.5 equiv.) at 0° C. The resulting mixture was stirred at this temperature for 10 min before the addition of 2-(trimethylsilyl)ethoxymethyl chloride (1.35 g, 1.4 mL, 8.1 mmol, 1.1 equiv.). The reaction mixture was then raised to room temperature and stirred for 1.5 h. LCMS and TLC showed full conversion to two products as regioisomers. The reaction was then quenched with water and diluted with EtOAc. The organic phase was separated, washed with brine, dried over Na₂SO₄ and concentrated. The crude residue was purified by column chromatography (SiO₂, EtOAc in hexanes, 0 to 25%) to give the desired isomer as product 2-[(4-bromo-7-methoxypyrazolo[3,4-c]pyridin-2-yl)methoxy]ethyl-trimethylsilane.

Step b: To a solution of the product from step a (200 mg, 0.56 mmol, 1.0 equiv.) in dioxane (3 mL) was added NaI (168 mg, 1.1 mmol, 2.0 equiv.), CuI (53.5 mg, 0.28 mmol, 0.50 equiv.) and N,N′-dimethylethylenediamine (48.5 mg, 0.56 mmol, 1.0 equiv.). The resulting mixture was heated at 100° C. under N₂ overnight. After cooling back to room temperature, the reaction mixture was filtered through Celite® and concentrated. The residue was purified by column chromatography (SiO₂, EtOAc in hexanes, 0 to 20%) to give the product.

Step c: To a mixture of the product from step b (38.3 mg, 0.094 mmol, 1.0 equiv.), CuI (9.0 mg, 0.047 mmol, 0.50 equiv.), phenanthroline (8.5 mg, 0.047 mmol, 0.50 equiv.) and KF (16.3 mg, 0.28 mmol, 3.0 equiv.) in DMSO (1 mL) was added trimethyl(trifluoromethyl)silane (39.8 mg, 0.28 mmol, 3.0 equiv.) and trimethyl borate (29.1 mg, 0.28 mmol, 3.0 equiv.). The resulting mixture was heated at 60° C. under N₂ overnight. After cooling back to room temperature, the reaction mixture was diluted with EtOAc and filtered through Celite®. The filtrate was then washed with H₂O three times, dried over Na₂SO₄ and concentrated. The crude residue was purified by column chromatography (SiO₂, EtOAc in hexanes, 0 to 15%) to give the product.

Step d: To a mixture of the product from step c (51.2 mg, 0.15 mmol, 1.0 equiv.) in MeCN/H₂O (4:1 v/v, 2 mL) was added TMSCl (26.1 mg, 0.24 mmol, 1.6 equiv.) and KI (39.8 mg, 0.24 mmol, 1.6 equiv.). The resulting mixture was stirred at room temperature for 2 h when LCMS showed a completion of the demethylation. The reaction mixture was concentrated to dryness, and the crude product was purified by column chromatography (SiO₂, MeOH in DCM, 0 to 10%) to give the product.

Step e: To a solution of the product from step d (44.6 mg, 0.13 mmol, 1.0 equiv.), and 3-[1-(3-bromophenyl)-3-methylcyclobutyl]-4-methyl-1,2,4-triazole (diastereomeric mixture in 3:1 ratio, 61.2 mg, 0.20 mmol, 1.5 equiv.) in DMF (1.3 mL) was added CuI (24.8 mg, 0.13 mmol, 1.0 equiv.), N,N′-dimethylethylenediamine (23.8 mg, 0.27 mmol, 2.0 equiv.) and K₂CO₃ (55.3 mg, 0.40 mmol, 3.0 equiv.). The resulting mixture was heated at 110° C. for 3 h. After cooling to room temperature, the reaction mixture was diluted with EtOAc and washed with water twice. The organic phase was then washed with brine, dried over Na₂SO₄ and concentrated. The residue was then treated with TFA/DCM (v/v 1:10, 2 mL) at room temperature overnight, and then concentrated. The residue was then treated with 7 M NH₃ in methanol (2 mL) for 10 min, followed by concentration under reduced pressure. The crude residue was then purified by HPLC to afford the title compound. ¹H NMR (400 MHz, CDCl₃) δ 8.05 (s, 1H), 8.01 (s, 1H), 7.60-7.41 (m, 4H), 7.31-7.27 (m, 1H), 3.29 (s, 3H), 2.89 (dt, J=7.1, 4.3 Hz, 2H), 2.78-2.60 (m, 3H), 1.15 (d, J=5.2 Hz, 3H). ESI MS [M+H]⁺ for C₂₁H₁₉F₃N₆O, calcd 429.2, found 429.1.

### Example 141: 7-Oxo-6-{3-[(1R,3S)-3-methyl-1-(4-methyl-4H-1,2,4-triazol-3-yl)cyclobutyl]phenyl}-1H,6H,7H-pyrazolo[3,4-c]pyridine-4-carboxylic acid

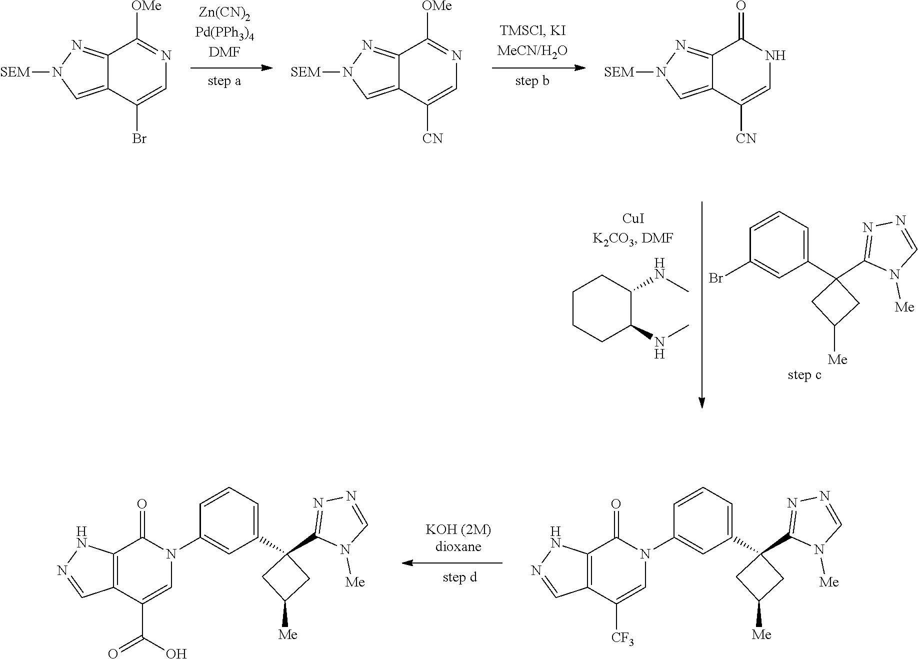

Step a: To a solution of 2-[(4-bromo-7-methoxypyrazolo[3,4-c]pyridin-2-yl)methoxy]ethyl-trimethylsilane (5.0 g, 14 mmol, 1.0 equiv.) in DMF (40 mL) under N₂ was added Zn(CN)₂ (1.97 g, 17 mmol, 1.2 equiv.) and Pd(PPh₃)₄ (0.809 g, 0.70 mmol, 5 mol %). The resulting mixture was heated at 110° C. for 3 h. After cooling down to room temperature, the reaction mixture was diluted with EtOAc, filtered through Celite®, and then washed with H₂O three times. The resulting organic solution was then concentrated, and the crude residue was purified by column chromatography (SiO₂, EtOAc in hexanes, 0 to 15%) to give the product.

Step b: To a mixture of the product from step a (450 mg, 1.5 mmol, 1.0 equiv.) in MeCN/H₂O (4:1 v/v, 6 mL) was added TMSCl (261 mg, 0.30 mL, 2.4 mmol, 1.6 equiv.) and KI (398 mg, 2.4 mmol, 1.6 equiv.). The resulting mixture was stirred at room temperature overnight when LCMS showed a completion of the demethylation. The reaction mixture was concentrated to dryness, and the crude product was purified by column chromatography (SiO₂, EtOAc in hexanes, 20 to 70%) to give the product.

Step c: To a solution of the product from step b (213 mg, 0.73 mmol, 1.0 equiv.), and 3-[1-(3-bromophenyl)-3-methylcyclobutyl]-4-methyl-1,2,4-triazole (diastereomeric mixture in 3:1 ratio, 268 mg, 0.88 mmol, 1.2 equiv.) in DMF (3 mL) was added CuI (28.6 mg, 0.15 mmol, 20 mol %), (1S,2S)-1-N,2-N-dimethylcyclohexane-1,2-diamine (21.3 mg, 0.15 mmol, 20 mol %) and K₂CO₃ (207 mg, 1.5 mmol, 2.0 equiv.). The resulting mixture was heated at 140° C. for 40 h. LCMS showed a full conversion of SEM deprotection. After cooling to room temperature, the reaction mixture was diluted with EtOAc and washed with water twice. The organic phase was then washed with brine, dried over Na₂SO₄ and concentrated. The crude residue was then purified by HPLC to give the product.

Step d: To a solution of the product from step c (18 mg, 0.047 mmol) in dioxane (0.50 mL) was added 2 M KOH aqueous solution (0.50 mL). The resulting mixture was heated at 80° C. overnight. After cooling to room temperature, the reaction mixture was acidified with 6 M HCl (aq, 1 mL) and then concentrated under reduced pressure. The residue was diluted with methanol and filtered to remove precipitates and then concentrated again. The crude material was purified by HPLC to give the title compound as a diastereomeric mixture in 4:1 ratio. ¹H NMR (400 MHz, Methanol-d₄) δ 8.41 (s, 0.8H), 8.26 (s, 0.2H), 8.20 (s, 0.8H), 8.19 (s, 0.2H), 7.98 (t, J=1.8 Hz, 0.2H), 7.82 (dt, J=7.4, 1.5 Hz, 0.2H), 7.74 (s, 0.8H), 7.57 (t, J=1.9 Hz, 0.8H), 7.50-7.26 (m, 3H), 3.24 (s, 2.4H), 3.15 (s, 0.6H), 2.94-2.79 (m, 2H), 2.67-2.53 (m, 1H), 2.54-2.40 (m, 2H), 1.05 (d, J=6.5 Hz, 3H). ESI MS [M+H]⁺ for C₂₁H₂ON₆O₃, calcd 405.2, found 405.2.

### Example 142: 4-(Hydroxymethyl)-6-{3-[(1R,3S)-3-methyl-1-(4-methyl-4H-1,2,4-triazol-3-yl)cyclobutyl]phenyl}-1H,6H,7H-pyrazolo[3,4-c]pyridin-7-one

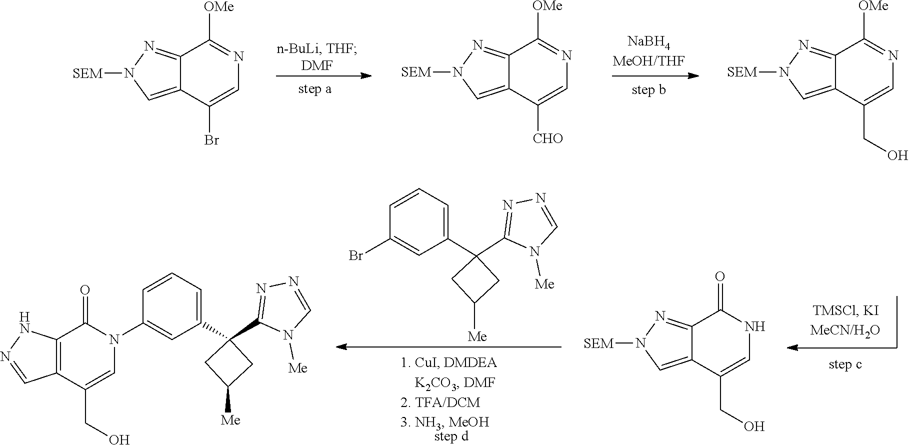 [SMILES: COc1ncc(Br)c2cn(COCC[Si](C)(C)C)nc12 ; COc1ncc(C=O)c2cn(COCC[Si](C)(C)C)nc12 ; COc1ncc(CO)c2cn(COCC[Si](C)(C)C)nc12 ; Cn1cnnc1[C@]1(c2cccc(-n3cc(CO)c4cn[nH]c4c3=O)c2)C[C@H](C)C1 ; CC1CC(c2cccc(Br)c2)(c2nncn2C)C1 ; C[Si](C)(C)CCOCn1cc2c(CO)c[nH]c(=O)c2n1]

Step a: To a solution of 2-[(4-bromo-7-methoxypyrazolo[3,4-c]pyridin-2-yl)methoxy]ethyl-trimethylsilane (500 mg, 1.4 mmol, 1.0 equiv.) in THF (5 mL) was added n-butyllithium (2.5 M in hexanes, 0.67 mL, 1.7 mmol, 1.2 equiv.) at −78° C. The resulting solution was stirred at this temperature for another 30 min, and DMF (0.54 mL, 512 mg, 7.0 mmol, 5.0 equiv.) was added. After another 45-min stirring at −78° C., the reaction mixture was quenched with saturated NH₄Cl aqueous solution and warmed to room temperature. The organic phase was separated, and the aqueous phase was extracted with EtOAc twice. The combined organic solution was then washed with brine, dried over Na₂SO₄ and concentrated. The crude product was directly applied in the next step.

Step b: To a solution of the product from step a in MeOH/THF (v/v 4:1, 5 mL) was added NaBH₄ (110 mg, 2.9 mmol, 2.0 equiv.). The resulting mixture was stirred at room temperature for 10 min and then concentrated directly. The residue was purified by column chromatography (SiO₂, EtOAc in hexanes, 20 to 50%) to give the product.

Step c: To a mixture of the product from step b (244 mg, 0.79 mmol, 1.0 equiv.) in MeCN/H₂O (4:1 v/v, 4 mL) was added TMSCl (0.12 mL, 102 mg, 0.94 mmol, 1.2 equiv.) and KI (157 mg, 0.94 mmol, 1.2 equiv.). The resulting mixture was stirred at room temperature overnight when LCMS showed a completion of the demethylation. The reaction mixture was concentrated to dryness, and the crude product was purified by column chromatography (SiO₂, MeOH in DCM, 0 to 10%) to give the product.

Step d: To a solution of the product from step c (41.3 mg, 0.14 mmol, 1.0 equiv.), and 3-[1-(3-bromophenyl)-3-methylcyclobutyl]-4-methyl-1,2,4-triazole (diastereomeric mixture in 3:1 ratio, 64.3 mg, 0.21 mmol, 1.5 equiv.) in DMF (1.4 mL) was added CuI (26.7 mg, 0.14 mmol, 1.0 equiv.), N,N′-dimethylethylenediamine (18.5 mg, 0.21 mmol, 1.5 equiv.) and K₂CO₃ (58.0 mg, 0.42 mmol, 3.0 equiv.). The resulting mixture was heated at 110° C. for 1 h. After cooling to room temperature, the reaction mixture was diluted with EtOAc and washed with water twice. The organic phase was then washed with brine, dried over Na₂SO₄ and concentrated. The residue was then treated with TFA/DCM (v/v 1:10, 2 mL) at room temperature overnight, and then concentrated. The residue was then treated with 7 M NH₃ in methanol (2 mL) for 10 min, followed by concentration under reduced pressure. The crude residue was then purified by HPLC to afford the title compound. ¹H NMR (400 MHz, Methanol-d₄) δ 8.24 (s, 1H), 7.47 (t, J=7.8 Hz, 1H), 7.43-7.34 (m, 2H), 7.26 (ddd, J=7.8, 2.1, 1.0 Hz, 11H), 7.06 (d, J=7.2 Hz, 1H), 6.77 (d, J=7.2 Hz, 1H), 4.78 (s, 2H), 3.26 (s, 3H), 2.92-2.84 (m, 2H), 2.63-2.42 (m, 3H), 1.04 (d, J=6.1 Hz, 3H).

ESI MS [M+H]⁺ for C₂₁H₂₂N₆O₂, calcd 391.2, found 391.2.

### Example 143: 6—{[(3S)-3-Methylpiperidin-1-yl]methyl}-3-{3-[(1R,3S)-3-methyl-1-(4-methyl-4H-1,2,4-triazol-3-yl)cyclobutyl]phenyl}-7-(trifluoromethyl)-3H,4H,5H-pyrrolo[3,2-d]pyrimidin-4-one

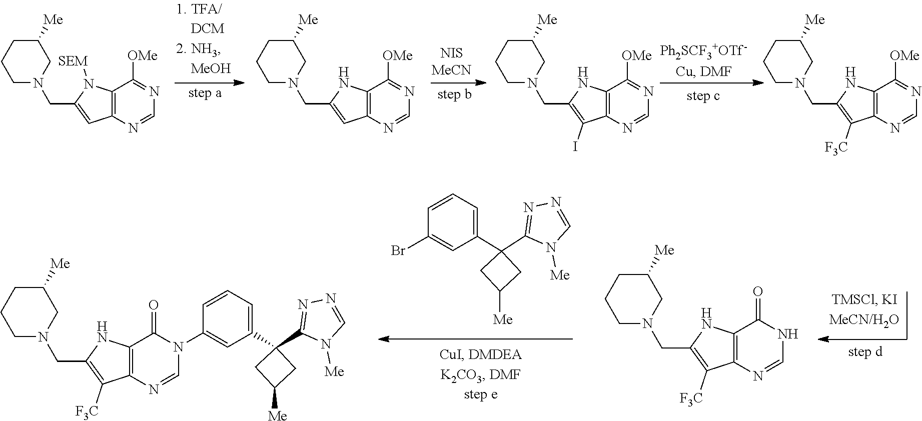

Step a: To a solution of 2-[[4-methoxy-6-[[(3S)-3-methylpiperidin-1-yl]methyl]pyrrolo[3,2-d]pyrimidin-5-yl]methoxy]ethyl-trimethylsilane (200 mg, 0.51 mmol, 1.0 equiv.) in DCM (3 mL) was added TFA (0.30 mL). The resulting solution was stirred at 45° C. overnight before concentration under reduced pressure. The residue was then treated with 7 M NH₃ in methanol (3 mL) for 30 min, followed by concentration under reduced pressure. The crude product was directly applied in the next step.

Step b: To a solution of the product from step a (˜0.51 mmol) in MeCN (3 mL) was added N-iodosuccinimide (225 mg, 1.0 mmol, 2.0 equiv.) at once. The resulting mixture was stirred for 30 min when LCMS showed the completion of the iodation. The mixture was concentrated, and the crude material was purified by column chromatography (SiO₂, DCM in MeOH, 0 to 10%) to give the product.

Step c: To a solution of the product from step b (29.1 mg, 0.075 mmol, 1.0 equiv.) in DMF (0.75 mL) was added diphenyl(trifluoromethyl)sulfonium trifluoromethanesulfonate (93.0 mg, 0.23 mmol, 3.0 equiv.) and copper (24.1 mg, 0.38 mmol, 5.0 equiv.). The resulting mixture was heated at 60° C. for 2 h and then cooled to room temperature and diluted with EtOAc. The mixture was then filtered through Celite®. The filtrate was washed with H₂O twice, dried over Na₂SO₄ and concentrated. The crude product was purified by column chromatography (SiO₂, MeOH in DCM, 0 to 10%) to give the product.

Step d: To a mixture of the product from step c (45.0 mg, 0.14 mmol, 1.0 equiv.) in MeCN/H₂O (4:1 v/v, 1.5 mL) was added TMSCl (23.9 mg, 0.22 mmol, 1.6 equiv.) and KI (49.5 mg, 0.22 mmol, 1.6 equiv.). The resulting mixture was heated at 80° C. for 1 h when LCMS showed a completion of the demethylation. The reaction mixture was concentrated to dryness, and the crude product was purified by column chromatography (SiO₂, MeOH in DCM, 0 to 10%) to give the product.

Step e: To a solution of the product from step d (75.3 mg, 0.20 mmol, 1.0 equiv.), and 3-[1-(3-bromophenyl)-3-methylcyclobutyl]-4-methyl-1,2,4-triazole (diastereomeric mixture in 3:1 ratio, 73.5 mg, 0.24 mmol, 1.2 equiv.) in DMF (2 mL) was added CuI (38.1 mg, 0.20 mmol, 1.0 equiv.), N,N′-dimethylethylenediamine (26.4 mg, 0.30 mmol, 1.5 equiv.) and K₂CO₃ (55.3 mg, 0.40 mmol, 2.0 equiv.). The resulting mixture was heated at 110° C. for 6 h. After cooling to room temperature, the reaction mixture was diluted with EtOAc and washed with water twice. The organic phase was then washed with brine, dried over Na₂SO₄ and concentrated. The residue was then treated with TFA/DCM (v/v 1:10, 4 mL) at room temperature for 4 h, and then concentrated. The residue was then treated with 7 M NH₃ in methanol (4 mL) for 10 min, followed by concentration under reduced pressure. The crude residue was then purified by HPLC to afford the title compound as a diastereomeric mixture in 4:1 ratio. ¹H NMR (400 MHz, CDCl₃) δ 13.66 (s, 1H), 8.17 (s, 1H), 8.05 (s, 1H), 7.59-7.43 (m, 2H), 7.36 (s, 1H), 4.46 (s, 2H), 3.44 (d, J=12.2 Hz, 1H), 3.38-3.24 (m, 4H), 2.95-2.83 (m, 2H), 2.77-2.58 (m, 3H), 2.36 (t, J=11.9 Hz, 1H), 2.20-1.97 (m, 6H), 1.22-1.12 (m, 4H), 0.99 (d, J=6.5 Hz, 3H). ESI MS [M+H]⁺ for C₂₈H₃₃F₃N₇O, calcd 540.3, found 540.2.

### Example 144: 4-cyclopropyl-6-[3-[5-(4-cyclopropyl-1,2,4-triazol-3-yl)spiro[2.3]hexan-5-yl]phenyl]-2-[[(3S)-3-methylpiperidin-1-yl]methyl]-1H-pyrrolo[2,3-c]pyridin-7-one

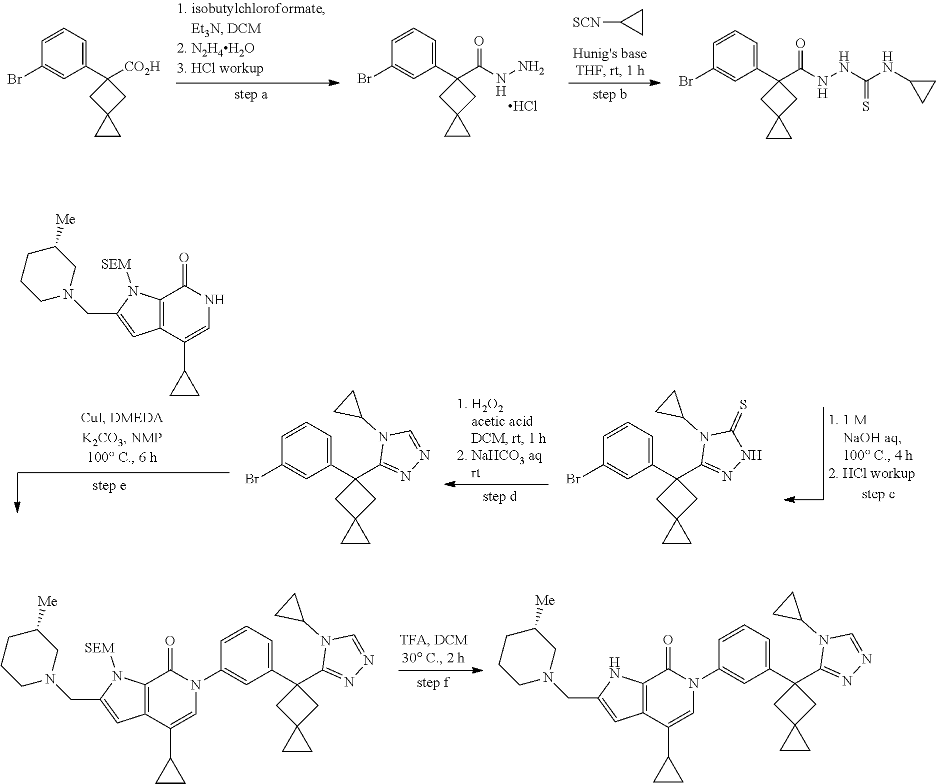

Step a: To a solution of 5-(3-bromophenyl)spiro[2.3]hexane-5-carboxylic acid (2.0 g, 7.11 mmol, 1 equiv.) and triethylamine (1.6 g, 16.35 mmol, 2.3 equiv.) in dichloromethane (30.0 mL) was added isobutyl chloroformate (1.1 g, 8.04 mmol, 1.1 equiv) at 0° C. The reaction was allowed to stir at room temperature for 1 hour. The mixture was then cooled to 0° C., hydrazine hydrate (1.4 g, 25.7 mmol, 55% hydrate, 3.6 equiv.) was added and stirred at room temperature for 1 hour. The reaction mixture was acidified with 1M aq. HCl and to precipitate the product. The white crystalline solid was collected via filtration to yield the desired product that was used for step b without purification.

Step b: To a suspension of the product from step a (500.0 mg, 1.69 mmol, 1 equiv.) in THF (2.0 mL) was added DIPEA (171.0 mg, 1.69 mmol) and cyclopropyl isothiocyanate (337.3 mg, 3.39 mmol, 2 equiv.). The resulting mixture was stirred at room temperature for 1 hour. The volatiles were evaporated under vacuum. The resulting residue was purified by reverse phase column chromatography (C18 modified silica gel, 10-100% acetonitrile/water, 0.1% formic acid) to afford the desired product.

Step c: A suspension of the product from step b (189 mg, 0.48 mmol, 1 equiv.) in 1 M NaOH (10.0 mL) was heated at 100° C. for one hour. After the completion of the reaction, conc. HCl was added to adjust the pH to 1. The white precipitate was collected by filtration to yield the desired product.

Step d: To a solution of the product from step c (148 mg, 0.39 mmol, 1 equiv) in dichloromethane (3.9 mL) was added H₂O₂ (133 μL, 30% solution, 3 equiv.) and AcOH (33 μL). The reaction mixture was stirred at room temperature for 1 hour. Then the reaction was diluted with sat. aq. NaHCO₃ (10.0 mL), and the product was extracted with dichloromethane (3×10 mL). The combined organic extract was dried over MgSO₄ and concentrated to dryness under reduced pressure to produce the corresponding 1,3,4-triazole product that was used for the next step without purification.

Step e: To a solution of the product from step d (103.0 mg, 0.25 mmol, 1 equiv.) and 4-cyclopropyl-2-[[(3S)-3-methylpiperidin-1-yl]methyl]-1-(2-trimethylsilylethoxymethyl)-6H-pyrrolo[2,3-c]pyridin-7-one (111.0 mg, 0.32 mmol, 1.3 equiv.) in NMP (2.4 mL, 0.1 M) was added CuI (47.2 mg, 0.25 mmol, 1 equiv.), DMEA (43.7 mg, 0.50 mmol, 2 equiv.) and K₂CO₃ (102.8 mg, 0.74 mmol, 3 equiv.). The reaction was degassed under vacuum and backfilled with nitrogen 3 times to remove oxygen. Then the resulting solution was stirred at 100° C. for 6 h. The reaction mixture was quenched with aq. NH₄Cl (10.0 mL) and extracted with EtOAc (3×15 mL). The combined organic phase was washed with water 3 times and once with brine, dried over Na₂SO₄ and concentrated to dryness under reduced pressure. The dry residue was fractionated by flash column chromatography (SiO₂, 10-100% acetonitrile/water with 1% formic acid) to furnish the desired compound.

Step f: To a solution of the product from step e (120.0 mg, 0.23 mmol) in dichloromethane (1.1 mL) was added TFA (1.1 mL). The resulting mixture was stirred for 2 hours at room temperature. The reaction mixture was concentrated under reduced pressure, and the crude product was purified by preparative HPLC (20% to 90% acetonitrile/water, 0.1% formic acid) to afford the title compound. [0640]¹H NMR (400 MHz, CDCl₃) δ 9.81 (s, 1H), 8.00 (s, 1H), 7.50-7.40 (m, 2H), 7.39-7.28 (m, 2H), 6.74 (d, J=1.2 Hz, 1H), 6.38 (d, J=1.6 Hz, 1H), 3.59 (d, J=1.9 Hz, 2H), 3.39-3.31 (m, 2H), 2.83-2.65 (m, 5H), 1.98-1.85 (m, 2H), 1.70-1.58 (m, 4H), 0.85 (ddd, J=15.7, 9.0, 5.2 Hz, 8H), 0.70-0.62 (m, 4H), 0.59-0.49 (m, 4H). ESI MS [M+H]⁻ for C₃₄H₄₀N₆O, calcd 549.33, found 549.3.

### Example 145: 4-Cyclopropyl-2-[[(3S)-3-methylpiperidin-1-yl]methyl]-6-[3-[5-(4-propan-2-yl-1,2,4-triazol-3-yl)spiro[2.3]hexan-5-yl]phenyl]-1H-pyrrolo[2,3-c]pyridin-7-one

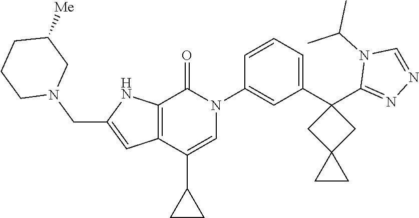 [SMILES: CC(C)n1cnnc1C1(c2cccc(-n3cc(C4CC4)c4cc(CN5CCC[C@H](C)C5)[nH]c4c3=O)c2)CC2(CC2)C1]

The title compound was prepared in a similar fashion to that described for Example 144 by using propan-2-yl thiocyanate in step b. [0642]¹H NMR (400 MHz, CDCl₃) δ 9.61 (s, 1H), 8.18 (s, 1H), 7.45 (t, J=7.8 Hz, 1H), 7.37 (t, J=1.9 Hz, 1H), 7.34-7.27 (m, 2H), 6.69 (d, J=1.2 Hz, 1H), 6.37 (d, J=1.9 Hz, 1H), 3.91 (hept, J=6.7 Hz, 1H), 3.61 (d, J=2.0 Hz, 2H), 3.34-3.25 (m, 2H), 2.85-2.72 (m, 4H), 1.98-1.83 (m, 2H), 1.72-1.52 (m, 5H), 1.17 (d, J=6.7 Hz, 6H), 0.91-0.81 (m, 7H), 0.70-0.48 (m, 6H).

ESI MS [M+H]⁺ for C₃₄H₄₂N₆O, calcd 551.34, found 551.3.

### Example 146: 4-Cyclopropyl-6-[3-[5-[4-(2,2-difluoroethyl)-1,2,4-triazol-3-yl]spiro[2.3]hexan-5-yl]phenyl]-2-[[(3S)-3-methylpiperidin-1-yl]methyl]-1H-pyrrolo[2,3-c]pyridin-7-one

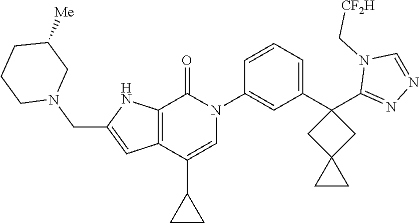 [SMILES: C[C@H]1CCCN(Cc2cc3c(C4CC4)cn(-c4cccc(C5(c6nncn6CC(F)F)CC6(CC6)C5)c4)c(=O)c3[nH]2)C1]

The title compound was prepared in a similar fashion to that described for Example 144 by using 1,1-difluoro-2-isothiocyanatoethane in step b. [0644]¹H NMR (400 MHz, CDCl₃) δ 9.88 (br. s, 1H), 8.21 (s, 1H), 7.52 (t, J=7.7 Hz, 1H), 7.44 (dt, J=8.1, 1.5 Hz, 1H), 7.34-7.22 (m, 2H), 6.71 (d, J=1.3 Hz, 1H), 6.38 (s, 1H), 5.45 (tt, J=54.6, 3.6 Hz, 1H), 3.96 (td, J=14.5, 3.6 Hz, 2H), 3.60 (s, 2H), 3.34-3.24 (m, 2H), 2.88-2.64 (m, 4H), 1.99-1.83 (m, 2H), 1.75-1.46 (m, 6H), 0.96-0.80 (m, 5H), 0.71-0.50 (m, 6H). ¹⁹F NMR (376 MHz, CDCl₃) δ −121.57. ESI MS [M+H]⁺ for C₃₃H₃₈F₂N₆O, calcd 573.31, found 573.3.

### Example 147: 4-Cyclopropyl-2-[[(3S)-3-methylpiperidin-1-yl]methyl]-6-[3-[5-[4-(2,2,2-trifluoroethyl)-1,2,4-triazol-3-yl]spiro[2.3]hexan-5-yl]phenyl]-1H-pyrrolo[2,3-c]pyridin-7-one

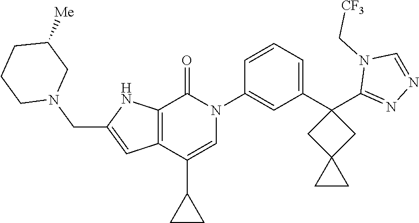 [SMILES: C[C@H]1CCCN(Cc2cc3c(C4CC4)cn(-c4cccc(C5(c6nncn6CC(F)(F)F)CC6(CC6)C5)c4)c(=O)c3[nH]2)C1]

The title compound was prepared in a similar fashion to that described for Example 144 by using 1,1,1-trifluoro-2-isothiocyanatoethane in step b.

¹H NMR (400 MHz, CDCl₃) δ 9.70 (br. s, 1H), 8.25 (s, 1H), 7.62-7.48 (m, 2H), 7.37-7.26 (m, 1H), 7.14 (t, J=2.0 Hz, 1H), 6.71 (d, J=1.2 Hz, 1H), 6.37 (s, 1H), 4.24-4.10 (m, 2H), 3.60 (s, 2H), 3.47-3.33 (m, 2H), 2.86-2.68 (m, 4H), 1.99-1.81 (m, 2H), 1.75-1.43 (m, 6H), 0.92-0.79 (m, 5H), 0.72-0.51 (m, 6H). ¹⁹F NMR (376 MHz, CDCl₃) δ−70.10. ESI MS [M+H]⁻ for C₃₃H₃₈₇F₃N₆O, calcd 591.31, found 591.3.

### Example 148: 2—{[(S)-3-methyl-1-piperidyl]methyl}-4-cyclopropyl-6-(m-{1-[4-(difluoromethyl)-4H-1,2,4-triazol-3-yl]-3-fluorocyclobutyl}phenyl)-1,6-dihydro-1,6-diaza-7-indenone

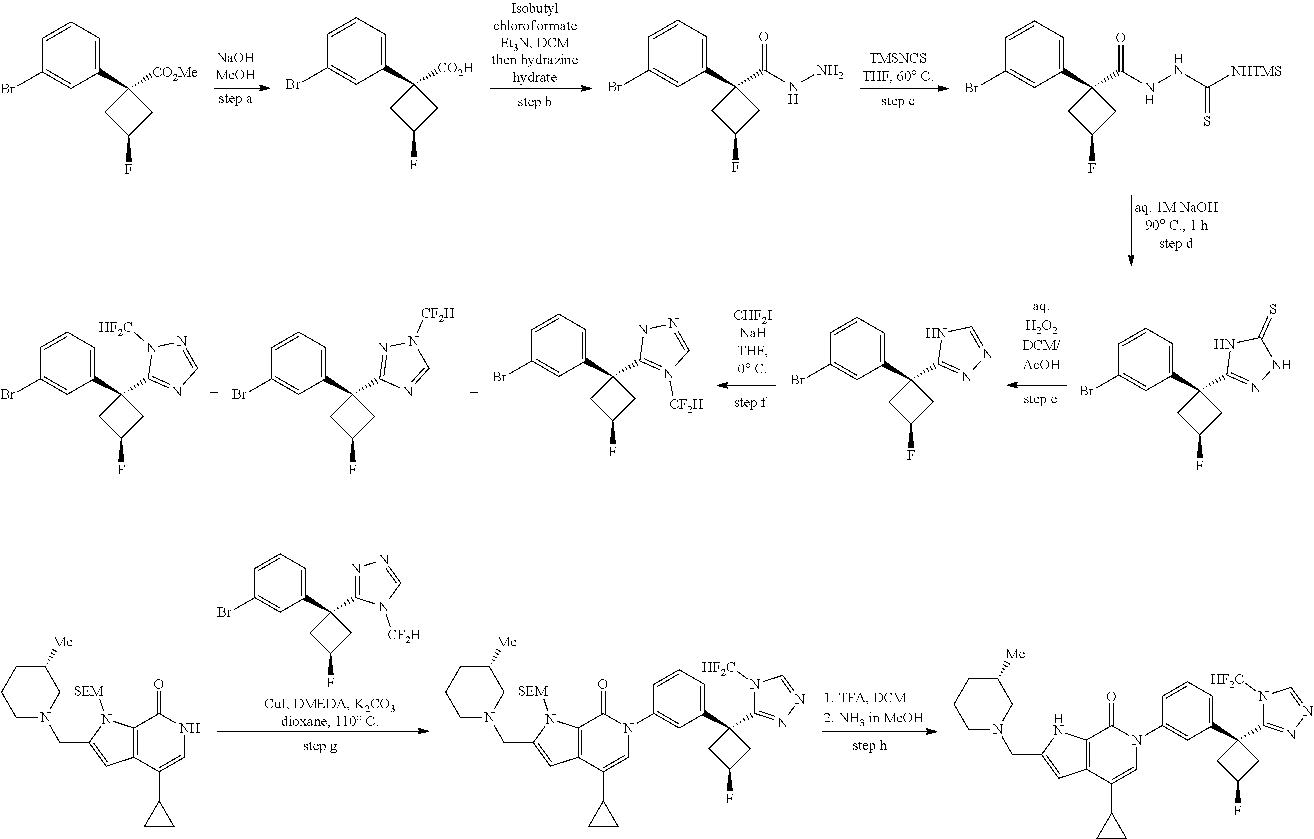

Step a: To a solution of methyl 1-(3-bromophenyl)-3-fluorocyclobutane-1-carboxylate (5.0 g, 17.4 mmol, 1 equiv.) in MeOH/H₂O (90 mL, 1:1 v/v) was added NaOH (3.5 g, 87.4 mmol, 5 equiv.), and the resulting solution was stirred for 2 h at rt. On completion, the reaction mixture was quenched with 1 M HCl, concentrated to remove MeOH, and extracted with EtOAc (3×100 mL). The combined organic phase was washed with brine, dried over Na₂SO₄, and concentrated under reduced pressure. The crude residue was used in step b without further purification.

Step b: To a solution of the product from step a (3.0 g, 11.0 mmol, 1.0 equiv.) in dichloromethane (110 mL, 0.1 M) was added Et₃N (3.5 mL, 25.46 mmol. 2.3 equiv.). The resulting mixture was cooled to 0° C., and isobutyl chloroformate (1.58 mL, 12.17 mmol, 1.1 equiv.) was added dropwise over 1 min. The reaction mixture was allowed to warm and stirred for 1 h at 23° C. The obtained brownish solution was cooled back to 0° C., and hydrazine hydrate (2.7 mL, 44.28 mmol, 4.0 equiv.) was added in one portion. The reaction mixture was allowed to warm to 23° C. and stirred for 30 min. Then it was diluted with dichloromethane and poured into aq. sat. sodium bicarbonate solution (30.0 mL). The organic phase was separated, and the aqueous phase was additionally extracted with dichloromethane (2×75 mL). The combined organic extract was dried over sodium sulfate, and all volatiles were removed under reduced pressure. The crude product obtained upon concentration was used directly in step c.

Step c: The product obtained from step b (11.0 mmol, 3.14 g, 1.0 equiv.) was dissolved in THE, and trimethylsilyl isothiocyanate (0.7 mL, 10.3 mmol, 0.95 equiv.) was added. The resulting mixture was maintained at 60° C. for 2 h. Then it was cooled to room temperature, and all volatiles were removed under reduced pressure. The crude product was purified by reversed-phase column chromatography (C18-modified SiO₂, water/CH₃CN gradient containing with 0.1% formic acid) to produce the desired product.

Step d: The product from step c (2.5 g, 6.1 mmol, 1 equiv.) was dissolved in 30 mL of 1M aq. NaOH and the resulting clear solution was stirred at 90° C. for 30 min. The reaction mixture was cooled to room temperature and acidified with 1M aq. hydrochloric acid to pH˜3. The formed precipitate of the product was extracted with EtOAc (3×50 mL). The combined organic extract was dried over sodium sulfate and concentrated to dryness to produce the cyclized product that was directly used for the next step.

Step e: The product from step d (1.97 g, 6.1 mmol, 1.0 equiv.) was dissolved in a mixture of dichloromethane (27.5 mL) and acetic acid (3.5 mL) and placed in a 100 mL single neck round bottom flask equipped with a magnetic stirring bar. The reaction mixture was cooled to 0° C., and hydrogen peroxide (1.9 mL, 18.57 mmol, 30% aq. solution, 3.0 equiv.) was added. The resulting biphasic mixture was stirred for 20 min, then the cooling bath was removed, and the reaction mixture was maintained at room temperature for an additional 2 h. The mixture was diluted with dichloromethane (30.0 mL), the organic phase was separated, and the aqueous phase was extracted with dichloromethane (2×50 mL). The combined organic extract was washed with sat. aq. NaHCO₃, brine, dried over sodium sulfate, and concentrated to dryness under reduced pressure to produce the 3-[1-(3-bromophenyl)-3-fluorocyclobutyl]-4H-1,2,4-triazole.

Step f: To a solution of the product from step e (410 mg, 1.38 mmol, 1.0 equiv.) in THF (6.9 mL, 0.2 M) was added NaH (166 mg, 60% in mineral oil, 4.16 mmol, 3.0 equiv.) at 0° C. After 20 min, difluoro(iodo)methane (1.03 mL, 1M in CH₃CN 2.07 mmol, 1.5 equiv.) was added to the reaction mixture and stirred for 30 min. On completion, the reaction mixture was quenched with water (10 mL), and the aqueous layer was extracted with EtOAc (3×15 mL). The combined organic phase was dried over Na₂SO₄ and concentrated to dryness on rotary evaporator under reduced pressure. The crude residue was purified by column chromatography (SiO₂, EtOAc in hexanes, 10 to 90%) to give 3-[1-(3-bromophenyl)-3-fluorocyclobutyl]-4-(difluoromethyl)-1,2,4-triazole as the most polar product.

Step g: To a solution of 4-cyclopropyl-2-[[(3S)-3-methylpiperidin-1-yl]methyl]-1-(2-trimethylsilylethoxymethyl)-6H-pyrrolo[2,3-c]pyridin-7-one (75 mg, 0.18 mmol, 1.0 equiv.), 3-[1-(3-bromophenyl)-3-fluorocyclobutyl]-4-(difluoromethyl)-1,2,4-triazole (66 mg, 0.189 mmol, 1.05 equiv.), DMEDA (31.7 μL, 0.36 mmol) in 1,4-dioxane (3 mL), and K₂CO₃ (74.5 mg, 0.54 mmol, 3.0 equiv.) were added in one portion. Nitrogen was bubbled into the solution for 10 mins, followed by adding CuI (35 mg, 0.18 mmol, 1 equiv.). The reaction was stirred in a sealed vial at 110° C. for 2 hours. After cooling to room temperature, sat. aq. NH₄Cl was added, and the product was extracted with dichloromethane (3×10 mL). The combined organic phase was dried over Na₂SO₄, concentrated and the crude residue was purified by column chromatography (SiO₂, 0-10% MeOH/DCM) to give the desired product.

Step h: To a solution of the product from step g (60 mg, 0.088 mmol, 1 equiv.) in DCM (2 mL), TFA (2 mL) was added dropwise, and the reaction was stirred for 1 hour and concentrated under vacuum. The crude product was dissolved in 3.0 mL NH₃ in MeOH and stirred for another 2 hours at room temperature before the crude product was concentrated, and purified by prep-HPLC. ¹H NMR (400 MHz, CDCl₃) δ 9.96 (s, 1H), 8.42 (s, 1H), 7.52 (t, J=7.8 Hz, 1H), 7.42 (d, J=2.0 Hz, 1H), 7.32 (dd, J=7.8, 1.8 Hz, 1H), 7.22 (dd, J=7.9, 1.8 Hz, 1H), 6.97-6.63 (m, 2H), 6.39 (s, 1H), 5.40-5.12 (m, 1H), 3.65-3.44 (m, 4H), 3.02-2.86 (m, 2H), 2.83-2.67 (m, 2H), 1.99-1.81 (m, 2H), 1.73-1.46 (m, 5H), 1.01-0.74 (m, 6H), 0.66 (t, J=5.3 Hz, 2H). ESI MS [M+H]⁺ for C₃₀H₃₃F₃N₆O, calcd 551.26, found 551.20.

### Example 149: 2—{[(S)-3-methyl-1-piperidyl]methyl}-6-(m-{(1r,3S)-1-[4-(difluoromethyl)-4H-1,2,4-triazol-3-yl]-3-fluorocyclobutyl}phenyl)-1,6-dihydro-1,4,6-triaza-7-indenone

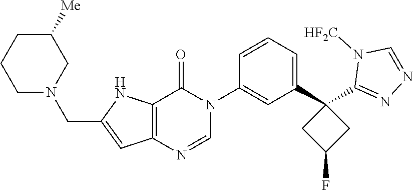

The title compound was prepared in a similar fashion to that described for Example 148 from 6-[[(3S)-3-methylpiperidin-1-yl]methyl]-5-(2-trimethylsilylethoxymethyl)-3H-pyrrolo[3,2-d]pyrimidin-4-one and 3-[1-(3-bromophenyl)-3-fluorocyclobutyl]-4-(difluoromethyl)-1,2,4-triazole. ¹HNMR (400 MHz, CDCl₃) δ 8.43 (s, 1H), 7.95 (s, 1H), 7.59 (t, J=7.9 Hz, 1H), 7.38 (d, J=2.0 Hz, 1H), 7.32 (dd, J=7.9, 2.0 Hz, 2H), 6.88-6.51 (m, 1H), 6.40 (s, 1H), 5.36-5.14 (m, 1H), 3.67 (d, J=3.5 Hz, 2H), 3.60-3.48 (m, 2H), 3.02-2.75 (m, 3H), 2.08-1.99 (m, 1H), 1.79-1.57 (m, 6H), 0.86 (d, J=5.4 Hz, 4H). ESI MS [M+H]⁺ for C₂₆H₂₈F₃N₇O calcd 512.23, found 512.20.

### Example 150: 4-cyclopropyl-6-[3-[5-[4-(difluoromethyl)-1,2,4-triazol-3-yl]spiro[2.3]hexan-5-yl]phenyl]-2-[[(3S)-3-methylpiperidin-1-yl]methyl]-1H-pyrrolo[2,3-c]pyridin-7-one

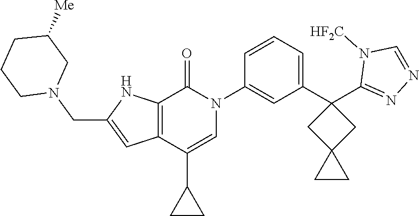

The title compound was prepared in a similar fashion to that described for Example 144 from 3-[5-(3-bromophenyl)spiro[2.3]hexan-5-yl]-4-(difluoromethyl)-1,2,4-triazole and 4-cyclopropyl-2-[[(3S)-3-methylpiperidin-1-yl]methyl]-1-(2-trimethylsilylethoxymethyl)-6H-pyrrolo[2,3-c]pyridin-7-one.

¹H NMR (400 MHz, CDCl₃) δ 9.93 (s, 1H), 8.45 (s, 1H), 7.68-7.40 (m, 2H), 7.40-7.28 (m, 2H), 6.98-6.55 (m, 2H), 6.39 (d, J=1.4 Hz, 1H), 3.60 (d, J=1.8 Hz, 2H), 3.41-3.22 (m, 2H), 2.97-2.63 (m, 4H), 2.02-1.81 (m, 2H), 1.79-1.46 (m, 6H), 1.01-0.76 (m, 7H), 0.69-0.62 (m, 2H), 0.59-0.55 (m, 2H). ¹⁹F NMR (376 MHz, CDCl₃) δ-93.53.

### Example 151: 2—{[(S)-3-methyl-1-piperidyl]methyl}-4-cyclopropyl-6-(m-{(1r,3S)-3-difluoromethoxy-1-[4-(difluoromethyl)-4H-1,2,4-triazol-3-yl]cyclobutyl}phenyl)-1,6-dihydro-1,6-diaza-7-indenone

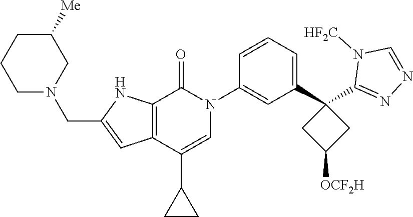

The title compound was prepared in a similar fashion to that described for Example 148 from 3-[1-(3-bromophenyl)-3-(difluoromethoxy)cyclobutyl]-4-(difluoromethyl)-1,2,4-triazole and 4-cyclopropyl-2-[[(3S)-3-methylpiperidin-1-yl]methyl]-1-(2-trimethylsilylethoxymethyl)-6H-pyrrolo[2,3-c]pyridin-7-one. ¹H NMR (400 MHz, CDCl₃) δ 9.67 (s, 1H), 8.44 (s, 1H), 7.51 (t, J=7.9 Hz, 1H), 7.40 (t, J=2.0 Hz, 1H), 7.32-7.27 (m, 1H), 7.22-7.16 (m, 1H), 6.98-6.60 (m, 2H), 6.41-5.92 (m, 2H), 4.94-4.79 (m, 1H), 3.59 (d, J=2.1 Hz, 2H), 3.53-3.44 (m, 2H), 2.95-2.84 (m, 2H), 2.82-2.71 (m, 2H), 1.97-1.84 (m, 2H), 1.75-1.50 (m, 5H), 0.92-0.78 (m, 6H), 0.68-0.61 (m, 2H). ESI MS [M+H]⁻ for C₃₁H₃₄F₄N₆O₂ calcd 599.27, found 599.20.

### Example 152: 4-cyclopropyl-2-({[(1-hydroxycyclobutyl)methyl]amino}methyl)-6-(m-{(1r,3r)-3-difluoromethoxy-1-[4-(difluoromethyl)-4H-1,2,4-triazol-3-yl]cyclobutyl}phenyl)-1,6-dihydro-1,6-diaza-7-indenone

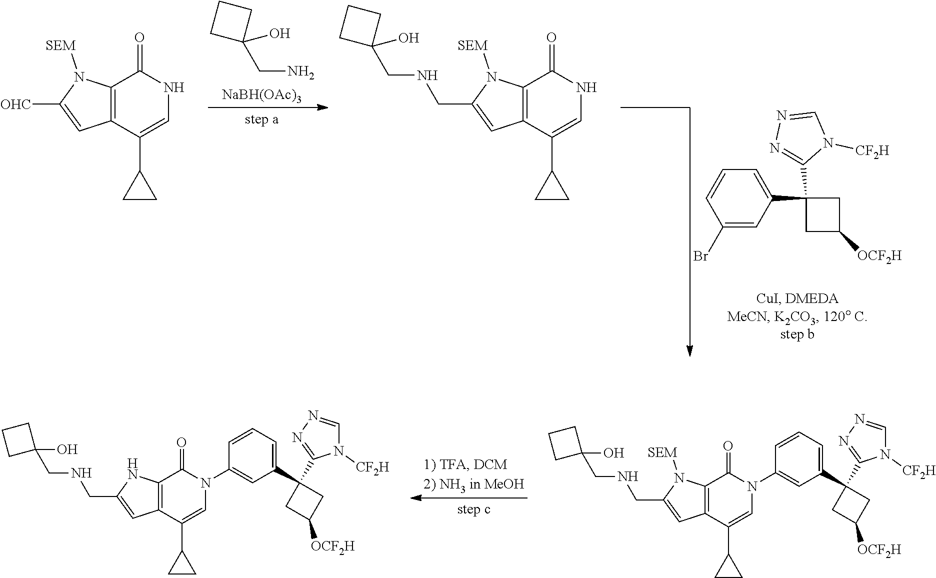

Step a: To a solution of 4-cyclopropyl-7-oxo-1-(2-trimethylsilylethoxymethyl)-6H-pyrrolo[2,3-c]pyridine-2-carbaldehyde (332.2 mg, 1 mmol, 1 equiv.) in dichloromethane (5 mL), 1-(aminomethyl)cyclobutanol (151 mg, 1.5 mmol, 1.5 equiv.) was added. The reaction was stirred at room temperature for 30 mins. Then NaBH(OAc)₃ (423 mg, 2.0 mmol, 2 equiv.) was added and stirred for another 1 hour. Water was added, and the solution was extracted with DCM. The combined organic phase was dried over Na₂SO₄, concentrated and the crude residue was purified by column chromatography (SiO₂, 0-20% MeOH/dichloromethane) to give the desired product.

Step b: The reaction was performed in a similar fashion to Example 148, step g.

Step c: The desired product was prepared in a similar manner to Example 148, step h.

¹H NMR (400 MHz, CDCl₃) δ: 10.83 (s, 1H) 8.43 (s, 1H), 7.50 (t, J=7.9 Hz, 1H), 7.38 (t, J=2.0 Hz, 1H), 7.32-7.28 (m, 1H), 7.23-7.18 (m, 1H), 7.00-6.65 (m, 2H), 6.38 (s, 1H), 6.35-5.93 (m, 1H), 4.95-4.78 (m, 1H), 3.87 (s, 2H), 3.61 (s, 2H), 3.53-3.40 (m, 3H), 2.94-2.80 (m, 2H), 2.00-1.84 (m, 4H), 1.83-1.66 (m, 2H), 0.91-0.83 (m, 2H), 0.66-0.59 (m, 2H). ESI MS [M+H]⁺ for C₃₀H₃₂F₄N₆O₃ calcd 601.25, found 601.20.

### Example 153: 2—{[(S)-3-methyl-1-piperidyl]methyl}-6-(m-{(1r,3S)-3-difluoromethoxy-1-[4-(difluoromethyl)-4H-1,2,4-triazol-3-yl]cyclobutyl}phenyl)-1,6-dihydro-1,4,6-triaza-7-indenone

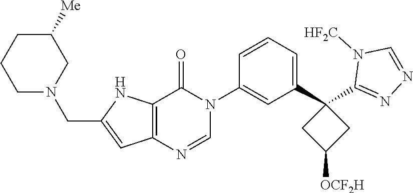

The title compound was prepared in a similar fashion to that described for Example 148 from 3-[1-(3-bromophenyl)-3-(difluoromethoxy)cyclobutyl]-4-(difluoromethyl)-1,2,4-triazole and 6-[[(3S)-3-methylpiperidin-1-yl]methyl]-5-(2-trimethylsilylethoxymethyl)-3H-pyrrolo[3,2-d]pyrimidin-4-one. ¹H NMR (400 MHz, CDCl₃) δ 8.45 (s, 1H), 7.94 (s, 1H), 7.58 (t, J=7.8 Hz, 1H), 7.37 (d, J=2.0 Hz, 1H), 7.34-7.27 (m, 2H), 6.86-6.53 (m, 1H), 6.39 (s, 1H), 6.37-5.96 (m, 1H), 4.93-4.84 (m, 1H), 3.63 (d, J=3.2 Hz, 2H), 3.56-3.47 (m, 2H), 2.95-2.85 (m, 2H), 2.84-2.72 (m, 2H), 2.04-1.95 (m, 1H), 1.76-1.66 (m, 3H), 1.65-1.54 (m, 2H), 0.85 (d, J=5.6 Hz, 4H). ESI MS [M+H]⁺ for C₂₇H₂₉F₄N₇O₂ calcd 560.23, found 560.21.

### Example 154: 6-(m-{(R)-cyclobutyl[4-(difluoromethyl)-4H-1,2,4-triazol-3-yl]methyl}phenyl)-2-{[(S)-3-methyl-1-piperidyl]methyl}-4-cyclopropyl-1,6-dihydro-1,6-diaza-7-indenone

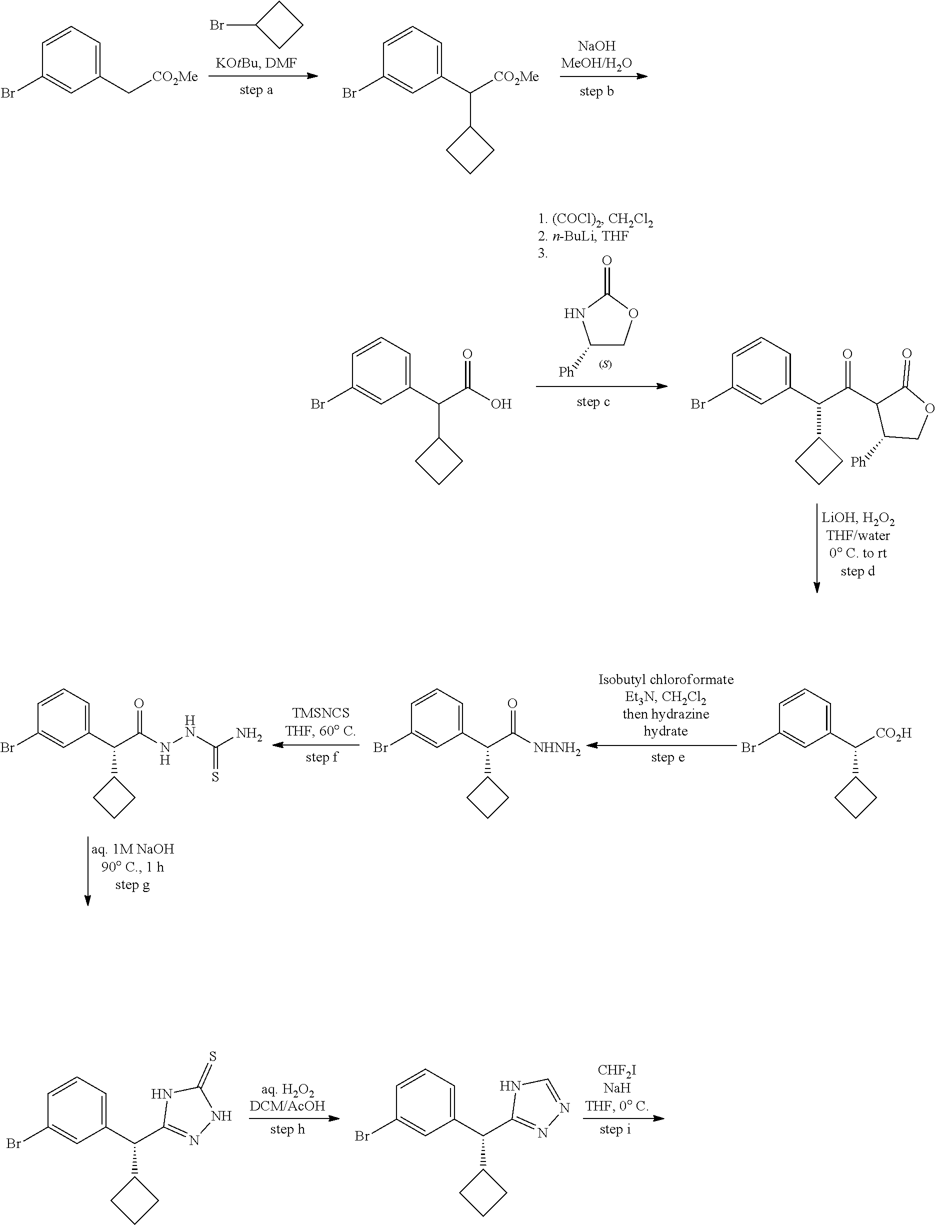

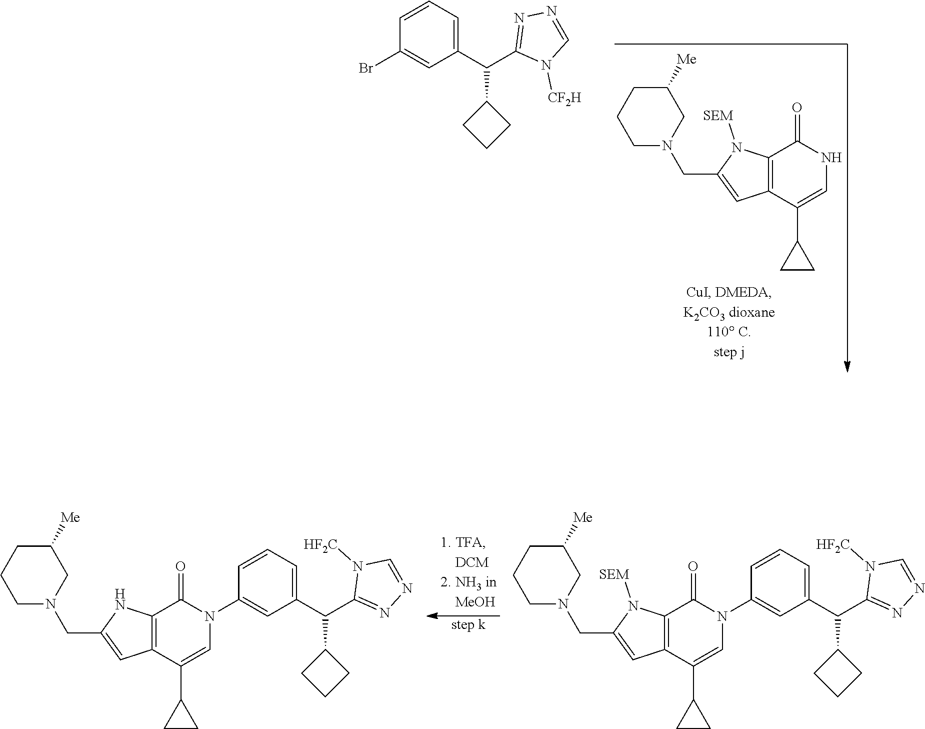

Step a: To a solution of methyl 2-(3-bromophenyl)acetate (19.10 g, 83.4 mmol, 1 equiv.) in DMF (167 mL) was added potassium tert-butoxide (12.16 g, 108.4 mmol, 1.3 equiv.) in portions at 0° C. The reaction mixture was stirred for 30 min before bromocyclobutane (12.4 g, 91.7 mmol, 1.1 equiv.) was added dropwise to the reaction mixture over 5 min. The reaction was allowed to warm to room temperature and stirred overnight. The resulting solution was poured into saturated NH₄Cl (aq) and extracted with EtOAc. The combined organic phase was dried over MgSO₄ and concentrated to dryness under reduced pressure to produce the desired product that was used without further purification.

Step b: To a solution of methyl 2-(3-bromophenyl)-2-cyclobutylacetate (2.00 g, 7.1 mmol, 1 equiv.) in MeOH/H₂O (35.4 mL, v/v=1:1) was added NaOH (1.42 g, 35.5 mmol, 5 equiv.). The resulting mixture was stirred at 50° C. for 2 hours. Upon complete consumption of starting ester the reaction mixture was cooled to room temperature, acidified with aq. 1M HCl, and the product was extracted with dichloromethane. The combined organic phase was dried over MgSO₄ and concentrated under reduced pressure to yield the desired acid.

Step c: To a solution of 2-(3-bromophenyl)-2-cyclobutylacetic acid (8.65 g, 32.14 mmol, 1 equiv.) in dichloromethane (161 mL) at 0° C. was added oxalyl chloride (4.90 g, 38.6 mmol, 1.2 equiv.) and 1 drop of DMF. The reaction was stirred at reflux for 2.5 h. Then it was cooled to room temperature and all volatiles were evaporated under vacuum. The crude acid chloride was dissolved in THE (20.0 mL) and added dropwise over 10 min at −78° C. to a solution obtained by addition of n-BuLi (2.5 M in hexanes, 8.7 mL, 21.9 mmol, 1.1 equiv.) to a solution of (4S)-4-phenyl-1,3-oxazolidin-2-one (3.90 g, 23.9 mmol, 1.2 equiv) in THF (100 mL) at −78° C. The resulting mixture was stirred at −78° C. for 30 min and quenched by addition of aq. sat. NH₄Cl. The product was extracted with EtOAc (3×100 mL), combined organic phase was dried over Na₂SO₄ and concentrated to dryness under reduced pressure. The desired less polar diastereomer was isolated by column chromatography (SiO₂, 0-20% EtOAc/hexanes).

Step d: To a solution of the product from step c (360 mg, 0.9 mmol, 1 equiv.) in THF/H₂O (6.0 mL, v/v=3:1) was added LiOH H₂O (41.6 mg, 1.8 mmol, 2 equiv.) and hydrogen peroxide (4.4 mL, 2 equiv.) and stirred at room temperature for 12 h. The reaction mixture was diluted with water and extracted with MTBE (2×20 mL) to remove impurities. The aqueous layer was acidified with aq. 1 M HCl to pH˜3 and extracted with dichloromethane (3×20 mL). The combined organic extract was dried over MgSO₄ and concentrated to dryness under reduced pressure to yield the desired acid.

Steps e-k were performed in a similar fashion to that described for Example 148 to yield the title compound.

¹H NMR (400 MHz, CDCl₃) δ 8.36 (s, 1H), 7.43 (t, J=7.8 Hz, 1H), 7.36-7.28 (m, 2H), 7.23-7.18 (m, 1H), 7.07-6.74 (m, 1H), 6.71 (s, 1H), 6.37 (s, 1H), 4.08 (d, J=10.5 Hz, 1H), 3.65-3.53 (m, 2H), 3.42-3.28 (m, 1H), 2.82-2.70 (m, 2H), 2.37-2.28 (m, 1H), 2.07-1.76 (m, 7H), 1.75-1.47 (m, 6H), 0.92-0.80 (m, 5H), 0.68-0.60 (m, 2H). ESI MS [M+H]⁺ for C₃₁H₃₆F₂N₆O calcd 547.29, found 547.30.

### Example 155: 6-[3-[cyclobutyl-hydroxy-(4-methyl-1,2,4-triazol-3-yl)methyl]phenyl]-4-cyclopropyl-2-[[(3S)-3-methylpiperidin-1-yl]methyl]-1H-pyrrolo[2,3-c]pyridin-7-one

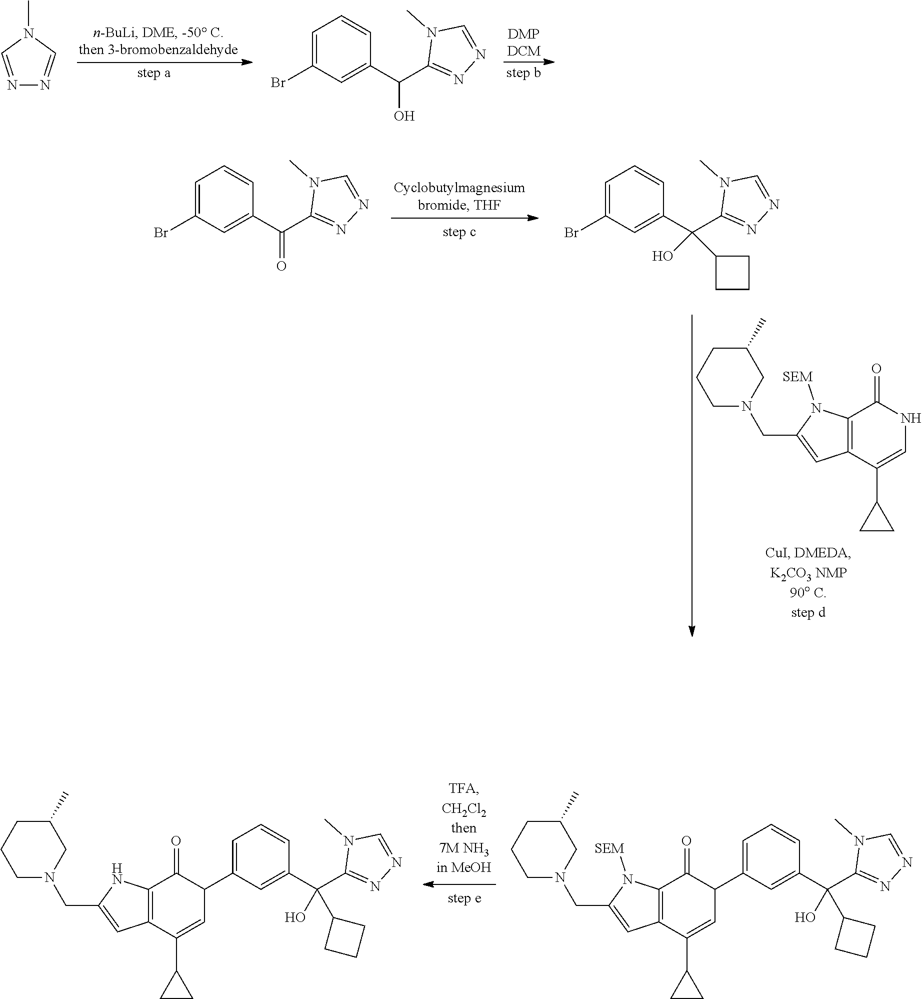

Step a: To a solution of 4-methyl-1,2,4-triazole (0.70 g, 8.42 mmol, 1 equiv.) in dimethoxyethane (42.0 mL) was added n-BuLi (1.6 M in hexanes, 9.26 mmol, 5.8 mL, 1.1 equiv.) at −50° C. and stirred for 1 h. A solution of 3-bromobenzaldehyde (1.71 g, 9.262 mmol, 1.1 equiv.) in dimethoxyethane (10.0 mL) was added dropwise over 5 min. The cooling bath was removed, and the mixture was stirred at ambient temperature for 2 hours. The reaction was quenched with 1 M NaOH (10 mL), and the product was extracted with EtOAc (3×50 mL). The combined organic extract was dried over MgSO₄, concentrated and the crude residue was purified by reversed phase column chromatography (C18-modified SiO₂, water/CH₃CN gradient containing with 0.1% formic acid) to furnish the desired compound.

Step b: To a suspension of the product from step a (770.0 mg, 2.87 mmol, 1 equiv.) in dichloromethane (30 mL) Dess-Martin periodinane (1.8 g, 4.31 mmol, 1.5 equiv.) was added at room temperature. After stirring for 3 hours the reaction was diluted with dichloromethane (20 mL) and quenched with aq. Na₂S₂O₃ (10.0 mL) and sat. aq. NaHCO₃ (10.0 mL). The resulting biphasic mixture was stirred for 1 hour. The product was extracted with dichloromethane (3×10 mL). The combined organic extract was sequentially washed with brine (10 mL) and water (10 mL), dried over MgSO₄ and concentrated to dryness under reduced pressure to produce the crude product that was used for the next step without purification.

Step c: To a solution of the product from step b (200 mg, 0.75 mmol, 1 equiv.) in dichloromethane (7.5 mL) was added cyclobutylmagnesium bromide (3.75 mL, 3.75 mmol, 5.0 equiv., 1.0 M in Et₂₀) at 0° C. The reaction was stirred at 0° C. for 1.5 h followed by quench with sat. aq. NH₄Cl (10 mL). The product was extracted with EtOAc (3×10 mL). The combined organic extract was dried over MgSO₄ and concentrated to dryness under reduced pressure. The dry residue was purified by flash column chromatography (C18-modified SiO₂, water/CH₃CN gradient containing with 0.1% formic acid) to furnish the desired compound.

Step d: To a solution of the product from step c (77.0 mg, 0.19 mmol, 1 equiv.) and 4-cyclopropyl-2-[[(3S)-3-methylpiperidin-1-yl]methyl]-1-(2-trimethylsilylethoxymethyl)-6H-pyrrolo[2,3-c]pyridin-7-one (71.0 mg, 0.22 mmol, 1 equiv.) in NMP (1.9 mL, 0.1 M) was added CuI (35.0 mg, 0.19 mmol, 1 equiv.), DMEDA (33.0 mg, 0.37 mmol, 2 equiv.) and K₂CO₃ (77 mg, 0.56 mmol, 3 equiv). The resulting solution was stirred at 90° C. for 16 h. The reaction was quenched with H₂O and extracted with EtOAc (3×5 mL). The combined organic phase was washed with water (2×10 mL), dried over MgSO₄ and concentrated under reduced pressure. The crude residue was purified by flash column chromatography (SiO₂, 10-100% acetonitrile/water with 1% formic acid) to furnish the desired compound.

Step e: To a solution of the product from step d (70.0 mg, 0.11 mmol) in dichloromethane (1.0 mL) was added TFA (1.0 mL), and the reaction was stirred for 17 hour and concentrated under vacuum. The crude product was dissolved in 2.0 mL 7M NH₃ in MeOH and stirred for another 1 hour at room temperature before the crude product was concentrated, and the crude residue was purified by flash column chromatography (C18-modified SiO₂, water/CH₃CN gradient containing with 0.1% formic acid). ¹H NMR (400 MHz, CDCl₃) δ 9.85 (d, J=12.3 Hz, 1H), 7.93 (s, 1H), 7.45 (q, J=2.0 Hz, 1H), 7.39 (t, J=7.8 Hz, 1H), 7.30-7.25 (m, 1H), 7.22 (d, J=1.6 Hz, 1H), 6.69 (d, J=1.2 Hz, 1H), 6.35 (d, J=1.7 Hz, 1H), 3.72-3.57 (m, 3H), 3.39 (s, 3H), 2.77 (dd, J=17.4, 9.3 Hz, 2H), 2.23 (dt, J=7.4, 3.8 Hz, 1H), 2.11-1.80 (m, 6H), 1.75-1.50 (m, 7H), 0.90-0.75 (m, 6H), 0.69-0.58 (m, 2H). ESI MS [M+H]⁺ for C₃₁H₃₈N₆O₂H, calcd 527.3, found 527.3.

### Example 156: 6-[3-[1-cyclobutyl-2,2,2-trifluoro-1-(4-methyl-1,2,4-triazol-3-yl)ethyl]phenyl]-4-cyclopropyl-2-[[(3S)-3-methylpiperidin-1-yl]methyl]-1H-pyrrolo[2,3-c]pyridin-7-one

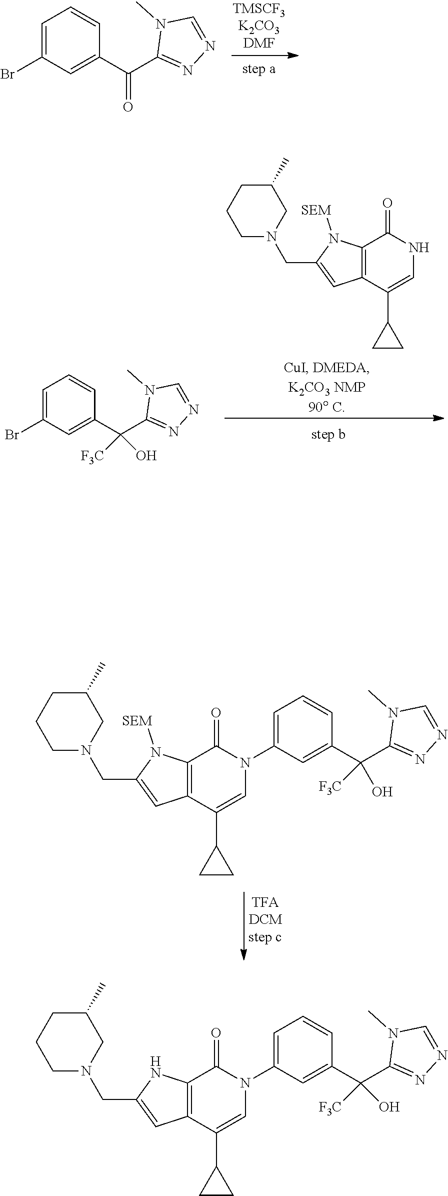 [SMILES: Cn1cnnc1C(=O)c1cccc(Br)c1 ; C[C@H]1CCCN(Cc2cc3c(C4CC4)c[nH]c(=O)c3n2COCC[Si](C)(C)C)C1 ; Cn1cnnc1C(O)(c1cccc(Br)c1)C(F)(F)F ; C[C@H]1CCCN(Cc2cc3c(C4CC4)cn(-c4cccc(C(O)(c5nncn5C)C(F)(F)F)c4)c(=O)c3n2COCC[Si](C)(C)C)C1 ; C[C@H]1CCCN(Cc2cc3c(C4CC4)cn(-c4cccc(C(O)(c5nncn5C)C(F)(F)F)c4)c(=O)c3[nH]2)C1]

Step a: To a solution of (3-bromophenyl)-(4-methyl-1,2,4-triazol-3-yl)methanone (65.0 mg, 0.25 mmol, 1.0 equiv.) in DMF (2.4 mL, 0.1 M) was added TMSCF₃ (53 mg, 0.29 mmol, 1.2 equiv.) and K₂CO₃ (5.0 mg, 0.0024 mmol, 0.01 equiv.). The reaction mixture was stirred at room temperature overnight. Once complete conversion was observed by TLC analysis the mixture was diluted with H₂O (10 mL) and extracted with EtOAc (3×10 mL). The combined organic phase was washed with water (2×20 mL), dried over MgSO₄ and concentrated to dryness under reduced pressure. The crude residue was purified by flash column chromatography (SiO₂, 0 to 10% methanol in dichloromethane) to furnish the desired compound.

Step b: To a solution of the product from step a (47.1 mg, 0.11 mmol, 1 equiv.) and 4-cyclopropyl-2-[[(3S)-3-methylpiperidin-1-yl]methyl]-1-(2-trimethylsilylethoxymethyl)-6H-pyrrolo[2,3-c]pyridin-7-one (38.0 mg, 0.11 mmol, 1 equiv.) in NMP (1.3 mL, 0.1 M) was added CuI (22.0 mg, 0.11 mmol, 1 equiv.), DMEDA (20.0 mg, 0.22 mmol, 2 equiv.) and K₂CO₃ (47.0 mg, 0.34 mmol, 3 equiv.). The resulting mixture was stirred at 90° C. for 16 h. The reaction was quenched with H₂O and extracted with EtOAc (3×5 mL). The combined organic phase was dried over MgSO₄, concentrated and the crude residue was purified by flash column chromatography (C18-modified SiO₂, water/CH₃CN gradient containing with 0.1% formic acid) to furnish the desired compound.

Step c: To a solution of the product from step b (40.0 mg, 0.06 mmol) in dichloromethane (3.0 mL) was added trifluoroacetic acid (2.0 mL). The reaction mixture was stirred for 2 hours at 35° C. and concentrated to dryness under reduced pressure. The crude residue was purified by flash column chromatography (C18-modified SiO₂, water/CH₃CN gradient containing with 0.1% formic acid). ¹H NMR (400 MHz, CDCl₃) δ 11.65 (s, 1H), 7.96 (s, 1H), 7.70 (s, 2H), 7.51 (d, J=9.1 Hz, 1H), 7.36 (d, J=6.3 Hz, 1H), 6.76 (s, 1H), 6.37 (s, 1H), 3.71 (d, J=15.9 Hz, 6H), 2.74 (s, 1H), 2.09-1.64 (m, 7H), 0.90-0.60 (m, 15H). ¹⁹F NMR (376 MHz, CDCl₃) δ-74.34. ESI MS [M+H]⁺ for C₃₂H₃₇F₃N₆OH, calcd 541.3, found 541.2.

### Example 157: 2—{[(S)-3-methyl-1-piperidyl]methyl}-4-cyclopropyl-6-{4-[1-(4-methyl-4H-1,2,4-triazol-3-yl)ethyl]-2-pyridyl}-1,6-dihydro-1,6-diaza-7-indenone

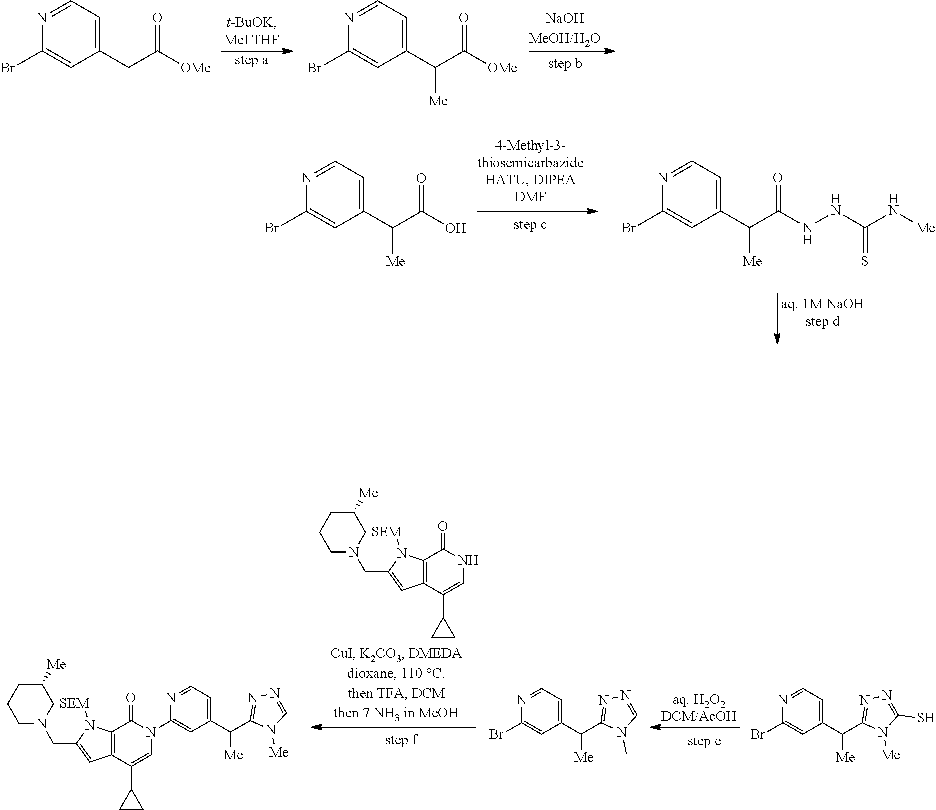

Step a: To a solution of methyl 2-(2-bromopyridin-4-yl)acetate (0.5 g, 2.16 mmol) in THF (11 mL, 0.2 M) was added t-BuOK (0.265 g, 2.37 mmol, 1.1 equiv) at 0° C., and the resulting mixture stirred for 30 min. MeI (0.306 g, 2.16 mmol, 1.0 equiv.) was added, and the reaction was stirred at 0° C. for 1.5 h. Once complete conversion was observed by LCMS and TLC analysis the mixture was quenched with water (10 mL). The product was extracted with EtOAc (3×15 mL). The combined organic phase was dried over Na₂SO₄, concentrated, and the crude residue was purified by column chromatography (SiO₂, EtOAc in hexanes, 10 to 70%) to yield the desired product.

Step b: To a solution of the product from step a (0.25 g, 1.02 mmol) in MeOH (2.5 ml) and H₂O (2.5 mL) was added NaOH (82 mg, 2.04 mmol, 2.0 equiv.) at room temperature. The resulting solution was stirred for 4 hours. Once complete hydrolysis was observed by TLC analysis the reaction mixture was quenched with 1 M HCl, and the product was extracted with EtOAc (3×15 mL). The combined organic phase was washed with brine, dried over Na₂SO₄, and concentrated to dryness under reduced pressure. The crude residue was used in next step without further purification.

Step c: To a solution of the product from step b (0.23 g, 1.02 mmol) in DMF (15 mL), DIPEA (0.26 mL, 1.53 mmol, 1.5 equiv.), HATU (0.5 g, 1.32 mmol, 1.3 equiv.) and 4-methyl-3-thiosemicarbazide (0.16 g, 1.5 equiv.) were added. The reaction mixture was stirred overnight at room temperature. Then it was diluted with EtOAc (30 mL), the organic phase was separated, sequentially washed with water (20 mL) and brine (20 mL), dried over Na₂SO₄ and concentrated to dryness under reduced pressure. The crude product was used in the next step without further purification.

Step d: The product from step c (1.02 mmol, 1.0 equiv.) was dissolved in 4 mL of 1M aq. NaOH, and the resulting clear solution was stirred at 60° C. for 2 h. The reaction mixture was cooled to room temperature and acidified with 1M aq. HCl to pH˜3. The product was extracted with EtOAc (3×15 mL). The combined organic extract was dried over sodium sulfate and concentrated to dryness under reduced pressure to produce the desired product that was directly used for the next step.

Step e: The product from step d (1.02 mmol, 1.0 equiv.) was dissolved in a mixture of dichloromethane (4 mL) and acetic acid (0.5 mL). The resulting mixture was cooled to 0° C., and hydrogen peroxide (0.3 mL, 3.0 mmol, 30% aq. solution, 3.0 equiv.) was added. The resulting biphasic mixture was vigorously stirred for 20 min, then the cooling bath was removed, and the reaction mixture was maintained at room temperature for an additional 2 h under stirring. The reaction was diluted with dichloromethane (20 mL), the organic phase was separated, and the aqueous phase was additionally extracted with dichloromethane (2×50 mL). The combined organic phase was dried over Na₂SO₄, and concentrated. The crude residue was purified by column chromatography (C18-modified SiO₂, water/CH₃CN gradient containing with 0.1% formic acid) to give the desired product.

Step f: A solution of the product from step e (35 mg, 0.13 mmol, 1.1 equiv.), 4-cyclopropyl-2-[[(3S)-3-methylpiperidin-1-yl]methyl]-1-(2-trimethylsilylethoxymethyl)-6H-pyrrolo[2,3-c]pyridin-7-one (50 mg, 0.13 mmol, 1.0 equiv.), DMEDA (50 μL, 0.24 mmol, 2.0 equiv.) and K₂CO₃ (50.9 mg, 0.363 mmol, 3.0 equiv.) in 1,4-dioxane (3 mL) was degassed by sparging with nitrogen for 10 mins. CuI (23 mg, 0.12 mmol, 1.0 equiv) was added, and the resulting mixture was heated at 110° C. for 3 h. The reaction mixture was cooled to room temperature, diluted with EtOAc (20 mL) and sequentially washed with aq. NH₄Cl (15 mL) and water (2×20 mL). The organic phase was then washed with brine, dried over Na₂SO₄ and concentrated to dryness under reduced pressure. The crude residue was then treated with TFA/dichloromethane mixture (v/v 1:1, 2 mL) at room temperature for 4 h and concentrated. The residue was then treated with 7 M NH₃ in methanol (2 mL) for 1 h, followed by concentration under reduced pressure. The crude residue was then purified by RP HPLC (C18 modified SiO₂, water/CH₃CN gradient containing with 0.1% formic acid) to afford the title compound. ¹H NMR (400 MHz, CDCl₃) δ 9.61 (s, 1H), 8.49 (d, J=5.2, 0.7 Hz, 1H), 8.09 (s, 1H), 7.85-7.81 (m, 1H), 7.31 (d, J=1.2 Hz, 1H), 7.10 (dd, J=5.2, 1.6 Hz, 1H), 6.37 (s, 1H), 4.25 (q, J=7.1 Hz, 1H), 3.63-3.58 (m, 2H), 3.50 (s, 3H), 2.85-2.72 (m, 2H), 1.88 (d, J=7.1 Hz, 5H), 1.74-1.52 (m, 5H), 0.93-0.79 (m, 6H), 0.72-0.65 (m, 2H). ESI MS [M+H]⁺ for C₂₇H₃₃N₇ calcd 472.28, found 472.30.

### Example 158: 4-cyclopropyl-6-[4-[methoxy-(4-methyl-1,2,4-triazol-3-yl)methyl]pyridin-2-yl]-2-[[(3S)-3-methylpiperidin-1-yl]methyl]-1H-pyrrolo[2,3-c]pyridin-7-one

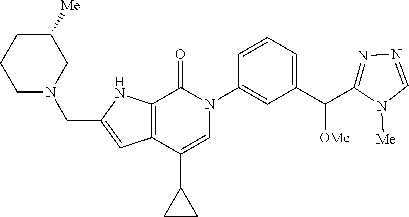 [SMILES: COC(c1cccc(-n2cc(C3CC3)c3cc(CN4CCC[C@H](C)C4)[nH]c3c2=O)c1)c1nncn1C]

The title compound was prepared similarly to example 157 starting from methyl 2-(3-bromophenyl)-2-methoxyacetate. ¹H NMR (400 MHz, CDCl₃) δ 9.80 (br. s, 1H), 8.05 (s, 1H), 7.52 (s, 1H), 7.50-7.41 (m, 1H), 7.37-7.29 (m, 2H), 6.72 (s, 1H), 6.48-6.24 (m, 1H), 5.89 (s, 1H), 3.59 (s, 2H), 3.52 (s, 3H), 3.47 (s, 3H), 2.84-2.67 (m, 2H), 1.97-1.81 (m, 2H), 1.75-1.47 (m, SH), 0.94-0.74 (m, 6H), 0.69-061 (m, 2H). ESI MS [M+H]⁻ for C₂₈H₃₄N₆O₂ calcd 487.28, found 487.30.

### Example 159: 4-cyclopropyl-6-[3-[1-methoxy-1-(4-methyl-1,2,4-triazol-3-yl)ethyl]phenyl]-2-[[(3S)-3-methylpiperidin-1-yl]methyl]-1H-pyrrolo[2,3-c]pyridin-7-one

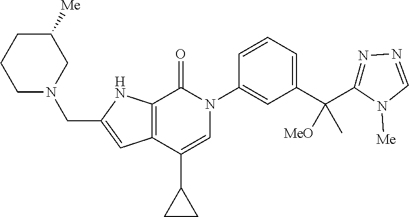 [SMILES: COC(C)(c1cccc(-n2cc(C3CC3)c3cc(CN4CCC[C@H](C)C4)[nH]c3c2=O)c1)c1nncn1C]

The title compound was prepared similarly to example 157 starting from methyl 2-(3-bromophenyl)-2-methoxypropanoate. 1H NMR (400 MHz, CDCl₃) δ 9.76 (br. s, 1H), 8.07 (s, 1H), 7.49-7.38 (m, 2H), 7.34-7.26 (m, 1H), 7.26-7.22 (m, 1H), 6.71 (d, J=1.2 Hz, 1H), 6.37 (s, 1H), 3.59 (d, J=2.1 Hz, 2H), 3.36 (s, 3H), 3.24 (s, 3H), 2.78 (t, J=12.4 Hz, 2H), 2.06 (s, 3H), 1.96-1.80 (m, 2H), 1.74-1.47 (m, 5H), 1.00-0.76 (m, 6H), 0.69-0.55 (m, 2H). ESI MS [M+H]⁺ for C₂₉H₃₆N₆O₂ calcd 501.30, found 501.30.

### Example 160: 2—{[(S)-3-methyl-1-piperidyl]methyl}-4-cyclopropyl-6-{4-[1-methyl-1-(4-methyl-4H-1,2,4-triazol-3-yl)ethyl]-2-pyridyl}-1,6-dihydro-1,6-diaza-7-indenone

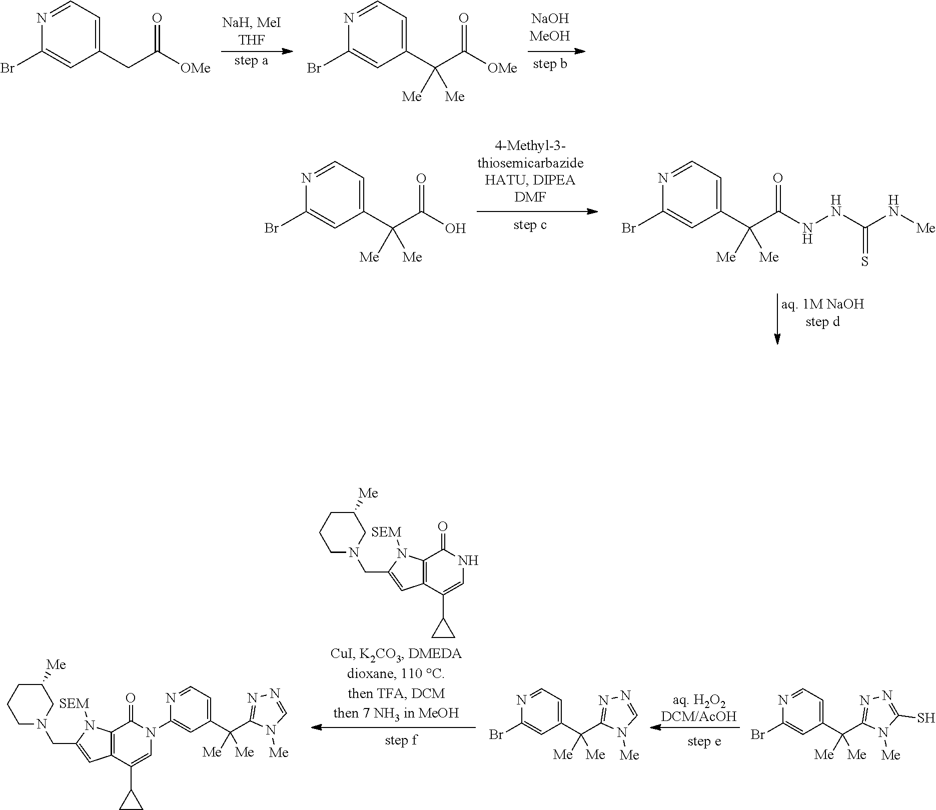

Step a: To a solution of methyl 2-(2-bromopyridin-4-yl)acetate (0.75 g, 3.26 mmol) in THF (0.2 M, 15 mL) was added NaH (0.33 g, 8.15 mmol, 2.5 equiv, 60% in mineral oil) at 0° C. and the resulting mixture stirred for 20 min. Then, MeI (2.1 mL g, 2.16 mmol, 10.0 equiv.) was added to the reaction mixture at 0° C. After 10 min the cooling bath was removed, and the mixture was allowed to stir at ambient temperature overnight. The reaction was diluted with EtOAc (30 mL) and quenched with aq. sat. NH₄Cl (15 ml). The organic phase was separated, and the aqueous phase was additionally extracted with EtOAc (2×15 mL). The combined organic phase was dried over Na₂SO₄ and concentrated to dryness under reduced pressure. The dry residue was purified by column chromatography (SiO₂, EtOAc in hexanes, 10 to 70%) to afford the desired product.

Step b-f: The steps b-f were performed in a similar fashion to example 157. ¹H NMR (400 MHz, CDCl₃) δ 8.52 (d, J=5.3 Hz, 1H), 8.09 (s, 1H), 7.72 (d, J=1.7 Hz, 1H), 7.28 (d, J=1.2 Hz, 1H), 7.07 (dd, J=5.3, 1.8 Hz, 1H), 6.43 (s, 1H), 3.76 (s, 2H), 3.32 (s, 3H), 3.01-2.83 (m, 2H), 1.95-1.82 (m, 8H), 1.79-1.61 (m, 5H), 0.87-0.85 (m, 6H), 0.71-0.66 (m, 2H). ESI MS [M+H]⁺ for C₂₈H₃₅N₇ calcd 486.64, found 486.29.

### Example 161: 4-cyclopropyl-6-[3-[difluoro-(4-methyl-1,2,4-triazol-3-yl)methyl]phenyl]-2-[[(3S)-3-methylpiperidin-1-yl]methyl]-1H-pyrrolo[2,3-c]pyridin-7-one

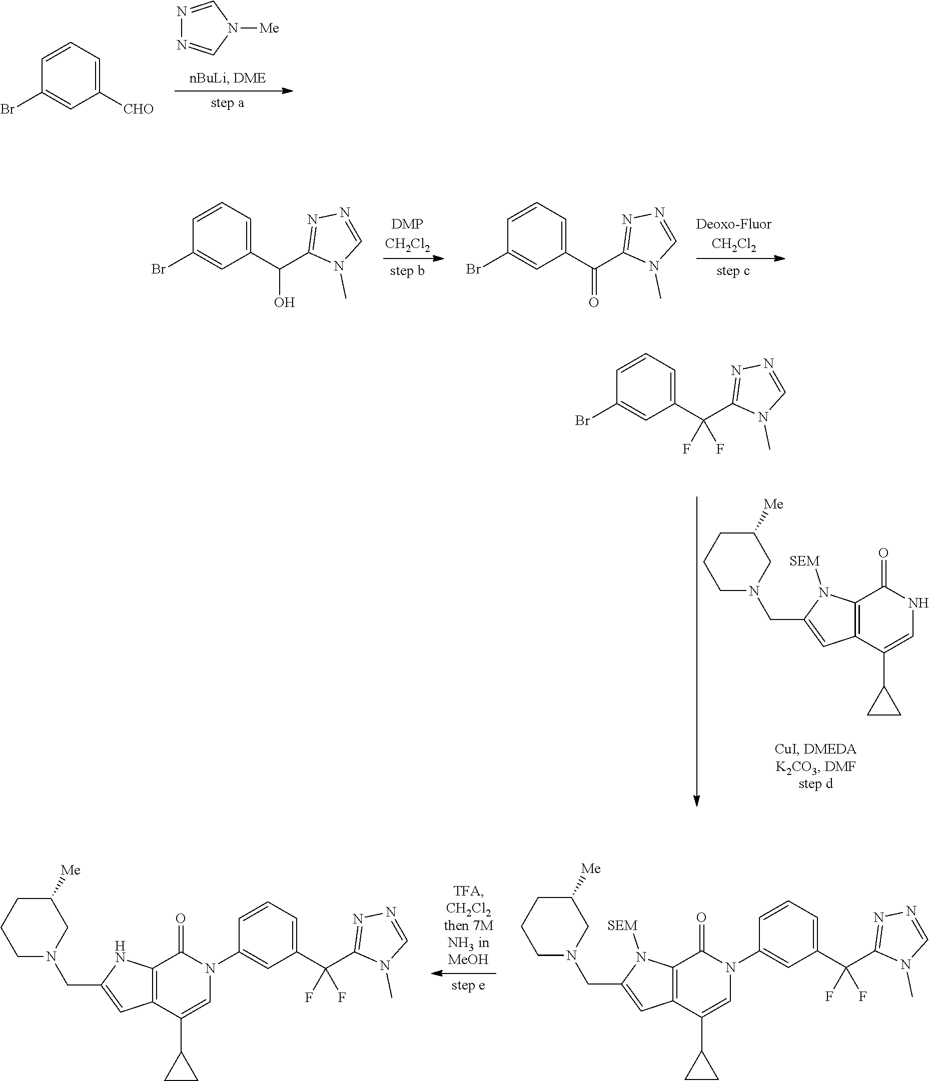

Step a: n-BuLi (2.4 mL, 6.0 mmol, 1 equiv.) was added dropwise to a solution of 4-methyl-4H-1,2,4-triazole (0.5 g, 6.0 mmol, 1 equiv.) in DME (20 mL, 0.3 M) at −40° C. The reaction mixture was stirred at this temperature for 1 h followed by the addition of 3-bromobenzaldehyde (0.92 g, 5.0 mmol). After another hour at −40° C. the mixture was quenched with water (20 mL), and the product was extracted with chloroform/isopropanol mixture (2×20 mL, 3:1, v/v). The combined organic extract was dried over Na₂SO₄ and concentrated to dryness under reduced pressure. The crude residue was purified by column chromatography (SiO₂, 0-10% MeOH in dichloromethane gradient) to give the desired product.

Step b: To a solution of product from step a (0.27 g, 1.0 mmol, 1 equiv.) in dichloromethane (8 mL) Dess-Martin periodinane (424 mg, 2.0 mmol, 1 equiv.) was added. The resulting mixture was stirred at room temperature overnight. The reaction was quenched with aq. sat. Na₂S₂O₃ (10 mL) and stirred for 0.5 h. The product was extracted with chloroform/isopropanol mixture (2×20 mL, 3:1, v/v). The combined organic extract was dried over Na₂SO₄, concentrated and the crude residue was purified by column chromatography (SiO₂, 0-100% EtOAc in dichloromethane gradient) to afford the desired ketone.

Step c: Deoxo-Fluor® (0.22 g, 1 mmol, 3 equiv.) was added to a solution of the product from step b (95 mg, 0.36 mmol, 1 equiv.) in dichloromethane (3 mL) at 0° C. The resulting mixture was stirred at 0° C. for 30 min than allowed to warm to room temperature and refluxed overnight. The resulting solution was quenched with water, and the product was extracted with chloroform/isopropanol mixture (2×10 mL, 3:1, v/v). The combined organic extract was dried over Na₂SO₄, concentrated to dryness under reduced pressure, and the crude residue was purified by column chromatography (SiO₂, 0-10% MeOH in dichloromethane gradient) to afford the desired product.

Step d: To a solution of the product from step c (39.0 mg, 0.14 mmol, 1.4 equiv.), 4-cyclopropyl-2-[[(3S)-3-methylpiperidin-1-yl]methyl]-1-(2-trimethylsilylethoxymethyl)-6H-pyrrolo[2,3-c]pyridin-7-one (41.5 mg, 0.1 mmol, 1 equiv.) and DMEDA (21 μL, 0.2 mmol, 2.8 equiv.) in DMF (2 mL), K₂CO₃ (41.4 mg, 0.3 mmol, 3 equiv.) was added in one portion. The reaction mixture was sparged with nitrogen for 10 mins, then CuI (19.0 mg, 0.1 mmol, 1 equiv.) was added. The resulting mixture was stirred in sealed vial at 100° C. overnight. After cooling to room temperature, saturated NH₄Cl (5 mL) was added. The crude product was extracted with EtOAc (3×15 mL). The combined organic extract was washed with water (15 mL) and brine (15 mL), dried over Na₂SO₄ and concentrated to dryness under reduced pressure. The dry residue was purified by column chromatography (SiO₂, 0-5% MeOH in dichloromethane gradient) to yield the desired product.

Step e: To a solution of the product from step d (40 mg, 0.08 mmol) in dichloromethane (1.0 mL) trifluoroacetic acid (1.0 mL) was added dropwise at 0° C. The reaction mixture was warmed to room temperature and stirred for 1 h. After complete consumption of the starting material was observed by LCMS analysis the solvent was evaporated under reduced pressure. The dry residue was dissolved in 7 M NH₃ in MeOH (1 mL), and the resulting solution was stirred for 1 h at room temperature. Upon concentration the product was isolated by RP HPLC (C18 modified SiO₂, water/CH₃CN gradient containing with 0.1% formic acid). ¹H NMR (400 MHz, CDCl₃) δ 8.18 (s, 1H), 7.69 (s, 1H), 7.66 (t, J=6.6 Hz, 1H), 7.64-7.59 (m, 2H), 6.78 (d, J=1.1 Hz, 1H), 6.41 (s, 1H), 3.88 (s, 3H), 3.69 (s, 2H), 2.86 (dd, J=20.5, 10.1 Hz, 2H), 2.00 (q, J=9.5, 7.9 Hz, 1H), 1.90-1.84 (m, 3H), 1.74-1.58 (m, 4H), 0.88-0.79 (m, 6H), 0.68-0.59 (m, 2H). ESI MS [M+H]⁺ for C₂₇H₃₀F₂N₆O, calcd 492.2, found 492.1.

### Example 162: 4-cyclopropyl-6-[3-[fluoro-(4-methyl-1,2,4-triazol-3-yl)methyl]phenyl]-2-[[(3S)-3-methylpiperidin-1-yl]methyl]-1H-pyrrolo[2,3-c]pyridin-7-one

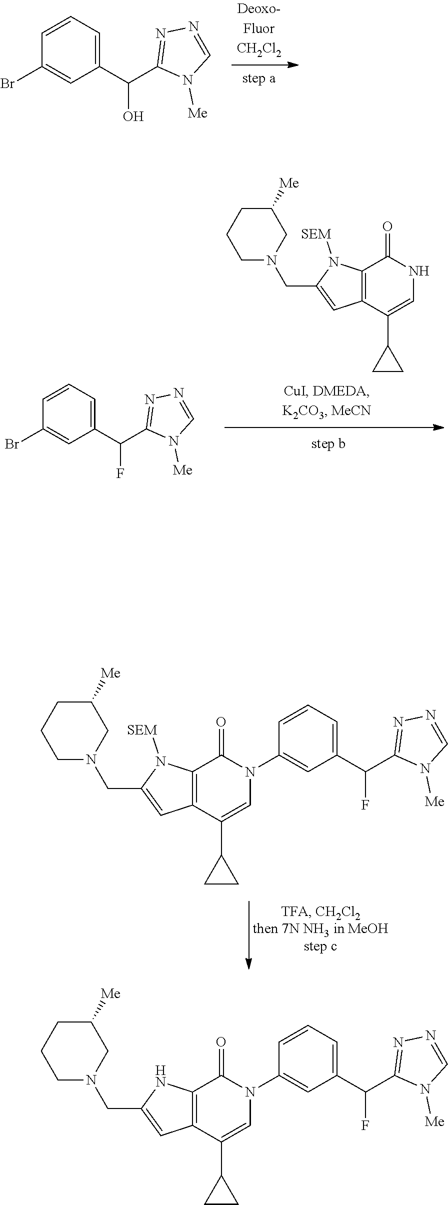 [SMILES: Cn1cnnc1C(O)c1cccc(Br)c1 ; C[C@H]1CCCN(Cc2cc3c(C4CC4)c[nH]c(=O)c3n2COCC[Si](C)(C)C)C1 ; Cn1cnnc1C(F)c1cccc(Br)c1 ; C[C@H]1CCCN(Cc2cc3c(C4CC4)cn(-c4cccc(C(F)c5nncn5C)c4)c(=O)c3n2COCC[Si](C)(C)C)C1 ; C[C@H]1CCCN(Cc2cc3c(C4CC4)cn(-c4cccc(C(F)c5nncn5C)c4)c(=O)c3[nH]2)C1]

Step a: To a solution of (3-bromophenyl)-(4-methyl-1,2,4-triazol-3-yl)methanol (0.26 g, 1.0 mmol, 1 equiv., obtained according to example 159) in dichloromethane (10 mL) Deoxo-Fluor® (0.66 g, 3.0 mmol, 3 equiv.) was added dropwise at 0° C. The resulting mixture was stirred at 0° C. for 20 min, allowed to warm to room temperature and stirred 50° C. overnight. The resulting mixture was cooled to room temperature and quenched with water (20 mL). The crude product was extracted with chloroform/isopropanol mixture (2×20 mL, 3:1, v/v). The combined organic solution was dried over Na₂SO₄ and concentrated to dryness under reduced pressure. The product was purified by column chromatography (SiO₂, 0-10% MeOH in dichloromethane gradient) to furnish the desired product.

Steps b-c: The steps b-c were performed in a similar fashion to example 161. ¹H NMR (400 MHz, CDCl₃) δ 10.70 (s, 1H), 8.12 (s, 1H), 7.54 (t, J=7.7 Hz, 1H), 7.44 (s, 1H), 7.43-7.36 (m, 2H), 7.00 (d, J=46.0 Hz, 1H), 6.73 (d, J=1.2 Hz, 1H), 6.44 (s, 1H), 3.78 (s, 2H), 3.62 (s, 3H), 3.02-2.84 (m, 2H), 2.07 (td, J=11.1, 5.4 Hz, 1H), 1.95-1.82 (m, 1H), 1.82-1.61 (m, 4H), 0.92-0.80 (m, 6H), 0.68-0.59 (m, 2H). ESI MS [M+H]⁺ for C₂₇H₃₁FN₆O, calcd 475.3, found 475.2.

### Example 163: 4-cyclopropyl-6-[3-[1-fluoro-1-(4-methyl-1,2,4-triazol-3-yl)ethyl]phenyl]-2-[[(3S)-3-methylpiperidin-1-yl]methyl]-1H-pyrrolo[2,3-c]pyridin-7-one

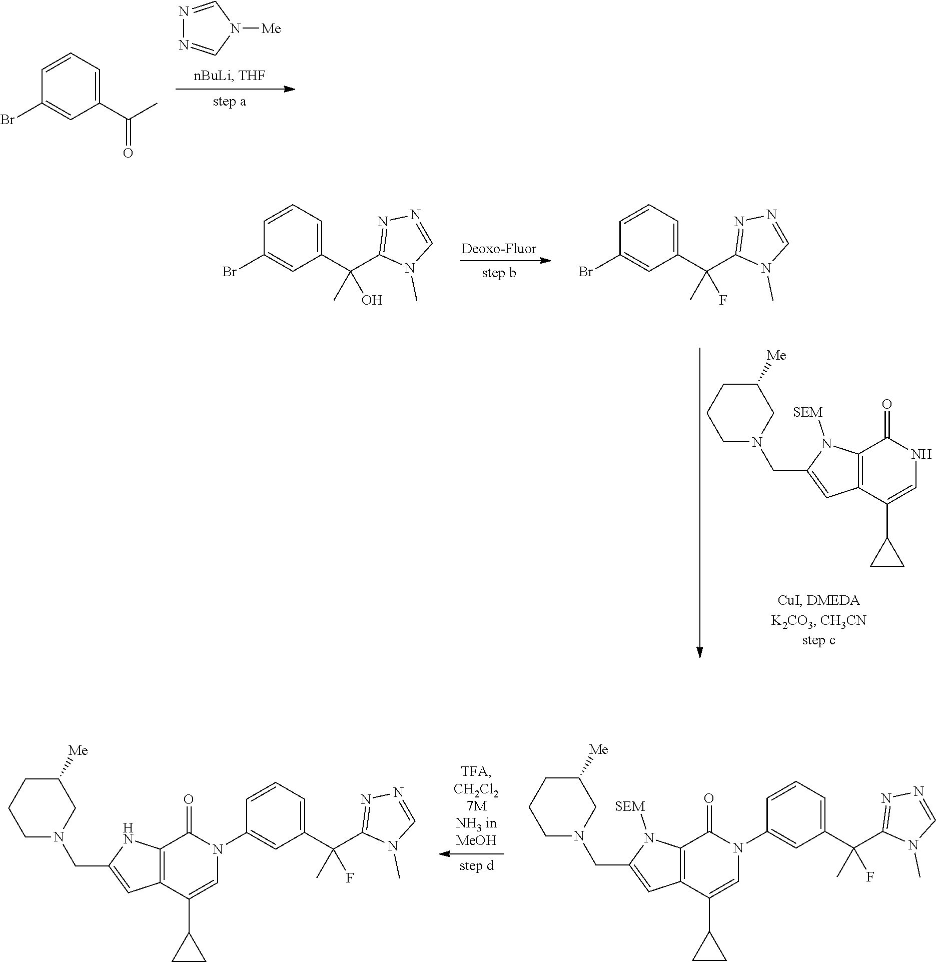

Step a: nBuLi (2.4 mL, 6.0 mmol, 1.2 equiv.) was added dropwise to the solution of 4-methyl-4H-1,2,4-triazole (0.5 g, 6.0 mmol, 1.2 equiv.) in DME (20 mL) at −40° C. The resulting mixture was stirred at −40° C. for 1 h, then 1-(3-bromophenyl)ethanone (1.0 g, 5.0 mmol, 1 equiv.) was added. The reaction mixture was stirred at −40° C. for 1 h, then warmed to 0° C. and quenched with aq. NH₄Cl (20 mL). The product was extracted with EtOAc (3×20 mL), combined organic extract was dried over Na₂SO₄ and concentrated to dryness under reduced pressure. Purification by column chromatography (SiO₂, 0-50% EtOAc in hexanes gradient) afforded the desired product.

Step b: To the solution of the product from step a (0.28 g, 1.0 mmol, 1 equiv.) in dichloromethane (10 mL), Deoxo-Fluor® (0.66 g, 3.0 mmol, 3 equiv.) was added dropwise at 0° C., and the reaction was stirred at this temperature for 1 h. The reaction was quenched by water (10 mL), and the product was extracted with dichloromethane (3×15 mL). The combined organic extract was dried over Na₂SO₄ and concentrated to dryness under reduced pressure. The crude residue was purified by column chromatography (SiO₂, 0-10% MeOH in dichloromethane gradient) to give the desired product.

Steps c-d: The steps c-d were performed in a similar fashion to example 161. ¹H NMR (400 MHz, CDCl₃) δ 10.46 (s, 1H), 8.09 (s, 1H), 7.49 (t, J=7.8 Hz, 1H), 7.41-7.33 (m, 2H), 7.28 (d, J=8.6 Hz, 1H), 6.71 (d, J=1.2 Hz, 1H), 6.42 (s, 1H), 5.30 (s, 1H), 3.76 (d, J=1.5 Hz, 2H), 3.48 (d, J=1.4 Hz, 3H), 2.93 (dd, J=24.0, 9.9 Hz, 2H), 2.28 (d, J=23.3 Hz, 3H), 2.07 (td, J=10.7, 7.2 Hz, 1H), 1.88 (dddd, J=11.7, 8.3, 5.3, 1.2 Hz, 1H), 1.80-1.72 (m, 2H), 1.68 (dd, J=7.7, 3.8 Hz, 2H), 0.94-0.80 (m, 5H), 0.64 (td, J=5.7, 4.1 Hz, 2H). ESI MS [M+H]⁺ for C₂₈H₃₃FN₆O, calcd 489.3, found 489.2.

### Example 164: 4-cyclopropyl-6-[3-[1-fluoro-1-(1-methylimidazol-2-yl)ethyl]phenyl]-2-[[(3S)-3-methylpiperidin-1-yl]methyl]-1-(2-trimethylsilylethoxymethyl)pyrrolo[2,3-c]pyridin-7-one

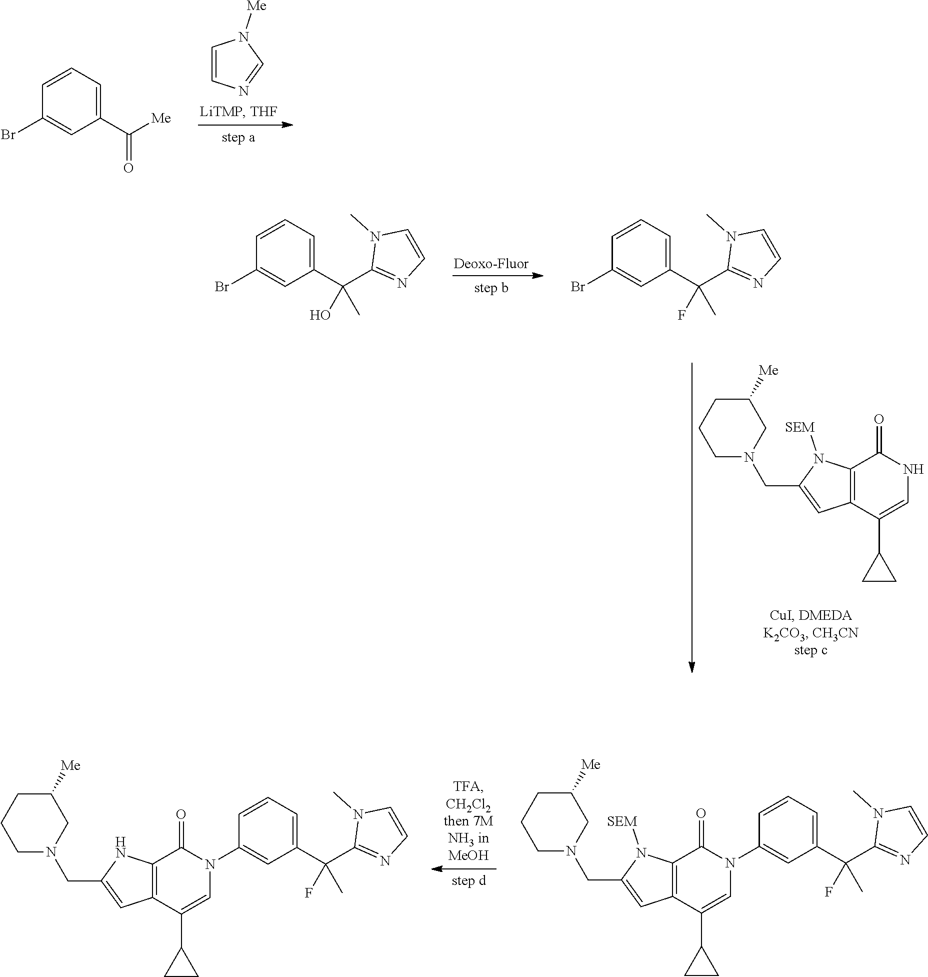

Step a: TMP (0.4 mL, 2.4 mmol, 1.2 equiv.) was dissolved in THE (10 mL) and n-BuLi (1 mL, 2.4 mmol, 1.2 equiv.) was added at −78° C. The reaction mixture was stirred for 30 minsfollowed by addition N-methyl imidazole (157 mg, 2.0 mmol, 1 equiv.). After 0.5 h at −78° C., 1-(3-bromophenyl)ethanone (0.4 g, 2.0 mmol, 1 equiv.) was added at −78° C. The mixture was stirred at this temperature for 2 h, then allowed to warm to 0° C. and quenched with aq. sat. NH₄Cl (10 mL). The product was extracted with EtOAc (3×20 mL). Combined organic phase was dried over Na₂SO₄ and concentrated to dryness under reduced pressure. The crude product was purified by column chromatography (SiO₂, 0-50% EtOAc in hexanes gradient).

Step b: To the solution of the product from step a (0.42 g, 1.5 mmol, 1 equiv.) in dichloromethane (15 mL) Deoxo-Fluor® (1.0 g, 4.5 mmol, 3 equiv.) was added dropwise at 0° C. The resulting mixture was stirred at 0° C. for 1 h. The reaction was quenched by water (10 mL), and the product was extracted with dichloromethane (3×15 mL). The combined organic extract was dried over Na₂SO₄ and concentrated to dryness under reduced pressure. The crude residue was purified by column chromatography (SiO₂, 0-100% EtOAc in hexanes gradient) to give the desired product.

Step c-d: The steps c-d were performed in a similar fashion to example 161. ¹H NMR (400 MHz, CDCl₃) δ 7.52-7.39 (m, 1H), 7.39-7.34 (m, 1H), 7.32 (t, J=2.0 Hz, 1H), 7.22 (d, J=7.9 Hz, 1H), 6.98 (dd, J=20.9, 1.2 Hz, 1H), 6.88-6.78 (m, 1H), 6.75-6.67 (m, 1H), 6.39 (d, J=6.0 Hz, 1H), 3.69 (d, J=12.6 Hz, 2H), 3.46-3.35 (m, 3H), 2.84 (dd, J=19.7, 9.8 Hz, 2H), 2.18 (d, J=22.9 Hz, 2H), 2.08-2.05 (m, 1H), 1.98 (d, J=12.6 Hz, 1H), 1.87 (qd, J=8.7, 8.0, 5.9 Hz, 1H), 1.67 (d, J=29.6 Hz, 6H), 0.91-0.76 (m, 6H), 0.64 (dd, J=5.7, 4.3 Hz, 2H). ESI MS [M+H]⁻ for C₂₉H₃₄FN₅O, calcd 488.3, found 488.2.

### Example 165: 4-cyclopropyl-2-[[(3S)-3-methylpiperidin-1-yl]methyl]-6-[3-[1-(4-methyl-1,2,4-triazol-3-yl)ethyl]phenyl]-1H-pyrrolo[2,3-c]pyridin-7-one

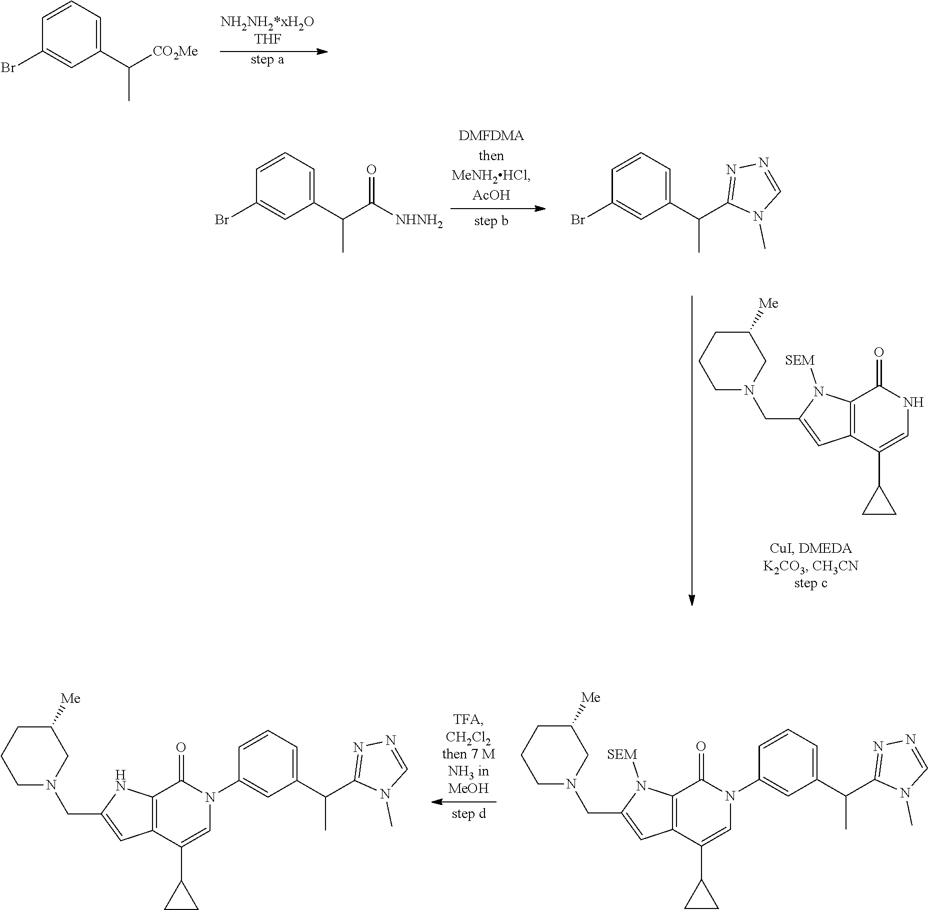 [SMILES: COC(=O)C(C)c1cccc(Br)c1 ; CC(C(=O)NN)c1cccc(Br)c1 ; CC(c1cccc(Br)c1)c1nncn1C ; C[C@H]1CCCN(Cc2cc3c(C4CC4)c[nH]c(=O)c3n2COCC[Si](C)(C)C)C1 ; CC(c1cccc(-n2cc(C3CC3)c3cc(CN4CCC[C@H](C)C4)[nH]c3c2=O)c1)c1nncn1C ; CC(c1cccc(-n2cc(C3CC3)c3cc(CN4CCC[C@H](C)C4)n(COCC[Si](C)(C)C)c3c2=O)c1)c1nncn1C]

Step a: Methyl 3-bromo-a-methylbenzeneacetate (1.0 g, 4.1 mmol, 1 equiv.) was dissolved in THF (20 mL), then hydrazine hydrate (10 mL, ˜35% solution) was added. The reaction was reflux for 48 h. The mixture was cooled to room temperature and partitioned between EtOAc (50 mL) and water (50 mL). The organic phase was separated, and the aqueous solution was additionally extracted with EtOAc (2×20 mL). Combined organic phase was dried over Na₂SO₄ and concentrated to dryness under reduced pressure. The crude product was used for the next step without purification.

Step b: The product from step a (0.33 g, 1.4 mmol) was dissolved in DMFDMA (5 mL), and the reaction was reflux at 80° C. for 1 h. The solvent was removed under reduced pressure. The crude product was dissolved in acetic acid (5 mL), methylamine hydrochloride (183 mg, 2.7 mmol) was added, and the reaction was stirred at 90° C. for 30 mins. Upon solvent removal under reduced pressure the crude product was purified by column chromatography (SiO₂, 0-60% EtOAc in hexanes gradient) to yield the desired product.

Step c-d: The steps c-d were performed in a similar fashion to example 161. ¹H NMR (400 MHz, CDCl₃) δ 10.94 (s, 1H), 8.06 (s, 1H), 7.43 (t, J=7.7 Hz, 1H), 7.29 (d, J=1.7 Hz, 1H), 7.26-7.16 (m, 2H), 6.72 (d, J=1.2 Hz, 1H), 6.45 (s, 1H), 4.23 (q, J=7.1 Hz, 1H), 3.86 (s, 2H), 3.42 (s, 3H), 3.00 (dd, J=27.9, 9.9 Hz, 5H), 2.22-2.07 (m, 1H), 1.86 (d, J=7.1 Hz, 5H), 1.74 (d, J=20.4 Hz, 2H), 0.86 (dq, J=5.9, 4.2 Hz, 5H), 0.63 (td, J=5.4, 3.6 Hz, 2H). ESI MS [M+H]⁻ for C₂₈H₃₄N₆O, calcd 471.3, found 471.2.

### Biological Assay Examples

The affinity with which compounds of the present dislcosure bind to Cbl-b was assessed using probe displacement homologous time resolved fluorescence (HTRF) assays. The assays used a BODIPY™ conjugated probe (Example 54 from WO 2020264398) and biotinylated Cbl-b. The assays were performed in assay buffer consisting of 20 mM Hepes, 150 mM NaCl, 0.01% Triton X-100, 0.5 mM TCEP, 0.01% BSA. On the day of the assay, a 20 point, 1:2 master serial dilution of each compound was prepared in DMSO to span a final concentration range of 10 M to 0 nM. Two hundred nanoliters of diluted compound was added to each well of a 384-well plate. Fifteen microliters of biotinylated-Cbl-b resuspended in assay buffer were added to each well and the plate incubated for 60 minutes at room temperature prior to addition of 5 μL of BODIPY™ probe in assay buffer. Final assay conditions included 0.4 nM of Cbl-b and 150 nM of BODIPY™ probe. After a further 15 minutes of incubation at room temperature, 5 μl of Streptavidin-Terbium cryptate reagent at 5-fold final concentration was added to each well of the 384-well plate. Binding of a probe to Cbl-b results in an increase of HTRF signal. HTRF signal was measured by Envision plate reader, while competition of a compound of the present disclosure with probe results in a decrease of signal. Percentage maximum activity in each test well was calculated based on DMSO (maximum activity, 0% displacement) and no protein control wells (baseline activity, 100% displacement). Binding affinity was determined from a dose response curve fitted using a standard four parameter fit equation. See Table 3 for data for compounds (Cbl-b Binding (IC₅₀)).

Certain compounds were also evaluated in an IL-2 secretion assay. On the day of the assay, a 16 point, 1:2 master serial dilution of each compound was prepared in Opti-MEM to span a final concentration range of 10 μM to 300 pM. Assays were set up in CORNING® tissue culture-treated 384-well microplates containing 60 nL of each dilution. Jurkat cells grown in RPMI-1640 supplemented with 10% FBS, 1% Glutamax, and 1% Pen/Strep were collected, resuspended in Opti-MEM. 50,000 cells/well and added to the compound plates. After a short spin (1200 rpm for 1 min), the plates were incubated at 37° C. for 1 hour. The cells were activated by adding 15 μL of IMMUNOCULT™ Human CD3/CD28 T Cell Activator (STEMCELL Technologies) diluted in Opti-MEM. After 24 h of incubation at 37° C., aliquots of culture supernatants were transferred to OptiPlate-384 (PerkinElmer) microplates. The level of IL-2 secretion in the supernatants was then determined using the IL-2 (human) AlphaLISA Detection Kit (PerkinElmer) according to the manufacturer's recommendations. The AiphaLISA signal was measured using an EnVision plate reader (PerkinElmer). EC₅₀ values were determined by fitting the data to a standard 4-parameter logistic equation. See Table 3 for data for select compounds (IL-2 Secretion (EC₅₀)).

TABLE 3
Potency of select compounds.

| Example # | Cbl-b Binding (IC₅₀) | IL-2 Secretion (EC₅₀) |
|---|---|---|
| Example 1 | +++ | |
| Example 2 | ++ | |
| Example 3 | +++ | +++ |
| Example 4 | ++ | + |
| Example 5 | − | |
| Example 6 | − | |
| Example 7 | +++ | + |
| Example 8 | + | + |
| Example 9 | ++ | + |
| Example 10 | ++ | |
| Example 11 | ++ | |
| Example 12 | ++ | + |
| Example 13 | ++ | |
| Example 14 | +++ | ++ |
| Example 15 | ++ | |
| Example 16 | ++ | |
| Example 17 | ++ | |
| Example 18 | ++ | |
| Example 19 | + | |
| Example 20 | + | |
| Example 21 | +++ | |
| Example 22 | +++ | |
| Example 23 | ++ | |
| Example 24 | ++ | |
| Example 25 | ++ | |
| Example 26 | ++ | |
| Example 27 | ++ | |
| Example 28 | ++ | |
| Example 29 | ++ | |
| Example 30 | + | |
| Example 31 | + | |
| Example 32 | + | |
| Example 33 | ++ | |
| Example 34 | +++ | + |
| Example 35 | +++ | |
| Example 36 | +++ | ++ |
| Example 37 | +++ | ++ |
| Example 38 | +++ | + |
| Example 39 | +++ | +++ |
| Example 40 | +++ | ++ |
| Example 41 | ++ | + |
| Example 42 | +++ | +++ |
| Example 43 | +++ | +++ |
| Example 44 | +++ | |
| Example 45 | +++ | +++ |
| Example 46 | +++ | ++ |
| Example 47 | +++ | +++ |
| Example 48 | +++ | |
| Example 49 | +++ | +++ |
| Example 50 | +++ | +++ |
| Example 51 | +++ | ++ |
| Example 52 | +++ | +++ |
| Example 53 | +++ | |
| Example 54 | +++ | ++ |
| Example 55 | +++ | +++ |
| Example 56 | +++ | ++ |
| Example 57 | +++ | ++ |
| Example 58 | +++ | ++ |
| Example 59 | +++ | − |
| Example 60 | +++ | + |
| Example 61 | ++ | + |
| Example 62 | ++ | − |
| Example 63 | +++ | +++ |
| Example 64 | +++ | ++ |
| Example 65 | +++ | + |
| Example 66 | ++ | + |
| Example 67 | ++ | + |
| Example 68 | +++ | ++ |
| Example 69 | ++ | + |
| Example 70 | +++ | ++ |
| Example 71 | +++ | ++ |
| Example 72 | ++ | + |
| Example 73 | +++ | ++ |
| Example 74 | +++ | +++ |
| Example 75 | +++ | +++ |
| Example 76 | +++ | ++ |
| Example 77 | +++ | +++ |
| Example 78 | +++ | +++ |
| Example 79 | ++ | ++ |
| Example 80 | +++ | |
| Example 81 | +++ | |
| Example 82 | +++ | + |
| Example 83 | +++ | ++ |
| Example 84 | +++ | + |
| Example 85 | +++ | ++ |
| Example 86 | ++ | + |
| Example 87 | ++ | − |
| Example 88 | +++ | +++ |
| Example 89 | +++ | ++ |
| Example 90 | − | − |
| Example 91 | ++ | − |
| Example 92 | ++ | − |
| Example 93 | ++ | + |
| Example 94 | +++ | +++ |
| Example 95 | +++ | +++ |
| Example 96 | +++ | +++ |
| Example 97 | +++ | ++ |
| Example 98 | +++ | +++ |
| Example 99 | ++ | + |
| Example 100 | +++ | ++ |
| Example 101 | +++ | +++ |
| Example 102 | +++ | +++ |
| Example 103 | +++ | ++ |
| Example 104 | +++ | +++ |
| Example 105 | +++ | +++ |
| Example 106 | +++ | +++ |
| Example 107 | +++ | +++ |
| Example 108 | ++ | − |
| Example 109 | +++ | +++ |
| Example 110 | +++ | ++ |
| Example 111 | +++ | +++ |
| Example 112 | +++ | +++ |
| Example 113 | +++ | +++ |
| Example 114 | ++ | − |
| Example 115 | + | − |
| Example 116 | + | |
| Example 117 | ++ | − |
| Example 118 | ++ | − |
| Example 119 | +++ | +++ |
| Example 120 | +++ | ++ |
| Example 121 | ++ | ++ |
| Example 122 | ++ | ++ |
| Example 123 | ++ | ++ |
| Example 124 | ++ | ++ |
| Example 125 | + | + |
| Example 126 | +++ | +++ |
| Example 127 | ++ | ++ |
| Example 128 | +++ | ++ |
| Example 129 | +++ | +++ |
| Example 130 | +++ | +++ |
| Example 131 | +++ | − |
| Example 132 | +++ | + |
| Example 133 | +++ | − |
| Example 134 | +++ | +++ |
| Example 135 | +++ | +++ |
| Example 136 | +++ | |
| Example 137 | +++ | + |
| Example 138 | +++ | |
| Example 139 | +++ | |
| Example 140 | +++ | ++ |
| Example 141 | + | − |
| Example 142 | + | − |
| Example 143 | +++ | ++ |
| Example 144 | +++ | ++ |
| Example 145 | +++ | + |
| Example 146 | +++ | ++ |
| Example 147 | +++ | + |
| Example 148 | +++ | +++ |
| Example 149 | +++ | +++ |
| Example 150 | +++ | +++ |
| Example 151 | +++ | +++ |
| Example 152 | +++ | + |
| Example 153 | +++ | |
| Example 154 | +++ | ++ |
| Example 155 | +++ | ++ |
| Example 156 | +++ | ++ |
| Example 157 | +++ | |
| Example 158 | +++ | ++ |
| Example 159 | ++ | ++ |
| Example 160 | ++ | ++ |
| Example 161 | +++ | ++ |
| Example 162 | ++ | ++ |
| Example 163 | +++ | +++ |
| Example 164 | + | + |
| Example 165 | +++ | +++ |

Less than 100 nM (+++),
100 nM to 1 μM (++),
greater than 1 μM to 5 μM (+),
>5 μM (−),
blank = not determined

Particular embodiments of this disclosure are described herein, including the best mode known to the inventors for carrying out the disclosure. Upon reading the foregoing, description, variations of the disclosed embodiments may become apparent to individuals working in the art, and it is expected that those skilled artisans may employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference for the purpose described herein.

1. A compound having a structure according to Formula II: 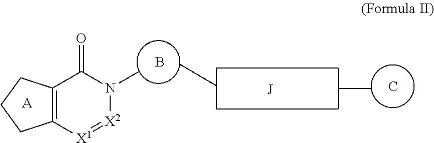
or a pharmaceutically acceptable salt thereof;
wherein:
ring A is selected from the group consisting of: 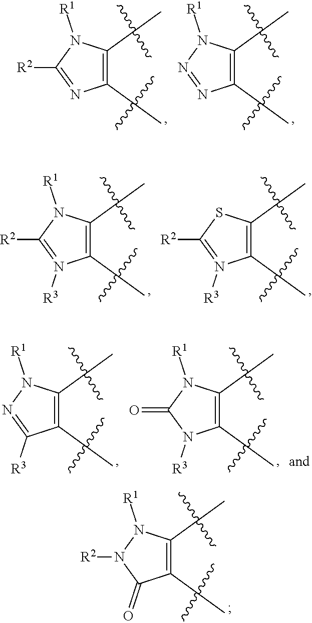
R¹, when present, is —H or C₁-C₃ alkyl;
R², when present, is H; —CN; —NR^{2a}R^{2b}, wherein R^{2a} and R^{2b} are independently selected from H and C₁-C₆ alkyl; —S(O)₂(C₁-C₃ alkyl); —S(O)(NR^{2c})(C₁-C₃ alkyl); —NR^{2c}—S(O)₂(C₁-C₃ alkyl); —S(O)₂—N(R^{2c})₂; 5- to 6-membered heteroaryl containing 1-3 ring heteroatoms independently selected from N, S, and O, and the 5- or 6-membered heteroaryl is substituted with 0-3 C₁-C₃ alkyl; 5- or 6-membered heterocycloalkyl ring having 1-2 ring heteroatoms independently selected from N, S, and O, and the 5- or 6-membered heterocycloalkyl ring is substituted with 0-3 C₁-C₃ alkyl; unsubstituted C₁-C₃ alkyl; or C₁-C₃ alkyl substituted with (i) —OR^{2c}, (ii) —C(O)NR^{a}R^{b}, (iii) —NR^{a}R^{b}, (iv) a 4- to 8-membered heterocycloalkyl ring having 1-2 ring heteroatoms independently selected from N, S, and O, and the 4- to 8-membered heterocycloalkyl ring is substituted with 0-3 substituents independently selected from halo, —OH, C₁-C₃ alkyl, C₁-C₃ haloalkyl, and C₁-C₃ hydroxyalkyl, or (v) 5- to 6-membered heteroaryl containing 1-3 ring heteroatoms independently selected from N, S, and O; wherein R²⁰ is H or C₁-C₃ alkyl; and R^{a} and R^{b} are independently H, C₁-C₃ alkyl, phenyl, —(C₁-C₃ alkylene)-O—(C₁-C₃ alkyl), C₃-C₆ cycloalkyl, —(C₁-C₃ alkylene)-(C₃-C₆ cycloalkyl), S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-0-(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2d}, when present, is independently halo, —OH, C₁-C₃ alkyl, C₁-C₃ hydroxyalkyl, or C₁-C₃ haloalkyl;
R³, when present, is H; —CN; C₁-C₃ alkyl; C₁-C₃ alkylene-NR^{c}R^{d}; C₁-C₃ haloalkyl; C₁-C₃ hydroxyalkyl; C₃-C₆ cycloalkyl; —S(O)₂(C₁-C₃ alkyl); —COR^{c}; —COOR^{c}; —CONR^{c}R^{d}; or 5-membered heteroaryl containing 1-3 ring heteroatoms independently selected from N, O, and S, and the 5-membered heteroaryl is substituted with 0-3 substituents independently selected from halo, OH, CN, C₁-C₃ alkyl, C₁-C₃ haloalkyl, C₁-C₃ hydroxyalkyl, —C(O)NR^{c}R^{d}, —NR^{c}R^{d}, —COR^{c}, —COOR^{c}, —S(O)₂(C₁-C₃ alkyl), —NR^{c}—S(O)₂(C₁-C₃ alkyl), and —S(O)₂—NR^{c}R^{d}; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is substituted with 0-3 C₁-C₃ alkyl;
X¹ is CR⁴ or N;
X² is CR⁵ or N;
R⁴, when present, is H; —CN; halogen; C₁-C₃ alkyl; C₁-C₃ haloalkyl; C₁-C₃ hydroxyalkyl; C₂-C₃ alkenyl; C₃-C₄ cycloalkyl; —S(O)₂(C₁-C₃ alkyl); —C(O)OH; or 5- or 6-membered heteroaryl having 1 to 4 ring heteroatoms independently selected from N, S, and O, and the heteroaryl is substituted with 0-3 C₁-C₃ alkyl;
R⁵ is H or C₁-C₃ alkyl;
ring B is phenylene, or 5- to 6-membered heteroarylene having 1-3 ring heteroatoms independently selected from N, S, and O;
J is —(CR^{g}R^{h})ₙ—Y¹— or —Y¹—(CR^{g}R^{h})ₙ—; wherein n is 1 or 2; Y¹ is absent, CH₂, CHF, CF₂, O, S, S(O), or S(O)₂; and each R^{g} and each R^{h} is independently H, halo, —OH, C₁-C₃ alkyl, C₁-C₃ haloalkyl, C₁-C₃ alkoxy or C₃-C₄ cycloalkyl; or R^{g} and R^{h} together with the carbon atom to which they are attached form a 3- to 4-membered heterocycle ring having 1 ring heteroatom selected from N, O, and S, a C₃-C₄ cycloalkyl ring, or a C₆-C₈ spirocyclic ring, where each cycloalkyl ring or spirocyclic ring is substituted with 0-3 R³; and each R³ is independently selected from —CN, —OH, halogen, C₁-C₃ alkyl, C₁-C₃ alkoxy, and C₁-C₃ haloalkoxy; and
ring C is 5- to 6-membered heteroaryl containing 1-3 ring heteroatoms independently selected from N, S, and O, and the 5- to 6-membered heteroaryl is substituted with 0-3 substituents independently selected from C₁-C₃ alkyl, C₁-C₃ haloalkyl, and C₃-C₄ cycloalkyl.

2. (canceled)

3. The compound of claim 1, or a pharmaceutically acceptable salt thereof, wherein R², when present, is H; 5- to 6-membered heteroaryl containing 1-3 ring heteroatoms independently selected from N, S, and O, and the 5- or 6-membered heteroaryl is optionally substituted with 1-3 substituents independently selected from C₁-C₃ alkyl; 5- or 6-membered heterocycloalkyl ring optionally having 1-2 ring heteroatoms independently selected from N, S, and O, and the 5- or 6-membered heterocycloalkyl ring is optionally substituted with 1-3 substituents independently selected from C₁-C₃ alkyl; or C₁-C₃ alkyl optionally substituted with —OH, —NR^{a}R^{b}, or a 4- or 8-membered heterocycloalkyl ring optionally having 1-2 ring heteroatoms independently selected from N, S, and O, wherein the 4- or 8-membered heterocycloalkyl ring is optionally substituted with 1-3 substituents independently selected from —OH, C₁-C₃ alkyl, halo, C₁-C₃ haloalkyl, and C₁-C₃ hydroxyalkyl; and R^{a} and R^{b} are independently H, C₁-C₃ alkyl, phenyl, —(C₁-C₃ alkylene)-O—(C₁-C₃ alkyl), C₃—C₆ cycloalkyl, —(C₁-C₃ alkylene)-(C₃-C₆ cycloalkyl), S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-O—(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2d}, when present, is independently halo, —OH, C₁-C₃ alkyl, C₁-C₃ hydroxyalkyl, or C₁-C₃ haloalkyl.

4. The compound of claim 1, or a pharmaceutically acceptable salt thereof, wherein R², when present, is H, or C₁-C₃ alkyl substituted with —OH, —NR^{2a}R^{2b}, or 4- to 8-membered heterocycloalkyl ring having 1-2 ring heteroatoms independently selected from N, and O; wherein R^{2a} and R^{2b} are independently selected from H and C₁-C₆ alkyl; and the 4- to 8-membered heterocycloalkyl ring is substituted with 0-3 substituents independently selected from halo, and C₁-C₃ alkyl.

5-6. (canceled)

7. The compound of claim 1, or a pharmaceutically acceptable salt thereof, wherein R², when present, is:
C₁-C₃ alkyl optionally substituted with a 4- or 8-membered heterocycloalkyl ring optionally having 1-2 ring heteroatoms independently selected from N, S, and O, wherein the 5- or 6-membered heterocycloalkyl ring is optionally substituted with 1-3 substituents independently selected from —OH, C₁-C₃ alkyl, halo, C₁-C₃ haloalkyl, and C₁-C₃ hydroxyalkyl, or
C₁-C₃ alkyl optionally substituted with —OH or —NR^{a}R^{b}; and R^{a} and R^{b} are each independently H, C₁-C₃ alkyl, phenyl, —(C₁-C₃ alkylene)-O—(C₁-C₃ alkyl), C₃-C₆ cycloalkyl, —(C₁-C₃ alkylene)-(C₃-C₆ cycloalkyl), S(O)₂(C₁-C₃ alkyl), or 4- to 8-membered heterocycloalkyl having 1-3 ring heteroatom or heteroatom groups independently selected from N, O, S, and S(O)₂, wherein said phenyl, —(C₁-C₃ alkylene)-O—(C₁-C₃ alkyl), C₃-C₆ cycloalkyl, —(C₁-C₃ alkylene)-(C₃-C₆ cycloalkyl), and 4- to 8-membered heterocycloalkyl are substituted with 0-3 R^{2d}; wherein each R^{2d}, when present, is independently halo, —OH, C₁-C₃ alkyl, C₁-C₃ hydroxyalkyl, or C₁-C₃ haloalkyl.

8-10. (canceled)

11. The compound of claim 1, or a pharmaceutically acceptable salt thereof, wherein R², when present, is —H,

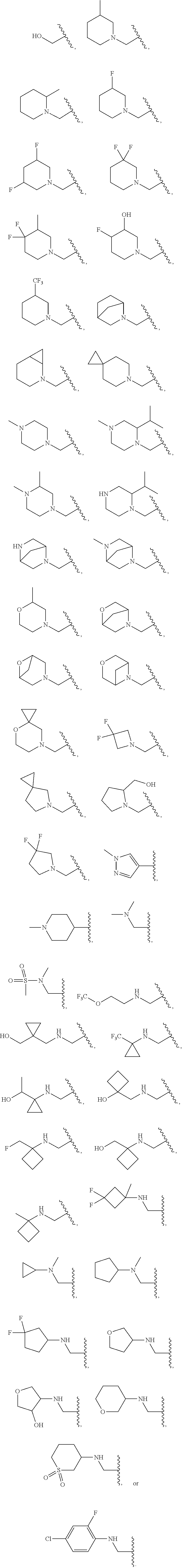

12. (canceled)

13. The compound of claim 1, or a pharmaceutically acceptable salt thereof, wherein R³, when present, is H; —CN; C₁-C₃ alkyl; C₁-C₃ alkylene-NR^{c}R^{d}; C₁-C₃ haloalkyl; C₁-C₃ hydroxyalkyl; C₃-C₆ cycloalkyl; —S(O)₂(C₁-C₃ alkyl); —COR^{c}; —COOR^{c}; or 5-membered heteroaryl having 1-3 ring nitrogen atoms, and the heteroaryl ring is unsubstituted or substituted with C₁-C₃ alkyl; wherein R^{c} and R^{d} are independently H or C₁-C₃ alkyl, or R^{c} and R^{d} together with the nitrogen atom to which they are attached form a 5- or 6-membered heterocycloalkyl ring optionally having 1 additional ring heteroatom selected from N, S, and O, and the 5- or 6-membered heterocycloalkyl ring is optionally substituted with 1-3 substituents independently selected from C₁-C₃ alkyl.

14. The compound of claim 1, or a pharmaceutically acceptable salt thereof, wherein R³, when present, is H, —CN, —CH₃, —CF₃, 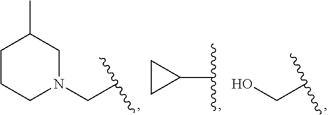
—CO₂H, —COCH₃, —CH(OH)CH₃, —SO₂CH₃,

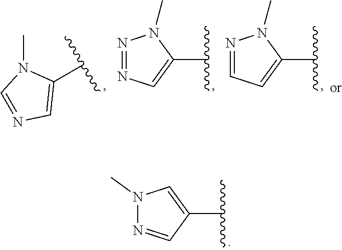

15. (canceled)

16. The compound of claim 1, or a pharmaceutically acceptable salt thereof, wherein R³, when present is H; and/or R¹, when present, is H.

17. (canceled)

18. The compound of claim 1, or a pharmaceutically acceptable salt thereof, wherein ring A is

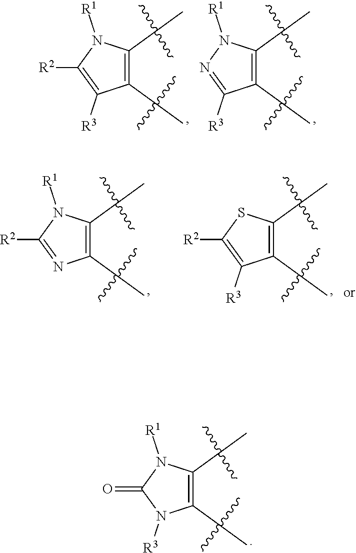

19-25. (canceled)

26. The compound of claim 1, or a pharmaceutically acceptable salt thereof, wherein X¹ is CR⁴; and R⁴ is C₃-C₄ cycloalkyl, H, CN, C₁-C₃ alkyl, C₁-C₃ haloalkyl, C₁-C₃ hydroxyalkyl, —S(O)₂(C₁-C₃ alkyl), or —C(O)OH.

27-28. (canceled)

29. The compound of claim 26, or a pharmaceutically acceptable salt thereof, wherein R⁴ is H, CN, methyl, CF₃, cyclopropyl, —S(O)₂CH₃, —C(O)OH, or —CH₂OH.

30. (canceled)

31. The compound of claim 1, or a pharmaceutically acceptable salt thereof, wherein X² is CR⁵; and R⁵ is H.

32. (canceled)

33. The compound of claim 1, or a pharmaceutically acceptable salt thereof, wherein ring B is phenylene or pyridylene.

34. (canceled)

35. The compound of claim 1, or a pharmaceutically acceptable salt thereof, wherein J is —(CR^{g}R^{h})—Y¹—; wherein Y¹ is absent, CH₂, or S; and each R^{g} and each R^{h} is independently H, halo, —OH, C₁-C₃ alkyl, C₁-C₃ haloalkyl, C₁-C₃ alkoxy, or C₃-C₄ cycloalkyl; or R^{g} and R^{h} together with the carbon atom to which they are attached form a 3- to 4-membered heterocycle ring having 1 ring oxygen atom, C₃-C₄ cycloalkyl ring, or a C₆-C₈ spirocyclic ring, where each cycloalkyl ring or spirocyclic ring is optionally substituted with 1-3 R^{j}; and each R^{j} is independently —OH, halogen, C₁-C₃ alkyl, C₁-C₃ alkoxy, and C₁-C₃ haloalkoxy.

36-37. (canceled)

38. The compound of claim 35, or a pharmaceutically acceptable salt thereof, wherein J is —(CR^{g}R^{h})—, and R^{g} and R^{h} together with the carbon atom to which they are attached form a C₃-C₄ cycloalkyl ring or a C₆-C₈ spirocyclic ring, where each cycloalkyl ring or spirocyclic ring is optionally substituted with 1-3 R³; and each R³ is independently selected from —OH, halogen, C₁-C₃ alkyl, C₁-C₃ alkoxy, and C₁-C₃ haloalkoxy.

39. (canceled)

40. The compound of claim 1, or a pharmaceutically acceptable salt thereof, wherein J is

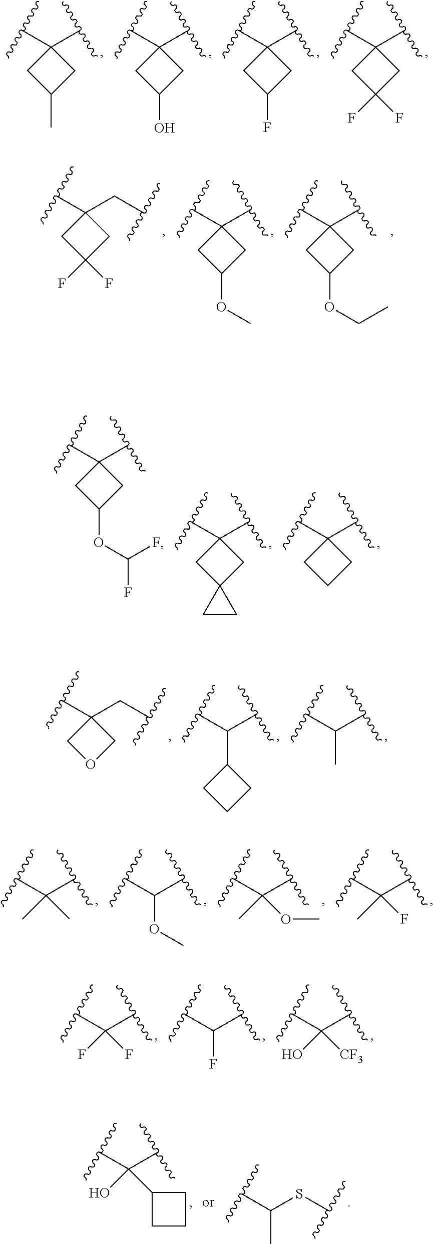

41-42. (canceled)

43. The compound of claim 1, or a pharmaceutically acceptable salt thereof, wherein ring C is a 5-membered heteroaryl containing 1-3 ring heteroatoms independently selected from O and N, and the 5-membered heteroaryl is substituted with 0-3 substituents independently selected from C₁-C₃ alkyl, C₁-C₃ haloalkyl, and C₃-C₆ cycloalkyl.

44. The compound of claim 43, or a pharmaceutically acceptable salt thereof, wherein ring C is imidazolyl, pyrazolyl, oxazolyl, oxadiazolyl, or triazolyl, each of which is substituted with 0-3 substituents independently selected from C₁-C₃ alkyl, C₁-C₃ haloalkyl, and C₃-C₆ cycloalkyl.

45. (canceled)

46. The compound of claim 1 selected from the group consisting of: 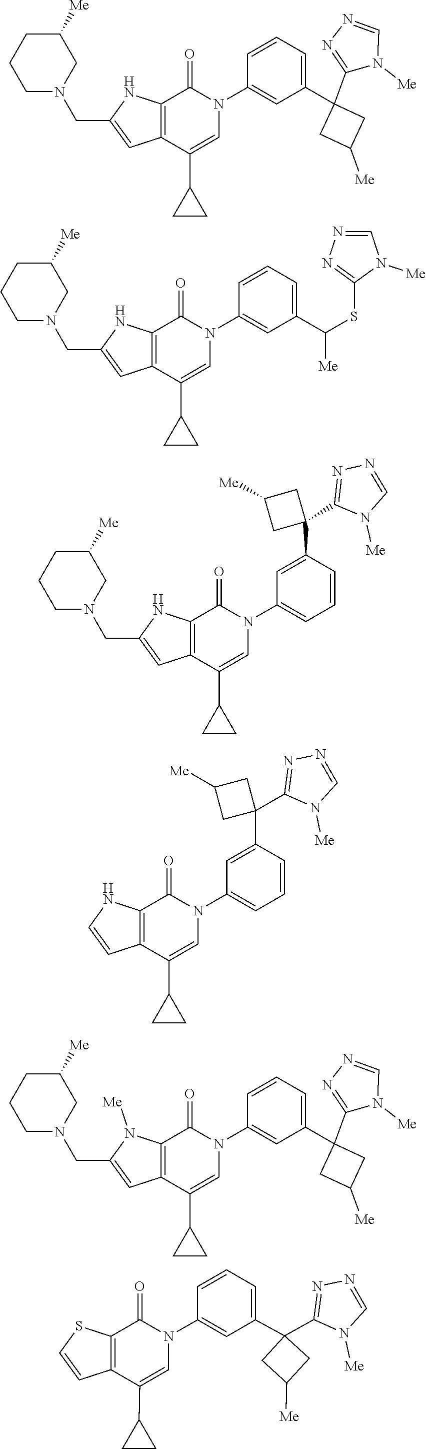 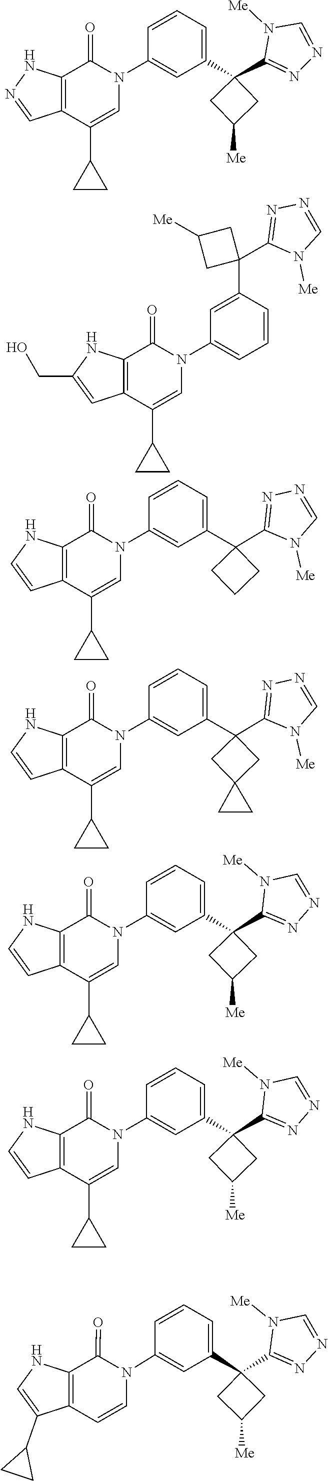 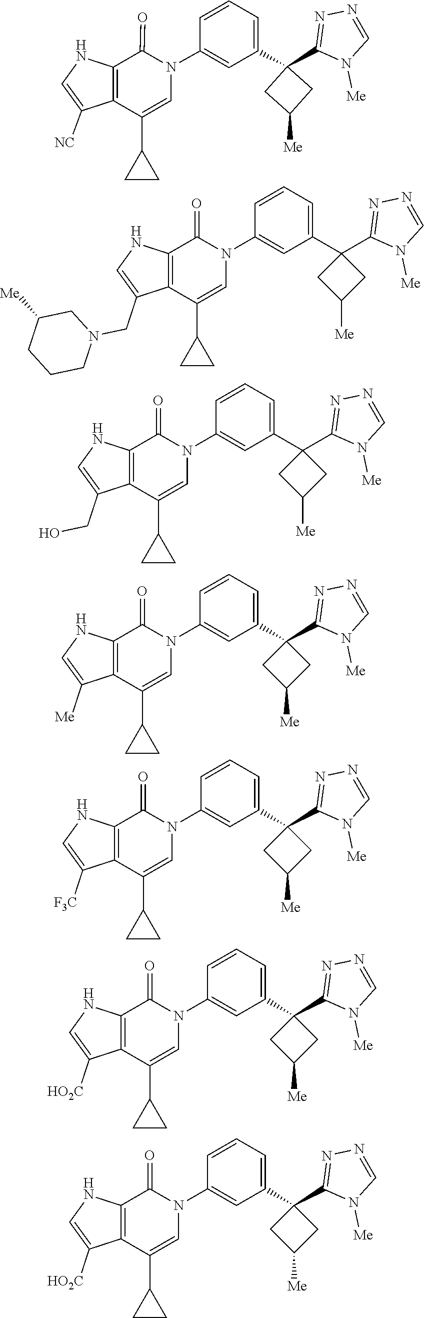 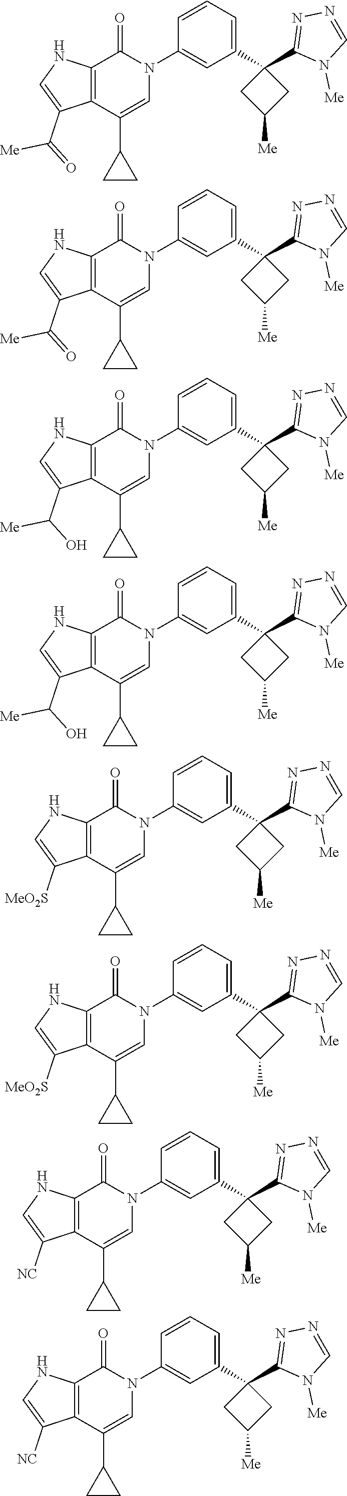 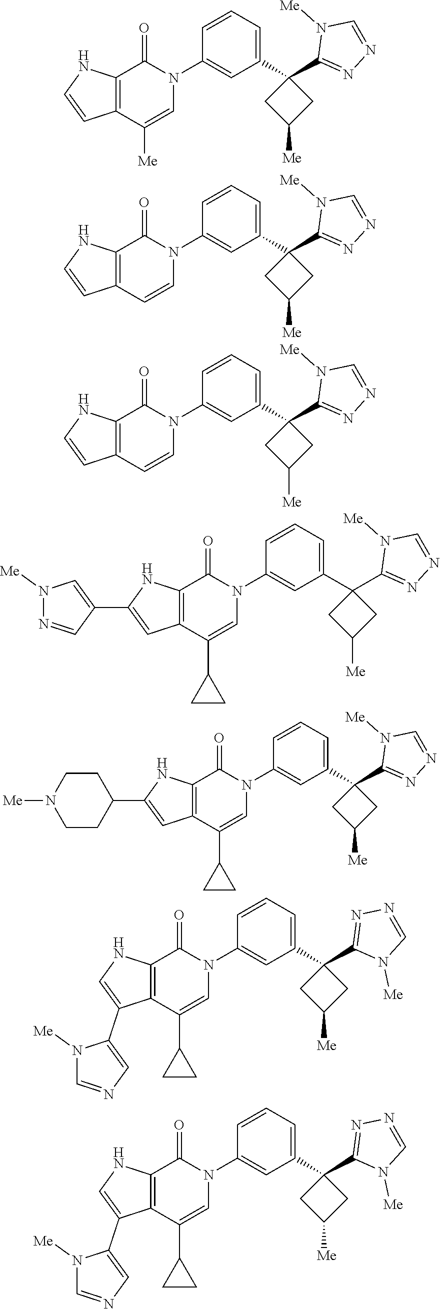 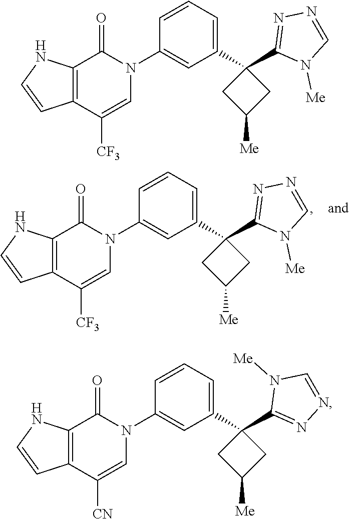
or a pharmaceutically acceptable salt thereof.

47. The compound of claim 1 selected from the group consisting of: 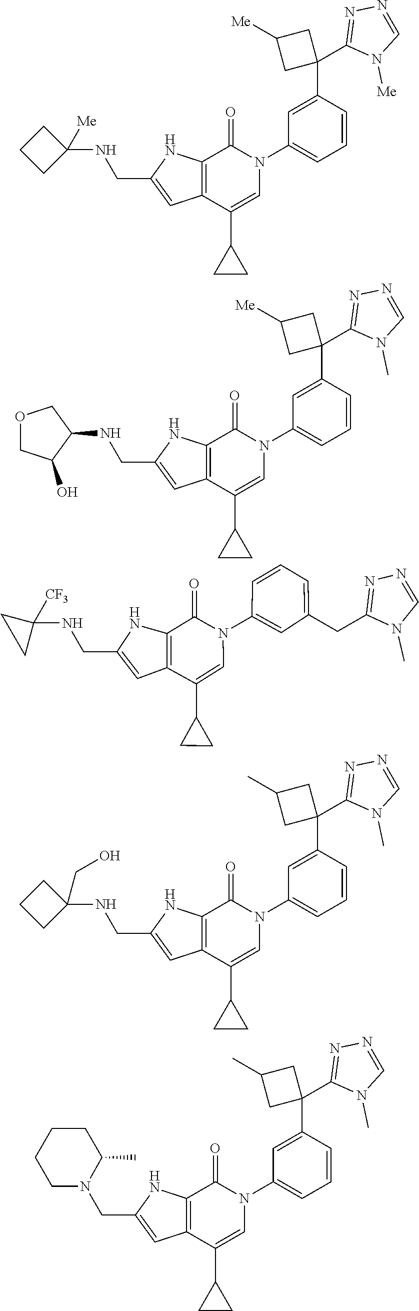 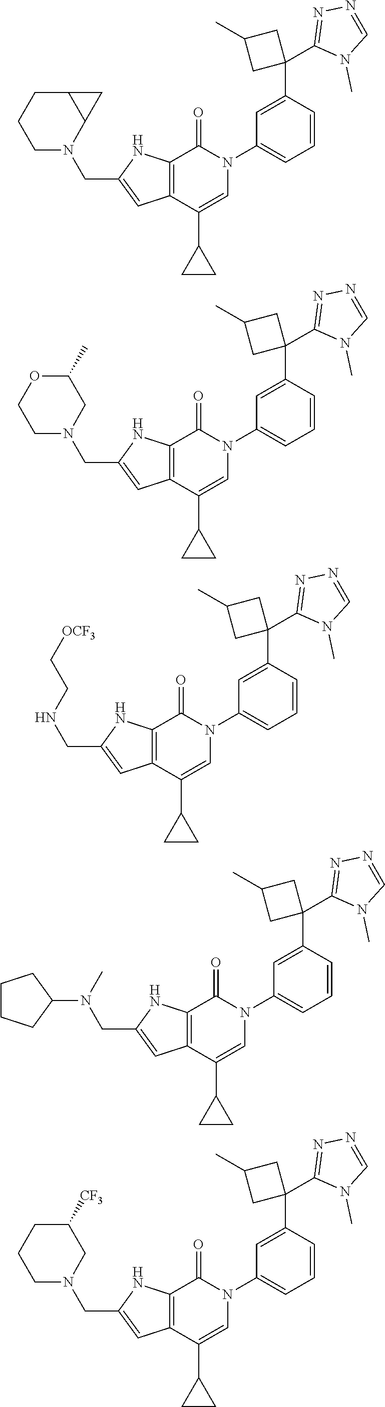 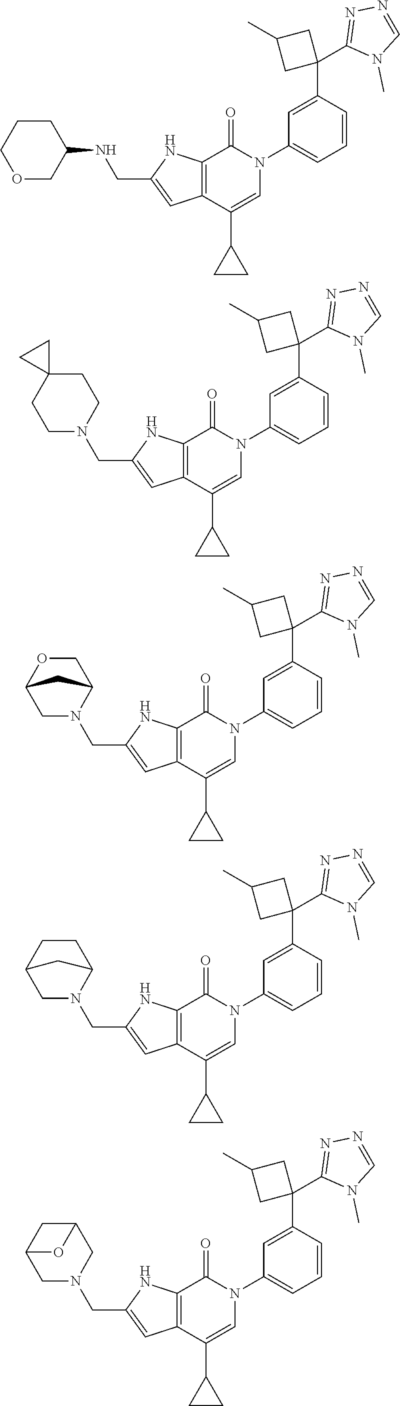 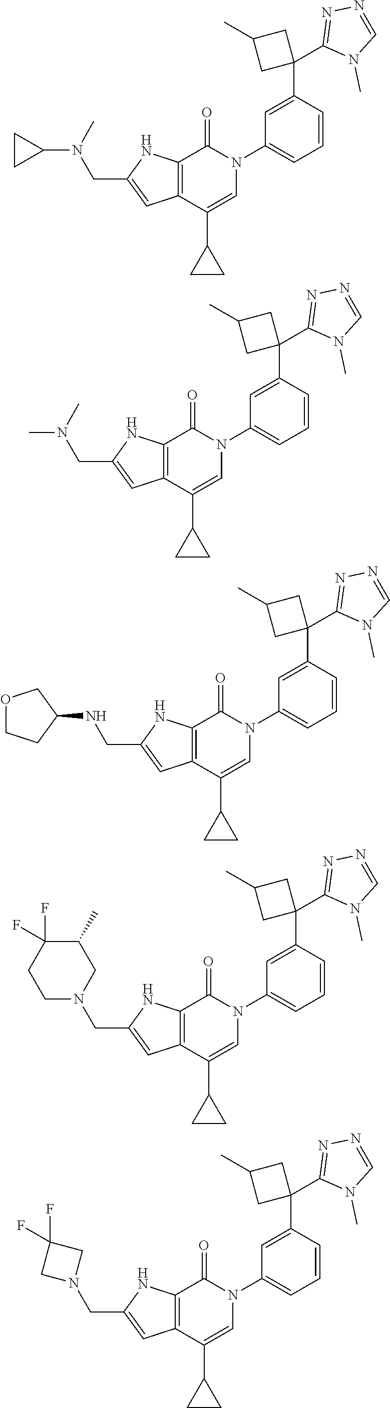 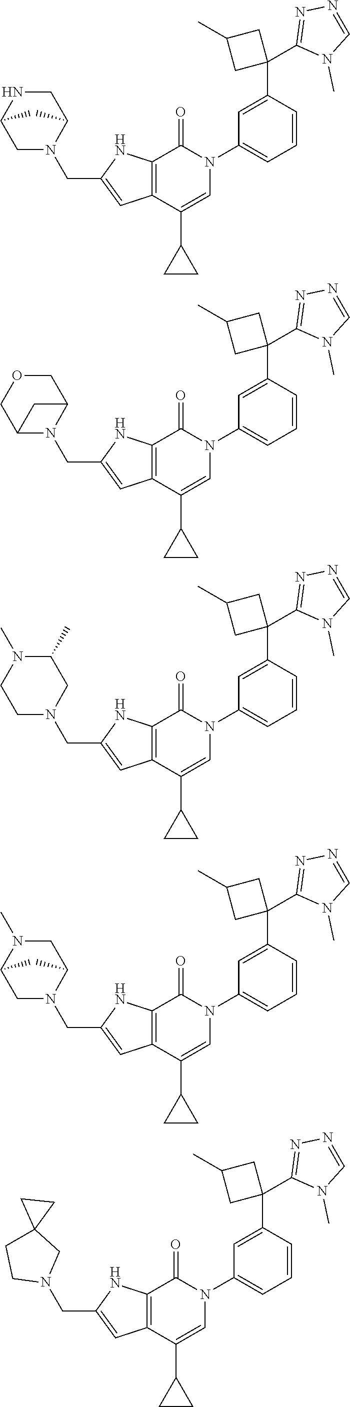 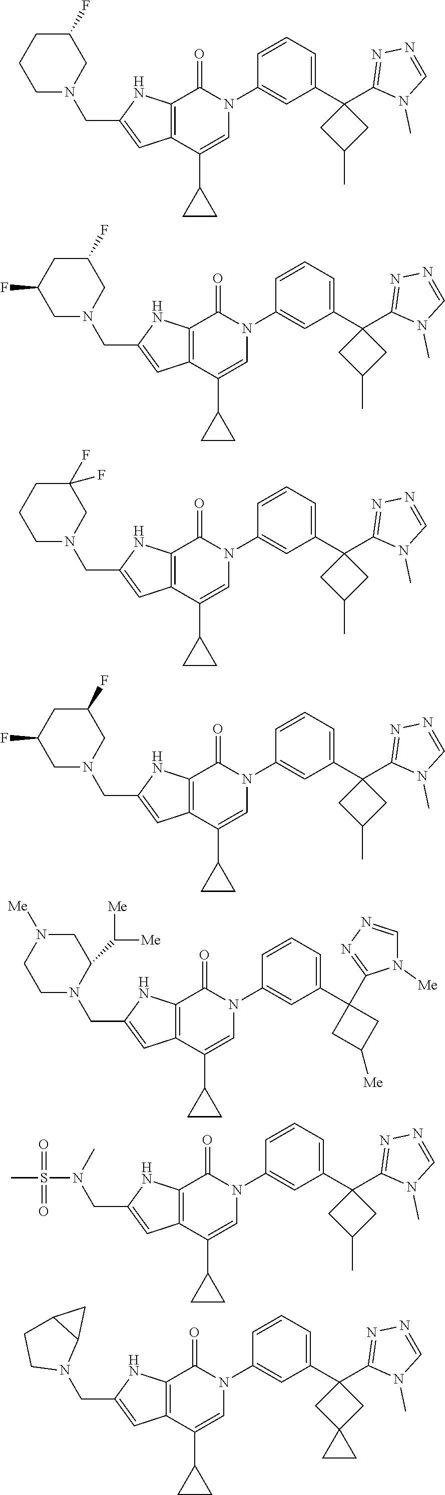 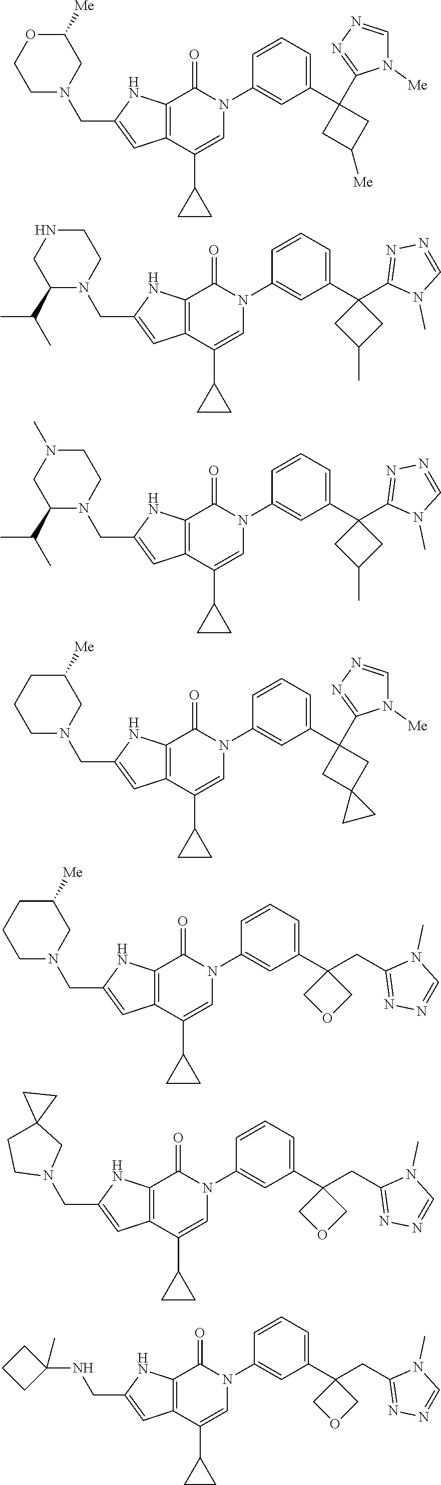 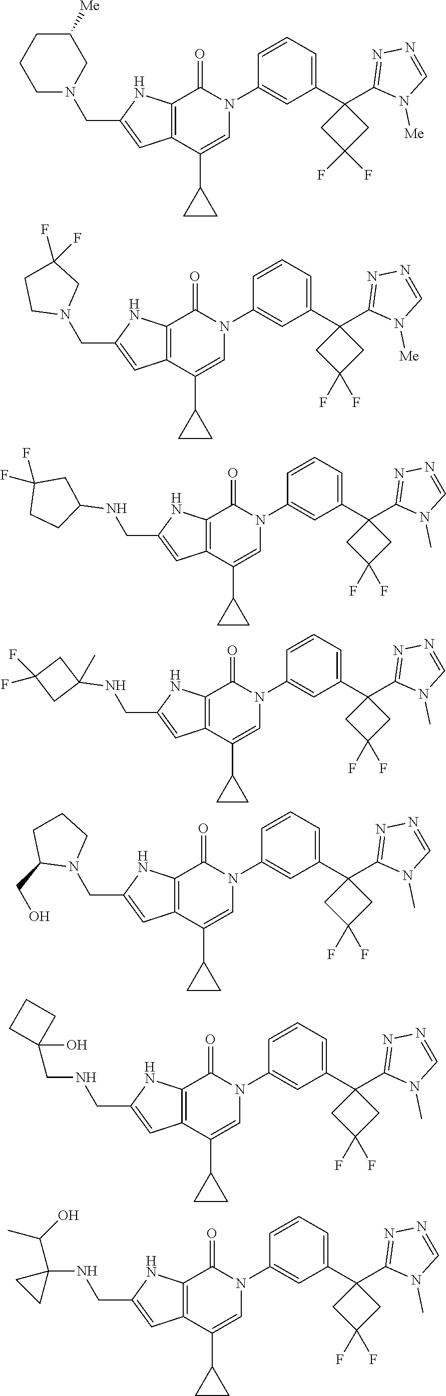 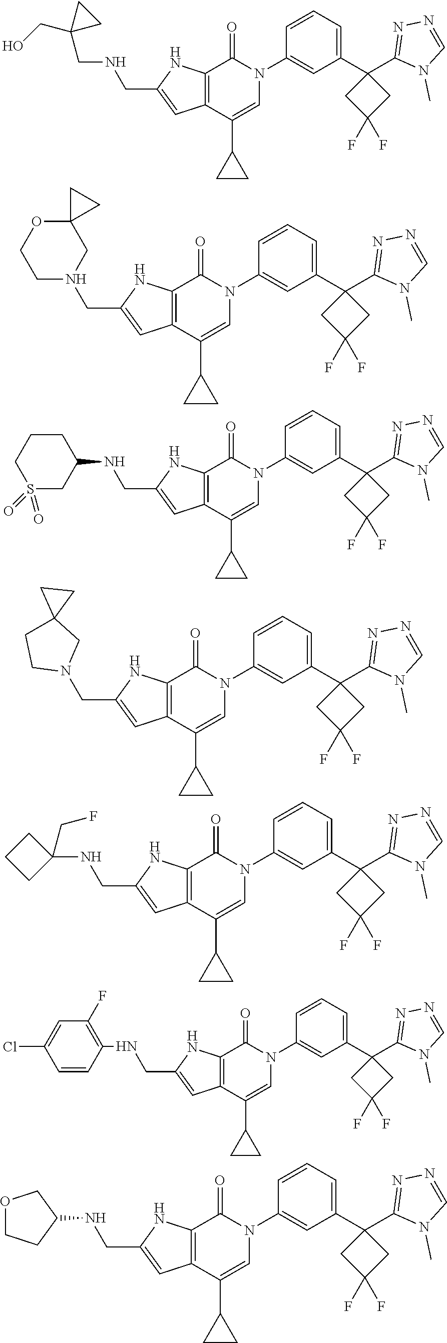 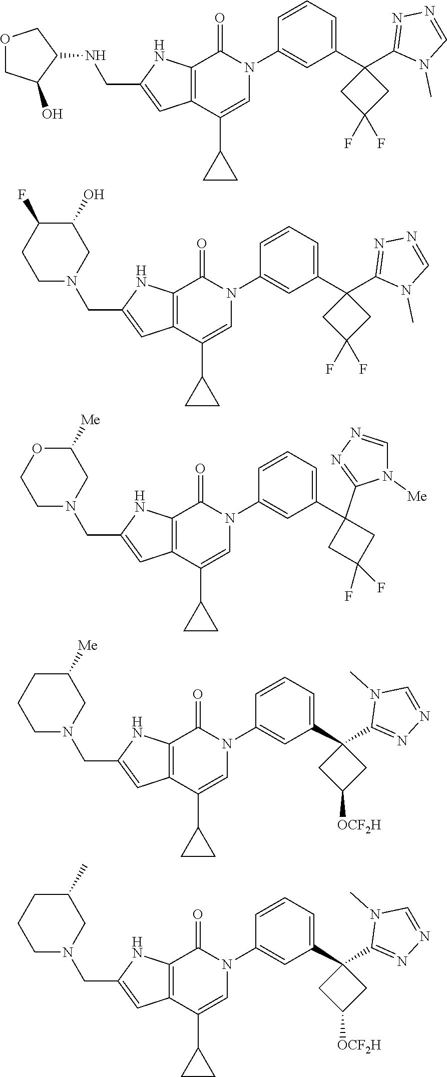 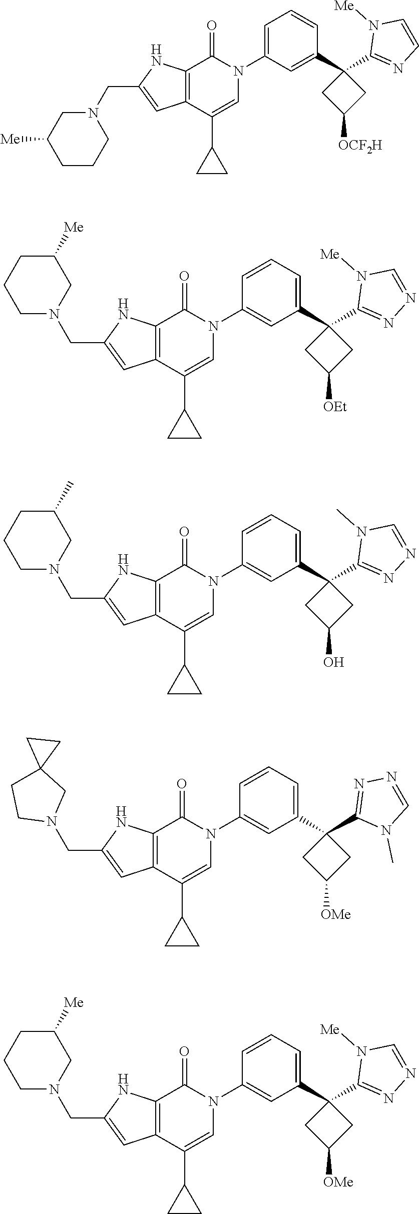 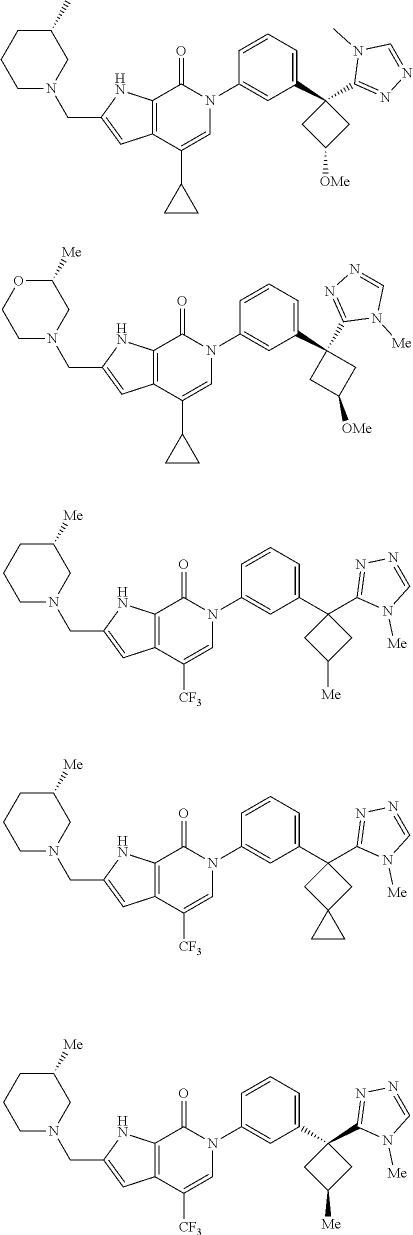 [SMILES: CO[C@H]1C[C@@](c2cccc(-n3cc(C4CC4)c4cc(CN5CCC[C@H](C)C5)[nH]c4c3=O)c2)(c2nncn2C)C1 ; CO[C@H]1C[C@](c2cccc(-n3cc(C4CC4)c4cc(CN5CCO[C@H](C)C5)[nH]c4c3=O)c2)(c2nncn2C)C1 ; CC1CC(c2cccc(-n3cc(C(F)(F)F)c4cc(CN5CCC[C@H](C)C5)[nH]c4c3=O)c2)(c2nncn2C)C1 ; C[C@H]1CCCN(Cc2cc3c(C(F)(F)F)cn(-c4cccc(C5(c6nncn6C)CC6(CC6)C5)c4)c(=O)c3[nH]2)C1 ; C[C@H]1CCCN(Cc2cc3c(C(F)(F)F)cn(-c4cccc([C@]5(c6nncn6C)C[C@@H](C)C5)c4)c(=O)c3[nH]2)C1] 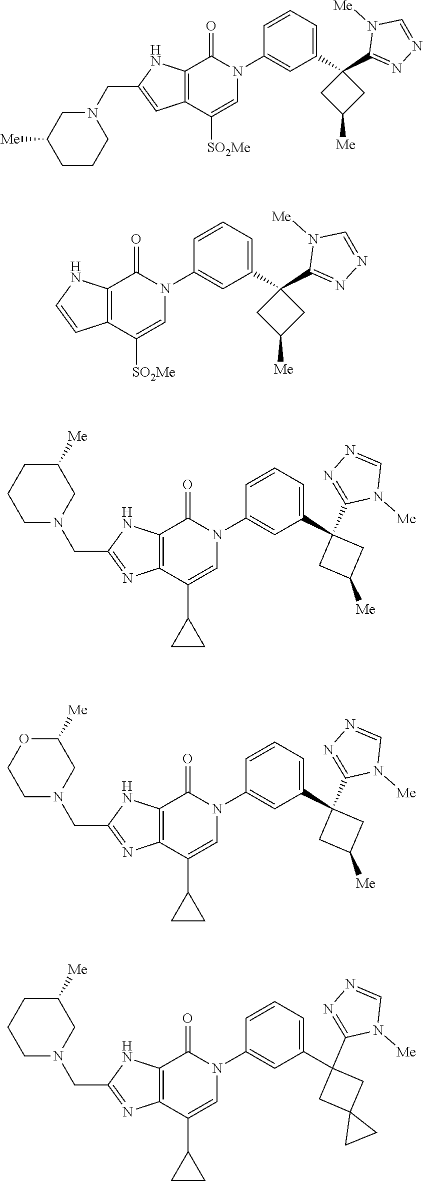 [SMILES: C[C@H]1CCCN(Cc2cc3c(S(C)(=O)=O)cn(-c4cccc([C@]5(c6nncn6C)C[C@@H](C)C5)c4)c(=O)c3[nH]2)C1 ; Cn1cnnc1[C@]1(c2cccc(-n3cc(S(C)(=O)=O)c4cc[nH]c4c3=O)c2)C[C@H](C)C1 ; C[C@H]1CCCN(Cc2nc3c(C4CC4)cn(-c4cccc([C@]5(c6nncn6C)C[C@H](C)C5)c4)c(=O)c3[nH]2)C1 ; C[C@@H]1CN(Cc2nc3c(C4CC4)cn(-c4cccc([C@]5(c6nncn6C)C[C@H](C)C5)c4)c(=O)c3[nH]2)CCO1 ; C[C@H]1CCCN(Cc2nc3c(C4CC4)cn(-c4cccc(C5(c6nncn6C)CC6(CC6)C5)c4)c(=O)c3[nH]2)C1] 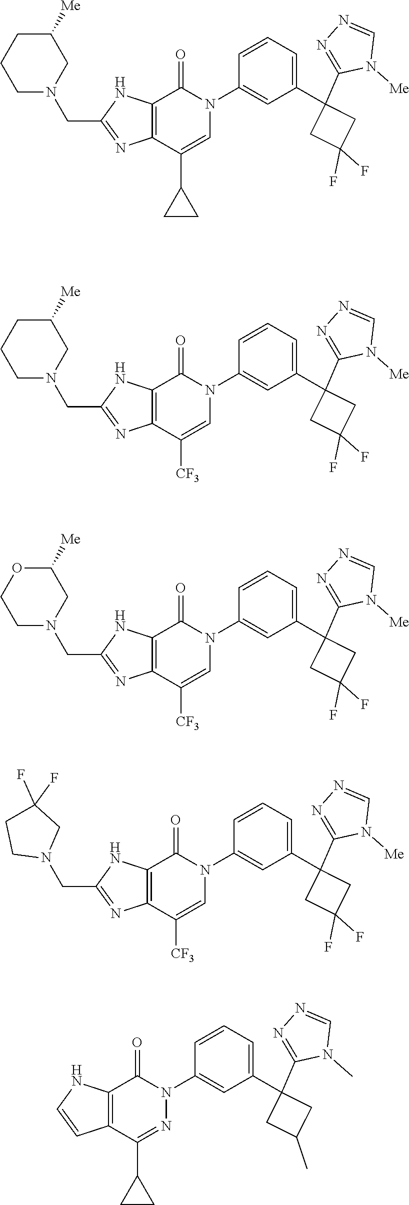 [SMILES: C[C@H]1CCCN(Cc2nc3c(C4CC4)cn(-c4cccc(C5(c6nncn6C)CC(F)(F)C5)c4)c(=O)c3[nH]2)C1 ; C[C@H]1CCCN(Cc2nc3c(C(F)(F)F)cn(-c4cccc(C5(c6nncn6C)CC(F)(F)C5)c4)c(=O)c3[nH]2)C1 ; C[C@@H]1CN(Cc2nc3c(C(F)(F)F)cn(-c4cccc(C5(c6nncn6C)CC(F)(F)C5)c4)c(=O)c3[nH]2)CCO1 ; Cn1cnnc1C1(c2cccc(-n3cc(C(F)(F)F)c4nc(CN5CCC(F)(F)C5)[nH]c4c3=O)c2)CC(F)(F)C1 ; CC1CC(c2cccc(-n3nc(C4CC4)c4cc[nH]c4c3=O)c2)(c2nncn2C)C1] 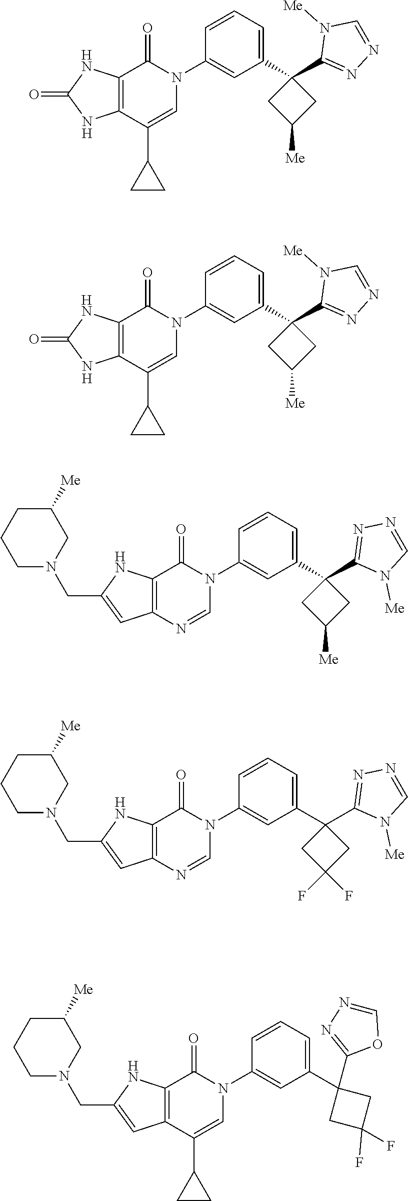 [SMILES: Cn1cnnc1[C@]1(c2cccc(-n3cc(C4CC4)c4[nH]c(=O)[nH]c4c3=O)c2)C[C@H](C)C1 ; Cn1cnnc1[C@]1(c2cccc(-n3cc(C4CC4)c4[nH]c(=O)[nH]c4c3=O)c2)C[C@@H](C)C1 ; C[C@H]1CCCN(Cc2cc3ncn(-c4cccc([C@]5(c6nncn6C)C[C@@H](C)C5)c4)c(=O)c3[nH]2)C1 ; C[C@H]1CCCN(Cc2cc3ncn(-c4cccc(C5(c6nncn6C)CC(F)(F)C5)c4)c(=O)c3[nH]2)C1 ; C[C@H]1CCCN(Cc2cc3c(C4CC4)cn(-c4cccc(C5(c6nnco6)CC(F)(F)C5)c4)c(=O)c3[nH]2)C1] 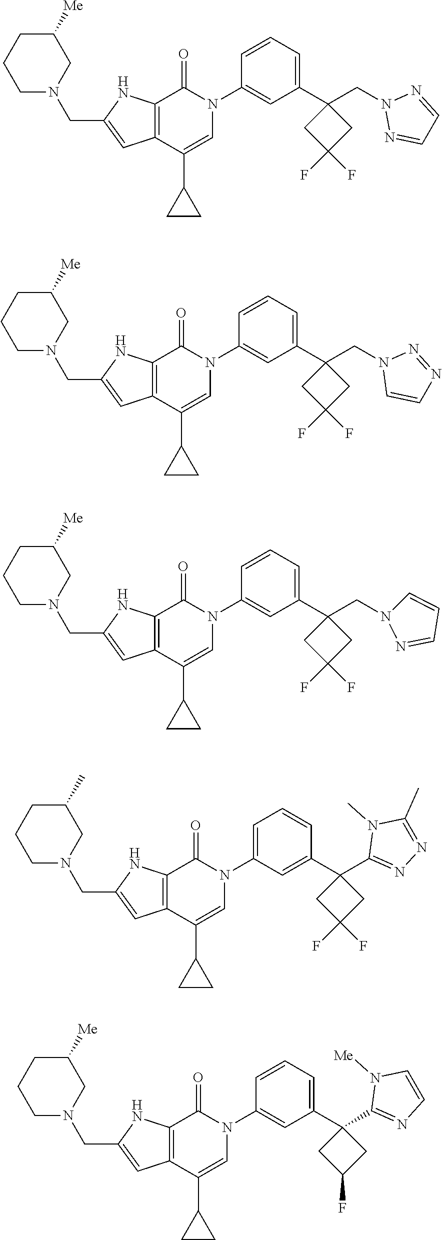 [SMILES: C[C@H]1CCCN(Cc2cc3c(C4CC4)cn(-c4cccc(C5(Cn6nccn6)CC(F)(F)C5)c4)c(=O)c3[nH]2)C1 ; C[C@H]1CCCN(Cc2cc3c(C4CC4)cn(-c4cccc(C5(Cn6ccnn6)CC(F)(F)C5)c4)c(=O)c3[nH]2)C1 ; C[C@H]1CCCN(Cc2cc3c(C4CC4)cn(-c4cccc(C5(Cn6cccn6)CC(F)(F)C5)c4)c(=O)c3[nH]2)C1 ; Cc1nnc(C2(c3cccc(-n4cc(C5CC5)c5cc(CN6CCC[C@H](C)C6)[nH]c5c4=O)c3)CC(F)(F)C2)n1C ; C[C@H]1CCCN(Cc2cc3c(C4CC4)cn(-c4cccc([C@]5(c6nccn6C)C[C@H](F)C5)c4)c(=O)c3[nH]2)C1] 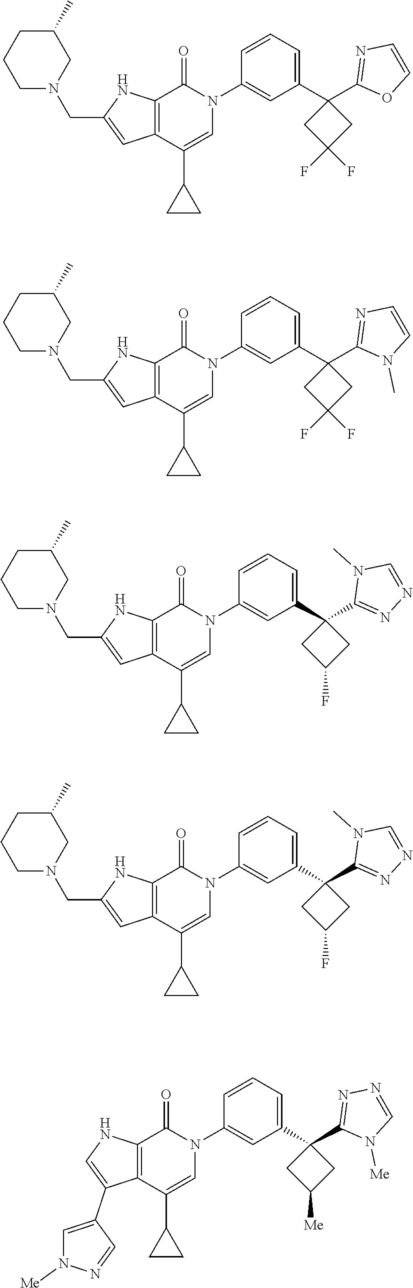 [SMILES: C[C@H]1CCCN(Cc2cc3c(C4CC4)cn(-c4cccc(C5(c6ncco6)CC(F)(F)C5)c4)c(=O)c3[nH]2)C1 ; C[C@H]1CCCN(Cc2cc3c(C4CC4)cn(-c4cccc(C5(c6nccn6C)CC(F)(F)C5)c4)c(=O)c3[nH]2)C1 ; C[C@H]1CCCN(Cc2cc3c(C4CC4)cn(-c4cccc([C@]5(c6nncn6C)C[C@@H](F)C5)c4)c(=O)c3[nH]2)C1 ; C[C@H]1CCCN(Cc2cc3c(C4CC4)cn(-c4cccc([C@]5(c6nncn6C)C[C@H](F)C5)c4)c(=O)c3[nH]2)C1 ; Cn1cc(-c2c[nH]c3c(=O)n(-c4cccc([C@]5(c6nncn6C)C[C@@H](C)C5)c4)cc(C4CC4)c23)cn1] 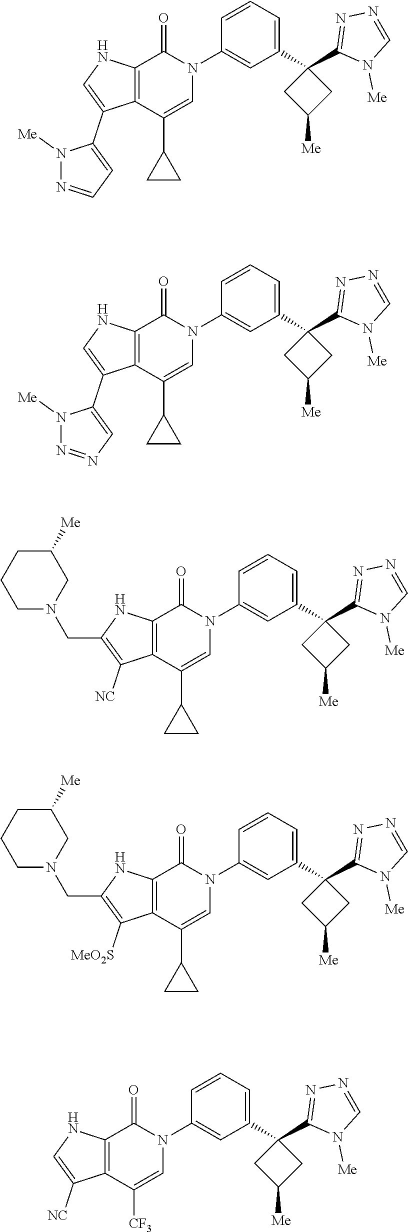 [SMILES: Cn1nccc1-c1c[nH]c2c(=O)n(-c3cccc([C@]4(c5nncn5C)C[C@@H](C)C4)c3)cc(C3CC3)c12 ; Cn1nncc1-c1c[nH]c2c(=O)n(-c3cccc([C@]4(c5nncn5C)C[C@@H](C)C4)c3)cc(C3CC3)c12 ; C[C@H]1CCCN(Cc2[nH]c3c(=O)n(-c4cccc([C@]5(c6nncn6C)C[C@@H](C)C5)c4)cc(C4CC4)c3c2C#N)C1 ; C[C@H]1CCCN(Cc2[nH]c3c(=O)n(-c4cccc([C@]5(c6nncn6C)C[C@@H](C)C5)c4)cc(C4CC4)c3c2S(C)(=O)=O)C1 ; Cn1cnnc1[C@]1(c2cccc(-n3cc(C(F)(F)F)c4c(C#N)c[nH]c4c3=O)c2)C[C@H](C)C1] 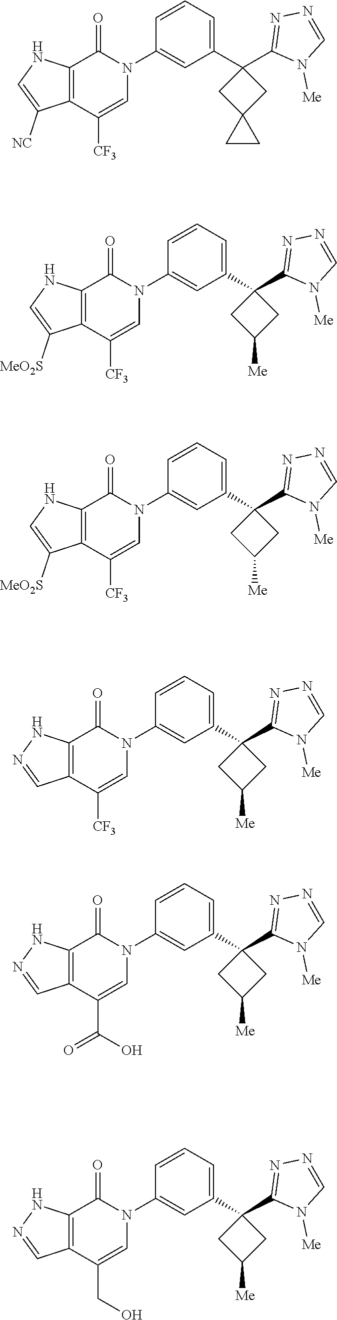 [SMILES: Cn1cnnc1C1(c2cccc(-n3cc(C(F)(F)F)c4c(C#N)c[nH]c4c3=O)c2)CC2(CC2)C1 ; Cn1cnnc1[C@]1(c2cccc(-n3cc(C(F)(F)F)c4c(S(C)(=O)=O)c[nH]c4c3=O)c2)C[C@H](C)C1 ; Cn1cnnc1[C@]1(c2cccc(-n3cc(C(F)(F)F)c4c(S(C)(=O)=O)c[nH]c4c3=O)c2)C[C@@H](C)C1 ; Cn1cnnc1[C@]1(c2cccc(-n3cc(C(F)(F)F)c4cn[nH]c4c3=O)c2)C[C@H](C)C1 ; Cn1cnnc1[C@]1(c2cccc(-n3cc(C(=O)O)c4cn[nH]c4c3=O)c2)C[C@H](C)C1 ; Cn1cnnc1[C@]1(c2cccc(-n3cc(CO)c4cn[nH]c4c3=O)c2)C[C@H](C)C1] 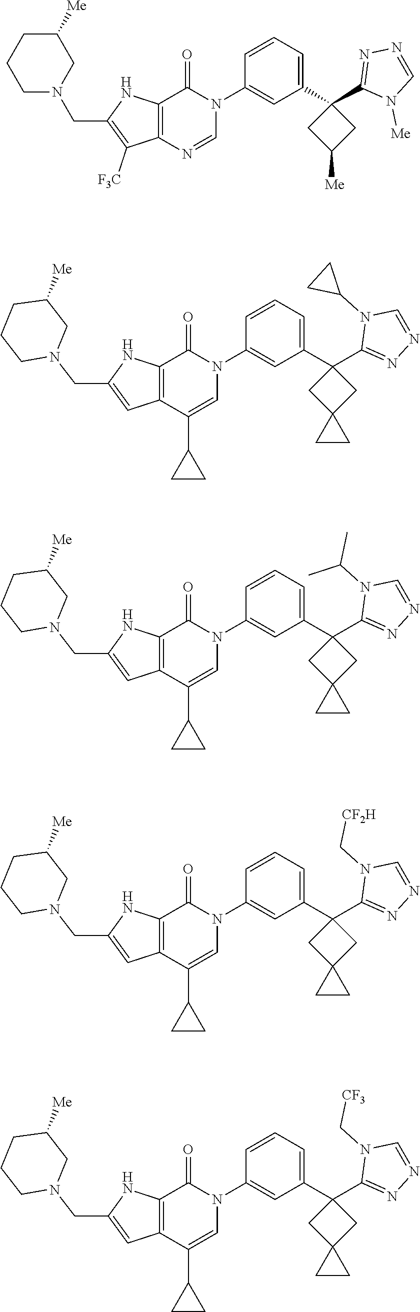 [SMILES: C[C@H]1CCCN(Cc2[nH]c3c(=O)n(-c4cccc([C@]5(c6nncn6C)C[C@@H](C)C5)c4)cnc3c2C(F)(F)F)C1 ; C[C@H]1CCCN(Cc2cc3c(C4CC4)cn(-c4cccc(C5(c6nncn6C6CC6)CC6(CC6)C5)c4)c(=O)c3[nH]2)C1 ; CC(C)n1cnnc1C1(c2cccc(-n3cc(C4CC4)c4cc(CN5CCC[C@H](C)C5)[nH]c4c3=O)c2)CC2(CC2)C1 ; C[C@H]1CCCN(Cc2cc3c(C4CC4)cn(-c4cccc(C5(c6nncn6CC(F)F)CC6(CC6)C5)c4)c(=O)c3[nH]2)C1 ; C[C@H]1CCCN(Cc2cc3c(C4CC4)cn(-c4cccc(C5(c6nncn6CC(F)(F)F)CC6(CC6)C5)c4)c(=O)c3[nH]2)C1] 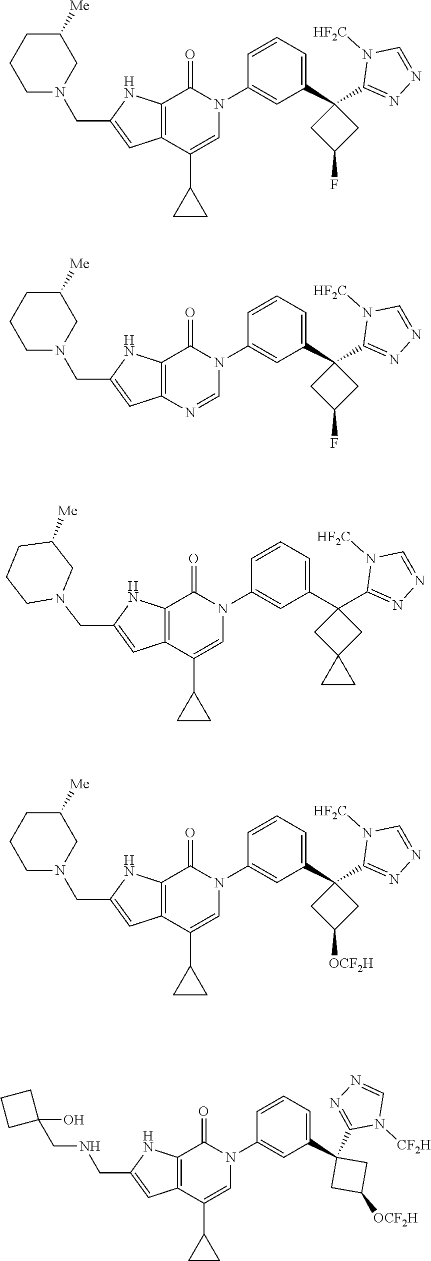 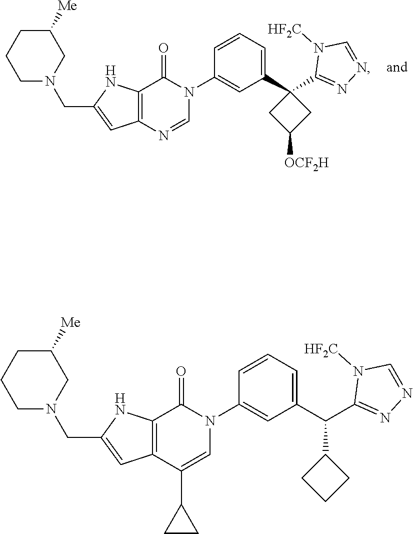
or a pharmaceutically acceptable salt thereof.

48. The compound of claim 1 selected from the group consisting of: 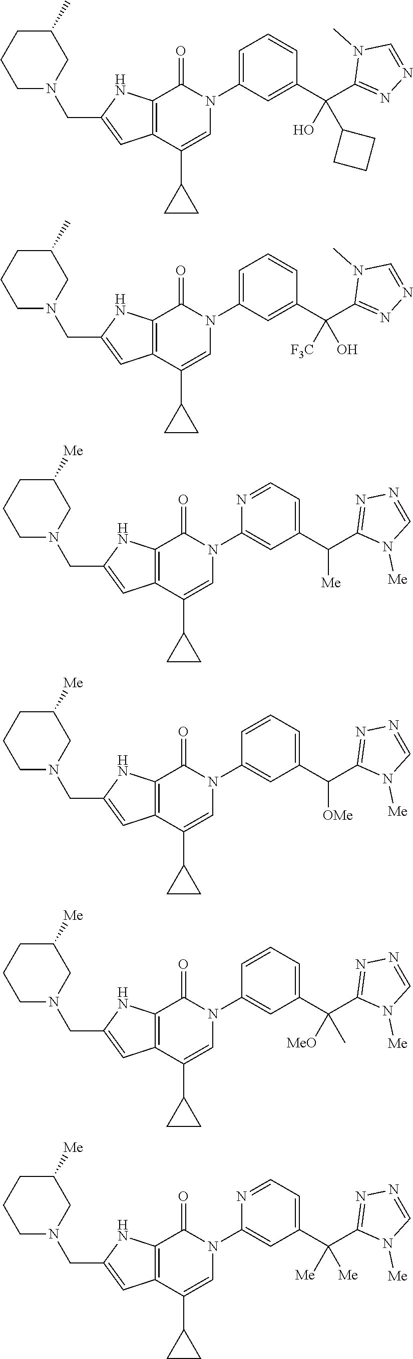 [SMILES: C[C@H]1CCCN(Cc2cc3c(C4CC4)cn(-c4cccc(C(O)(c5nncn5C)C5CCC5)c4)c(=O)c3[nH]2)C1 ; C[C@H]1CCCN(Cc2cc3c(C4CC4)cn(-c4cccc(C(O)(c5nncn5C)C(F)(F)F)c4)c(=O)c3[nH]2)C1 ; CC(c1ccnc(-n2cc(C3CC3)c3cc(CN4CCC[C@H](C)C4)[nH]c3c2=O)c1)c1nncn1C ; COC(c1cccc(-n2cc(C3CC3)c3cc(CN4CCC[C@H](C)C4)[nH]c3c2=O)c1)c1nncn1C ; COC(C)(c1cccc(-n2cc(C3CC3)c3cc(CN4CCC[C@H](C)C4)[nH]c3c2=O)c1)c1nncn1C ; C[C@H]1CCCN(Cc2cc3c(C4CC4)cn(-c4cc(C(C)(C)c5nncn5C)ccn4)c(=O)c3[nH]2)C1] 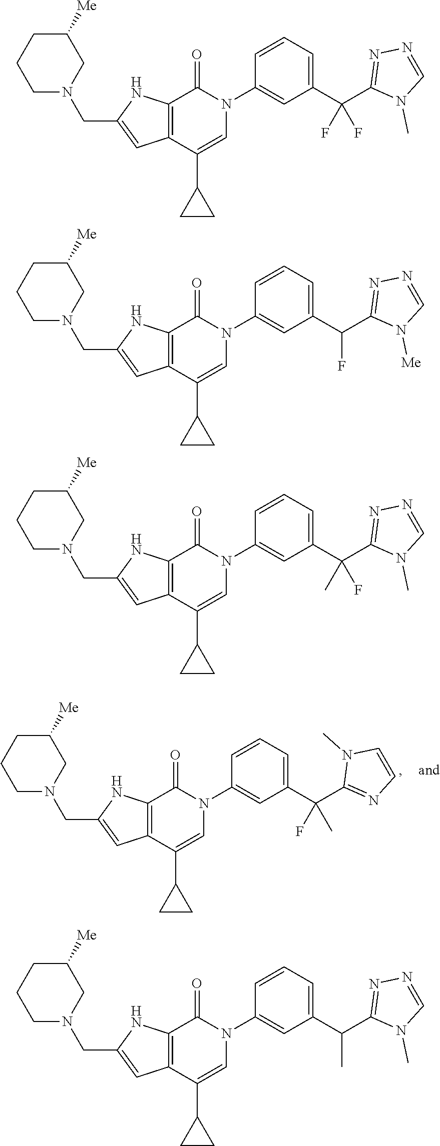
or a pharmaceutically acceptable salt thereof.

49. A pharmaceutical composition comprising a compound or pharmaceutically acceptable salt of claim 1, and a pharmaceutically acceptable excipient.

50-51. (canceled)

52. A method of treating a disease, disorder, or condition mediated at least in part by Cbl-b in a subject in need thereof, the method comprising administering to the subject a therapeutically effective amount of the compound of claim 1.

53. (canceled)

54. The method of claim 52, wherein the disease, disorder, or condition is cancer.

55-57. (canceled)

58. The method of claim 54, further comprising administering at least one additional therapeutic agent to the subject.

59-93. (canceled)
